(19) United States
(12) Patent Application Publication
(10) Pub. No.: US 2024/0238422 A1
(43) Pub. Date: Jul. 18, 2024

(54) Hypoimmunogenic Biotherapeutics

(71) Applicant: Osprey Biopharmaceuticals, Inc., San Diego, CA (US)

(72) Inventors: Richard James Glynne, La Jolla, CA (US); Tigran Aivazian, San Diego, CA (US)

(21) Appl. No.: 18/036,844

(22) PCT Filed: Jan. 10, 2022
(86) PCT No.: PCT/US2022/011869

§ 371 Date: May 12, 2023

Related U.S. Application Data
(60) Provisional application No. 63/136,128, filed on Jan. 11, 2021.

(51) Int. Cl. A61K 47/54; A61K 39/00; A61K 47/64; A61K 47/65; A61K 47/68; A61P 37/06; C07K 14/705; C07K 16/24; C07K 16/42

(52) U.S. Cl. CPC A61K 47/549; A61K 47/54; A61K 47/545; A61K 47/64; A61K 47/65; A61K 47/6845; A61P 37/06; C07K 14/70503; C07K 16/241; C07K 16/4283; A61K 2039/505

(57) ABSTRACT

The present disclosure provides hypoimmunogenic biotherapeutic compositions that suppress the development of an immune response to themselves in an individual. The present disclosure also provides pharmaceutical compositions that include such hypoimmunogenic biotherapeutics, methods for making such hypoimmunogenic biotherapeutics, and methods for using such hypoimmunogenic biotherapeutics as therapeutics and in research.

### CROSS-REFERENCE TO RELATED APPLICATIONS

This application claims the benefit of U.S. Provisional Application No. 63/136,128, filed Jan. 11, 2021, the disclosure of which is incorporated herein by reference in its entirety.

### FIELD OF THE INVENTION

This invention pertains to biotherapeutics that suppress an immune response to themselves in patients.

### BACKGROUND OF THE INVENTION

Therapeutic proteins and gene therapies are novel and successful drug modalities for the treatment of disease. However, patient immune responses to such therapeutics often result in inhibition of drug activity, accelerated drug clearance, compromised drug safety, and loss of drug efficacy. Prevention of the formation of neutralizing and non-neutralizing drug-specific antibodies (“anti-drug antibodies” or “ADA”) is a key unsolved problem in the field of biotherapeutics. Blocking ADA responses to biotherapeutics would improve drug exposure, improve durability of efficacy, reduce ADA-related toxicities, and enable favorable pharmacology for otherwise undruggable modalities (e.g., de novo designed drugs, drugs based on endogenous proteins). The present invention addresses these issues.

### SUMMARY OF THE INVENTION

The present disclosure provides hypoimmunogenic biotherapeutic compositions that suppress the development of an immune response to themselves in an individual. The present disclosure also provides pharmaceutical compositions that include such hypoimmunogenic biotherapeutics, methods for making such hypoimmunogenic biotherapeutics, and methods for using such hypoimmunogenic biotherapeutics as therapeutics and in research.

### BRIEF DESCRIPTION OF THE DRAWINGS

FIG. 1 depicts an aspect of the model for CD22-engaging biotherapeutics with suppressed anti-drug antibody responses: B cell receptor-Siglec Ligand co-engagers (including Drug-Siglec Ligand conjugates) suppress or silence drug-specific B cell activation by virtue of the physical recruitment of the inhibitory CD22 receptor to the B cell receptor complex.

FIG. 2 depicts another aspect of the model for CD22-engaging biotherapeutics with suppressed anti-drug antibody responses: B cell receptor-Siglec Ligand co-engagers (including Drug-Siglec Ligand conjugates) suppress, silence, or delete only drug-specific B cells while leaving intact those B cell clones not specific for drug.

FIG. 3 depicts multiple formats for Siglec-B cell receptor-co-engaging biologics with reduced immunogenicity: Format 1. Siglec Ligand-Modified Protein: Non-Enzymatic or enzymatic conjugation. Site-specific or non-site specific. Format 2. Siglec Ligand-Modified Glycan (in-cell or in vitro): Biosynthetic, enzymatic glycan modification (e.g., during protein expression). Format 3. Siglec Ligand-Modified Glycan (in vitro): Post expression, in vitro Enzymatic Glycan Modification. Format 4. Protein/Peptide Fusion: CD22-Binding Binding Domains or Peptides are incorporated into the biologic through in-frame fusion or conjugation.

FIG. 4 depicts an example conjugatable, CD22-binding, Siglec Ligand-linker structure, highlighting the components of the structure: Siglec Ligand binding moiety, Siglec Ligand-proximal linker structure, Linker, and reactive/conjugatable group.

FIG. 5 depicts example Siglec Ligand structures, focusing on elements that determine Siglec-2 binding affinity and species specificity.

FIG. 6 depicts example Siglec Ligand structures, showing structures varying in Siglec Ligand valency.

FIG. 7 depicts example Siglec Ligand structures, showing structures varying in linker structure, where a region proximal to the sialic acid-based moiety consists of either a PEG-based structure or a galactose-based structure.

FIG. 8 depicts example conjugatable linker structures potentiated (top) or not potentiated (middle) for Siglec-2 binding, and a negative control linker structure that does not bind Siglec-2 (bottom). Shown are a potentiated Siglec-ligand linker structure (Cpd. No. 26288, Siglec Ligand: Methyl-α-9-N-(biphenyl-4-carbonyl)-amino-9-deoxy-N-glycolylneuraminic acid) (top), a Siglec ligand-linker structure that contains a non-potentiated Siglec-2 binding moiety (Cpd. No. 26614, Siglec Ligand: N-glycolyl neuraminic acid/Neu5Gc) (middle), and a PEG-based non-Siglec binding conjugatable linker structure (Cpd. No. 26530) (bottom).

FIGS. 9A and 9B depict example purity and physicochemical characterization data for Adalimumab hIgG1-Siglec Ligand conjugates. Adalimumab conjugates vary in the structure of the Siglec-Linker used and the Ligand/Linker-to-Drug Ratio (“LDR”) after conjugation. FIG. 9A—capillary gel electrophoresis data for adalimumab conjugates. FIG. 9B—analytical size exclusion chromatography profiles for adalimumab IgG and adalimumab conjugates.

FIGS. 10A and 10B depict an in vitro B cell activation assay where mouse primary B cells are treated with either a B cell receptor agonizing anti-IgD antibody or anti-IgD-Siglec Ligand conjugates. The B cell stimulatory activities of anti-IgD and anti-IgD-Siglec Ligand conjugates are compared in dose titration experiments with an activation readout of CD69 upregulation. CD69 levels on the different treatment groups are evaluated through the percentage of cells that are CD69-positive (FIG. 10A) or the CD69 mean fluorescence intensity (MFI) (FIG. 10B). Siglec ligands in the tested conjugates vary in linker structure (“PEG” or “Gal”) and valency (“Monovalent” or “Bivalent”), and conjugates vary also in the Ligand/Linker-to-Drug Ratio (“LDR”), or the average number of Siglec Ligand structures per drug molecule.

FIGS. 11A and 11B depict an in vitro B cell activation assay where mouse primary B cells are treated with either a B cell receptor agonizing anti-IgD antibody or anti-IgD-Siglec Ligand conjugates. Anti-IgD antibody-Siglec Ligand conjugates bear trivalent Siglec Ligand Structures. B cell activation is measured through the upregulation of CD69 expression, as measured by cytometry. CD69 levels on the different treatment groups are evaluated through the percentage of cells that are CD69-positive (FIG. 11A) or the CD69 mean fluorescence intensity (MFI) (FIG. 11B). Siglec ligands in the tested conjugates vary in linker structure (“PEG” or “Gal”) and conjugates vary also in the Ligand/Linker-to-Drug Ratio (“LDR”), or the average number of Siglec Ligand structures per drug molecule.

FIGS. 12A and 12B depict an in vitro B cell activation assay where mouse primary B cells are treated with either a B cell receptor agonizing anti-IgD antibody or anti-IgD-Siglec Ligand conjugates. The B cell stimulatory activities of anti-IgD and anti-IgD-Siglec Ligand conjugates are compared in dose titration experiments with an activation readout of CD69 upregulation. CD69 levels on the different treatment groups are evaluated through the percentage of cells that are CD69-positive (FIG. 12A) or the CD69 mean fluorescence intensity (MFI) (FIG. 12B). Siglec ligands in the tested conjugates contain PEG-based linker structures (“PEG”) and vary in valency (“Monovalent”, “Bivalent”, and “Trivalent”). Conjugates vary also in the Ligand/Linker-to-Drug Ratio (“LDR”), or the average number of Siglec Ligand structures per drug molecule.

FIGS. 13A and 13B depict the evaluation of Siglec Ligand-anti-IgD antibody conjugate binding to mouse primary B cells, in comparison with the parental, unconjugated anti-IgD antibody. Binding is evaluated by fluorescence cytometry, in a competition assay format with Alexa-647-labeled anti-IgD antibody. FIG. 13A depicts dose-response results for concentration-dependent inhibition of fluorescently-labeled anti-IgD binding to IgD⁺ B cells by unlabeled anti-IgD-Siglec Ligand conjugates and unconjugated, unlabeled anti-IgD antibody. The binding IC50, in nanomolar, for each unlabeled test article is indicated. FIG. 13B is a schematic for the binding assay system.

FIG. 14 depict an in vitro B cell activation assay where mouse primary B cells are treated with either a B cell receptor agonizing anti-IgD antibody or anti-IgD-Siglec Ligand conjugates, where the same test articles are used to treat B cells from wild-type mice. The conjugate Siglec Ligands (“Monovalent PEG-LDR 9”, “Bivalent PEG-LDR 6”, “Trivalent PEG-LDR 6”, and “Trivalent PEG-LDR 8”) are potentiated for Siglec-2 binding. The B cell stimulatory activities of anti-IgD and anti-IgD-Siglec Ligand conjugates are compared in dose titration experiments with an activation readout of CD69 upregulation. CD69 levels on the different treatment groups are evaluated through the percentage of cells that are CD69-positive. Siglec ligands in the tested conjugates contain PEG-based linker structures (“PEG”) and vary in valency (“Monovalent”, “Bivalent”, and “Trivalent”). Conjugates vary also in the Ligand/Linker-to-Drug Ratio (“LDR”), or the average number of Siglec Ligand structures per drug molecule.

FIG. 15 depicts the evaluation of Siglec Ligand-anti-IgD antibody conjugate binding to mouse primary B cells, in comparison with the parental, unconjugated anti-IgD antibody. Binding is evaluated by fluorescence cytometry, in a competition assay format with Alexa-647-labeled anti-IgD antibody. Dose-response results are shown for concentration-dependent inhibition of fluorescently-labeled anti-IgD binding to IgD⁺ B cells by unlabeled anti-IgD-Siglec Ligand conjugates and unconjugated, unlabeled anti-IgD antibody. The binding IC50, in nanomolar, for each unlabeled test article is indicated. The test articles are identical to those in FIG. 15.

FIGS. 16A and 16B depict an in vitro B cell activation assay where mouse primary B cells are treated with either a B cell receptor agonizing anti-IgD antibody or anti-IgD-Siglec Ligand conjugates, where the Siglec Ligands are potentiated (“BPC-Neu5Gc Monovalent PEG-LDR 9” and “BPC-Neu5Gc Bivalent PEG-LDR 6”) or unpotentiated (“Neu5Gc Monovalent PEG-LDR 10” and “Neu5Gc Bivalent PEG-LDR 7”) for Siglec-2 binding. The B cell stimulatory activities of anti-IgD and anti-IgD-Siglec Ligand conjugates are compared in dose titration experiments with an activation readout of CD69 upregulation. CD69 levels on the different treatment groups are evaluated through the percentage of cells that are CD69-positive. Dose response curves are shown for monovalent (FIG. 16A) and bivalent (FIG. 16B) Siglec Ligand conjugates. Conjugates vary also in the Ligand/Linker-to-Drug Ratio (“LDR”), or the average number of Siglec Ligand structures per drug molecule.

FIGS. 17A to 17C depict an in vitro primary mouse B cell activation assay testing for the importance of CD22 engagement for Siglec Ligand-conjugate-mediated suppression of B cell receptor activation. Mouse primary B cells were treated with either a B cell receptor agonizing anti-IgD antibody or various anti-IgD-Siglec Ligand conjugates, bearing either potentiated Siglec linkers (“BPC-Neu5Gc Monovalent PEG-LDR 9”, “BPC-Neu5Gc Bivalent PEG-LDR 6”, “BPC-Neu5Gc Trivalent PEG-LDR 6”, and “BPC-Neu5Gc Trivalent PEG-LDR 8”), or asialo linkers lacking Siglec binding determinants (“Asialo Monovalent PEG-LDR 7”, “Asialo Bivalent PEG-LDR 8”, and “Asialo Trivalent PEG-LDR 7”). The B cell stimulatory activities of anti-IgD and anti-IgD-Siglec Ligand conjugates are compared in dose titration experiments with an activation readout of CD69 upregulation. CD69 levels on the different treatment groups are evaluated through the percentage of cells that are CD69-positive. Siglec ligands in the tested conjugates contain PEG-based linker structures (“PEG”) and vary in valency (“Monovalent” (FIG. 17A), “Bivalent” (FIG. 17B), and “Trivalent” (FIG. 17C)). Conjugates vary also in the Ligand/Linker-to-Drug Ratio (“LDR”), or the average number of Siglec Ligand structures per drug molecule.

FIGS. 18A to 18C depict the evaluation of Siglec Ligand-anti-IgD antibody conjugate binding to B cells relative to parental anti-IgD antibody. Anti-IgD-Siglec Ligand conjugates bear either potentiated Siglec Linkers (“BPC-Neu5Gc Monovalent PEG-LDR 9”, “BPC-Neu5Gc Bivalent PEG-LDR 6”, “BPC-Neu5Gc Trivalent PEG-LDR 6”, and “BPC-Neu5Gc Trivalent PEG-LDR 8”) or asialo linkers lacking Siglec binding determinants (“Asialo Monovalent PEG-LDR 7”, “Asialo Bivalent PEG-LDR 8”, and “Asialo Trivalent PEG-LDR 7”). Binding is evaluated by fluorescence cytometry, in a competition assay format with Alexa-647-labeled anti-IgD antibody. Dose-response results are shown for concentration-dependent inhibition of fluorescently-labeled anti-IgD binding to IgD⁺ B cells by unlabeled anti-IgD-Siglec Ligand conjugates and unconjugated, unlabeled anti-IgD antibody. The test articles are identical to those used for FIG. 17. Siglec ligands in the tested conjugates contain PEG-based linker structures (“PEG”) and vary in valency (“Monovalent” (FIG. 18A), “Bivalent” (FIG. 18B), and “Trivalent” (FIG. 18C)). Conjugates vary also in the Ligand/Linker-to-Drug Ratio (“LDR”), or the average number of Siglec Ligand structures per drug molecule.

FIGS. 19A and 19B depict an in vitro primary mouse B cell activation assay testing for the importance of cis B cell receptor and CD22 co-engagement for suppression of B cell receptor activation. FIG. 19A depicts a model for B cell receptor activation where the anti-IgD BCR agonist and Siglec-2-engaging moieties are presented on the same or separate molecules. The B cell stimulatory activities of anti-IgD, anti-IgD-Siglec Ligand conjugate, or a mixture of 2 nM anti-IgD and varying concentrations of control antibody-Siglec Ligand conjugate are compared in dose titration experiments with an activation readout of CD69 upregulation. CD69 levels on the different treatment groups are evaluated through the CD69 mean fluorescence intensity (MFI) (FIG. 19B). Siglec ligands in the tested conjugates contain PEG-based linker structures (“PEG”) and bear bivalent Siglec Ligand structures.

FIG. 20 depicts an in vitro primary mouse B cell activation assay testing for BCR agonism suppression in mixtures of agonistic anti-IgD antibody and non-agonistic Siglec Ligand-anti-IgD conjugate. Siglec Ligand-anti-IgD conjugate is titrated in the presence or absence of 2 nM anti-IgD BCR agonist. The B cell stimulatory activities of anti-IgD, anti-IgD-Siglec Ligand conjugate, or a mixture of 2 nM anti-IgD with varying concentrations of anti-IgD-Siglec Ligand conjugate are compared in dose titration experiments with an activation readout of CD69 upregulation. CD69 levels on the different treatment groups are evaluated through the CD69 mean fluorescence intensity (MFI). The anti-IgD conjugate tested bears a bivalent, Siglec-2-potentiated ligand (MPB-Neu5Ac), and a PEG-based linker, with a Ligand/Linker-to-Drug Ratio (“LDR”) of 6.

FIGS. 21A and 21B depict an in vitro B cell activation assay, using human primary B cells and either a B cell receptor agonizing anti-IgM antibody or Siglec Ligand conjugates with the same anti-IgM antibody. The B cell stimulatory activities of anti-IgM and anti-IgM-Siglec Ligand conjugates are compared in dose titration experiments with an activation readout of CD69 upregulation. CD69 levels on the different treatment groups are evaluated through the percentage of cells that are CD69-positive. Results with conjugates using galactose-based linkers (FIG. 21A) and PEG-based linkers (FIG. 21B) are shown. All conjugates bear Siglec-2-potentiated MPB-Neu5Ac structures, with varying valency and Ligand/Linker-to-Drug Ratio (“LDR”), or the average number of Siglec Ligand structures per drug molecule.

FIGS. 22A and 22B depict an in vitro B cell activation assay, using human primary B cells and either a B cell receptor agonizing anti-IgM antibody or Siglec Ligand conjugates with the same anti-IgM antibody. Conjugates bear Siglec Ligand structures that are either potentiated (“BPC-Neu5Gc Monovalent PEG-LDR5-αIgM” and “BPC-Neu5Gc Bivalent PEG-LDR9-αIgM”) or unpotentiated (“Neu5Gc Monovalent PEG-LDR6-αIgM” and “Neu5Gc Bivalent PEG-LDR6-αIgM”) for CD22 binding. The B cell stimulatory activities of anti-IgM and anti-IgM-Siglec Ligand conjugates are compared in dose titration experiments with an activation readout of CD69 upregulation. CD69 levels on the different treatment groups are evaluated through the percentage of cells that are CD69-positive. Siglec ligands in the tested conjugates contain PEG-based linker structures (“PEG”) and vary in valency (“Monovalent” (FIG. 22A) or “Bivalent” (FIG. 22B)).

FIGS. 23A and 23B depict evaluation of Siglec Ligand-anti-IgM antibody conjugate and parental anti-IgM binding to human B cells. Anti-IgM-Siglec Ligand are either potentiated (“BPC-Neu5Gc Monovalent PEG-LDR5-αIgM” and “BPC-Neu5Gc Bivalent PEG-LDR9-αIgM”) or unpotentiated (“Neu5Gc Monovalent PEG-LDR6-αIgM” and “Neu5Gc Bivalent PEG-LDR6-αIgM”) for CD22 binding. Binding is evaluated by fluorescence cytometry in a competition assay format with Alexa-647-labeled anti-IgM antibody. Dose-response results are shown for concentration-dependent inhibition of fluorescently-labeled anti-IgM binding to IgM⁻ B cells by unlabeled anti-IgM-Siglec Ligand conjugates and unconjugated, unlabeled anti-IgM antibody. The binding IC50, in nanomolar, for each unlabeled test article is indicated. The test articles are identical to those used in the experiment for FIG. 22.

FIGS. 24A to 24C depict evaluation of anti-drug antibody responses in mice for adalimumab and Siglec Ligand-adalimumab conjugates. Conjugates bear BPC-Neu5Gc-based Siglec Ligand-Linker structures that are either monovalent, bivalent, or trivalent for Siglec Ligands, with a galactose-containing linker structure. Mice received a single 4 mg/kg i.v. dose of adalimumab or adalimumab-Siglec Ligand conjugate. Serum IgG levels against adalimumab/adalimumab conjugates were measured at days 14, 21, and 28. FIG. 24A, FIG. 24B, and FIG. 241C show individual animal serum IgG titer values (n=5 mice) at days 14, 21, and 28, respectively.

FIGS. 25A to 25C depict evaluation of anti-drug antibody responses in mice for adalimumab and Siglec Ligand-adalimumab conjugates. Conjugates bear BPC-Neu5Gc-based Siglec Ligand-Linker structures that are either monovalent, bivalent, or trivalent for Siglec Ligands, with a PEG-containing linker structure. Mice received a single 4 mg/kg i.v. dose of adalimumab or adalimumab-Siglec Ligand conjugate. Serum IgG levels against adalimumab/adalimumab conjugates were measured at days 14, 21, and 28. FIG. 25A, FIG. 25B, and FIG. 25C show individual animal serum IgG titer values (n=5 mice) at days 14, 21, and 28, respectively.

FIGS. 26A and 26B depict analysis of serum pharmacokinetics for adalimumab hIgG and Siglec Ligand-adalimumab hIgG1 conjugates in mice. Conjugates bear BPC-Neu5Gc-based Siglec Ligand-Linker structures that are either monovalent, bivalent, or trivalent for Siglec Ligands, with a PEG-containing linker structure. Test articles are identical to those used in FIG. 25. After intravenous dosing, serum samples were analyzed for test article concentrations by anti-human IgG Fc ELISA (FIG. 26A) and SPR (surface plasmon resonance, FIG. 26B).

FIGS. 27A to 27G depict in vitro surface plasmon resonance (SPR) analysis of TNFα binding activity for adalimumab hIgG and Siglec Ligand-adalimumab hIgG1 conjugates. FIG. 27A is a schematic for the SPR assay setup, with TNFα analyte binding to Protein A chip-immobilized adalimumab or adalimumab-Siglec Ligand conjugate. TNFα concentration is varied to evaluate concentration dependence, binding kinetics, and affinity. FIG. 27B shows the concentration dependencies of binding at the end of the sensorgram association phase (RUₘₐₓ). FIG. 27C-G are individual sensorgrams for Adalimumab (FIG. 27C), Adalimumab BPC-Neu5Gc Monovalent GAL LDR 4 (FIG. 27D), Adalimumab BPC-Neu5Gc Monovalent GAL LDR 7 (FIG. 27E), Adalimumab BPC-Neu5Gc Bivalent GAL LDR 8.5 (FIG. 27F), and Adalimumab BPC-Neu5Gc Trivalent GAL LDR 5 (FIG. 27G).

FIGS. 28A and 28B depict evaluation of anti-drug antibody responses in mice for 1) adalimumab, 2) a Siglec Ligand-adalimumab conjugate (“BPC-Neu5Gc Monovalent PEG LDR 10”), and 3) negative control, non-Siglec-2-binding linker conjugates (“Neg Ctrl Monovalent PEG LDR 7”, “Neg Ctrl Bivalent PEG LDR 7”, and “Neg Ctrl Trivalent PEG LDR 6”. Mice received a single 4 mg/kg i.v. dose of adalimumab, adalimumab-Siglec Ligand conjugate, or adalimumab-negative control linker conjugate. Serum IgG levels against adalimumab/adalimumab conjugates were measured at days 14 and 28. FIG. 28A and FIG. 28B show individual animal serum IgG titer values (n=5 mice) at days 14 and 28, respectively.

FIG. 29 depicts evaluation of anti-drug antibody responses in mice for 1) adalimumab, 2) a potentiated Siglec Ligand-adalimumab conjugate (“BPC-Neu5Gc Monovalent PEG LDR 7”), and 3) Non-potentiated Siglec Ligand-adalimumab conjugates (“Neu5Gc Monovalent PEG LDR 6”, “Neu5Gc Bivalent PEG LDR 5”, and “Neu5Gc Trivalent PEG LDR 5”). Mice received a single 4 mg/kg i.v. dose of adalimumab IgG or conjugate. Serum IgG levels against adalimumab/adalimumab conjugates were measured at day 28. Individual animal serum IgG titer values (n=5 mice) are shown for day 28.

FIGS. 30A to 30E depict evaluation of IgG immune responses in mice dosed with different combinations of 4 mg/kg adalimumab, adalimumab-Siglec Ligand conjugate (Adalimumab-BPC-Neu5Gc Bivalent PEG LDR 10), or PBS vehicle (day 0), and subsequent weekly dosing with 200 μg hen egg white lysozyme (HEL) (days 7, 14, 21, and 28). The study plan is shown in FIG. 30A. Mice serum IgG titers were measured separately against adalimumab/adalimumab-Siglec Ligand conjugate and HEL. Day 28 serum IgG levels are shown for anti-adalimumab/adalimumab conjugate (FIG. 30B) and HEL (FIG. 30D). Titers are shown for Anti-adalimumab/adalimumab conjugates (FIG. 30C) and HEL (FIG. 30E).

FIGS. 31A to 31C depict the evaluation of anti-drug antibody responses in mice for HEL and a monovalent Siglec Ligand-HEL conjugate (“HEL-BPC-Neu5Gc Monovalent PEG LDR 1.6). The study plan is shown in FIG. 31A. Mice received 4 weekly 200 μg i.v. doses of HEL or HEL conjugate (days 0, 7, 14, and 21). Serum IgG levels against HEL/HEL conjugate were measured at day 27. FIG. 31B shows the IgG level dilution series from serum samples. FIG. 31C shows the individual animal day 27 serum IgG titer values (n=5 mice).

FIG. 32 depicts the evaluation of anti-drug antibody responses in mice for recombinant asparaginase enzyme and asparaginase-Siglec Ligand conjugates (“Asn'ase BPC-Neu5Gc Monovalent PEG-LDR 10” and “Asn'ase BPC-Neu5Gc Trivalent PEG-LDR 3.5”). BALB/c (n=5 per group) were dosed weekly (4 times) with 15 μg Asparaginase or Asparaginase conjugate. Anti-Asparaginase IgG titers were measured at day 28 by ELISA assay. Titers are shown for each test article.

### DETAILED DESCRIPTION OF THE INVENTION

Hypoimmunogenic biotherapeutic compositions are provided that suppress the development of an immune response to themselves in an individual. Also provided are pharmaceutical compositions comprising such hypoimmunogenic biotherapeutics, methods for making such hypoimmunogenic biotherapeutics, and methods for using such hypoimmunogenic biotherapeutics as therapeutics and in research. Such hypoimmunogenic biotherapeutics find particular use in the treatment of diseases that require repeat or chronic administration of the biotherapeutics therapeutic to be effective. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The transitional term “comprising” is inclusive or open-ended and does not exclude additional, unrecited elements or method steps. The transitional phrase “consisting of” excludes any element, step, or ingredient that is not specified. The transitional phrase “consisting essentially of” defines the scope to the specified elements, materials or steps and those that do not materially affect the basic and novel characteristics of the invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Compositions

Disclosed herein are engineered biotherapeutics that are hypoimmunogenic, methods for making such biotherapeutics, and methods for their use. As used herein, a “biotherapeutic” refers to a composition that is composed of sugars, amino acids, proteins, lipids or nucleic acids or complex combinations of these substances and that is therapeutic in an individual. Nonlimiting examples of biotherapeutics include protein therapeutics, e.g. antibody therapeutics, fusion protein therapeutics, enzyme therapeutics; viral therapeutics; cell therapeutics; and nucleic acid therapeutics. By an “engineered” biotherapeutic, it is meant a biotherapeutic that has been designed and built to comprise one or more modifications relative to biotherapeutic that has not been so engineered, i.e. a parental biotherapeutic, which is also referred to herein as an unengineered biotherapeutic. By a “hypoimmunogenic” composition, it is meant a composition that suppresses an unwanted, drug-specific immune response in an individual relative to a reference composition, e.g. a corresponding nonengineered composition, when administered to the individual; for example, reducing an immune response by 50% or more relative to a reference, e.g. a nonengineered biotherapeutic, in some instances 60%, 70%, 80% or more, for example 85%, 90%, 95% or more, in certain cases 98%, 99%, or 100%, i.e. such that the immune response is undetectable, i.e. the biotherapeutic is nonimmunogenic. Thus, disclosed herein are engineered biotherapeutics which retain pharmacologic activity while comprising one or more modifications that render the biotherapeutic capable of suppressing a drug-specific immune response in an individual to which it has been administered as compared to the unmodified biotherapeutic. In some embodiments, the immune response is a humoral immune response i.e., a B cell-driven response, e.g. an IgG response.

Disclosed herein is an engineered hypoimmunogenic biotherapeutic, comprising a biotherapeutic which has been engineered to comprise a modified Sialic acid-binding immunoglobulin-type lectin (Siglec) ligand profile relative to a corresponding unengineered biotherapeutic while retaining therapeutic activity.

By a “Siglec” it is meant a member of the family of proteins that are found primarily on the surface of leukocytes and that bind sialic acids on target biologics. By a “Siglec ligand profile”, it is meant the amount and/or location of Siglec ligands that are covalently bound to a biotherapeutic. In many instances, the modification is an increase in the amount and/or location of Siglec ligands that are covalently bound to a biotherapeutic, wherein the increase renders the biotherapeutic less immunogenic in an individual relative to the corresponding unmodified biotherapeutic.

There are 14 different mammalian Siglecs, which are expressed on different types of leukocytes and which may exert inhibitory or activating effects on the cells on which they are expressed depending on whether they comprise an inhibitory motif or activating motif. Siglecs show distinct binding preferences for different sialic acids, and the type of linkage and type of underlying sugar also affect recognition of sialic acids. (Varki, A. and Crocker, P. R. (2009) I-type lectins. In Essentials of Glycobiology (2nd edn) (Varki, A. et al., eds), pp. 459-474, Cold Spring Harbor Laboratory Press; Crocker, P. R. et al. (2007) Nat. Rev. Immunol. 7, 255-266). Together, this provides for an array of alternative Siglec ligands that may be deployed to modulate an immune response to a biotherapeutic. Of particular interest is the suppression of an immune response to a biotherapeutic, and more particularly, of a B cell response to the biotherapeutic. Accordingly, in some embodiments, the Siglec ligand is a ligand for a Siglec that is expressed on B lymphocytes, for example Siglec-2 (also called CD22), Siglec-5 (CD170), Siglec-6, Siglec-9 (CD329), or Siglec-10 (Siglec-G). In some embodiments, the Siglec is Siglec-2. In some embodiments, the Siglec is Siglec-5. In some embodiments, the Siglec is Siglec-6. In some embodiments, the Siglec is Siglec-9. In some embodiments, the Siglec is Siglec-10. In some embodiments, the hypoimmunogenic biotherapeutic has been engineered to comprise the sialic acid ligands for one Siglec. In other embodiments, the hypoimmunogenic biotherapeutic has been engineered to comprise the Siglec ligands for two or more Siglecs, e.g. for 3 Siglecs or for 4 Siglecs, in certain cases, for 5 Siglecs. In some cases there are 5, 6, 7, 8, 9, 10, 11, 12, 13, or 15 Siglecs.

In some cases, the engineered hypoimmunogenic biotherapeutic is of formula (I):

[Xₙ-L]ₘ-Y (I)

wherein X is a sialic acid group, L is an optional linker, Y is the biotherapeutic, and n is an integer of 1 or more, and m is an integer of 1 or more. The combination of X and L groups, i.e. [Xₙ-L], is collectively referred to as the Siglec ligand herein.

### Sialic Acid Group X

X is a sialic acid group, wherein the term “sialic acid” refers to alpha-keto acid sugars with a nine-carbon backbone. Thus, since X is a sialic acid group, X comprises a sialic acid or a derivative thereof. The sialic acid or derivative thereof can be naturally occurring or non-naturally occurring. In some cases, X comprises neuraminic acid, which is one example of a sialic acid, or a derivative thereof.

In some embodiments, the sialic acid is a naturally occurring sialic acid. The sialic acid family comprises approximately 50 naturally occurring members. Most common amongst these are N-acetylneuraminic acid (Neu5Ac), N-glycolylneuraminic acid (Neu5Gc, enzymatically produced from Neu5Ac by adding a single oxygen atom (i.e., hydroxylation)), 2-keto-3 deoxynonulosonic acid (Kdn), and neuraminic acid (Neu); others are well known in the art, as reviewed in, e.g. Schauer (2000) Glycoconjugate J 17:485-499. Thus, for example, when the Siglec to be targeted is CD22, the Siglec ligand may be a naturally occurring CD22 ligand, i.e. α2,6-linked sialic acid such as Neu5Aca2-6Galβ-4GlcNAc-6S; when the Siglec is Siglec-5, the Siglec ligand may be a naturally occurring Siglec-5 ligand, i.e. Neu5Aca8-8Neu5Ac and Neu5Aca2-6GalNAc; when the Siglec is Siglec-6, the Siglec ligand may be a naturally occurring Siglec-6 ligand, i.e. Neu5Aca2-6GalNAc; when the Siglec is Siglec-9, the Siglec ligand may be a naturally occurring Siglec-9 ligand, i.e. Neu5Acα2-3Galβ-4GlcNAc-65α-3fucose; or when the Siglec is Siglec-10/G, the Siglec ligand may be a naturally occurring Siglec-10 ligand, i.e. α2,6-linked sialic acid or α2,3-linked sialic acid, such as Neu5Acα2-6Galβ-4GlcNAc (O'Reilly, M. K. and Paulson, J. C. (2009) Trends Pharmacol. Sci. 30, 240-248). In some embodiments, the hypoimmunogenic biotherapeutic has been engineered to comprise the sialic acid ligands for two or more Siglecs, e.g. for 3 Siglecs or for 4 Siglecs, in certain cases, for 5 Siglecs. In some cases there are 5, 6, 7, 8, 9, 10, 11, 12, 13, or 15 Siglecs.

In some embodiments, the sialic acid is a non-naturally occurring, i.e. synthetic, sialic acid. A “synthetic sialic acid” also known in the art and referred to herein as a “sialic acid mimetic” or “SAM”, refers to a sialic acid that does not occur in nature, i.e. an alpha-keto acid sugar derivative comprising a nine-carbon backbone that is non-naturally occurring. Compared with Siglec ligands comprising natural sialic acids, which have weak monovalent binding affinities for Siglecs (0.1-3 mM), Siglec ligands comprising SAMs can feature binding affinities in the nanomolar range (Crocker, P. R. et al. (2007) Nat. Rev. Immunol. 7, 255-266; Prescher, H. et al. (2014) ACS Chem. Biol. 9, 1444-1450). SAMs that find use in the subject compositions include those in which one or more positions of a natural sialic acid ranging from the aglycone (C-2) to the rest of the backbone (C-3 to C-9) have been modified to improve Siglec binding. For example, the modifications C-9-NH2 (9-NH2-Neu5Ac/Me) and C-5-FAc (Neu5FAc/Me) improve Siglec-2 binding due to an increase in hydrogen bonding and lipophilic interactions between the SAM and Siglec-2, and incorporating a lipophilic group has since been used to rationally design additional SAMs having an increased binding affinity for Siglec-2 (van Rossenberg, S. M. W. et al. (2001) J. Biol. Chem. 276, 12967-12973; Kelm, S. et al. (2002) J. Exp. Med. 195, 1207-1213; Zaccai, N. R. et al. (2003) Structure 11, 557-567). Other nonlimiting examples of SAMs that find use in the present application include 9-N-biphenylcarboxyl-NeuAcα2-Galβ1-4GlcNAc (6′-BPCNeuAc), NeuAcα2-6Galβ1-4GlcNAc, NeuAcα2-6Galβ1-4(6-sulfo)GlcNAc; those SAMs disclosed in Bull et al. (2016) Sialic Acid Mimetics to Target the Sialic Acid-Siglec Axis. Trends Biochem Sci. 41(6):519-531 and Prescher, H. et al. (2014) Discovery of multifold modified sialosides as human CD22/Siglec-2 ligands with nanomolar activity on B-cells. ACS Chem. Biol. 9, 1444-1450; and those SAMs disclosed in U.S. Pat. Nos. 8,357,671, 9,522,183, 9,981,023, the full disclosures of which are incorporated herein by reference. In certain embodiments, the SAM is a SAM provided in Table 1 below.

TABLE 1

| Siglec | SAM | IC50 | | Reference |
|---|---|---|---|---|
| Siglec-2 (CD22) | 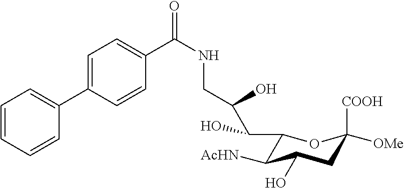 | 4 | μM | Kelm, S. et al. J. Exp. Med. 2002 |
| | 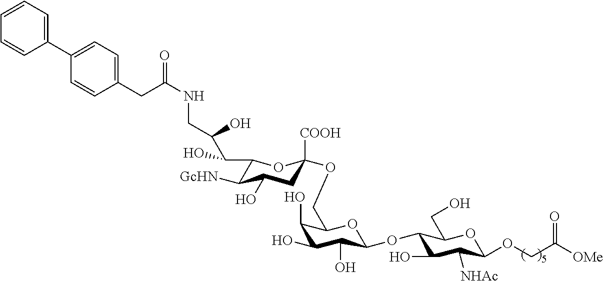 | 2.1 | μM | Collins, BE et al. J. Immunol 2006 |
| | 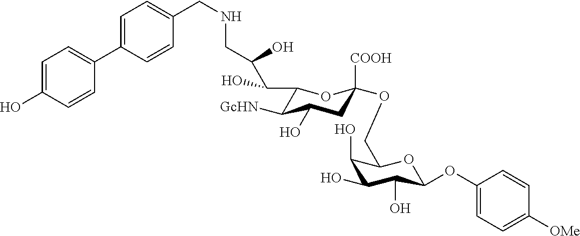 | 0.24 | μM | Abdu-Allah, HHM et al. J. Med Chem 2008 |
| | 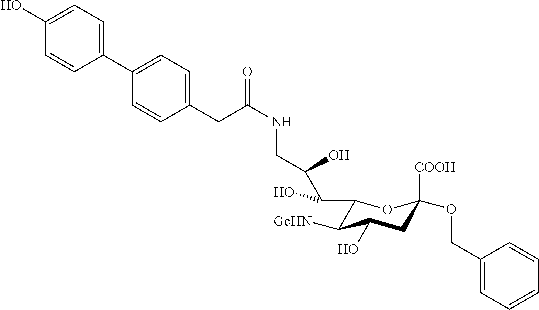 | 0.10 | μM | Abdu-Allah, HHM et al. Bioorg. Med. Chem Lett 2009; Abdu-Allah, HHM et al. Bioorg Med Chem 2011 |
| | 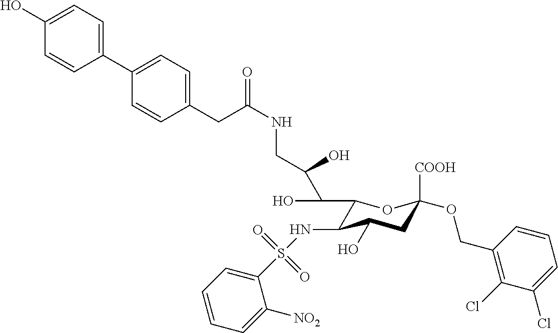 | 0.06 | μM | Mesch S et al. Chem Med Chem 2012 |
| | 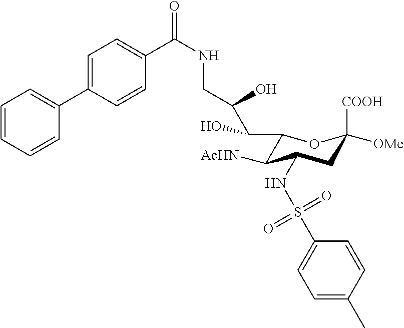 | 0.15 | μM | Kalm S. et al. Angew. Chem. Int. Ed. Engl 2013 |

| | | | |
|---|---|---|---|
| | 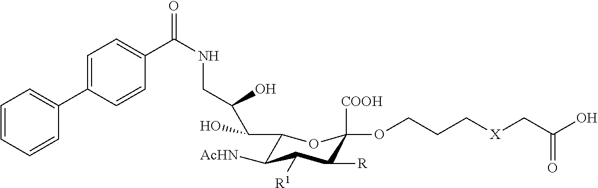 | 6 nM or 2 nM | Prescher, H. et al. ACS Chem. Biol. 2014 |
| | R = OR, R¹ = NH(CO)Et, X = CH₂, IC50= 6 nm | | |
| | R = H, R¹ = OSO₃Na, X = SCH₂, IC50= 2 nm [51] | | |
| Siglec-5 (CD170) | 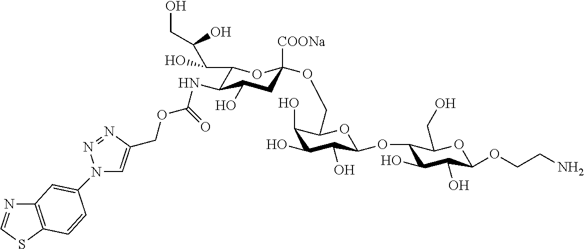 | NA | Rillahan, CD et al. Angew. Chem. Int. Ed. Engl 2012 |
| Siglec-9 (CD329) | 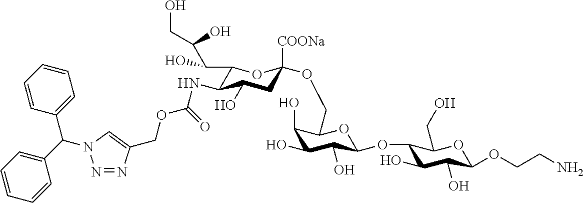 | NA | Rillahan, CD et al. Angew. Chem. Int. Ed. Engl 2012 |
| Siglec-10 (Siglec-G) | 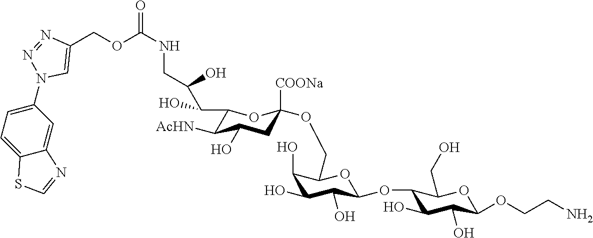 | NA | Rillahan, CD et al. Angew. Chem. Int. Ed. Engl 2012 |

As described in formula (I) above, n is an integer of 1 or more, such as an integer from 1 to 20, or from 1 to 15, or from 1 to 10, or from 1 to 5. In some cases, n is 1, 2, 3, 4, or 5. In some cases, n is 1. In some cases, n is 2. In some cases, n is 3. In some cases, n is 4. In some cases, n is 5.

If more than one X is present, i.e. if n is greater than 1, then the X groups can be the same or different from each other. If L is present, then each X is directly covalently bonded to L, and L is directly covalently bonded to Y. If L is absent, then each X is directly covalently bonded to Y. In some cases, n is 1. In some cases, n is an integer of 2 or more, such as 2, 3, 4, 5, 6, 7, 8, 9, or 10.

As stated above, m is an integer of 1 or more. In some cases, m is 2 or more, 3 or more, 5 or more, or 10 or more. In some cases, m ranges from 1 to 20, such as from 2 to 10.

### Linker L

L is an optional linker. As such, in some cases the engineered hypoimmunogenic biotherapeutic has the linker, and the engineered hypoimmunogenic biotherapeutic can be described by the formula [Xₙ-L]ₘ-Y. In other cases, the engineered hypoimmunogenic biotherapeutic does not have the linker, and the engineered biotherapeutic can be described by the formula [Xₙ]ₘ—Y.

Embodiments of the engineered hypoimmunogenic biotherapeutic can be used to demonstrate possible configurations of [Xₙ-L]ₘ-Y. As described above, X is a sialic acid group comprising a sialic acid or a derivative thereof. In the compound shown below, a single neuraminic acid group is present, corresponding to a single X group. Hence, in the embodiment below, n is 1. The group shown to the left of the neuraminic acid with the formula (phenyl)-C(O)-phenylene- can be considered to be a part of the X group. Although the biotherapeutic Y is not shown in the compound, the —C(O)—O—C₆F₅ group can undergo a chemical reaction that forms a covalent bond with a biotherapeutic Y.

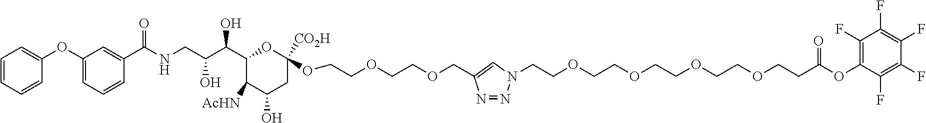 [SMILES: CC(=O)N[C@H]1[C@H]([C@H](O)[C@H](O)CNC(=O)c2cccc(Oc3ccccc3)c2)O[C@](OCCOCCOCc2cn(CCOCCOCCOCCOCCC(=O)Oc3c(F)c(F)c(F)c(F)c3F)nn2)(C(=O)O)C[C@@H]1O]

In the embodiment shown below, three neuraminic acid groups are shown, indicating that there are three X groups, and thus n is 3. In addition, the three X groups are covalently bonded to each other through a branching group comprising derivatives of lysine residues. This branching group is part of linker L. Stated in another manner, this embodiment includes the optional linker L, wherein L is a branching group that covalently connects the three X groups to one another. Linker L also covalently connects the X groups to the —C(O)—O—C₆F₅ group that can undergo a chemical reaction that forms a covalent bond with a biotherapeutic Y. Hence, linker L also covalently links the X groups to biotherapeutic Y.

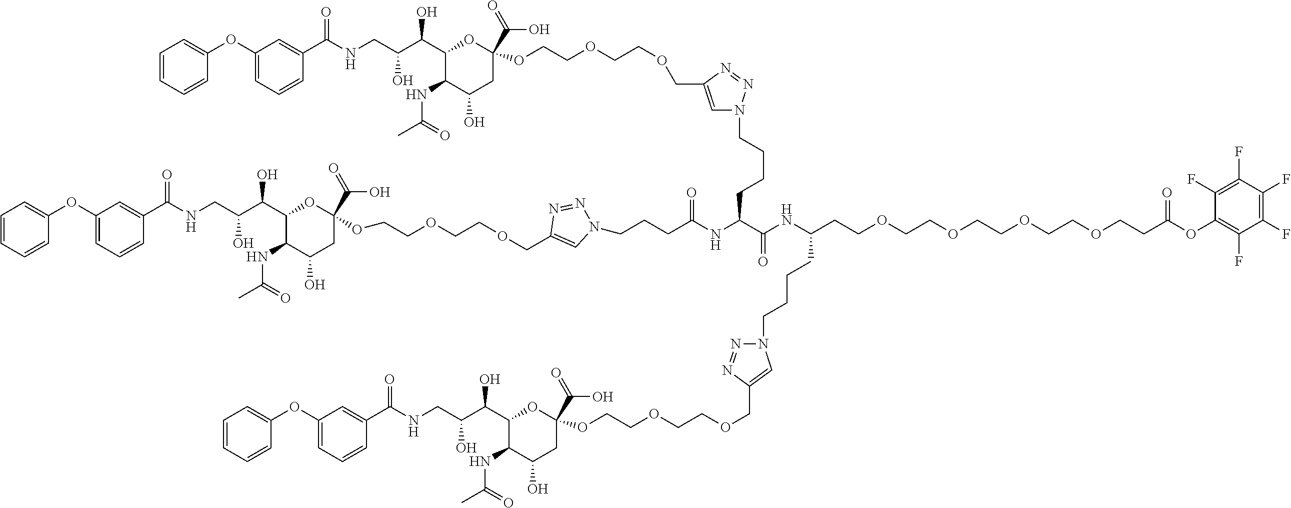 [SMILES: CC(=O)N[C@H]1[C@H]([C@H](O)[C@H](O)CNC(=O)c2cccc(Oc3ccccc3)c2)O[C@@](OCCOCCOCc2cn(CCCC[C@@H](CCOCCOCCOCCOCCC(=O)Oc3c(F)c(F)c(F)c(F)c3F)NC(=O)[C@H](CCCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)NC(=O)CCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)nn2)(C(=O)O)C[C@@H]1O]

As described above, the combination of X and L groups is collectively referred to as a Siglec ligand. In other words, [Xₙ-L] is a Siglec ligand.

In some embodiments, each sialic acid group of each X is covalently connected to biotherapeutic Y through a chain of atoms that does not include a sugar group. For instance, each X group includes a single sialic acid group but does not include any other sugar groups. Furthermore, in some embodiments, if L is present, L directly covalently connects each X to Y and L does not comprise a sugar group. The term “sugar” as used herein refers to monosaccharides and disaccharides. As such, even if L includes a trisaccharide, such a L would not be within the scope of such embodiments because a trisaccharide includes a disaccharide unit and a monosaccharide unit. Stated in another manner, the only sugar groups present in [Xₙ-L] is a single sialic acid group for each X. No sialic acid groups are directly covalently bonded to one another.

In some embodiments, each sialic acid group of each X is covalently connected to biotherapeutic Y through a chain of atoms that does not include an oxygen-containing heterocyclic group. Notably, monosaccharide sugars such as glucose and galactose are heterocyclic groups containing an oxygen atom in the ring. In some embodiments, each sialic acid group of each X is covalently connected to biotherapeutic Y through a chain of atoms that consists of one or more chemical moieties selected from the group consisting of: alkyl, alkenyl, alkynyl, polyethylene glycol, aryl, heteroaryl, sulfur atom-containing heterocycle, nitrogen atom-containing heterocycle, amino acid residue, amino, acyl, halo, hydroxy, carboxy, sulfoxy, and substituted analogs thereof.

In some embodiments, if present, linker L consists of one or more chemical moieties selected from the group consisting of: alkyl, alkenyl, alkynyl, polyethylene glycol, aryl, heteroaryl, sulfur atom-containing heterocycle, nitrogen atom-containing heterocycle, amino acid residue, amino, acyl, halo, hydroxy, carboxy, sulfoxy, and substituted analogs thereof. In addition, the section of X between the sialic acid and L or Y consists of one or more chemical moieties selected from the group consisting of: alkyl, alkenyl, alkynyl, polyethylene glycol, aryl, heteroaryl, sulfur atom-containing heterocycle, nitrogen atom-containing heterocycle, amino acid residue, amino, acyl, halo, hydroxy, carboxy, sulfoxy, and substituted analogs thereof.

In some cases wherein L is present, L is a branched linker. In other words, L directly covalently connects biotherapeutic Y to two or more X groups. In some cases, a branching location of L includes an amino acid residue or a derivative thereof, e.g. lysine or a derivative thereof. For instance, the branching location of L can have the formula shown below, wherein each location marked with an asterisk (*) is a site for heading towards an X group or the Y group.

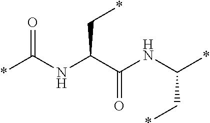 [SMILES: *C[C@@H](*)NC(=O)[C@H](C*)NC(*)=O]

In some cases, the branching location of L does not comprise an aryl group or a heteroaryl group. In some cases, the branching location of L comprises an alkyl group, an amide group, an amino acid residue group, or a combination thereof.

In some embodiments, linker L comprises a polyethylene glycol group, a triazole group, or a combination thereof. In some cases, the section of X between the sialic acid group and L or Y comprises a polyethylene glycol group, a triazole group, or a combination thereof. In some cases, the triazole group is part of a covalent connection between the X and L groups.

In some embodiments, the linker, L, can include one or more linker subunits (LS), such as 2, 3, 4, 5, 6, 7, 8, 9 or 10, or even more linker subunits (LS). For example, some embodiments of the linker can include 1 to 10 linker subunits (LS) described by Formula (II):

-(LS¹)ₐ-(LS²)_{b}-(LS³)_{c}-(LS⁴)_{d}-(LS⁵)ₑ-(LS⁶)_{f}-(LS⁷)_{g}-(LS⁸)ₕ-(LS⁹)ᵢ-(LS¹⁰)ⱼ- (II)

where LS¹, LS², LS³, LS⁴, LS⁵, LS⁶, LS⁷, LS⁸, LS⁹ and LS¹⁰ are each independently a linker subunit, and a, b, c, d, e, f, g, h, i and j are each independently 0 or 1. In some embodiments, the sum of a to j is 1 (e.g., a is 1 and b to j are each 0). In these embodiments, the linker subunit LS¹ is attached at one end to Y and at the other end to X. In some embodiments, the sum of a to j is 2 (e.g., a and b are each 1, and c to j are each 0). In these embodiments, the linker subunit LS¹ is attached to Y and the linker subunit LS² is attached to X. In some embodiments, the sum of a to j is 3 (e.g., a to c are each 1, and d to j are each 0). In these embodiments, the linker subunit LS¹ is attached to Y and the linker subunit LS³ is attached to X. In some embodiments, the sum of a to j is 4 (e.g., a to d are each 1, and e to j are each 0). In these embodiments, the linker subunit LS¹ is attached to Y and the linker subunit LS⁴ is attached to X. In some embodiments, the sum of a to j is 5 (e.g., a to e are each 1, and f to j are each 0). In these embodiments, the linker subunit LS¹ is attached to Y and the linker subunit LS¹ is attached to X. In some embodiments, the sum of a to j is 6 (e.g., a to f are each 1, and g to j are each 0). In these embodiments, the linker subunit LS¹ is attached to Y and the linker subunit LS⁶ is attached to X. In some embodiments, the sum of a to j is 7 (e.g., a to g are each 1, and h to j are each 0). In these embodiments, the linker subunit LS¹ is attached to Y and the linker subunit LS⁷ is attached to X. In some embodiments, the sum of a to j is 8 (e.g., a to h are each 1, and i and j are each 0). In these embodiments, the linker subunit LS¹ is attached to Y and the linker subunit LS⁹ is attached to X. In some embodiments, the sum of a to j is 9 (e.g., a to i are each 1, and j is 0). In these embodiments, the linker subunit LS¹ is attached to Y and the linker subunit LS⁹ is attached to X. In some embodiments, the sum of a to j is 10 (e.g., a to j are each 1). In these embodiments, the linker subunit LS¹ is attached to Y and the linker subunit LS¹⁰ is attached to X.

Any convenient functional group can be used in each linker subunit (LS) in the linker. In some embodiments, a linker subunit (LS) may include a group selected from, but not limited to, alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl.

In some embodiments, a linker subunit includes a functional group independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, substituted heterocyclyl, (PEG)ₙ, and (AA)ₚ, where each n is independently an integer from 1 to 50 and each p is independently an integer from 1 to 20. As used herein, “PEG” refers to polyethylene glycol.

As used herein, “AA” refers to an amino acid residue. Amino acid residues include amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). In some embodiments, amino acid residues used in the linkers and linker subunits described herein also include amino acid analogs and amino acid derivatives, which are natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or attachment of another group to the side chain or backbone, or combinations thereof. For example, amino acid analogs may include α-hydroxy acids, and α-amino acids, and the like. In some instances, an amino acid analog or amino acid derivative can include another group, such as another sialic acid moiety (X), attached to the side chain or backbone of the amino acid analog or amino acid derivative through an optional linker.

In some embodiments, a linker subunit includes a functional group independently selected from (C₁-C₁₂)alkyl or substituted (C₁-C₁₂)alkyl. In some embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₁-alkylene (e.g., CH₂). In some instances, (C₁-C₁₂)alkyl is a C₂-alkylene (e.g., CH₂CH₂).

In some embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₁-alkylene. In some instances, substituted (C₁-C₁₂)alkyl is a substituted C₂-alkylene.

In some embodiments, a linker subunit includes a functional group independently selected from aryl, substituted aryl, heteroaryl, substituted heteroaryl, cycloalkyl, substituted cycloalkyl, heterocyclyl, and substituted heterocyclyl.

In some instances, a linker subunit includes a functional group independently selected from aryl or substituted aryl. In some instances, the linker subunit includes an aryl. For example, the aryl can be phenyl. In some instances, the linker subunit includes a substituted aryl. In some cases, the substituted aryl is a substituted phenyl. The substituted aryl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl.

In some instances, a linker subunit includes a functional group independently selected from heteroaryl or substituted heteroaryl. In some cases, the linker subunit includes a heteroaryl. In some cases, the linker subunit includes a substituted heteroaryl. The substituted heteroaryl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl.

In some instances, a linker subunit includes a functional group independently selected from cycloalkyl or substituted cycloalkyl. In some cases, the linker subunit includes a cycloalkyl. In some cases, the linker subunit includes a substituted cycloalkyl. The substituted cycloalkyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl.

In some instances, a linker subunit includes a functional group independently selected from heterocyclyl or substituted heterocyclyl. In some cases, the linker subunit includes a heterocycloalkyl. For example, the linker subunit can include a triazole (e.g., 1,2,3-triazole). In some cases, the linker subunit includes a substituted heterocycloalkyl. The substituted heterocyclyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl.

In some embodiments, the linker does not include a natural saccharide.

In some embodiments, the linker (L) includes one or more tether groups adjacent to or in between one or more linker subunits (LS) in the linker. The tether groups may facilitate attachment between two linker subunits, between a linker subunit and a reactive termini for conjugation to the moiety of interest (Y), or between a linker subunit and the sialic acid moiety (X). The tether groups may include convenient functional groups that facilitate these attachments, such as, but not limited to, amino, carbonyl, amido, oxycarbonyl, carboxy, thioether, sulfonyl, sulfoxide, sulfonylamino, aminosulfonyl, thio, oxy, phospho, phosphoramidate, thiophosphoramidate, and the like. In some embodiments, the tether groups are each independently selected from a covalent bond, —CO—, —NR¹⁵—, —NR¹⁵(CH₂)_{q}—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In some embodiments, q is an integer from 1 to 6 (e.g., 1, 2, 3, 4, 5 or 6). In some embodiments, q is 1. In some embodiments, q is 2. In some embodiments, q is 3. In some embodiments, q is 4. In some embodiments, q is 5. In some embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In some embodiments, R¹⁵ is hydrogen. In some embodiments, each R¹⁵ is hydrogen. In some embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In some embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In some embodiments, R¹⁵ is alkynyl or substituted alkynyl. In some embodiments, R¹⁵ is alkoxy or substituted alkoxy. In some embodiments, R¹⁵ is amino or substituted amino. In some embodiments, R¹⁵ is carboxyl or carboxyl ester. In some embodiments, R¹⁵ is acyl or acyloxy. In some embodiments, R¹⁵ is acyl amino or amino acyl. In some embodiments, R¹⁵ is alkylamide or substituted alkylamide. In some embodiments, R¹⁵ is sulfonyl. In some embodiments, R¹⁵ is thioalkoxy or substituted thioalkoxy. In some embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In some embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In some embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In some embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In some embodiments, a linker subunit (LS) may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In some embodiments, the polymer is a polyalkylene glycol. In some embodiments, the polymer is a polyethylene glycol (PEG).

In some cases, the linker is not branched and connects one X group to the Y group, and thus may be referred to as monovalent. In some cases, the linker is a branched linker that is divalent and connects two X groups to the Y group. In certain cases, the linker is a branched linker that is trivalent and connects three X groups to the Y group. In some instances, the linker is a branched linker of a higher multivalency and connects multiple X groups to the Y group. In some cases, the linker has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a linker subunit (LS) may be a branched subunit. A branched subunit may be a linker subunit that is attached to two or more sialic acid moieties, either directly or through a respective linker for each sialic acid moiety. For example, a branched subunit may be attached to two sialic acid moieties. In some embodiments, a branched subunit includes an amino acid (AA). For instance, a branched subunit may include an amino acid where the backbone of the amino acid forms part of the linker attached to a first sialic acid moiety and where the side chain of the amino acid is conjugated to a second sialic acid moiety either directly or through a linker of the branch (i.e., “a branch linker”). For example, in some embodiments, a branched subunit includes a lysine where the backbone of the lysine forms part of the linker attached to a first sialic acid moiety and where the side chain of the lysine is conjugated to a second sialic acid moiety either directly or through a branch linker. In some embodiments, the second sialic acid moiety is conjugated to the lysine by attachment at the terminal amine of the lysine side chain. In some embodiments, the branch linker is a linker as described by Formula (II) above.

Examples of linkers according to the present disclosure include, but are not limited to, the following:
LS¹ is PEG (e.g., PEG₄); LS² is heterocyclyl (e.g., 1,2,3-triazole); LS³ is PEG (e.g., PEG₂); and d to j are each 0;
LS¹ is PEG (e.g., PEG₄); LS² is a branched subunit (e.g., lysine); LS³ is alkyl (e.g., C₃-alkyl); LS⁴ is PEG (e.g., PEG₂); LS¹ is heterocyclyl (e.g., 1,2,3-triazole); LS⁶ is PEG (e.g., PEG₂); and g to j are each 0; and where the LS² branched subunit is attached to a branch linker described by the following: LS¹ is PEG (e.g., PEG₂); LS² is heterocyclyl (e.g., 1,2,3-triazole); LS³ is PEG (e.g., PEG₂); and d to j are each 0;
LS¹ is PEG (e.g., PEG₄); LS² is heterocyclyl (e.g., 1,2,3-triazole); LS³ is alkyl (e.g., C₂-alkyl); and d to j are each 0;
LS¹ is PEG (e.g., PEG₄); LS² is a branched subunit (e.g., lysine); LS³ is alkyl (e.g., C₃-alkyl); LS⁴ is PEG (e.g., PEG₂); LS¹ is heterocyclyl (e.g., 1,2,3-triazole); LS⁶ is alkyl (e.g., C₂-alkyl); and g to j are each 0; and where the LS² branched subunit is attached to a branch linker described by the following: LS' is PEG (e.g., PEG₂); LS² is heterocyclyl (e.g., 1,2,3-triazole); LS³ is alkyl (e.g., C₂-alkyl); and d to j are each 0;
LS¹ is PEG (e.g., PEG₄); LS² is a branched subunit (e.g., lysine); LS³ is a branched subunit (e.g., lysine); LS⁴ is alkyl (e.g., C₃-alkyl); LS¹ is PEG (e.g., PEG₂); LS⁶ is heterocyclyl (e.g., 1,2,3-triazole); LS⁷ is alkyl (e.g., C₂-alkyl); and h to j are each 0; where the LS² branched subunit is attached to a branch linker described by the following: LS¹ is PEG (e.g., PEG₂); LS² is heterocyclyl (e.g., 1,2,3-triazole); LS³ is alkyl (e.g., C₂-alkyl); and d to j are each 0; and where the LS³ branched subunit is attached to a branch linker described by the following: LS¹ is PEG (e.g., PEG₂); LS² is heterocyclyl (e.g., 1,2,3-triazole); LS³ is alkyl (e.g., C₂-alkyl); and d to j are each 0;
LS¹ is PEG (e.g., PEG₄); LS² is a branched subunit (e.g., lysine); LS³ is a branched subunit (e.g., lysine); LS⁴ is alkyl (e.g., C₃-alkyl); LS¹ is PEG (e.g., PEG₂); LS⁶ is heterocyclyl (e.g., 1,2,3-triazole); LS⁷ is PEG (e.g., PEG₂); and h to j are each 0; where the LS² branched subunit is attached to a branch linker described by the following: LS¹ is PEG (e.g., PEG₂); LS² is heterocyclyl (e.g., 1,2,3-triazole); LS³ is PEG (e.g., PEG₂); and d to j are each 0; and where the LS³ branched subunit is attached to a branch linker described by the following: LS¹ is PEG (e.g., PEG₂); LS² is heterocyclyl (e.g., 1,2,3-triazole); LS³ is PEG (e.g., PEG₂); and d to j are each 0;
LS¹ is a branched subunit (e.g., lysine); LS² is a branched subunit (e.g., lysine); LS³ is alkyl (e.g., C₃-alkyl); LS⁴ is heterocyclyl (e.g., 1,2,3-triazole); LS¹ is PEG (e.g., PEG₂); and f to j are each 0; where the LS¹ branched subunit is attached to a branch linker described by the following: LS¹ is heterocyclyl (e.g., 1,2,3-triazole); LS² is PEG (e.g., PEG₂); and c to j are each 0; and where the LS² branched subunit is attached to a branch linker described by the following: LS¹ is heterocyclyl (e.g., 1,2,3-triazole); LS² is PEG (e.g., PEG₂); and c to j are each 0;
LS¹ is PEG (e.g., PEG₄); LS² is a branched subunit (e.g., lysine); LS³ is a branched subunit (e.g., lysine); LS⁴ is alkyl (e.g., C₃-alkyl); LS¹ is heterocyclyl (e.g., 1,2,3-triazole); LS⁶ is PEG (e.g., PEG₂); and g to j are each 0; where the LS² branched subunit is attached to a branch linker described by the following: LS¹ is heterocyclyl (e.g., 1,2,3-triazole); LS² is PEG (e.g., PEG₂); and c to j are each 0; and where the LS³ branched subunit is attached to a branch linker described by the following: LS¹ is heterocyclyl (e.g., 1,2,3-triazole); LS² is PEG (e.g., PEG₂); and c to j are each 0; and
LS¹ is PEG (e.g., PEG₄); LS² is a branched subunit (e.g., lysine); LS³ is alkyl (e.g., C₃-alkyl); LS⁴ is heterocyclyl (e.g., 1,2,3-triazole); LS¹ is PEG (e.g., PEG₂); and f to j are each 0; and where the LS² branched subunit is attached to a branch linker described by the following: LS¹ is heterocyclyl (e.g., 1,2,3-triazole); LS³ is PEG (e.g., PEG₂); and c to j are each 0.

The linkers described above may include one or more tether groups to facilitate attachment between two linker subunits, between a linker subunit and a reactive termini for conjugation to the moiety of interest (Y), or between a linker subunit and the sialic acid moiety (X).

Siglec Ligand [Xₙ-L]ₘ

In the formula [Xₙ-L]ₘ-Y, [Xₙ-L] represent the Siglec ligand, and m represent an integer from 1-25. It is conceived that one or more Siglec ligands, e.g. 2, 3, 4, or 5 or more Siglec ligand, in some cases 6, 7, 8, 9, or 10 or more Siglec ligands, in some such cases 11, 12, 13, 14, 15 or more Siglec ligands, in some cases 16, 17, 18, 19, 20 or more Siglec ligands, sometimes 21, 22, 23, 24 or 25 Siglec ligands, may be conjugated to the biotherapeutic, either appended to the same or to different amino acids of the biotherapeutic. The Siglec ligand can be naturally occurring, i.e. a moiety comprising a naturally occurring sialic acid and a naturally occurring glycan, wherein the sialic acid and glycan are typically found in nature in association with one another to form a Siglec ligand. The Siglec ligand can be non-naturally occurring, e.g. a moiety comprising a naturally occurring sialic acid and a linker, a moiety comprising a non-naturally occurring sialic acid and a glycan found in nature as part of a Siglec ligand, a moiety comprising a non-naturally occurring sialic acid and a linker, a moiety comprising a peptide having an affinity for a Siglec, and the like.

### Biotherapeutic Y

In the formula [Xₙ-L]ₘ-Y, Y is the biotherapeutic. Y by itself is referred to as an unengineered biotherapeutic, which term is used interchangeably with the term parental biotherapeutic. However, the combination of elements that is [Xₙ-L]ₘ-Y is referred to as the engineered hypoimmunogenic biotherapeutic. Y, in and of itself, has a therapeutic activity. [Xₙ-L] does not mediate the therapeutic activity of Y. Stated in another manner, the therapeutic activity of Y is independent of the presence or absence of [Xₙ-L].

Exemplary biotherapeutic Y groups include antibodies, enzymes, viral particles, nanoparticles, polypeptides, and nucleic acids.

Any protein or nucleic acid biotherapeutic may serve as the biotherapeutic that is engineered to become a hypoimmunogenic biotherapeutic according to the present disclosure, including, for example, a protein, e.g. an antibody, a fusion protein, an enzyme, a viral particle, a DNA molecule or an RNA molecule. The biotherapeutic may be naturally occurring, for example a naturally occurring protein that is delivered to a patient as a therapeutic, a naturally occurring capsid, etc. The biotherapeutic may be an engineered protein, for example, an antibody therapeutic, a fusion or “chimeric” protein, i.e. a protein comprising protein domains from two or more different proteins, or an entirely non-natural protein, i.e. having 30% identity or less with any naturally occurring protein across its functional domains (see e.g. Chen et al. (2020) De novo design of protein logic gates. Science 368 (6486): 78-84; and Polizzi et al. (2020) A defined structural unit enables de novo design of small-molecule-binding proteins Science 369 (6508): 1227-1233). In some embodiments, the biotherapeutic is a variant of a naturally occurring protein or a known engineered protein. By “variant” it is meant a mutant of a protein having less than 100% sequence identity with the protein from which it is derived. For example, a variant protein may be a protein having 60% sequence identity or more with a full length native protein, e.g. 65%, 70%, 75%, or 80% or more identity, such as 85%, 90%, or 95% or more identity, for example, 98% or 99% identity with the full length native protein. Variants also include fragments of naturally occurring proteins, particularly those having comparable or improved activity over the naturally occurring protein. The biotherapeutic may be derived from any source, e.g. human, non-human, or engineered.

In some embodiments, the protein is an antibody or fragment thereof, for example a monoclonal antibody, a bispecific antibody, a trispecific antibody, an scFv, a Fab, a camelid nanobody, etc. Nonlimiting examples of antibodies for which the engineering contemplated herein finds particular use include adalimumab and infliximab (for the treatment of autoimmune or an inflammatory disease such as rheumatoid arthritis, psoriatic arthritis, ankylosing spondylitis, Crohn's disease, ulcerative colitis, psoriasis, hidradenitis suppurativa, uveitis, or juvenile idiopathic arthritis), cetuximab (for the treatment of cancers, including for example metastatic colorectal cancer, metastatic non-small cell lung cancer and head and neck cancer), natalizumab (for the treatment of multiple sclerosis), Lumoxiti/moxetumomab pasudotox (for the treatment of hairy cell leukemia), Tecentriq/atezolizumab (for the treatment of various cancers), Opdivo/Nivolumab (for the treatment of various cancers), Reopro/abciximab (anti-GPIIb/IIIa, for the prevention of thrombosis during and after coronary artery procedures such as angioplasty), Brentuximab (for the treatment of relapsed or refractory Hodgkin lymphoma (HL) and systemic anaplastic large cell lymphoma (ALCL)), Certolizumab pegol (for the treatment of Crohn's disease, rheumatoid arthritis, psoriatic arthritis and ankylosing spondylitis), Elotuzumab (for the treatment of relapsed multiple myeloma), Benralizumab (for the treatment of asthma), Vedolizumab (for the treatment of ulcerative colitis and Crohn's disease), Galcanezumab (for the treatment of migraines and cluster headaches), Rituximab (for the treatment of autoimmune diseases and various cancer), Alemtuzumab (for the treatment of chronic lymphocytic leukemia (CLL) and multiple sclerosis), Dupilumab (for the treatment of allergic diseases such as eczema (atopic dermatitis), asthma and nasal polyps), Golimumab (for the treatment of inflammation), Obinutuzumab (for the treatment of lymphomas, e.g. chronic lymphocytic leukemia, follicular lymphoma), Tildrakizumab (for the treatment of immunologically mediated inflammatory disorders), Erenumab (for the prevention of migraine), Mepolizumab (for the treatment of severe eosinophilic asthma, eosinophilic granulomatosis, and hypereosinophilic syndrome (HES)), Ramucirumab (for the treatment of solid tumors), Ranibizumab (for the treatment of “wet” age-related macular degeneration (AMD, also ARMD), diabetic retinopathy, and macular edema), Ustekinumab (for the treatment of psoriasis, Crohn's disease, and ulcerative colitis), Reslizumab (for the treatment of asthma), Ipilimumab (for the treatment of various cancers), Alirocumab (for the treatment of high cholesterol), Belimumab (for the treatment of systemic lupus erythematosus (SLE)), Panitumumab (for the treatment of various cancers), Avelumab (for the treatment of Merkel cell carcinoma, urothelial carcinoma, and renal cell carcinoma), Necitumumab (for the treatment of metastatic squamous non-small-cell lung carcinoma (NSCLC)), Mogamulizumab (for the treatment of relapsed or refractory mycosis fungoides and Sezary disease, relapsed or refractory CCR4+ adult T-cell leukemia/lymphoma (ATCLL), and relapsed or refractory CCR4+ cutaneous T cell lymphoma (CTCL)), Olaratumab (for the treatment of solid tumors), Brodalumab (for the treatment of inflammatory diseases), Eculizumab (for the treatment of paroxysmal nocturnal hemoglobinuria (PNH), atypical hemolytic uremic syndrome (aHUS), and neuromyelitis optica), Pertuzumab (for the treatment of metastatic HER2-positive breast cancer), Pembrolizumab (for the treatment of various cancers), and Tocilizumab (for the treatment of rheumatoid arthritis (RA) and systemic juvenile idiopathic arthritis).

In some embodiments, biotherapeutic Y is an antibody that is not an antibody that is specific for a receptor selected from a B cell receptor (BCR), a receptor for the Fc region of immunoglobulin E (FcIRI), a Toll Like receptor (TLR), a T-cell receptor (TCR), or complexes thereof.

As used herein, an antibody that specifically binds to a target antigen refers to an antibody comprising a complementarity determining region (CDR) domain that specifically recognizes and binds to the target antigen. Thus, an antibody that specifically binds to a B cell receptor or complex thereof refers to an antibody comprising a CDR that specifically recognizes and binds to a B cell receptor or a complex comprising a B cell receptor, an antibody that specifically binds to a receptor for the Fc region of IgE refers to an antibody comprising a CDR that specifically recognizes and binds to a receptor for the Fc region of IgE or a complex comprising a receptor for the Fc region of IgE, an antibody that specifically binds to a Toll like receptor refers to an antibody comprising a CDR that specifically recognizes and binds to a Toll-like receptor or a complex comprising a Toll-like receptor, and an antibody that specifically binds to a T-cell receptor refers to an antibody comprising a CDR that specifically recognizes and binds to a T-cell receptor or a complex comprising a T-cell receptor

In some embodiments, the protein is a native, or naturally occurring, protein. In other embodiments, the protein is an engineered protein. Examples of proteins for which the engineering contemplated herein finds particular use include erythropoietin (EPO, to stimulate the production of red blood cells), thrombopoietin (TPO, to stimulate the production of platelets), human growth hormone, tissue factor, IFNβ-1b (for the treatment of Multiple Sclerosis), IFNβ-1a (for the treatment of Multiple Sclerosis), IL-2 or the IL-2 mimetic aldesleukin (for the treatment of melanoma and renal cell carcinoma), exenatide (for the treatment of Type 2 Diabetes), albiglutide (for the treatment of Type 2 Diabetes), alefacept (to control inflammation in moderate to severe psoriasis with plaque formation, for the treatment of cutaneous T-cell lymphoma and T-cell non-Hodgkin lymphoma), palifermin (to stimulate the growth of cells that line the surface of the mouth and intestinal tract following chemotherapy), belatacept (to promote graft/transplant survival), and neutral and basic amino acid transport protein rBAT or b(0,+)-type amino acid transporter 1 (for the treatment of cystinuria).

In some embodiments, biotherapeutic Y is a protein that is not ovalbumin or immunoglobulin E (IgE).

In some embodiments, the protein is an enzyme, for example a metabolic enzyme, a lysosomal enzyme, a protease, a peptidase, etc. Nonlimiting examples of enzymes for which the engineering contemplated herein finds particular use include asparaginase from Erwinia chrysanthemi (for the treatment of leukemia), bacterial IdeS (for immunosuppression following tissue transplantation or in the administration of a therapy, e.g. a gene therapy, for which the patient had preexisting immunity; for treatment of IgG antibody-driven diseases, such as Systemic lupus erythematosus, Pemphigus vulgaris or IgA Nephropathy), bacterial mucinase (for the treatment of MUC+ cancers, e.g. MUC1+ cancers), Factor VIII (for the treatment of Hemophilia A), Factor IX (for the treatment of Hemophilia B), Factor Xa (to promote clotting), a complement degrading protease, e.g. from a pathogen such as a bacterial pathogen or fungal pathogen (e.g. Pseudomonas Elastase (PaE), Pseudomonas Alkaline protease (PaAP), Streptococcal pyrogenic Exotoxin B (SpeB), a gingipain from Porphyromonas gingivalis, Aspergillus Alkaline protease 1 (Alp1), C. albicans Secreted aspartyl proteinases 1 (Sap1), 2 (Sap2), and 3 (Sap3) for the treatment of complement-mediated disease, such as IgA nephropathy), phenylalanine ammonia-lyase or the mimetic pegvaliase (for the treatment of PKU), alpha-galactosidase A (for the treatment of Fabry Disease), acid α-glucosidase or the mimetic Alglucosidase alfa (GAA, for the treatment of Pompe Disease), glucocerebrosidase (GCase, for the treatment of Gaucher), aspartylglucosaminidase (AGA, for the treatment of Aspartylglucosaminuria), asfotase (for treatment of hypophosphatasia (HPP)), alpha-L-iduronidase (for the treatment of MPS I), iduronate sulfatase or the iduronate sulfatase mimetic idursulfase (for the treatment of MPS II), sulfaminase (for the treatment of MPS Ilila), α-N-acetylglucosaminidase (NAGLU, for the treatment of MPS IIIB), heparin acetyle CoA: α-glucosaminide N-acetyltransferase (HGSNAT, for the treatment of MPS IIIC), N-acetylglucosamine 6-sulfatase (GNS, for the treatment of MPS IID), N-glucosamine 3-O-sulfatase (arylsulfatase G or ARSG, for the treatment of MPS IIIE), N-acetylgalactosamine 6-sulfatase (for the treatment of MPS IVA), beta-galactosidase (for the treatment of MPS IVB), N-acetylgalactosamine 4-sulfatase (for the treatment of MPS VI), beta-glucuronidase (for the treatment of MPS VI), palmitoyl protein thioesterase (PPT1, for the treatment of Batten disease/CLN1), Tripeptidyl peptidase (TPP1, for the treatment of Batten Disease/CLN2), arginase-1 or pegzilarginase (for the treatment of arginase-1 deficiency), or cystathionine beta synthase or Aeglea product AGLE-177 (for the treatment of cystathionine beta synthase (CBS) deficiency, also known as Classical Homocystinuria).

In some embodiments, biotherapeutic Y is an enzyme that is not Factor VIII.

In some embodiments, the protein is a viral protein or a viral particle, for example, a recombinant viral particle. By a “recombinant” virus or viral particle it is meant a virus/viral particle that comprises a genome comprising a polynucleotide that is heterologous to the virus, i.e., not found in nature to be associated with the capsid/envelope of the virus, wherein the polynucleotide encodes a gene product (RNA or protein). Recombinant viral particles find use in the delivery of polynucleotides that encode a therapeutic gene product for the purpose of gene therapy or oncolytic virus therapy. Gene therapy is a well-established art. Also well-established is the fact that gene therapy is severely hampered by the inability to readminister the same viral therapeutic more than once or a few times, owing to the fact that the viral particle will induce an immune response in an individual. As such, the ordinarily skilled artisan will appreciate that any viral particle used in gene therapy would benefit from engineering as contemplated herein. Nonlimiting examples of viral particles that may serve as the biotherapeutic that is engineered to become a hypoimmunogenic biotherapeutic according to the present disclosure include recombinant adeno-associated virus (rAAV) particles, e.g. an rAAV particle comprising a capsid VP1 protein from the group consisting of an AAV1, AAV2, AAV3b, AAV4, AAV5, AAV6, AAV7, AAV8, AAV9, AAVrh10, AAV11, AAV12, or AAV13 VP1 protein or a variant or pseudotyped virus thereof; recombinant human adenovirus particles, e.g. an rHAdV particle comprising a capsid protein from rHAdV-A, rHAdV-B, rHAdV-C, rHAdV-D, rHAdV-E, rHAdV-F, or rHAdV-G or a variant thereof; recombinant Herpes Simplex Virus (rHSV) particles, e.g. a rHSV1 or rHSV2 or variant or pseudotyped virus thereof; recombinant papillomavirus (PV) particles; recombinant polyomavirus particles; recombinant vaccinia virus particles; a recombinant cytomegalovirus (CMV) particle; a recombinant baculovirus particle; a recombinant human papillomavirus (HPV) particle; or a recombinant retrovirus particle, e.g. a recombinant lentivirus, recombinant human immunodeficiency virus (HIV) particle, Simian immunodeficiency virus (SIV) particle, Feline immunodeficiency virus (FIV) particle, Puma lentivirus (PLV) particle, Equine infectious anemia virus (EIAV) particle, Bovine immunodeficiency virus (BIV) particle, Caprine arthritis encephalitis virus particle, gammaretrovirus particle, and murine leukemia virus (MLV) particle, or variant or pseudotyped virus thereof.

In some embodiments, biotherapeutic Y is not a toxin. Generally, toxins are compounds that are harmful to cells in generally non-specific manner, i.e. a toxin will cause a similar amount of harm to different cells, even if such cells are from significantly different categories. In contrast, selectively damaging compounds will harm certain cells to a significantly greater degree than the harm inflicted on other types of cells. For instance, the selectively damaging compound can cause harm based on a biochemical process that is common in a lung cell but rare in a kidney cell, whereas a toxin can cause harm based on a biochemical process common to both lung and kidney cells. In some cases, the harm is cell death. In some cases, the toxin is Pseudomonas exotoxin A. In some embodiments, biotherapeutic Y is not a B cell modulator. Y does not increase or decrease the immune action of a B cell. Examples of modulation of the B cell include differentiation of the B cell into a biotherapeutic-specific mature B cell, e.g. plasma cells or memory cells, preventing B cells from producing antigen-specific antibodies, preventing the upregulation of activation markers such as CD69, promoting a decrease in viability of a biotherapeutic-specific B cell population. In some embodiments, B cell activation is inhibited only for those B cells with a B cell receptor that recognizes Y (in contrast to the entire B cell population recognizing X).

As discussed above, unlike a parental biotherapeutic that has not been engineered to comprise an altered Siglec ligand profile, the hypoimmunogenic biotherapeutic compositions of the present disclose will suppress the development of an immune response to themselves. As such, an engineered hypoimmunogenic biotherapeutic of the present disclosure may be functionally distinguished from the unengineered, i.e parental, biotherapeutic from which it is derived by assessing the extent to which the engineered hypoimmunogenic biotherapeutic attenuates the activity of immune cells. By attenuating an activity, it is meant slowing an increase in activity, reducing the activity, or preventing the activity, e.g. by silencing, inhibiting, deleting, etc. the cell or population of cells. Thus, for example, attenuating the activity of a B cell or a population of B cells may comprise preventing B cells from differentiating into biotherapeutic-specific mature B cells, e.g. plasma cells or memory cells, preventing B cells from producing antigen-specific antibodies, preventing the upregulation of activation markers such as CD69, promoting a decrease in viability of a biotherapeutic-specific B cell population. Typically, the therapeutic activity of the unengineered, i.e. parental, biotherapeutic does not comprise attenuating the activity of an immune cell. More typically, the therapeutic activity of the unengineered, i.e. parental, biotherapeutic does not comprise attenuating the activity of a B cell or a population of B cells.

The ability of a biotherapeutic engineered according to the present disclosure to suppress an immune response can be readily measured in any number of ways in vitro or in vivo. In vitro, immunosuppression can be measured as, for example, the extent to which a population of B cells is activated by the biotherapeutic, where less activation is indicative of greater immunosuppression. Any approach known in the art for measuring B cell activation may be used. For example, the extent to which the cells of the population upregulate CD69 expression when contacted with the engineered biotherapeutic can be assessed, e.g. by measuring the percent of CD69+ cells by FACS, by assessing the mean fluorescence intensity (MFI) of the B cells, by assessing the activity of the B cells, and the like. In such analyses it is expected that the engineered biotherapeutic will activate B cells at least about 2.5-fold less robustly than an unengineered biotherapeutic, in some instances at least about 5-fold less robustly, at least about 7.5-fold less robustly, or at least about 10-fold less robustly, in some instances about 20-fold less robustly. In vivo, immunosuppression may be measured as, for example, the extent to which the engineered biotherapeutic elicits “anti-drug antibodies”, or ADAs, relative to the ADAs elicited in an individual, e.g. a mouse injected intramuscularly or intravenously, e.g. in the presence or absence of an immunological adjuvant such as Alum, or, e.g. a human, upon administration of the corresponding unmodified biotherapeutic, where less ADAs is indicative of greater immunosuppression. In some instances, the ADA titer to the hypoimmunogenic biotherapeutic is reduced by 50% or more relative to the corresponding unmodified biotherapeutic, for example 60%, 70%, 80% or more, in certain instances 85%, 90%, 95% or more, preferably 98%, 99%, or 100%, i.e. so as to be undetectable. Put another way, the ADA titer that is elicited by the hypoimmunogenic biotherapeutic is 50% of that which is elicited by a corresponding unengineered biotherapeutic or less, for example, 40%, 30%, or 20% or less, in certain instances, 15%, 10%, 5% or less, preferably only 2%, 1% or less of that which is elicited by a corresponding unengineered biotherapeutic.

Methods for detecting antibodies, including but not limited to enzyme-linked immunosorbent assay (ELISA), microparticle ELISA, ELISPOT, radio-immunoprecipitation assays, Electrochemiluminescence immunoassay (ECLIA), DELFIA (dissociation-enhanced lanthanide fluorescence immunoassay) Time-Resolved Fluorescence (TRF) Assay, Surface plasmon resonance immunoassay (SPRIA), Western blotting (immunoblotting), and the like, are well known in the art, any of which may be used to detect antibodies in the serum of an individual to determine if ADAs have been generated. ADA titer may be assessed in an individual's serum following administration of the hypoimmunogenic biotherapeutic, where the level of ADA detected in serum collected 24 hours or more, e.g. 48 hours or 78 hours or more, in some instances 1, 2, 3, or 4 weeks or more, e.g. 6 weeks or 8 weeks after administration of the hypoimmunogenic therapeutic will be lower than the level of ADAs detected in serum from a control individual treated with the same dosing regimen for the same duration with a corresponding unengineered biotherapeutic.

As another example, immunosuppression may be observed in vitro as a reduction in leukocyte response upon exposure to the engineered biotherapeutic relative to a corresponding unengineered biotherapeutic. For example, greater activation of downstream signaling pathways (e.g. Erk phosphorylation, NFAT nuclear translocation) will be observed in a B cell comprising a CD22 Siglec that is exposed to an unengineered biotherapeutic as compared to a B cell comprising a CD22 Siglec that is exposed to a biotherapeutic that has been modified to comprise more CD22 ligand. As yet another example, a CD22 Siglec—and likewise, a cell expressing a CD22 Siglec—will have higher binding affinity for a biotherapeutic that has been engineered to comprise more CD22 ligand than an unengineered biotherapeutic.

In some embodiments, the ADAs to the hypoimmunogenic biotherapeutic are lower in a treated individual's serum after administering the subject biotherapeutic for one day or more, for example, one month or more, 6 months or more, 9 months or more, or 1 year or more, relative to the level of ADAs detected in serum from a control individual treated with the same dosing regimen and for the same duration with a corresponding unengineered biotherapeutic. In certain embodiments, the ADAs to the hypoimmunogenic biotherapeutic are undetectable in an individual's serum after administering the subject biotherapeutic for one month or more, e.g. 6 months or more, 9 months or more, or 1 year or more, whereas ADAs can be detected in serum from a control individual treated with the same dosing regimen and for the same duration with a corresponding unengineered biotherapeutic. Such administration may be daily, weekly, biweekly, monthly, quarterly, semi-annually, annually, bi-annually, once every 3 years, once every 4 years, once every 5 years, or once every 10 years.

In some embodiments, an engineered hypoimmunogenic biotherapeutic of the present disclosure may be further engineered to comprise elevated amounts of a ligand for an Asialoglycoprotein receptor (ASGPR). Asialoglycoprotein receptors are lectins which bind asialoglycoprotein and glycoproteins from which a sialic acid has been removed to expose galactose residues. Ligands for ASGPR, such as a galactosylating moieties (galactose, galactosamine, N-acetylgalactosamine (GalNAc)), glucosylating moieties (glucose, glucosamine, N-acetylglucosamine (GlcNAc)) and glycomimetics thereof, when covalently bound with an antigen that would normally elicit a T cell response, have been shown to induce immune tolerance to the antigen instead; see, e.g. US20170007708A1, the full disclosure of which is incorporated herein in its entirety. In certain embodiments, the ASGPR ligand is naturally occurring galactosylating moiety such as galactose, galactosamine, or GalNAc. In other embodiments, the ASGPR ligand is glucosylating moiety such as glucose, glucosamine, or GlcNAc. In other embodiments, the ASGPR ligand is a synthetic ligand, e.g. a glycomimetic as disclosed in, for example, Mamidyala, S K et al. (2012) J. Am. Chem. Soc. 2012, 134, 4, 1978-1981.

Methods for covalently associating ASGPR ligands to a biotherapeutic are well known in the art (see, e.g. US20170007708A1, the full disclosure of which is incorporated herein by reference), any of which may be used to engineer the biotherapeutics of the present disclosure. In some embodiments, the hypoimmunogenic biotherapeutic comprises at least 2-fold more ASGPR ligand than a corresponding unengineered biotherapeutic that would induce a T cell response in the individual, for example, 3-fold more, 4-fold more, 5-fold more, 6-fold more, 7-fold more, 8-fold more, 9-fold more 10-fold more, 11-fold more, 12-fold more, 13-fold more, 14-fold more, 15-fold more, 16-fold more, 17-fold more, 18-fold more, 19-fold more, or even 20-fold more ASGPR ligand than the unengineered biotherapeutic. Any approach for measuring the content of glycan on a biotherapeutic composition, including, e.g., glycoprotein LC/MS, Glycan LC/MS, capillary gel electrophoresis glycan analysis, analytical ion exchange HPLC, etc. may be used to determine the amount of ASGPR ligand appended to a biotherapeutic.

The covalent association of an ASGPR ligand to the subject hypoimmunogenic biotherapeutic is expected to direct the subject hypoimmunogenic biotherapeutic to antigen presenting cells of the liver (particular binding to hepatocytes and specifically ASGPR). Specificity in binding to antigen-presenting cells in the liver can be confirmed by, for example, labeling the subject hypoimmunogenic biotherapeutic comprising ASGPR ligand with a marker (such as the fluorescent marker phycoerythrin (“PE”)). The subject biotherapeutic is administered to suitable experimental subjects. Controls, e.g., unconjugated PE or vehicle (saline) are administered to other group(s) of subjects. The subject biotherapeutic and controls are allowed to circulate for a period of 1 to 5 hours, after which the spleens and livers of the subjects are harvested and measured for fluorescence. The specific cells in which fluorescence is found can be subsequently identified. The subject ASGPR ligand-associated biotherapeutic, when tested in this manner, show higher levels of concentration in the antigen-presenting cells of the liver as compared with unconjugated PE or vehicle.

Effectiveness in immune modulation can be tested by measuring the proliferation of OT-1 CD8+ cells (transplanted into host mice) in response to the administration of the subject hypoimmunogenic biotherapeutic comprising ASGPR ligand as compared with administration of the hypoimmunogenic biotherapeutic alone or just vehicle. The ASGPR ligand-associated biotherapeutic, when tested in this manner, shows an increase of OT-1 cell proliferation as compared with biotherapeutic alone or vehicle, demonstrating increased CD8+ T-cell cross-priming. To distinguish T cells being expanded into a functional effector phenotype from those being expanded and deleted, the proliferating OT-1 CD8+ T cells can be phenotypically analyzed for molecular signatures of exhaustion [such as programmed death-1 (PD-1), FasL, and others], as well as Annexin-V binding as a hallmark of apoptosis and thus deletion. The OT-1 CD8+ T cells can also be assessed for their responsiveness to challenge with unengineered biotherapeutic plus adjuvant in order to demonstrate functional non-responsiveness, and thus immune tolerance, towards the biotherapeutic. To do so, the cells are analyzed for inflammatory signatures after administration of compositions of the disclosure into host mice followed by a protein challenge. Compositions of the disclosure when tested in this manner demonstrate very low (e.g., background) levels of inflammatory OT-1 CD8+ T cell responses towards the biotherapeutic in comparison to control groups, thus demonstrating immune tolerance.

Effectiveness of the subject ASGPR ligand-associated biotherapeutic at inducing tolerance in humans can be assessed by assessing inflammatory signatures associated with T cell responses. Typically, a biotherapeutic to which an ASGPR ligand has been covalent associated will elicit a T cell response that is 50% of the T cell response elicited by a corresponding biotherapeutic or less in an individual administered the biotherapeutic, for example, 40%, 30%, or 20% or less, in certain instances, 15%, 10%, 5% or less, preferably only 2%, 1% or less of that which is elicited by a corresponding unengineered biotherapeutic. The induction of tolerance by the ASGPR-associated hypoimmunogenic biotherapeutic can be readily assessed by quantifying anti-biotherapeutic antibody titer specific to the unengineered biotherapeutic administered several weeks following treatment(s) with an ASGPR ligand-associated biotherapeutic. Compositions of the disclosure when tested in this manner show low levels of antibody formation responsive to challenge with the biotherapeutic in groups pretreated with ASGPR ligand-associated biotherapeutics as compared to groups that are not pretreated.

As discussed above, the engineered hypoimmunogenic biotherapeutics of the present disclosure retain pharmacologic activity while comprising the one or more modifications disclosed herein that render the biotherapeutic capable of suppressing an immune response. By “retain[ing] pharmacologic activity”, it is meant that the biotherapeutic is no more than 5-fold less therapeutically active than the corresponding unengineered biotherapeutic would be when administered to a naïve individual (that is, an individual receiving the therapy for the first time), in some cases, no more than 3-fold less active, preferably no more than 2-fold less active, more preferably at least as therapeutically active as the corresponding unengineered biotherapeutic would be when administered to a naïve individual.

In some cases, when Y is a polypeptide, the polypeptide conjugation reactive terminus of the linker is in some cases a site that is capable of conjugation to the polypeptide through a cysteine thiol or lysine amine group on the polypeptide, and so can be a thiol-reactive group such as a maleimide or a dibromomaleimide, or as defined herein, or an amine-reactive group such as an active ester (e.g., perfluorophenyl ester or tetrafluorophenyl ester), or as defined herein. Stated in another manner, the connection between Y and L or X can be the produce of a reaction between cysteine, thiol or lysine amine group on the polypeptide and a thiol-reactive group such as a maleimide or dibromomaleimide on the L or X group, or an amine-reactive group on the L or X group.

In some embodiments, the X or L group is covalently bound to a terminal end of an amino acid residue or a glycan on the biotherapeutic Y that is not typically sialylated, i.e. the sialic acid residue is heterologous to the amino acid residue or glycan moiety. As used herein, the term “heterologous” refers to a component of a composition that is non-native to the composition, i.e. not typically found in nature in association with the rest of the entity to which it is being compared. For example, the sialic acids may be covalently bound to a glycan structure such as G0, G1, G2, G0F, G1F, or G2F. In some embodiments, the sialic acid is covalently bound to structure that is typically sialylated in a glycan such as G1S, G2S, G2S2, G1FS, G2FS, and G2FS2. In some embodiments, the sialic acid is covalently bound to a native glycan, N- or O-linked, present in the corresponding unengineered biotherapeutic lacking the sialic acid modification. In other such embodiments, the sialic acid is covalently bound to a novel N-linked glycan site. In other embodiments, the sialic acid is covalently bound directly to an amino acid of the biotherapeutic, e.g. a random lysine or cysteine, an engineered transglutaminase site, an engineered Catalent formylglycine aldehyde site using formylglycine-generating enzyme (FGE), N-terminus-selective conjugation to biotherapeutics containing an N-terminal 2-hydroxyethylamine (Serine) moiety (SeriMab technology), or a novel 0-linked glycan site. Any approach for determining the sites of sialylation or Siglec ligand conjugation on a biotherapeutic, including, e.g., proteolyzed product LC/MS (peptide mapping LC/MS), and LC/MS of larger product fragments (e.g., antibody Fc vs light chain, Fd′), may be used to determine the placement of Siglec ligand within the biotherapeutic.

In some embodiments, the X or L is covalently bound to the native element, i.e. glycan, of the biotherapeutic Y.

### Additional Aspects

As discussed above, in some aspects of the disclosure, an engineered hypoimmunogenic biotherapeutic is provided, wherein the engineered hypoimmunogenic biotherapeutic (referred to hereafter as the “hypoimmunogenic biotherapeutic”, “modified biotherapeutic” or simply “subject biotherapeutic”) is a biotherapeutic that has been engineered to comprise an altered Siglec ligand profile.

In some embodiments, the altered Siglec ligand profile will comprise an enrichment for sialic acid relative to the parental biotherapeutic. Put another way, the engineered hypoimmunogenic biotherapeutic is enriched for sialic acid, i.e. it is “hypersialylated”. For example, the engineered hypoimmunogenic biotherapeutic may comprise one or more sialic acid moieties, e.g. 1, 2, 3, 4 or more sialic acid moieties, in some cases 5, 6, 7, 8, 9, or 10 or more sialic acid moieties, in some such instances, 11, 12, 13, 14, 15, 16, 17, 18, 19, or 20 or more moieties, whereas the parental biotherapeutic comprises no sialic acid moieties As another example, the subject biotherapeutic may comprise two or more sialic acid moieties, e.g. 2, 3, 4 or more sialic acid moieties, in some cases 5, 6, 7, 8, 9, or 10 or more sialic acid moieties, in some such instances, 11, 12, 13, 14, 15, 16, 17, 18, 19, or 20 or more sialic acid moieties, whereas the parental biotherapeutic comprises only one sialic acid moiety. In some embodiments, the hypoimmunogenic biotherapeutic comprises 2-fold sialic acid or more than a corresponding unengineered biotherapeutic that would induce an immune reaction in the individual, for example, 3-fold more, 4-fold more, 5-fold more, 6-fold more, 7-fold more, 8-fold more, 9-fold more 10-fold more, 11-fold more, 12-fold more, 13-fold more, 14-fold more, 15-fold more, 16-fold more, 17-fold more, 18-fold more, 19-fold more, or even 20-fold more sialic acid than the unengineered biotherapeutic. In some embodiments, 50% or more of the glycan moieties of the engineered hypoimmunogenic biotherapeutic, e.g., 60%, 70%, 80%, 85%, 90%, 95%, 98% or 100% of the glycan moieties, comprise a sialic acid.

In some embodiments, 50% or more of the hypoimmunogenic biotherapeutic in a sample is hypersialylated, e.g., 60%, 70%, 80%, 85%, 90%, 95%, 98% or 100% of the hypoimmunogenic biotherapeutic in a sample is hypersialylated. For example, 50% or more of the hypoimmunogenic biotherapeutic in a sample can be hypersialylated to the same extent or greater, which as described above includes embodiments where the hypoimmunogenic biotherapeutic comprises more sialic acid than a corresponding unengineered biotherapeutic that would induce an immune reaction in the individual. In some embodiments, 60%, 70%, 80%, 85%, 90%, 95%, 98% or 100% of the hypoimmunogenic biotherapeutic in a sample is hypersialylated to the same extent or greater.

Any approach for measuring the sialylation, i.e. the sialic acid content, of a biotherapeutic composition, including, e.g., glycoprotein LC/MS, Glycan LC/MS, protein LC/MS, intact drug LC/MS, capillary gel electrophoresis glycan analysis, analytical ion exchange HPLC, analytical reverse phase HPLC, analytical hydrophobic interaction chromatography HPLC, analytical mixed mode chromatography HPLC, total sialic acid or Siglec ligand analysis by plate-based assay, UV/Vis absorbance spectroscopy, surface plasmon resonance-based Siglec ligand quantitation assay, biolayer interferometry-based Siglec ligand quantitation assay, etc. may be used to determine the amount of Siglec ligand appended to a biotherapeutic.

In some embodiments, the Siglec ligand comprises a Siglec binding fragment from a Siglec-specific antibody, e.g. the CDR, the Fab, the Fab′, the Fv, the nanobody, etc. from, e.g., a monoclonal antibody, an scFv, a minibody, a diabody, a triabody, a tetrabody, a darpin, a camelid nanobody, an affimer, a fynomer, a bispecific antibody, a trispecific antibody, or the like that is specific for a Siglec. In some embodiments, the Siglec ligand comprises a Siglec binding fragment from a Siglec specific chimeric antigen receptor (“CAR”). In some embodiments, the Siglec-specific antibody or Siglec-specific CAR is specific for Siglec-2. In some such embodiments, the Siglec-2 specific antibody is selected from the group consisting of epratuzumab, inotuzumab, suciraslimab, bectumomab, pinatuzumab, GTB-1550, hLL2, RFB4, JNJ-75348780, HB-22.7, m971, H10-2-4, and moxetumomab In some such embodiments the Siglec ligand comprises a Siglec binding fragment derived from an scFv polypeptide sequence designed from epratuzumab, or a peptide selected from the group consisting of PV1 (GYINPRNDYTEYNQ), PV2 (CGYRNPRNDYREYCNQ), and PV3 (RNDYTE), the chemical structures for which may be found in Table 2 (Kim, B. et al. Nanoscale 2020, 12, 11672-11683). In certain such embodiments, the Siglec binding fragment consists essentially of an scFv polypeptide sequence designed from epratuzumab or a peptide selected from the group consisting of PV1 (GYINPRNDYTEYNQ), PV2 (CGYRNPRNDYREYCNQ), and PV3 (RNDYTE).

TABLE 2
Chemical structures for PV1, PV2, and PV3.
PV1

| | |
|---|---|
| Chemical Structure | 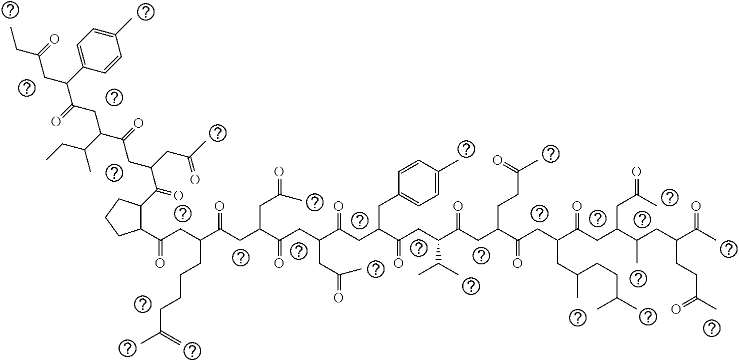 [SMILES: C=C(C)CCCCC(CC(=O)C1CCCC1C(=O)C(CC(C)=O)CC(=O)C(CC(=O)C(CC(=O)CC)c1ccc(C)cc1)C(C)CC)C(=O)CC(CC(C)=O)C(=O)CC(CC(C)=O)C(=O)CC(Cc1ccc(C)cc1)C(=O)C[C@H](C(=O)CC(CCC(C)=O)C(=O)CC(CC(C)CCC(C)C)C(=O)CC(CC(C)=O)C(C)CC(CCC(C)=O)C(C)=O)C(C)C] |

PV2

| | |
|---|---|
| Chemical Structure | 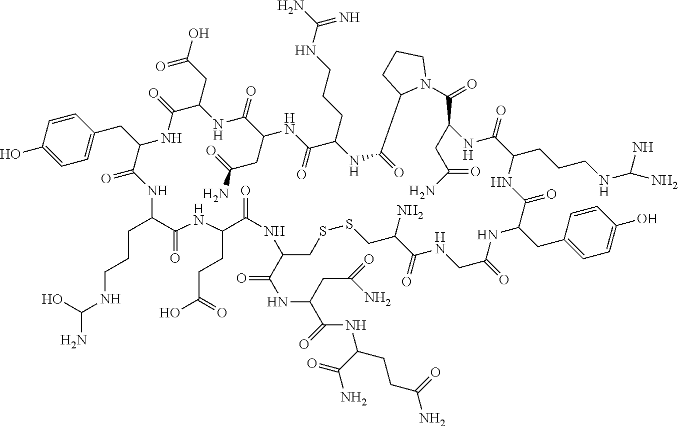 |

PV3

| | |
|---|---|
| Chemical Structure | 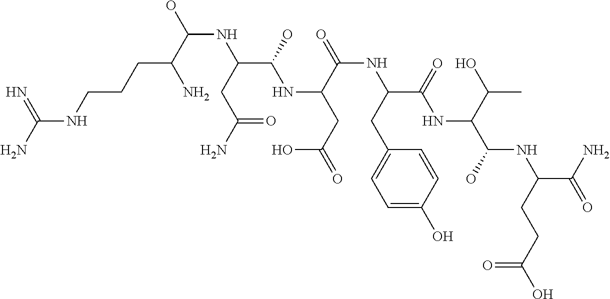 [SMILES: CC(O)C(NC(=O)C(Cc1ccc(O)cc1)NC(=O)C(CC(=O)O)N[C@@H](O)C(CC(N)=O)NC(O)C(N)CCCNC(=N)N)[C@@H](O)NC(CCC(=O)O)C(N)=O] |

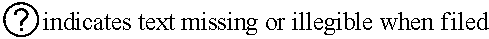

In some such embodiments, the Siglec ligand is a synthetic derived from monoclonal antibody polypeptides that include HB-22.5, 22.7, 22.23, 22.33, 22.13, and HB22.196, as described in Pearson, et al. (International Journal of Peptide Research and Therapeutics, 14, 3, 237-246 (2008)). One such peptide is “Peptide 5”, derived from the CDR2 region of monoclonal antibody HB22-7, with amino acid sequence CLGIIWGDGRTDYNSALKSRC and a disulfide bond between the N- and C-terminal cysteines.

In some embodiments, the Siglec ligand comprises a Siglec binding fragment from a Siglec-specific aptamer. Nonlimiting examples of Siglec-specific aptamers that comprise a Siglec binding fragment that finds use in the subject biotherapeutics include TD-05, TD-05.1, and TD-05.17.

In some such embodiments, the Siglec ligand comprises a synthetic, non-antibody-derived Siglec binding peptide, where the peptide binds with measurable affinity and high specificity to CD22. For example, peptides may be those described in WO2014044793, e.g., “Peptide 26”, otherwise known as “G635BVI07IM1TK” with amino acid sequence

RALLSIFGSLDHRHHHRTCNTTHYRVTTMSHPQFEKKKKLRMKMSHPQLI
NTTHYRGGPTMGGSPSRQV”.

Accordingly, in some embodiments, the subject engineered hypoimmunogenic biotherapeutic comprises a biotherapeutic conjugated to one or more naturally occurring Siglec ligands, i.e. a moiety comprising a naturally occurring sialic acid and a naturally occurring glycan, wherein the sialic acid and glycan are typically found in nature in association with one another to form a Siglec ligand. In other embodiments, the subject engineered hypoimmunogenic biotherapeutic comprises a biotherapeutic conjugated to one or more non-naturally occurring Siglec ligands, e.g. a moiety comprising a naturally occurring sialic acid and a linker, a moiety comprising a non-naturally occurring sialic acid and a glycan found in nature as part of a Siglec ligand, a moiety comprising a non-naturally occurring sialic acid and a linker, a moiety comprising a peptide having an affinity for a Siglec, and the like. For example, in some embodiments of the engineered hypoimmunogenic biotherapeutic, the Siglec ligand is a non-naturally occurring Siglec ligand. In some embodiments, the non-naturally occurring Siglec ligand comprises a naturally occurring sialic acid and a non-naturally occurring linker. In some embodiments, the non-naturally occurring Siglec ligand consists essentially of a naturally occurring sialic acid and a non-naturally occurring linker. In some embodiments, the non-naturally occurring Siglec ligand comprises a non-naturally occurring sialic acid. In some embodiments, the non-naturally occurring Siglec ligand comprises a non-naturally occurring linker. In some embodiments, the non-naturally occurring Siglec ligand consists essentially of a non-naturally occurring sialic acid and a non-naturally occurring linker.

### Methods of Manufacture

As discussed above, the hypoimmunogenic biotherapeutics are biotherapeutics which have been modified to comprise heterologous Siglec ligands (be they heterologous to the biotherapeutic or heterologous to the amino acid to which they are appended) and/or elevated amounts of Siglec ligand(s) that naturally occur on said biotherapeutics. Typically, the modification is not simply by associating the Siglec ligand with the biotherapeutic via a formulation, e.g. a liposomal formulation. Rather, the modification is a covalent binding of Siglec ligand to the biotherapeutic.

Methods of covalently binding sialic acids to biotherapeutic are well appreciated in the art, any of which may be deployed to modify a biotherapeutic of choice to become an engineered hypoimmunogenic biotherapeutic of the present disclosure. For example, the modification may be performed by engineered biosynthesis. By “biosynthesis”, it is meant a synthesis process that is mediated by cells. For example, in the Golgi apparatus, a subset of the 20 known sialyltransferases attach sialic acids to underlying monosaccharides such as galactose via three different types of linkage (α2,3, α2,6, and α2,8). By engineered biosynthesis, it is meant a synthesis process that is mediated by cells that have been engineered to perform the process, in some instances de novo, in other instances, in a modified way. Thus, for example, a producer cell line may be genetically engineering to express one or more sialyl transferases, e.g. sialyltransferase (EC 2.4.99), beta-galactosamide alpha-2,6-sialyltransferase (EC 2.4.99.1), alpha-N-acetylgalactosaminide alpha-2,6-sialyltransferase (EC 2.4.99.3), beta-galactoside alpha-2,3-sialyltransferase (EC 2.4.99.4), N-acetyllactosaminide alpha-2,3-sialyltransferase (EC 2.4.99.6), alpha-N-acetyl-neuraminide alpha-2,8-sialyltransferase (EC 2.4.99.8); lactosylceramide alpha-2,3-sialyltransferase (EC 2.4.99.9), or other enzymes in an enzymatic pathway, e.g. CMP-Neu5Ac hydroxylase, sialate-4-O-acetyl transferase, sialate-4-O-acetylesterase, sialate-7(9)-O-acetyltransferase, sialate-8-O-methyl transferase, sialate-9-)-acetyltransferase, etc. that drives the covalent binding of a specific sialic acid to the biotherapeutic or that targets specific novel amino acid residues for covalent modification with sialic acid. As another example, a producer cell line could be fed a precursor substrate that will be incorporated by the producer line into the manufactured biotherapeutic as a specific Siglec ligand. Any producer cell that finds use in the expression of proteins for use as therapeutic biotherapeutics may be used in this process, for example a mammalian cell (CHO, HEK, etc.), an insect cell (SF9, etc.), a bacterium, a protozoan (Leishmania, etc.). as disclosed in, e.g. WO2017093291, WO2019002512, WO2019234021, the full disclosures of which are incorporated herein in their entirety by reference.

As another example, the modification may be performed by chemical conjugation. By “chemical conjugation”, it is meant a process that occurs exogenous to a cell. Thus, for example, the Siglec ligand might be enzymatically or chemically linked to the biotherapeutic after biosynthesis from producer cell line. Nonlimiting examples of such in vitro processes are disclosed in U.S. Pat. Nos. 7,220,555, 6,376,475B, and 5,409,817, the full disclosures of which are incorporated herein by reference. In some such embodiments, a linker may be deployed to covalently link the sialic acid to the biotherapeutic. Many examples of linkers exist in the art, any of which may be used to chemically conjugate sialic acid(s) to the biotherapeutic to arrive at hypoimmunogenic biotherapeutics of the present disclosure.

As a third example, specifically directed to embodiments in which the Siglec ligand is a peptide or polypeptide sequence, e.g. an scFv or peptide derived from epratuzumab, e.g. PV1, PV2 or PV3, the modification may be performed by genetic engineering of the biotherapeutic to comprise the peptide/polypeptide sequence within the biotherapeutic. For example, the polynucleotide used to produce the biotherapeutic may be modified by standard molecular biology cloning techniques to include a polynucleotide sequence encoding the peptide/polypeptide in the same translational reading frame (“In frame”), such that upon transcription and translation of the biotherapeutic in a producing cell, the biotherapeutic will comprise the peptide/polypeptide sequence covalently associated with amino acids that make up the biotherapeutic, resulting in a biotherapeutic that is hypoimmunogenic. Preferably, the peptide/polypeptide sequence will be genetically engineered into a domain of the biotherapeutic that is not responsible for the therapeutic effect of the biotherapeutic, e.g. the enzymatic domain of an enzyme, the Fab or more specifically CDR domains of an antibody, etc. In the instance of modifying a viral particle, the peptide/polypeptide sequence will preferably be genetically engineered into a capsid or envelop protein so as to be exposed to the exterior of the viral particle, e.g. into an exposed loop of a viral capsid protein, a surface-exposed tegument protein, etc. Such structural features are well understood by one of ordinary skill in the art of viral therapies.

### Methods of Use

In some aspects of the invention, methods are provided for the treatment of individuals in need of a medical intervention. The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

The hypoimmunogenic compositions of the present disclosure find particular use in the treatment of diseases that require repeat or chronic administration of the therapeutic to be effective. There are many instances of such conditions, of which a few nonlimiting examples are provided below and elsewhere. It is expected that the ordinarily skilled artisan will be able to extrapolate from these examples to other indications and biotherapeutics as known in the art.

For example, the individual may be suffering from a chronic autoimmune or inflammatory disease, e.g. rheumatoid arthritis, psoriatic arthritis, ankylosing spondylitis, Crohn's disease, ulcerative colitis, psoriasis, hidradenitis suppurativa, uveitis, and juvenile idiopathic arthritis. In such instances, the method may comprise administering to the individual a hypoimmunogenic TNFα-specific antibody, e.g. a hypoimmunogenic adalimumab engineered from adalimumab, or a hypoimmunogenic infliximab engineered from infliximab, in an amount effective to treat the chronic immune disease.

As another example, the individual may be suffering from a leukemia, e.g. ALL. In such instances, the method may comprise administering to the individual an engineered hypoimmunogenic asparaginase from Erwinia chrysanthemi in an amount effective to treat the leukemia.

As another example, the individual may be suffering from a colorectal cancer, a non-small cell lung cancer, or a head and neck cancer. In such instances, the method may comprise administering to the individual an engineered hypoimmunogenic cetuximab in an amount effective to treat the colorectal cancer, non-small cell lung cancer, or head and neck cancer.

As another example, the individual may be suffering from multiple sclerosis. In such instances, the method may comprise administering to the individual an engineered hypoimmunogenic natalizumab, an engineered hypoimmunogenic IFNβ-1b, or an engineered hypoimmunogenic IFNβ-1a in an amount effective to treat the multiple sclerosis.

As another example, the individual may be the recipient of an organ transplant and in need of an immunosuppressive agent that protects the transplanted tissue from rejection by the individual's immune system. In such instances, the method may comprise administering to the individual an engineered hypoimmunogenic IdeS in an amount effective to prevent an antibody response to the transplanted tissue. In some embodiments, the transplanted organ is an allogeneic graft. In some embodiments, the transplanted organ is a xenogeneic graft. In some embodiments, the organ is selected from kidney, heart, lung, liver, pancreas, trachea, vascular tissue, skin, bone, cartilage, adrenal tissue, fetal thymus, and cornea.

As another example, the individual may be suffering from Type 2 Diabetes. In such instances, the method would comprise administering to the individual an engineered hypoimmunogenic exenatide or engineered hypoimmunogenic albiglutide in an amount effective to treat the diabetes.

As another example, the individual may be suffering from a complement-mediated disease. In such instances, the method would comprise administering to the individual an engineered hypoimmunogenic complement degrading protease, e.g. from a pathogen such as a bacterial pathogen or fungal pathogen (e.g. Pseudomonas Elastase (PaE), Pseudomonas Alkaline protease (PaAP), Streptococcal pyrogenic Exotoxin B (SpeB), a gingipain from Porphyromonas gingivalis, Aspergillus Alkaline protease 1 (Alp1), C. albicans Secreted aspartyl proteinases 1 (Sap1), 2 (Sap2), and 3 (Sap3), in an amount effective to degrade complement and treat the disease.

As another example, the individual may be suffering from an enzyme deficiency. In such instances, the method would comprise administering to the individual an engineered hypoimmunogenic enzyme in an amount effective to treat the deficiency. Nonlimiting examples of such enzyme deficiencies would include PKU, wherein a hypoimmunogenic phenylalanine ammonia-lyase would be administered; Fabry disease, wherein a hypoimmunogenic alpha-galactosidase A would be administered; Pompe disease, wherein a hypoimmunogenic acid α-glucosidase (GAA) would be administered; Gaucher disease, wherein a hypoimmunogenic glucocerebrosidase (GCase) would be administered; Aspartylglucosaminuria, wherein a hypoimmunogenic aspartylglucosaminidase (AGA) would be administered; Hypophosphatasia (HPP), wherein a hypoimmunogenic asfotase would be administered; MPS I, wherein a hypoimmunogenic alpha-L-iduronidase would be administered; MPS II, wherein a hypoimmunogenic iduronate sulfatase would be administered; MPS Ilila, wherein a hypoimmunogenic sulfaminase would be administered; MPS IIIB, wherein a hypoimmunogenic α-N-acetylglucosaminidase (NAGLU) would be administered; MPS IIIC, wherein a hypoimmunogenic heparin acetyle CoA: α-glucosaminide N-acetyltransferase (HGSNAT) would be administered; MPS IIID, wherein a hypoimmunogenic N-acetylglucosamine 6-sulfatase (GNS) would be administered; MPS IIIE, wherein a hypoimmunogenic N-glucosamine 3-O-sulfatase (arylsulfatase G or ARSG) would be administered; MPS IVA, wherein a hypoimmunogenic N-acetylgalactosamine 6-sulfatase would be administered; MPS IVB, wherein a hypoimmunogenic beta-galactosidase would be administered; MPS VI, wherein a hypoimmunogenic N-acetylgalactosamine 4-sulfatase would be administered; MPS VI, wherein a hypoimmunogenic beta-glucuronidase would be administered; Hemophilia A, wherein a hypoimmunogenic Factor VIII would be administered; Hemophilia B, wherein a hypoimmunogenic Factor IX would be administered; the CLN1 form of Batten Disease, wherein a hypoimmunogenic palmitoyl protein thioesterase (PPT1) would be administered; the CLN2 form of Batten Disease, wherein a hypoimmunogenic Tripeptidyl peptidase (TPP1) would be administered; arginase-1 deficiency, wherein a hypoimmunogenic arginase-1 or pegzilarginase would be administered; and cystathionine beta synthase (CBS) deficiency, also known as Classical Homocystinuria, wherein a hypoimmunogenic cystathionine beta synthase or Aeglea product AGLE-177 is administered.

As another example, the individual may be suffering from disease that would benefit from a gene therapy, e.g. a genetic disease, or a complex disease (i.e. not restricted to being associated with a specific genetic etiology) in which chronic expression of a therapeutic RNA or protein would treat the condition. In such instances, the method would comprise administering to the individual an engineered hypoimmunogenic viral particle comprising a polynucleotide sequence (a “transgene”) encoding the therapeutic gene product of interest, in an amount effective to treat the disease. Nonlimiting examples of suitable transgenes/gene products that one might deliver via the subject hypoimmunogenic viral particle include those associated with muscular dystrophy, cystic fibrosis, familial hypercholesterolemia, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), among others. See, e.g., www.orpha.net/consor/cgibin/Disease_Search_List.php; rarediseases.info.nih.gov/diseases.

Other useful therapeutic gene products that could be encoded by the transgene also include hormones and growth and differentiation factors including, without limitation, insulin, glucagon, glucagon-like peptide 1 (GLP-1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor a (TGFa), platelet-derived growth factor (PDGF), insulin growth factors I and II (IGF-I and IGF-II), any one of the transforming growth factor b superfamily, including TGF b, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgenes include those that encode proteins that regulate the immune system including, without limitation, cytokines and lymphokines such as thrombopoietin (TPO), interleukins (IL) IL-1 through IL-25 (including, IL-2, IL-4, IL-12, and IL-18), monocyte chemoattractant protein, leukemia inhibitory factor, granulocyte-macrophage colony stimulating factor, Fas ligand, tumor necrosis factors a and b, interferons a, b, and g, stem cell factor, Hk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful transgenes include those that encode gene products for any one of the receptors for the hormones, growth factors, cytokines, lymphokines, regulatory proteins and immune system proteins.

Still other useful transgenes include those encoding receptors for cholesterol regulation, including the low density lipoprotein (LDL) receptor, high density lipoprotein (HDL) receptor, the very low density lipoprotein (VLDL) receptor, and the scavenger receptor. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-e-phosphatase, porphobilinogen deaminase, Factor VIII, Factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin sequence or functional fragment thereof. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encodes b-glucuronidase (GUSB)). In another example, the gene product is ubiquitin protein ligase E3A (UBE3A). Still useful gene products include UDP Glucuronosyltransferase Family 1 Member A1 (UGT1A1).

In some embodiment, the gene product is not Factor VIII.

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable transgenes include those which encode therapeutics that may be useful for treating individuals suffering from autoimmune diseases and disorders by conferring a broad based protective immune response against targets that are associated with autoimmunity including cell receptors and cells which produce self-directed antibodies. T cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single mini gene separated by 42 nucleic acids coding for 14 amino acids of the B domain [U.S. Pat. No. 6,200,560]

Further illustrative genes which may be delivered via the hypoimmunogenic viral particle include, without limitation, glucose-6-phosphatase, associated with glycogen storage disease or deficiency type 1A (GSD1), phosphoenolpyruvate-carboxy kinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxy acid Oxidase 1 (GO/HAOI) and AGXT, branched chain alpha-ketoacid dehydrogenase, including BCKDH, BCKDH-E2, BAKDH-Ela, and BAKDH-Elb, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type CI); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; a-fucosidase associated with fucosidosis; a-mannosidase associated with alpha-mannosidosis; porphobilinogen deaminase, associated with acute intermittent Porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

### Pharmaceutical Compositions

In methods of treating an individual with the subject hypoimmunogenic biotherapeutic, the patient will typically be administered a pharmaceutical composition comprising the subject hypoimmunogenic biotherapeutic. By a pharmaceutical composition, it is meant an engineered hypoimmunogenic biotherapeutic of the present disclosure that has been formulated in a pharmaceutically acceptable carrier. As used herein, a “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The pharmaceutical compositions of the disclosure are administered at a therapeutically effective dosage, e.g., a dosage sufficient to provide treatment for the disease states previously described. Administration of the compounds of the disclosure or the pharmaceutically acceptable salts thereof can be via any of the accepted modes of administration for agents that serve similar utilities. While human dosage levels have yet to be optimized for the compounds of the disclosure, these can be readily extrapolated from doses administered to a relevant animal model, e.g. mice that results in treatment of the disease or disorder in that animal model. Generally, an individual human dose is from about 0.01 to 2.0 mg/kg of body weight, preferably about 0.1 to 1.5 mg/kg of body weight, and most preferably about 0.3 to 1.0 mg/kg of body weight. Treatment can be administered for a single day or a period of days, and can be repeated at intervals of several days, one or several weeks, or one or several months. Administration can be as a single dose (e.g., as a bolus) or as an initial bolus followed by continuous infusion of the remaining portion of a complete dose over time, e.g., 1 to 7 days. The amount of active compound administered will, of course, be dependent on any or all of the following: the subject and disease state being treated, the severity of the affliction, the manner and schedule of administration and the judgment of the prescribing physician. It will also be appreciated that amounts administered will depend upon the molecular weight of the biotherapeutic, the amount of Siglec ligand covalently bound, and the size of the linker.

While all typical routes of administration are contemplated (e.g. oral, topical, transdermal, injection (intramuscular, intravenous, or intra-arterial)), it is presently preferred to provide liquid dosage forms suitable for injection. Generally, depending on the intended mode of administration, the pharmaceutically acceptable composition will contain about 0.1% to 95%, preferably about 0.5% to 50%, by weight of the subject hypoimmunogenic biotherapeutic of the disclosure, the remainder being suitable pharmaceutical excipients, carriers, etc. Dosage forms or compositions containing active ingredient in the range of 0.005% to 95% with the balance made up from non-toxic carrier can be prepared.

The subject pharmaceutical compositions can be administered either alone or in combination with other pharmaceutical agents. These compositions can include other medicinal agents, pharmaceutical agents, carriers, and the like, including, but not limited to other active agents that can act as immune-modulating agents and more specifically can have inhibitory effects on B-cells, including anti-folates, immune suppressants, cyostatics, mitotic inhibitors, and anti-metabolites, or combinations thereof.

Liquid pharmaceutically administrable compositions can, for example, be prepared by dissolving, dispersing, etc. an active composition of the disclosure (e.g., a lyophilized powder) and optional pharmaceutical adjuvants in a carrier, such as, for example, water (water for injection), saline, aqueous dextrose, glycerol, glycols, ethanol or the like (excluding galactoses), to thereby form a solution or suspension. If desired, the pharmaceutical composition to be administered can also contain minor amounts of nontoxic auxiliary substances such as wetting agents, emulsifying agents, stabilizing agents, solubilizing agents, pH buffering agents and the like, for example, sodium acetate, sodium citrate, cyclodextrine derivatives, sorbitan monolaurate, triethanolamine acetate and triethanolamine oleate, etc., osmolytes, amino acids, sugars and carbohydrates, proteins and polymers, salts, surfactants, chelators and antioxidants, preservatives, and specific ligands. Actual methods of preparing such dosage forms are known, or will be apparent, to those skilled in this art; for example, see Remington: The Science and Practice of Pharmacy, Pharmaceutical Press, 22nd Edition, 2012. The composition or formulation to be administered will, in any event, contain a quantity of the active compound in an amount effective to treat the symptoms of the subject being treated.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### General Synthetic Procedures

All synthetic chemistry was performed in standard laboratory glassware unless indicated otherwise in the examples. Commercial reagents were used as received. Microwave reactions were performed in an Anton Paar Monowave 400 using the instrument software to control heating time and pressure. Analytical LC/MS was performed either on a Waters Acquity UPLC Instrument with PDA and Single Quadrupole Detector (with alternating positive and negative ion scans) using Masslynx Software or a Shimadzu LCMS-2020 using LabSolutions software. Retention times were determined from the extracted 214 and/or 254 nm UV chromatogram. Prep HPLC was performed either on a Waters Autopurification System consisting of Fraction module 2767, Pump 2545 and 2998 PDA detector using Masslynx software/Agilent 1260 Infinity Autopurification system with DAD detector or on a Gilson system using a 215 liquid handler, 333 and 334 pumps, UV/VIS-155 detector, and Trilution Ic software. ¹H NMR was performed either on a Bruker Avance 400 MHz or a Bruker Fourier 300 MHz using Topspin software. Analytical thin layer chromatography was performed on silica (Sigma Aldrich TLC Silica gel 60 F₂₅₄ aluminum or glass TLC plate, silica gel coated with flourescent indicator F254) and is visualized under UV light. Silica gel chromatography was performed manually, or with Teledyne ISCO CombiFlash NextGen 300+ automated chromatography for gradient elution.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/I, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Example 1: Concept for Class of CD22-Engaging Biotherapeutics that are Suppressed for Anti-Drug Antibody Responses

The B cell and its clonotypic B cell receptor sit at the heart of antibody-based immune responses to foreign agents. Anti-drug antibodies are a ubiquitous challenge to drug exposure for many biotherapeutic drug classes, including monoclonal antibodies, bispecific and multispecific antibodies, enzyme replacement therapy drugs, recombinant microbial enzymes, protein-Fc fusion proteins, intracellular delivery constructs, and gene therapy vectors.

A novel class of biomolecules and biotherapeutics with suppressed or ablated humoral immunogenicity has been designed, based on the principal that B cell clones with the potential to differentiate into anti-drug antibody-secreting plasma cells can be inhibited through Siglec inhibitory receptor recruitment to clonotypic anti-drug B cell receptors. The Siglecs are a class of sialic acid-binding lectin proteins, expressed on most or all types of hematopoietic cells.

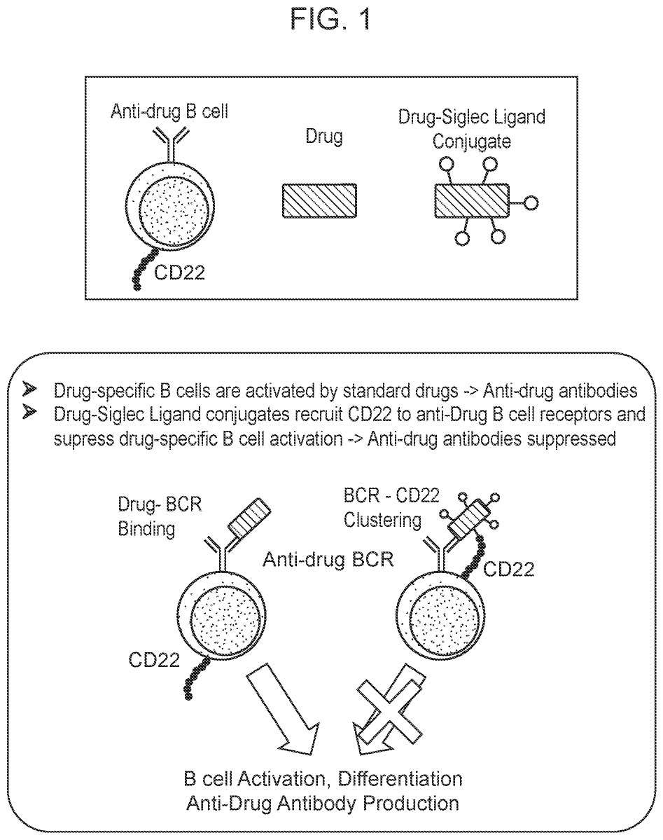

FIG. 1 depicts one aspect of the model for CD22-engaging biotherapeutics with suppressed anti-drug antibody responses. B cell receptor-Siglec Ligand co-engagers (including Drug-Siglec Ligand conjugates) suppress or silence drug-specific B cell activation by virtue of the physical recruitment of the inhibitory CD22 receptor to the B cell receptor complex.

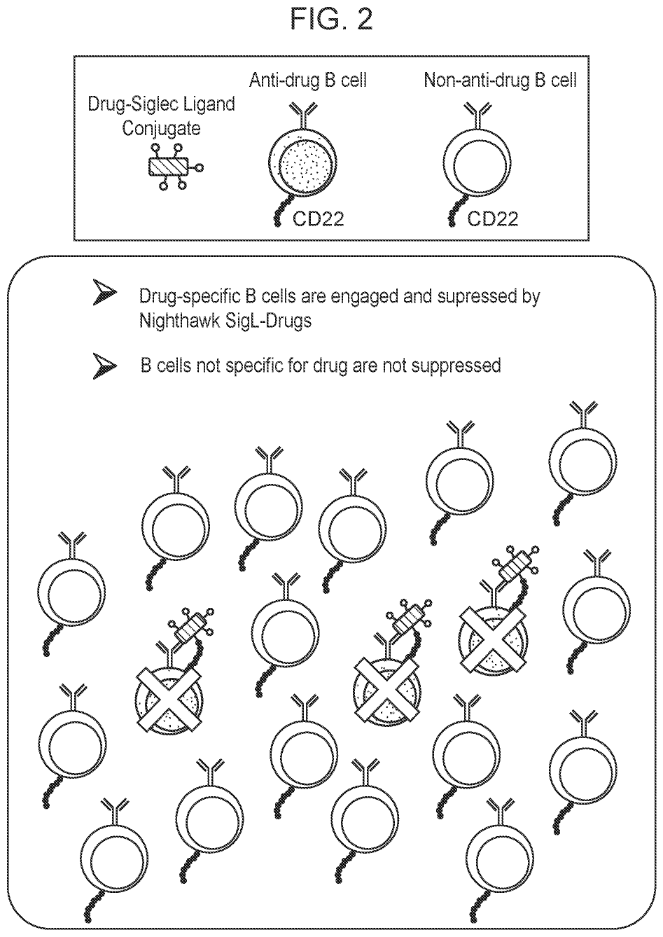

FIG. 2 depicts another aspect of the model for CD22-engaging biotherapeutics with suppressed anti-drug antibody responses: B cell receptor-Siglec Ligand co-engagers (including Drug-Siglec Ligand conjugates) suppress, silence, or delete only drug-specific B cells while leaving intact those B cell clones not specific for drug.

### Example 2: Different Formats for Siglec-2/CD22-Engaging, Hypo- or Non-Immunogenic Biotherapeutics

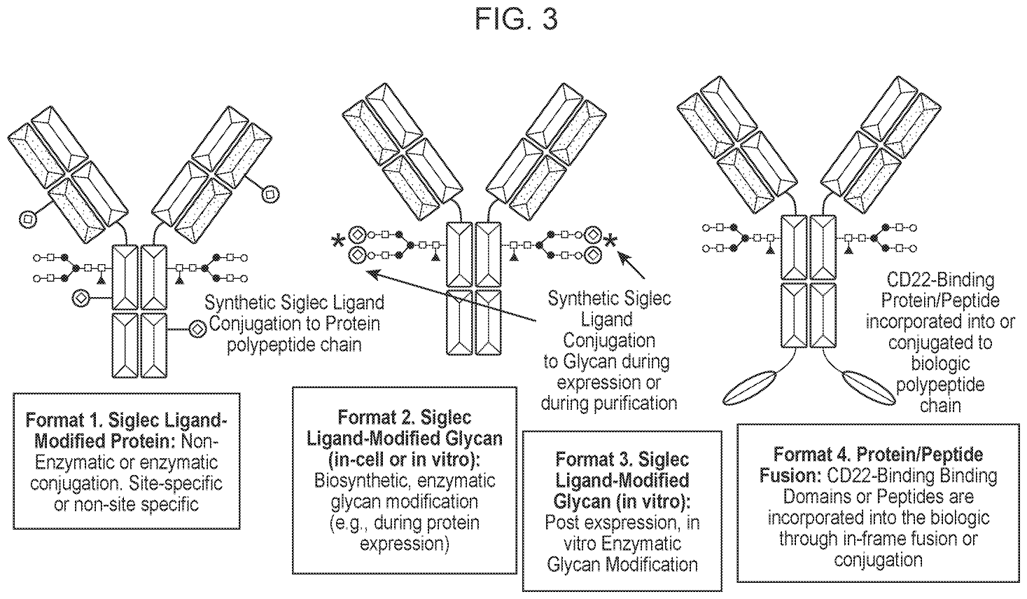

Four formats of Siglec-2/CD22-engaging, hypo- or non-immunogenic biotherapeutics may be engineered. FIG. 3 illustrates a representative structure of each format. An antibody is illustrated to exemplify such a biotherapeutic, but other biologic modalities (e.g., bispecific and multispecific antibodies, enzyme replacement therapy drugs, recombinant microbial enzymes, protein-Fc fusion proteins, intracellular delivery constructs, gene therapy vectors) may be similarly engineered using the four presented formats. Formats 1, 2, and 3 use chemically incorporated, synthetic, small-molecule Siglec ligands to impart Siglec-2 binding activity to the given drug. Such synthetic Siglec-2 Ligand structures are described in Example 3.

“Format 1” is a biotherapeutic covalently modified on its polypeptide chains with one or more conjugatable Siglec ligand-linker structures. Conjugation of the Siglec-2 ligand-linker structure can be achieved through site-specific or non-site-specific methodologies.

“Format 2” is a biotherapeutic modified on a natural or engineered glycan with Siglec ligand structures, where Siglec-2 ligand incorporation occurs biosynthetically during drug expression in cells. Such an approach would include approaches where a Siglec-2 ligand-based substrate would be fed to cells during drug expression to enable biosynthetic incorporation in drug glycans. Incorporation of Siglec ligand into glycan could also be achieved through treatment with Siglec-2 ligand-based enzyme substrate in an in vitro protein translation system.

“Format 3” is a biotherapeutic covalently modified on a natural or engineered glycan with Siglec ligand structures. Glycan modification with terminal Siglec-2 ligand structures is achieved through chemical and/or chemoenzymatic conjugation after purification of the biologic.

“Format 4” for engineering of a hypo- or non-immunogenic biologic relies on the incorporation of a protein- or peptide-based CD22 binder i into the polypeptide chain of the biotherapeutic. Examples of such CD22 binders would include: 1) immunoglobulin-based binders, such as Fab domains, single-chain Fv (scFv) fragments, diabodies, and single-domain antibody fragments (camelid V_{H}H or shark V_{NAR}); 2) non-immunoglobulin-based binding domains, such as affibodies, fynomers, monobodies, DARPins, Knottins, Variable Lymphocyte Receptors (VLRs), and affimers; 3) CD22-binding peptides, such as peptide aptamers; and 4) oligonucleotide-based Siglec binders, such as oligonucleotide aptamers.

All four of the illustrated formats would enable CD22 recruitment to anti-drug, clonotypic B cell receptors on drug-specific B cells, with consequent suppression of B cell activation, proliferation, and differentiation, ultimately blocking anti-drug antibody production.

### Example 3

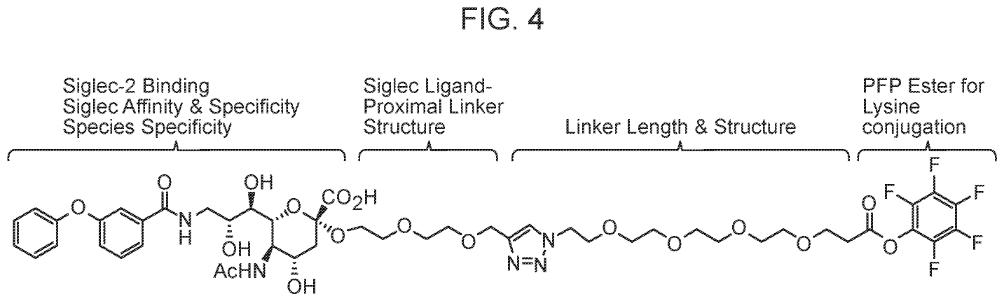

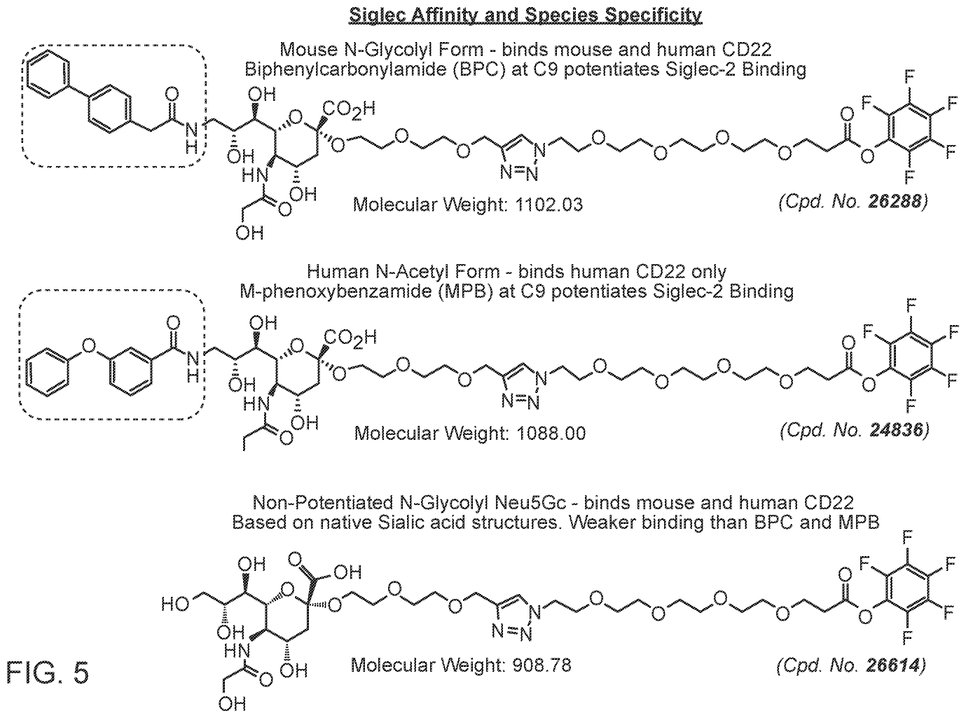

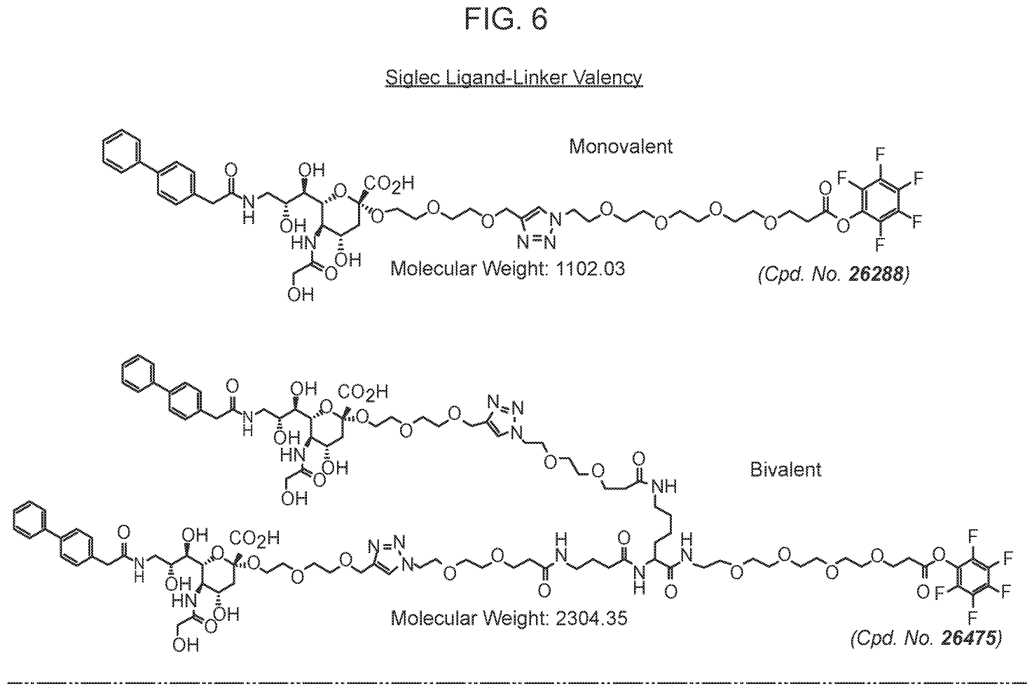

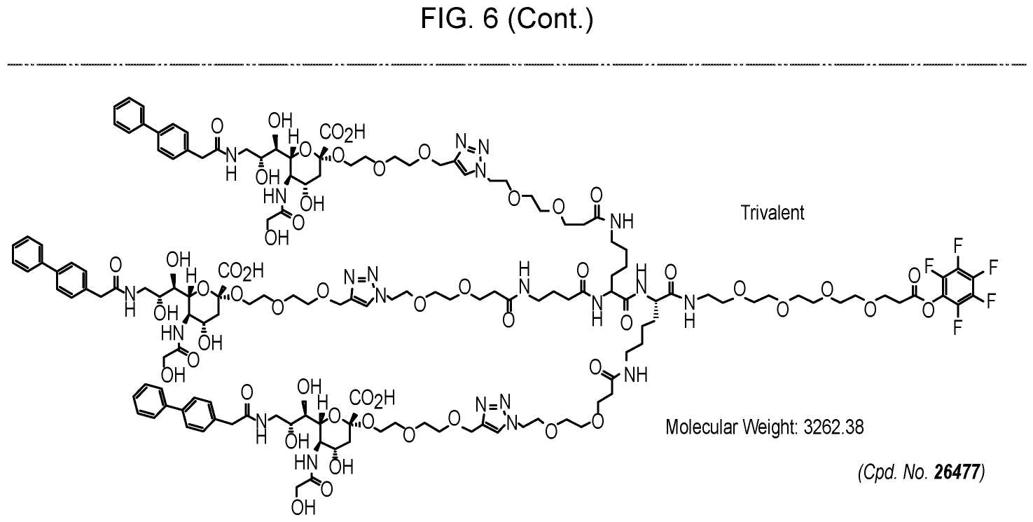

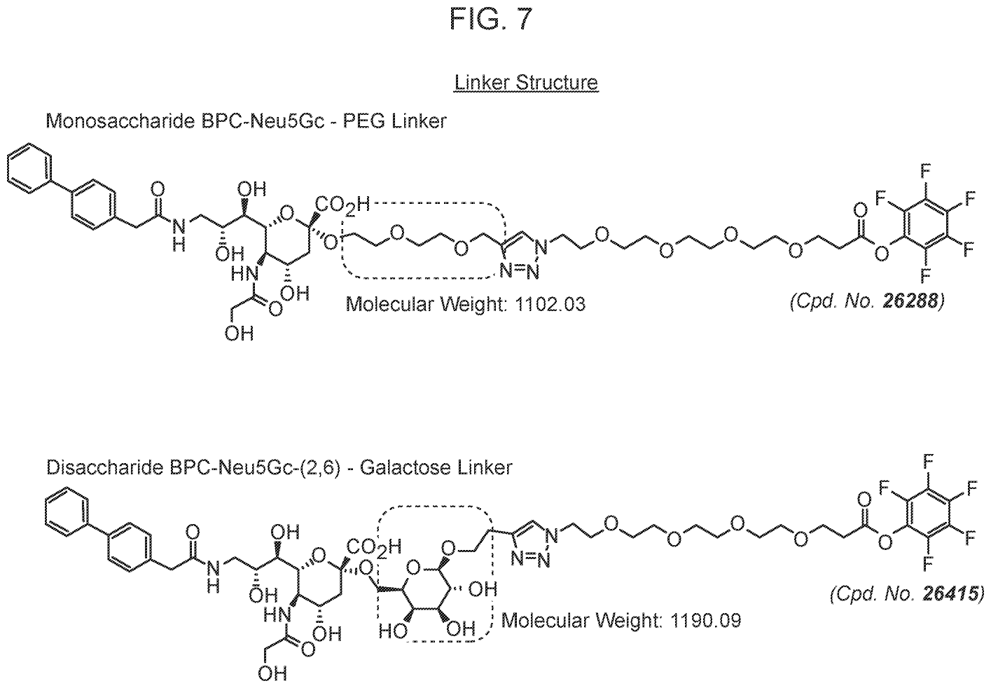

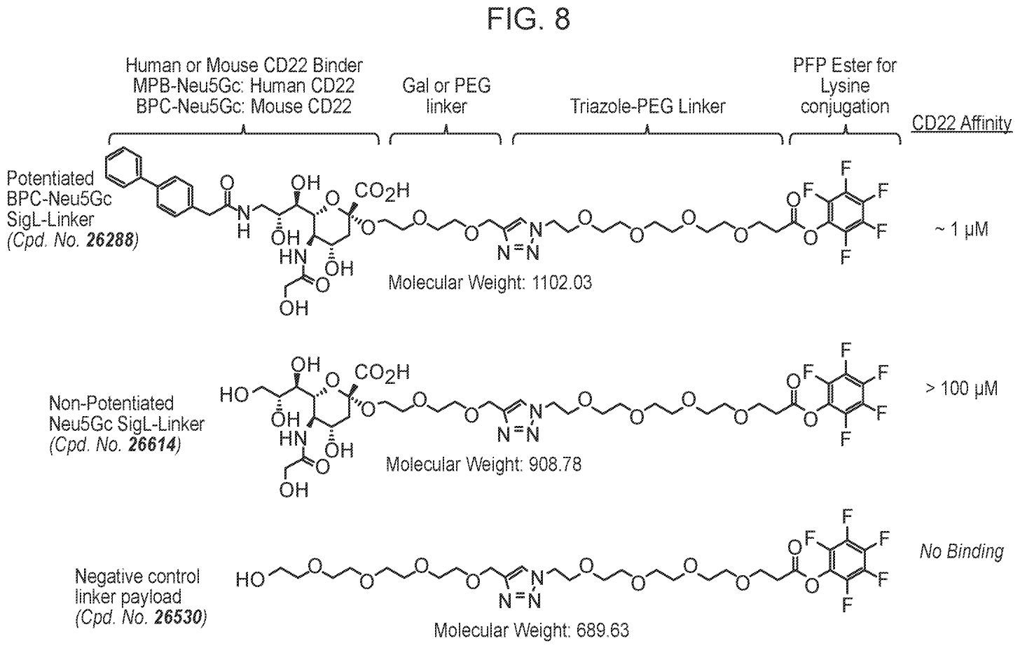

A set of conjugatable linker compounds were designed and synthesized to establish the importance of drug Siglec-2 binding and the importance of potentiated vs non-potentiated Siglec-2 binding for suppression of B cell activation in vitro and anti-drug antibody responses in vivo. FIG. 4 depicts an example conjugatable, CD22-binding, Siglec Ligand-linker structure, highlighting the components of the structure: Siglec Ligand binding moiety, Siglec Ligand-proximal linker structure, Linker, and reactive/conjugatable group. FIG. 5 depicts example Siglec Ligand structures, focusing on elements that determine Siglec-2 binding affinity and species specificity. FIG. 6 depicts example Siglec-2 Ligand structures, showing structures varying in ligand valency. FIG. 7 depicts example Siglec Ligand structures, showing structures varying in linker structure, where a region proximal to the sialic acid-based moiety consists of either a PEG-based structure or a galactose-based structure. FIG. 8 depicts example conjugatable linker structures potentiated for Siglec-2 binding (top), not potentiated for Siglec-2 binding (middle), and an asialo negative control linker structure that does not bind Siglec-2 (bottom). Shown are a potentiated Siglec-ligand linker structure (Cpd. No. 26288, Siglec Ligand: Methyl-α-9-N-(biphenyl-4-carbonyl)-amino-9-deoxy-N-glycolylneuraminic acid) (top), a Siglec ligand-linker structure that contains a non-potentiated Siglec-2 binding moiety (Cpd. No. 26614, Siglec Ligand: N-glycolyl neuraminic acid/Neu5Gc) (middle), and a PEG-based non-Siglec binding conjugatable linker structure (Cpd. No. 26530) (bottom). These compounds were synthesized to high purity and conjugated to different biotherapeutics and proteins, as shown in example 5.

All synthetic chemistry is performed in standard laboratory glassware unless indicated otherwise in the examples. Commercial reagents are used as received. Microwave reactions are performed in an Anton Paar Monowave 400 using the instrument software to control heating time and pressure. Analytical LC/MS is performed either on a Waters Acquity UPLC Instrument with PDA and Single Quadrupole Detector (with alternating positive and negative ion scans) using Masslynx Software or a Shimadzu LCMS-2020 using LabSolutions software. Retention times are determined from the extracted 214 and/or 254 nm UV chromatogram. Prep HPLC is performed either on a Waters Autopurification System consisting of Fraction module 2767, Pump 2545 and 2998 PDA detector using Masslynx software/Agilent 1260 Infinity Autopurification system with DAD detector or on a Gilson system using a 215 liquid handler, 333 and 334 pumps, UV/VIS-155 detector, and Trilution Ic software. 1H NMR is performed either on a Bruker Avance 400 MHz or a Bruker Fourier 300 MHz using Topspin software. Analytical thin layer chromatography is performed on silica (Sigma Aldrich TLC Silica gel 60 F₂₅₄ aluminum or glass TLC plate, silica gel coated with flourescent indicator F254) and is visualized under UV light. Silica gel chromatography is performed manually, or with Teledyne ISCO CombiFlash NextGen 300+ automated chromatography for gradient elution.

### Example 4: Synthesis of Conjugatable Siglec Ligands

### (2R,3S,4S,5R,6R)-2-(acetoxymethyl)-6-(but-3-yn-1-yloxy)tetrahydro-2H-pyran-3,4,5-triyl triacetate (Cpd. No. 26499) and (2R,3R,4S,5S,6R)-2-(but-3-yn-1-yloxy)-6-(hydroxymethyl)tetrahydro-2H-pyran-3,4,5-triyl tribenzoate (Example 1)

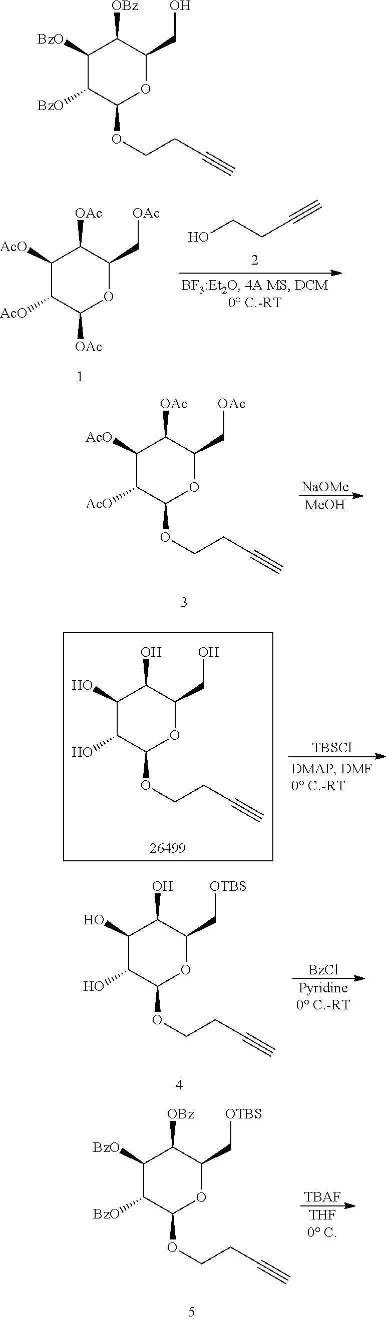

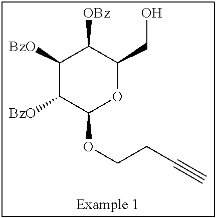

### Synthesis of (2R,3S,4S,5R,6R)-2-(acetoxymethyl)-6-(but-3-yn-1-yloxy)tetrahydro-2H-pyran-3,4,5-triyl triacetate (3)

In an argon atmosphere, (2S,3R,4S,5S,6R)-6-(acetoxymethyl)tetrahydro-2H-pyran-2,3,4,5-tetrayl tetraacetate (1, 10.0 g, 25.62 mmol) and but-3-yn-1-ol (2, 2.69 g, 38.43 mmol) were dissolved in anhydrous dichloromethane (100.0 mL) with stirring. To this solution was added activated powdered 4 Å molecular sieves (10.0 g, 100% w/w). The reaction mixture was stirred at room temperature for 30 min, and then cooled to 0° C. followed by the dropwise addition of boron trifluoride diethyl etherate (22.32 mL, 76.86 mmol) over 30 min. The mixture was stirred at room temperature for 12 h. After completion, the reaction mixture was quenched with triethylamine up to neutral pH, filtered over celite, and washed with dichloromethane (50 mL). To the filtrate was added aqueous sodium bicarbonate (100 mL) with stirring. After 10 min, the organic layer was separated, washed with water (2×50 mL), dried over anhydrous sodium sulfate, and concentrated on a rotary evaporator to obtain a crude residue. The crude residue was purified via column chromatography (60-90% ethyl acetate in hexanes) to afford (2R,3S,4S,5R,6R)-2-(acetoxymethyl)-6-(but-3-yn-1-yloxy)tetrahydro-2H-pyran-3,4,5-triyl triacetate (3) as a white solid. Yield: 8.50 g, 82.87%; ELSD-MS (ESI) m/z 401.38 [M+1]⁺.

### Synthesis of (2R,3S,4S,5R,6R)-2-(acetoxymethyl)-6-(but-3-yn-1-yloxy)tetrahydro-2H-pyran-3,4,5-triyl triacetate (Cpd. No. 26499)

To a stirred solution of (2R,3R,4S,5R,6R)-2-(but-3-yn-1-yloxy)-6-(hydroxymethyl)tetrahydro-2H-pyran-3,4,5-triol (3, 8.5.0 g, 21.23 mmol) in methanol (80 mL) was dropwise added sodium methoxide (25% in methanol) solution (0.44 mL, 2.12 mmol) at 0° C. The reaction mixture was stirred for 1 h at room temperature. After completion, the reaction mixture was cooled to 0° C. and quenched with DOWEX hydrogen form to maintain pH 6. The mixture was filtered through celite and concentrated under reduced pressure to obtain solids that were then triturated with diethyl ether and filtered to afford (2R,3R,4S,5R,6R)-2-(but-3-yn-1-yloxy)-6-(hydroxymethyl)tetrahydro-2H-pyran-3,4,5-triol (Cpd. No. 26499) as an off white solid. Yield: 4.0 g, 81.13%; ELSD-MS (ESI) m/z 250.0 [M+18]⁺.

### Synthesis of (2R,3R,4S,5R,6R)-2-(but-3-yn-1-yloxy)-6-(((tert-butyldimethylsilyl)oxy)methyl)tetrahydro-2H-pyran-3,4,5-triol (4)

To a stirred solution of (2R,3R,4S,5R,6R)-2-(but-3-yn-1-yloxy)-6-(hydroxymethyl)tetrahydro-2H-pyran-3,4,5-triol (26499, 1.0 gm, 4.31 mmol) in dry pyridine (10.0 ml) was added tert-butyl(chloro)dimethylsilane (0.779 gm, 5.17 mmol) at 0° C. The reaction mixture was stirred at room temperature for 16 h. After completion, the reaction mixture was directly concentrated under reduced pressure to obtain a crude residue that was then purified via column chromatography (70-95% ethyl acetate in hexanes) to afford (2R,3R,4S,5R,6R)-2-(but-3-yn-1-yloxy)-6-(((tert-butyldimethylsilyl)oxy)methyl)tetrahydro-2H-pyran-3,4,5-triol (4) as a colorless sticky liquid. Yield; 0.80 g, 53.62%. LCMS m/z 347.0 [M+1]⁺.

### Synthesis of (2R,3R,4S,5S,6R)-2-(but-3-yn-1-yloxy)-6-(((tert-butyldimethylsilyl)oxy)methyl)tetrahydro-2H-pyran-3,4,5-triyl tribenzoate (5)

To a stirred solution of (2R,3R,4S,5R,6R)-2-(but-3-yn-1-yloxy)-6-(((tert-butyldimethylsilyl)oxy)methyl)tetrahydro-2H-pyran-3,4,5-triol (4, 0.75 g, 2.16 mmol) in pyridine (7.5 mL) was added benzoyl chloride (2.01 mL, 17.3 mmol) at 0° C. The mixture was stirred overnight at room temperature. After completion, the reaction mixture was directly concentrated on a rotary evaporator to obtain a crude residue which was then purified via column chromatography (10-30% ethyl acetate in hexanes) to afford (2R,3R,4S,5S,6R)-2-(but-3-yn-1-yloxy)-6-(((tert-butyldimethylsilyl)oxy)methyl)tetrahydro-2H-pyran-3,4,5-triyl tribenzoate (5) as a colorless solid. Yield: 1.25 g, 87.66%; LCMS m/z 659.44 [M+1]⁺.

### Synthesis of (2R,3R,4S,5S,6R)-2-(but-3-yn-1-yloxy)-6-(hydroxymethyl)tetrahydro-2H-pyran-3,4,5-triyl tribenzoate (Example 1)

To a stirred solution of (2R,3R,4S,5S,6R)-2-(but-3-yn-1-yloxy)-6-(((tert-butyldimethylsilyl)oxy)methyl)tetrahydro-2H-pyran-3,4,5-triyl tribenzoate (5, 3.0 g, 4.55 mmol) in oxolane (30.0 ml) was dropwise added tetrabutylazanium fluoride (6.83 mL, 6.83 mmol) at 0° C. The reaction mixture was stirred at 0° C. for 2.5 h. After completion, the reaction mixture was quenched with water and extracted with ethyl acetate. The organic layer was dried over anhydrous sodium sulfate and evaporated under reduced pressure to obtain a crude residue which was then purified via column chromatography (30-40% ethyl acetate in hexanes) to afford (2R,3S,4S,5R,6R)-4,5-bis(acetyloxy)-6-(but-3-yn-1-yloxy)-2-(hydroxymethyl)oxan-3-yl acetate (Example 1) as a white solid. Yield; 1.2 g, 48.39%; ELSD-MS (ESI) m/z 250.0 [M+18]+. ¹H NMR (400 MHz, Methanol-d₄) δ 4.25 (d, J=7.2 Hz, 1H), 3.98-3.92 (m, 3H), 3.81 (d, J=2.8 Hz, 1H), 3.78-3.66 (m, 3H), 3.53-3.43 (m, 3H), 2.50 (dt, J=7.2 & 2.8 Hz, 2H), 2.25 (t, J=2.4 Hz, 1H).

### (1R,2R)-1-((2R,3R,4S,6R)-3-acetamido-4-acetoxy-6-(methoxycarbonyl)-6-(p-tolylthio)tetrahydro-2H-pyran-2-yl)-3-(3-phenoxybenzamido)propane-1,2-diyl diacetate (Example 3)

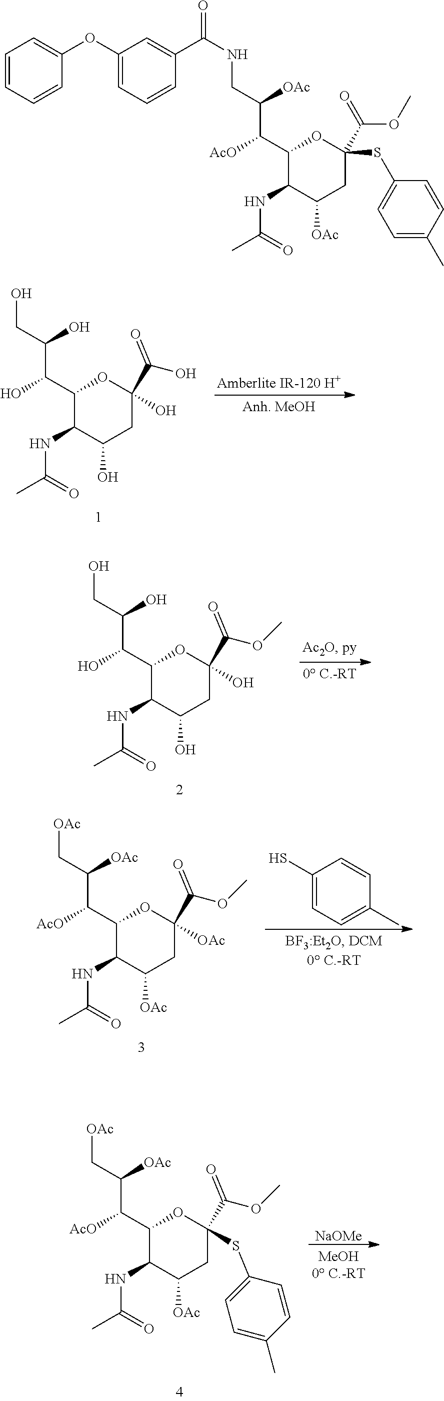

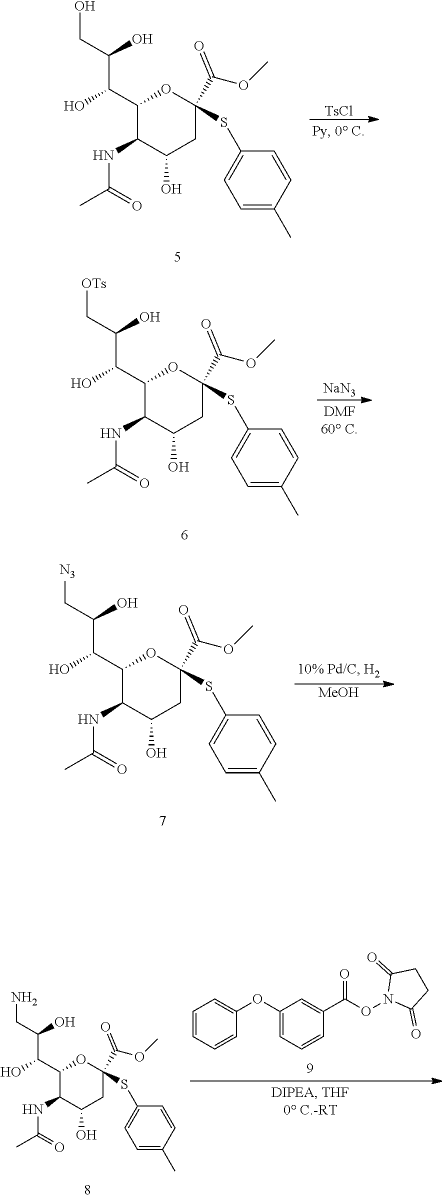

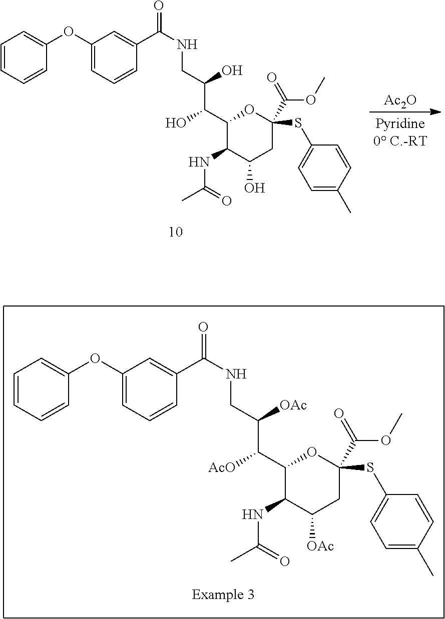 [SMILES: "Ac2O" ; COC(=O)[C@]1(Sc2ccc(C)cc2)C[C@H](O)[C@@H](NC(C)=O)[C@H]([C@H](O)[C@H](O)CNC(=O)c2cccc(Oc3ccccc3)c2)O1 ; COC(=O)[C@]1(Sc2ccc(C)cc2)C[C@H](OC(C)=O)[C@@H](NC(C)=O)[C@H]([C@H](OC(C)=O)[C@@H](CNC(=O)c2cccc(Oc3ccccc3)c2)OC(C)=O)O1]

### Synthesis of methyl (2R,4S,5R,6R)-5-acetamido-2,4-dihydroxy-6-((1R,2R)-1,2,3-trihydroxypropyl)tetrahydro-2H-pyran-2-carboxylate (2)

To a stirred suspension of (2R,4S,5R,6R)-5-acetamido-2,4-dihydroxy-6-((1R,2R)-1,2,3-trihydroxypropyl)tetrahydro-2H-pyran-2-carboxylic acid (1, 100.0 g, 323.3 mmol) in anhydrous methanol (2500 mL) was added Amberlite IR-120 (H⁺) resin (80.0 g) at room temperature under argon atmosphere. The reaction mixture was stirred under inert atmosphere until the suspension became a clear solution. The resin was removed by filtration and the filtrate was concentrated under reduced pressure to obtain a residue. The residue was triturated with diethyl ether and filtered to afford methyl (2R,4S,5R,6R)-5-acetamido-2,4-dihydroxy-6-((1R,2R)-1,2,3-trihydroxypropyl)tetrahydro-2H-pyran-2-carboxylate (2) as a light pink solid. Yield: 104.0 g, 99.49%; LCMS (ESI) m/z 324.2 [M+1]⁺.

### Synthesis of (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3)

In a 2000 mL round bottom flask, methyl (2R,4S,5R,6R)-5-acetamido-2,4-dihydroxy-6-((1R,2R)-1,2,3-trihydroxypropyl)tetrahydro-2H-pyran-2-carboxylate (2, 102.0 g, 315 mmol) was dissolved with stirring in pyridine (600 mL) under argon atmosphere. To this solution was added acetic anhydride (298 mL, 3.15 mmol) dropwise at 0° C. over 30 min under stirring. The mixture was stirred overnight from 0° C. to room temperature. After completion, the reaction mixture was directly concentrated under reduced pressure on a rotary evaporator. The obtained thick syrup was then poured into a separatory funnel with ethyl acetate (500 mL) and washed with aqueous 1N HCl solution (200 mL) followed by saturated sodium bicarbonate (200 mL) solution and DM water (2×200 mL). The organic layer was separated, dried over anhydrous sodium sulfate, and concentrated under reduced pressure to obtain a thick syrup. The syrup was triturated with diethyl ether and filtered to afford (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3) as a white solid. Yield: 130.0 g, 71.83%; LCMS (ESI) m/z 534.2 [M+1]⁺.

### Synthesis of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4)

Under argon atmosphere, (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3, 130.0 g, 243.68 mmol) was dissolved in anhydrous dichloromethane (1300.0 mL) with stirring. To this solution was added activated powdered 4 Å molecular sieves (40.0 g). The reaction mixture was stirred at room temperature for 30 min and cooled to 0° C. followed by the dropwise addition of boron trifluoride diethyl etherate (111.0 mL, 365.5 mmol) over 30 min. The mixture was stirred at room temperature. After completion, the reaction mixture was quenched with triethylamine up to neutral pH, filtered over celite, and washed with dichloromethane (100 mL). To the filtrate was added aqueous sodium bicarbonate (300 mL) with stirring. After 10 min, the organic layer was separated, washed with water (2×300 mL), dried over anhydrous sodium sulfate, and concentrated on a rotary evaporator to obtain a crude residue. The obtained crude residue was purified via column chromatography (60-90% ethyl acetate in hexanes) to afford (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4) as a white solid. Yield: 125.0 g, 85.83%; LCMS (ESI) m/z 598.32 [M+1]⁺.

### Synthesis of methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5)

To a stirred solution of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4, 100.0 g, 167 mmol) in methanol (800 mL) was slowly added sodium methoxide (25% in MeOH) solution (3.58 mL, 16.7 mmol) at 0° C. The reaction mixture was stirred for 2 h at room temperature. After completion, the reaction mixture was cooled to 0° C. and quenched with DOWEX hydrogen form to maintain pH 6. The mixture was filtered through celite and concentrated under reduced pressure to obtain solids that were then triturated with diethyl ether and filtered to afford methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5) as an off white solid. Yield: 71.0 g, 98.80%; LCMS (ESI) m/z 430.10 [M+1]⁺.

### Synthesis of methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6)

To a stirred solution of methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5, 40.0 g, 93.1 mmol) in pyridine (300 mL) was dropwise added a solution of 4-methylbenzene-1-sulfonyl chloride (30.2 g, 158 mmol) in pyridine (100 mL) at 0° C. The resulting reaction solution was stirred overnight. After completion, the reaction mixture was directly concentrated under reduced pressure to obtain a thick syrup. The thick syrup was purified via flash column chromatography (80-95% ethyl acetate in hexanes) to afford methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6) as a white solid. Yield: 21.2 g, 39.0%. LC-MS (ESI) m/z 584.05 [M+1]⁺.

### Synthesis of methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7)

In an inert atmosphere, methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6, 21.0 g, 35.98 mmol) was dissolved under stirring in anhydrous N,N-dimethylformamide (210.0 mL). To this solution was added sodium azide (7.80 g, 120 mmol) at room temperature. The resulting reaction mixture was stirred at 60° C. for 16 h. After completion, the reaction mixture was directly concentrated on a rotary evaporator to obtain a crude solid. The crude solid was purified via column chromatography (80-95% ethyl acetate in hexanes) to afford methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7) as an off white solid. Yield: 11.7 g, 71.55%; LCMS (ESI) m/z 453.19 [M−1]⁻.

### Synthesis of methyl (2R,4S,5R,6R)-5-acetamido-6-((1R,2R)-3-amino-1,2-dihydroxypropyl)-4-hydroxy-2-(p-tolylthio)tetrahydro-2H-pyran-2-carboxylate (8)

To a stirred solution of methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7, 10.0 g, 22.0 mmol) in tetrahydrofuran (100 mL) was added 10% Pd/C (10.0 g, 100% w/w) at room temperature. The reaction was then hydrogenated using a balloon pressure of H₂ gas for 12 h. After completion, the reaction was filtered through celite and the filtrate was concentrated. The residue was dried under vacuum to afford crude methyl (2R,4S,5R,6R)-5-acetamido-6-((1R,2R)-3-amino-1,2-dihydroxypropyl)-4-hydroxy-2-(p-tolylthio)tetrahydro-2H-pyran-2-carboxylate (8) as a thick syrup. Yield 9.9 g, 105.3%; LCMS, m/z 428.16 [M+1]⁺.

### Synthesis of methyl (2R,4S,5R,6R)-5-acetamido-6-((1R,2R)-1,2-dihydroxy-3-(3-phenoxybenzamido)propyl)-4-hydroxy-2-(p-tolylthio)tetrahydro-2H-pyran-2-carboxylate (10)

Methyl (2R,4S,5R,6R)-5-acetamido-6-((1R,2R)-3-amino-1,2-dihydroxypropyl)-4-hydroxy-2-(p-tolylthio)tetrahydro-2H-pyran-2-carboxylate (8, 9.90 g, 21.78 mmol) and 2,5-dioxopyrrolidin-1-yl 3-phenoxybenzoate (9, 7.91 g, 25.41 mmol) were dissolved in tetrahydrofuran (90.0 mL) with stirring under argon atmosphere. To this solution was added ethylbis(propan-2-yl)amine (12.07 mL, 69.31 mmol) at 0° C. The resulting reaction mixture was stirred at room temperature for 12 h. After completion, the reaction mixture was concentrated under reduced pressure to obtain a crude residue which was then purified via column chromatography (80-90% ethyl acetate in hexanes) to afford methyl (2R,4S,5R,6R)-5-acetamido-6-((1R,2R)-1,2-dihydroxy-3-(3-phenoxybenzamido)propyl)-4-hydroxy-2-(p-tolylthio)tetrahydro-2H-pyran-2-carboxylate (10) as a white solid. Yield: 8.10 g, 58.93% (over two steps); LCMS (ESI) m/z 625.23 [M+1]⁺.

### Synthesis of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-phenoxybenzamido)propane-1,2-diyl diacetate (Example 3)

To a stirred solution of methyl (2R,4S,5R,6R)-5-acetamido-6-((1R,2R)-1,2-dihydroxy-3-(3-phenoxybenzamido)propyl)-4-hydroxy-2-(p-tolylthio)tetrahydro-2H-pyran-2-carboxylate (10, 8.0 g, 12.81 mmol) in pyridine (80.0 mL) was added acetic anhydride (5.45 mL, 57.63 mmol) dropwise at 0° C. over 30 min. The mixture was stirred overnight from 0° C. to room temperature. After completion, the reaction mixture was directly concentrated under vacuum. The obtained thick syrup was poured into a separatory funnel with ethyl acetate (80.0 mL) and washed with 1N HCl solution (50 mL) followed by saturated sodium sulfate solution (50 mL) and DM water (2×100 mL). The organic layer was dried over anhydrous sodium sulfate and concentrated under reduced pressure to obtain a crude residue. The residue was purified via column chromatography (55-70% ethyl acetate in hexanes) to afford (1R,2R)-1-((2R,3R,4S,6R)-3-acetamido-4-acetoxy-6-(methoxycarbonyl)-6-(p-tolylthio)tetrahydro-2H-pyran-2-yl)-3-(3-phenoxybenzamido)propane-1,2-diyl diacetate (Example 3) as a white solid. Yield: 4.80 g, 49.92%; LCMS (ESI) m/z 751.25 [M+1]⁺. ¹H NMR (400 MHz, Methanol-d₄) δ 8.20 (d, J=9.6 Hz, 1H), 7.82 (t, J=5.6 Hz, 1H), 7.57-7.35 (m, 7H), 7.17-7.01 (m, 6H), 5.44-5.42 (m, 1H), 5.35 (dt, J=10.4 & 4.8 Hz, 1H), 4.75 (dd, J=10.4 & 1.8 Hz, 1H), 4.03 (d, J=10.4 Hz, 2H), 3.85-3.74 (m, 1H), 3.28-4.26 (m, 1H), 2.61 (dd, J=14.0 & 4.8 Hz, 1H), 2.19 (s, 3H), 2.08-1.96 (m, 7H), 1.85 (s, 3H).

### (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (Example 4)

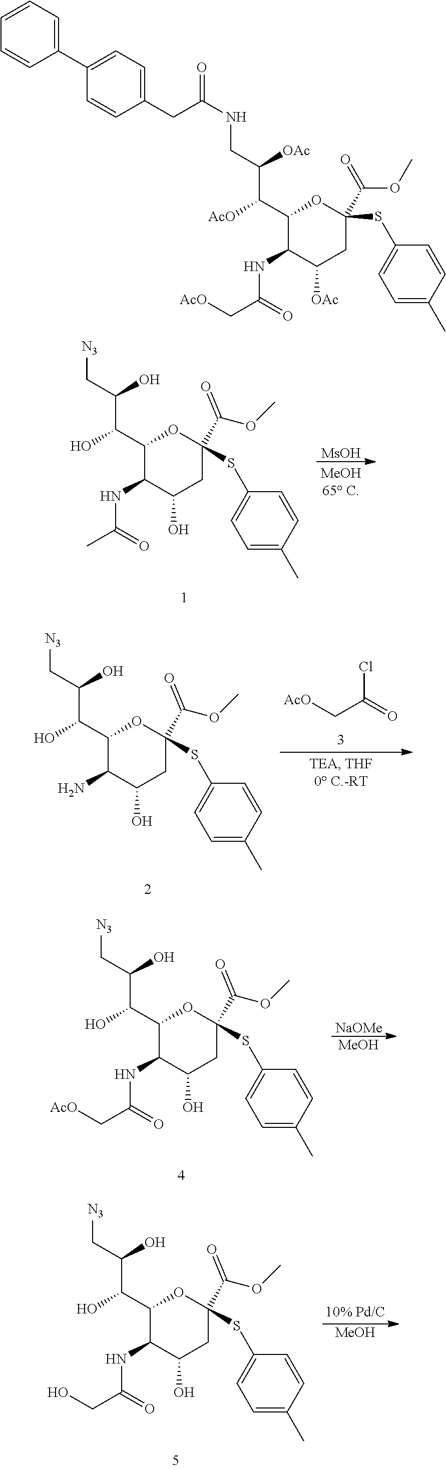

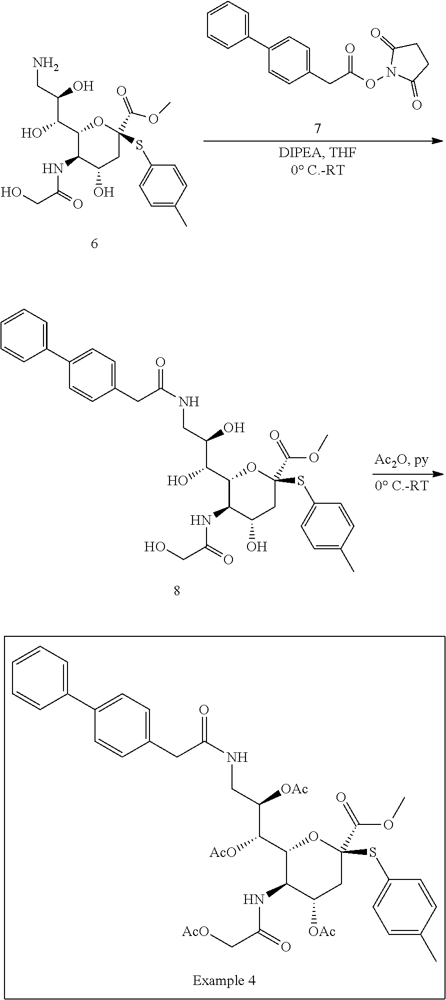

### Synthesis of methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (2)

To a stirred solution of methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (1, 21.0 g, 46.20 mmol) in methanol (210.0 mL) was added methane sulfonic acid (18.0 mL, 277.2 mmol) dropwise at 0° C. The resulting reaction mixture was stirred at 63° C. for 30 h. After completion, the reaction mixture was cooled to 0° C. and quenched with triethylamine (^{˜}15.0 mL, pH 7). The mixture was concentrated under reduced pressure to afford crude methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (2) as a light brown gel. Yield: 19.0 g, 99.68%; LC-MS (ESI) m/z 413.57 [M+1]⁺.

### Synthesis of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (3)

In an inert atmosphere, crude methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (2, 19.0 g, 46.06 mmol) was dissolved under stirring in dry tetrahydrofuran (200.0 mL) and cooled to 0° C. To this solution was slowly added triethylamine (17.70 mL, 138.2 mmol) followed by 2-chloro-2-oxoethyl acetate (4.95 mL, 46.06 mmol) at 0° C. The reaction was stirred at 0° C. until complete. The mixture was concentrated under reduced pressure to obtain a crude residue which was then purified via column chromatography (60-75% ethyl acetate in hexanes) to afford methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (3) as a white solid. Yield: 15.2 g, 64.38%; LCMS (ESI) m/z 513.42 [M+1]⁺.

### Synthesis of methyl (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxylate (4)

To a stirred solution of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (3, 15.0 g, 29.27 mmol) in methanol (150.0 mL) at 0° C. was slowly added sodium methoxide solution (25% in methanol, 0.061 ml, 2.93 mmol). The reaction mixture was stirred for 1 h at room temperature. The reaction mixture was cooled to 0° C. and quenched with DOWEX hydrogen form to maintain pH 6. The mixture was filtered through celite and concentrated under reduced pressure to obtain solids that were triturated with diethyl ether and filtered on a centered funnel to afford methyl (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxylate (4) as an off white solid. Yield: 13.0 g, 94.41%; LCMS (ESI) m/z, 471.15 [M+1]⁺.

### Synthesis of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (5)

To a stirred solution of methyl (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxylate (4, 13.0 g, 27.63 mmol) in methanol (130 mL) was added 10% Pd/C (13.0 g, 100% w/w) at room temperature. The reaction was then hydrogenated using balloon pressure of H₂ gas for 12 h. After completion, the reaction was filtered through celite and the filtrate was concentrated. The obtained residue was then dried under high vacuum to afford crude methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (5) as a thick syrup. Yield: 12.2 g, 99.41%; LCMS (ESI) m/z 445.16 [M+1]⁺.

### Synthesis of methyl (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(p-tolylthio)tetrahydro-2H-pyran-2-carboxylate (7)

To a stirred solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (5, 12.2 g, 27.45 mmol) and 2,5-dioxocyclopentyl 2-([1,1′-biphenyl]-4-yl)acetate (6, 10.19 g, 32.94 mmol) in tetrahydrofuran (40.0 mL) was added ethylbis(propan-2-yl)amine (22.4 mL, 137.23 mmol) at 0° C. The resulting reaction mixture was stirred at room temperature for 12 h. After completion, the mixture was concentrated under reduced pressure to obtain a crude residue. The crude residue was purified via column chromatography (80-90% ethyl acetate in hexanes) to afford methyl (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(p-tolylthio)tetrahydro-2H-pyran-2-carboxylate (7) as a white solid. Yield: 8.0 g, 45.63%; LCMS (ESI) m/z 639.23 [M+1]⁺.

### Synthesis of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (Example 4)

To a stirred solution of methyl (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(p-tolylthio)tetrahydro-2H-pyran-2-carboxylate (7, 8.0 g, 12.52 mmol) in pyridine (80.0 mL) was dropwise added acetic anhydride (11.61 mL, 125.2 mmol) at 0° C. over 30 min. The reaction mixture was stirred overnight from 0° C. to room temperature. After completion, volatiles were removed under vacuum to obtain a crude thick syrup. The crude thick syrup was then poured into a separatory funnel with ethyl acetate (240.0 mL) and washed with 1N HCl solution followed by saturated sodium sulfate solution. The organic layer was dried over anhydrous sodium sulfate and concentrated under reduced pressure to obtain a crude thick syrup. The crude thick syrup was purified via column chromatography (60-70% ethyl acetate in hexanes) to afford (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (Example 4) as a white solid. Yield: 5.40 g, 53.43%; LCMS (ESI) m/z 807.2 [M+1]⁺. ¹H NMR (400 MHz, methanol-d₄) δ 7.27 (d, J=8.4 Hz, 2H), 7.14 (d, J=8.4 Hz, 2H), 4.03 (t, J=8.4 Hz, 1H), 3.77 (d, J=7.6 Hz, 2H), 3.51-3.47 (m, 2H), 3.20 (t, J=6.4 Hz, 2H), 2.91 (dd, J=9.6, 14.4 Hz, 1H), 2.82 (dd, J=6, 14 Hz, 1H), 2.24-2.20 (m, 3H), 2.07 (d, J=9.6 Hz, 1H), 1.75 (d, J=12.8 Hz, 2H), 1.68-1.62 (m, 2H), 1.60-1.57 (m, 2H), 1.56-1.47 (m, 1H).

### (2R,4S,5R,6R)-5-acetamido-6-((1R,2R)-1,2-dihydroxy-3-(3-phenoxybenzamido)propyl)-4-hydroxy-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 24836)

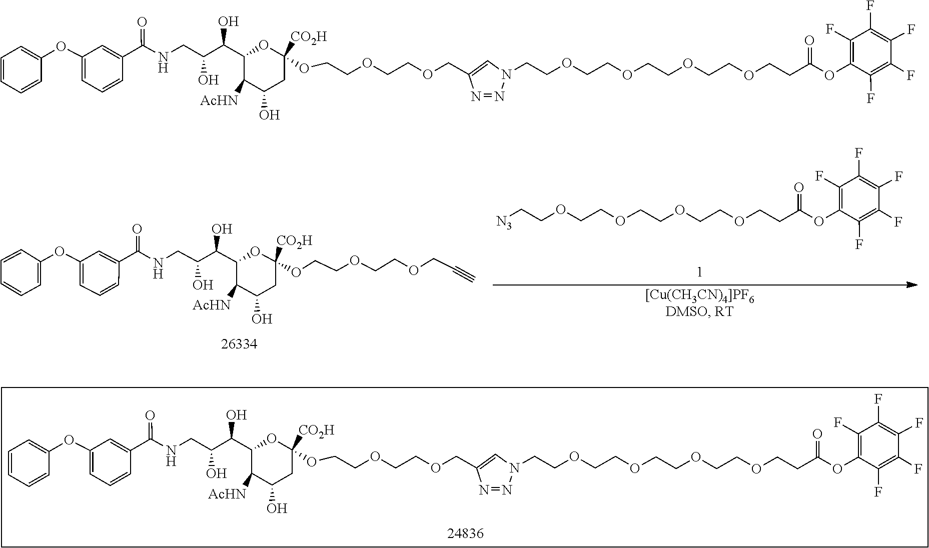

### Synthesis of (2R,4S,5R,6R)-5-acetamido-6-((1R,2R)-1,2-dihydroxy-3-(3-phenoxybenzamido)propyl)-4-hydroxy-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 24836)

To a stirred solution of (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 0.025 g, 0.039 mmol) and perfluorophenyl 1-azido-3,6,9,12-tetraoxapentadecan-15-oate (1, 0.020 g, 0.043 mmol) in dimethyl sulfoxide (2.0 mL) was added tetrakis(acetonitrile)copper(I) hexafluorophosphate (0.041 g, 0.111 mmol). The resulting reaction mixture was stirred at room temperature for 30 min. Thereafter, acetic acid (0.5 mL) was added and the reaction mixture was diluted with acetonitrile and purified via preparatory HPLC (20-42% acetonitrile in water with 0.1% TFA). Fractions containing the desired product were combined and lyophilized to dryness to afford (2R,4S,5R,6R)-5-acetamido-6-((1R,2R)-1,2-dihydroxy-3-(3-phenoxybenzamido)propyl)-4-hydroxy-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 24836) as an amorphous solid. Yield: 0.018 g, 41.73%, LCMS, m/z 1088.53 [M+1]⁺; ¹H NMR (400 MHz, DMSO-d₆ with D₂O exchange) δ 8.00 (d, J=3.2 Hz, 1H), 7.59 (d, J=7.2 Hz, 1H), 7.46-7.36 (m, 4H), 7.16-7.12 (m, 2H), 6.99 (d, J=8.0 Hz, 2H), 4.48-4.45 (m, 4H), 3.76-3.72 (m, 6H), 3.62-3.57 (m, 2H), 3.56-3.43 (m, 23H), 3.24-3.20 (m, 2H), 2.95 (t, J=6.0 Hz, 2H), 2.42-2.39 (m, 1H), 1.84 (s, 3H), 1.53-1.47 (m, 1H).

### (2S,4S,5R,6R)-5-acetamido-6-((1R,2R)-1,2-dihydroxy-3-(3-phenoxybenzamido)propyl)-4-hydroxy-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 24838)

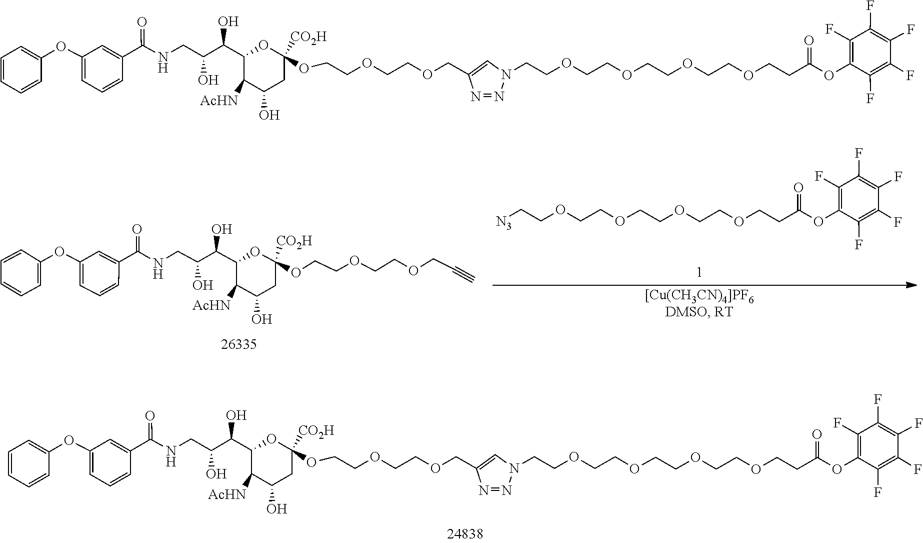

### Synthesis of (2R,4S,5R,6R)-5-acetamido-6-((1R,2R)-1,2-dihydroxy-3-(3-phenoxybenzamido)propyl)-4-hydroxy-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 24838)

To a stirred solution of (2S,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26335, 0.035 g, 0.055 mmol) and perfluorophenyl 1-azido-3,6,9,12-tetraoxapentadecan-15-oate (1, 0.028 g, 0.061 mmol) in dimethyl sulfoxide (2.0 mL) was added tetrakis(acetonitrile)copper(I) hexafluorophosphate (0.057 g, 0.155 mmol). The resulting reaction mixture was stirred at room temperature for 30 min. Acetic acid (0.5 mL) was added and the reaction mixture was diluted with acetonitrile and purified via preparatory HPLC (18-40% acetonitrile in water with 0.1% TFA). Fractions containing the desired product were combined and lyophilized to dryness to afford (2R,4S,5R,6R)-5-acetamido-6-((1R,2R)-1,2-dihydroxy-3-(3-phenoxybenzamido)propyl)-4-hydroxy-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 24838) as an amorphous solid. Yield: 0.017 g, 28.15%, LCMS, m/z 1088.56 [M+1]⁺; ¹H NMR (400 MHz, DMSO-d₆ with D₂O exchange) δ 7.98 (s, 1H), 7.57 (d, J=7.6 Hz, 1H), 7.47-7.36 (m, 4H), 7.16-7.12 (m, 2H), 6.98 (d, J=8.0 Hz, 2H), 4.74-4.44 (m, 4H), 3.76-3.55 (m, 9H), 3.50-3.42 (m, 17H), 3.30-3.17 (m, 3H), 3.04 (dd, J=14.8 & 7.6 Hz, 2H), 2.93 (t, J=6.0 Hz, 2H), 2.16-2.11 (m, 1H), 1.84 (s, 3H), 1.53-1.47 (m, 1H) 1.19-1.13 (m, 3H).

### (2R,4S,5R,6R)-5-acetamido-6-((1R,2R)-1,2-dihydroxy-3-(3-phenoxybenzamido)propyl)-4-hydroxy-2-(2-(2-((1-(15-oxo-3,6,9,12-tetraoxa-16-azaoctadecyl)-1H-1,2,3-triazol-4-yl)methoxy)ethoxy)ethoxy)tetrahydro-2H-pyran-2-carboxylic acid (Cpd. No. 24839)

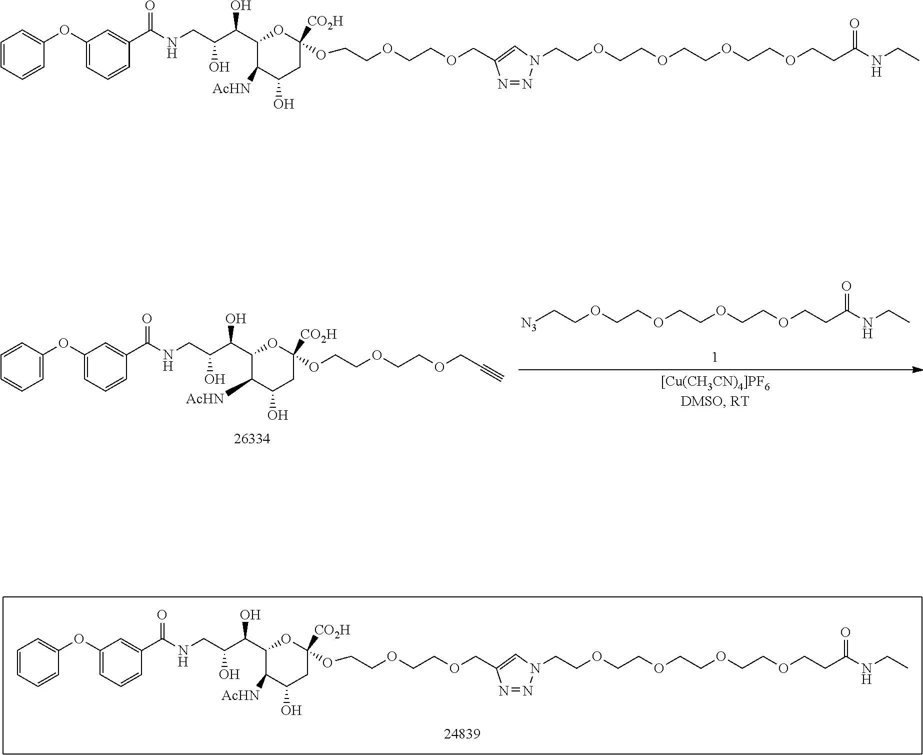 [SMILES: CCNC(=O)CCOCCOCCOCCOCCn1cc(COCCOCCO[C@]2(C(=O)O)C[C@H](O)[C@@H](NC(C)=O)[C@H]([C@H](O)[C@H](O)CNC(=O)c3cccc(Oc4ccccc4)c3)O2)nn1 ; C#CCOCCOCCO[C@]1(C(=O)O)C[C@H](O)[C@@H](NC(C)=O)[C@H]([C@H](O)[C@H](O)CNC(=O)c2cccc(Oc3ccccc3)c2)O1 ; CCNC(=O)CCOCCOCCOCCOCCN=[N+]=[N-] ; CCNC(=O)CCOCCOCCOCCOCCn1cc(COCCOCCO[C@]2(C(=O)O)C[C@H](O)[C@@H](NC(C)=O)[C@H]([C@H](O)[C@H](O)CNC(=O)c3cccc(Oc4ccccc4)c3)O2)nn1]

### Synthesis of (2R,4S,5R,6R)-5-acetamido-6-((1R,2R)-1,2-dihydroxy-3-(3-phenoxybenzamido)propyl)-4-hydroxy-2-(2-(2-((1-(15-oxo-3,6,9,12-tetraoxa-16-azaoctadecyl)-1H-1,2,3-triazol-4-yl)methoxy)ethoxy)ethoxy)tetrahydro-2H-pyran-2-carboxylic acid (Cpd. No. 24839)

To a stirred solution of (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 0.025 g, 0.039 mmol) and 1-azido-N-ethyl-3,6,9,12-tetraoxapentadecan-15-amide (1, 0.014 g, 0.043 mmol) in dimethyl sulfoxide (2.0 mL) was added tetrakis(acetonitrile)copper(I) hexafluorophosphate (0.041 g, 0.111 mmol). The resulting reaction mixture was stirred at room temperature for 30 min. Thereafter, acetic acid (0.5 mL) was added and the reaction mixture was diluted with acetonitrile and purified via preparatory HPLC (20-42% acetonitrile in water with 0.1% TFA). Fractions containing the desired product were combined and lyophilized to dryness to afford (2R,4S,5R,6R)-5-acetamido-6-((1R,2R)-1,2-dihydroxy-3-(3-phenoxybenzamido)propyl)-4-hydroxy-2-(2-(2-((1-(15-oxo-3,6,9,12-tetraoxa-16-azaoctadecyl)-1H-1,2,3-triazol-4-yl)methoxy)ethoxy)ethoxy)tetrahydro-2H-pyran-2-carboxylic acid (Cpd. No. 24839) as an off white solid. Yield: 0.026 g, 60.28%, LCMS, m/z 475.62 [M/2+1]⁺; ¹H NMR (400 MHz, DMSO-d₆) δ 13.84 (bs, 1H), 8.31 (d, J=4.8 Hz, 1H), 8.04 (s, 1H), 7.97 (d, J=6.8 Hz, 1H), 7.80-7.77 (m, 1H), 7.64 (d, J=7.6 Hz, 1H), 7.48-7.38 (m, 4H), 7.18-7.14 (m, 2H), 7.02 (d, J=8.0 Hz, 2H), 4.51-4.48 (m, 4H), 3.81-3.72 (m, 4H), 3.64-3.46 (m, 27H), 3.26-3.16 (m, 1H), 3.07-3.02 (m, 2H), 2.65 (t, J=6.0 Hz, 2H), 1.86 (s, 3H), 1.55-1.49 (m, 1H) 0.98 (t, J=7.2 Hz, 3H). (2S,4S,5R,6R)-5-acetamido-6-((1R,2R)-1,2-dihydroxy-3-(3-phenoxybenzamido)propyl)-4-hydroxy-2-(2-(2-((1-(15-oxo-3,6,9,12-tetraoxa-16-azaoctadecyl)-1H-1,2,3-triazol-4-yl)methoxy)ethoxy)ethoxy)tetrahydro-2H-pyran-2-carboxylic acid (Cpd. No. 24840)

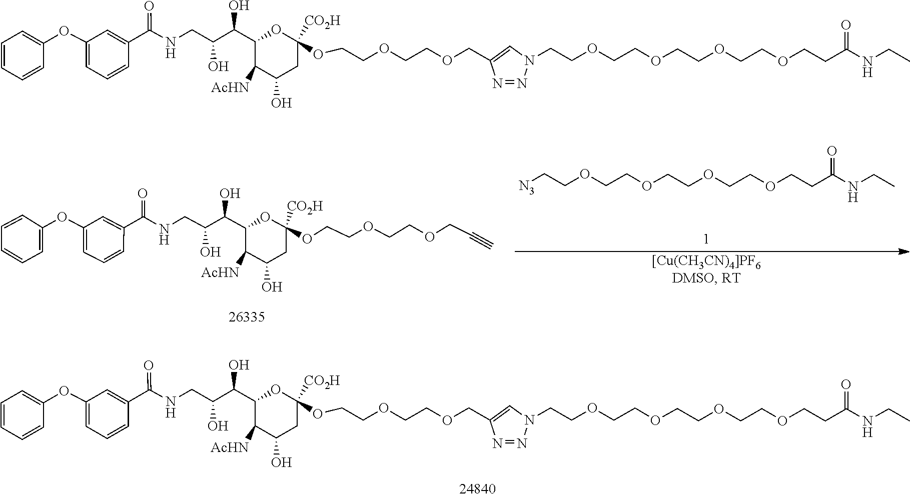

Synthesis of (2S,4S,5R,6R)-5-acetamido-6-((1R,2R)-1,2-dihydroxy-3-(3-phenoxybenzamido)propyl)-4-hydroxy-2-(2-(2-((1-(15-oxo-3,6,9,12-tetraoxa-16-azaoctadecyl)-1H-1,2,3-triazol-4-yl)methoxy)ethoxy)ethoxy)tetrahydro-2H-pyran-2-carboxylic acid (Cpd. No. 24840) To a stirred solution of (2S,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26335, 0.020 g, 0.032 mmol) and 1-azido-N-ethyl-3,6,9,12-tetraoxapentadecan-15-amide (1, 0.011 g, 0.034 mmol) in dimethyl sulfoxide (2.0 mL) was added tetrakis(acetonitrile)copper(I) hexafluorophosphate (0.033 g, 0.088 mmol). The resulting reaction mixture was stirred at room temperature for 30 min. Then, acetic acid (0.5 mL) was added and the reaction mixture was diluted with acetonitrile and purified via preparatory HPLC (18-40% acetonitrile in water with 0.1% TFA). Fractions containing the desired product were combined and lyophilized to dryness to afford (2S,4S,5R,6R)-5-acetamido-6-((1R,2R)-1,2-dihydroxy-3-(3-phenoxybenzamido)propyl)-4-hydroxy-2-(2-(2-((1-(15-oxo-3,6,9,12-tetraoxa-16-azaoctadecyl)-1H-1,2,3-triazol-4-yl)methoxy)ethoxy)ethoxy)tetrahydro-2H-pyran-2-carboxylic acid (Cpd. No. 24840) as an off white solid. Yield: 0.005 g, 14.49%, LCMS, m/z 949.62 [M/2+1]⁺; ¹H NMR (400 MHz, DMSO-d₆ with D₂O exchange) δ 8.00 (s, 1H), 7.59 (d, J=7.6 Hz, 1H), 7.48-7.37 (m, 4H), 7.17-7.13 (m, 2H), 7.00 (d, J=8.0 Hz, 2H), 4.49-4.46 (m, 4H), 3.84-3.73 (m, 5H), 3.62-3.42 (m, 23H), 3.24-3.16 (m, 3H), 3.02 (dd, J=14.4 & 7.2 Hz, 2H), 2.25 (t, J=6.4 Hz, 2H), 2.17-2.12 (m, 1H), 1.84 (s, 3H), 1.51-1.45 (m, 1H) 0.98-0.94 (m, 3H).

### (2R,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 24906)

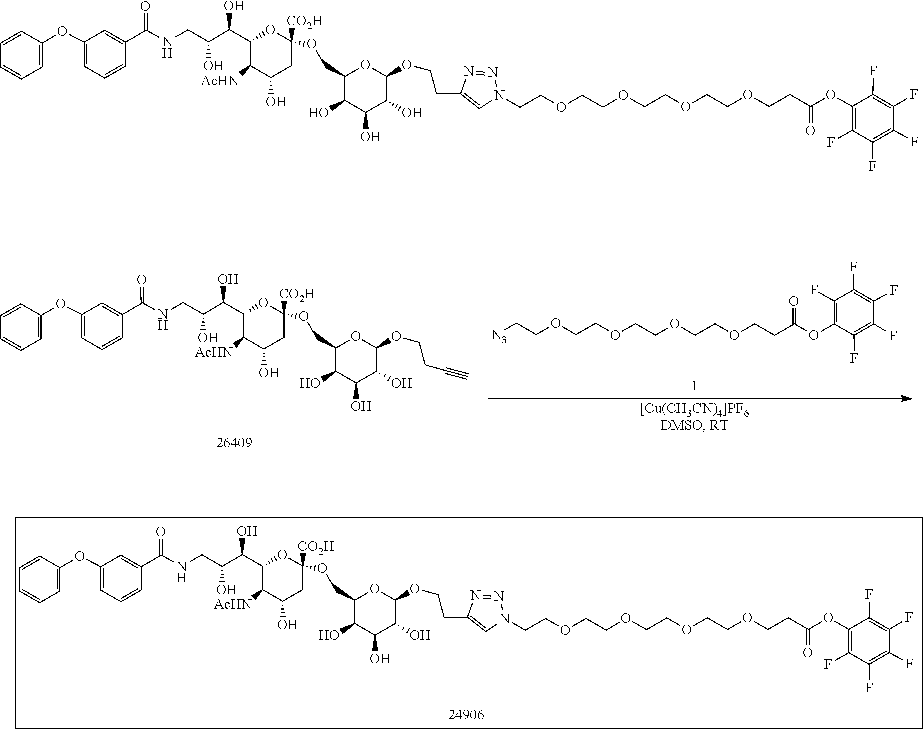

### Synthesis of (2R,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 24906)

To a stirred solution of perfluorophenyl 1-azido-3,6,9,12-tetraoxapentadecan-15-oate (1, 0.156 g, 0.342 mmol) and (2R,4S,5R,6R)-5-acetamido-2-(((2R,3R,4S,5R,6R)-6-(but-3-yn-1-yloxy)-3,4,5-trihydroxytetrahydro-2H-pyran-2-yl) methoxy)-6-((1R,2R)-1,2-dihydroxy-3-(3-phenoxybenzamido)propyl)-4-hydroxytetrahydro-2H-pyran-2-carboxylic acid (26409, 0.087 g, 0.310 mmol) in dimethyl sulfoxide (3 mL) was added tetrakis(acetonitrile)copper(I) hexafluorophosphate (0.324 g, 0.869 mmol). The resulting reaction mixture was stirred at room temperature for 30 min. Thereafter, acetic acid (0.5 mL) was added and the reaction mixture was diluted with acetonitrile and purified via preparatory HPLC (23-41% acetonitrile in water with 0.1% TFA). Fractions containing the desired product were combined and lyophilized to dryness to afford (2-((2R,3S,4R,5S,6R)-3,4,5-trihydroxy-6-((1-(15-oxo-15-(perfluorophenoxy)-3,6,9,12-tetraoxapentadecyl)-1H-1,2,3-triazol-4-yl)methyl)tetrahydro-2H-pyran-2-yl)ethyl)phosphonic acid (Cpd. No. 24906) as an off white solid. Yield: 0.101 g, 44.11%, LCMS (ESI) m/z 738.20 [M+1]⁺; ¹H NMR (400 MHz, DMSO-d₆ with D₂O exchange) δ 4.44 (t, J=5.2 Hz, 2H), 3.89-3.86 (m, 1H), 3.77-3.73 (m, 4H), 3.60-3.56 (m, 2H), 3.53-3.46 (m, 13H), 3.29-3.28 (m, 2H), 2.97 (t, J=5.6 Hz, 2H), 2.86 (d, J=7.2 Hz, 2H), 1.82 (bs, 1H), 1.57 (bs, 1H), 1.46-1.31 (m, 2H).

### (2R,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-3,6,9,12-tetraoxa-16-azaoctadecyl)-1H-1,2,3-triazol-4-yl)ethoxy)tetrahydro-2H-pyran-2-yl)methoxy)tetrahydro-2H-pyran-2-carboxylic acid (Cpd. No. 24924)

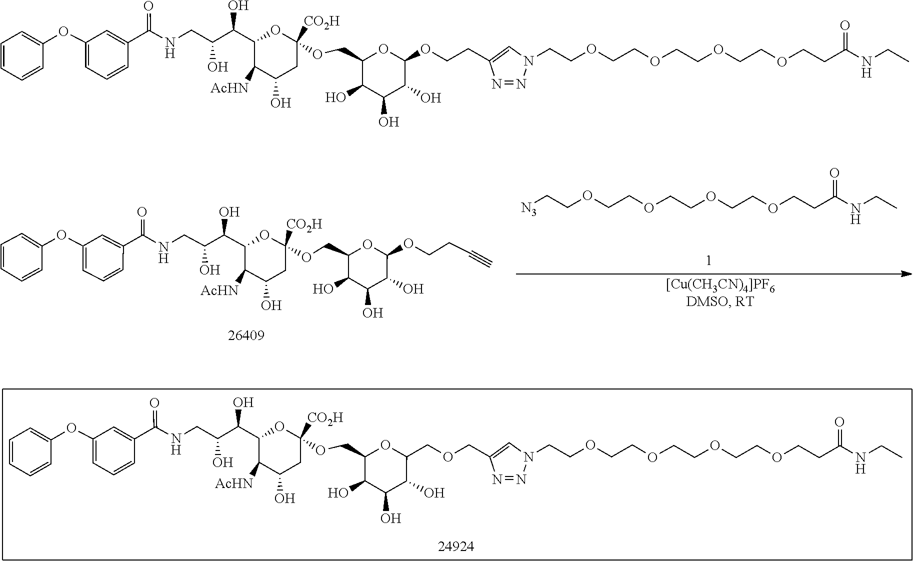

### Synthesis of (2R,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-3,6,9,12-tetraoxa-16-azaoctadecyl)-1H-1,2,3-triazol-4-yl)ethoxy)tetrahydro-2H-pyran-2-yl)methoxy)tetrahydro-2H-pyran-2-carboxylic acid (Cpd. No. 24924)

To a stirred solution of (2R,4S,5R,6R)-5-acetamido-2-(((2R,3R,4S,5R,6R)-6-(but-3-yn-1-yloxy)-3,4,5-trihydroxytetrahydro-2H-pyran-2-yl)methoxy)-6-((1R,2R)-1,2-dihydroxy-3-(3-phenoxybenzamido)propyl)-4-hydroxytetrahydro-2H-pyran-2-carboxylic acid (26409, 0.023 g, 0.032 mmol) and 1-azido-N-ethyl-3,6,9,12-tetraoxapentadecan-15-amide (1, 0.011 g, 0.032 mmol) in dimethyl sulfoxide (2.0 mL) was added tetrakis(acetonitrile)copper(I) hexafluorophosphate (0.029 g, 0.080 mmol). The resulting reaction mixture was stirred at room temperature for 30 min. After completion, acetic acid (0.3 mL) was added and the reaction mixture was diluted with acetonitrile and then purified via preparatory HPLC (15-35% acetonitrile in water with 0.1% TFA). Fractions containing the desired product were combined and lyophilized to dryness to afford (2R,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-3,6,9,12-tetraoxa-16-azaoctadecyl)-1H-1,2,3-triazol-4-yl)ethoxy)tetrahydro-2H-pyran-2-yl)methoxy)tetrahydro-2H-pyran-2-carboxylic acid (Cpd. No. 24924) as the TFA salt as a white solid. Yield: 0.006 g, 18.08%; LCMS (ESI) m/z 1037.75 [M+1]⁺; ¹H NMR (400 MHz, DMSO-d₆ with D2O exchange) δ 7.86 (s, 1H), 7.59 (d, J=8.0 Hz, 1H), 7.46-7.37 (m, 4H), 7.16-7.11 (m, 2H), 6.99 (d, J=7.6 Hz, 1H), 4.42 (t, J=5.0 Hz, 2H), 4.13 (d, J=6.8 Hz, 1H), 3.91-3.85 (m, 1H), 3.79-3.73 (m, 6H), 3.63-3.60 (m, 2H), 3.56-3.52 (m, 5H), 3.49-3.43 (m, 15H), 3.27-3.21 (m, 4H), 3.02 (ABq, J=7.6 Hz, 2H), 2.83-2.80 (m, 2H), 2.25 (t, J=6.4 Hz, 1H), 1.83 (s, 3H), 1.57-1.51 (m, 1H), 0.96 (t, J=7.2 Hz, 3H).

### (2R,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26288)

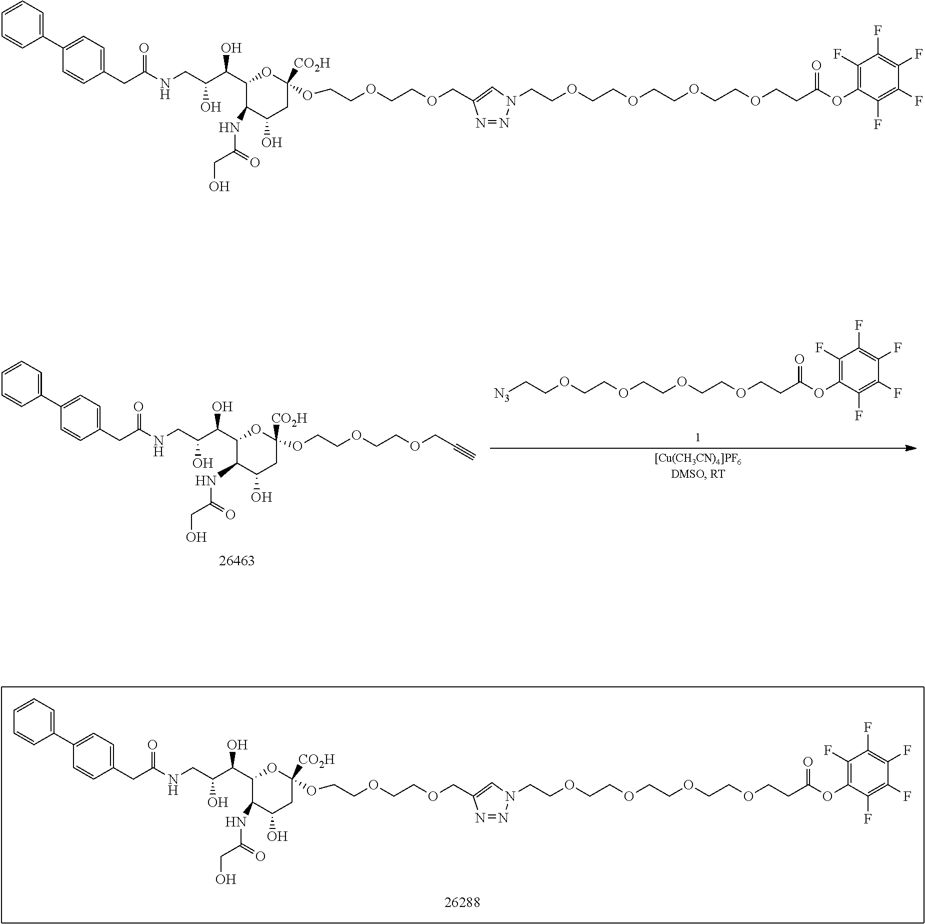 [SMILES: O=C(Cc1ccc(-c2ccccc2)cc1)NC[C@@H](O)[C@@H](O)[C@@H]1O[C@@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO ; C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1 ; [N-]=[N+]=NCCOCCOCCOCCOCCC(=O)Oc1c(F)c(F)c(F)c(F)c1F ; O=C(Cc1ccc(-c2ccccc2)cc1)NC[C@@H](O)[C@@H](O)[C@@H]1O[C@@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO]

### Synthesis of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 26288)

To a stirred solution of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 0.035 g, 0.054 mmol) and perfluorophenyl 1-azido-3,6,9,12-tetraoxapentadecan-15-oate (1, 0.025 g, 0.054 mmol) in dimethyl sulfoxide (0.5 mL) was added tetrakis(acetonitrile)copper(I) hexafluorophosphate (0.050 g, 0.135 mmol). The resulting reaction mixture was stirred at room temperature for 30 min. After completion, acetic acid (0.3 mL) was added. The resulting solution was diluted with acetonitrile and purified via preparatory HPLC (19-35% acetonitrile in water with 0.1% TFA). Fractions containing the desired product were combined and lyophilized to dryness to afford (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 26288) as the TFA salt as a white solid. Yield: 0.022 g, 36.77%; LCMS (ESI) m/z 1102.72 [M+1]⁺; ¹H NMR (400 MHz, DMSO-d₆ with D₂O exchange) δ 7.98 (d, J=3.6 Hz, 2H), 7.59 (d, J=7.2 Hz, 2H), 7.56-7.52 (m, 2H), 7.42 (t, J=8.0 Hz, 2H), 7.33-7.31 (m, 3H), 4.46 (s, 4H), 4.11 (m, 2H), 3.55-3.40 (m, 22H), 3.19 (d, J=9.2 Hz, 2H), 2.99-2.92 (m, 5H), 2.45-2.43 (m, 6H), 2.50 (m, 1H), 1.20 (s, 1H).

### (2R,2′R,4S,4′S,5R,5′R,6R,6′R)-2,2′-(((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(5-acetamido-6-((1R,2R)-1,2-dihydroxy-3-(3-phenoxybenzamido)propyl)-4-hydroxytetrahydro-2H-pyran-2-carboxylic acid) (Cpd. No. 26289)

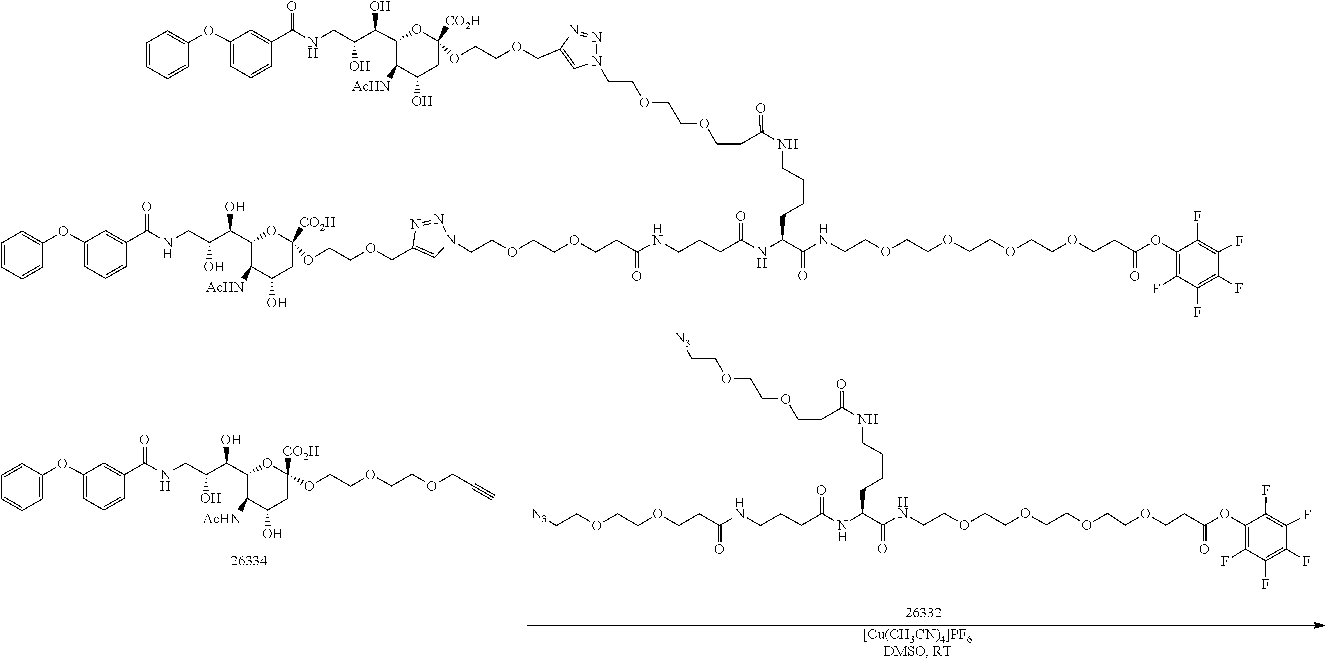

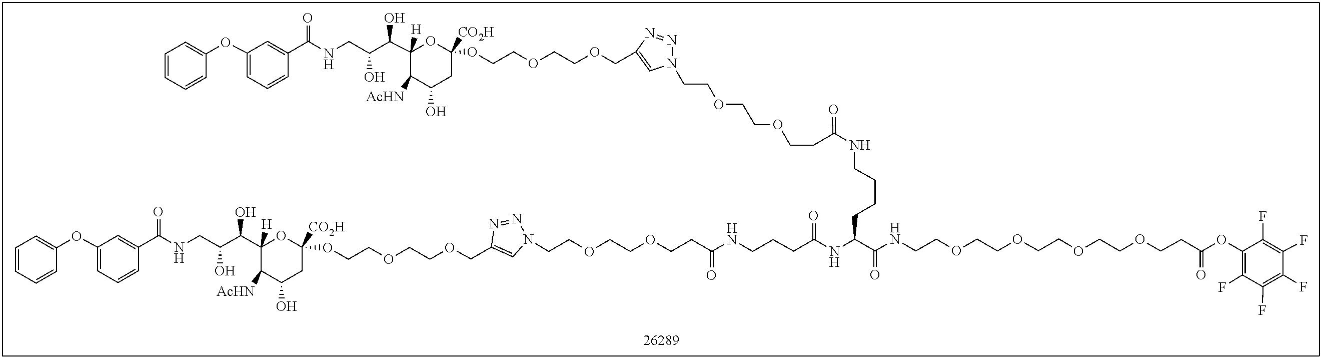 [SMILES: CC(=O)N[C@H]1[C@H]([C@H](O)[C@H](O)CNC(=O)c2cccc(Oc3ccccc3)c2)O[C@@](OCCOCCOCc2cn(CCOCCOCCC(=O)NCCCC[C@H](NC(=O)CCCNC(=O)CCOCCOCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)NCCOCCOCCOCCOCCC(=O)Oc3c(F)c(F)c(F)c(F)c3F)nn2)(C(=O)O)C[C@@H]1O]

### Synthesis of (2R,2′R,4S,4′S,5R,5′R,6R,6′R)-2,2′-(((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(5-acetamido-6-((1R,2R)-1,2-dihydroxy-3-(3-phenoxybenzamido)propyl)-4-hydroxytetrahydro-2H-pyran-2-carboxylic acid) (Cpd. No. 26289)

To a stirred solution of (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 0.025 g, 0.039 mmol) and perfluorophenyl (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 0.020 g, 0.019 mmol) in dimethyl sulfoxide (0.5 mL) was added tetrakis(acetonitrile)copper(I) hexafluorophosphate (0.036 g, 0.099 mmol). The resulting reaction mixture was stirred at room temperature for 30 min. The progress of the reaction was monitored by LC-MS and after completion, acetic acid (0.3 mL) was added. The resulting solution was diluted with acetonitrile and purified via preparatory HPLC (25-44% acetonitrile in water with 0.1% TFA). Fractions containing the desired product were combined and lyophilized to dryness to afford (2R,2′R,4S,4′S,5R,5′R,6R,6′R)-2,2′-(((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(5-acetamido-6-((1R,2R)-1,2-dihydroxy-3-(3-phenoxybenzamido)propyl)-4-hydroxytetrahydro-2H-pyran-2-carboxylic acid) (Cpd. No. 26289) as the TFA salt as a white solid. Yield: 0.012 g, 27.67%; LCMS (ESI) m/z 738.20 [M+1]⁺; ¹H NMR (400 MHz, DMSO-d₆ with D₂O exchange) δ 7.98 (s, 2H), 7.58 (d, J=8.0 Hz, 2H), 7.46-7.36 (m, 9H), 7.16-7.12 (m, 4H), 6.98 (d, J=7.6 Hz, 4H), 4.46-4.43 (m, 9H), 4.11 (m, 2H), 3.53-3.35 (m, 56H), 3.23-3.21 (m, 5H), 3.14 (bs, 2H), 2.97-2.93 (m, 9H), 2.25-2.22 (m, 6H), 2.08 (bs, 4H), 1.83 (s, 6H), 1.56-1.44 (m, 8H), 1.49-1.44 (m, 2H), 1.33-1.27 (m, 4H), 1.20 (bs, 5H), 0.98 (d, J=6.8 Hz, 2H).

### Perfluorophenyl (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (Cpd. No. 26332)

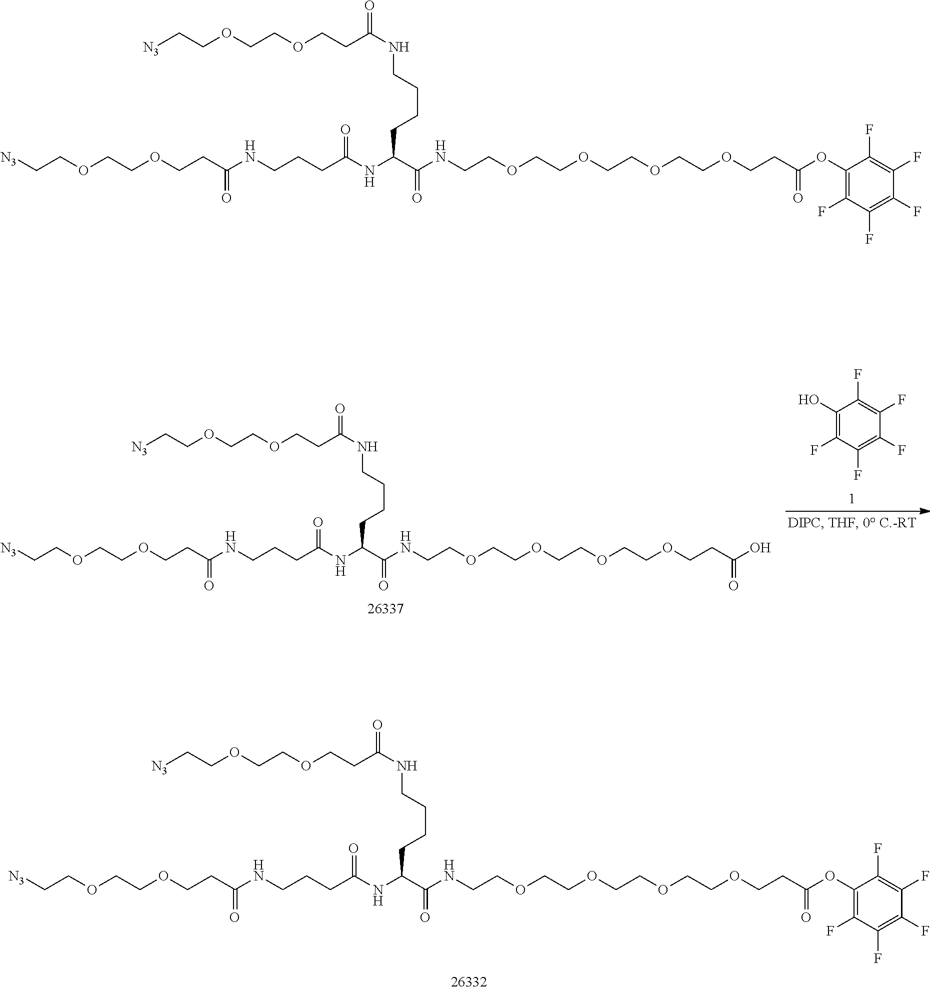 [SMILES: [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)O ; Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)Oc1c(F)c(F)c(F)c(F)c1F]

### Synthesis of Perfluorophenyl (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (Cpd. No. 26332)

To a stirred solution of (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oic acid (26337, 1.0 g, 1.18 mmol) in tetrahydrofuran (10.0 mL) at 0° C. was added 2,3,4,5,6-pentafluorophenol (1, 0.434 g, 2.36 mmol) and N, N′-diisopropylcarbodiimide (0.461 mL, 2.94 mmol). The resulting reaction mixture was stirred at room temperature for 24 h. After completion, the solvent was concentrated and purified via preparatory HPLC (30-75% acetonitrile in water with 0.1% TFA). Fractions containing the desired product were combined and lyophilized to dryness to afford Perfluorophenyl(S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (Cpd. No. 26332) as an off white solid. Yield: 0.291 g, 24%; ELSD (ESI) m/z 1015.6 [M+1]⁺; ¹H NMR (400 MHz, DMSO-d₆) δ 7.92 (t, J=11.6 Hz, 1H), 7.85-7.80 (m, 2H), 7.78 (t, J=11.2 Hz, 1H), 4.19-4.14 (m, 1H), 3.78 (t, J=12.0 Hz, 2H), 3.61-3.49 (m, 29H), 3.38-3.35 (m, 5H), 3.25-3.13 (m, 3H), 3.07-2.99 (m, 6H), 2.32-2.26 (m, 4H), 1.62-1.20 (m, 9H).

### Perfluorophenyl (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (Cpd. No. 26333)

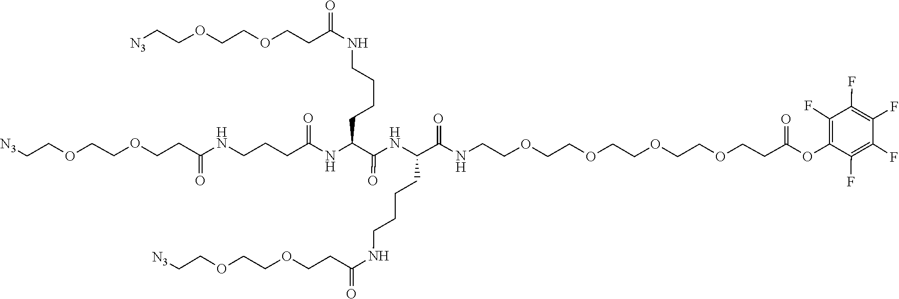 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F]

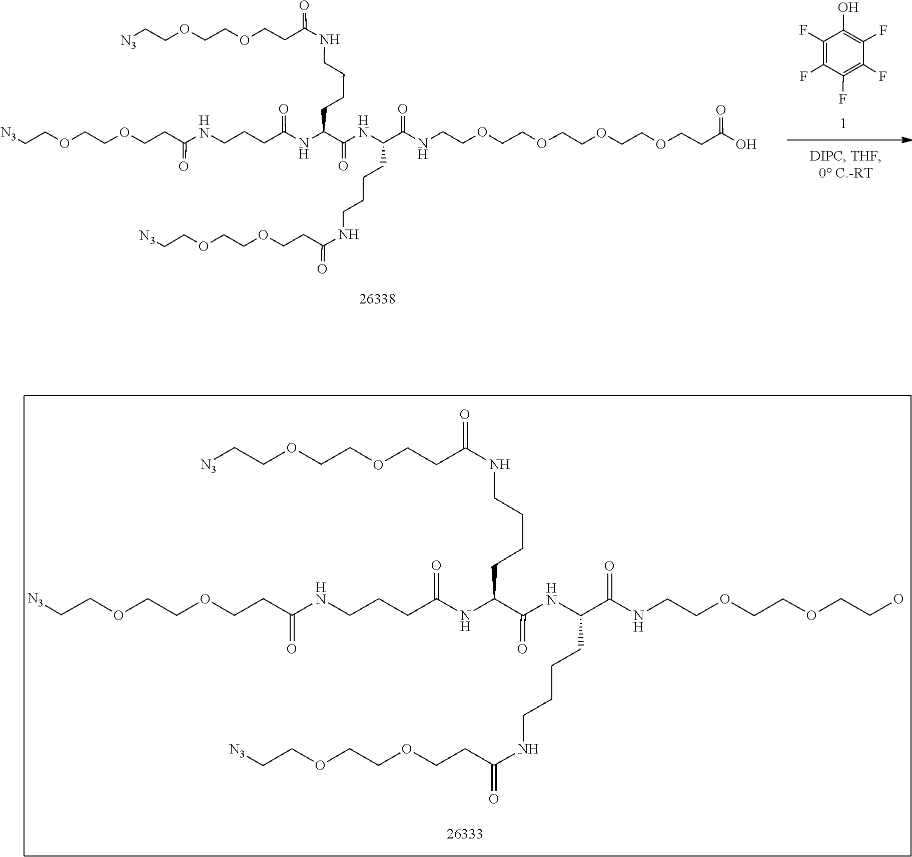

### Synthesis of Perfluorophenyl (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (Cpd. No. 26333)

To a stirred solution of (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oic acid (26338, 1.5 g, 1.29 mmol) in tetrahydrofuran (20.0 mL) at 0° C. was added 2,3,4,5,6-pentafluorophenol (1, 0.475 g, 2.58 mmol) and N, N′-diisopropylcarbodiimide (0.472 mL, 3.22 mmol). The resulting reaction mixture was stirred at room temperature for 24 h. After completion, the solvent was concentrated to obtain a residue which was then purified via preparatory HPLC (20-55% acetonitrile in water with 0.1% TFA). The desired fractions were combined and lyophilized to dryness to afford Perfluorophenyl (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (Cpd. No. 26333) as an off white sticky solid. Yield: 0.556 g, 32.25%; LC-MS (ESI) m/z 1329.09 [M+1]^{+ 1}H NMR (400 MHz, DMSO-d₆) δ 7.98-7.68 (m, 6H), 4.16 (bs, 2H), 3.78 (t, J=11.6 Hz, 2H), 3.59-3.49 (m, 35H), 3.38-3.33 (m, 9H), 3.21-3.17 (m, 4H), 3.03-2.99 (m, 9H), 2.30-2.27 (m, 6H), 2.14 (t, J=14.4 Hz, 2H), 1.60-1.57 (m, 4H), 1.48-1.23 (m, 10H).

### (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (Cpd. No. 26334) and (2S,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (Cpd. No. 26335)

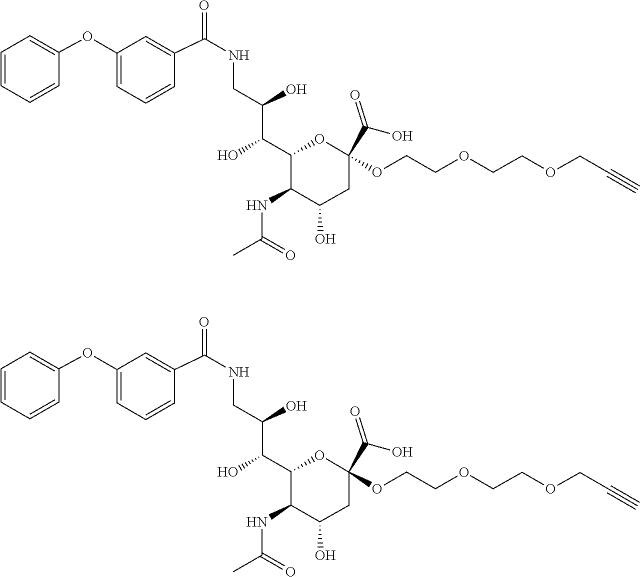

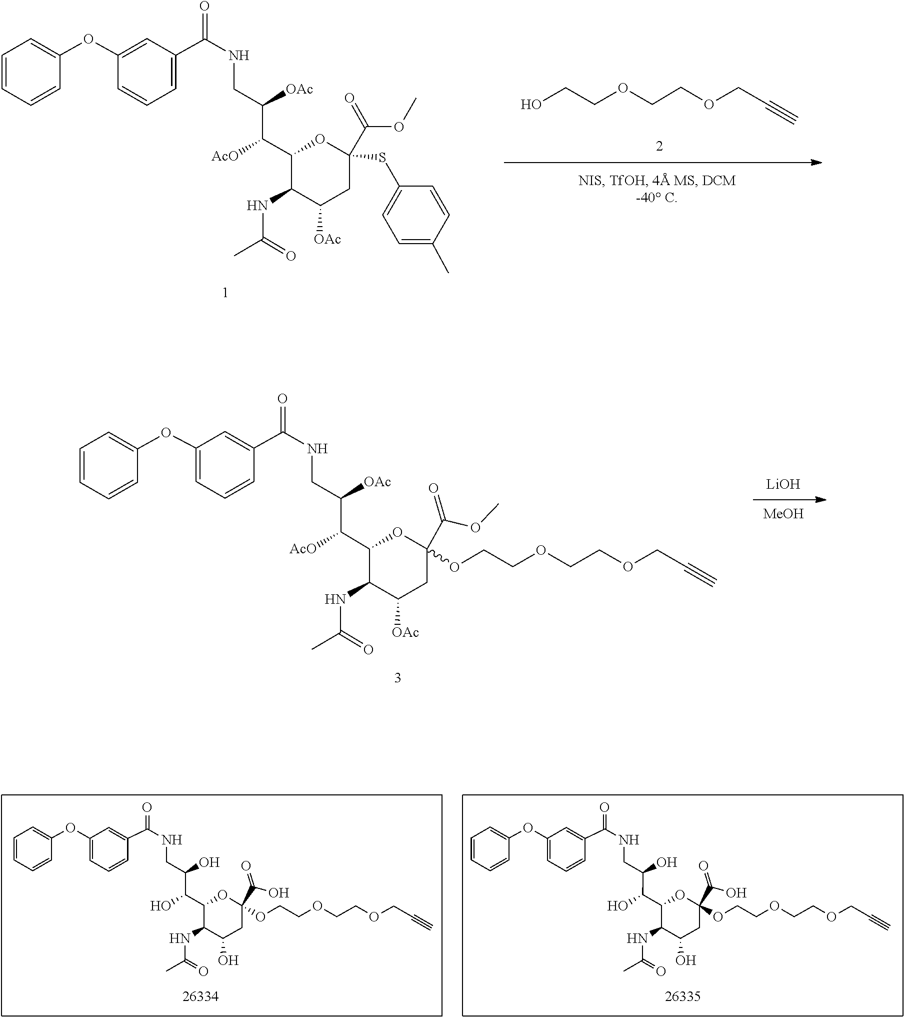

### Synthesis of (1R,2R)-1-((2R,3R,4S,6R)-3-acetamido-4-acetoxy-6-(methoxycarbonyl)-6-(2-(2-(prop-2-yn-1-yloxy)ethoxy)ethoxy)tetrahydro-2H-pyran-2-yl)-3-(3-phenoxybenzamido)propane-1,2-diyl diacetate (3)

To a stirred solution of (1R,2R)-1-((2R,3R,4S,6S)-3-acetamido-4-acetoxy-6-(methoxycarbonyl)-6-(p-tolylthio)tetrahydro-2H-pyran-2-yl)-3-(3-phenoxybenzamido)propane-1,2-diyl diacetate (1, 1.0 g, 1.33 mmol) and 2-(2-(prop-2-yn-1-yloxy)ethoxy)ethan-1-ol (2, 0.288 g, 2.0 mmol) in dry dichloromethane (20.0 mL) was added 4 Å Molecular Sieves (1.0 g, 100% w/w). The resulting reaction mixture was stirred under nitrogen atmosphere for 12 h. Then, 1-iodopyrrolidine-2,5-dione (0.576 g, 2.56 mmol) and trifluoromethanesulfonic acid (0.094 mL, 1.066 mmol) were added at −45° C. The resulting reaction mixture was stirred at −45° C. for 1 h. After completion, the reaction mixture was quenched with trimethylamine up to neutral pH, filtered through celite, diluted with dichloromethane, and washed with aqueous sodium bicarbonate solution followed by DM water. The organic layer was concentrated to obtain a crude residue which was then purified via flash column chromatography (5-7.5% methanol in ethyl acetate). The desired fractions were concentrated and dried to afford (1R,2R)-1-((2R,3R,4S,6R)-3-acetamido-4-acetoxy-6-(methoxycarbonyl)-6-(2-(2-(prop-2-yn-1-yloxy)ethoxy)ethoxy)tetrahydro-2H-pyran-2-yl)-3-(3-phenoxybenzamido)propane-1,2-diyl diacetate (3) as a white solid as an anomeric mixture. Yield: 1.0 g, 97.41%; LCMS (ESI) m/z 771.79.

### Synthesis of (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (Cpd. No. 26334) and (2S,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (Cpd. No. 26335)

To a stirred solution of (1R,2R)-1-((2R,3R,4S,6R)-3-acetamido-4-acetoxy-6-(methoxycarbonyl)-6-(2-(2-(prop-2-yn-1-yloxy)ethoxy)ethoxy)tetrahydro-2H-pyran-2-yl)-3-(3-phenoxybenzamido)propane-1,2-diyl diacetate (3, 1.0 g, 1.302 mmol) in ethanol (10.0 mL) was added lithium hydroxide (0.186 g, 7.78 mmol) at 0° C. The resulting reaction mixture was stirred at room temperature for 4 h. After completion, Dowex-Hydrogen form was added up to pH 6 and the reaction mass was filtered. The filtrate was concentrated and dried to obtain a crude residue which was then diluted with acetonitrile and purified via preparatory HPLC (22-38% acetonitrile in water with 0.1% TFA). Fractions containing the desired products were separately combined and lyophilized to dryness to afford (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (Cpd. No. 26334) as the TFA salt as a thick syrup. Yield: 0.24 g, 29.33%; LCMS (ESI) m/z 631.55 [M+1]⁺; ¹H NMR (400 MHz, DMSO-d₆) δ 8.36 (t, J=5.4 Hz, 1H), 8.06 (d, J=7.6 Hz, 1H), 7.65 (d, J=8.0 Hz, 1H), 7.50-7.39 (m, 4H), 7.17-7.15 (m, 2H), 7.02 (d, J=8.0 Hz, 2H), 4.83 (bs, 2H), 4.13 (d J=2.0 Hz, 1H), 3.76-3.71 (m, 4H), 3.59-3.14 (m, 14H), 2.17 (dd, J=12.8 & 4.8 Hz, 1H), 1.87 (s, 3H), 1.49 (t, J=12.0 Hz, 1H); (2S,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (Cpd. No. 26335) as the TFA salt as a white solid. Yield: 0.23 g, 28.11%; LCMS (ESI) m/z 631.55 [M+1]⁺; ¹H NMR (400 MHz, DMSO-d₆) δ 8.32 (t, J=5.2 Hz, 1H), 7.99 (d, J=7.2 Hz, 1H), 7.64 (d, J=7.6 Hz, 1H), 7.48-7.39 (m, 4H), 7.18-7.14 (m, 2H), 7.30 (d, J=6.8 Hz, 2H), 4.33 (bs, 1H), 4.11 (d J=2.0 Hz, 1H), 3.82-3.73 (m, 2H), 3.66-3.24 (m, 11H), 3.25-3.21 (m, 1H), 1.88 (s, 3H), 1.52 (t, J=12.0 Hz, 1H).

### (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oic acid (Cpd. No. 26337)

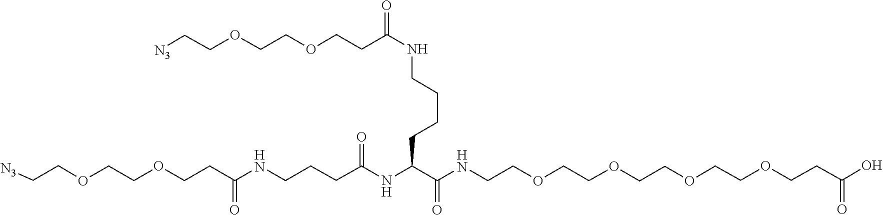 [SMILES: [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)O]

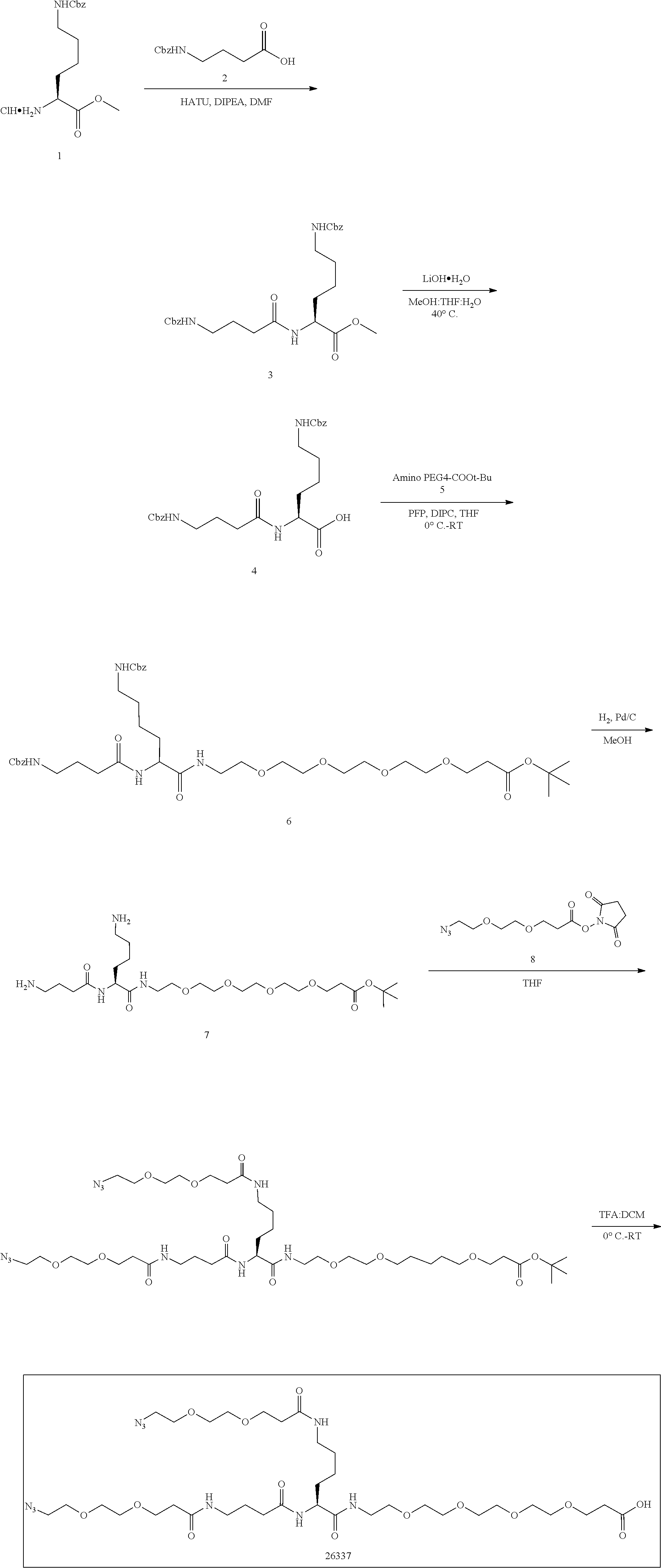

### Synthesis of methyl N6-((benzyloxy)carbonyl)-N2-(4-(((benzyloxy)carbonyl)amino)butanoyl)-L-lysinate (3)

To a solution of methyl N6-((benzyloxy)carbonyl)-L-lysinate hydrochloride (1, 5.00 g, 19.5 mmol) was added 4-(((benzyloxy)carbonyl)amino)butanoic acid (2, 4.63 g, 17.0 mmol) in N,N-dimethylformamide (50.0 mL), [(dimethylamino)({3H-[1,2,3]triazolo[4,5-b]pyridin-3-yloxy})methylidene]dimethylazanium; hexafluoro-λ⁵-phosphanuide (8.07 g, 21.2 mmol) and diisopropylethylamine (11.0 mL, 59.5 mmol). The resulting reaction mixture was stirred at room temperature for 8 h. After completion, the reaction mixture was diluted with saturated sodium bicarbonate solution and extracted with dichloromethane. The organic layer was dried over sodium sulfate, filtered, and concentrated under high vacuum to obtain a crude residue which was purified via flash column chromatography (50-70% ethyl acetate in hexanes). Desired fractions were concentrated under reduced pressure to afford methyl N6-((benzyloxy)carbonyl)-N2-(4-(((benzyloxy)carbonyl)amino)butanoyl)-L-lysinate (3) as an off white solid. Yield: 7.0 g, 90.25%; LC-MS (ESI) m/z 514.2 [M+1]+.

Synthesis of N6-((benzyloxy)carbonyl)-N2-(4-(((benzyloxy)carbonyl)amino)butanoyl)-L-lysine (4) To a solution of methyl N6-((benzyloxy)carbonyl)-N2-(4-(((benzyloxy)carbonyl)amino)butanoyl)-L-lysinate (3, 7.0 g, 13.6 mmol) in methanol (25.0 mL), tetrahydrofuran (25.0 mL), and water (5.0 mL) was added lithium hydroxide (0.979 g, 40.9 mmol) at room temperature. The resulting mixture was stirred at 40° C. for 4 h. After completion, the reaction mixture was acidified with 1N HCl solution (pH 4) and extracted with ethyl acetate. The organic layer was dried over sodium sulfate, filtered, and concentrated under high vacuum to afford crude N6-((benzyloxy)carbonyl)-N2-(4-(((benzyloxy)carbonyl)amino)butanoyl)-L-lysine (4) as an off white solid. Yield: 6.0 g, 94.25%; LCMS (ESI) m/z 500.2 [M+1]⁺.

### Synthesis of tert-butyl (S)-10-(4-(((benzyloxy)carbonyl)amino)butyl)-3,8,11-trioxo-1-phenyl-2,15,18,21,24-pentaoxa-4,9,12-triazaheptacosan-27-oate (6)

To a solution of N6-((benzyloxy)carbonyl)-N2-(4-(((benzyloxy)carbonyl)amino)butanoyl)-L-lysine (4, 3.0 g, 6.01 mmol) in tetrahydrofuran (30.0 mL) at 0° C. was added 2,3,4,5,6-pentafluorophenol (2.21 g, 12.0 mmol) and N, N′-diisopropylcarbodiimide (1.91 mL, 15.0 mmol). The resulting reaction mixture was stirred at room temperature for 4 h. Tert-butyl 1-amino-3,6,9,12-tetraoxapentadecan-15-oate (5, 2.32 g, 7.21 mmol) was added at room temperature and the reaction mixture was stirred for 12 h. After completion, the solvent was concentrated under high vacuum to obtain a crude residue which was then purified via flash column chromatography (70-100% ethyl acetate in hexanes). Desired fractions were concentrated under reduced pressure to afford tert-butyl (S)-10-(4-(((benzyloxy)carbonyl)amino)butyl)-3,8,11-trioxo-1-phenyl-2,15,18,21,24-pentaoxa-4,9,12-triazaheptacosan-27-oate (6) as a pale yellow sticky Yield: 4.0 g, 84.25%; LC-MS (ESI) m/z 803.41 [M+1]⁺.

### Synthesis of tert-butyl (S)-23-amino-18-(4-aminobutyl)-17,20-dioxo-4,7,10,13-tetraoxa-16,19-diazatricosanoate (7)

To a stirred solution of tert-butyl (S)-10-(4-(((benzyloxy)carbonyl)amino)butyl)-3,8,11-trioxo-1-phenyl-2,15,18,21,24-pentaoxa-4,9,12-triazaheptacosan-27-oate (6, 4.0 g, 4.98 mmol) in Methanol (50 mL) was added 10% palladium on carbon (4.0 g, 100% w/w) at room temperature under nitrogen. The resulting mixture was stirred at room temperature under hydrogen gas pressure for 12 h. The reaction mixture was filtered through celite and washed with methanol. The filtrate was concentrated under vacuum to afford tert-butyl (S)-23-amino-18-(4-aminobutyl)-17,20-dioxo-4,7,10,13-tetraoxa-16,19-diazatricosanoate (7) as an off white solid. Yield: 2.0 g, 75%; ELSD (ESI) m/z 803.4 [M+1]⁺.

### Synthesis of tert-butyl (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (9)

To a solution of tert-butyl (S)-23-amino-18-(4-aminobutyl)-17,20-dioxo-4,7,10,13-tetraoxa-16,19-diazatricosanoate (7, 2.0 g, 3.74 mmol) in tetrahydrofuran (40.0 mL) at 0° C. was added 2,5-dioxopyrrolidin-1-yl 3-(2-(2-azidoethoxy)ethoxy)propanoate (2.25 g, 7.48 mmol). The resulting reaction mixture was stirred at room temperature for 4 h. After completion, the solvent was concentrated under high vacuum to obtain a crude residue which was then purified via flash column chromatography (0-7.5% methanol in dichloromethane). Desired fractions were concentrated under reduced pressure to afford tert-butyl (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (9) as a pale yellow viscous liquid Yield: 1.10 g, 32.25%; ELSD (ESI) m/z 905.4 [M+1]⁺.

### Synthesis of (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oic acid (Cpd. No. 26337)

To a solution of tert-butyl (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (9, 1.1 g, 1.21 mmol) in dichloromethane (10 mL) was added trifluoroacetic acid (5.0 mL) at 0° C. The resulting mixture was stirred at room temperature under nitrogen for 4 h. After completion, the reaction mixture was concentrated, washed with diethyl ether, and dried to afford (S)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oic acid (Cpd. No. 26337) as a pale yellow viscous liquid. Yield: 1.0 g, 99.25%; ELSD (ESI) m/z 849.6 [M+1]⁺. ¹H NMR (400 MHz, DMSO-d₆) δ 12.0 (bs, 1H), 7.92 (t, J=11.6 Hz, 1H), 7.85-7.80 (m, 2H), 7.78 (t, J=11.2 Hz, 1H), 4.20-4.15 (m, 1H), 3.63-3.49 (m, 31H), 3.40-3.36 (m, 5H), 3.25-3.17 (m, 2H), 3.15-3.07 (m, 4H), 2.45-2.42 (m, 2H), 2.32-2.26 (m, 4H), 2.13 (t, J=14.8 Hz, 2H), 1.62-1.14 (m, 8H).

### (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oic acid (Cpd. No. 26338)

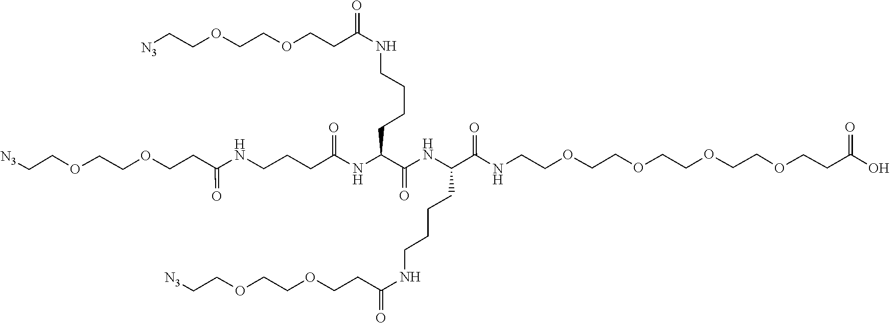 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)O]

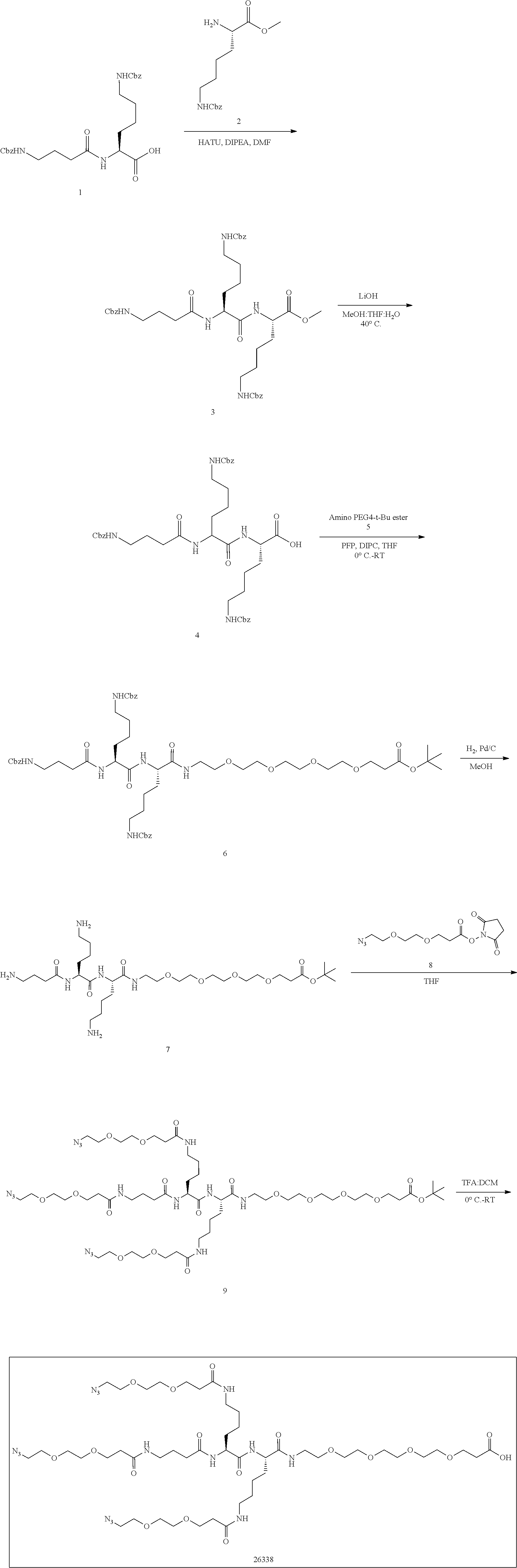

### Synthesis of methyl N6-((benzyloxy)carbonyl)-N2-(N6-((benzyloxy)carbonyl)-N2-(4-(((benzyloxy)carbonyl)amino)butanoyl)-L-lysyl)-L-lysinate (3)

To a stirred solution of N6-((benzyloxy)carbonyl)-N2-(4-(((benzyloxy)carbonyl)amino)butanoyl)-L-lysine (1, 6.0 g, 12.0 mmol) in N,N-dimethylformamide (50.0 mL) at 0° C. was added methyl N6-((benzyloxy)carbonyl)-L-lysinate (2, 4.24 g, 14.4 mmol) followed by [(dimethylamino)({3H-[1,2,3]triazolo[4,5-b]pyridin-3-yloxy})methylidene]dimethylazanium; hexafluoro-λ⁵-phosphanuide (9.59 g, 25.2 mmol) and ethylbis(propan-2-yl)amine (7.32 mL, 42.0 mmol). The resulting reaction mixture was stirred at room temperature for 12 h. After completion, the reaction mixture was diluted with saturated sodium bicarbonate solution and extracted with ethyl acetate. The organic layer was dried over sodium sulfate, filtered, and concentrated under high vacuum to obtain a crude residue. The crude residue was purified via flash column chromatography (50-70% ethyl acetate in hexanes) to afford methyl N6-((benzyloxy)carbonyl)-N2-(N6-((benzyloxy)carbonyl)-N2-(4-(((benzyloxy)carbonyl)amino)butanoyl)-L-lysyl)-L-lysinate (3) as an off white solid. Yield: 9.0 g, 96.25%; LCMS (ESI) m/z 776.54 [M+1]⁺.

### Synthesis of N6-((benzyloxy)carbonyl)-N2-(N6-((benzyloxy)carbonyl)-N2-(4-(((benzyloxy)carbonyl)amino)butanoyl)-L-lysyl)-L-lysine (4)

To a stirred solution of methyl N6-((benzyloxy)carbonyl)-N2-(N6-((benzyloxy)carbonyl)-N2-(4-(((benzyloxy)carbonyl)amino)butanoyl)-L-lysyl)-L-lysinate (3, 9.0 g, 11.6 mmol) in methanol (30.0 mL) and tetrahydrofuran (15.0 mL) was added lithium hydroxide (0.833 g, 34.8 mmol) dissolved in water (1.0 mL). The resulting mixture was stirred at 40° C. for 16 h. After completion, the reaction mixture was concentrated and the crude residue was dissolved in water and acidified with 1N hydrochloric acid solution up to pH 4 and extracted with ethyl acetate. The organic layer was separated, dried over anhydrous sodium sulfate, and concentrated under reduced pressure to afford N6-((benzyloxy)carbonyl)-N2-(N6-((benzyloxy)carbonyl)-N2-(4-(((benzyloxy)carbonyl)amino)butanoyl)-L-lysyl)-L-lysine (4) as an off white solid. Yield: 8.0 g, 90%; LCMS (ESI) m/z 762.43 [M+1]⁺.

### Synthesis of tert-butyl (10S,13S)-10,13-bis(4-(((benzyloxy)carbonyl)amino)butyl)-3,8,11,14-tetraoxo-1-phenyl-2,18,21,24,27-pentaoxa-4,9,12,15-tetraazatriacontan-30-oate (6)

To a stirred solution of N6-((benzyloxy)carbonyl)-N2-(N6-((benzyloxy)carbonyl)-N2-(4-(((benzyloxy)carbonyl)amino)butanoyl)-L-lysyl)-L-lysine (4, 4.0 g, 5.25 mmol) in tetrahydrofuran (40.0 mL) at 0° C. was added 2,3,4,5,6-pentafluorophenol (1.93 g, 12.0 mmol) and N, N′-diisopropylcarbodiimide (1.67 mL, 13.1 mmol). The resulting reaction mixture was stirred at room temperature for 4 h. To the reaction mixture was added tert-butyl 1-amino-3,6,9,12-tetraoxapentadecan-15-oate (5, 2.03 g, 6.30 mmol). The resulting reaction mixture was stirred at room temperature for 12 h. After completion, the solvent was concentrated under high vacuum to afford a crude residue which was then purified via flash column chromatography (70-100% ethyl acetate in hexanes). The desired fractions were concentrated under reduced pressure to afford tert-butyl (10S,13S)-10,13-bis(4-(((benzyloxy)carbonyl)amino)butyl)-3,8,11,14-tetraoxo-1-phenyl-2,18,21,24,27-pentaoxa-4,9,12,15-tetraazatriacontan-30-oate (6) as a pale yellow semi-solid. Yield: 5.0 g, 89.25%; LC-MS (ESI) m/z 1065.8 [M+1]⁺.

### Synthesis of tert-butyl (18S,21S)-26-amino-18,21-bis(4-aminobutyl)-17,20,23-trioxo-4,7,10,13-tetraoxa-16,19,22-triazahexacosanoate (7)

To a stirred solution of tert-butyl (10S,13S)-10,13-bis(4-(((benzyloxy)carbonyl)amino)butyl)-3,8,11,14-tetraoxo-1-phenyl-2,18,21,24,27-pentaoxa-4,9,12,15-tetraazatriacontan-30-oate (6, 5.0 g, 4.69 mmol) in methanol (40.0 mL) was added 10% palladium on carbon (5.0 g, 100% w/w) at room temperature under nitrogen. The resulting mixture was stirred at room temperature under hydrogen gas pressure for 12 h. The reaction mixture was filtered through celite and washed with methanol. The filtrate was concentrated under vacuum to afford tert-butyl (18S,21S)-26-amino-18,21-bis(4-aminobutyl)-17,20,23-trioxo-4,7,10,13-tetraoxa-16,19,22-triazahexacosanoate (7) as a pale yellow viscous liquid. Yield: 3.0 g, 96.23%; ELSD (ESI) m/z 663.5 [M+1]⁺.

### Synthesis of tert-butyl (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (9)

To a stirred solution of tert-butyl (18S,21S)-26-amino-18,21-bis(4-aminobutyl)-17,20,23-trioxo-4,7,10,13-tetraoxa-16,19,22-triazahexacosanoate (7, 3.0 g, 4.53 mmol) in tetrahydrofuran (30.0 mL) at 0° C. was added 2,5-dioxopyrrolidin-1-yl 3-(2-(2-azidoethoxy)ethoxy)propanoate (8, 4.08 g, 13.6 mmol). The resulting reaction mixture was stirred at room temperature for 4 h. After completion, the solvent was concentrated under high vacuum to obtain a crude residue which was then purified via flash column chromatography (0-7.5% methanol in dichloromethane). The desired fractions were concentrated under reduced pressure to afford tert-butyl (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (9) as a pale yellow viscous liquid Yield: 2.2 g, 39.30%; ELSD (ESI) m/z 1217.6 [M+1]⁺.

### Synthesis of (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oic acid (Cpd. No. 26338)

To a stirred solution of tert-butyl (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (9, 2.2 g, 1.80 mmol) in dichloromethane (20 mL) was added trifluoroacetic acid (5.0 mL) at 0° C. The resulting reaction mixture was stirred at room temperature under nitrogen for 4 h. After completion, the reaction mixture was concentrated, washed with diethyl ether, and dried to afford (16S,19S)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oic acid (Cpd. No. 26338) as an off white viscous liquid. Yield: 1.4 g, 70.35%; ELSD (ESI) m/z 1161.4 [M−1]⁻. ¹H NMR (400 MHz, DMSO-d₆) δ 12.0 (bs, 1H), 7.97-7.77 (m, 5H), 4.16 (bs, 2H), 3.60-3.57 (m, 14H), 3.53-3.48 (m, 24H), 3.38-3.19 (m, 14H), 2.94 (bs, 9H), 2.43-2.42 (m, 8H), 2.30-2.27 (m, 5H), 2.14 (t, J=15.2 Hz, 2H), 1.62-1.59 (m, 4H), 1.49-1.48 (m, 2H), 1.37-1.23 (m, 8H).

### (2R,4S,5R,6R)-6-((1R,2R)-3-(2-([1,1′-biphenyl]-4-yl)acetamido)-1,2-dihydroxypropyl)-4-acetoxy-2-(((2R,3R,4S,5R,6R)-6-(but-3-yn-1-yloxy)-3,4,5-trihydroxytetrahydro-2H-pyran-2-yl)methoxy)-5-(2-hydroxyacetamido)tetrahydro-2H-pyran-2-carboxylic acid (Cpd. No. 26339) and (2S,4S,5R,6R)-6-((1R,2R)-3-(2-([1,1′-biphenyl]-4-yl)acetamido)-1,2-dihydroxypropyl)-4-acetoxy-2-(((2R,3R,4S,5R,6R)-6-(but-3-yn-1-yloxy)-3,4,5-trihydroxytetrahydro-2H-pyran-2-yl)methoxy)-5-(2-hydroxyacetamido)tetrahydro-2H-pyran-2-carboxylic acid (Cpd. No. 26411)

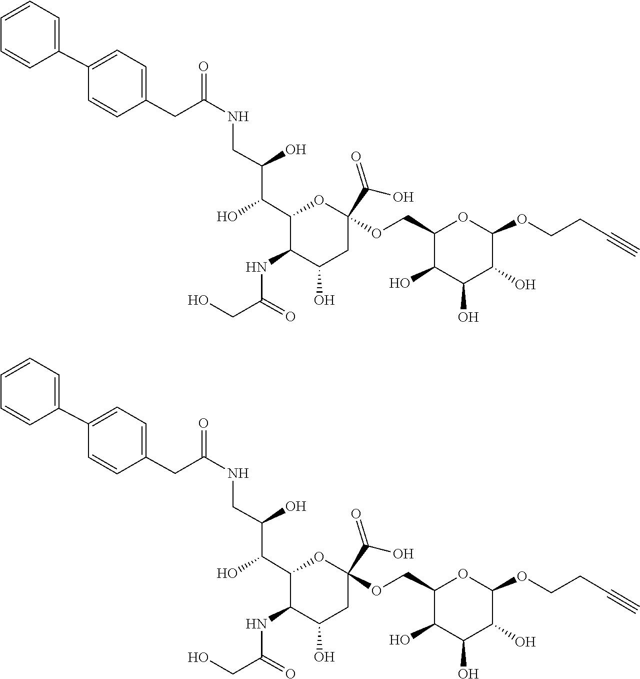 [SMILES: C#CCCO[C@@H]1O[C@H](CO[C@]2(C(=O)O)C[C@H](O)[C@@H](NC(=O)CO)[C@H]([C@H](O)[C@H](O)CNC(=O)Cc3ccc(-c4ccccc4)cc3)O2)[C@H](O)[C@H](O)[C@H]1O ; C#CCCO[C@@H]1O[C@H](CO[C@]2(C(=O)O)C[C@H](O)[C@@H](NC(=O)CO)[C@H]([C@H](O)[C@H](O)CNC(=O)Cc3ccc(-c4ccccc4)cc3)O2)[C@H](O)[C@H](O)[C@H]1O]

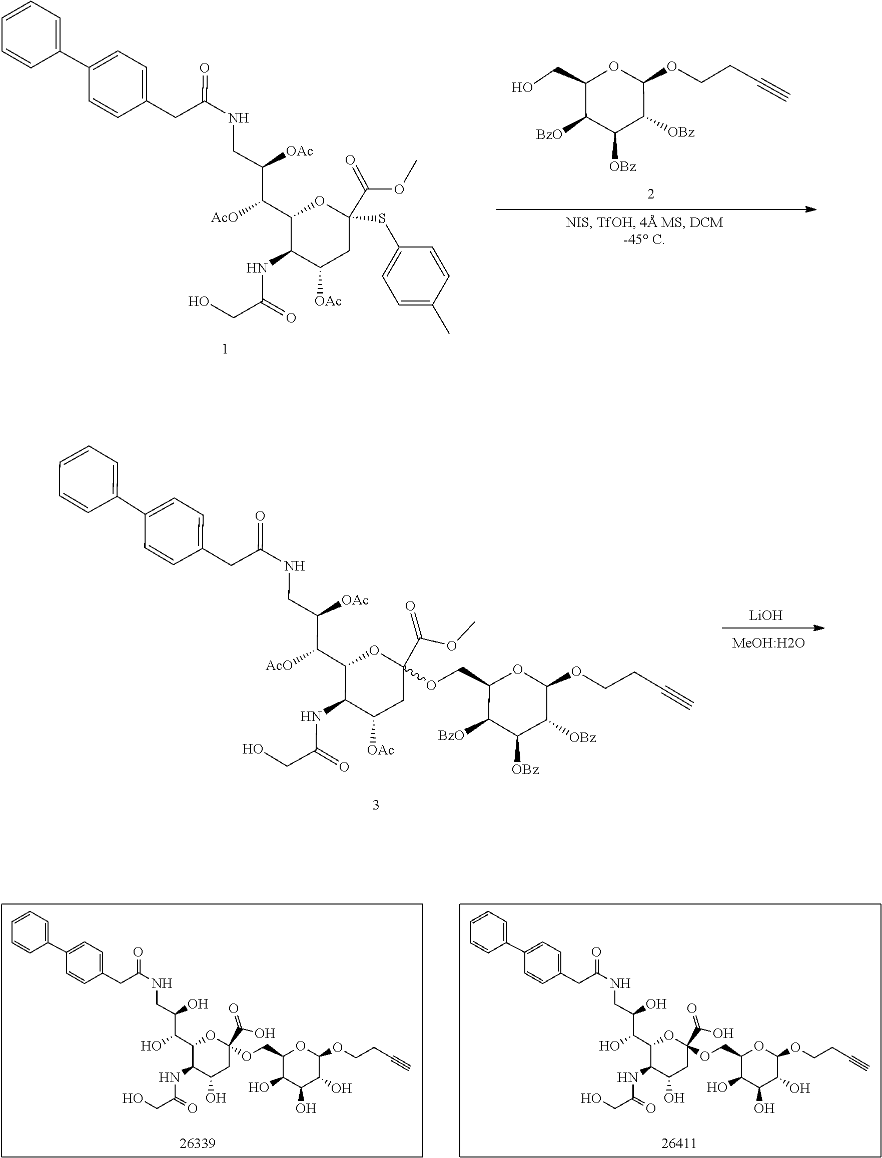

### Synthesis of (2R,3S,4S,5R,6R)-2-((((2R,4S,5R,6R)-6-((1R,2R)-3-(2-([1,1′-biphenyl]-4-yl)acetamido)-1,2-diacetoxypropyl)-4-acetoxy-5-(2-hydroxyacetamido)-2-(methoxycarbonyl)tetrahydro-2H-pyran-2-yl)oxy)methyl)-6-(but-3-yn-1-yloxy)tetrahydro-2H-pyran-3,4,5-triyl tribenzoate (3)

To a stirred solution of (1R,2R)-3-(2-([1,1′-biphenyl]-4-yl)acetamido)-1-((2R,3R,4S,6S)-4-acetoxy-3-(2-acetoxyacetamido)-6-(methoxycarbonyl)-6-(p-tolylthio)tetrahydro-2H-pyran-2-yl)propane-1,2-diyl diacetate (1, 1.50 g, 1.86 mmol) and (2R,3R,4S,5S,6R)-2-(but-3-yn-1-yloxy)-6-(hydroxymethyl)tetrahydro-2H-pyran-3,4,5-triyl tribenzoate (2, 2.53 g, 4.65 mmol) in dry dichloromethane (30 mL) was added 4 Å Molecular Sieves (1.50 g, 100% w/w). The resulting reaction mixture was stirred under nitrogen atmosphere for 12 h. After the reaction mixture was cooled to −45° C., 1-iodopyrrolidine-2,5-dione (1.05 g, 4.65 mmol) was added followed by the dropwise addition of trifluoromethanesulfonic acid (0.164 mL, 1.86 mmol). The reaction mixture was stirred at −45° C. for 30 min. After completion, the reaction mixture was quenched with trimethylamine up to neutral pH, filtered through celite, diluted with dichloromethane, and washed with aqueous sodium bicarbonate solution followed by DM water. The organic layer was concentrated to obtain a crude residue which was then purified via flash column chromatography (5-7.5% methanol in ethyl acetate). The desired fractions were concentrated and dried to afford (2R,3S,4S,5R,6R)-2-((((2R,4S,5R,6R)-6-((1R,2R)-3-(2-([1,1′-biphenyl]-4-yl)acetamido)-1,2-diacetoxypropyl)-4-acetoxy-5-(2-acetoxyacetamido)-2-(methoxycarbonyl)tetrahydro-2H-pyran-2-yl)oxy)methyl)-6-(but-3-yn-1-yloxy)tetrahydro-2H-pyran-3,4,5-triyl tribenzoate (3) as a white solid as an inseparable anomeric mixture. Yield: 1.40 g, 63.54%; LCMS (ESI) m/z 1186.20 [M+1]⁺.

### Synthesis of (2R,4S,5R,6R)-6-((1R,2R)-3-(2-([1,1′-biphenyl]-4-yl)acetamido)-1,2-dihydroxypropyl)-4-acetoxy-2-(((2R,3R,4S,5R,6R)-6-(but-3-yn-1-yloxy)-3,4,5-trihydroxytetrahydro-2H-pyran-2-yl)methoxy)-5-(2-hydroxyacetamido)tetrahydro-2H-pyran-2-carboxylic acid (Cpd. No. 26339) and (2S,4S,5R,6R)-6-((1R,2R)-3-(2-([1,1′-biphenyl]-4-yl)acetamido)-1,2-dihydroxypropyl)-4-acetoxy-2-(((2R,3R,4S,5R,6R)-6-(but-3-yn-1-yloxy)-3,4,5-trihydroxytetrahydro-2H-pyran-2-yl)methoxy)-5-(2-hydroxyacetamido)tetrahydro-2H-pyran-2-carboxylic acid (Cpd. No. 26411)

To a stirred solution of (2R,3S,4S,5R,6R)-2-((((2R,4S,5R,6R)-6-((1R,2R)-3-(2-([1,1′-biphenyl]-4-yl)acetamido)-1,2-diacetoxypropyl)-4-acetoxy-5-(2-acetoxyacetamido)-2-(methoxycarbonyl)tetrahydro-2H-pyran-2-yl)oxy)methyl)-6-(but-3-yn-1-yloxy)tetrahydro-2H-pyran-3,4,5-triyl tribenzoate (3, 1.40 g, 1.18 mmol) in methanol (5.0 mL) was added a solution of lithium hydroxide (0.071 g, 2.95 mmol) in water (1.0 mL) at 0° C. The reaction mixture was stirred at room temperature for 4 h. After completion, Dowex-Hydrogen form was added up to pH 6 and the reaction mass was filtered. The filtrate was concentrated and dried to obtain a crude residue which was diluted with acetonitrile and purified via preparatory HPLC (17-35% acetonitrile in water with 0.1% TFA). Fractions containing the desired products were separately combined and lyophilized to dryness to afford (2R,4S,5R,6R)-6-((1R,2R)-3-(2-([1,1′-biphenyl]-4-yl)acetamido)-1,2-dihydroxypropyl)-4-acetoxy-2-(((2R,3R,4S,5R,6R)-6-(but-3-yn-1-yloxy)-3,4,5-trihydroxytetrahydro-2H-pyran-2-yl)methoxy)-5-(2-hydroxyacetamido)tetrahydro-2H-pyran-2-carboxylic acid (Cpd. No. 26339) as the TFA salt as a white solid. Yield: 0.12 g; 13.11%; LCMS (ESI) m/z 733.18 [M+1]⁺; ¹H NMR (400 MHz, methanol-d₄) δ 7.99-7.97 (m, 1H), 7.88 (d, J=7.2 Hz, 1H), 7.61-7.57 (m, 4H), 7.43-7.37 (m, 4H), 7.31 (t, J=7.6 Hz, 1H), 4.25 (d, J=7.2 Hz, 1H), 4.01 (s, 2H), 3.94-3.88 (m, 3H), 3.86-3.82 (m, 3H), 3.79-3.63 (m, 5H), 3.60 (s, 2H), 3.52-3.44 (m, 2H), 3.38 (d, J=8.4 Hz, 1H), 2.70 (dd, J=13.2 & 3.6 Hz, 1H), 2.49 (dt, J=7.6 & 2.8 2H), 2.25 (t, J=2.8 Hz, 1H), 1.75 (t, J=13.2 Hz, 1H); (2S,4S,5R,6R)-6-((1R,2R)-3-(2-([1,1′-biphenyl]-4-yl)acetamido)-1,2-dihydroxypropyl)-4-acetoxy-2-(((2R,3R,4S,5R,6R)-6-(but-3-yn-1-yloxy)-3,4,5-trihydroxytetrahydro-2H-pyran-2-yl)methoxy)-5-(2-hydroxyacetamido)tetrahydro-2H-pyran-2-carboxylic acid (Cpd. No. 26411) as the TFA salt as a white solid. Yield: 0.230 g, 25.13%; LCMS (ESI) m/z 733.24 [M+1]⁺; ¹H NMR (400 MHz, methanol-d₄) δ 8.06-8.04 (m, 1H), 7.89 (d, J=9.2 Hz, 1H), 7.61-7.57 (m, 4H), 7.43-7.36 (m, 4H), 7.31 (t, J=7.6 Hz, 1H), 4.26 (d, J=7.2 Hz, 1H), 4.16-4.13 (m, 1H), 4.04-4.02 (m, 3H), 3.94-3.83 (m, 5H), 3.71-3.64 (m, 3H), 3.60 (s, 2H), 3.53-3.44 (m, 3H), 3.39 (d, J=9.2 H z, 1H), 2.50 (dt, J=7.2 & 2.4 2 H), 2.40 (dd, J=12.8 & 4.4 H z, 1H), 2.25 (t, J=2.4 H z, 1H), 1.66 (t, J=12.8 H z, 1H).

### (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26373)

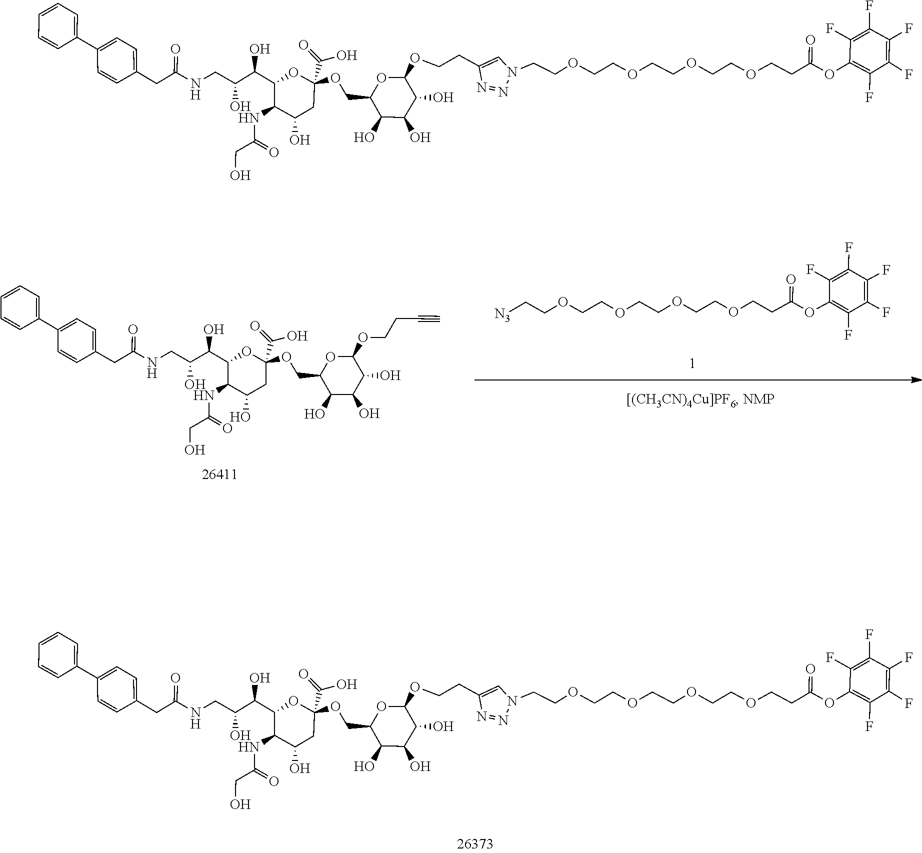

### Synthesis of (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26373)

To a solution of (2S,4S,5R,6R)-6-((1R,2R)-3-(2-([1,1′-biphenyl]-4-yl)acetamido)-1,2-dihydroxypropyl)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)tetrahydro-2H-pyran-2-carboxylic acid (26411, 1.00 eq, 19.5 mg, 0.0266 mmol) in NMP (0.3 mL) in a 1 dram vial with a stirbar was added a solution of perfluorophenyl 1-azido-3,6,9,12-tetraoxapentadecan-15-oate (1, 1.10 eq, 13.4 mg, 0.0293 mmol) in NMP (0.3 mL) followed by tetrakis(acetonitrile)copper(I) hexafluorophosphate (2.50 eq, 24.8 mg, 0.0665 mmol). The resulting clear green solution was capped and stirred at room temperature for 10 min. The reaction was diluted with acetic acid, filtered, and purified via preparatory HPLC (20-100% acetonitrile in water with 0.1% TFA). Fractions containing the desired product were combined and lyophilized to dryness to afford (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26373) as a white solid. Yield: 19.4 mg, 61%; LCMS m/z 1190.8 [M+1]⁺; ¹H NMR (300 MHz, DMSO with D₂O) δ 7.87 (s, 1H), 7.75 (d, J=8.8 Hz, 1H), 7.64-7.52 (m, 4H), 7.47-7.39 (m, 2H), 7.36-7.28 (m, 3H), 4.42 (t, J=5.0 Hz, 2H), 4.20-4.11 (m, 1H), 4.07-3.58 (m, 9H), 3.57-3.38 (m, 16H), 3.36-2.81 (m, 14H), 2.19-2.08 (m, 1H), 1.48 (t, J=12.1 Hz, 1H).

### (2R,4S,5R,6R)-5-acetamido-2-(((2R,3R,4S,5R,6R)-6-(but-3-yn-1-yloxy)-3,4,5-trihydroxytetrahydro-2H-pyran-2-yl)methoxy)-6-((1R,2R)-1,2-dihydroxy-3-(3-phenoxybenzamido)propyl)-4-hydroxytetrahydro-2H-pyran-2-carboxylic acid (Cpd. No. 26409) and (2S,4S,5R,6R)-5-acetamido-2-(((2R,3R,4S,5R,6R)-6-(but-3-yn-1-yloxy)-3,4,5-trihydroxytetrahydro-2H-pyran-2-yl)methoxy)-6-((1R,2R)-1,2-dihydroxy-3-(3-phenoxybenzamido)propyl)-4-hydroxytetrahydro-2H-pyran-2-carboxylic acid (Cpd. No. 26410)

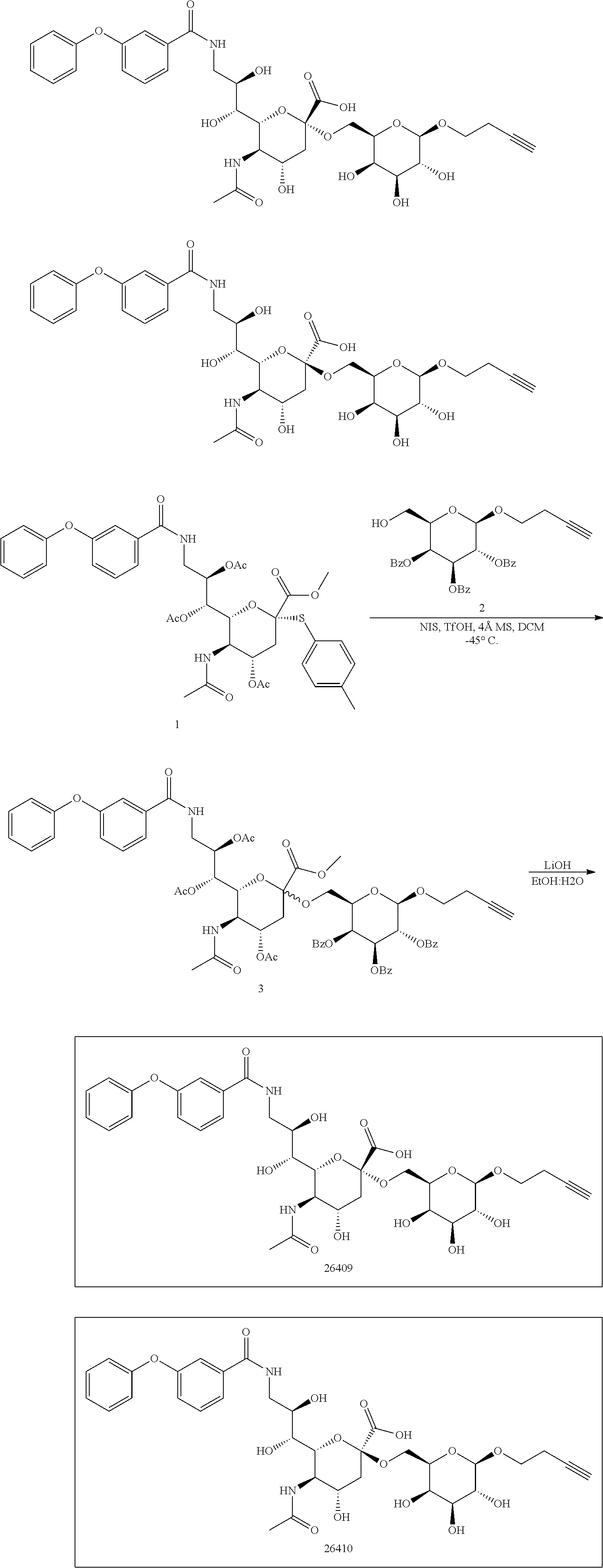

### Synthesis of (2R,3S,4S,5R,6R)-2-((((2R,4S,5R,6R)-5-acetamido-4-acetoxy-6-((1R,2R)-1,2-diacetoxy-3-(3-phenoxybenzamido)propyl)-2-(methoxycarbonyl)tetrahydro-2H-pyran-2-yl)oxy)methyl)-6-(but-3-yn-1-yloxy)tetrahydro-2H-pyran-3,4,5-triyl tribenzoate (3)

To a stirred solution of (1R,2R)-1-((2R,3R,4S,6S)-3-acetamido-4-acetoxy-6-(methoxycarbonyl)-6-(p-tolylthio)tetrahydro-2H-pyran-2-yl)-3-(3-phenoxybenzamido)propane-1,2-diyl diacetate (1, 0.50 g, 0.665 mmol) and (2R,3R,4S,5S,6R)-2-(but-3-yn-1-yloxy)-6-(hydroxymethyl)tetrahydro-2H-pyran-3,4,5-triyl tribenzoate (2, 0.543 g, 0.998 mmol) in dry dichloromethane (20 mL) was added 4 Å Molecular Sieves (0.50 g, 100% w/w). The resulting reaction mixture was stirred under nitrogen atmosphere for 12 h. Then, 1-iodopyrrolidine-2,5-dione (0.288 g, 1.28 mmol) and trifluoromethanesulfonic acid (0.047 mL, 0.533 mmol) were added at −45° C. The resulting reaction mixture was stirred at −45° C. for 30 min. After completion, the reaction mixture was quenched with trimethylamine up to neutral pH, filtered through celite, diluted with dichloromethane, and washed with aqueous sodium bicarbonate solution followed by DM water. The organic layer was concentrated to obtain a crude residue which was then purified via flash column chromatography (5-7.5% methanol in ethyl acetate). The desired fractions were concentrated and dried to afford (2R,3S,4S,5R,6R)-2-((((2R,4S,5R,6R)-5-acetamido-4-acetoxy-6-((1R,2R)-1,2-diacetoxy-3-(3-phenoxybenzamido)propyl)-2-(methoxycarbonyl)tetrahydro-2H-pyran-2-yl)oxy)methyl)-6-(but-3-yn-1-yloxy)tetrahydro-2H-pyran-3,4,5-triyl tribenzoate (3) as a yellow amorphous solid as an anomeric mixture. Yield: 0.60 g, 76.93%; LCMS (ESI) m/z 1172.17 [M+1]⁺.

### Synthesis of (2R,4S,5R,6R)-5-acetamido-2-(((2R,3R,4S,5R,6R)-6-(but-3-yn-1-yloxy)-3,4,5-trihydroxytetrahydro-2H-pyran-2-yl)methoxy)-6-((1R,2R)-1,2-dihydroxy-3-(3-phenoxybenzamido)propyl)-4-hydroxytetrahydro-2H-pyran-2-carboxylic acid (Cpd. No. 26409) and (2S,4S,5R,6R)-5-acetamido-2-(((2R,3R,4S,5R,6R)-6-(but-3-yn-1-yloxy)-3,4,5-trihydroxytetrahydro-2H-pyran-2-yl)methoxy)-6-((1R,2R)-1,2-dihydroxy-3-(3-phenoxybenzamido)propyl)-4-hydroxytetrahydro-2H-pyran-2-carboxylic acid (Cpd. No. 26410)

To a stirred solution of (2R,3S,4S,5R,6R)-2-((((2R,4S,5R,6R)-5-acetamido-4-acetoxy-6-((1R,2R)-1,2-diacetoxy-3-(3-phenoxybenzamido)propyl)-2-(methoxycarbonyl)tetrahydro-2H-pyran-2-yl)oxy)methyl)-6-(but-3-yn-1-yloxy)tetrahydro-2H-pyran-3,4,5-triyl tribenzoate (3, 0.60 g, 0.512 mmol) in ethanol (5.0 mL) at 0° C. was added lithium hydroxide (0.129 g, 3.07 mmol). The reaction mixture was stirred at room temperature for 4 h. After completion, Dowex-Hydrogen form was added up to pH 6 and the reaction mass was filtered. The filtrate was concentrated and dried to obtain a crude residue which was then diluted with acetonitrile and purified via preparatory HPLC (15-35% acetonitrile in water with 0.1% TFA). Fractions containing the desired products were separately combined and lyophilized to dryness to afford (2R,4S,5R,6R)-5-acetamido-2-(((2R,3R,4S,5R,6R)-6-(but-3-yn-1-yloxy)-3,4,5-trihydroxytetrahydro-2H-pyran-2-yl)methoxy)-6-((1R,2R)-1,2-dihydroxy-3-(3-phenoxybenzamido)propyl)-4-hydroxytetrahydro-2H-pyran-2-carboxylic acid (Cpd. No. 26409) as the TFA salt as a white solid. Yield: 0.045 g, 12%; LCMS (ESI) m/z 719.57 [M+1]⁺; ¹H NMR (400 MHz, methanol-d₄) δ 8.28-8.26 (m, 1H), 8.15-8.12 (m, 1H), 7.58 (d, J=7.6 Hz, 1H), 7.48-7.35 (m, 4H), 7.15-7.11 (m, 2H), 7.01 (d, J=8.0 Hz, 2H), 4.21 (t, J=3.6 Hz, 1H), 4.04-4.00 (m, 2H), 3.91-3.77 (m, 2H), 3.72-3.57 (m, 6H), 3.49-3.45 (m, 3H), 3.39 (d, J=8.8 Hz, 1H), 2.83-2.80 (m, 1H), 2.45-2.41 (m, 2H), 2.21 (t, J=2.8 Hz, 1H), 1.97 (s, 3H), 1.61 (t, J=11.6 Hz, 1H); (2S,4S,5R,6R)-5-acetamido-2-(((2R,3R,4S,5R,6R)-6-(but-3-yn-1-yloxy)-3,4,5-trihydroxytetrahydro-2H-pyran-2-yl)methoxy)-6-((1R,2R)-1,2-dihydroxy-3-(3-phenoxybenzamido)propyl)-4-hydroxytetrahydro-2H-pyran-2-carboxylic acid (Cpd. No. 26410) as the TFA salt as a white solid. Yield: 0.045 g, 12%; LCMS (ESI) m/z 719.57 [M+1]⁺; ¹H NMR (400 MHz, methanol-d₄) δ 7.59 (d, J=7.2 Hz, 1H), 7.49-7.35 (m, 4H), 7.15-7.13 (m, 2H), 7.00 (d, J=7.6 Hz, 2H), 4.25 (t, J=6.8 Hz, 1H), 3.99-3.85 (m, 6H), 3.81-3.63 (m, 4H), 3.54-3.44 (m, 4H), 3.36 (d, J=8.8 Hz, 1H), 2.49-2.45 (m, 2H), 2.42-2.37 (m, 1H), 2.23 (t, J=2.8 Hz, 1H), 1.95 (s, 3H), 1.60 (t, J=12.4 Hz, 1H).

### (2S,2′S,4S,4′S,5R,5′R,6R,6′R)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26412)

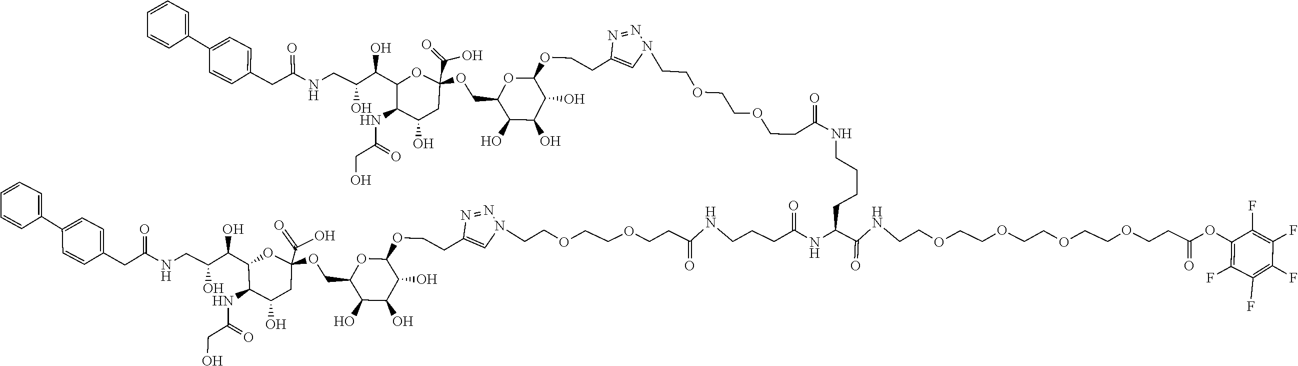

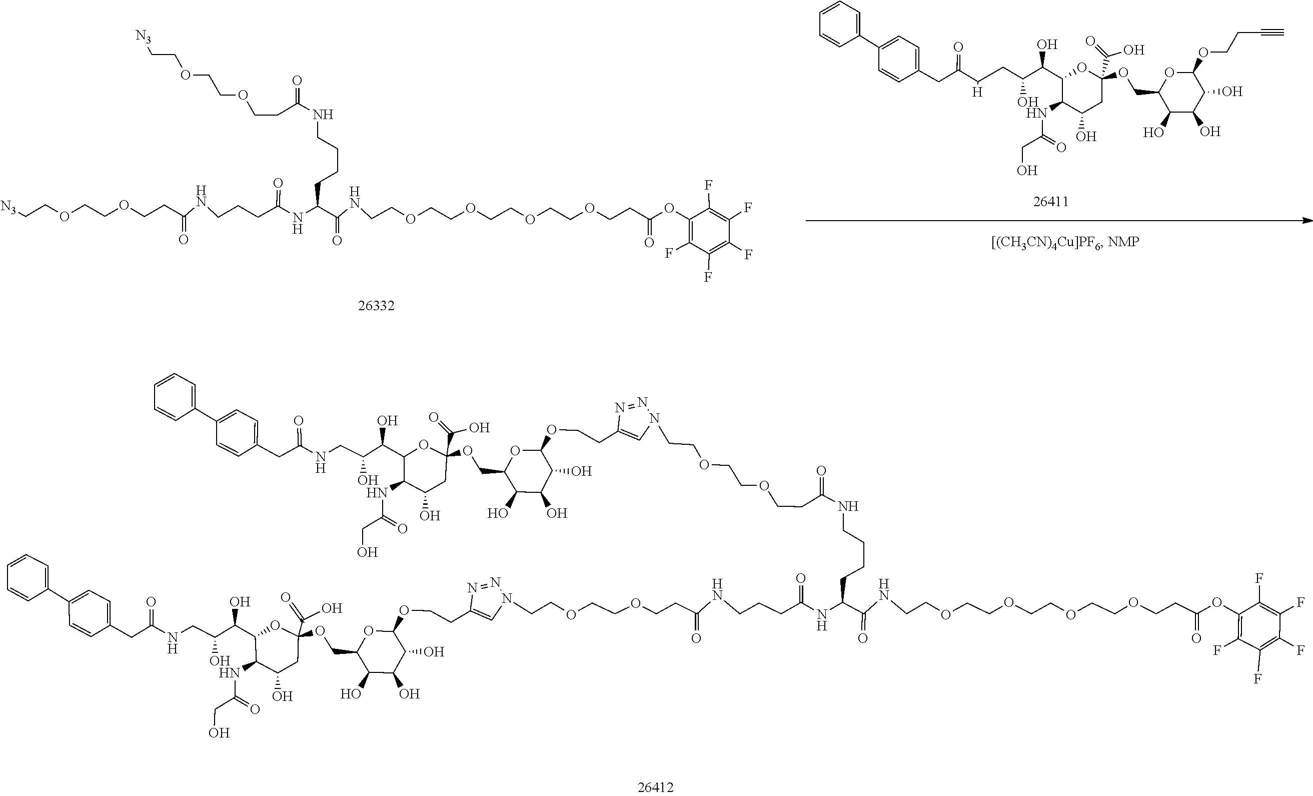

### Synthesis of (2S,2′S,4S,4′S,5R,5′R,6R,6′R)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1, 2, 3-triazole-1,4-diyl))bis(ethane-2,1-diyl))bis(oxy))bis(3,4,5-trihydroxytetrahydro-2H-pyran-6,2-diyl))bis(methylene))bis(oxy))bis(6-((1R,2R)-3-(2-([1,1′-biphenyl]-4-yl) acetamido)-1,2-dihydroxypropyl)-4-hydroxy-5-(2-hydroxyacetamido)tetrahydro-2H-pyran-2-carboxylic acid) (Cpd. No. 26412)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 14.3 mg, 0.0141 mmol) and (2S,4S,5R,6R)-6-((1R,2R)-3-(2-([1,1′-biphenyl]-4-yl)acetamido)-1,2-dihydroxypropyl)-2-(((2R,3R,4S,5R,6R)-6-(but-3-yn-1-yloxy)-3,4,5-trihydroxytetrahydro-2H-pyran-2-yl) methoxy)-4-hydroxy-5-(2-hydroxyacetamido)tetrahydro-2H-pyran-2-carboxylic acid (26411, 2.10 eq, 21.7 mg, 0.0296 mmol) in NMP (0.6 mL) in a 1 dram vial with a stirbar was added tetrakis(acetonitrile)copper(I) hexafluorophosphate (5.00 eq, 26.3 mg, 0.0704 mmol). The resulting clear green solution was capped and stirred at room temperature for 10 min. The reaction was diluted with acetic acid, filtered, and purified via preparatory HPLC (20-80% acetonitrile in water with 0.1% TFA). Fractions containing the desired product were combined and lyophilized to dryness to afford (2S,2′S,4S,4′S,5R,5′R,6R,6′R)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26412) as a white solid. Yield: 22.8 mg, 65%; LCMS m/z 1240.7 [M/2+1]+; ¹H NMR (300 MHz, DMSO with D₂O) δ 7.86 (s, 2H), 7.75 (d, J=8.8 Hz, 2H), 7.63-7.51 (m, 8H), 7.42 (t, J=7.5 Hz, 4H), 7.35-7.27 (m, 6H), 4.46-4.34 (m, 4H), 4.19-3.58 (m, 19H), 3.57-2.79 (m, 66H), 2.30-2.19 (m, 4H), 2.18-2.03 (m, 4H), 1.64-1.39 (m, 6H), 1.37-1.07 (m, 4H).

### (2S,2′S,4S,4′S,5R,5′R,6R,6′R)-2,2′-(((((((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(5-acetamido-6-((1R,2R)-1,2-dihydroxy-3-(3-phenoxybenzamido)propyl)-4-hydroxytetrahydro-2H-pyran-2-carboxylic acid) (Cpd. No. 26414)

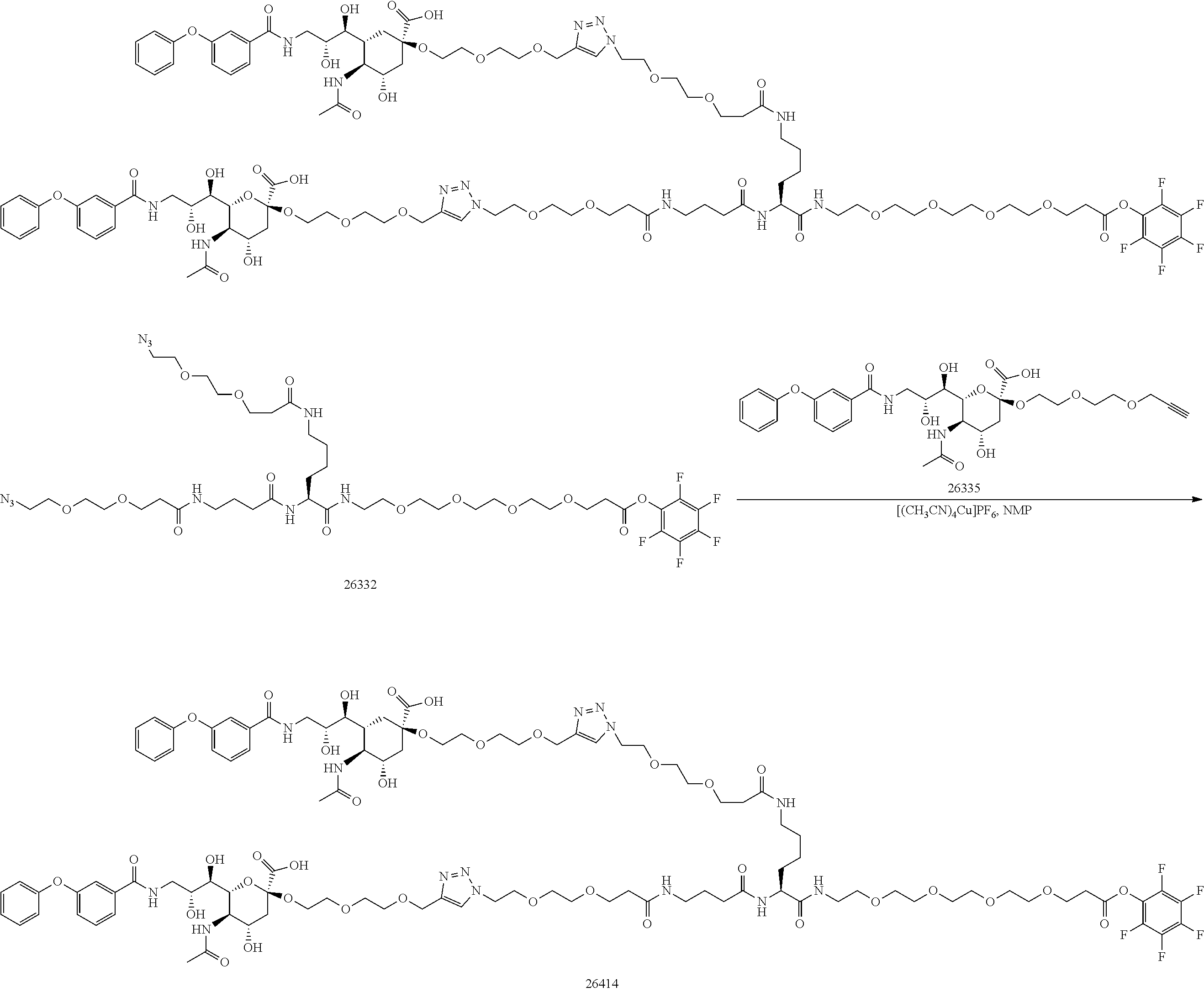

### Synthesis of (2S,2′S,4S,4′S,5R,5′R,6R,6′R)-2,2′-(((((((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(5-acetamido-6-((1R,2R)-1,2-dihydroxy-3-(3-phenoxybenzamido)propyl)-4-hydroxytetrahydro-2H-pyran-2-carboxylic acid) (Cpd. No. 26414)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 22.5 mg, 0.0222 mmol) and (2S,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26335, 2.10 eq, 29.4 mg, 0.0466 mmol) in NMP (0.6 mL) in a 1 dram vial with a stirbar was added tetrakis(acetonitrile)copper(I) hexafluorophosphate (5.00 eq, 41.3 mg, 0.111 mmol). The resulting clear light yellow solution was capped and stirred at room temperature for 10 min (slowly turned green). The reaction was diluted with acetic acid, filtered, and purified via preparatory HPLC (20-90% acetonitrile in water with 0.1% TFA). Fractions containing the desired product were combined and lyophilized to dryness to afford (2S,2′S,4S,4′S,5R,5′R,6R,6′R)-2,2′-(((((((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(5-acetamido-6-((1R,2R)-1,2-dihydroxy-3-(3-phenoxybenzamido)propyl)-4-hydroxytetrahydro-2H-pyran-2-carboxylic acid) (Cpd. No. 26414) as a white solid. Yield: 32.9 mg, 65%; LCMS m/z 1138.8 [M/2+1]+; ¹H NMR (300 MHz, DMSO with D₂O) δ 7.96 (s, 2H), 7.55 (d, J=7.7 Hz, 2H), 7.48-7.32 (m, 8H), 7.13 (t, J=7.4 Hz, 4H), 6.97 (d, J=8.0 Hz, 4H), 4.51-4.38 (m, 8H), 3.89-2.87 (m, 71H), 2.29-2.19 (m, 4H), 2.18-2.03 (m, 4H), 1.82 (s, 6H), 1.62-1.39 (m, 6H), 1.36-1.06 (m, 4H).

### (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26415)

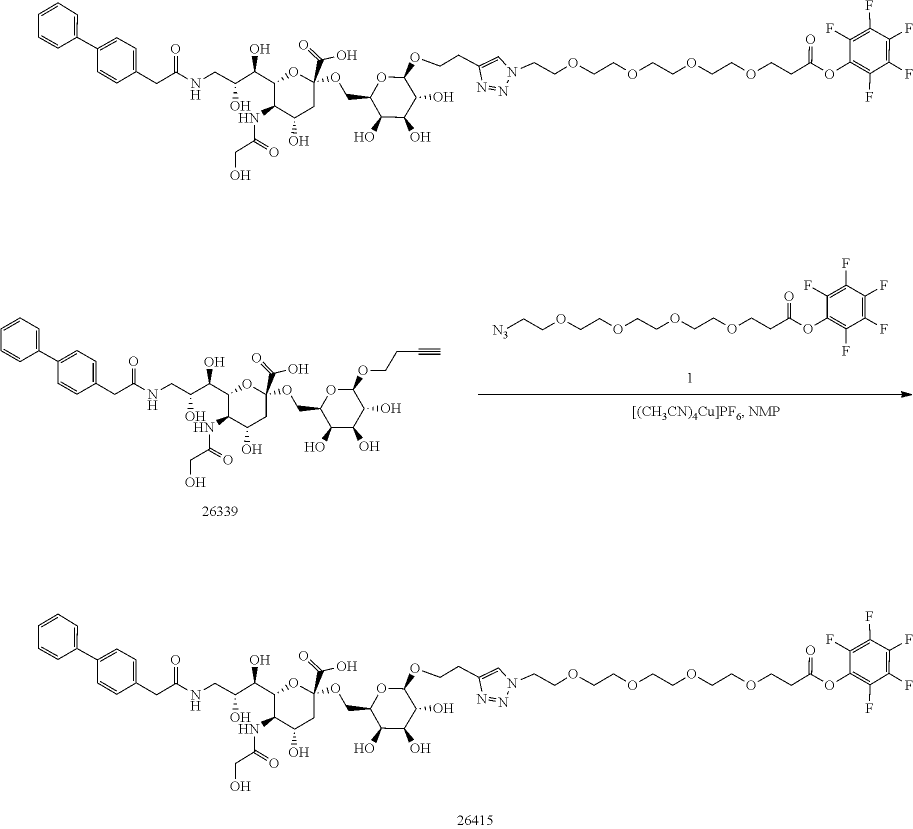

### Synthesis of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26415)

To a solution of (2R,4S,5R,6R)-6-((1R,2R)-3-(2-([1,1′-biphenyl]-4-yl)acetamido)-1,2-dihydroxypropyl)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)tetrahydro-2H-pyran-2-carboxylic acid (26339, 1.00 eq, 26.2 mg, 0.0358 mmol) in NMP (0.3 mL) in a 1 dram vial with a stirbar was added a solution of perfluorophenyl 1-azido-3,6,9,12-tetraoxapentadecan-15-oate (1, 1.10 eq, 18.0 mg, 0.0393 mmol) in NMP (0.3 mL) followed bytetrakis(acetonitrile)copper(I) hexafluorophosphate (2.50 eq, 33.3 mg, 0.0894 mmol). The resulting clear green solution was capped and stirred at room temperature for 10 min. The reaction was diluted with acetic acid, filtered, and purified via preparatory HPLC (20-70% acetonitrile in water with 0.1% TFA). Fractions containing the desired product were combined and lyophilized to dryness to afford (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26415) as a white solid. Yield: 26.5 mg, 62%; LCMS m/z 1190.7 [M+1]+; ¹H NMR (300 MHz, DMSO with D₂O) δ 7.83 (s, 1H), 7.79 (d, J=7.4 Hz, 1H), 7.62-7.50 (m, 4H), 7.42 (t, J=7.6 Hz, 2H), 7.34-7.26 (m, 3H), 4.39 (t, J=4.8 Hz, 2H), 4.15 (d, J=6.6 Hz, 1H), 3.96-2.79 (m, 38H), 2.47-2.40 (m, 1H), 1.55 (t, J=12.2 Hz, 1H).

### (2R,2′R,4S,4′S,5R,5′R,6R,6′R)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26416)

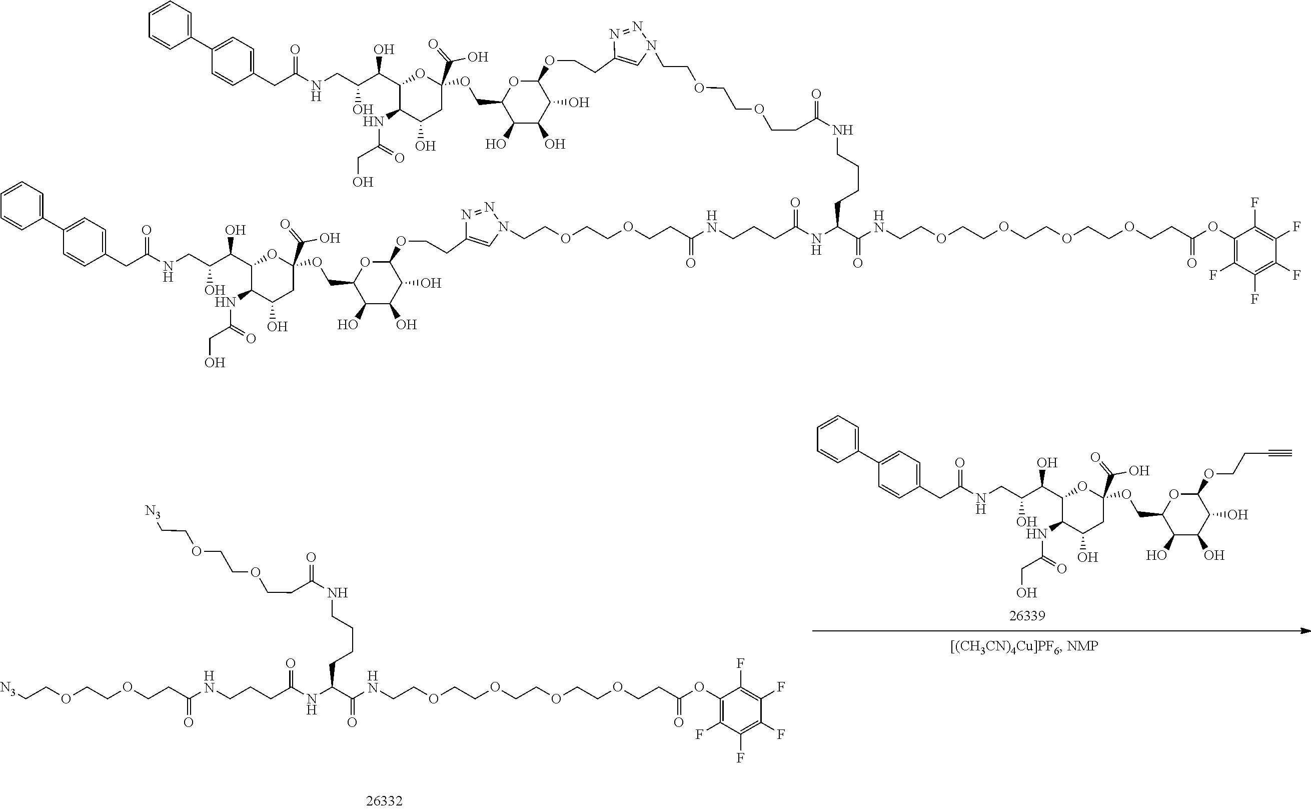

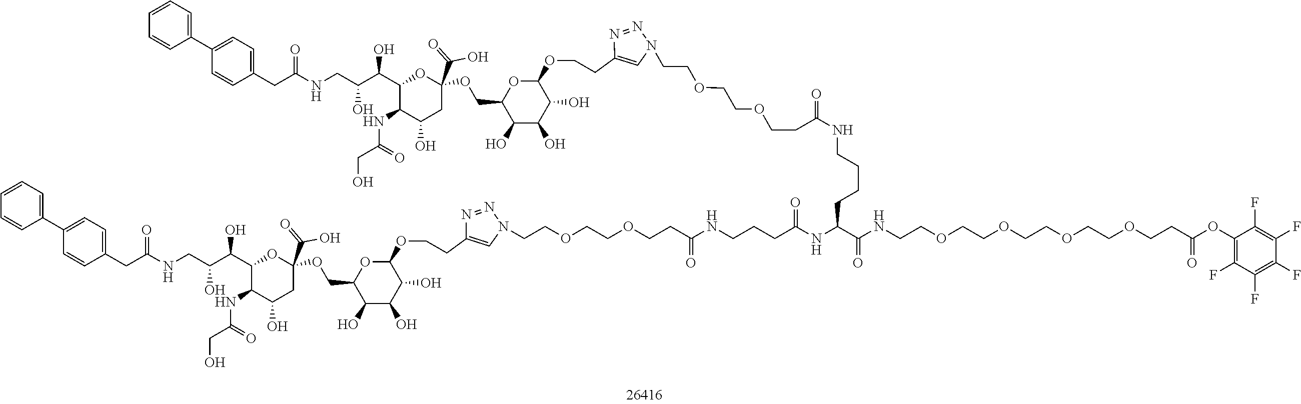 [SMILES: O=C(CCOCCOCCn1cc(CCO[C@@H]2O[C@H](CO[C@]3(C(=O)O)C[C@H](O)[C@@H](NC(=O)CO)[C@H]([C@H](O)[C@H](O)CNC(=O)Cc4ccc(-c5ccccc5)cc4)O3)[C@H](O)[C@H](O)[C@H]2O)nn1)NCCCC[C@H](NC(=O)CCCNC(=O)CCOCCOCCn1cc(CCO[C@@H]2O[C@H](CO[C@]3(C(=O)O)C[C@H](O)[C@@H](NC(=O)CO)[C@H]([C@H](O)[C@H](O)CNC(=O)Cc4ccc(-c5ccccc5)cc4)O3)[C@H](O)[C@H](O)[C@H]2O)nn1)C(=O)NCCOCCOCCOCCOCCC(=O)Oc1c(F)c(F)c(F)c(F)c1F]

### Synthesis of (2R,2′R,4S,4′S,5R,5′R,6R,6′R)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26416)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 20.7 mg, 0.0204 mmol) and (2R,4S,5R,6R)-6-((1R,2R)-3-(2-([1,1′-biphenyl]-4-yl)acetamido)-1,2-dihydroxypropyl)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)tetrahydro-2H-pyran-2-carboxylic acid (26339, 2.10 eq, 31.4 mg, 0.0428 mmol) in NMP (0.6 mL) in a 1 dram vial with a stirbar was added tetrakis(acetonitrile)copper(I) hexafluorophosphate (5.00 eq, 38.0 mg, 0.102 mmol). The resulting clear green solution was capped and stirred at room temperature for 10 min. The reaction was diluted with acetic acid, filtered, and purified via preparatory HPLC (20-65% acetonitrile in water with 0.1% TFA). Fractions containing the desired product were combined and lyophilized to dryness to afford (2R,2′R,4S,4′S,5R,5′R,6R,6′R)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26416) as a white solid. Yield: 27.2 mg, 54%; LCMS m/z 1240.8 [M/2+1]⁺; ¹H NMR (300 MHz, DMSO with D₂O) δ 7.84-7.75 (m, 4H), 7.61-7.49 (m, 8H), 7.41 (t, J=7.5 Hz, 4H), 7.34-7.25 (m, 6H), 4.42-4.32 (m, 4H), 3.94-2.76 (m, 85H), 2.47-2.39 (m, 2H), 2.30-2.17 (m, 4H), 2.08 (t, J=7.5 Hz, 2H), 1.64-1.37 (m, 6H), 1.35-1.07 (m, 4H).

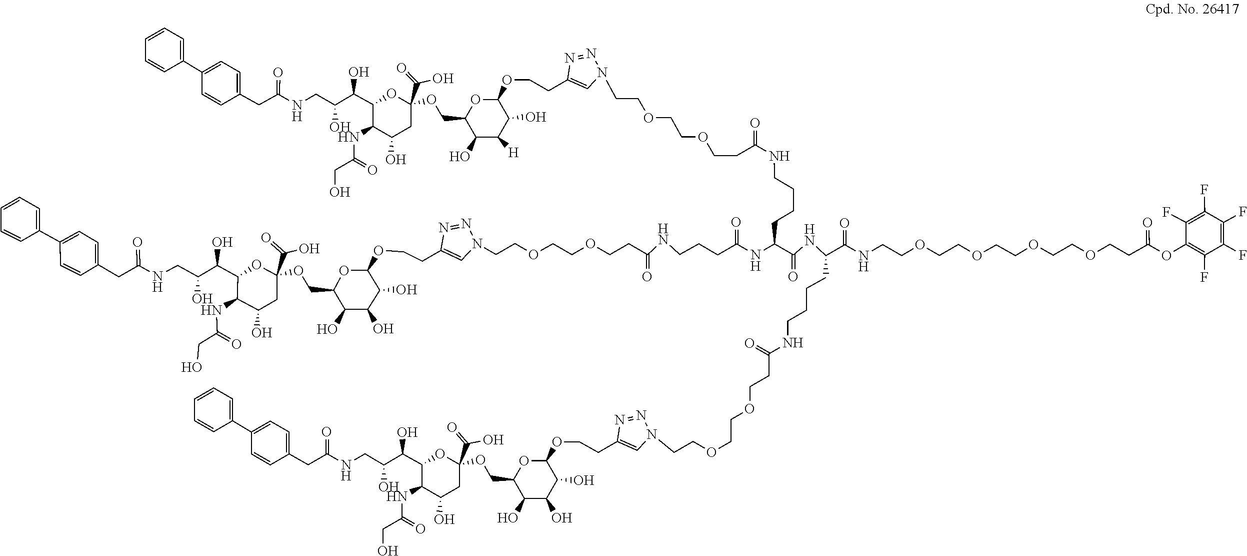

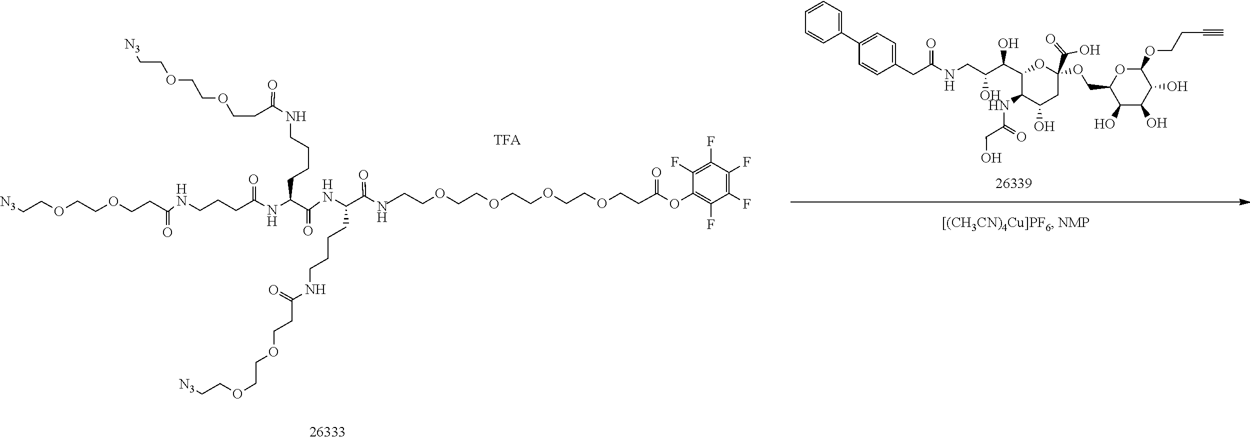 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCCOCCOCCOCCOCCC(=O)Oc1c(F)c(F)c(F)c(F)c1F ; C#CCCO[C@@H]1O[C@H](CO[C@]2(C(=O)O)C[C@H](O)[C@@H](NC(=O)CO)[C@H]([C@H](O)[C@H](O)CNC(=O)Cc3ccc(-c4ccccc4)cc3)O2)[C@H](O)[C@H](O)[C@H]1O]

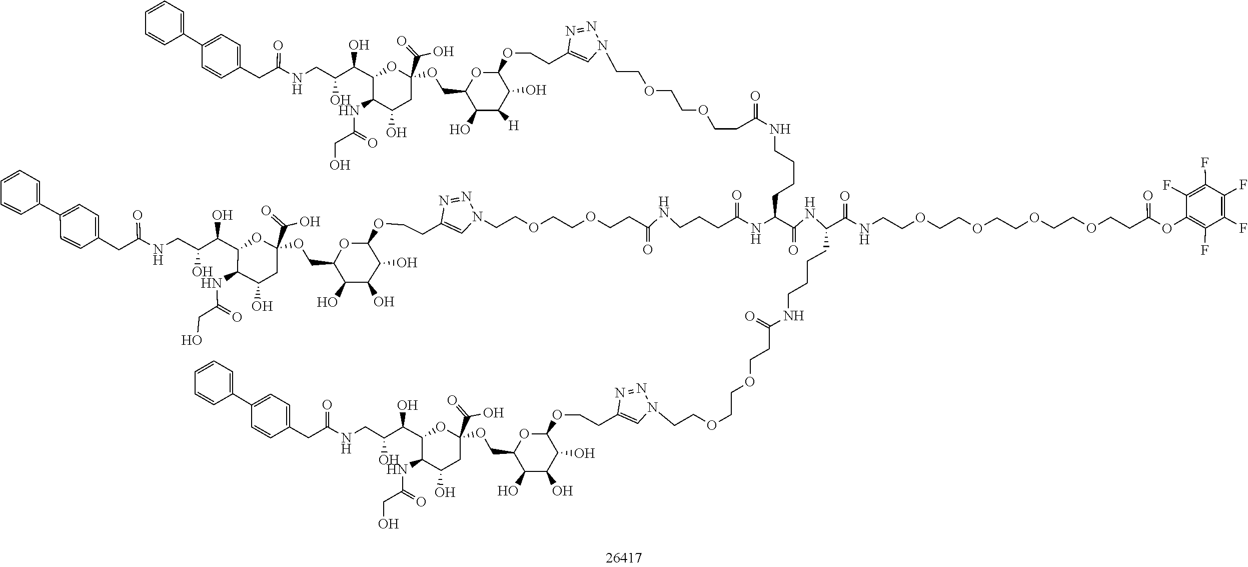

Synthesis of Cpd. No. 26417

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 19.1 mg, 0.0144 mmol) and (2R,4S,5R,6R)-6-((1R,2R)-3-(2-([1,1′-biphenyl]-4-yl)acetamido)-1,2-dihydroxypropyl)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)tetrahydro-2H-pyran-2-carboxylic acid (26339, 3.10 eq, 32.7 mg, 0.0446 mmol) in NMP (0.6 mL) in a 1 dram vial with a stirbar was added tetrakis(acetonitrile)copper(I) hexafluorophosphate (7.50 eq, 40.2 mg, 0.108 mmol). The resulting clear green solution was capped and stirred at room temperature for 10 min. The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford Cpd. No. 26417 as a white solid. Yield: 22.8 mg, 45%; LCMS m/z [M+1]⁺; ¹H NMR (300 MHz, DMSO with D₂O) δ 7.83-7.73 (m, 6H), 7.60-7.48 (m, 12H), 7.40 (t, J=7.5 Hz, 6H), 7.33-7.25 (m, 9H), 4.40-4.32 (m, 6H), 3.93-2.77 (m, 120H), 2.47-2.40 (m, 2H), 2.30-2.18 (m, 6H), 2.13-2.03 (m, 2H), 1.66-1.38 (m, 8H), 1.36-1.06 (m, 8H).

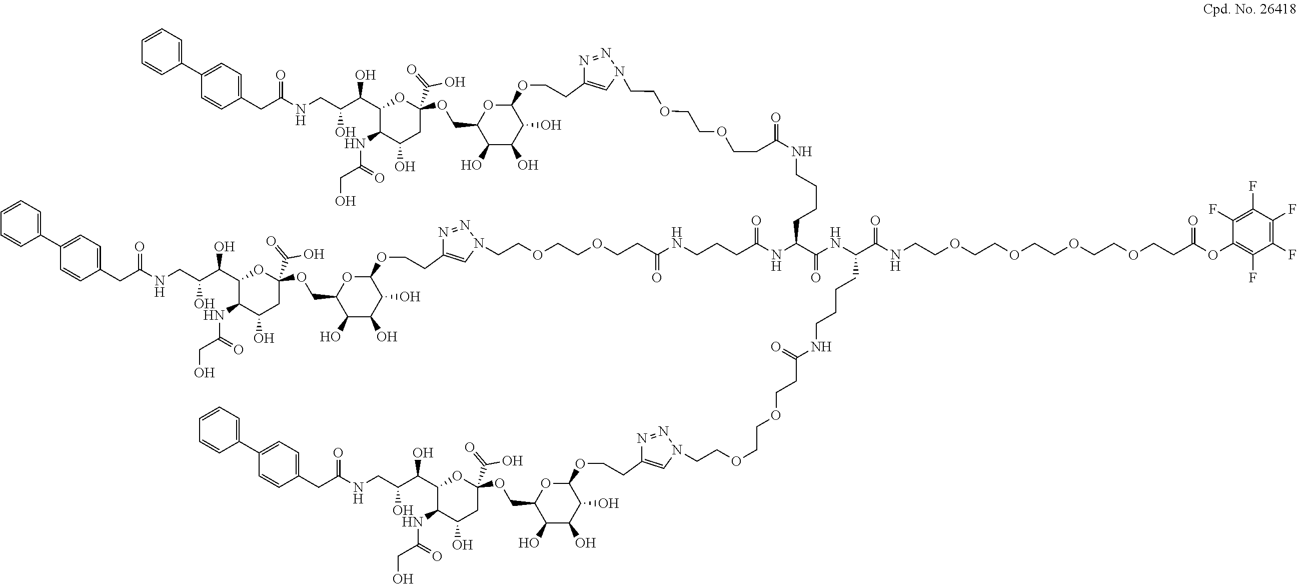

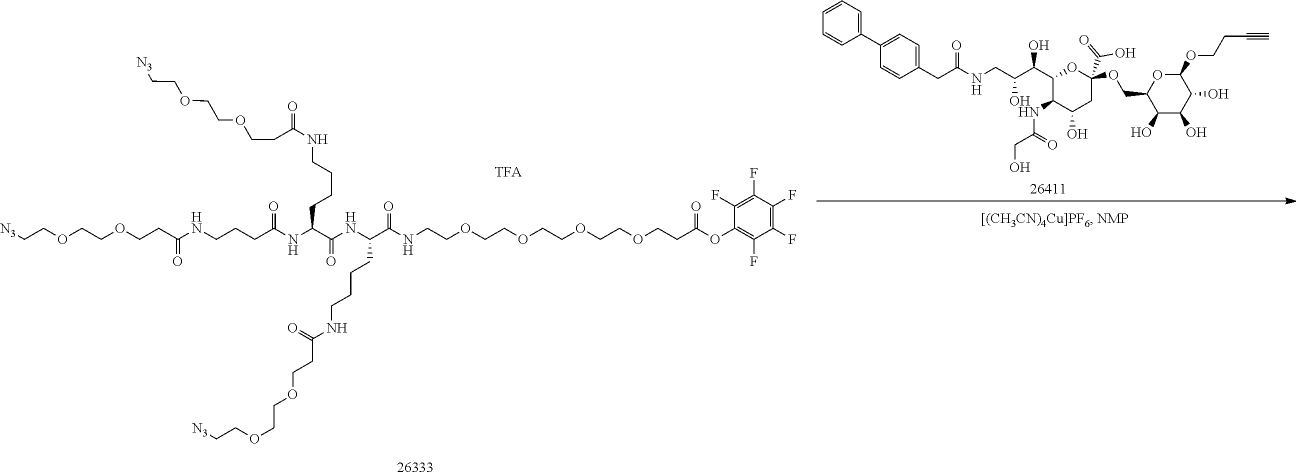 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F ; C#CCCO[C@@H]1O[C@H](CO[C@@]2(C(=O)O)C[C@H](O)[C@@H](NC(=O)CO)[C@H]([C@H](O)[C@H](O)CNC(=O)Cc3ccc(-c4ccccc4)cc3)O2)[C@H](O)[C@H](O)[C@H]1O]

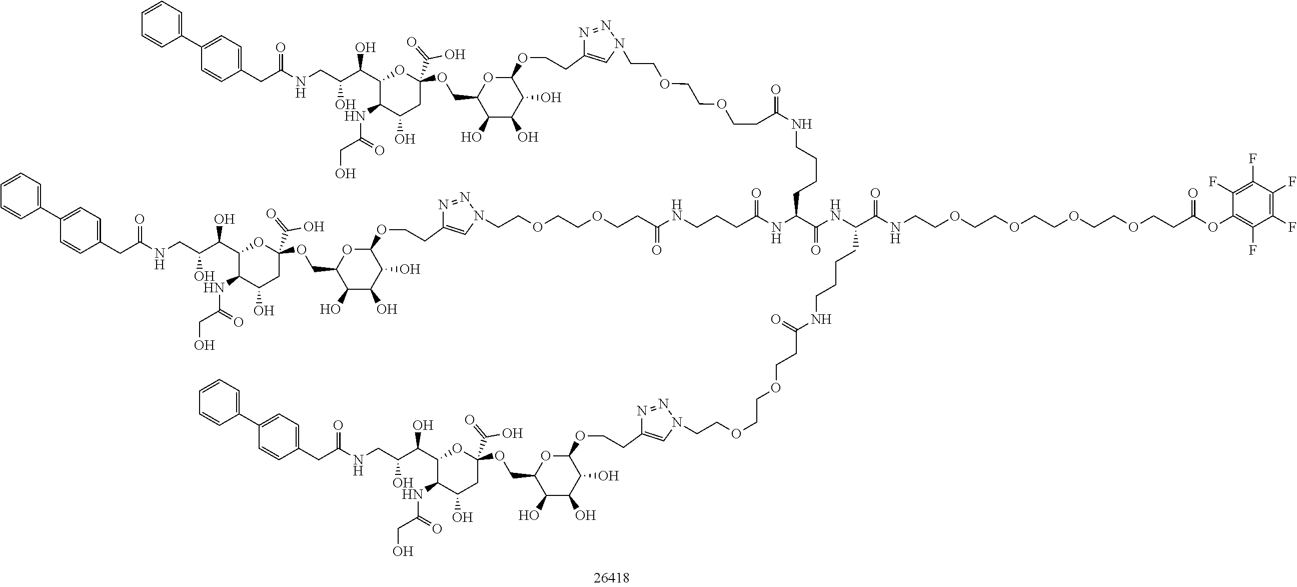

Synthesis of Cpd. No. 26418

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 18.3 mg, 0.0138 mmol) and (2S,4S,5R,6R)-6-1-yloxy)-3,4,5-trihydroxytetrahydro-2H-pyran-2-yl)methoxy)-4-hydroxy-5-(2-hydroxyacetamido)tetrahydro-2H-pyran-2-carboxylic acid (26411, 3.10 eq, 31.3 mg, 0.0427 mmol) in NMP (0.6 mL) in a 1 dram vial with a stirbar was added tetrakis(acetonitrile)copper(I) hexafluorophosphate (7.50 eq, 38.5 mg, 0.103 mmol). The resulting clear green solution was capped and stirred at room temperature for 10 min. The reaction was diluted with a mixture of 70% acetic acid in NMP, filtered, and purified via preparatory HPLC (15-65% acetonitrile in water with 0.1% TEA). Fractions containing the desired product were combined and lyophilized to dryness to afford Cpd. No. 26418 as a white solid. Yield: 32.7 mg, 67%; LCMS m/z 1763.7 [M/2+1]⁺; ¹H NMR (300 M Hz, DMSO with D₂O) δ 7.83 (s, 3H), 7.76 (d, J=8.9 Hz, 3H), 7.61-7.49 (m, 12H), 7.40 (t, J=7.5 H z, 6H), 7.34-7.26 (m, 9H), 4.43-4.34 (m, 6H), 4.02-2.80 (in, 120H), 2.30-2.04 (in, 10H), 1.64-1.40 (m, 8H), 1.35-1.08 (in, 8H).

### (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. No. 26463) and (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. No. 26464)

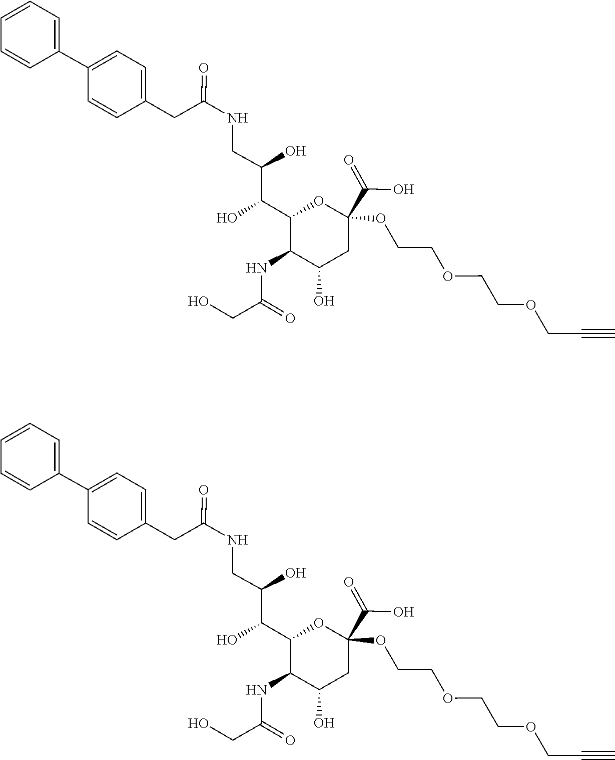 [SMILES: C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1 ; C#CCOCCOCCO[C@@]1(C(=O)O)C[C@H](O)[C@@H](NC(=O)CO)[C@H]([C@H](O)[C@H](O)CNC(=O)Cc2ccc(-c3ccccc3)cc2)O1]

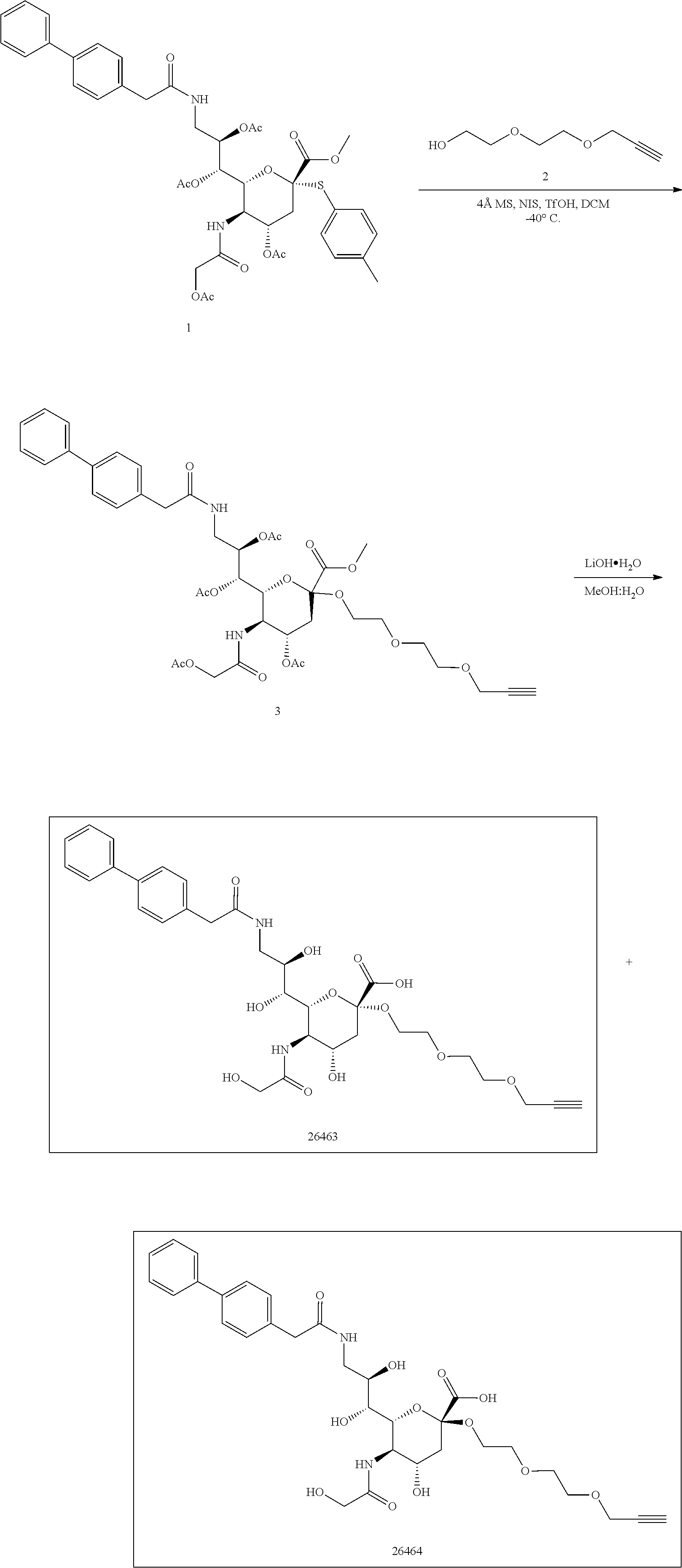

### Synthesis of (1R,2R)-3-(2-([1,1′-biphenyl]-4-yl)acetamido)-1-((2R,3R,4S)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3)

To a stirred solution of (1R,2R)-3-(2-([1,1′-biphenyl]-4-yl)acetamido)-1-((2R,3R,4S,6S)-4-acetoxy-3-(2-acetoxyacetamido)-6-(methoxycarbonyl)-6-(p-tolylthio)tetrahydro-2H-pyran-2-yl)propane-1,2-diyl diacetate (1, 1.0 g, 1.240 mmol) in anhydrous dichloromethane (20.0 mL) was added 2-(2-(prop-2-yn-1-yloxy)ethoxy)ethan-1-ol (2, 0.893 g, 6.20 mmol) and activated 4 Å powdered molecular sieves (1.0 g, 100% w/w). The resulting reaction solution was stirred at 15 h at room temperature under nitrogen. To the solution was added 1-iodopyrrolidine-2,5-dione (0.697 g, 3.10 mmol) and trifluoromethanesulfonic acid (0.109 mL, 1.240 mmol) at −40° C. The resulting reaction solution was stirred at −40° C. for 1 h. After completion, the reaction mixture was quenched with triethyl amine (0.5 mL) and warmed to room temperature. The reaction mixture was filtered through a sintered funnel and washed with dichloromethane. The filtrate was washed with saturated sodium bicarbonate (aq), dried over sodium sulfate, filtered, and concentrated under reduced pressure to obtain a crude residue. The residue was purified via column chromatography (60-80% ethyl acetate in hexanes) to afford (1R,2R)-3-(2-([1,1′-biphenyl]-4-yl)acetamido)-1-((2R,3R,4S)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3) as a white solid as an anomeric mixture. Yield: 0.80 g, 78.07%; LCMS (ESI) m/z 827.30 [M+1]⁺.

### Synthesis of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. No. 26463) and (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. No. 26464)

To a stirred solution of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3, 0.450 g, 0.544 mmol) in methanol (5.0 mL) was added a solution of Lithium hydroxide monohydrate (0.137 g, 3.27 mmol) in water (0.50 mL). The resulting reaction mixture was stirred at room temperature for 6 h. After completion, the reaction mixture was treated with Dowex 50, H+) up to pH ^{˜}6 and the suspension was filtered and washed with methanol. The filtrate was concentrated under reduced pressure to obtain a crude residue. The residue was purified via preparatory HPLC to afford (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. No. 26463) as a white solid. Yield: 0.316 g, 90%; LCMS (ESI) m/z 645.45 [M+1]⁺. ¹H NMR (400 MHz, Methanol-d₄) δ 7.87 (d, J=7.6 Hz, 1H), 7.61-7.56 (m, 4H), 7.43-7.37 (m, 4H), 7.31 (t, J=7.2 Hz, 1H), 4.17 (d, J=2.4 Hz, 2H), 3.90 (s, 2H), 3.89-3.81 (m, 4H), 3.74-3.59 (m, 11H), 3.39-3.25 (m, 2H), 2.82 (t, J=0.8 Hz, 1H), 2.71 (dd, J=8.8 & 4.0 Hz, 1H), 1.75 (t, J=12.4 Hz, 1H); (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. No. 26464) as a white solid. Yield: 0.192 g, 80.25%; LCMS (ESI) m/z 645.42 [M+1]⁺. ¹H NMR (400 MHz, Methanol-d₄) δ 7.82 (d, J=8.8 Hz, 1H), 7.61-7.57 (m, 4H), 7.44-7.36 (m, 4H), 7.31 (t, J=7.2 Hz, 1H), 4.17 (d, J=2.4 Hz, 2H), 4.15-4.119 (m, 1H), 4.03-3.96 (m, 3H), 3.94-3.86 (m, 1H), 3.86-3.78 (m, 2H), 3.36-3.62 (m, 2H) 3.59 (s, 2H), 3.51-3.46 (m, 1H), 3.42-3.35 (m, 2H), 2.84 (t, J=2.4 Hz, 1H), 2.38 (dd, J=12.8 & 3.8 Hz, 1H), 1.66 (t, J=11.6 Hz, 1H).

### (2S,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26465)

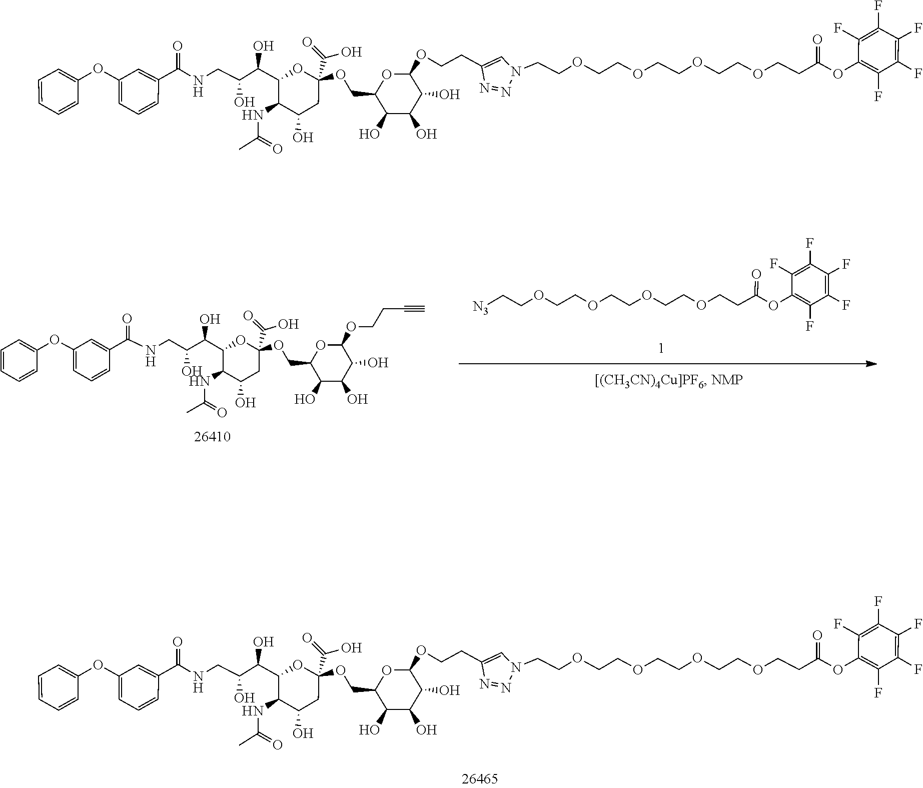

### Synthesis of (2S,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26465)

To a solution of (2S,4S,5R,6R)-5-acetamido-2-(((2R,3R,4S,5R,6R)-6-(but-3-yn-1-yloxy)-3,4,5-trihydroxytetrahydro-2H-pyran-2-yl)methoxy)-6-((1R,2R)-1,2-dihydroxy-3-(3-phenoxybenzamido)propyl)-4-hydroxytetrahydro-2H-pyran-2-carboxylic acid (26410, 1.00 eq, 25.8 mg, 0.0359 mmol) in NMP (0.3 mL) in a 1 dram vial with a stirbar was added a solution of perfluorophenyl 1-azido-3,6,9,12-tetraoxapentadecan-15-oate (1, (1.10 eq, 18.1 mg, 0.0395 mmol) in NMP (0.3 mL) followed by tetrakis(acetonitrile)copper(I) hexafluorophosphate (2.50 eq, 33.5 mg, 0.0898 mmol). The resulting clear green solution was capped and stirred at room temperature for 10 min. The reaction was diluted with acetic acid, filtered, and purified via preparatory HPLC (15-65% acetonitrile in water with 0.1% TFA). Fractions containing the desired product were combined and lyophilized to dryness to afford (2S,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Cpd. No. 26465) as a white solid. Yield: 23.5 mg, 56%; LCMS m/z 1176.8 [M+1]+; ¹H NMR (300 MHz, DMSO-d₆ with D₂O) δ 7.93 (d, J=7.5 Hz, 1H), 7.84 (s, 1H), 7.55 (d, J=7.7 Hz, 1H), 7.45 (t, J=7.9 Hz, 1H), 7.41-7.32 (m, 3H), 7.14 (t, J=7.1 Hz, 2H), 6.97 (d, J=8.0 Hz, 2H), 4.44-4.34 (m, 2H), 3.95-3.78 (m, 2H), 3.78-2.95 (m, 30H), 2.95-2.81 (m, 4H), 2.18-2.06 (m, 1H), 1.83 (s, 3H), 1.54-1.40 (m, 1H).

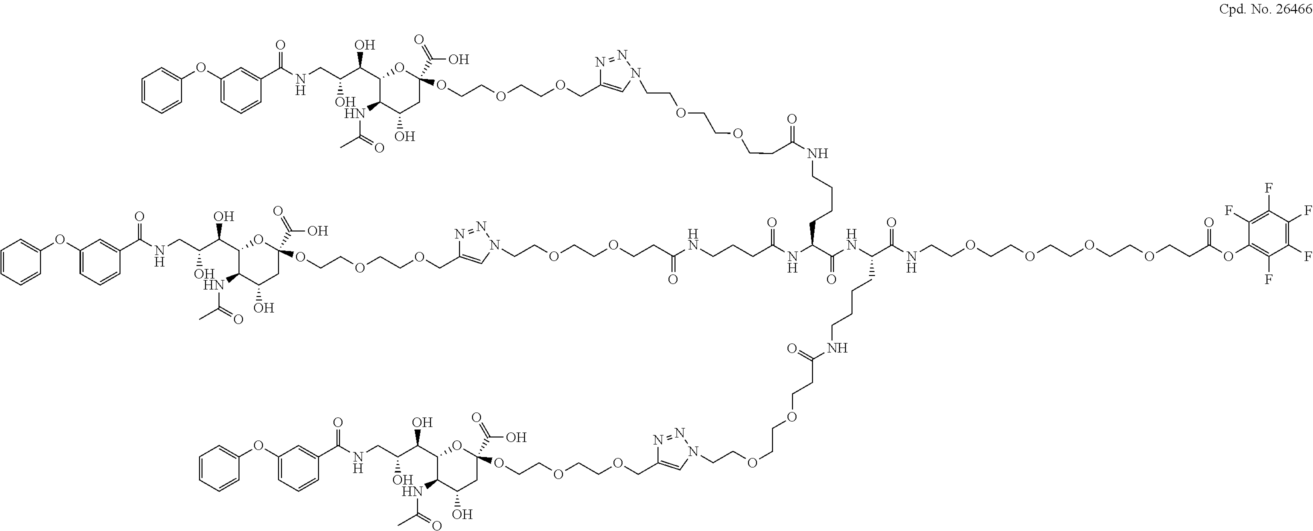 [SMILES: CC(=O)N[C@H]1[C@H]([C@H](O)[C@H](O)CNC(=O)c2cccc(Oc3ccccc3)c2)O[C@](OCCOCCOCc2cn(CCOCCOCCC(=O)NCCCC[C@H](NC(=O)CCCNC(=O)CCOCCOCCn3cc(COCCOCCO[C@@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)N[C@@H](CCCCNC(=O)CCOCCOCCn3cc(COCCOCCO[C@@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)NCCOCCOCCOCCOCCC(=O)Oc3c(F)c(F)c(F)c(F)c3F)nn2)(C(=O)O)C[C@@H]1O]

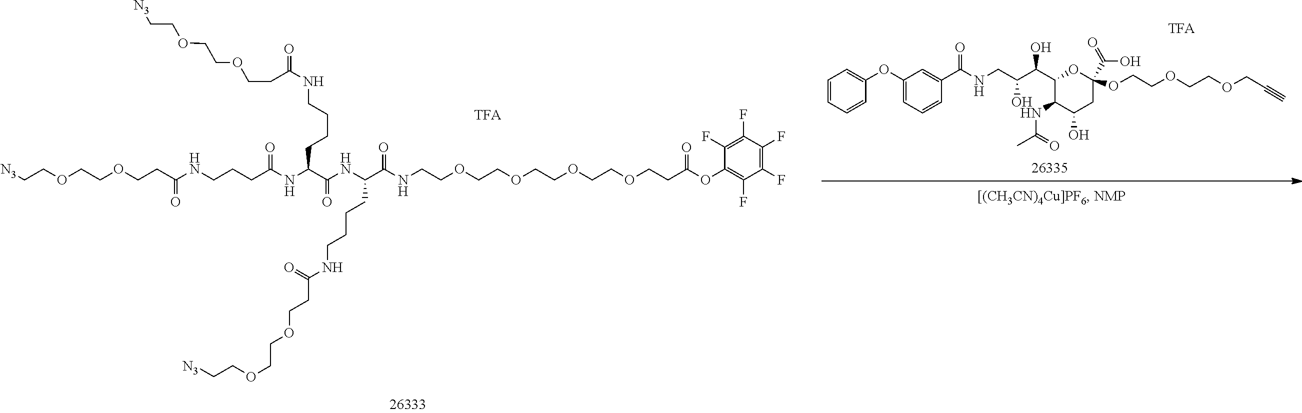 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F ; C#CCOCCOCCO[C@@]1(C(=O)O)C[C@H](O)[C@@H](NC(C)=O)[C@H]([C@H](O)[C@H](O)CNC(=O)c2cccc(Oc3ccccc3)c2)O1]

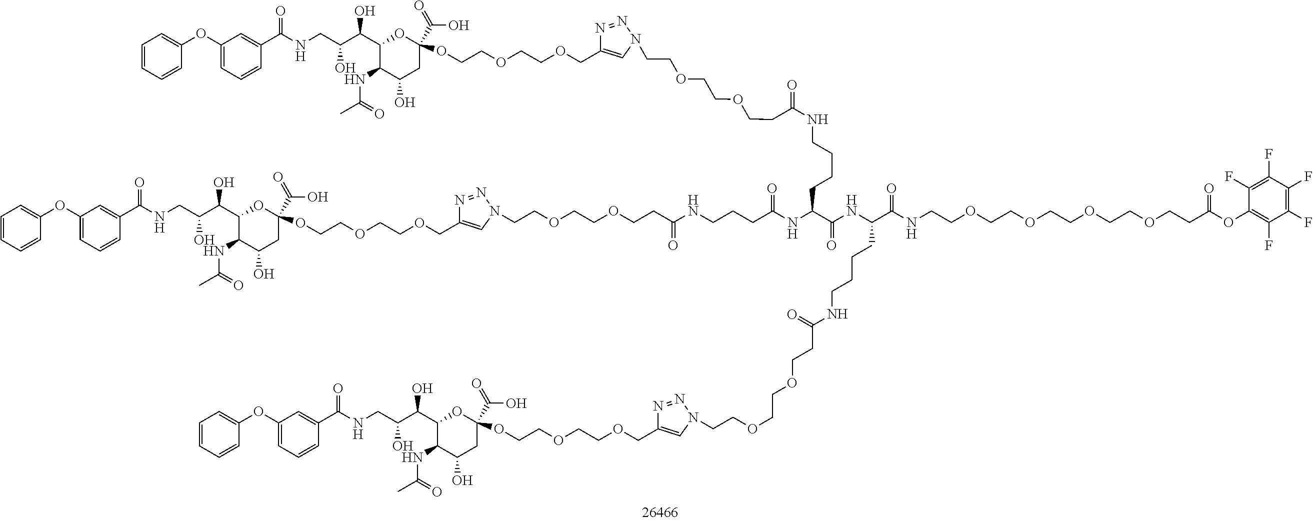 [SMILES: CC(=O)N[C@H]1[C@H]([C@H](O)[C@H](O)CNC(=O)c2cccc(Oc3ccccc3)c2)O[C@](OCCOCCOCc2cn(CCOCCOCCC(=O)NCCCC[C@H](NC(=O)CCCNC(=O)CCOCCOCCn3cc(COCCOCCO[C@@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)N[C@@H](CCCCNC(=O)CCOCCOCCn3cc(COCCOCCO[C@@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)NCCOCCOCCOCCOCCC(=O)Oc3c(F)c(F)c(F)c(F)c3F)nn2)(C(=O)O)C[C@@H]1O]

Synthesis of Cpd. No. 26466

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 23.1 mg, 0.0160 mmol) and (2R,4R,5S,6S)-5-acetamido-6-((1S,2S)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26335, 3.10 eq, 36.9 mg, 0.0495 mmol) in NMP (0.6 mL) in a 1 dram vial with a stirbar was added tetrakis(acetonitrile)copper(I) hexafluorophosphate (7.50 eq, 44.7 mg, 0.120 mmol). The resulting clear light yellow solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford Cpd. No. 26466 as a white solid. Yield: 35.4 mg, 69%; LCMS m/z 1610.7 [M/2+1]+; ¹H NMR (300 MHz, DMSO-d₆ with D₂O) δ 7.95 (s, 3H), 7.54 (d, J=7.8 Hz, 3H), 7.47-7.30 (m, 12H), 7.12 (t, J=7.4 Hz, 6H), 6.96 (d, J=8.0 Hz, 6H), 4.52-4.37 (m, 12H), 3.90-2.78 (m, 99H), 2.29-2.18 (m, 6H), 2.18-2.02 (m, 4H), 1.82 (s, 9H), 1.68-1.36 (m, 8H), 1.36-1.05 (m, 8H).

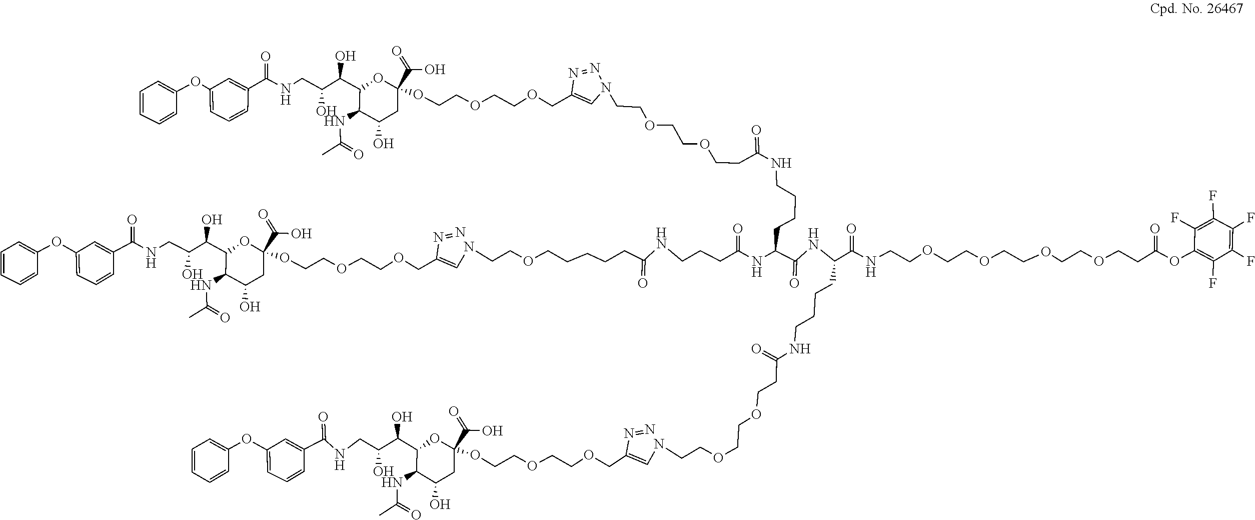

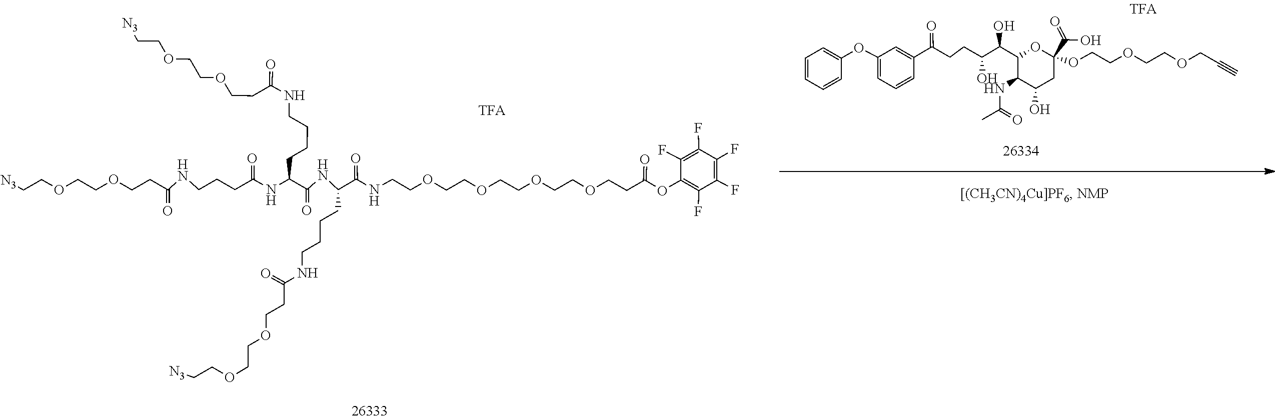

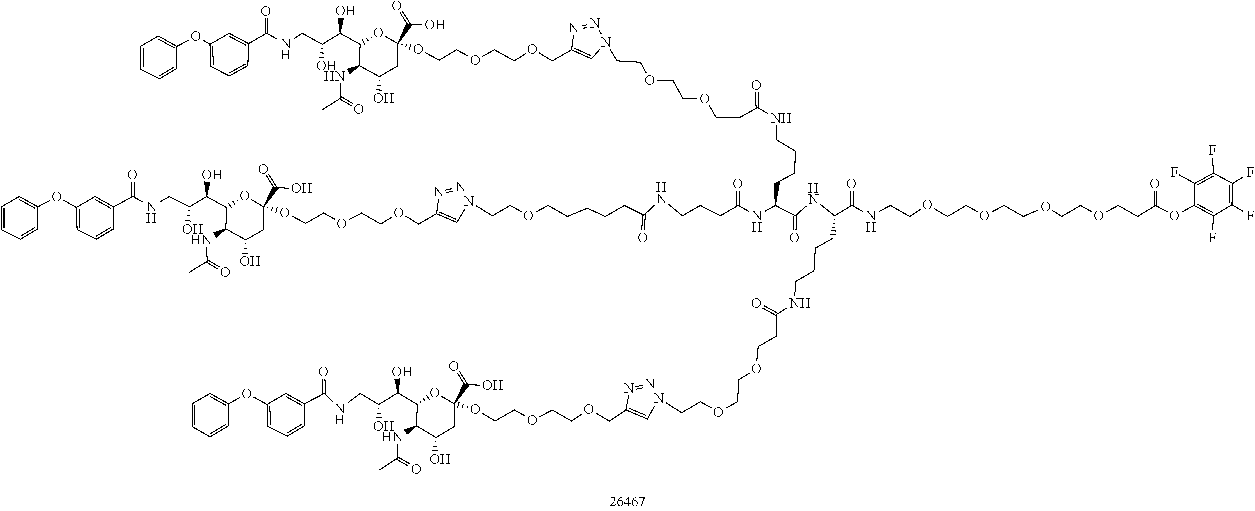

Synthesis of Cpd. No. 26467

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 23.7 mg, 0.0164 mmol) and (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 3.10 eq, 37.9 mg, 0.0508 mmol) in NMP (0.6 mL) in a 1 dram vial with a stirbar was added tetrakis(acetonitrile)copper(I) hexafluorophosphate (7.50 eq, 45.8 mg, 0.123 mmol). The resulting clear light yellow solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford Cpd. No. 26467 as a white solid. Yield: 36.8 mg, 70%; LCMS m/z 1610.6 [M/2+1]+; ¹H NMR (300 MHz, DMSO-d₆ with D₂O) δ 7.93 (s, 3H), 7.51 (d, J=7.5 Hz, 3H), 7.46-7.27 (m, 12H), 7.11 (t, J=8.0 Hz, 6H), 6.94 (d, J=7.9 Hz, 6H), 4.48-4.35 (m, 12H), 4.10-2.82 (m, 99H), 2.45-2.39 (m, 2H), 2.29-2.17 (m, 6H), 2.13-2.02 (m, 2H), 1.82 (s, 9H), 1.66-1.36 (m, 8H), 1.36-1.05 (m, 8H).

### (2RS,2′RS,4S R,4′SR,5RS,5′RS,6RS,6′RS)-2,2′-((((2R,2′R,3R,3′R,4S,4′S,5R,5′R,6R,6′R)—(((((SR)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(5-acetamido-6-((1RS,2RS)-1,2-dihydroxy-3-(3-phenoxybenzamido)propyl)-4-hydroxytetrahydro-2H-pyran-2-carboxylic acid) (Cpd. No. 26468)

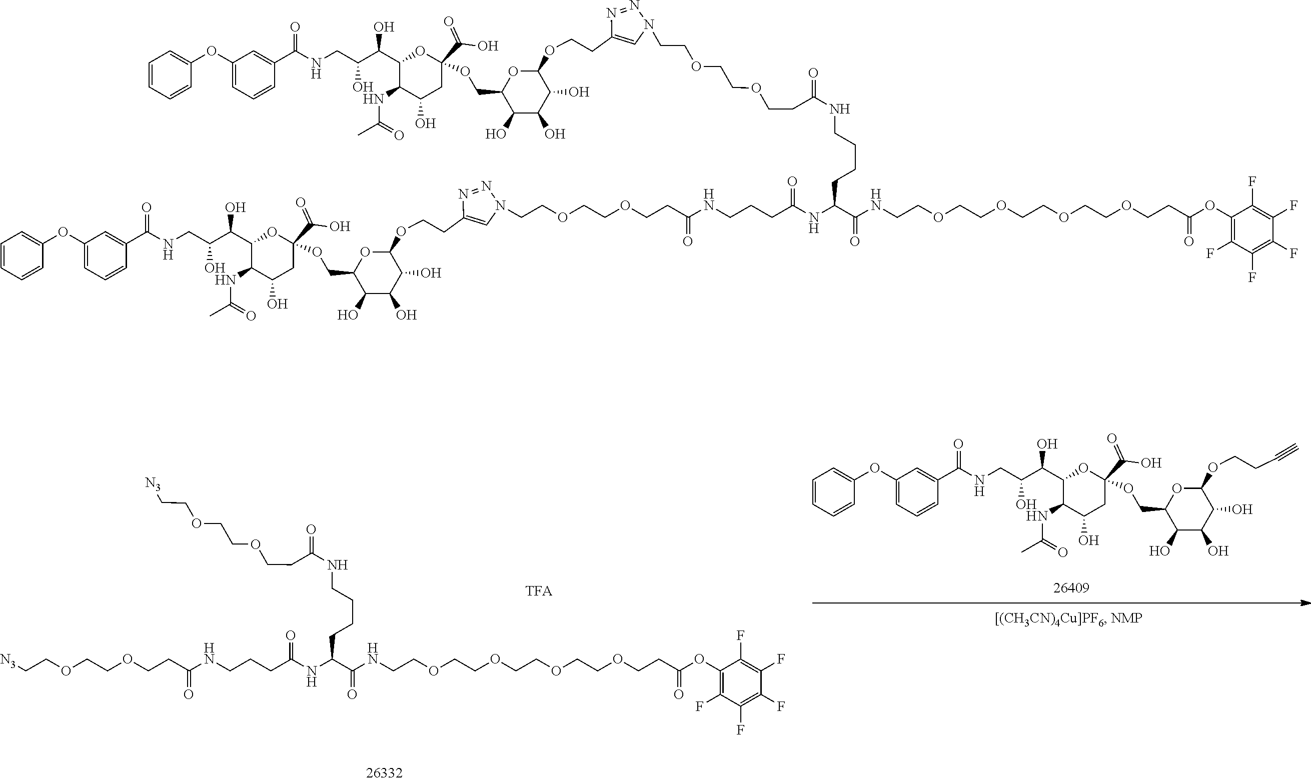

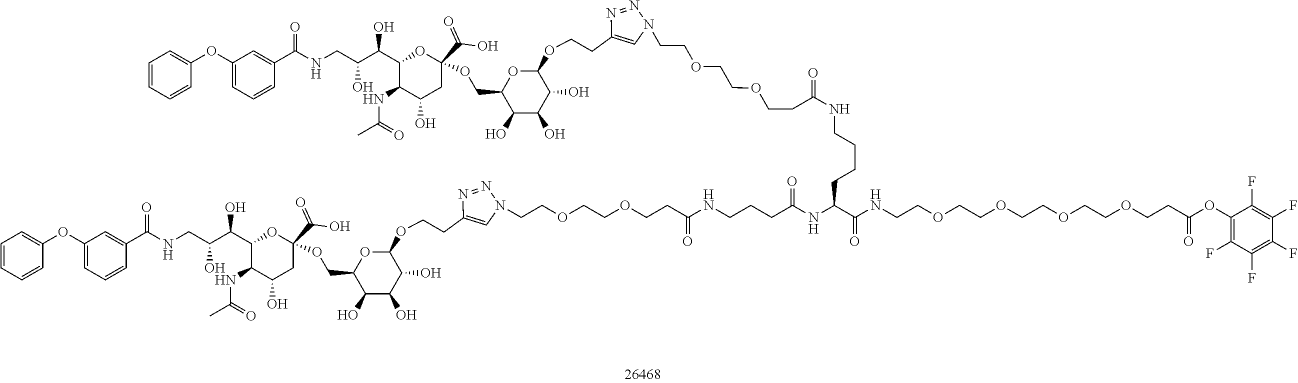 [SMILES: CC(=O)N[C@H]1[C@H]([C@H](O)[C@H](O)CNC(=O)c2cccc(Oc3ccccc3)c2)O[C@@](OC[C@H]2O[C@@H](OCCc3cn(CCOCCOCCC(=O)NCCCC[C@H](NC(=O)CCCNC(=O)CCOCCOCCn4cc(CCO[C@@H]5O[C@H](CO[C@]6(C(=O)O)C[C@H](O)[C@@H](NC(C)=O)[C@H]([C@H](O)[C@H](O)CNC(=O)c7cccc(Oc8ccccc8)c7)O6)[C@H](O)[C@H](O)[C@H]5O)nn4)C(=O)NCCOCCOCCOCCOCCC(=O)Oc4c(F)c(F)c(F)c(F)c4F)nn3)[C@H](O)[C@@H](O)[C@H]2O)(C(=O)O)C[C@@H]1O]

### Synthesis of (2RS,2′RS,4SR,4′SR,5RS,5′RS,6RS,6′RS)-2,2′-((((2R,2′R,3R,3′R,4S,4′S,5R,5′R,6R,6′R)—(((((SR)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(5-acetamido-6-((1RS,2 RS)-1,2-dihydroxy-3-(3-phenoxybenzamido)propyl)-4-hydroxytetrahydro-2H-pyran-2-carboxylic acid) (Cpd. No. 26468)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 18.4 mg, 0.0163 mmol) and (2RS,4SR,5RS,6RS)-5-acetamido-2-(((2R,3R,4S,5R,6R)-6-(but-3-yn-1-yloxy)-3,4,5-trihydroxytetrahydro-2H-pyran-2-yl)methoxy)-6-((1RS,2 RS)-1,2-dihydroxy-3-(3-phenoxybenzamido)propyl)-4-hydroxytetrahydro-2H-pyran-2-carboxylic acid (26409, 2.10 eq, 24.6 mg, 0.0342 mmol) in NMP (0.5 mL) in a 1 dram vial with a stirbar was added tetrakis(acetonitrile)copper(I) hexafluorophosphate (5.00 eq, 30.4 mg, 0.0815 mmol The resulting colourless solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford (2RS,2′RS,4SR,4′SR,5RS,5′RS,6RS,6′RS)-2,2′-((((2R,2′R,3R,3′R,4S,4′S,5R,5′R,6R,6′R)—(((((SR)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(5-acetamido-6-((1RS,2RS)-1,2-dihydroxy-3-(3-phenoxybenzamido)propyl)-4-hydroxytetrahydro-2H-pyran-2-carboxylic acid) (Cpd. No. 26468) as a white solid. Yield: 28.2 mg, 71%; LCMS m/z 1226.8 [M/2+1]+; ¹H NMR (300 MHz, DMSO-d₆ with D₂O) δ 7.79 (s, 2H), 7.51 (d, J=7.8 Hz, 2H), 7.45-7.30 (m, 8H), 7.16-7.05 (m, 4H), 6.94 (d, J=8.0 Hz, 4H), 4.42-4.33 (m, 4H), 4.15-2.69 (m, 77H), 2.44-2.39 (m, 2H), 2.30-2.18 (m, 4H), 2.14-2.02 (m, 2H), 1.82 (s, 6H), 1.64-1.38 (m, 6H), 1.38-1.07 (m, 4H).

### (2RS,2′RS,4RS,4′RS,5SR,5′SR,6SR,6′SR)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(5-acetamido-6-((1SR,2SR)-1,2-dihydroxy-3-(3-phenoxybenzamido)propyl)-4-hydroxytetrahydro-2H-pyran-2-carboxylic acid) (Cpd. No. 26469)

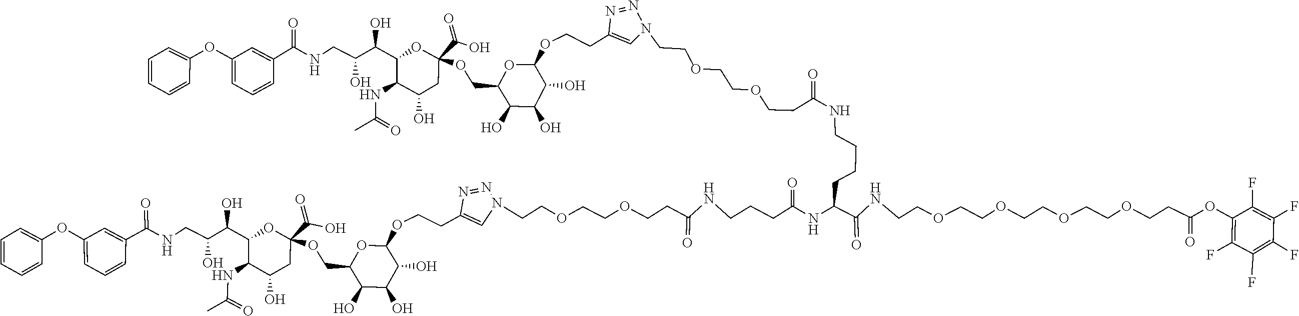

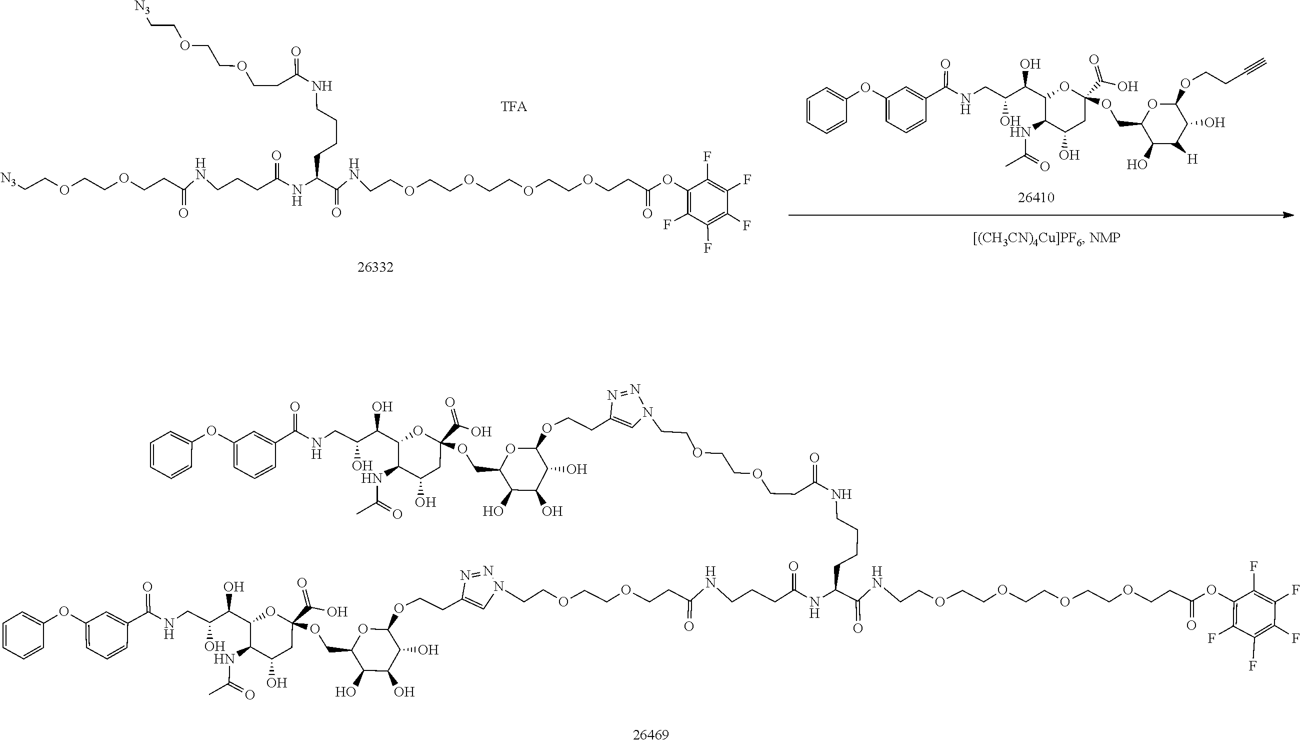

### Synthesis of (2RS,2′RS,4RS,4′RS,5SR,5′SR,6SR,6′SR)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(5-acetamido-6-((1SR,2SR)-1,2-dihydroxy-3-(3-phenoxybenzamido)propyl)-4-hydroxytetrahydro-2H-pyran-2-carboxylic acid) (Cpd. No. 26469)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 19.3 mg, 0.0171 mmol) and (2RS,4RS,5SR,6SR)-5-acetamido-2-(((2R,3R,4S,5R,6R)-6-(but-3-yn-1-yloxy)-3,4,5-trihydroxytetrahydro-2H-pyran-2-yl)methoxy)-6-((1SR,2SR)-1,2-dihydroxy-3-(3-phenoxybenzamido)propyl)-4-hydroxytetrahydro-2H-pyran-2-carboxylic acid (26410, 2.10 eq, 25.8 mg, 0.0359 mmol) in NMP (0.5 mL) in a 1 dram vial with a stirbar was added tetrakis(acetonitrile)copper(I) hexafluorophosphate (5.00 eq, 31.8 mg, 0.0854 mmol). The resulting colourless solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford (2RS,2′RS,4RS,4′RS,5SR,5′SR,6SR,6′SR)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(5-acetamido-6-((1SR,2SR)-1,2-dihydroxy-3-(3-phenoxybenzamido)propyl)-4-hydroxytetrahydro-2H-pyran-2-carboxylic acid) (Cpd. No. 26469) as a white solid. Yield: 26.0 mg, 62%; LCMS m/z 1226.8 [M/2+1]+; ¹H NMR (300 MHz, DMSO-d₆ with D₂O) δ 7.92 (bs, 2H), 7.83 (s, 2H), 7.54 (d, J=7.6 Hz, 2H), 7.44 (t, J=7.8 Hz, 2H), 7.41-7.31 (m, 6H), 7.13 (t, J=7.2 Hz, 4H), 6.96 (d, J=8.0 Hz, 4H), 4.45-4.32 (m, 4H), 3.95-2.70 (m, 77H), 2.31-2.02 (m, 8H), 1.82 (s, 6H), 1.63-1.37 (m, 6H), 1.37-1.07 (m, 4H).

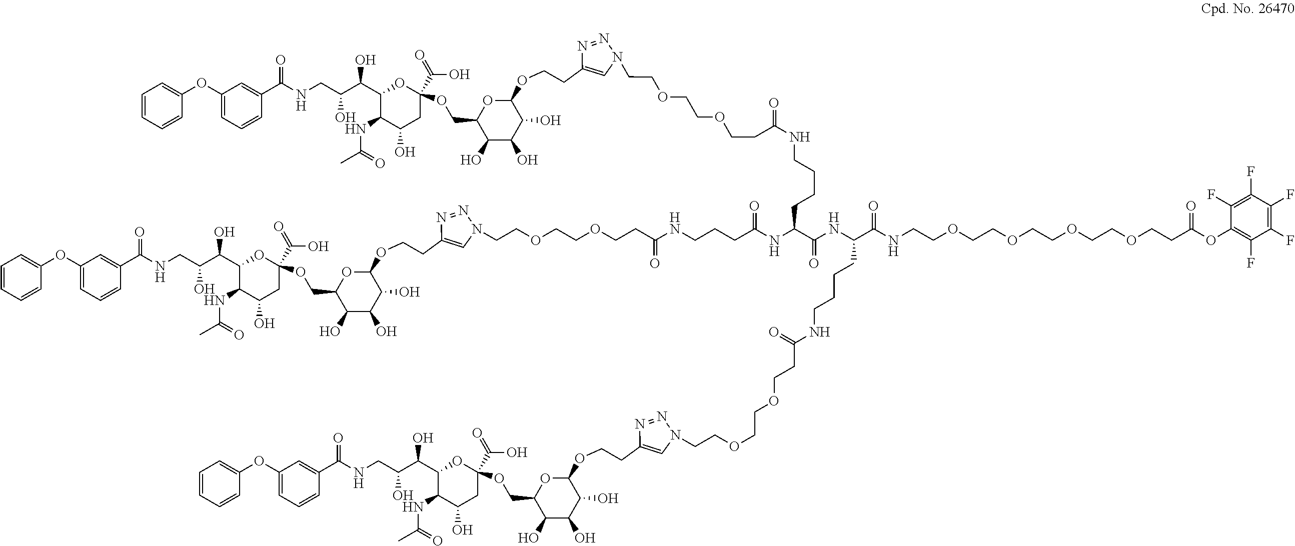

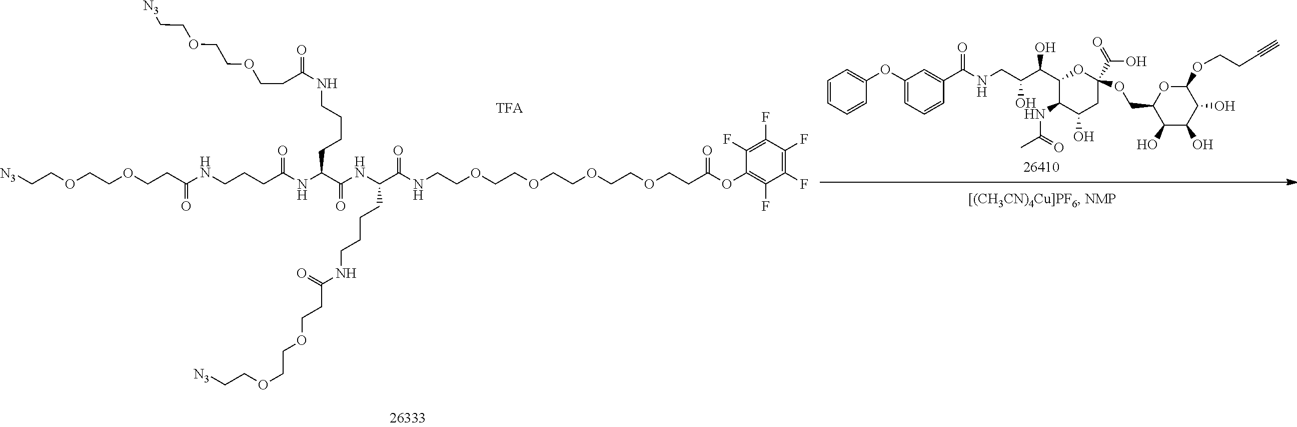 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F ; C#CCCO[C@@H]1O[C@H](CO[C@@]2(C(=O)O)C[C@H](O)[C@@H](NC(C)=O)[C@H]([C@H](O)[C@H](O)CNC(=O)c3cccc(Oc4ccccc4)c3)O2)[C@H](O)[C@H](O)[C@H]1O]

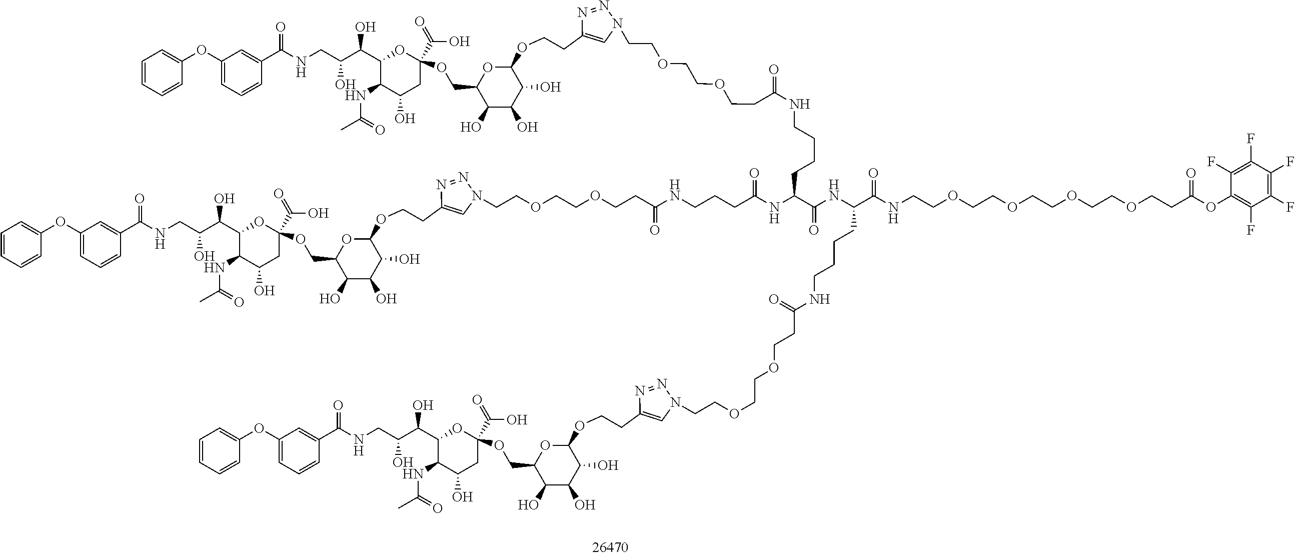

Synthesis of Cpd. No. 26470

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 21.6 mg, 0.0149 mmol) and (2RS,4RS,5SR,6SR)-5-acetamido-2-(((2R,3R,4S,5R,6R)-6-(but-3-yn-1-yloxy)-3,4,5-trihydroxytetrahydro-2H-pyran-2-yl)methoxy)-6-((1SR,2SR)-1,2-dihydroxy-3-(3-phenoxybenzamido)propyl)-4-hydroxytetrahydro-2H-pyran-2-carboxylic acid (26410, 3.10 eq, 33.3 mg, 0.0463 mmol) in NMP (0.6 mL) in a 1 dram vial with a stirbar was added tetrakis(acetonitrile)copper(I) hexafluorophosphate (7.50 eq, 41.8 mg, 0.112 mmol). The resulting clear yellow solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford Cpd. No. 26470 as a white solid. Yield: 27.3 mg, 52%; LCMS m/z 1742.4 [M/2+1]+; ¹H NMR (300 MHz, DMSO-d₆ with D₂O) δ 7.92 (bs, 3H), 7.82 (s, 3H), 7.52 (d, J=7.7 Hz, 3H), 7.43 (t, J=7.9 Hz, 3H), 7.39-7.30 (m, 9H), 7.12 (t, J=7.2 Hz, 6H), 6.95 (d, J=7.9 Hz, 6H), 4.42-4.32 (m, 6H), 4.17-2.70 (m, 108H), 2.31-2.02 (m, 10H), 1.82 (s, 9H), 1.66-1.38 (m, 8H), 1.38-1.05 (m, 8H). Yield: 12.7 mg, 24%; ¹H NMR (300 MHz, DMSO-d₆ with D₂O) δ 7.92 (bs, 3H), 7.83 (s, 3H), 7.54 (d, J=7.7 Hz, 3H), 7.45 (t, J=7.9 Hz, 3H), 7.40-7.31 (m, 9H), 7.13 (t, J=7.0 Hz, 6H), 6.96 (d, J=8.0 Hz, 6H), 4.42-4.33 (m, 6H), 3.96-2.70 (m, 108H), 2.32-2.03 (m, 10H), 1.82 (s, 9H), 1.66-1.39 (m, 8H), 1.39-1.07 (m, 8H).

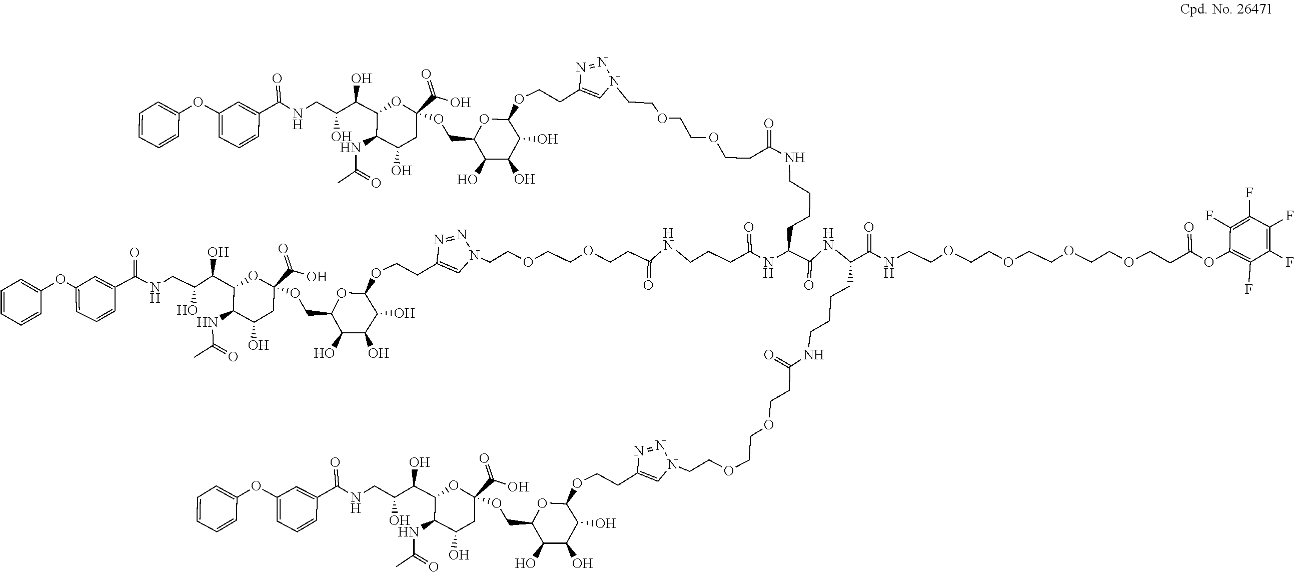

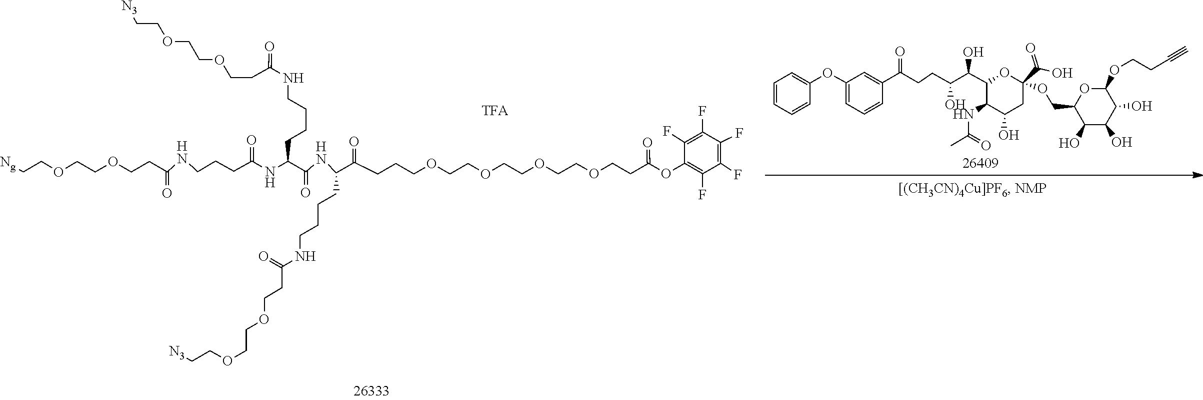

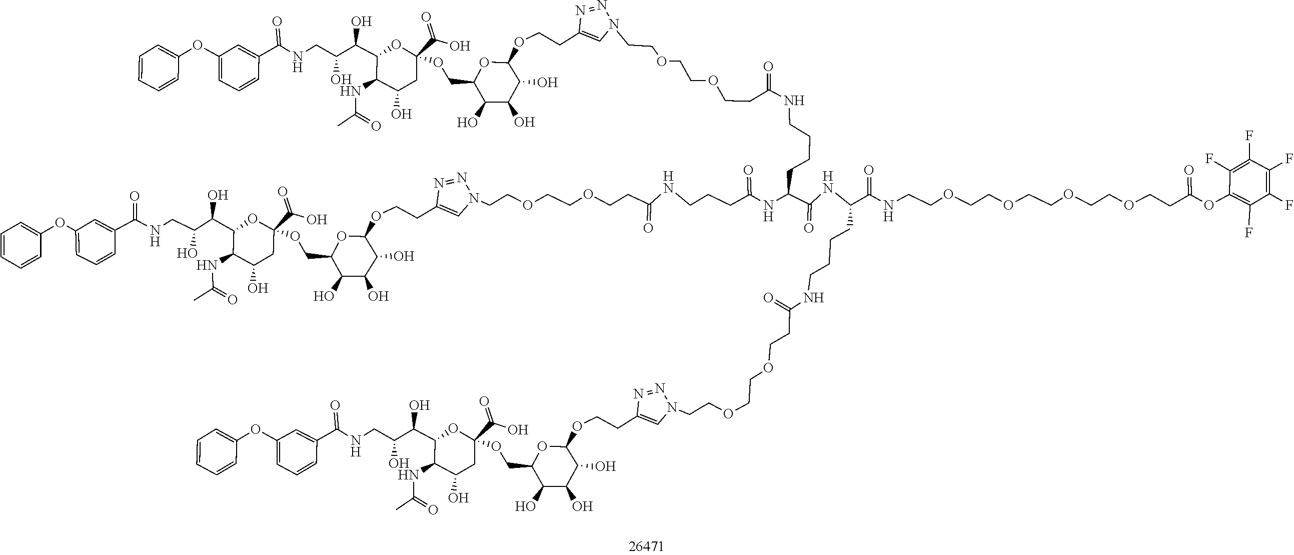

Synthesis of Cpd. No. 26471

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 17.1 mg, 0.0119 mmol) and (2RS,4SR,5RS,6RS)-5-acetamido-2-(((2R,3R,4S,5R,6R)-6-(but-3-yn-1-yloxy)-3,4,5-trihydroxytetrahydro-2H-pyran-2-yl)methoxy)-6-((1RS,2 RS)-1,2-dihydroxy-3-(3-phenoxybenzamido)propyl)-4-hydroxytetrahydro-2H-pyran-2-carboxylic acid (26409, 3.10 eq, 26.4 mg, 0.0368 mmol) in NMP (0.6 mL) in a 1 dram vial with a stirbar was added tetrakis(acetonitrile)copper(I) hexafluorophosphate (7.50 eq, 33.1 mg, 0.0889 mmol). The resulting clear yellow solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford Cpd. No. 26471 as a white solid. Yield: 19.7 mg, 48%; LCMS m/z 1742.5 [M/2+1]+; ¹H NMR (300 MHz, DMSO-d₆ with D₂O) δ 7.94 (bs, 3H), 7.81 (s, 3H), 7.53 (d, J=7.7 Hz, 3H), 7.47-7.31 (m, 12H), 7.17-7.05 (m, 6H), 6.95 (d, J=8.0 Hz, 6H), 4.44-4.30 (m, 6H), 4.16-4.09 (m, 2H), 3.90-2.69 (m, 106H), 2.45-2.37 (m, 2H), 2.31-2.16 (m, 6H), 2.14-2.01 (m, 2H), 1.82 (s, 9H), 1.65-1.38 (m, 8H), 1.38-1.04 (m, 8H). Yield: 8.1 mg, 20%; ¹H NMR (300 MHz, DMSO-d₆ with D₂O) δ 7.93 (bs, 3H), 7.78 (s, 3H), 7.50 (d, J=7.6 Hz, 3H), 7.45-7.29 (m, 12H), 7.16-7.03 (m, 6H), 6.94 (d, J=8.0 Hz, 6H), 4.41-4.32 (m, 6H), 4.15-2.70 (m, 108H), 2.32-2.18 (m, 8H), 2.14-2.04 (m, 2H), 1.82 (s, 9H), 1.66-1.38 (m, 8H), 1.38-1.05 (m, 8H).

### (2S,4S,5R,6R)-5-acetamido-6-((1R,2R)-1,2-dihydroxy-3-(3-phenoxybenzamido)propyl)-2,4-dihydroxytetrahydro-2H-pyran-2-carboxylic acid (Cpd. No. 26473)

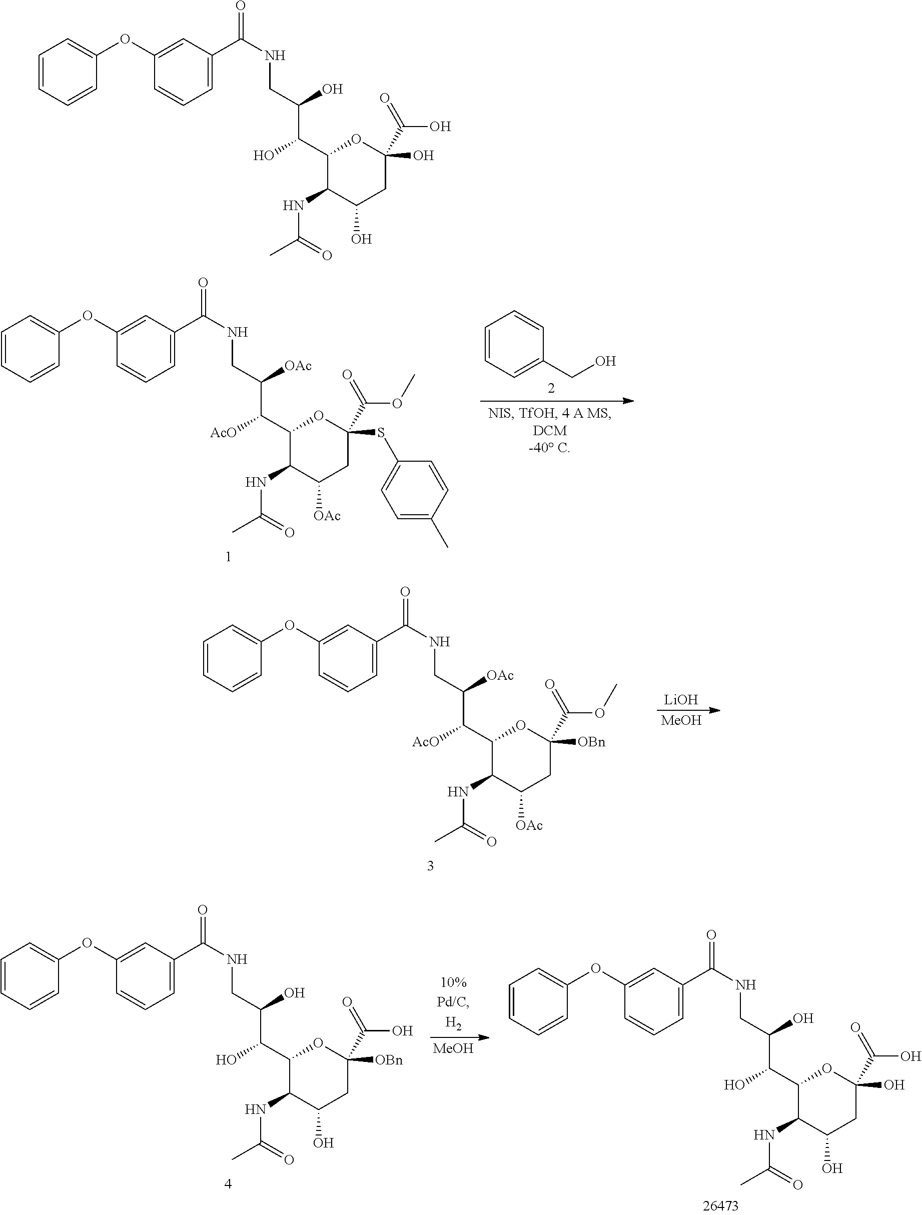

### Synthesis of (1R,2R)-1-((2R,3R,4S,6S)-3-acetamido-4-acetoxy-6-(benzyloxy)-6-(methoxycarbonyl)tetrahydro-2H-pyran-2-yl)-3-(3-phenoxybenzamido)propane-1,2-diyl diacetate (3)

A suspension of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-phenoxybenzamido)propane-1,2-diyl diacetate (1, 0.50 g, 0.66 mmol), phenylmethanol (2, 0.203 mL, 2.0 mmol), and activated 4 Å powdered molecular sieves (0.50 g, 100% w/w) in anhydrous dichloromethane (10.0 mL) was stirred at room temperature for 15 h under nitrogen atmosphere. The reaction mixture was cooled to −40° C. followed by the addition of 1-iodopyrrolidine-2,5-dione (0.374 g, 1.66 mmol) and trifluoromethanesulfonic acid (0.058 mL, 0.66 mmol). The reaction was stirred at −40° C. for 1 h and progress was monitored by TLC and LCMS. After completion, the reaction mixture was quenched with triethyl amine (0.1 mL, neutral pH) and warmed to room temperature. The reaction mixture was filtered through a sintered funnel and washed with dichloromethane. The filtrate was washed with a saturated solution of sodium bicarbonate, dried over sodium sulfate, filtered, and concentrated under reduced pressure to obtain a crude residue. The residue was purified via column chromatography (45-60% ethyl acetate in hexanes) to afford (1R,2R)-1-((2R,3R,4S,6S)-3-acetamido-4-acetoxy-6-(benzyloxy)-6-(methoxycarbonyl)tetrahydro-2H-pyran-2-yl)-3-(3-phenoxybenzamido)propane-1,2-diyl diacetate (3) as an off white solid. Yield: 0.30 g, 61.31%; LCMS (ESI) m/z 7354.22 [M+1]⁺.

### Synthesis of (2R,4S,5R,6R)-5-acetamido-2-(benzyloxy)-6-((1R,2R)-1,2-dihydroxy-3-(3-phenoxybenzamido)propyl)-4-hydroxytetrahydro-2H-pyran-2-carboxylic acid (4)

To a stirred solution of (1R,2R)-1-((2R,3R,4S,6S)-3-acetamido-4-acetoxy-6-(benzyloxy)-6-(methoxycarbonyl)tetrahydro-2H-pyran-2-yl)-3-(3-phenoxybenzamido)propane-1,2-diyl diacetate (3, 0.30 g, 0.408 mmol) in methanol (10.0 mL) at 0° C. was added lithium hydroxide (0.048 g, 2.04 mmol). The mixture was stirred and allowed to warm to room temperature. After completion, Dowex-Hydrogen form was added to the reaction mass up to pH 6. The reaction mixture was filtered through a sintered funnel and the filtrate was concentrated on a rotary evaporator to afford crude (2S,4S,5R,6R)-5-acetamido-2-(benzyloxy)-6-((1R,2R)-1,2-dihydroxy-3-(3-phenoxybenzamido)propyl)-4-hydroxytetrahydro-2H-pyran-2-carboxylic acid (4) as a white solid. Yield: 0.24 g, crude; LCMS (ESI) m/z 595.22 [M+1]⁺.

### Synthesis of (2S,4S,5R,6R)-5-acetamido-6-((1R,2R)-1,2-dihydroxy-3-(3-phenoxybenzamido)propyl)-2,4-dihydroxytetrahydro-2H-pyran-2-carboxylic acid (Cpd. No. 26473)

To a stirred solution of (2S,4S,5R,6R)-5-acetamido-2-(benzyloxy)-6-((1R,2R)-1,2-dihydroxy-3-(3-phenoxybenzamido)propyl)-4-hydroxytetrahydro-2H-pyran-2-carboxylic acid (4, 0.24 g, 0.403 mmol) in methanol (3.0 mL) was added 10% Pd/C (0.12 g, 50% w/w) at room temperature. The reaction was hydrogenated using balloon pressure of H₂ gas for 12 h. The reaction was monitored by LC-MS and TLC and after completion, the reaction was filtered through celite and the filtrate was concentrated. The residue was purified via preparatory HPLC (17-35% acetonitrile in water with 0.1% TFA). Fractions containing the desired product were combined and lyophilized to dryness to afford (2S,4S,5R,6R)-5-acetamido-6-((1R,2R)-1,2-dihydroxy-3-(3-phenoxybenzamido)propyl)-2,4-dihydroxytetrahydro-2H-pyran-2-carboxylic acid (Cpd. No. 26473) as the TFA salt as a white solid. Yield: 0.078 g, 38.31%; LCMS (ESI) m/z 505.35 [M+1]⁺; ¹H NMR (400 MHz, Methanol-d₄) δ 8.42-8.40 (m, 1H), 8.09 (d, J=8.4 Hz, 1H), 7.56 (d, J=7.6 Hz, 1H), 7.46-7.42 (m, 2H), 7.39-7.35 (m, 2H), 7.16-7.12 (m, 2H), 7.02-7.00 (m, 2H), 4.06-4.00 (m, 2H), 3.88-3.75 (m, 3H), 3.51-3.42 (m, 2H), 2.21 (dd, J=12.4 & 4.8 Hz, 1H), 1.97 (s, 3H), 1.83 (t, J=12.4 Hz, 1H).

### (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 26474)

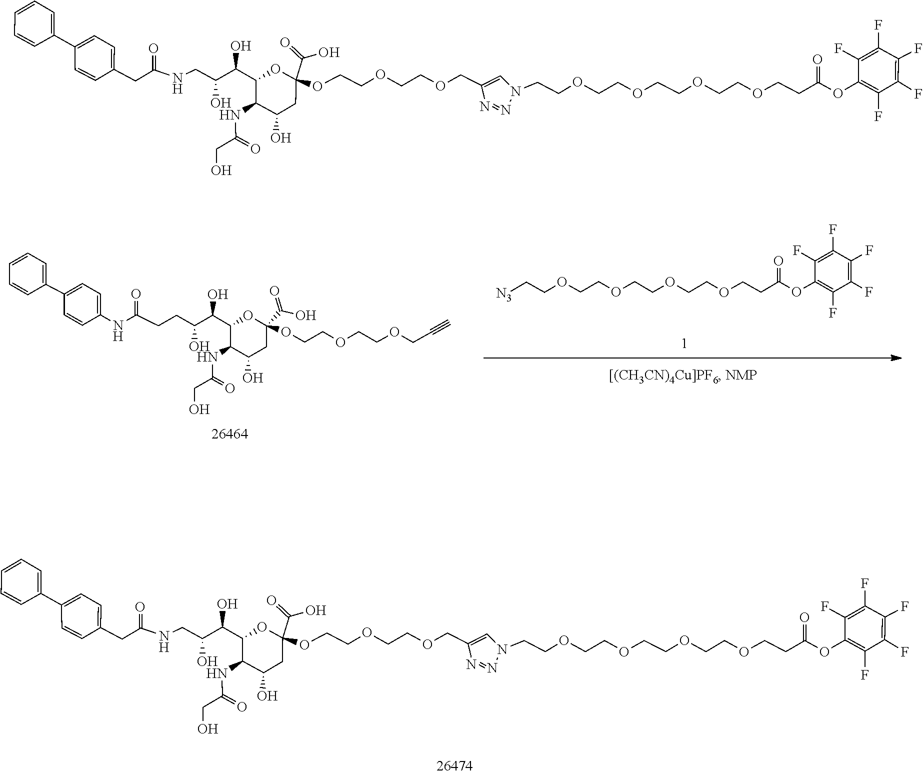 [SMILES: O=C(Cc1ccc(-c2ccccc2)cc1)NC[C@@H](O)[C@@H](O)[C@@H]1O[C@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO ; C#CCOCCOCCO[C@@]1(C(=O)O)C[C@H](O)[C@@H](NC(=O)CO)[C@H]([C@H](O)[C@H](O)CCC(=O)Nc2ccc(-c3ccccc3)cc2)O1 ; [N-]=[N+]=NCCOCCOCCOCCOCCC(=O)Oc1c(F)c(F)c(F)c(F)c1F ; O=C(Cc1ccc(-c2ccccc2)cc1)NC[C@@H](O)[C@@H](O)[C@@H]1O[C@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO]

### Synthesis of (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 26474)

To a solution of (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26464, 1.00 eq, 28.0 mg, 0.0434 mmol) in NMP (0.3 mL) in a 1 dram vial with a stirbar was added a solution of perfluorophenyl 1-azido-3,6,9,12-tetraoxapentadecan-15-oate (1, 1.10 eq, 21.9 mg, 0.0478 mmol) in NMP (0.3 mL) followed by tetrakis(acetonitrile)copper(I) hexafluorophosphate (2.50 eq, 40.5 mg, 0.109 mmol). The resulting clear green solution was capped and stirred at room temperature for 10 min. The reaction was diluted with acetic acid, filtered, and purified via preparatory HPLC (15-65% acetonitrile in water with 0.1% TFA). Fractions containing the desired product were combined and lyophilized to dryness to afford (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. No. 26474) as a white solid. Yield: 10.7 mg, 22%; LCMS m/z 1102.7 [M+1]+; ¹H NMR (300 MHz, DMSO-d₆ with D₂O) δ 7.96 (s, 1H), 7.69 (d, J=8.8 Hz, 1H), 7.61-7.48 (m, 4H), 7.41 (t, J=7.5 Hz, 2H), 7.34-7.24 (m, 3H), 4.52-4.38 (m, 3H), 4.02-2.69 (m, 38H), 2.31-2.07 (m, 1H), 1.54-1.38 (in, 1H).

### (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-dihydroxypropyl)-4-hydroxy-5-(2-hydroxyacetamido)tetrahydro-2H-pyran-2-carboxylic acid) (Cpd. No. 26475)

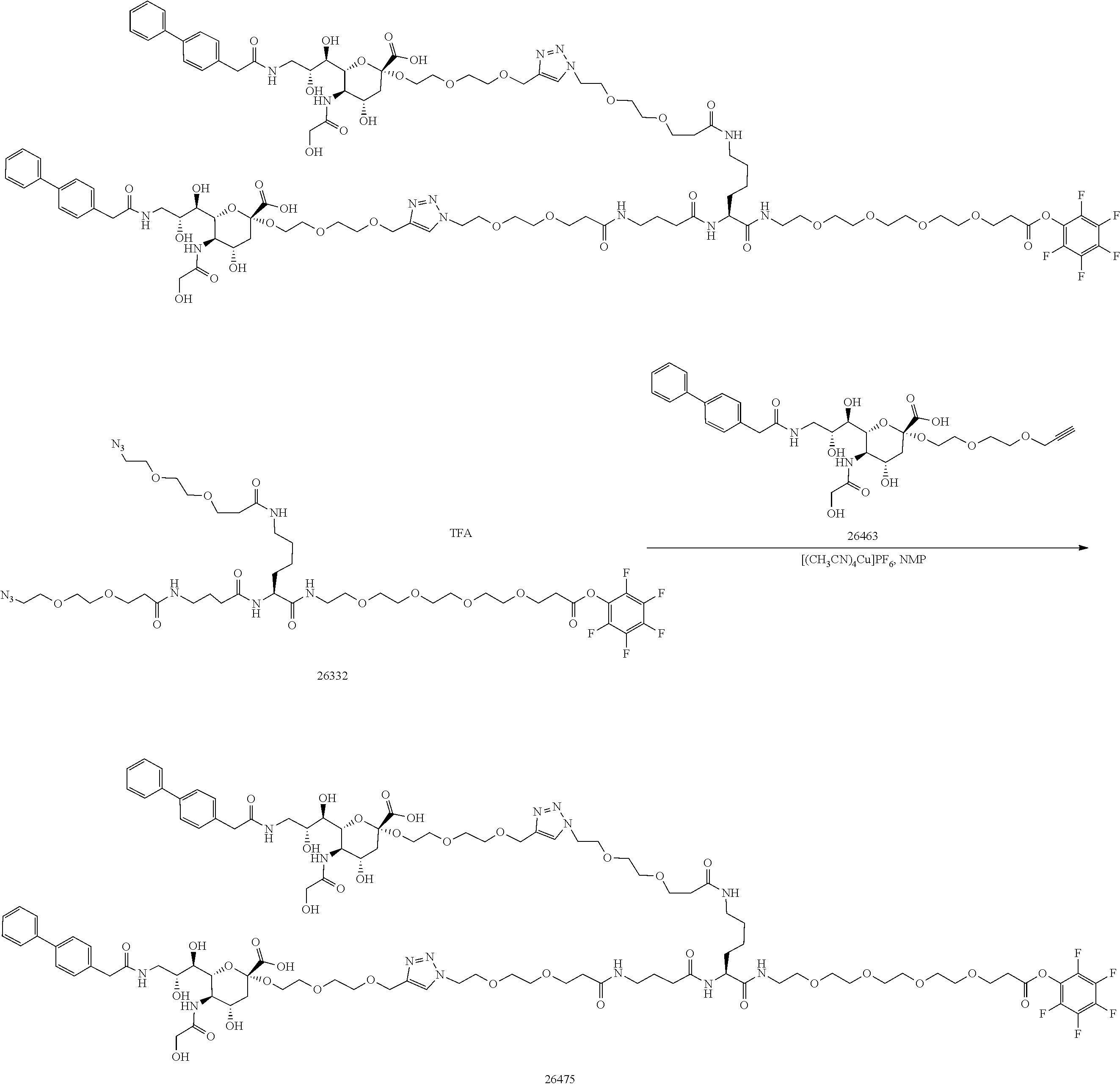

### Synthesis of (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26475)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 21.2 mg, 0.0188 mmol) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 2.10 eq, 25.4 mg, 0.0394 mmol)) in NMP (0.6 mL) in a 1 dram vial with a stirbar was added tetrakis(acetonitrile)copper(I) hexafluorophosphate (5.00 eq, 35.0 mg, 0.0939 mmol). The resulting clear yellow solution was capped and stirred at room temperature for 10 min (slowly turned green). The reaction was diluted with a mixture of 70% acetic acid in NMP, filtered, and purified via preparatory HPLC (15-60% acetonitrile in water with 0.1% TFA). Fractions containing the desired product were combined and lyophilized to dryness to afford (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26475) as a white solid. Yield: 30.2 mg, 70%; ¹H NMR (300 MHz, DMSO-d₆ with D₂O) δ 7.96 (s, 2H), 7.80 (d, J=7.1 Hz, 2H), 7.62-7.50 (m, 8H), 7.41 (t, J=7.5 Hz, 4H), 7.35-7.26 (m, 6H), 4.50-4.38 (m, 8H), 4.13-2.87 (m, 79H), 2.47-2.40 (m, 2H), 2.30-2.19 (m, 4H), 2.08 (t, J=7.6 Hz, 2H), 1.62-1.08 (m, 10H).

### (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(6-((1S,2S)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26476)

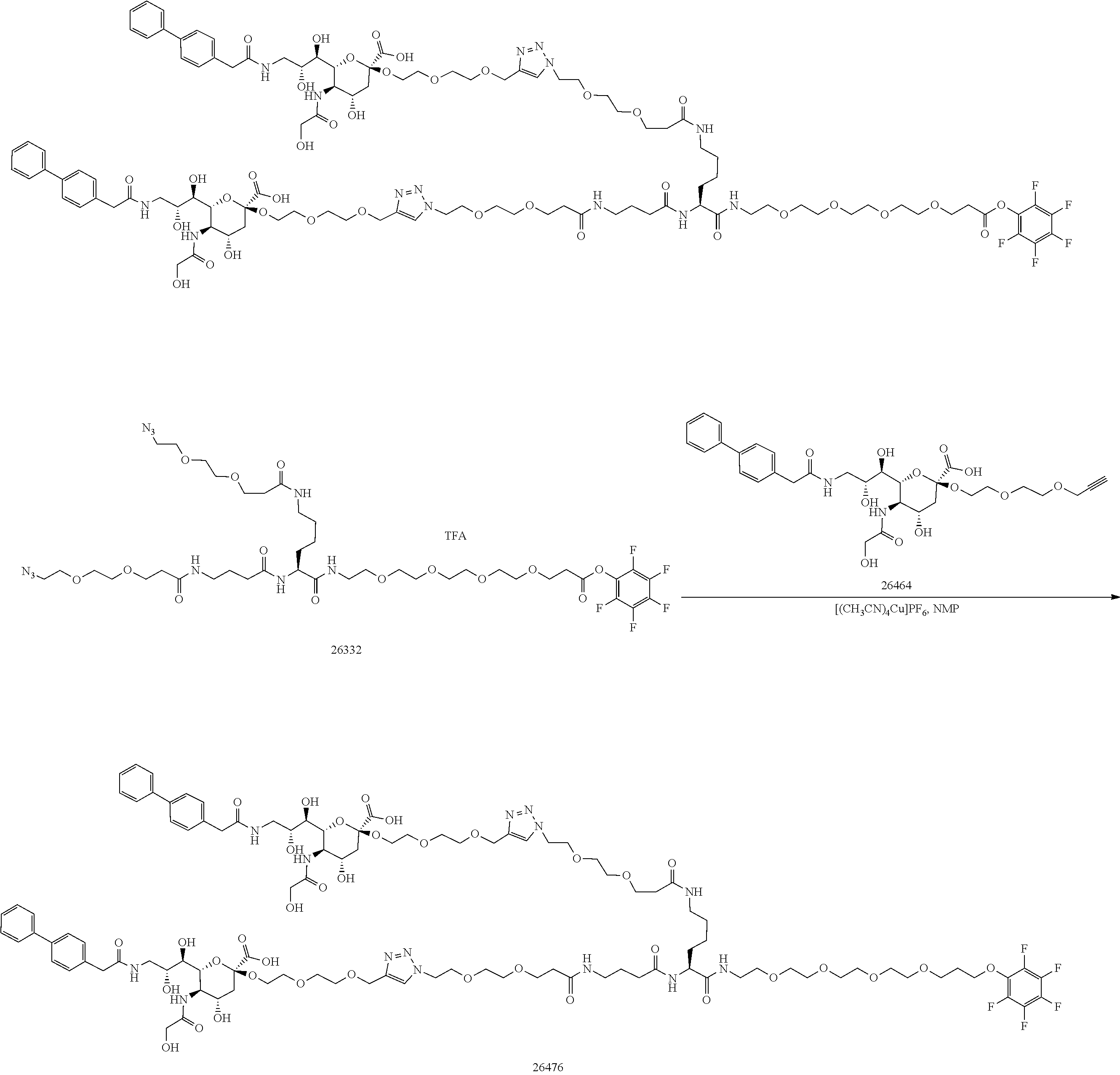

### Synthesis of (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(6-((1S,2S)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26476)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 21.9 mg, 0.0194 mmol) and (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26464, 2.10 eq, 26.3 mg, 0.0407 mmol) in NMP (0.6 mL) in a 1 dram vial with a stirbar was added tetrakis(acetonitrile)copper(I) hexafluorophosphate (5.00 eq, 36.1 mg, 0.0970 mmol). The resulting clear yellow solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(6-((1S,2S)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Cpd. No. 26476) as a white solid. Yield: 14.6 mg, 33%; LCMS m/z 1152.7 [M/2+1]+; ¹H NMR (300 MHz, DMSO-d₆ with D₂O) δ 7.95 (s, 2H), 7.74 (d, J=8.9 Hz, 2H), 7.62-7.48 (m, 8H), 7.41 (t, J=7.6 Hz, 4H), 7.35-7.24 (m, 6H), 4.51-4.39 (m, 7H), 4.02-2.70 (m, 80H), 2.30-2.03 (m, 8H), 1.62-1.37 (m, 6H), 1.37-1.08 (m, 4H).

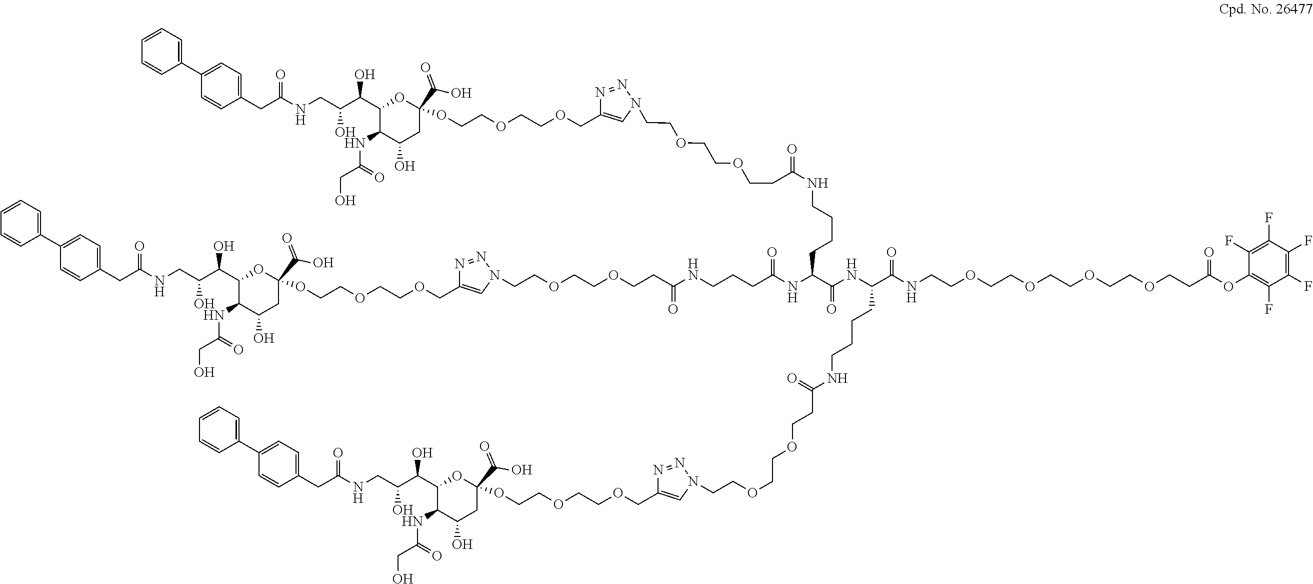 [SMILES: O=C(CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCCC[C@H](NC(=O)CCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)N[C@@H](CCCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)NCCOCCOCCOCCOCCC(=O)Oc1c(F)c(F)c(F)c(F)c1F]

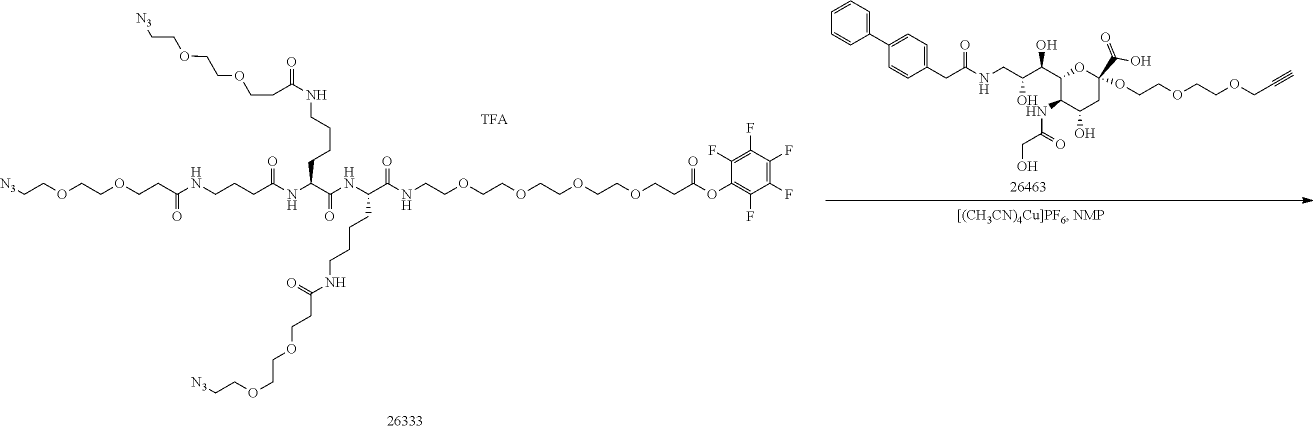 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F ; C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1]

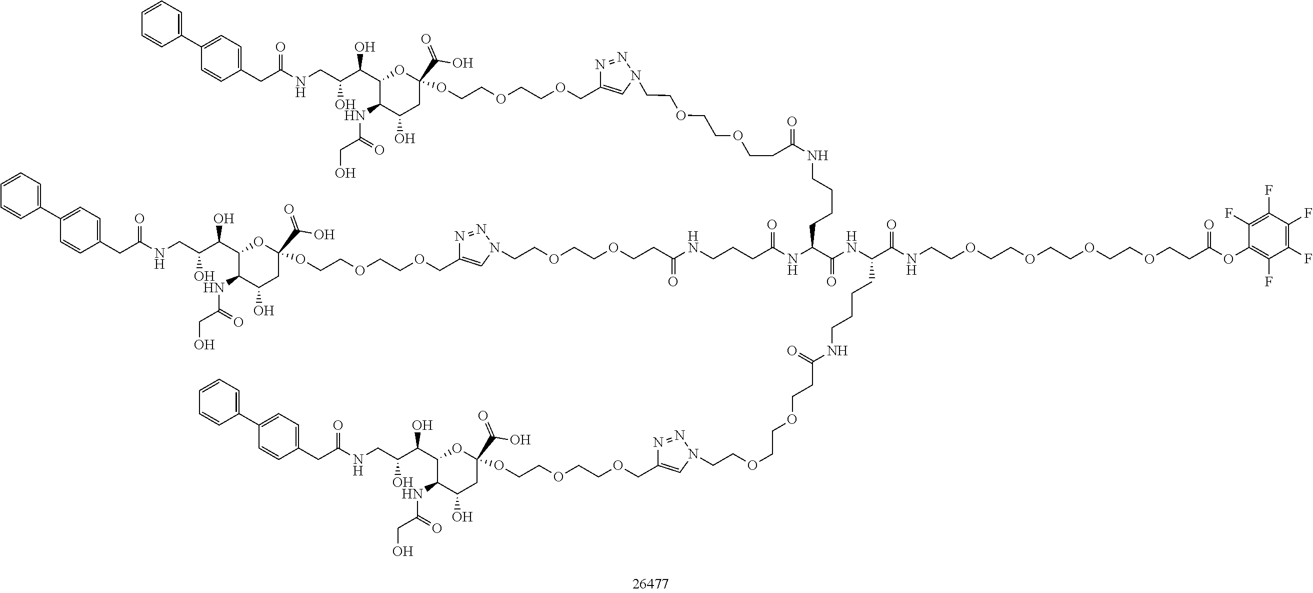 [SMILES: O=C(CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCCC[C@H](NC(=O)CCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)N[C@@H](CCCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)NCCOCCOCCOCCOCCC(=O)Oc1c(F)c(F)c(F)c(F)c1F]

Synthesis of Cpd. No. 26477

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 22.5 mg, 0.0156 mmol) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 3.10 eq, 31.2 mg, 0.0484 mmol) in NMP (0.6 mL) in a 1 dram vial with a stirbar was added tetrakis(acetonitrile)copper(I) hexafluorophosphate (7.50 eq, 43.6 mg, 0.117 mmol). The resulting clear yellow solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford Cpd. No. 26477 as a white solid. Yield: 23.4 mg, 46%; ¹H NMR (300 MHz, DMSO-d₆ with D₂O) δ 7.94 (s, 3H), 7.77 (bs, 3H), 7.60-7.46 (m, 12H), 7.40 (t, J=7.6 Hz, 6H), 7.34-7.24 (m, 9H), 4.49-4.36 (m, 11H), 3.90-2.84 (m, 114H), 2.31-2.03 (m, 8H), 1.65-1.37 (m, 8H), 1.37-1.06 (m, 8H).

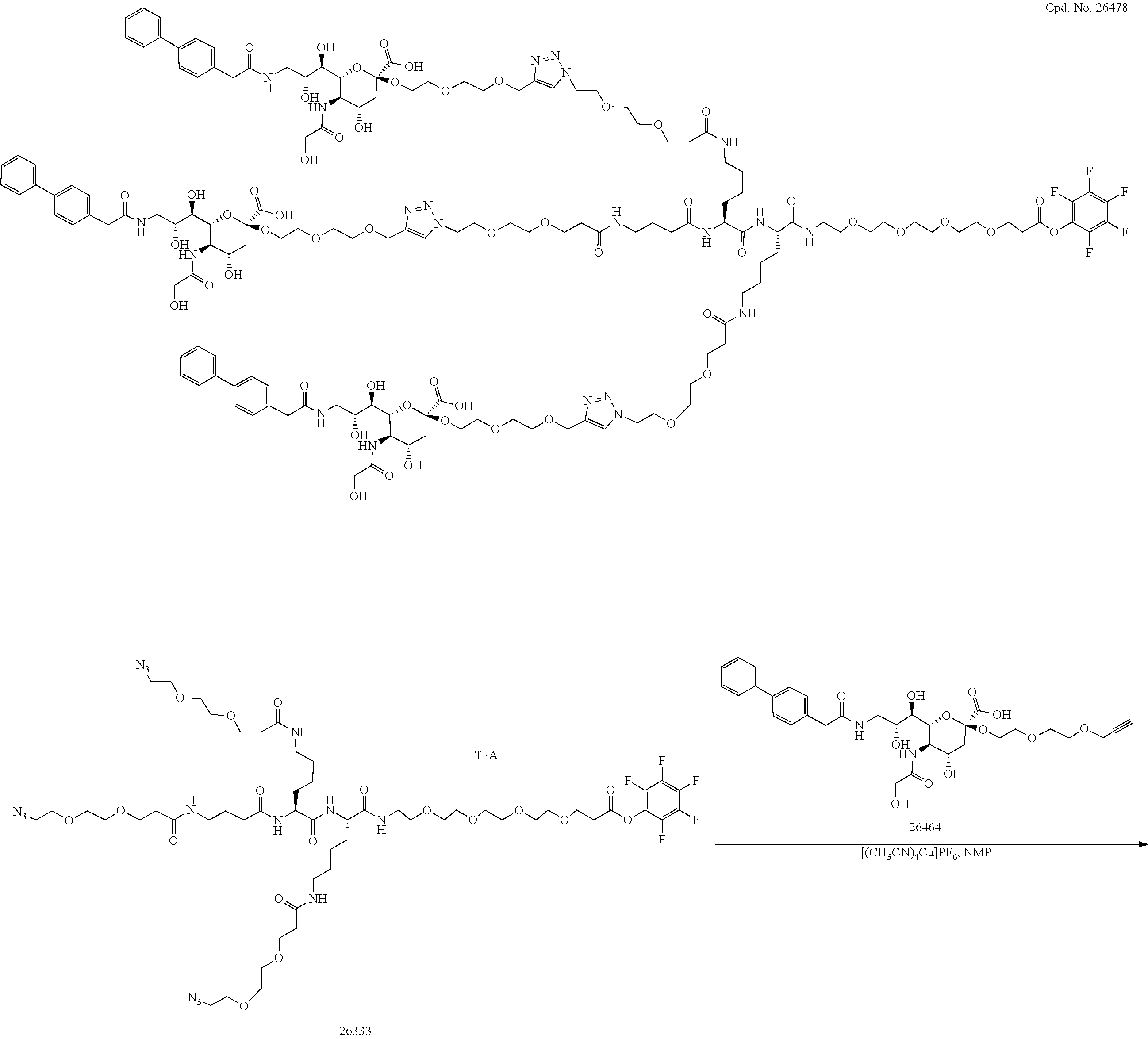

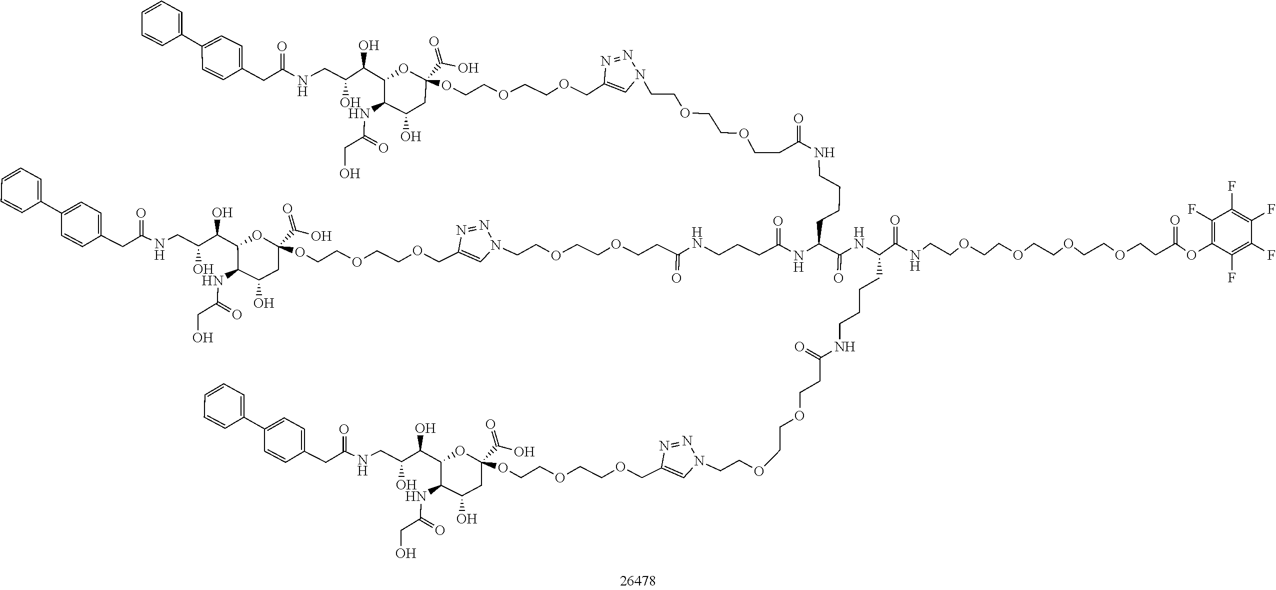 [SMILES: O=C(CCOCCOCCn1cc(COCCOCCO[C@@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCCC[C@H](NC(=O)CCCNC(=O)CCOCCOCCn1cc(COCCOCCO[C@@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)N[C@@H](CCCCNC(=O)CCOCCOCCn1cc(COCCOCCO[C@@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)NCCOCCOCCOCCOCCC(=O)Oc1c(F)c(F)c(F)c(F)c1F]

Synthesis of Cpd. No. 26478

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 24.3 mg, 0.0168 mmol) and (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26464, 3.10 eq, 33.7 mg, 0.0522 mmol) in NMP (0.6 mL) in a 1 dram vial with a stirbar was added tetrakis(acetonitrile)copper(I) hexafluorophosphate (7.50 eq, 47.1 mg, 0.126 mmol). The resulting clear yellow solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford Cpd. No. 26478 as a white solid. Yield: 23.4 mg, 43%; LCMS m/z 1631.8 [M/2+1]+; ¹H NMR (300 MHz, DMSO-d₆ with D₂O) δ 7.96 (s, 3H), 7.74 (d, J=8.9 Hz, 3H), 7.62-7.48 (m, 12H), 7.41 (t, J=7.6 Hz, 6H), 7.35-7.25 (m, 9H), 4.51-4.38 (m, 11H), 3.90-2.70 (m, 110H), 2.32-2.03 (m, 12H), 1.65-1.37 (m, 8H), 1.37-1.06 (m, 8H).

### perfluorophenyl 1-(4-(13-hydroxy-2,5,8,11-tetraoxatridecyl)-1H-1,2,3-triazol-1-yl)-3,6,9,12-tetraoxapentadecan-15-oate (Cpd. No. 26530)

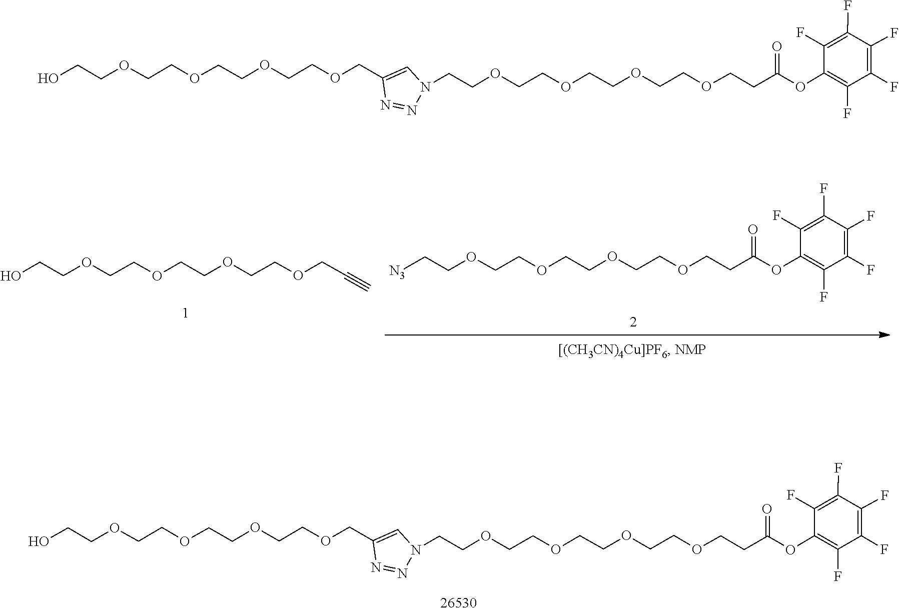

### Synthesis of perfluorophenyl 1-(4-(13-hydroxy-2,5,8,11-tetraoxatridecyl)-1H-1,2,3-triazol-1-yl)-3,6,9,12-tetraoxapentadecan-15-oate (Cpd. No. 26530)

To 3,6,9,12-tetraoxapentadec-14-yn-1-ol (1, 1.00 eq, 14.4 mg, 0.0620 mmol) in a 1 dram vial with a stirbar was added a solution of perfluorophenyl 1-azido-3,6,9,12-tetraoxapentadecan-15-oate (2, 1.10 eq, 31.2 mg, 0.0682 mmol) in NMP (0.5 mL) followed by tetrakis(acetonitrile)copper(I) hexafluorophosphate (2.50 eq, 57.8 mg, 0.155 mmol). The resulting colourless solution was capped and stirred at room temperature for 10 min (slowly turned green). The reaction was diluted with acetic acid, filtered, and purified via preparatory HPLC (15-65% acetonitrile in water with 0.1% TFA). Fractions containing the desired product were combined and lyophilized to dryness to afford perfluorophenyl 1-(4-(13-hydroxy-2,5,8,11-tetraoxatridecyl)-1H-1,2,3-triazol-1-yl)-3,6,9,12-tetraoxapentadecan-15-oate (Cpd. No. 26530) as a colourless liquid. Yield: 16.2 mg, 38%; LCMS m/z 690.5 [M+1]+; ¹H NMR (300 MHz, Chloroform-d) δ 7.97 (s, 1H), 4.81-4.72 (m, 2H), 4.64-4.54 (m, 2H), 3.96-3.82 (m, 4H), 3.80-3.56 (m, 29H), 2.94 (t, J=6.1 Hz, 2H).

### perfluorophenyl (RS)-9,14,17-trioxo-1-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)-16-(4-(3-(2-(2-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)ethoxy)ethoxy)propanamido)butyl)-3,6,21,24,27,30-hexaoxa-10,15,18-triazatritriacontan-33-oate (Cpd. No. 26531)

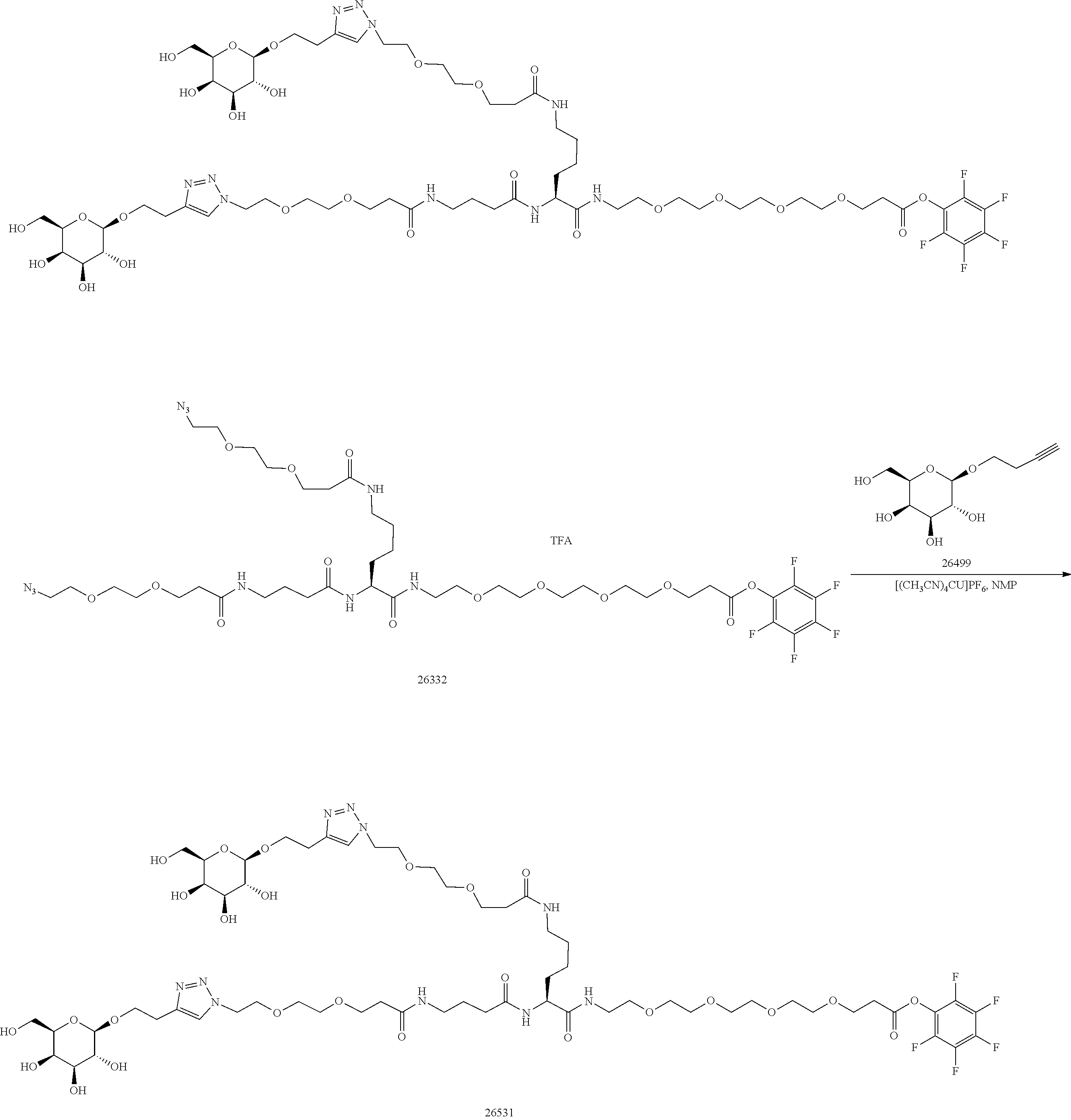

### Synthesis of perfluorophenyl (RS)-9,14,17-trioxo-1-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)-16-(4-(3-(2-(2-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)ethoxy)ethoxy)propanamido)butyl)-3,6,21,24,27,30-hexaoxa-10,15,18-triazatritriacontan-33-oate (Cpd. No. 26531)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 32.4 mg, 0.0287 mmol) and (2R,3R,4S,5R,6R)-2-(but-3-yn-1-yloxy)-6-(hydroxymethyl)tetrahydro-2H-pyran-3,4,5-triol (26499, 2.10 eq, 14.0 mg, 0.0603 mmol) in NMP (0.4 mL) in a 1 dram vial with a stirbar was added tetrakis(acetonitrile)copper(I) hexafluorophosphate (5.00 eq, 53.5 mg, 0.143 mmol). The resulting clear yellow solution was capped and stirred at room temperature for 10 min (slowly turned green). The reaction was diluted with a mixture of 70% acetic acid in NMP, filtered, and purified via preparatory HPLC (15-60% acetonitrile in water with 0.1% TFA). Fractions containing the desired product were combined and lyophilized to dryness to afford perfluorophenyl (RS)-9,14,17-trioxo-1-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)-16-(4-(3-(2-(2-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)ethoxy)ethoxy)propanamido)butyl)-3,6,21,24,27,30-hexaoxa-10,15,18-triazatritriacontan-33-oate (Cpd. No. 26531) as a white solid. Yield: 28.1 mg, 66%; LCMS m/z 1480.0 [M+1]+; ¹H NMR (300 MHz, DMSO-d₆ with D₂O) δ 7.85 (s, 2H), 4.45-4.35 (m, 3H), 4.21-4.10 (m, 2H), 3.98-2.70 (m, 62H), 2.31-2.19 (m, 4H), 2.09 (t, J=7.5 Hz, 2H), 1.64-1.37 (m, 4H), 1.37-1.07 (m, 4H).

### perfluorophenyl (R)-1-(4-(13-hydroxy-2,5,8,11-tetraoxatridecyl)-1H-1,2,3-triazol-1-yl)-16-(4-(3-(2-(2-(4-(13-hydroxy-2,5,8,11-tetraoxatridecyl)-1H-1,2,3-triazol-1-yl)ethoxy)ethoxy)propanamido)butyl)-9,14,17-trioxo-3,6,21,24,27,30-hexaoxa-10,15,18-triazatritriacontan-33-oate (Cpd. No. 26532)

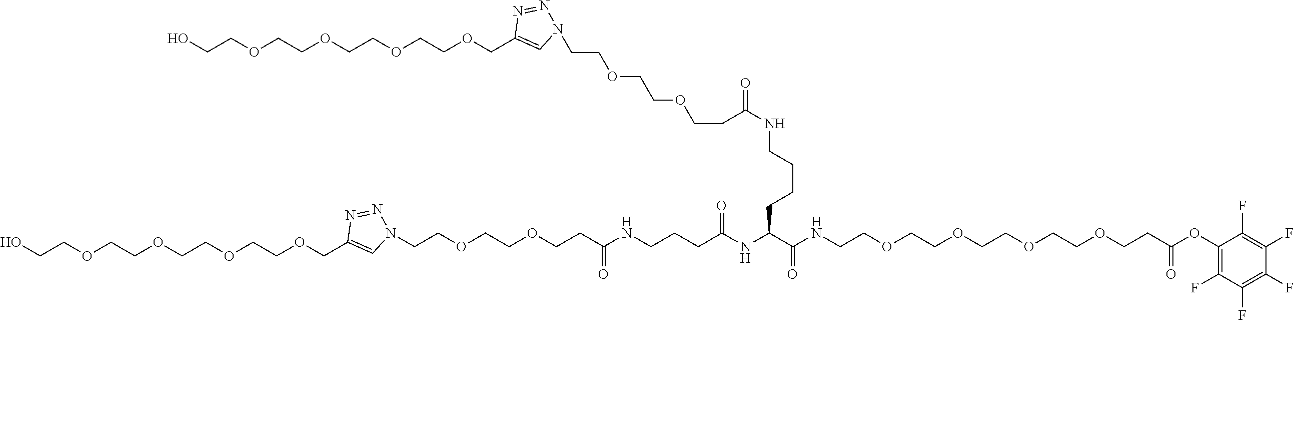 [SMILES: O=C(CCOCCOCCn1cc(COCCOCCOCCOCCO)nn1)NCCCC[C@H](NC(=O)CCCNC(=O)CCOCCOCCn1cc(COCCOCCOCCOCCO)nn1)C(=O)NCCOCCOCCOCCOCCC(=O)Oc1c(F)c(F)c(F)c(F)c1F]

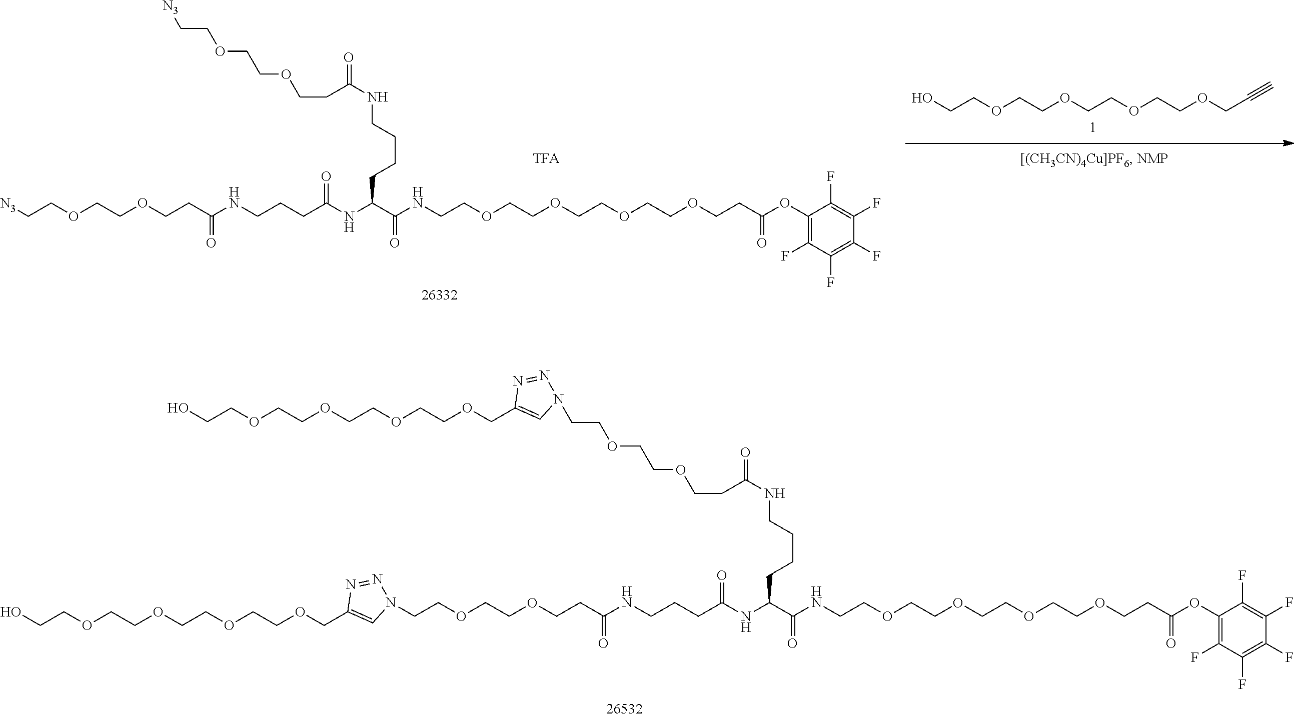 [SMILES: [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)Oc1c(F)c(F)c(F)c(F)c1F ; C#CCOCCOCCOCCOCCO ; O=C(CCOCCOCCn1cc(COCCOCCOCCOCCO)nn1)NCCCC[C@H](NC(=O)CCCNC(=O)CCOCCOCCn1cc(COCCOCCOCCOCCO)nn1)C(=O)NCCOCCOCCOCCOCCC(=O)Oc1c(F)c(F)c(F)c(F)c1F]

### Synthesis of perfluorophenyl (R)-1-(4-(13-hydroxy-2,5,8,11-tetraoxatridecyl)-1H-1,2,3-triazol-1-yl)-16-(4-(3-(2-(2-(4-(13-hydroxy-2,5,8,11-tetraoxatridecyl)-1H-1,2,3-triazol-1-yl)ethoxy)ethoxy)propanamido)butyl)-9,14,17-trioxo-3,6,21,24,27,30-hexaoxa-10,15,18-triazatritriacontan-33-oate (Cpd. No. 26532)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 30.2 mg, 0.0267 mmol) and 3,6,9,12-tetraoxapentadec-14-yn-1-ol (1, 2.10 eq, 13.0 mg, 0.0562 mmol) in NMP (0.5 mL) in a 1 dram vial with a stirbar was added tetrakis(acetonitrile)copper(I) hexafluorophosphate (5.00 eq, 49.8 mg, 0.134 mmol). The resulting clear yellow solution was capped and stirred at room temperature for 10 min (slowly turned green). The reaction was diluted with a mixture of 70% acetic acid in NMP, filtered, and purified via preparatory HPLC (15-60% acetonitrile in water with 0.1% TFA). Fractions containing the desired product were combined and lyophilized to dryness to afford perfluorophenyl (R)-1-(4-(13-hydroxy-2,5,8,11-tetraoxatridecyl)-1H-1,2,3-triazol-1-yl)-16-(4-(3-(2-(2-(4-(13-hydroxy-2,5,8,11-tetraoxatridecyl)-1H-1,2,3-triazol-1-yl)ethoxy)ethoxy)propanamido)butyl)-9,14,17-trioxo-3,6,21,24,27,30-hexaoxa-10,15,18-triazatritriacontan-33-oate (Cpd. No. 26532) as a clear yellow liquid. Yield: 17.0 mg, 43%; LCMS m/z 1480.1 [M+1]+; ¹H NMR (300 MHz, DMSO-d₆ with D₂O) δ 7.98 (s, 2H), 7.95-7.82 (m, 3H), 4.50-4.41 (m, 8H), 3.80-2.72 (m, 73H), 2.31-2.21 (m, 4H), 2.10 (t, J=7.4 Hz, 2H), 1.65-1.38 (m, 4H), 1.38-1.08 (m, 4H).

### perfluorophenyl (16RS,19RS)-9,14,17,20-tetraoxo-1-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)-16,19-bis(4-(3-(2-(2-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)ethoxy)ethoxy)propanamido)butyl)-3,6,24,27,30,33-hexaoxa-10,15,18,21-tetraazahexatriacontan-36-oate (Cpd. No. 26533)

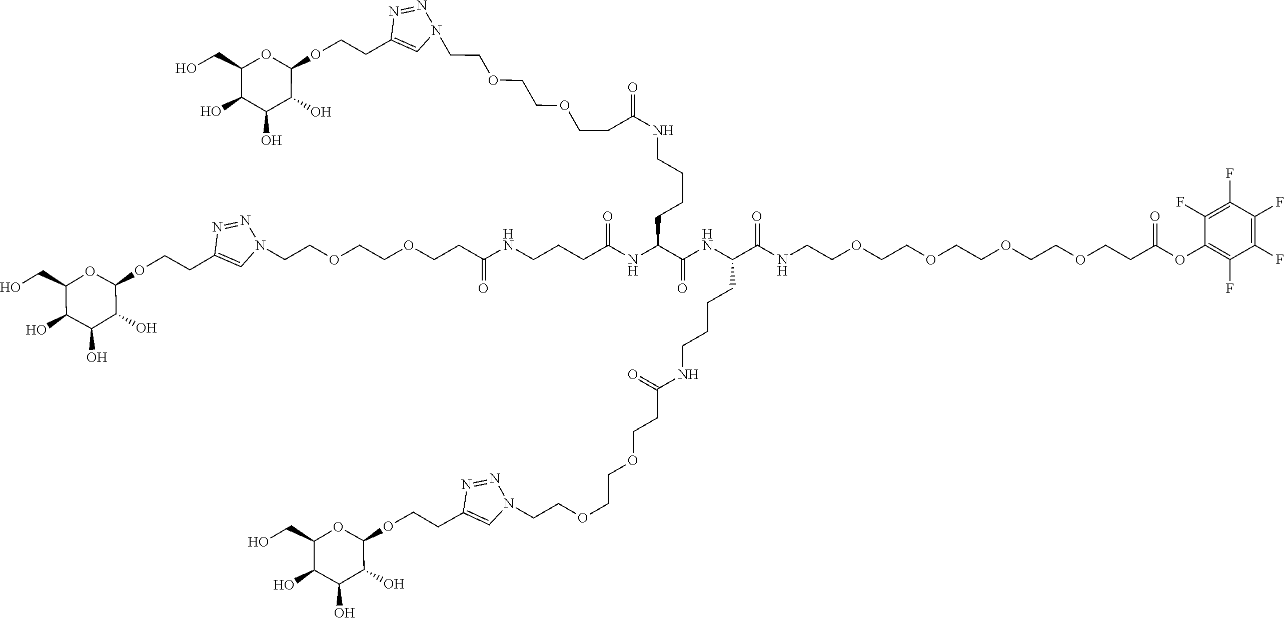 [SMILES: O=C(CCOCCOCCn1cc(CCO[C@@H]2O[C@H](CO)[C@H](O)[C@H](O)[C@H]2O)nn1)NCCCC[C@H](NC(=O)CCCNC(=O)CCOCCOCCn1cc(CCO[C@@H]2O[C@H](CO)[C@H](O)[C@H](O)[C@H]2O)nn1)C(=O)N[C@@H](CCCCNC(=O)CCOCCOCCn1cc(CCO[C@@H]2O[C@H](CO)[C@H](O)[C@H](O)[C@H]2O)nn1)C(=O)NCCOCCOCCOCCOCCC(=O)Oc1c(F)c(F)c(F)c(F)c1F]

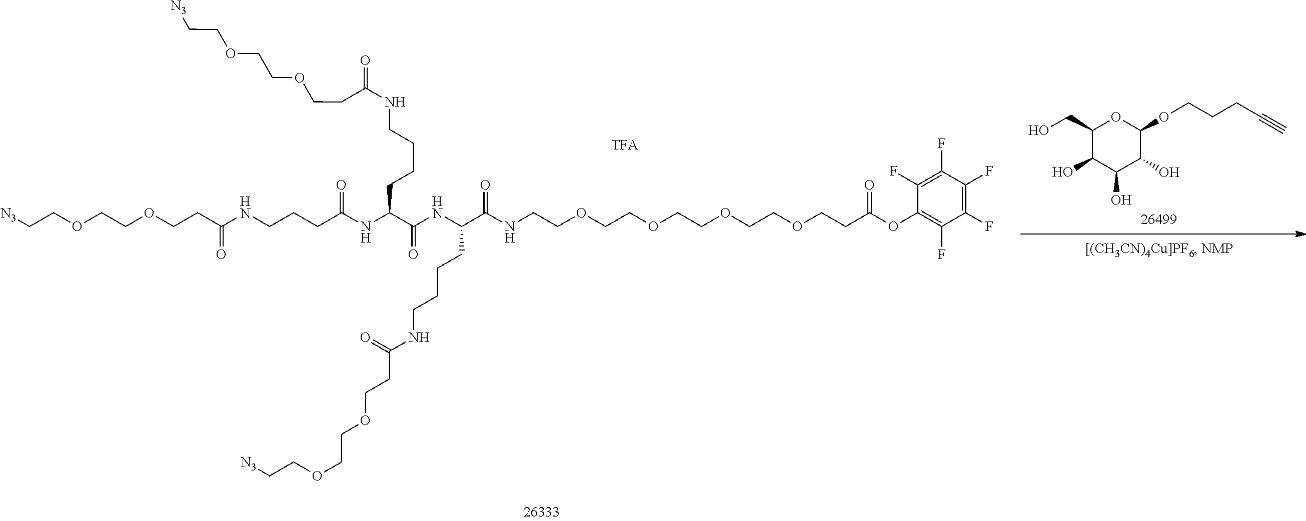 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F ; C#CCCCO[C@@H]1O[C@H](CO)[C@H](O)[C@H](O)[C@H]1O]

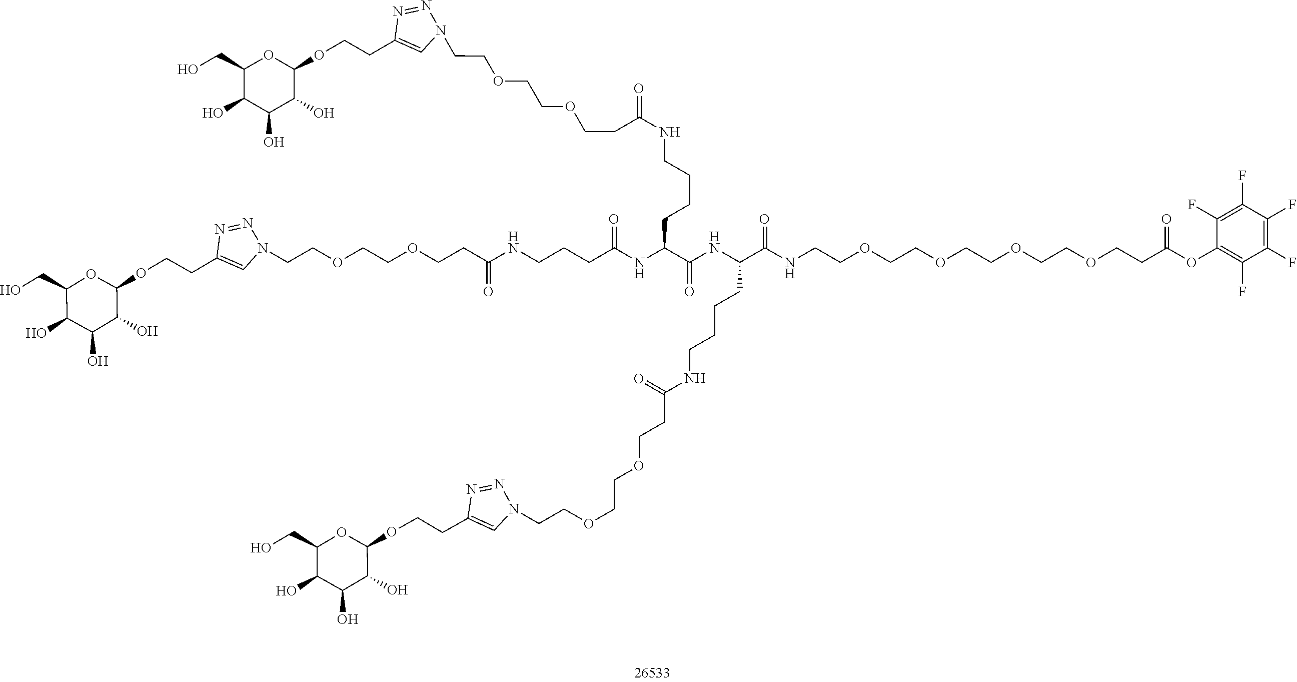 [SMILES: O=C(CCOCCOCCn1cc(CCO[C@@H]2O[C@H](CO)[C@H](O)[C@H](O)[C@H]2O)nn1)NCCCC[C@H](NC(=O)CCCNC(=O)CCOCCOCCn1cc(CCO[C@@H]2O[C@H](CO)[C@H](O)[C@H](O)[C@H]2O)nn1)C(=O)N[C@@H](CCCCNC(=O)CCOCCOCCn1cc(CCO[C@@H]2O[C@H](CO)[C@H](O)[C@H](O)[C@H]2O)nn1)C(=O)NCCOCCOCCOCCOCCC(=O)Oc1c(F)c(F)c(F)c(F)c1F]

### Synthesis of perfluorophenyl (16RS,19RS)-9,14,17,20-tetraoxo-1-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)-16,19-bis(4-(3-(2-(2-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)ethoxy)ethoxy)propanamido)butyl)-3,6,24,27,30,33-hexaoxa-10,15,18,21-tetraazahexatriacontan-36-oate (Cpd. No. 26533)

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 36.9 mg, 0.0256 mmol) and (2R,3R,4S,5R,6R)-2-(but-3-yn-1-yloxy)-6-(hydroxymethyl)tetrahydro-2H-pyran-3,4,5-triol (26499, 3.10 eq, 18.4 mg, 0.0793 mmol) in NMP (0.4 mL) in a 1 dram vial with a stirbar was added tetrakis(acetonitrile)copper(I) hexafluorophosphate (7.50 eq, 71.5 mg, 0.192 mmol). The resulting clear yellow solution was capped and stirred at room temperature for 10 min (slowly turned green). The reaction was diluted with a mixture of 70% acetic acid in NMP, filtered, and purified via preparatory HPLC (15-50% acetonitrile in water with 0.1% TFA). Fractions containing the desired product were combined and lyophilized to dryness to afford perfluorophenyl (16RS,19RS)-9,14,17,20-tetraoxo-1-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)-16,19-bis(4-(3-(2-(2-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)ethoxy)ethoxy)propanamido)butyl)-3,6,24,27,30,33-hexaoxa-10,15,18,21-tetraazahexatriacontan-36-oate (Cpd. No. 26533) as a white solid. Yield: 38.1 mg, 74%; LCMS m/z 1013.8 [M/2+1]+; ¹H NMR (300 MHz, DMSO-d₆ with D₂O) δ 7.85 (s, 3H), 4.45-4.36 (m, 6H), 4.18-2.65 (m, 83H), 2.31-2.16 (m, 6H), 2.16-2.04 (m, 2H), 1.66-1.38 (m, 6H), 1.38-1.06 (m, 10H).

### perfluorophenyl (16R,19R)-1-(4-(13-hydroxy-2,5,8,11-tetraoxatridecyl)-1H-1,2,3-triazol-1-yl)-16,19-bis(4-(3-(2-(2-(4-(13-hydroxy-2,5,8,11-tetraoxatridecyl)-1H-1,2,3-triazol-1-yl)ethoxy)ethoxy)propanamido)butyl)-9,14,17,20-tetraoxo-3,6,24,27,30,33-hexaoxa-10,15,18,21-tetraazahexatriacontan-36-oate (Cpd. No. 26534)

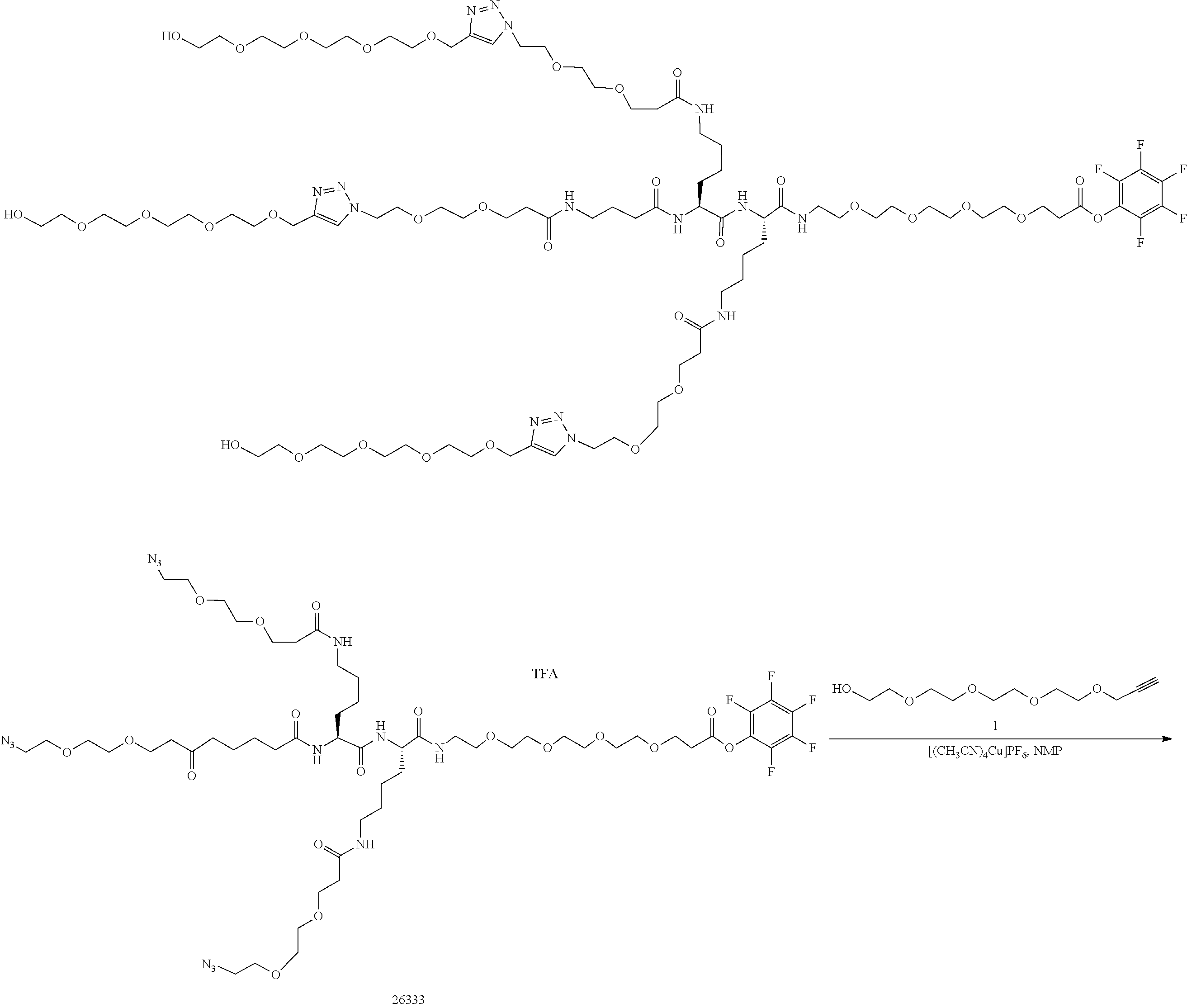 [SMILES: O=C(CCOCCOCCn1cc(COCCOCCOCCOCCO)nn1)NCCCC[C@H](NC(=O)CCCNC(=O)CCOCCOCCn1cc(COCCOCCOCCOCCO)nn1)C(=O)N[C@@H](CCCCNC(=O)CCOCCOCCn1cc(COCCOCCOCCOCCO)nn1)C(=O)NCCOCCOCCOCCOCCC(=O)Oc1c(F)c(F)c(F)c(F)c1F ; [N-]=[N+]=NCCOCCOCCC(=O)CCCCC(=O)N[C@@H](CCCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F ; C#CCOCCOCCOCCOCCO]

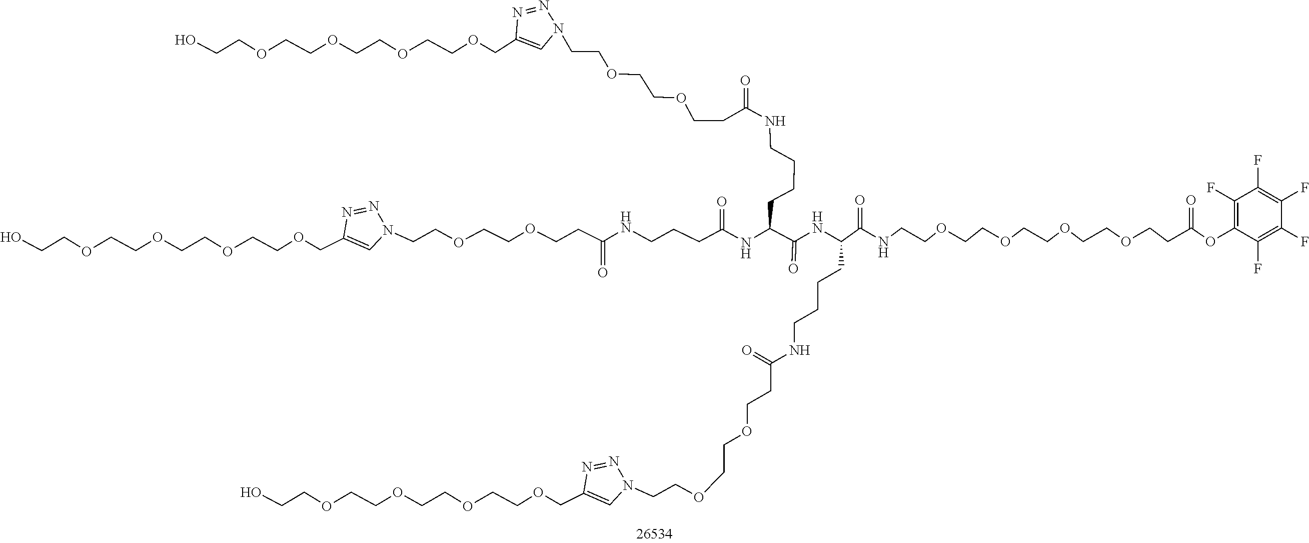 [SMILES: O=C(CCOCCOCCn1cc(COCCOCCOCCOCCO)nn1)NCCCC[C@H](NC(=O)CCCNC(=O)CCOCCOCCn1cc(COCCOCCOCCOCCO)nn1)C(=O)N[C@@H](CCCCNC(=O)CCOCCOCCn1cc(COCCOCCOCCOCCO)nn1)C(=O)NCCOCCOCCOCCOCCC(=O)Oc1c(F)c(F)c(F)c(F)c1F]

### Synthesis of perfluorophenyl (16R,19R)-1-(4-(13-hydroxy-2,5,8,11-tetraoxatridecyl)-1H-1,2,3-triazol-1-yl)-16,19-bis(4-(3-(2-(2-(4-(13-hydroxy-2,5,8,11-tetraoxatridecyl)-1H-1,2,3-triazol-1-yl)ethoxy)ethoxy)propanamido)butyl)-9,14,17,20-tetraoxo-3,6,24,27,30,33-hexaoxa-10,15,18,21-tetraazahexatriacontan-36-oate (Cpd. No. 26534)

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 35.8 mg, 0.0248 mmol) and 3,6,9,12-tetraoxapentadec-14-yn-1-ol (1, 3.10 eq, 17.9 mg, 0.0769 mmol) in NMP (0.4 mL) in a 1 dram vial with a stirbar was added tetrakis(acetonitrile)copper(I) hexafluorophosphate (7.50 eq, 69.4 mg, 0.186 mmol). The resulting clear green solution was capped and stirred at room temperature for 10 min. The reaction was diluted with a mixture of 70% acetic acid in NMP, filtered, and purified via preparatory HPLC (15-50% acetonitrile in water with 0.1% TFA). Fractions containing the desired product were combined and lyophilized to dryness to afford perfluorophenyl (16R,19R)-1-(4-(13-hydroxy-2,5,8,11-tetraoxatridecyl)-1H-1,2,3-triazol-1-yl)-16,19-bis(4-(3-(2-(2-(4-(13-hydroxy-2,5,8,11-tetraoxatridecyl)-1H-1,2,3-triazol-1-yl)ethoxy)ethoxy)propanamido)butyl)-9,14,17,20-tetraoxo-3,6,24,27,30,33-hexaoxa-10,15,18,21-tetraazahexatriacontan-36-oate (Cpd. No. 26534) as a clear pink liquid. Yield: 22.8 mg, 45%; LCMS m/z 1013.8 [M/2+1]+; ¹H NMR (300 MHz, DMSO-d₆ with D₂O) δ 7.98 (s, 3H), 7.95-7.83 (m, 2H), 4.51-4.39 (m, 12H), 3.80-2.69 (m, 100H), 2.31-2.19 (m, 6H), 2.15-2.04 (m, 2H), 1.67-1.38 (m, 6H), 1.38-1.07 (m, 8H).

### perfluorophenyl 1-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)-3,6,9,12-tetraoxapentadecan-15-oate (Cpd. No. 26535)

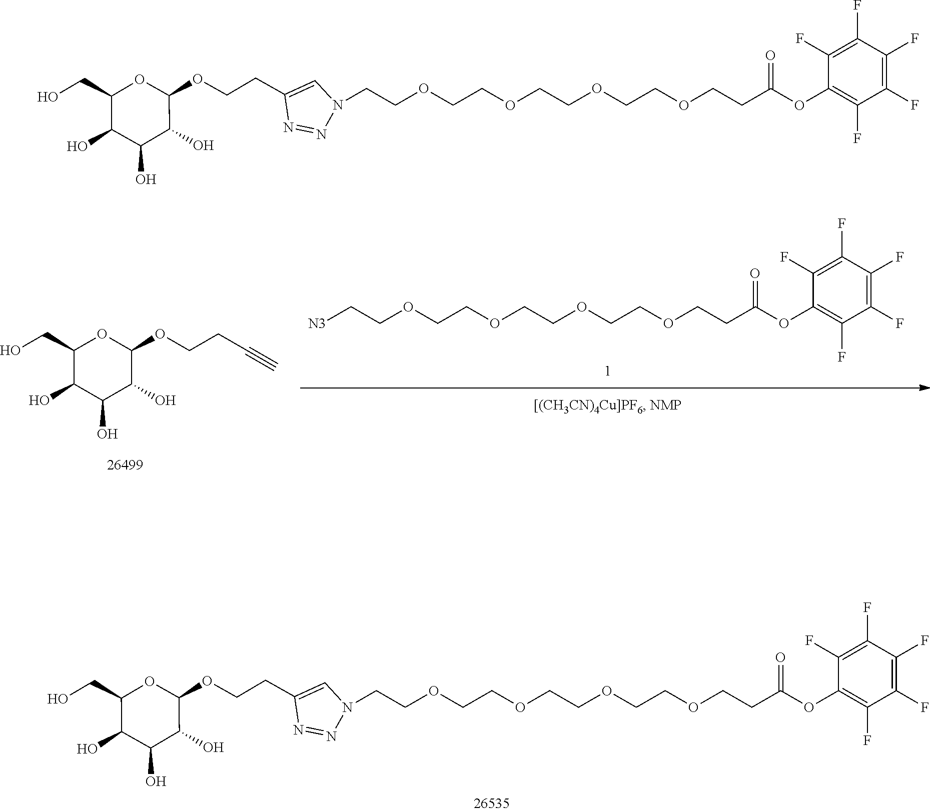 [SMILES: O=C(CCOCCOCCOCCOCCn1cc(CCO[C@@H]2O[C@H](CO)[C@H](O)[C@H](O)[C@H]2O)nn1)Oc1c(F)c(F)c(F)c(F)c1F ; C#CCCO[C@@H]1O[C@H](CO)[C@H](O)[C@H](O)[C@H]1O ; [N-]=[N+]=NCCOCCOCCOCCOCCC(=O)Oc1c(F)c(F)c(F)c(F)c1F ; O=C(CCOCCOCCOCCOCCn1cc(CCO[C@@H]2O[C@H](CO)[C@H](O)[C@H](O)[C@H]2O)nn1)Oc1c(F)c(F)c(F)c(F)c1F]

### Synthesis of perfluorophenyl 1-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)-3,6,9,12-tetraoxapentadecan-15-oate (Cpd. No. 26535)

To (2R,3R,4S,5R,6R)-2-(but-3-yn-1-yloxy)-6-(hydroxymethyl)tetrahydro-2H-pyran-3,4,5-triol (26499, 1.00 eq, 13.6 mg, 0.0586 mmol) in a 1 dram vial with a stirbar was added a solution of perfluorophenyl 1-azido-3,6,9,12-tetraoxapentadecan-15-oate (1, 1.10 eq, 29.5 mg, 0.0645 mmol) in NMP (0.5 mL) followed bytetrakis(acetonitrile)copper(I) hexafluorophosphate (2.50 eq, 54.6 mg, 0.147 mmol). The resulting clear yellow solution was capped and stirred at room temperature for 10 min (slowly turned green). The reaction was diluted with acetic acid, filtered, and purified via preparatory HPLC (15-65% acetonitrile in water with 0.1% TFA). Fractions containing the desired product were combined and lyophilized to dryness to afford perfluorophenyl 1-(4-(2-(((2R,3R,4S,5R,6R)-3,4,5-trihydroxy-6-(hydroxymethyl)tetrahydro-2H-pyran-2-yl)oxy)ethyl)-1H-1,2,3-triazol-1-yl)-3,6,9,12-tetraoxapentadecan-15-oate (Cpd. No. 26535) as a colourless semi solid. Yield: 29.0 mg, 72%; LCMS m/z [M+1]+; LCMS m/z 690.5 [M+1]+; ¹H NMR (300 MHz, DMSO-d₆ with D₂O) δ 7.88 (s, 1H), 4.47-4.37 (m, 2H), 4.16-4.09 (m, 1H), 3.80-3.21 (m, 24H), 2.95 (t, J=5.9 Hz, 2H), 2.86 (t, J=6.9 Hz, 2H).

### (1R,2R)-1-((2R,3R,4S,6R)-3-acetamido-4-acetoxy-6-hydroxy-6-(methoxycarbonyl)tetrahydro-2H-pyran-2-yl)-3-(3-phenoxybenzamido)propane-1,2-diyl diacetate (Cpd. No. 26565)

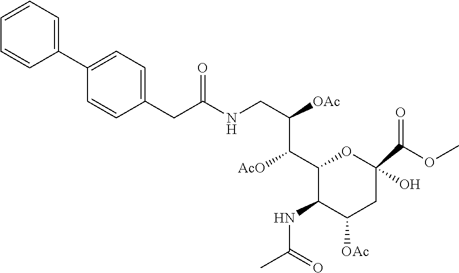 [SMILES: COC(=O)[C@@]1(O)C[C@H](OC(C)=O)[C@@H](NC(C)=O)[C@H]([C@H](OC(C)=O)[C@@H](CNC(=O)Cc2ccc(-c3ccccc3)cc2)OC(C)=O)O1]

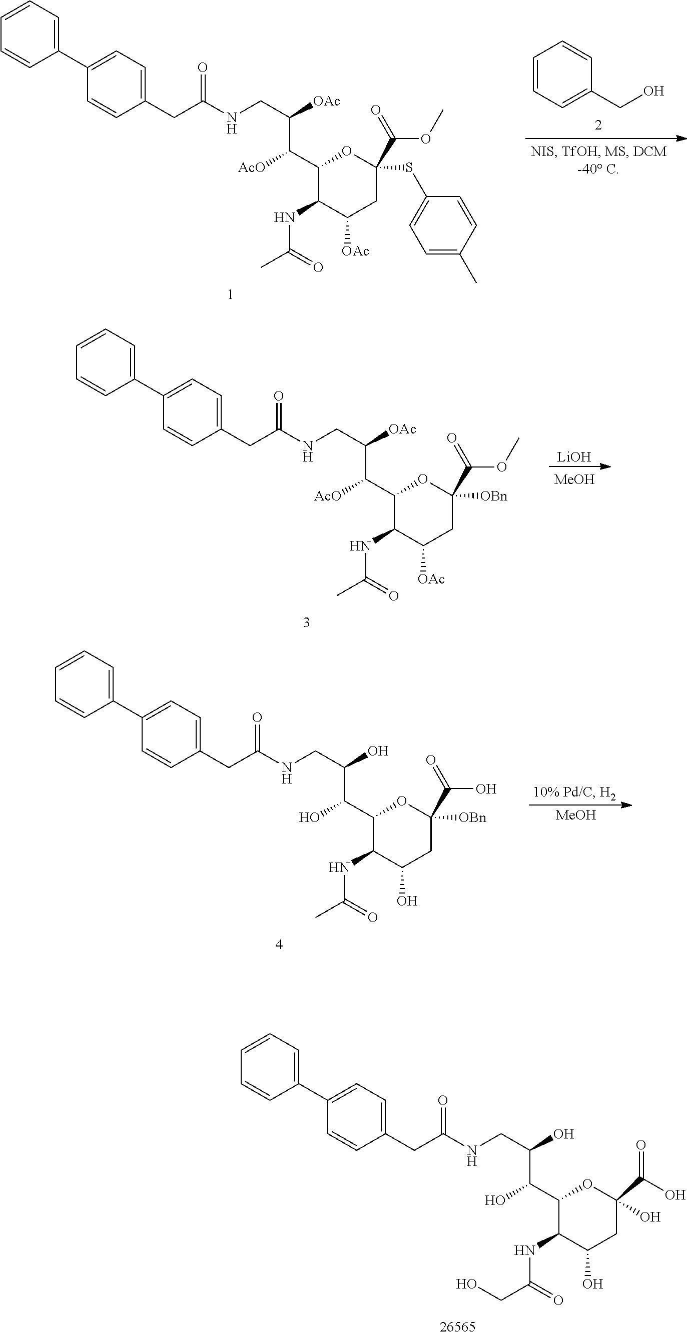

### Synthesis of (1R,2R)-3-(2-([1,1′-biphenyl]-4-yl)acetamido)-1-((2R,3R,4S,6R)-3-acetamido-4-acetoxy-6-(benzyloxy)-6-(methoxycarbonyl)tetrahydro-2H-pyran-2-yl)propane-1,2-diyl diacetate (3)

To a stirred solution of (1R,2R)-3-(2-([1,1′-biphenyl]-4-yl)acetamido)-1-((2R,3R,4S,6S)-3-acetamido-4-acetoxy-6-(methoxycarbonyl)-6-(p-tolylthio)tetrahydro-2H-pyran-2-yl)propane-1,2-diyl diacetate (1, 1.0 g, 1.24 mmol) in anhydrous dichloromethane (14.0 mL) was added phenylmethanol (0.386 mL, 3.72 mmol) and activated 4 Å powdered molecular sieves (1.0 g, 100% w/w). The resulting reaction mixture was stirred for 15 h under nitrogen atmosphere. The reaction mixture was cooled to −40° C. followed by the addition of 1-iodopyrrolidine-2,5-dione (0.697 g, 3.10 mmol) and trifluoromethanesulfonic acid (0.109 mL, 1.24 mmol) at −40° C. The reaction was stirred at −40° C. for 1 h. After completion, the reaction mixture was quenched with triethyl amine (0.1 mL, neutral pH) and warmed to room temperature. The reaction mixture was filtered and washed with dichloromethane. The filtrate was washed with a saturated solution of sodium bicarbonate and dried over sodium sulfate, filtered, and concentrated under reduced pressure to obtain a crude residue. The crude residue was purified via column chromatography (45-60% ethyl acetate in hexanes) to afford (1R,2R)-3-(2-([1,1′-biphenyl]-4-yl)acetamido)-1-((2R,3R,4S,6R)-3-acetamido-4-acetoxy-6-(benzyloxy)-6-(methoxycarbonyl)tetrahydro-2H-pyran-2-yl)propane-1,2-diyl diacetate (3) as an off white solid. Yield: 0.80 g, 81.30%; LCMS (ESI) m/z 791.82 [M+1]⁺.

### Synthesis of (2R,4S,5R,6R)-6-((1R,2R)-3-(2-([1,1′-biphenyl]-4-yl)acetamido)-1,2-dihydroxypropyl)-5-acetamido-2-(benzyloxy)-4-hydroxytetrahydro-2H-pyran-2-carboxylic acid (4)

To a stirred solution of (1R,2R)-3-(2-([1,1′-biphenyl]-4-yl)acetamido)-1-((2R,3R,4S,6R)-3-acetamido-4-acetoxy-6-(benzyloxy)-6-(methoxycarbonyl)tetrahydro-2H-pyran-2-yl)propane-1,2-diyl diacetate (3, 0.80 g, 1.01 mmol) in methanol (10.0 mL) at 0° C. was added lithium hydroxide (0.121 g, 5.06 mmol). The reaction mixture was allowed to warm to room temperature. After completion, Dowex-Hydrogen form was added to the reaction mass up to pH 6. The reaction mixture was filtered and the resulting filtrate was concentrated on a rotary evaporator to afford crude (2R,4S,5R,6R)-6-((1R,2R)-3-(2-([1,1′-biphenyl]-4-yl)acetamido)-1,2-dihydroxypropyl)-5-acetamido-2-(benzyloxy)-4-hydroxytetrahydro-2H-pyran-2-carboxylic acid (4) as a white solid. Yield: 0.60 g, 95.25%; LCMS (ESI) m/z 623.67 [M+1]⁺.

### Synthesis of (2R,4S,5R,6R)-6-((1R,2R)-3-(2-([1,1′-biphenyl]-4-yl)acetamido)-1,2-dihydroxypropyl)-2,4-dihydroxy-5-(2-hydroxyacetamido)tetrahydro-2H-pyran-2-carboxylic acid (Cpd. No. 26565)

To a stirred solution of crude (2R,4S,5R,6R)-6-((1R,2R)-3-(2-([1,1′-biphenyl]-4-yl)acetamido)-1,2-diacetoxypropyl)-5-acetamido-4-acetoxy-2-(benzyloxy)tetrahydro-2H-pyran-2-carboxylic acid (4, 0.60 g, 0.985 mmol) in methanol (10.0 mL) was added 10% Pd/C (0.30 g, 50% w/w) at room temperature. The reaction was then hydrogenated using a balloon pressure of H₂ gas for 12 h. After completion, the reaction was filtered through celite and the filtrate was concentrated. The obtained residue was purified via preparatory HPLC (19-38% acetonitrile in water with 0.1% TFA). Fractions containing the desired product were combined and lyophilized to dryness to afford (2R,4S,5R,6R)-6-((1R,2R)-3-(2-([1,1′-biphenyl]-4-yl)acetamido)-1,2-dihydroxypropyl)-2,4-dihydroxy-5-(2-hydroxyacetamido)tetrahydro-2H-pyran-2-carboxylic acid (Cpd. No. 26565) as the TFA salt as a white solid. Yield: 0.088 g, 17.22%; LCMS (ESI) m/z 517.34 [M+1]⁺; ¹H NMR (400 MHz, Methanol-d₄) δ 7.60-7.56 (m, 4H), 7.43-7.36 (m, 4H), 7.33-7.29 (m, 1H), 4.16-4.09 (m, 2H), 4.02 (s, 2H), 3.90 (t, J=10.4 Hz, 1H), 3.77-3.73 (m, 1H) 3.68-3.64 (m, 1H), 3.59 (m, 2H), 3.39 (d, J=9.2, 1H), 3.27-3.22 (m, 1H), 2.21 (dd, J=12.8 & 4.8 Hz, 1H), 1.84 1.83 (t, J=12.8 Hz, 1H).

### (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (Cpd. No. 26566)

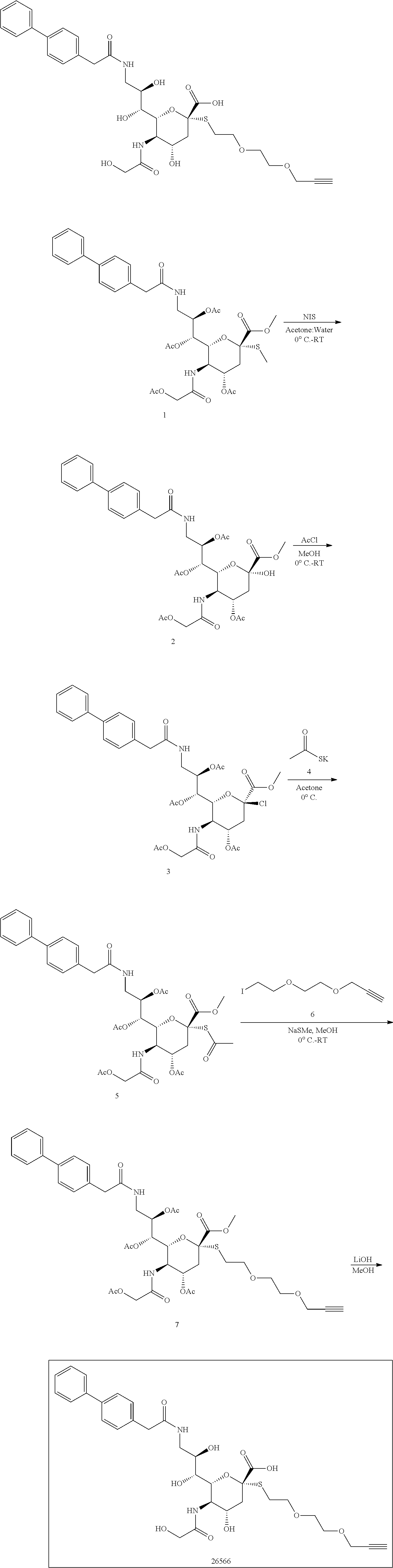

### Synthesis of (1R,2R)-3-(2-([1,1′-biphenyl]-4-yl)acetamido)-1-((2R,3R,4S,6R)-4-acetoxy-3-(2-acetoxyacetamido)-6-hydroxy-6-(methoxycarbonyl)tetrahydro-2H-pyran-2-yl)propane-1,2-diyl diacetate (2)

A stirred solution of (1R,2R)-3-(2-([1,1′-biphenyl]-4-yl)acetamido)-1-((2R,3R,4S,6S)-4-acetoxy-3-(2-acetoxyacetamido)-6-(methoxycarbonyl)-6-(p-tolylthio)tetrahydro-2H-pyran-2-yl)propane-1,2-diyl diacetate (1, 1.0 g, 1.24 mmol) in acetone:water (9:1, 20.0 mL) was cooled to 0° C. To the solution was added N-iodosuccinimide (0.35 g, 3.10 mmol) at 0° C. The resulting reaction solution was stirred at 0° C. for 3 h. After completion, a saturated aqueous solution of sodium metabisulfide (10.0 mL) and ethyl acetate (20.0 mL) was added. The reaction mixture was stirred for 10 min and transferred to a separatory funnel. The organic layer was separated and the aqueous phase was extracted with ethyl acetate (10.0 mL). The organic layers were combined and washed sequentially with saturated sodium bicarbonate solution and DM water. The organic layer was dried over anhydrous sodium sulfate and concentrated on a rotary evaporator to obtain a thick syrup. The thick syrup was purified via column chromatography (60-75% ethyl acetate in hexanes) to afford (1R,2R)-3-(2-([1,1′-biphenyl]-4-yl)acetamido)-1-((2R,3R,4S,6R)-4-acetoxy-3-(2-acetoxyacetamido)-6-hydroxy-6-(methoxycarbonyl)tetrahydro-2H-pyran-2-yl)propane-1,2-diyl diacetate (2) as a white solid. Yield: 0.76 g, 87.52%; LCMS (ESI) m/z 701.59 [M+1]⁺.

### Synthesis of (1R,2R)-3-(2-([1,1′-biphenyl]-4-yl)acetamido)-1-((2R,3R,4S,6R)-4-acetoxy-3-(2-acetoxyacetamido)-6-chloro-6-(methoxycarbonyl)tetrahydro-2H-pyran-2-yl)propane-1,2-diyl diacetate (3)

To a stirred solution of (1R,2R)-3-(2-([1,1′-biphenyl]-4-yl)acetamido)-1-((2R,3R,4S,6R)-4-acetoxy-3-(2-acetoxyacetamido)-6-hydroxy-6-(methoxycarbonyl)tetrahydro-2H-pyran-2-yl)propane-1,2-diyl diacetate (2, 0.76 g, 1.08 mmol) in acetyl chloride (30.0 mL) was added anhydrous methanol (0.3 mL) dropwise at 0° C. The resulting reaction mixture was stirred at room temperature for 24 h. After completion, the reaction mixture was concentrated under reduced pressure to afford crude (1R,2R)-3-(2-([1,1′-biphenyl]-4-yl)acetamido)-1-((2R,3R,4S,6R)-4-acetoxy-3-(2-acetoxyacetamido)-6-chloro-6-(methoxycarbonyl)tetrahydro-2H-pyran-2-yl)propane-1,2-diyl diacetate (3) as a light brown gel. Yield: 0.75 g, 97%; LC-MS (ESI) m/z 719.19 [M+1]⁺.

### Synthesis of (1R,2R)-3-(2-([1,1′-biphenyl]-4-yl)acetamido)-1-((2R,3R,4S,6S)-4-acetoxy-3-(2-acetoxyacetamido)-6-(acetylthio)-6-(methoxycarbonyl)tetrahydro-2H-pyran-2-yl)propane-1,2-diyl diacetate (5)

In an inert atmosphere, crude (1R,2R)-3-(2-([1,1′-biphenyl]-4-yl)acetamido)-1-((2R,3R,4S,6R)-4-acetoxy-3-(2-acetoxyacetamido)-6-chloro-6-(methoxycarbonyl)tetrahydro-2H-pyran-2-yl)propane-1,2-diyl diacetate (3, 0.75 g, 1.04 mmol, crude) was dissolved in dry acetone (10.0 mL) and stirred at 0° C. To this solution, potassium thioacetate (4, 0.357 g, 3.13 mmol) was added pinch-wise at 0° C. The reaction was stirred for 3 h at 0° C. After completion, the mixture was concentrated under reduced pressure to obtain a crude residue. The crude residue was dissolved in ethyl acetate and the resulting solution was washed with 1N HCl followed by DM water. The organic layer was separated, dried over anhydrous sodium sulfate, and concentrated on a rotary evaporator to obtain a thick residue. The residue was purified via column chromatography (60-70% ethyl acetate in hexanes) to afford (1R,2R)-3-(2-([1,1′-biphenyl]-4-yl)acetamido)-1-((2R,3R,4S,6S)-4-acetoxy-3-(2-acetoxyacetamido)-6-(acetylthio)-6-(methoxycarbonyl)tetrahydro-2H-pyran-2-yl)propane-1,2-diyl diacetate (5) as a white solid. Yield: 0.530 g, 66.97%; LCMS (ESI) m/z 759.29 [M+1]⁺.

### Synthesis of (1R,2R)-3-(2-([1,1′-biphenyl]-4-yl)acetamido)-1-((2R,3R,4S,6S)-4-acetoxy-3-(2-acetoxyacetamido)-6-(methoxycarbonyl)-6-((2-(2-(prop-2-yn-1-yloxy)ethoxy)ethyl)thio)tetrahydro-2H-pyran-2-yl)propane-1,2-diyl diacetate (Cpd. No. 26566)

To a stirred solution of (1R,2R)-3-(2-([1,1′-biphenyl]-4-yl)acetamido)-1-((2R,3R,4S,6S)-4-acetoxy-3-(2-acetoxyacetamido)-6-(acetylthio)-6-(methoxycarbonyl)tetrahydro-2H-pyran-2-yl)propane-1,2-diyl diacetate (5, 0.53 g, 0.698 mmol) in methanol (10.0 mL) was added sodium thiomethoxide (0.058 g, 0.838 mmol) at 0° C. The resulting reaction solution was stirred for 1 h. To the reaction solution was added 1-iodo-2-[2-(prop-2-yn-1-yloxy)ethoxy]ethane (0.354 g, 1.40 mmol) at 0° C. The resulting reaction mixture was stirred at room temperature for 1 h. After completion, lithium hydroxide (0.026 g, 1.12 mmol) was added at room temperature. The resulting reaction mixture was stirred at room temperature for 6 h. After completion, Dowex-hydrogen form was added up to pH 6 and the reaction mass was filtered through a sintered funnel. The filtrate was concentrated on a rotary evaporator to obtain a thick residue which was then purified via preparatory HPLC (15-40% acetonitrile in water with 0.1% TFA). Fractions containing the desired product were combined and lyophilized to dryness to afford (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (Cpd. No. 26566) as the TFA salt as a white solid. Yield: 0.170 g, 46.04%; LC-MS (ESI) m/z 661.17 [M+1]⁺. ¹H NMR (400 MHz, methanol-d₄) δ 7.61-7.57 (m, 4H), 7.44-7.37 (4H), 7.33-7.30 (m, 1H), 4.15 (d, J=2.0 Hz, 1H), 4.00 (s, 2H), 3.86-3.81 (m, 3H), 3.69-3.54 (m, 11H), 3.37-3.35 (m, 1H), 3.25-3.22 (m, 1H), 2.95-2.89 (m, 1H), 2.86-2.76 (m, 3H), 1.85-1.75 (m, 1H).

### (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (Cpd. No. 26567)

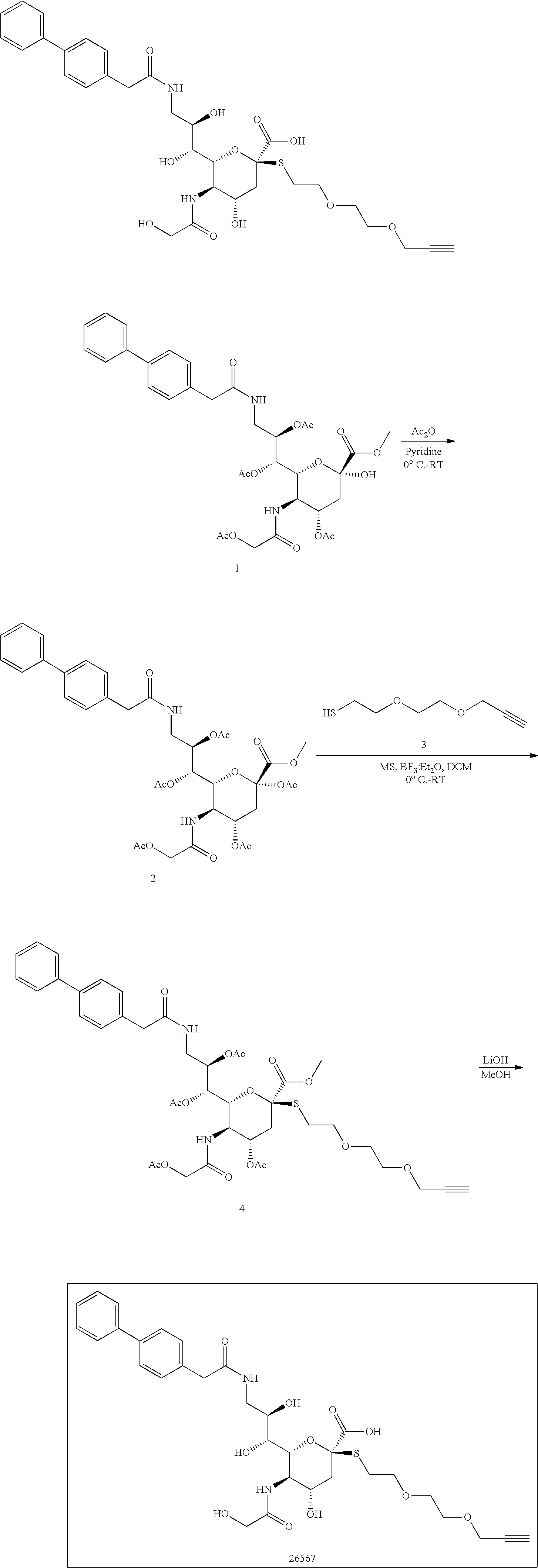

### Synthesis of (2S,4S,5R,6R)-6-((1R,2R)-3-(2-([1,1′-biphenyl]-4-yl)acetamido)-1,2-diacetoxypropyl)-5-(2-acetoxyacetamido)-2-(methoxycarbonyl)tetrahydro-2H-pyran-2,4-diyl diacetate (2)

To a stirred solution of (1R,2R)-3-(2-([1,1′-biphenyl]-4-yl)acetamido)-1-((2R,3R,4S,6R)-4-acetoxy-3-(2-acetoxyacetamido)-6-hydroxy-6-(methoxycarbonyl)tetrahydro-2H-pyran-2-yl)propane-1,2-diyl diacetate (1, 0.70 g, 0.99 mmol) in pyridine (10.0 mL) was dropwise added acetic anhydride (0.188 mL, 2.0 mmol) at 0° C. The reaction mixture was allowed to warm to room temperature and stirred overnight. After completion, volatiles were removed under vacuum to obtain a crude thick syrup. The thick syrup was poured into a separatory funnel with ethyl acetate (20.0 mL) and washed with 1N HCl solution followed by saturated sodium sulfate solution and DM water. The organic layer was dried over anhydrous sodium sulfate and concentrated under reduced pressure to obtain a crude thick syrup. The thick syrup was purified via column chromatography (55-70% ethyl acetate in hexanes) to afford (2S,4S,5R,6R)-6-((1R,2R)-3-(2-([1,1′-biphenyl]-4-yl)acetamido)-1,2-diacetoxypropyl)-5-(2-acetoxyacetamido)-2-(methoxycarbonyl)tetrahydro-2H-pyran-2,4-diyl diacetate (2) as a white solid. Yield: 0.50 g, 67.39%; LCMS (ESI) m/z 743.27 [M+1]⁺.

### Synthesis of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-((2-(2-(prop-2-yn-1-yloxy)ethoxy)ethyl)thio)tetrahydro-2H-pyran-2-yl)propane-1,2-diyl diacetate (4)

In an inert atmosphere, (2S,4S,5R,6R)-6-((1R,2R)-3-(2-([1,1′-biphenyl]-4-yl)acetamido)-1,2-diacetoxypropyl)-5-(2-acetoxyacetamido)-2-(methoxycarbonyl)tetrahydro-2H-pyran-2,4-diyl diacetate (2, 0.50 g, 0.673 mmol) and 2-(2-(prop-2-yn-1-yloxy)ethoxy)ethane-1-thiol (3, 0.215 g, 1.35 mmol) were dissolved in dry dichloromethane (10.0 mL) with stirring at room temperature. To the resulting reaction solution was added activated powdered 4 Å molecular sieves (0.50 g, 100% w/w) at room temperature. The resulting reaction mixture was stirred for 30 min. The reaction mixture was cooled to 0° C. and BF₃·Et₂O (0.586 mL, 2.02 mmol) was added drop-wise at 0° C. over 5 min. The resulting reaction mixture was stirred at room temperature for 16 h. After completion, the reaction mixture was quenched with triethylamine up to neutral pH. The reaction mixture was filtered and washed with an aqueous saturated sodium bicarbonate solution. The organic layer was separated, dried over anhydrous sodium sulfate, and concentrated on a rotary evaporator to obtain a crude thick syrup. The thick syrup was purified via column chromatography (60-70% ethyl acetate in hexanes) to afford (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-((2-(2-(prop-2-yn-1-yloxy)ethoxy)ethyl)thio)tetrahydro-2H-pyran-2-yl)propane-1,2-diyl diacetate (4) as a white solid. Yield: 0.30 g, 52.86%; LCMS (ESI) m/z 843.91 [M+1]⁺.

### Synthesis of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (Cpd. No. 26567)

To a stirred solution of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-((2-(2-(prop-2-yn-1-yloxy)ethoxy)ethyl)thio)tetrahydro-2H-pyran-2-yl)propane-1,2-diyl diacetate (4, 0.30 g, 0.355 mmol) in methanol (10.0 mL) at 0° C. was added lithium hydroxide (0.051 g, 2.14 mmol). The reaction mixture was allowed to warm to room temperature and stirred for 6 h. After completion, Dowex-Hydrogen form was added up to neutral pH and the reaction mixture was filtered through a sintered funnel. The filtrate was removed under vacuum to obtain a crude thick syrup which was purified via preparatory HPLC (22-48% acetonitrile in water with 0.1% TFA). Fractions containing the desired product were combined and lyophilized to dryness to afford (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (Cpd. No. 26567) as an amorphous solid. Yield: 0.113 g, 48.05%; LCMS (ESI) m/z 661.36 [M+1]⁺. ¹H NMR (400 MHz, methanol-d₄) δ 7.88 (d, J=8.8 Hz, 1H), 7.61-7.57 (m, 4H), 7.44-7.29 (m, 5H), 4.26 (d, J=10.4 Hz, 1H), 4.17-4.15 (m, 3H), 4.03 (s, 2H), 3.90-3.83 (m, 2H), 3.63-3.56 (m, 9H), 3.41-3.34 (m, 2H), 2.85-2.81 (m, 3H), 2.47 (dd, J=14.0, 4.8 Hz, 1H), 1.95 (t, J=14.0 Hz, 1H).

### (2R,4S,5R,6R)-4-hydroxy-5-(2-hydroxyacetamido)-2-(2-(2-(prop-2-yn-1-yloxy)ethoxy)ethoxy)-6-((1R,2R)-1,2,3-trihydroxypropyl)tetrahydro-2H-pyran-2-carboxylic acid (Cpd. No. 26591) and (2S,4S,5R,6R)-4-hydroxy-5-(2-hydroxyacetamido)-2-(2-(2-(prop-2-yn-1-yloxy)ethoxy)ethoxy)-6-((1R,2R)-1,2,3-trihydroxypropyl)tetrahydro-2H-pyran-2-carboxylic acid (Cpd. No. 26568)

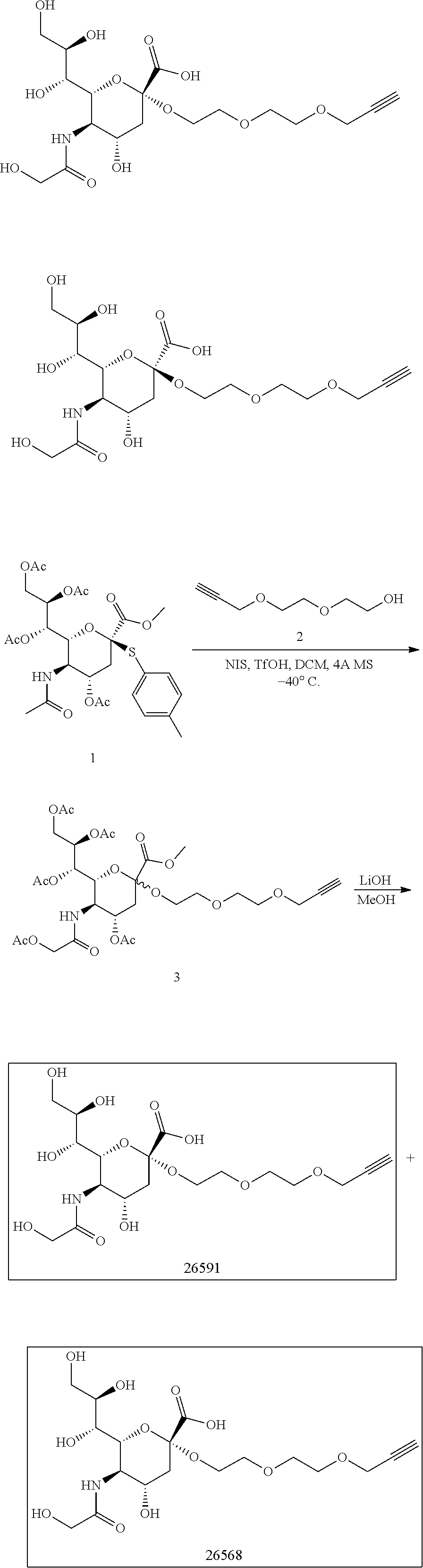

### Synthesis of (1S,2R)-1-((2R,3R,4S)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2,3-triyl triacetate (3)

To a stirred solution of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (1, 2.0 g, 3.34 mmol) in dichloromethane (32.0 mL) was added 2-(2-(prop-2-yn-1-yloxy)ethoxy)ethan-1-ol (2, 1.206 g, 8.36 mmol) and 4 Å MS (2.0 g 100% w/w). The resulting suspension was stirred under nitrogen atmosphere for 12 h. The reaction mixture was cooled to −40° C., followed by the sequential addition of N-iodosuccinimide (1.882 g, 8.36 mmol) and trifluoromethane sulfonic acid (0.294 mL, 3.34 mmol) dropwise at −40° C. The resulting reaction mixture was stirred at −40° C. for 1 h. After completion, triethylamine was added up to neutral pH and the reaction mixture was filtered through celite. The filtrate was concentrated under reduced pressure to obtain a crude residue which was then purified with column chromatography (40-60% ethyl acetate in hexanes) to afford (1S,2R)-1-((2R,3R,4S)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2,3-triyl triacetate (3) as a white solid as an anomeric mixture. Yield: 1.50 g, 66.34%; LC-MS (ESI) m/z 676.14 [M+1]⁺.

### Synthesis of (2R,4S,5R,6R)-4-hydroxy-5-(2-hydroxyacetamido)-2-(2-(2-(prop-2-yn-1-yloxy)ethoxy)ethoxy)-6-((1R,2R)-1,2,3-trihydroxypropyl)tetrahydro-2H-pyran-2-carboxylic acid (Cpd. No. 26591) and (2S,4S,5R,6R)-4-hydroxy-5-(2-hydroxyacetamido)-2-(2-(2-(prop-2-yn-1-yloxy)ethoxy)ethoxy)-6-((1R,2R)-1,2,3-trihydroxypropyl)tetrahydro-2H-pyran-2-carboxylic acid (Cpd. No. 26568)

To a stirred solution of (1S,2R)-1-((2R,3R,4S)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2,3-triyl triacetate (3, 1.50 g, 2.22 mmol) in methanol (20.0 mL) at 0° C. was added lithium hydroxide (0.264 g, 11.1 mmol). The reaction mixture was allowed to warm to room temperature and stirred for 6 h. After completion, Dowex-Hydrogen form was added up to neutral pH and the reaction mixture was filtered through a sintered funnel. The filtrate was removed under vacuum to obtain a crude thick syrup which was then purified via preparatory HPLC (15-45% acetonitrile in water with 0.1% TFA). Fractions containing the desired product (two peaks) were combined and lyophilized to dryness to afford (2R,4S,5R,6R)-4-hydroxy-5-(2-hydroxyacetamido)-2-(2-(2-(prop-2-yn-1-yloxy)ethoxy)ethoxy)-6-((1R,2R)-1,2,3-trihydroxypropyl)tetrahydro-2H-pyran-2-carboxylic acid (Cpd. No. 26591) as a white solid. Yield: 0.234 g, 46.70%; ELSD-MS (ESI) m/z 452.2 [M+1]⁺. ¹H NMR (400 MHz, methanol-d₄) δ 7.90 (d, J=7.6 Hz, 1H), 4.19 (d, J=2.4 Hz, 2H), 4.03 (s, 2H), 3.94-3.90 (m, 1H), 3.86-3.79 (m, 4H), 3.70-3.51 (m, 10H), 3.34-3.31 (m, 1H), 2.83 (t, J=2.4 Hz, 1H), 2.74 (dd, J=11.8 & 4.4 Hz, 1H), 1.95 (t, J=11.8 Hz, 1H); (2S,4S,5R,6R)-4-hydroxy-5-(2-hydroxyacetamido)-2-(2-(2-(prop-2-yn-1-yloxy)ethoxy)ethoxy)-6-((1R,2R)-1,2,3-trihydroxypropyl)tetrahydro-2H-pyran-2-carboxylic acid (Cpd. No. 26568) as a white solid. Yield: 0.268 g, 53.48%; ELSD-MS (ESI) m/z 452.2 [M+1]⁺. ¹H NMR (400 MHz, methanol-d₄) δ 7.81 (d, J=7.6 Hz, 1H), 4.20 (d, J=2.4 Hz, 2H), 4.19-4.15 (m, 1H), 4.04 (s, 2H), 4.02-4.00 (m, 1H), 3.95-3.91 (m, 1H), 3.69-3.62 (m, 7H), 3.53-3.47 (m, 2H), 2.84 (t, J=2.4 Hz, 1H), 2.39 (dd, J=12.8 & 4.8 Hz, 1H), 1.95 (t, J=12.0 Hz, 1H).

### Synthesis of (2S,4S,5R,6R)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)-6-((1R,2R)-1,2,3-trihydroxypropyl)tetrahydro-2H-pyran-2-carboxylic acid (Cpd. No. 26590)

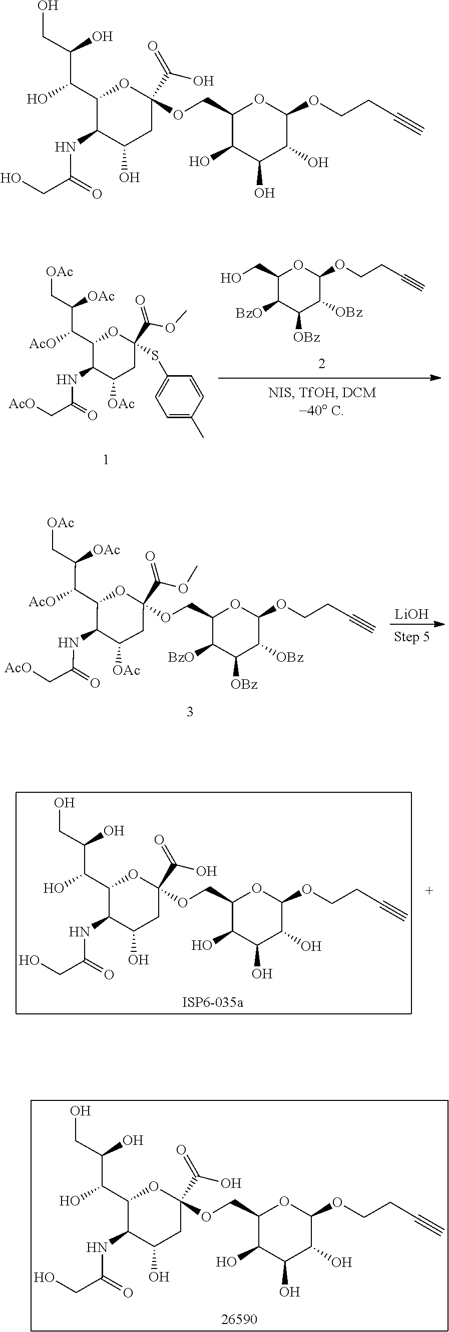

### Synthesis of (2R,3S,4S,5R,6R)-2-((((2R,4S,5R,6R)-4-acetoxy-5-(2-acetoxyacetamido)-2-(methoxycarbonyl)-6-((1S,2R)-1,2,3-triacetoxypropyl)tetrahydro-2H-pyran-2-yl)oxy)methyl)-6-(but-3-yn-1-yloxy)tetrahydro-2H-pyran-3,4,5-triyl tribenzoate (3)

To a stirred suspension of (1S,2R)-1-((2R,3R,4S,6S)-4-acetoxy-3-(2-acetoxyacetamido)-6-(methoxycarbonyl)-6-(p-tolylthio)tetrahydro-2H-pyran-2-yl)propane-1,2,3-triyl triacetate (1, 1.0 g, 1.53 mmol) in anhydrous dichloromethane (18.0 mL) was added (2R,3R,4S,5S,6R)-2-(but-3-yn-1-yloxy)-6-(hydroxymethyl)tetrahydro-2H-pyran-3,4,5-triyl tribenzoate (2, 2.08 g, 3.81 mmol) and activated 4 Å powdered molecular sieves (1.0 g, 100% w/w) at room temperature for 15 h under nitrogen atmosphere. The reaction mixture was cooled to −40° C. followed by the addition of 1-iodopyrrolidine-2,5-dione (0.823 g, 3.66 mmol) and trifluoromethanesulfonic acid (0.134 mL, 1.53 mmol) at −40° C. The reaction was stirred at −40° C. for 1 h. After completion, the reaction mixture was quenched by triethyl amine (0.1 mL, neutral pH) and warmed to room temperature. The reaction mixture was filtered through a sintered funnel and washed by dichloromethane. The filtrate was washed by a saturated solution of sodium bicarbonate and dried over sodium sulfate, filtered, and concentrated under reduced pressure to obtain a crude residue. The crude residue was purified via column chromatography (45-55% ethyl acetate in hexanes) to afford (2R,3S,4S,5R,6R)-2-((((2R,4S,5R,6R)-4-acetoxy-5-(2-acetoxyacetamido)-2-(methoxycarbonyl)-6-((1S,2R)-1,2,3-triacetoxypropyl)tetrahydro-2H-pyran-2-yl)oxy)methyl)-6-(but-3-yn-1-yloxy)tetrahydro-2H-pyran-3,4,5-triyl tribenzoate (3) as an off white solid. Yield: 0.60 g, 36.56%; LCMS (ESI) m/z 1077.02 [M+1]⁺.

### Synthesis of (2S,4S,5R,6R)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)-6-((1R,2R)-1,2,3-trihydroxypropyl)tetrahydro-2H-pyran-2-carboxylic acid (Cpd. No. 26590)

To a stirred solution of (2R,3S,4S,5R,6R)-2-((((2R,4S,5R,6R)-4-acetoxy-5-(2-acetoxyacetamido)-2-(methoxycarbonyl)-6-((1S,2R)-1,2,3-triacetoxypropyl)tetrahydro-2H-pyran-2-yl)oxy)methyl)-6-(but-3-yn-1-yloxy)tetrahydro-2H-pyran-3,4,5-triyl tribenzoate (3, 0.80 g, 1.01 mmol) in methanol (10.0 mL) at 0° C. was added lithium hydroxide (0.080 g, 3.35 mmol). The mixture was allowed to warm to room temperature and stirred for 6 h. After completion, Dowex-Hydrogen form was added to the reaction mass up to pH 6. The reaction mixture was filtered through a sintered funnel and the filtrate was removed under vacuum to obtain a crude thick syrup which was then purified via preparatory HPLC (20-45% acetonitrile in water with 0.1% TFA). Fractions containing the desired product were combined and lyophilized to dryness to afford (2S,4S,5R,6R)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)-6-((1R,2R)-1,2,3-trihydroxypropyl)tetrahydro-2H-pyran-2-carboxylic acid (Cpd. No. 26590) as the TFA salt as a white solid. Yield: 0.053 g, 17.62%; ELSD-MS (ESI) m/z 540.2 [M+1]⁺; ¹H NMR (400 MHz, methanol-d₄) δ 4.26 (d, J=7.2 Hz, 1H), 4.17-4.12 (m, 1H), 4.04-4.01 (m, 3H), 3.95-3.63 (m, 10H), 3.53-3.47 (m, 4H), 2.51 (dt, J=7.2 & 2.4 Hz, 2H), 2.40 (dd, J=13.2 & 5.2 Hz, 1H), 2.62 (t, J=2.4 Hz, 1H), 1.66 (t, J=12.8 Hz, 1H).

### Perfluorophenyl (S)-23-azido-18-(4-azidobutyl)-17,20-dioxo-4,7,10,13-tetraoxa-16,19-diazatricosanoate (Cpd. No. 26594)

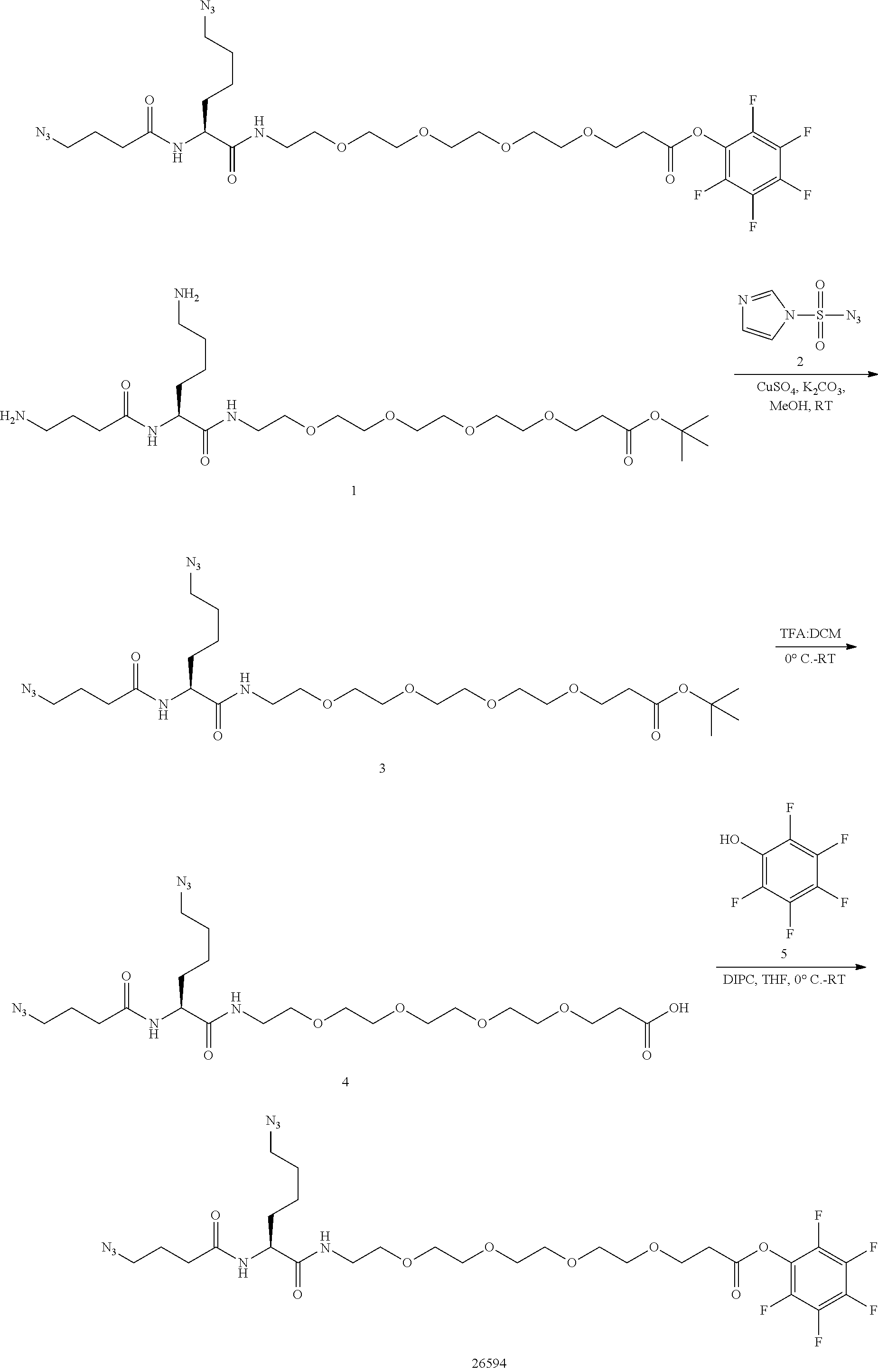

### Synthesis of tert-butyl (S)-23-azido-18-(4-azidobutyl)-17,20-dioxo-4,7,10,13-tetraoxa-16,19-diazatricosanoate (3)

To a stirred solution of tert-butyl (S)-23-amino-18-(4-aminobutyl)-17,20-dioxo-4,7,10,13-tetraoxa-16,19-diazatricosanoate (1, 3.0 g, 5.61 mmol) in methanol (60.0 mL) was added potassium carbonate (2.32 g, 16.85 mmol), copper sulfate (0.219 g, 1.40 mmol), and 1H-imidazole-1-sulfonyl azide hydrochloride (2, 2.57 g, 12.3 mmol). The resulting reaction mixture was stirred at room temperature for 24 h. After completion, the reaction mixture was diluted with 1N hydrochloric acid solution up to pH 3 and extracted with ethyl acetate. The organic layer was dried over sodium sulfate, filtered, and concentrated under high vacuum to obtain a crude residue. The crude residue was purified via flash column chromatography (0-5% methanol in dichloromethane) to afford tert-butyl (S)-23-azido-18-(4-azidobutyl)-17,20-dioxo-4,7,10,13-tetraoxa-16,19-diazatricosanoate (3) as a pale yellow viscous liquid. Yield: 1.50 g, 45%; ELSD m/z 587.3 [M+1]⁺.

### Synthesis of (S)-23-azido-18-(4-azidobutyl)-17,20-dioxo-4,7,10,13-tetraoxa-16,19-diazatricosanoic acid (4)

To a stirred solution of tert-butyl (S)-23-azido-18-(4-azidobutyl)-17,20-dioxo-4,7,10,13-tetraoxa-16,19-diazatricosanoate (3, 1.50 g, 2.55 mmol) in dichloromethane (20.0 mL) was added trifluoroacetic acid (5.0 mL) at 0° C. The resulting reaction mixture was stirred at room temperature under nitrogen for 4 h. After completion, the reaction mixture was concentrated and dried to afford (S)-23-azido-18-(4-azidobutyl)-17,20-dioxo-4,7,10,13-tetraoxa-16,19-diazatricosanoic acid (4) as a pale yellow viscous liquid. Yield: 1.30 g, 96%; ELSD m/z 531.3 [M+1]⁺.

### Synthesis of perfluorophenyl (S)-23-azido-18-(4-azidobutyl)-17,20-dioxo-4,7,10,13-tetraoxa-16,19-diazatricosanoate (Cpd. No. 26594)

To a stirred solution of S)-23-azido-18-(4-azidobutyl)-17,20-dioxo-4,7,10,13-tetraoxa-16,19-diazatricosanoic acid (4, 1.30 g, 2.45 mmol) in tetrahydrofuran (20.0 mL) at 0° C. was added 2,3,4,5,6-pentafluorophenol (5, 0.897 g, 4.90 mmol) and N,N′-diisopropylcarbodiimide (0.96 mL, 6.13 mmol). The resulting reaction mixture was stirred at room temperature for 24 h. After completion, the solvent was concentrated to obtain a residue that was then purified via preparatory HPLC (30-70% acetonitrile in water with 0.1% TFA). Fractions containing the desired product were combined and lyophilized to dryness to afford perfluorophenyl (S)-23-azido-18-(4-azidobutyl)-17,20-dioxo-4,7,10,13-tetraoxa-16,19-diazatricosanoate (Cpd. No. 26594) as a pale yellow viscous liquid. Yield: 0.160 g, 12%; ELSD m/z 697.4 [M+1]⁺; ¹H NMR (400 MHz, CDCl₃) δ 6.90 (bs, 1H), 6.35 (d, J=7.6 Hz, 1H), 4.46-4.41 (m, 1H), 3.90 (t, J=12.4 Hz, 1H), 3.67-3.61 (m, 12H), 3.56-3.45 (m, 5H), 3.64 (t, J=12.4 Hz, 2H), 3.28 (t, J=12.4 Hz, 1H), 2.97 (t, J=12.4 Hz, 2H), 2.42 (t, J=14.4 Hz, 2H), 1.95-1.75 (m, 4H), 1.61-1.37 (m, 4H).

### perfluorophenyl(18S,21S)-26-azido-18,21-bis(4-azidobutyl)-17,20,23-trioxo-4,7,10,13-tetraoxa-16,19,22-triazahexacosanoate (Cpd. No. 26604)

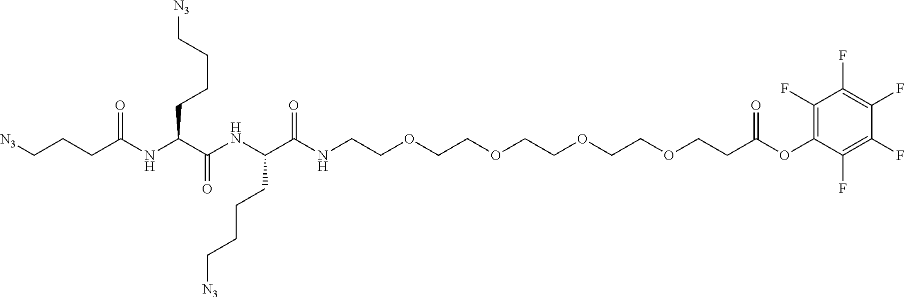 [SMILES: [N-]=[N+]=NCCCC[C@H](NC(=O)CCCN=[N+]=[N-])C(=O)N[C@@H](CCCCN=[N+]=[N-])C(=O)NCCOCCOCCOCCOCCC(=O)Oc1c(F)c(F)c(F)c(F)c1F]

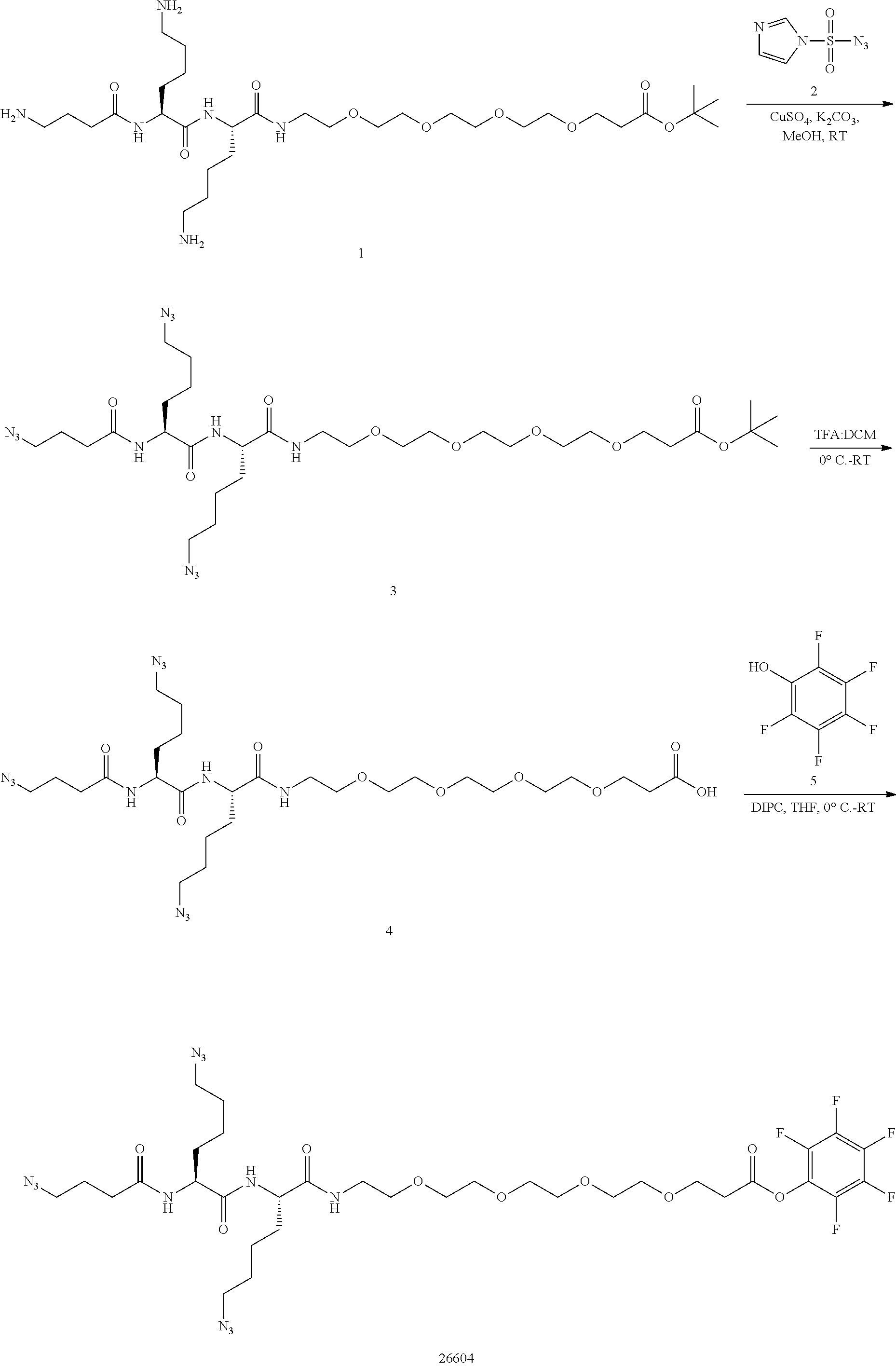 [SMILES: CC(C)(C)OC(=O)CCOCCOCCOCCOCCNC(=O)[C@H](CCCCN)NC(=O)[C@H](CCCCN)NC(=O)CCCN ; [N-]=[N+]=NS(=O)(=O)n1ccnc1 ; CC(C)(C)OC(=O)CCOCCOCCOCCOCCNC(=O)[C@H](CCCCN=[N+]=[N-])NC(=O)[C@H](CCCCN=[N+]=[N-])NC(=O)CCCN=[N+]=[N-] ; [N-]=[N+]=NCCCC[C@H](NC(=O)CCCN=[N+]=[N-])C(=O)N[C@@H](CCCCN=[N+]=[N-])C(=O)NCCOCCOCCOCCOCCC(=O)O ; Oc1c(F)c(F)c(F)c(F)c1F ; [N-]=[N+]=NCCCC[C@H](NC(=O)CCCN=[N+]=[N-])C(=O)N[C@@H](CCCCN=[N+]=[N-])C(=O)NCCOCCOCCOCCOCCC(=O)Oc1c(F)c(F)c(F)c(F)c1F]

### Synthesis of tert-butyl (18S,21S)-26-azido-18,21-bis(4-azidobutyl)-17,20,23-trioxo-4,7,10,13-tetraoxa-16,19,22-triazahexacosanoate (3)

To a stirred solution of tert-butyl (18S,21S)-26-amino-18,21-bis(4-aminobutyl)-17,20,23-trioxo-4,7,10,13-tetraoxa-16,19,22-triazahexacosanoate (1, 4.0 g, 6.03 mmol) in methanol (100.0 mL) was added potassium carbonate (7.51 g, 54.3 mmol), copper sulfate (0.452 g, 1.81 mmol), and 1H-imidazole-1-sulfonyl azide hydrochloride (2, 4.55 g, 21.7 mmol). The resulting reaction mixture was stirred at room temperature for 24 h. After completion, the reaction mixture was diluted with 1N hydrochloric acid solution up to pH 3 and extracted with ethyl acetate. The organic layer was dried over sodium sulfate, filtered, and concentrated under high vacuum to obtain a crude residue. The crude residue was purified via flash column chromatography (0-5% methanol in dichloromethane) to afford tert-butyl (18S,21S)-26-azido-18,21-bis(4-azidobutyl)-17,20,23-trioxo-4,7,10,13-tetraoxa-16,19,22-triazahexacosanoate (3) as a pale yellow viscous liquid. Yield: 2.50 g, 56%; ELSD m/z 741.4 [M+1]⁺.

### Synthesis of (18S,21S)-26-azido-18,21-bis(4-azidobutyl)-17,20,23-trioxo-4,7,10,13-tetraoxa-16,19,22-triazahexacosanoic acid (4)

To a stirred solution of tert-butyl (18S,21S)-26-azido-18,21-bis(4-azidobutyl)-17,20,23-trioxo-4,7,10,13-tetraoxa-16,19,22-triazahexacosanoate (3, 2.50 g, 3.37 mmol) in dichloromethane (30.0 mL) was added trifluoroacetic acid (5.0 mL) at 0° C. The resulting reaction mixture was stirred at room temperature under nitrogen for 4 h. After completion, the reaction mixture was concentrated and dried to afford (18S,21S)-26-azido-18,21-bis(4-azidobutyl)-17,20,23-trioxo-4,7,10,13-tetraoxa-16,19,22-triazahexacosanoic acid (4) as a pale yellow viscous liquid. Yield: 2.0 g, 86%; ELSD m/z 685.3 [M+1]⁺.

### Synthesis of perfluorophenyl (18S,21S)-26-azido-18,21-bis(4-azidobutyl)-17,20,23-trioxo-4,7,10,13-tetraoxa-16,19,22-triazahexacosanoate (Cpd. No. 26604)

To a stirred solution of (18S,21S)-26-azido-18,21-bis(4-azidobutyl)-17,20,23-trioxo-4,7,10,13-tetraoxa-16,19,22-triazahexacosanoic acid (4, 1.0 g, 1.46 mmol) in tetrahydrofuran (20.0 mL) at 0° C. was added 2,3,4,5,6-pentafluorophenol (5, 0.538 g, 2.92 mmol) and N, N′-diisopropylcarbodiimide (0.576 mL, 3.65 mmol). The resulting reaction mixture was stirred at room temperature for 24 h. After completion, the solvent was concentrated to obtain a residue that was then purified via preparatory HPLC (20-55% acetonitrile in water with 0.1% TFA). Fractions containing the desired product were combined and lyophilized to dryness to afford perfluorophenyl (18S,21S)-26-azido-18,21-bis(4-azidobutyl)-17,20,23-trioxo-4,7,10,13-tetraoxa-16,19,22-triazahexacosanoate (Cpd. No. 26604) as a pale yellow viscous liquid. Yield: 0.270 g, 21.7%; LCMS m/z 851.56 [M+1]⁺; ¹H NMR (400 MHz, CDCl₃) δ 6.71-6.66 (m, 2H), 6.33-6.13 (m, 1H), 4.44-4.36 (m, 2H), 3.89 (t, J=12.4 Hz, 1H), 3.67-3.61 (m, 12H), 3.56-3.54 (m, 2H), 3.47-3.44 (m, 2H), 3.37 (t, J=6.4 Hz, 2H), 3.28-3.24 (m, 4H), 2.96 (t, J=12.4 Hz, 2H), 2.34 (t, J=14.4 Hz, 2H), 1.96-1.80 (m, 4H), 1.67-1.59 (m, 6H), 1.43-1.36 (m, 4H).

### (2R,4S,5R,6R)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)-6-((1R,2R)-1,2,3-trihydroxypropyl)tetrahydro-2H-pyran-2-carboxylic acid (Cpd. No. 26614)

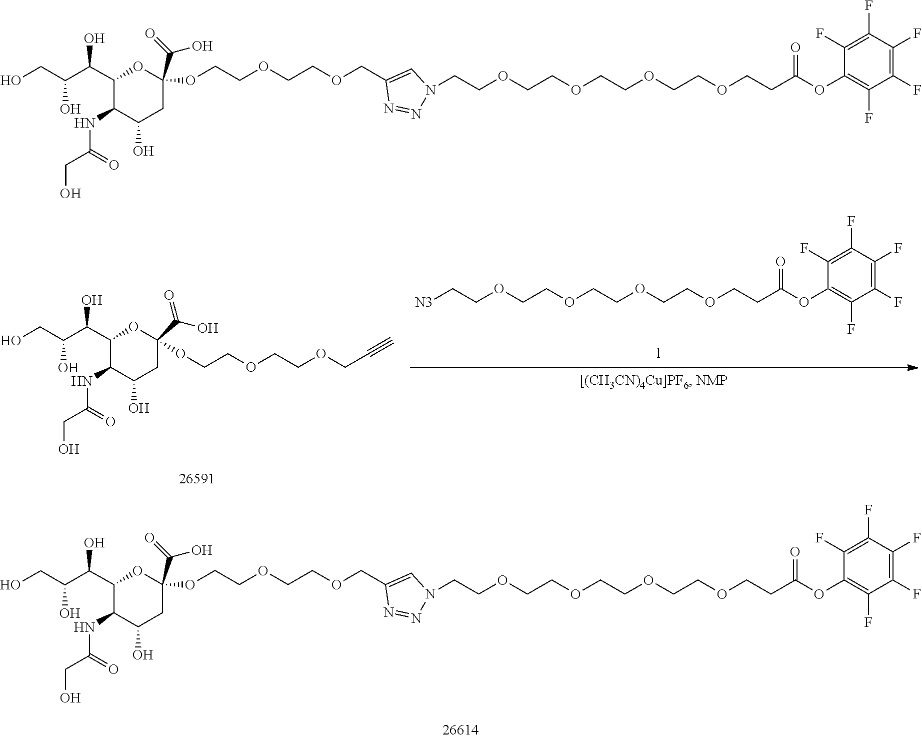 [SMILES: O=C(CO)N[C@H]1[C@H]([C@H](O)[C@H](O)CO)O[C@@](OCCOCCOCc2cn(CCOCCOCCOCCOCCC(=O)Oc3c(F)c(F)c(F)c(F)c3F)nn2)(C(=O)O)C[C@@H]1O ; C#CCOCCOCCO[C@]1(C(=O)O)C[C@H](O)[C@@H](NC(=O)CO)[C@H]([C@H](O)[C@H](O)CO)O1 ; [N-]=[N+]=NCCOCCOCCOCCOCCC(=O)Oc1c(F)c(F)c(F)c(F)c1F ; O=C(CO)N[C@H]1[C@H]([C@H](O)[C@H](O)CO)O[C@@](OCCOCCOCc2cn(CCOCCOCCOCCOCCC(=O)Oc3c(F)c(F)c(F)c(F)c3F)nn2)(C(=O)O)C[C@@H]1O]

### Synthesis of (2R,4S,5R,6R)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)-6-((1R,2R)-1,2,3-trihydroxypropyl)tetrahydro-2H-pyran-2-carboxylic acid (Cpd. No. 26614)

To a solution of (2R,4S,5R,6R)-4-hydroxy-5-(2-hydroxyacetamido)-2-(2-(2-(prop-2-yn-1-yloxy)ethoxy)ethoxy)-6-((1R,2R)-1,2,3-trihydroxypropyl)tetrahydro-2H-pyran-2-carboxylic acid (26591, 1.00 eq, 24.5 mg, 0.0542 mmol) in NMP (0.3 mL) in a 1 dram vial with a stirbar was added a solution of perfluorophenyl 1-azido-3,6,9,12-tetraoxapentadecan-15-oate (1, 1.10 eq, 27.3 mg, 0.0596 mmol) in NMP (0.3 mL) followed by tetrakis(acetonitrile)copper(I) hexafluorophosphate (2.50 eq, 50.5 mg, 0.136 mmol). The resulting clear green solution was capped and stirred at room temperature for 10 min. The reaction was diluted with acetic acid, filtered, and purified via preparatory HPLC (15-65% acetonitrile in water with 0.1% TFA). Fractions containing the desired product were combined and lyophilized to dryness to afford (2R,4S,5R,6R)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)-6-((1R,2R)-1,2,3-trihydroxypropyl)tetrahydro-2H-pyran-2-carboxylic acid (Cpd. No. 26614) as a sticky white solid. Yield: 28.1 mg, 57%; LCMS m/z 909.6 [M+1]⁺; ¹H NMR (300 MHz, DMSO-d₆ with D₂O) δ 7.99 (s, 1H), 7.80 (bs, 1H), 5.61 (bs, 1H), 4.52-4.40 (m, 4H), 3.87-2.69 (m, 35H), 1.56-1.43 (m, 2H).

### (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(4-hydroxy-5-(2-hydroxyacetamido)-6-((1R,2R)-1,2,3-trihydroxypropyl)tetrahydro-2H-pyran-2-carboxylic acid) (Cpd. No. 26615)

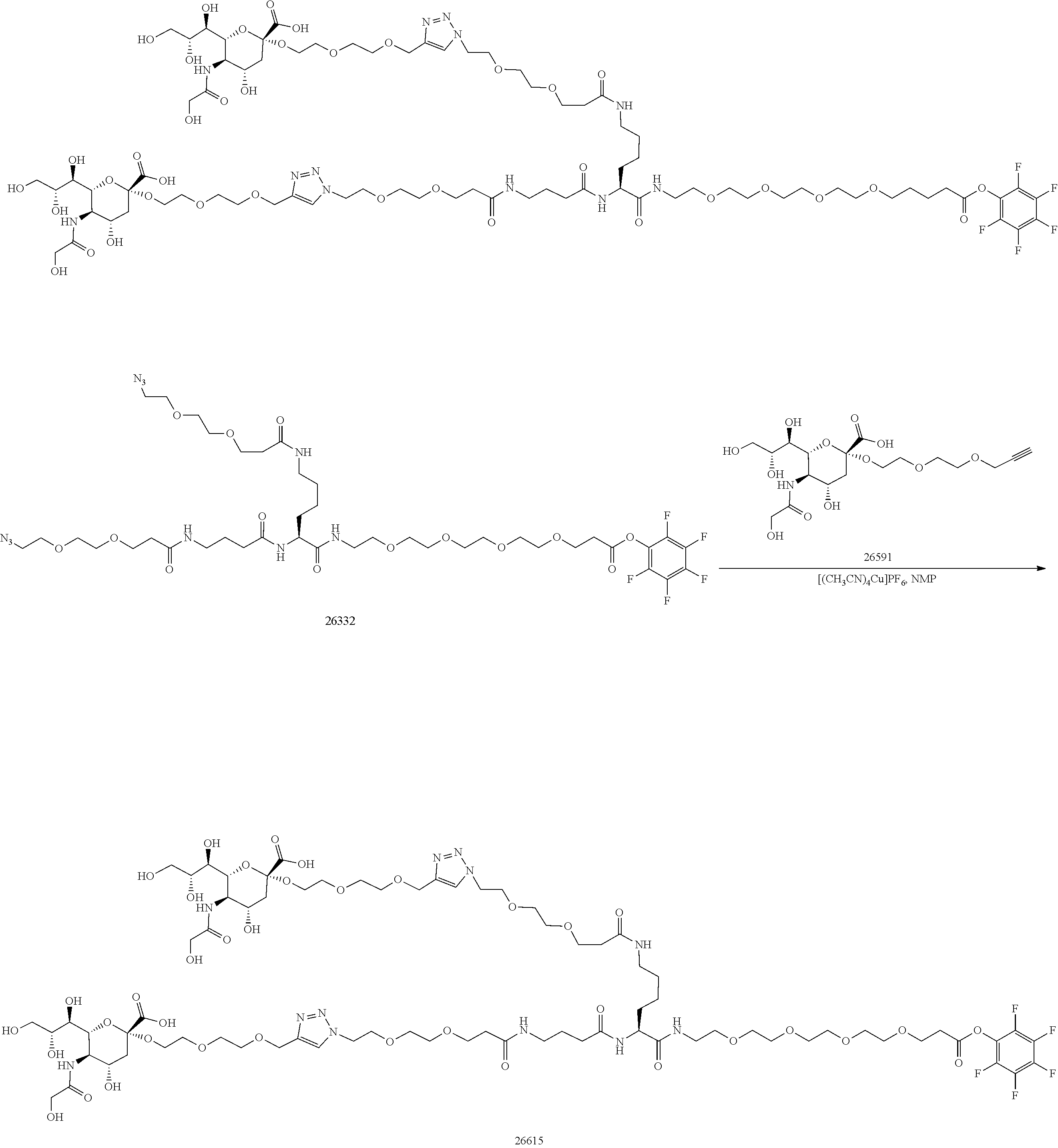

### Synthesis of (2R,2′R,4S,4'S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(4-hydroxy-5-(2-hydroxyacetamido)-6-((1R,2R)-1,2,3-trihydroxypropyl)tetrahydro-2H-pyran-2-carboxylic acid) (Cpd. No. 26615)

To a solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq, 32.1 mg, 0.0317 mmol) and (2R,4S,5R,6R)-4-hydroxy-5-(2-hydroxyacetamido)-2-(2-(2-(prop-2-yn-1-yloxy)ethoxy)ethoxy)-6-((1R,2R)-1,2,3-trihydroxypropyl)tetrahydro-2H-pyran-2-carboxylic acid (26591, 2.10 eq, 30.0 mg, 0.0665 mmol) in NMP (0.5 mL) in a 1 dram vial with a stirbar was added tetrakis(acetonitrile)copper(I) hexafluorophosphate (5.00 eq, 59.0 mg, 0.158 mmol). The resulting colourless solution was capped and stirred at room temperature for 10 min (slowly turned green). The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(4-hydroxy-5-(2-hydroxyacetamido)-6-((1R,2R)-1,2,3-trihydroxypropyl)tetrahydro-2H-pyran-2-carboxylic acid) (Cpd. No. 26615) as a white solid. Yield: 32.9 mg, 54%; LCMS m/z 1917.5 [M+1]+; ¹H NMR (300 MHz, DMSO-d₆ with D₂O) δ 7.98 (s, 2H), 7.82 (bs, 2H), 4.52-4.41 (m, 8H), 4.12-2.69 (m, 79H), 2.31-2.19 (m, 4H), 2.14-2.04 (m, 2H), 1.65-1.38 (m, 4H), 1.38-1.09 (m, 4H).

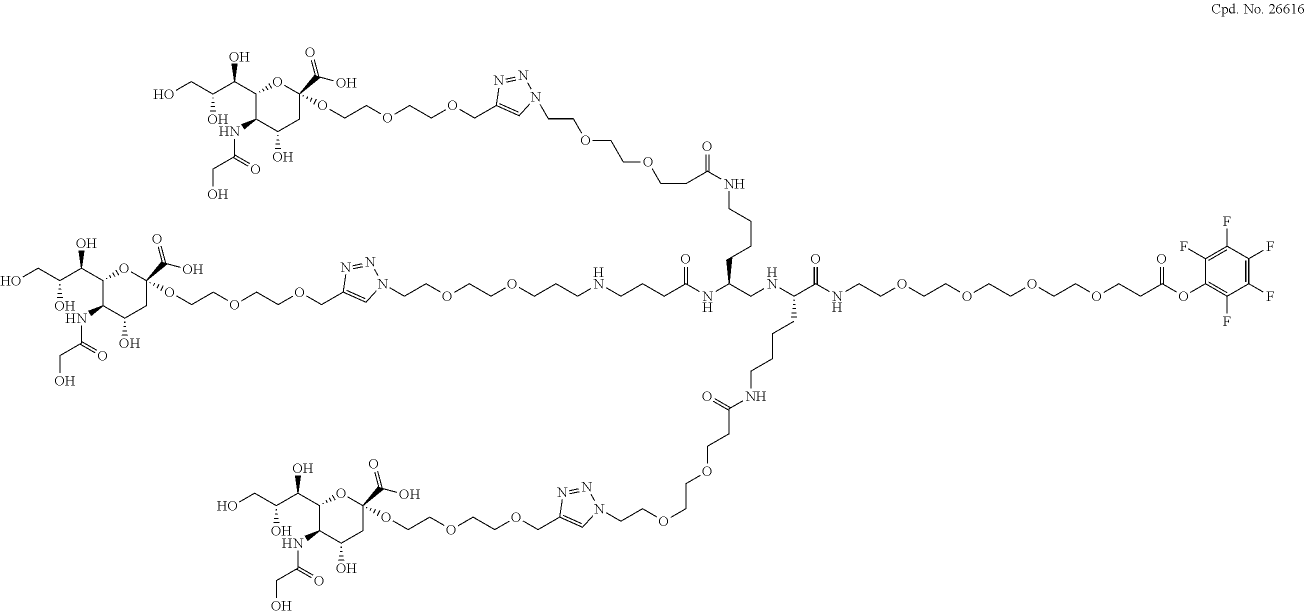 [SMILES: O=C(CCOCCOCCn1cc(COCCOCCO[C@]2(C(=O)O)C[C@H](O)[C@@H](NC(=O)CO)[C@H]([C@H](O)[C@H](O)CO)O2)nn1)NCCCC[C@@H](CN[C@@H](CCCCNC(=O)CCOCCOCCn1cc(COCCOCCO[C@]2(C(=O)O)C[C@H](O)[C@@H](NC(=O)CO)[C@H]([C@H](O)[C@H](O)CO)O2)nn1)C(=O)NCCOCCOCCOCCOCCC(=O)Oc1c(F)c(F)c(F)c(F)c1F)NC(=O)CCCNCCCOCCOCCn1cc(COCCOCCO[C@]2(C(=O)O)C[C@H](O)[C@@H](NC(=O)CO)[C@H]([C@H](O)[C@H](O)CO)O2)nn1]

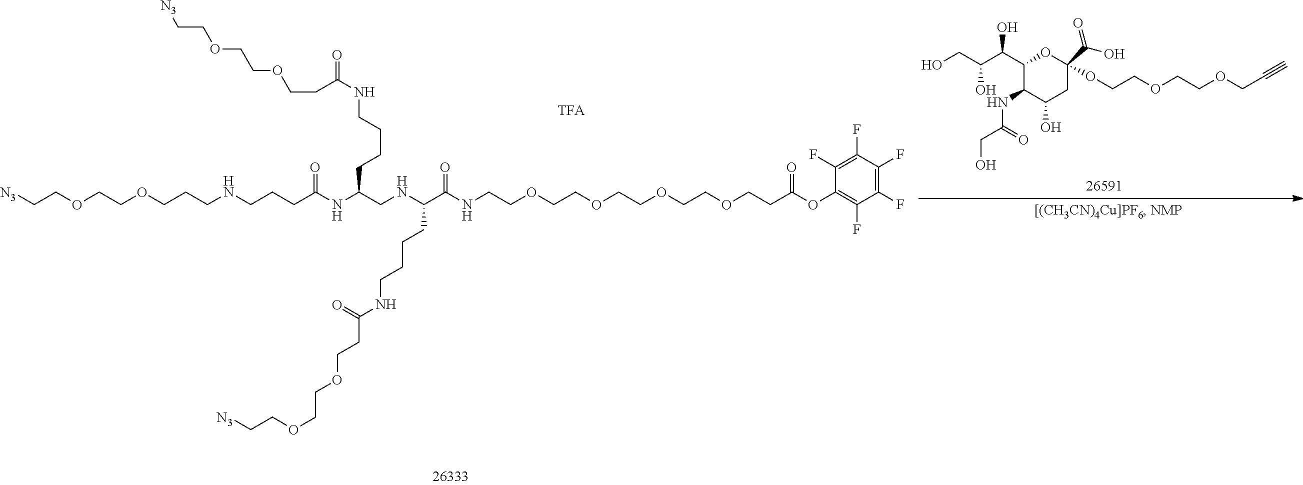 [SMILES: [N-]=[N+]=NCCOCCOCCCNCCCC(=O)N[C@@H](CCCCNC(=O)CCOCCOCCN=[N+]=[N-])CN[C@@H](CCCCNC(=O)CCOCCOCCN=[N+]=[N-])C(=O)NCCOCCOCCOCCOCCC(=O)Oc1c(F)c(F)c(F)c(F)c1F ; C#CCOCCOCCO[C@]1(C(=O)O)C[C@H](O)[C@@H](NC(=O)CO)[C@H]([C@H](O)[C@H](O)CO)O1]

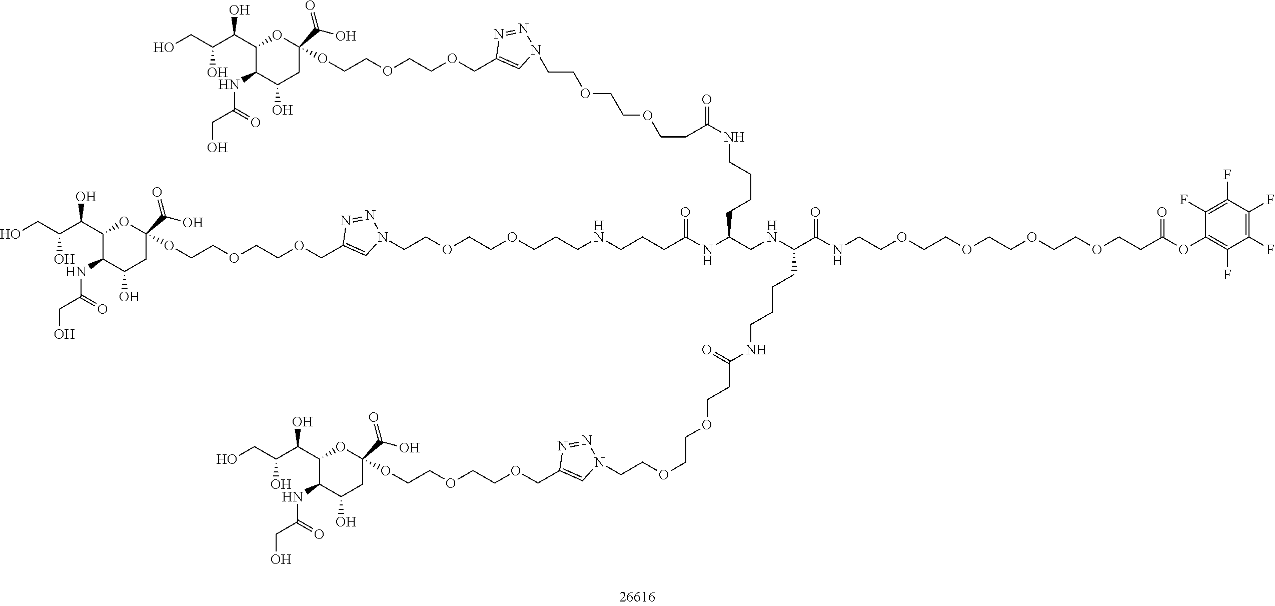 [SMILES: O=C(CCOCCOCCn1cc(COCCOCCO[C@]2(C(=O)O)C[C@H](O)[C@@H](NC(=O)CO)[C@H]([C@H](O)[C@H](O)CO)O2)nn1)NCCCC[C@@H](CN[C@@H](CCCCNC(=O)CCOCCOCCn1cc(COCCOCCO[C@]2(C(=O)O)C[C@H](O)[C@@H](NC(=O)CO)[C@H]([C@H](O)[C@H](O)CO)O2)nn1)C(=O)NCCOCCOCCOCCOCCC(=O)Oc1c(F)c(F)c(F)c(F)c1F)NC(=O)CCCNCCCOCCOCCn1cc(COCCOCCO[C@]2(C(=O)O)C[C@H](O)[C@@H](NC(=O)CO)[C@H]([C@H](O)[C@H](O)CO)O2)nn1]

Synthesis of Cpd. No. 26616

To a solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq, 32.3 mg, 0.0224 mmol) and ((2R,4S,5R,6R)-4-hydroxy-5-(2-hydroxyacetamido)-2-(2-(2-(prop-2-yn-1-yloxy)ethoxy)ethoxy)-6-((1R,2R)-1,2,3-trihydroxypropyl)tetrahydro-2H-pyran-2-carboxylic acid (26591, 3.10 eq, 31.3 mg, 0.0694 mmol) in NMP (0.5 mL) in a 1 dram vial with a stirbar was added tetrakis(acetonitrile)copper(I) hexafluorophosphate (7.50 eq, 62.6 mg, 0.168 mmol). The resulting clear green solution was capped and stirred at room temperature for 10 min. The reaction was diluted with a mixture of 70% acetic acid in NMP, filtered, and purified via preparatory HPLC (15-65% acetonitrile in water with 0.1% TFA). Fractions containing the desired product were combined and lyophilized to dryness to afford Cpd. No. 26616 as a white solid. Yield: 31.1 mg, 52%; LCMS m/z 1341.6 [M/2+1]+; ¹H NMR (300 MHz, DMSO-d₆ with D₂O) δ 7.99 (s, 3H), 7.82 (bs, 3H), 4.53-4.41 (m, 12H), 4.15-2.70 (m, 107H), 2.31-2.19 (m, 6H), 2.14-2.04 (m, 2H), 1.66-1.38 (m, 8H), 1.38-1.08 (m, 8H).

### Perfluorophenyl N2-(N2-(4-azidobutanoyl)-N6-diazo-L-lysyl)-N6-diazo-L-lysinate (Cpd. No. 26634) and N2-(N2-(4-azidobutanoyl)-N6-diazo-L-lysyl)-N6-diazo-L-lysine (Cpd. No. 26728)

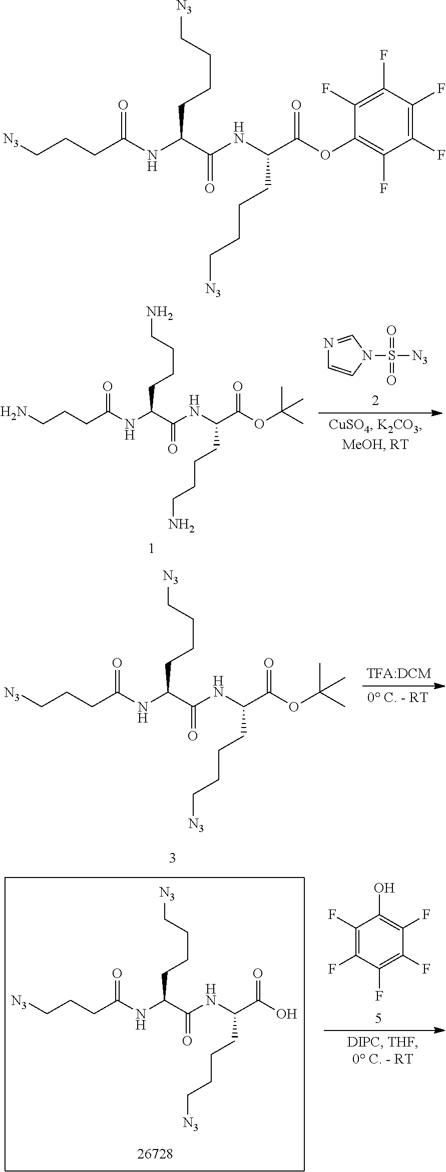 [SMILES: [N-]=[N+]=NCCCC[C@H](NC(=O)CCCN=[N+]=[N-])C(=O)N[C@@H](CCCCN=[N+]=[N-])C(=O)Oc1c(F)c(F)c(F)c(F)c1F ; CC(C)(C)OC(=O)[C@H](CCCCN)NC(=O)[C@H](CCCCN)NC(=O)CCCN ; [N-]=[N+]=NS(=O)(=O)n1ccnc1 ; CC(C)(C)OC(=O)[C@H](CCCCN=[N+]=[N-])NC(=O)[C@H](CCCCN=[N+]=[N-])NC(=O)CCCN=[N+]=[N-] ; [N-]=[N+]=NCCCC[C@H](NC(=O)[C@H](CCCCN=[N+]=[N-])NC(=O)CCCN=[N+]=[N-])C(=O)O ; Oc1c(F)c(F)c(F)c(F)c1F]

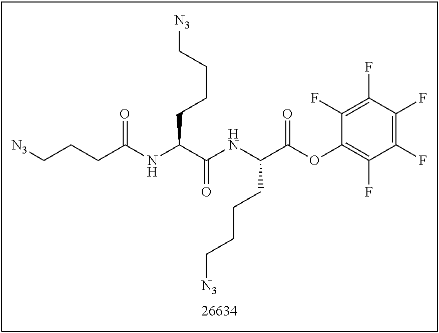 [SMILES: [N-]=[N+]=NCCCC[C@H](NC(=O)CCCN=[N+]=[N-])C(=O)N[C@@H](CCCCN=[N+]=[N-])C(=O)Oc1c(F)c(F)c(F)c(F)c1F]

### Synthesis of tert-butyl N2-(N2-(4-azidobutanoyl)-N6-diazo-L-lysyl)-N6-diazo-L-lysinate (3)

To a stirred solution of tert-butyl (4-aminobutanoyl)-L-lysyl-L-lysinate (1, 5.0 g, 12.0 mmol) in methanol (100.0 mL) was added potassium carbonate (15.0 g, 108 mmol), copper sulfate (0.901 g, 3.61 mmol), and 1H-imidazole-1-sulfonyl azide hydrochloride (2, 7.29 g, 42.1 mmol). The resulting reaction mixture was stirred at room temperature for 24 h. After completion, the reaction mixture was diluted with 1N hydrochloric acid solution up to pH 3 and extracted with ethyl acetate. The organic layer was dried over sodium sulfate, filtered, and concentrated under high vacuum to obtain a crude residue. The crude residue was purified via flash column chromatography (30-50% ethyl acetate in hexanes) to afford tert-butyl N2-(N2-(4-azidobutanoyl)-N6-diazo-L-lysyl)-N6-diazo-L-lysinate (3) as a pale yellow viscous liquid. Yield: 1.80 g, 30%; ELSD m/z 494.2 [M+1]⁺.

### Synthesis of N2-(N2-(4-azidobutanoyl)-N6-diazo-L-lysyl)-N6-diazo-L-lysine (Cpd. No. 26728)

To a stirred solution of tert-butyl N2-(N2-(4-azidobutanoyl)-N6-diazo-L-lysyl)-N6-diazo-L-lysinate (3, 1.0 g, 2.03 mmol) in dichloromethane (10 mL) at 0° C. was added trifluoroacetic acid (3.0 mL). The resulting reaction mixture was stirred at room temperature under nitrogen for 5 h. After completion, the reaction mixture was concentrated and dried to afford N2-(N2-(4-azidobutanoyl)-N6-diazo-L-lysyl)-N6-diazo-L-lysine (Cpd. No. 26728) as a pale yellow viscous liquid. Yield: 0.70 g, 79%; ELSD m/z 438.2 [M+1]⁺, ¹H NMR (400 MHz, DMSO-d₆) δ 12.58 (bs, 1H), 8.15 (d, J=7.6 Hz, 1H), 8.00 (d, J=8.4 Hz, 1H), 4.37-4.15 (m, 2H), 3.32-3.29 (m, 6H), 2.23-2.18 (m, 2H), 1.75-1.23 (m, 16H).

### Synthesis of Perfluorophenyl N2-(N2-(4-azidobutanoyl)-N6-diazo-L-lysyl)-N6-diazo-L-lysinate (Cpd. No. 26634)

To a stirred solution of N2-(N2-(4-azidobutanoyl)-N6-diazo-L-lysyl)-N6-diazo-L-lysine (26728, 0.50 g, 1.14 mmol) in tetrahydrofuran (5.0 mL) at 0° C. was added 2,3,4,5,6-pentafluorophenol (5, 0.421 g, 2.29 mmol) and N, N′-diisopropylcarbodiimide (0.440 mL, 2.86 mmol). The resulting reaction mixture was stirred at room temperature for 16 h. After completion, the solvent was concentrated to obtain a residue which was then purified via preparatory HPLC (30-65% acetonitrile in water with 0.1% TFA). Fractions containing the desired product were combined and lyophilized to dryness to afford Perfluorophenyl N2-(N2-(4-azidobutanoyl)-N6-diazo-L-lysyl)-N6-diazo-L-lysinate (Cpd. No. 26634) as an off white sticky solid. Yield: 0.080 g, 11.5%; ELSD m/z 604.4 [M+1]^{+ 1}H NMR (400 MHz, DMSO-d₆) δ 8.43-8.38 (m, 1H), 8.12-7.97 (m 1H), 4.62-4.59 (m, 1H), 4.48-4.47 (m, 1H), 2.24-2.19 (m, 3H), 1.78-1.23 (m, 19H).

### (2S,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid (Cpd. No. 26635)

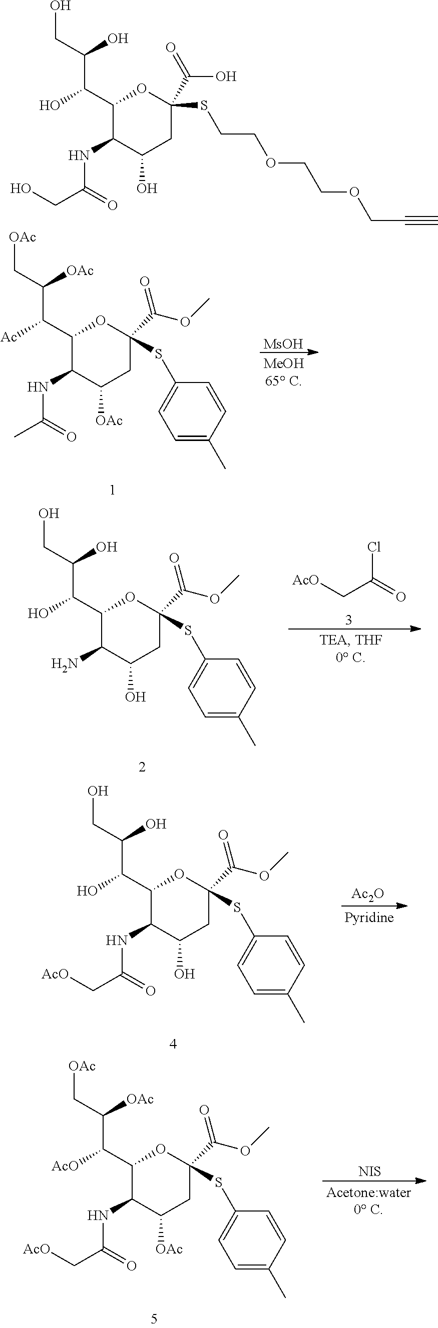

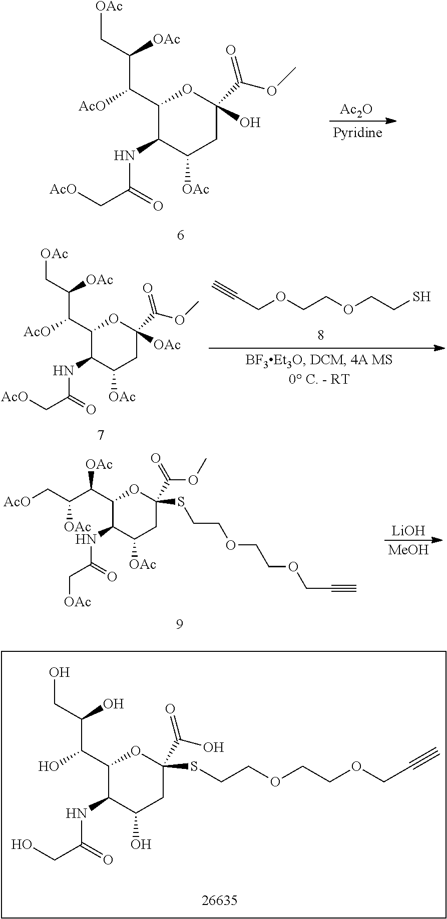

### Synthesis of methyl (2R,4S,5R,6R)-5-amino-4-hydroxy-2-(p-tolylthio)-6-((1R,2R)-1,2,3-trihydroxypropyl)tetrahydro-2H-pyran-2-carboxylate (2)

To a stirred solution of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (1, 10.0 g, 16.73 mmol) in methanol (100.0 mL) was added methane sulfonic acid (6.52 mL, 100.4 mmol) dropwise at 0° C. The resulting reaction mixture was stirred at 63° C. for 30 h and progress of the reaction was monitored by LC-MS. After completion, the reaction mixture was cooled to 0° C. and quenched with triethylamine (^{˜}15.0 mL, pH 7). The mixture was concentrated under reduced pressure to obtain crude methyl (2R,4S,5R,6R)-5-amino-4-hydroxy-2-(p-tolylthio)-6-((1R,2R)-1,2,3-trihydroxypropyl)tetrahydro-2H-pyran-2-carboxylate (2) as a light brown gel. Yield: 6.0 g, 92.6%; LC-MS (ESI) m/z 388.14 [M+1]⁺.

### Synthesis of methyl (2R,4S,5R,6R)-5-(2-acetoxyacetamido)-4-hydroxy-2-(p-tolylthio)-6-((1R,2R)-1,2,3-trihydroxypropyl)tetrahydro-2H-pyran-2-carboxylate (4)

In an inert atmosphere, crude methyl (2R,4S,5R,6R)-5-amino-4-hydroxy-2-(p-tolylthio)-6-((1R,2R)-1,2,3-trihydroxypropyl)tetrahydro-2H-pyran-2-carboxylate (2, 6.0 g, 15.49 mmol) was dissolved under stirring in dry tetrahydrofuran (60.0 mL) and cooled at 0° C. To this solution, triethylamine (6.34 mL, 46.46 mmol) followed by 2-chloro-2-oxoethyl acetate (3, 1.66 mL, 15.49 mmol) was added slowly at 0° C.; The reaction was stirred at 0° C. and progress was monitored by TLC. After completion, the mixture was concentrated under reduced pressure to obtain a crude residue which was then purified via column chromatography (60-75% ethyl acetate in hexanes) to afford methyl (2R,4S,5R,6R)-5-(2-acetoxyacetamido)-4-hydroxy-2-(p-tolylthio)-6-((1R,2R)-1,2,3-trihydroxypropyl)tetrahydro-2H-pyran-2-carboxylate (4) as a white solid. Yield: 4.80 g, 63.58%; LCMS (ESI) m/z 488.52 [M+1]⁺.

### Synthesis of (1S,2R)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2,3-triyl triacetate (5)

To a stirred solution of methyl (2R,4S,5R,6R)-5-(2-acetoxyacetamido)-4-hydroxy-2-(p-tolylthio)-6-((1R,2R)-1,2,3-trihydroxypropyl)tetrahydro-2H-pyran-2-carboxylate (4, 4.80 g, 9.85 mmol) in pyridine (50.0 mL) was dropwise added acetic anhydride (9.31 mL, 98.46 mmol) at 0° C. over 30 min. The reaction mixture was stirred overnight and allowed to warm to room temperature. Progress of the reaction was monitored by TLC and LC-MS. After completion, volatiles were removed under vacuum to obtain a crude thick syrup. The thick syrup was poured into a separatory funnel with ethyl acetate (240.0 mL) and washed with 1N HCl solution, followed by saturated sodium sulfate solution and DM water. The organic layer was dried over anhydrous sodium sulfate and concentrated under reduced pressure to obtain a crude thick syrup. The syrup was purified via column chromatography (60-70% ethyl acetate in hexanes) to afford (1S,2R)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2,3-triyl triacetate (5) as a white solid. Yield: 3.40 g, 52.67%; LCMS (ESI) m/z 654.2 [M−1]⁻.

### Synthesis of (1S,2R)-1-((2R,3R,4S,6S)-4-acetoxy-3-(2-acetoxyacetamido)-6-hydroxy-6-(methoxycarbonyl)tetrahydro-2H-pyran-2-yl)propane-1,2,3-triyl triacetate (6)

A stirred solution of (1S,2R)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2,3-triyl triacetate (5, 3.40 g, 5.19 mmol) in acetone:water (9:1, 35.0 mL) was cooled to 0° C. To this solution, N-iodosuccinimide (4.08 g, 18.15 mmol) was added and the reaction mixture was maintained at 0° C. for 3 h. Reaction progress was monitored by LC-MS/TLC and after completion, saturated aqueous solution of sodium metabisulfide (10.0 mL) and ethyl acetate (30.0 mL) were added. The reaction mixture was stirred for another 10 min and transferred to a separatory funnel. The organic layer was separated and the aqueous phase was extracted with ethyl acetate (20 mL). The organic layers were combined and washed sequentially with saturated sodium bicarbonate solution and DM water. The organic layer was dried over anhydrous sodium sulfate and concentrated on a rotary evaporator to obtain a thick syrup. The thick syrup was purified via column chromatography (55-65% ethyl acetate in hexanes) to afford (1S,2R)-1-((2R,3R,4S,6S)-4-acetoxy-3-(2-acetoxyacetamido)-6-hydroxy-6-(methoxycarbonyl)tetrahydro-2H-pyran-2-yl)propane-1,2,3-triyl triacetate (6) as a white solid. Yield: 1.90 g, 66.68%; LCMS (ESI) m/z 550.48 [M+1]+.

### Synthesis of (1S,2R)-1-((2R,3R,4S,6R)-4,6-diacetoxy-3-(2-acetoxyacetamido)-6-(methoxycarbonyl)tetrahydro-2H-pyran-2-yl)propane-1,2,3-triyl triacetate (7)

To a stirred solution of (1S,2R)-1-((2R,3R,4S,6S)-4-acetoxy-3-(2-acetoxyacetamido)-6-hydroxy-6-(methoxycarbonyl)tetrahydro-2H-pyran-2-yl)propane-1,2,3-triyl triacetate (6, 1.90 g, 3.46 mmol) in pyridine (20.0 mL) was dropwise added acetic anhydride (0.653 mL, 6.92 mmol) at 0° C. over 30 min. The reaction mixture was stirred overnight and allowed to warm to room temperature. The progress of the reaction was monitored by TLC and LC-MS. After completion, volatiles were removed under vacuum to obtain a crude thick syrup. The thick syrup was then poured into a separatory funnel with ethyl acetate (30.0 mL) and washed with 1N HCl solution followed by saturated sodium sulfate solution and DM water. The organic layer was dried over anhydrous sodium sulfate and concentrated under reduced pressure to obtain a crude thick syrup. The syrup was purified via column chromatography (45-55% ethyl acetate in hexanes) to afford (1S,2R)-1-((2R,3R,4S,6R)-4,6-diacetoxy-3-(2-acetoxyacetamido)-6-(methoxycarbonyl)tetrahydro-2H-pyran-2-yl)propane-1,2,3-triyl triacetate (7) as a white solid. Yield: 1.50 g, 73.34%; LCMS (ESI) m/z 591.52 [M+1]⁺.

### Synthesis of (1S,2R)-1-((2R,3R,4S,6R)-4-acetoxy-3-(2-acetoxyacetamido)-6-(methoxycarbonyl)-6-((2-(2-(prop-2-yn-1-yloxy)ethoxy)ethyl)thio)tetrahydro-2H-pyran-2-yl)propane-1,2,3-triyl triacetate (9)

In an inert atmosphere, (1S,2R)-1-((2R,3R,4S,6R)-4,6-diacetoxy-3-(2-acetoxyacetamido)-6-(methoxycarbonyl)tetrahydro-2H-pyran-2-yl)propane-1,2,3-triyl triacetate (7, 1.0 g, 1.69 mmol) and 2-(2-(prop-2-yn-1-yloxy)ethoxy)ethane-1-thiol (8, 1.35 g, 8.45 mmol) were dissolved in dry dichloromethane (10.0 mL) under stirring at room temperature. To this solution, activated powdered 4 Å molecular sieves (1.0 g 100% w/w) were added at room temperature and the reaction mixture was stirred for 30 min. The reaction mixture was cooled to 0° C. and BF₃·Et₂O (1.50 mL, 5.07 mmol) was added drop-wise at 0° C. over 15 min. The mixture was stirred at room temperature for 16 h. The reaction mixture was monitored by TLC/LC-MS and after completion the reaction mixture was quenched by triethylamine up to neutral pH. The reaction mixture was filtered and washed with aqueous saturated sodium bicarbonate solution. The organic layer was separated, dried over anhydrous sodium sulfate, and concentrated on a rotary evaporator to obtain a crude thick syrup. The thick syrup was purified via column chromatography (60-70% ethyl acetate in hexanes) to afford (1S,2R)-1-((2R,3R,4S,6R)-4-acetoxy-3-(2-acetoxyacetamido)-6-(methoxycarbonyl)-6-((2-(2-(prop-2-yn-1-yloxy)ethoxy)ethyl)thio)tetrahydro-2H-pyran-2-yl)propane-1,2,3-triyl triacetate (9) as a white solid. Yield: 0.70 g, 59.86%; LCMS (ESI) m/z 692.70 [M+1]⁺.

### Synthesis of (2R,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid (Cpd. No. 26635)

To a stirred solution of (1S,2R)-1-((2R,3R,4S,6R)-4-acetoxy-3-(2-acetoxyacetamido)-6-(methoxycarbonyl)-6-((2-(2-(prop-2-yn-1-yloxy)ethoxy)ethyl)thio)tetrahydro-2H-pyran-2-yl)propane-1,2,3-triyl triacetate (9, 0.70 g, 1.01 mmol) in methanol (10.0 mL) at 0° C. was added lithium hydroxide (0.072 g, 3.04 mmol). The reaction mixture was stirred for 6 h and allowed to warm to room temperature. Progress of the reaction was monitored by TLC and LC-MS. After completion, Dowex-Hydrogen form was added up to neutral pH and the reaction mixture was filtered through a sintered funnel. The filtrate was removed under vacuum to obtain a crude thick syrup that was purified via preparatory HPLC (20-45% acetonitrile in water with 0.1% TFA). Fractions containing the desired product were combined and lyophilized to dryness to afford (2R,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid (Cpd. No. 26635) as an amorphous solid. Yield: 0.078 g, 16.49%; LCMS (ESI) m/z 468.28 [M+1]⁺. ¹H NMR (400 MHz, methanol-d₄) δ 4.26 (d, J=10.4 Hz, 1H), 4.18 (d, J=2.4 Hz, 2H), 4.14 (dd, J=10.4 & 4.8 Hz, 2H), 4.04 (s, 2H), 3.90 (t, J=10.4 Hz, 2H), 3.82-3.78 (m, 2H), 3.68-3.52 (m, 8H), 2.87-2.83 (m, 3H), 2.48 (dd, J=14.0, 4.8 Hz, 1H), 1.95 (t, J=14.0 Hz, 1H).

### N2-(4-azidobutanoyl)-N6-diazo-L-lysine (Cpd. No. 26727)

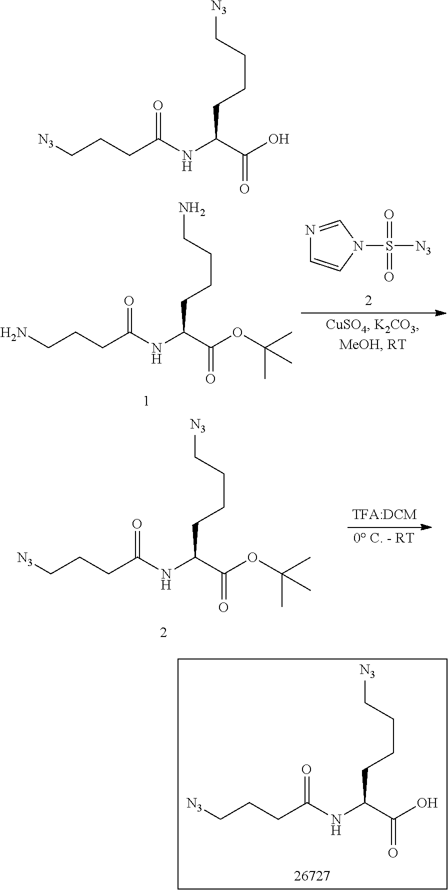 [SMILES: [N-]=[N+]=NCCCC[C@H](NC(=O)CCCN=[N+]=[N-])C(=O)O ; CC(C)(C)OC(=O)[C@H](CCCCN)NC(=O)CCCN ; [N-]=[N+]=NS(=O)(=O)n1ccnc1 ; CC(C)(C)OC(=O)[C@H](CCCCN=[N+]=[N-])NC(=O)CCCN=[N+]=[N-] ; [N-]=[N+]=NCCCC[C@H](NC(=O)CCCN=[N+]=[N-])C(=O)O]

### Synthesis of tert-butyl N2-(4-azidobutanoyl)-N6-diazo-L-lysinate (3)

To a stirred solution of tert-butyl (4-aminobutanoyl)-L-lysinate (1, 2.4 g, 8.35 mmol) in methanol (40.0 mL) was added potassium carbonate (6.92 g, 50.1 mmol), copper sulfate (0.417 g, 1.67 mmol), and 1H-imidazole-1-sulfonyl azide hydrochloride (2, 3.61 g, 20.9 mmol). The resulting reaction mixture was stirred at room temperature for 24 h. After completion, the reaction mixture was diluted with 1N hydrochloric acid solution up to pH 3 and extracted with ethyl acetate. The organic layer was dried over sodium sulfate, filtered, and concentrated under high vacuum to obtain a crude residue. The crude residue was purified via flash column chromatography (30-50% ethyl acetate in hexanes) to afford tert-butyl N2-(4-azidobutanoyl)-N6-diazo-L-lysinate (3) as a pale yellow viscous liquid. Yield: 1.0 g, 35%; ELSD m/z 340.5 [M+1]⁺.

### Synthesis of N2-(4-azidobutanoyl)-N6-diazo-L-lysine (Cpd. No. 26727)

To a stirred solution of tert-butyl N2-(4-azidobutanoyl)-N6-diazo-L-lysinate (3, 1.0 g, 2.95 mmol) in dichloromethane (10.0 mL) was added trifluoroacetic acid (2.0 mL) at 0° C. The resulting mixture was stirred at room temperature under nitrogen for 4 h. After completion, the reaction mixture was concentrated and dried to afford N2-(4-azidobutanoyl)-N6-diazo-L-lysine (Cpd. No. 26727) as a pale yellow viscous liquid. Yield: 0.80 g, 95%; ELSD m/z 284.0 [M+1]⁺. ¹H NMR (400 MHz, DMSO-d₆) δ 12.08 (bs, 1H), 8.14 (d, J=7.6 Hz, 1H), 4.19-4.13 (m, 1H), 3.33-3.29 (m, 4H), 2.22 (t, J=14.4 Hz, 2H), 1.78-1.33 (m, 8H).

### (2S,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid (Cpd. No. 26729)

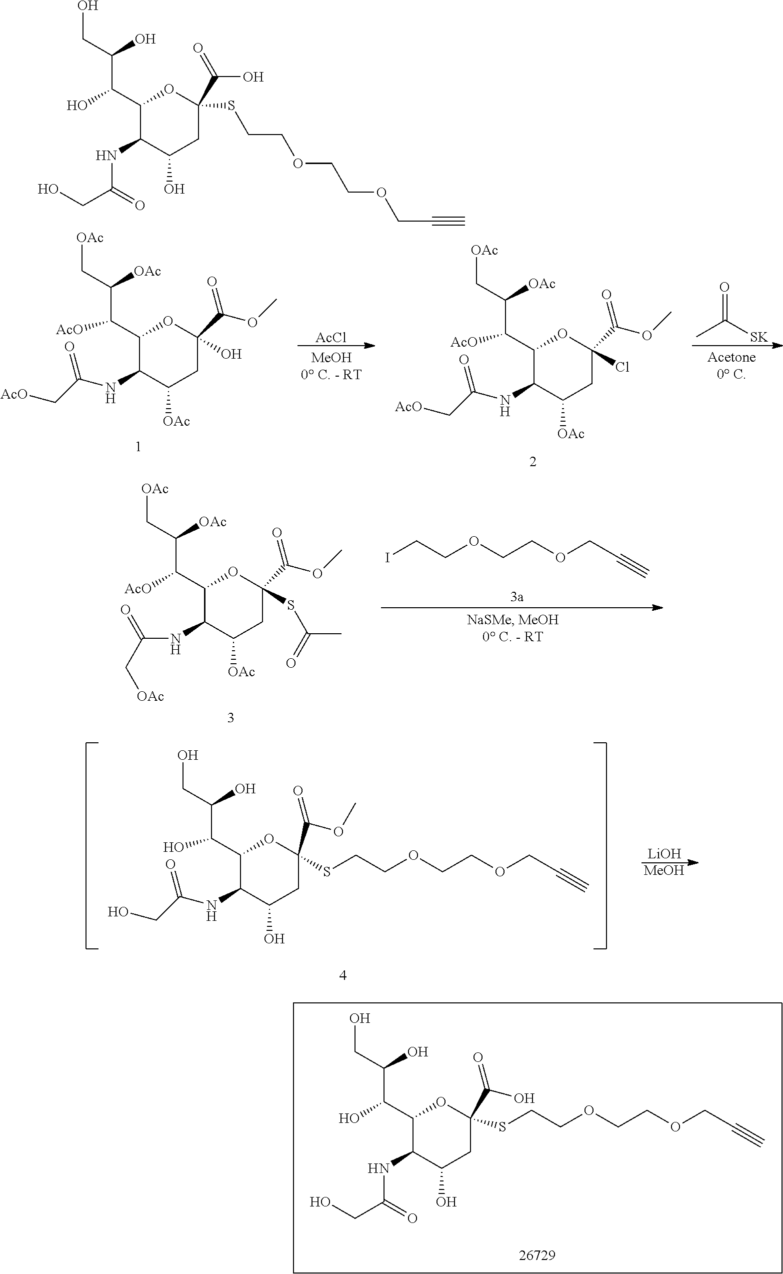

### Synthesis of methyl (2R,4S,5R,6R)-5-amino-4-hydroxy-2-(p-tolylthio)-6-((1R,2R)-1,2,3-trihydroxypropyl)tetrahydro-2H-pyran-2-carboxylate (2)

To a stirred solution of (1S,2R)-1-((2R,3R,4S,6R)-4-acetoxy-3-(2-acetoxyacetamido)-6-hydroxy-6-(methoxycarbonyl)tetrahydro-2H-pyran-2-yl)propane-1,2,3-triyl triacetate (1, 1.0 g, 1.82 mmol) in methanol (150.0 mL) was added anhydrous methanol (0.6 mL) dropwise at 0° C. The resulting reaction mixture was stirred at room temperature for 24 h and progress of the reaction was monitored by TLC. After completion, the reaction mixture was concentrated under reduced pressure to obtain crude (1S,2R)-1-((2R,3R,4S,6R)-4-acetoxy-3-(2-acetoxyacetamido)-6-chloro-6-(methoxycarbonyl)tetrahydro-2H-pyran-2-yl)propane-1,2,3-triyl triacetate (2) as a light brown gel. Yield: 1.0 g; LC-MS (ESI) m/z 550.48 [M+1]⁺.

### Synthesis of (1S,2R)-1-((2R,3R,4S,6S)-4-acetoxy-3-(2-acetoxyacetamido)-6-(acetylthio)-6-(methoxycarbonyl)tetrahydro-2H-pyran-2-yl)propane-1,2,3-triyl triacetate (3)

In an inert atmosphere, a solution of crude (1S,2R)-1-((2R,3R,4S,6R)-4-acetoxy-3-(2-acetoxyacetamido)-6-chloro-6-(methoxycarbonyl)tetrahydro-2H-pyran-2-yl)propane-1,2,3-triyl triacetate (2, 1.0 g, 1.76 mmol) in dry acetone (20.0 mL) was stirred at 0° C. To this solution, potassium thioacetate (0.603 g, 5.28 mmol) was added portion-wise at 0° C.; the reaction was stirred for 3 h at 0° C. and progress was monitored by TLC. After completion, the mixture was concentrated under reduced pressure to obtain a crude residue. The crude residue was dissolved in ethyl acetate and the resulting solution was washed with 1N HCl followed by DM water. The organic layer was separated, dried over anhydrous sodium sulfate, dried over sodium sulfate, and concentrated on a rotary evaporator to obtain a thick residue. The residue was purified by column chromatography (70-80% ethyl acetate in hexanes) to obtain (1S,2R)-1-((2R,3R,4S,6S)-4-acetoxy-3-(2-acetoxyacetamido)-6-(acetylthio)-6-(methoxycarbonyl)tetrahydro-2H-pyran-2-yl)propane-1,2,3-triyl triacetate (3) as a white solid. Yield: 0.780 g, 72.91%; LCMS (ESI) m/z 685.8 [M+1]⁺.

### Synthesis of (2S,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid (Cpd. No. 26729)

To a stirred solution of (1S,2R)-1-((2R,3R,4S,6S)-4-acetoxy-3-(2-acetoxyacetamido)-6-(acetylthio)-6-(methoxycarbonyl)tetrahydro-2H-pyran-2-yl)propane-1,2,3-triyl triacetate (3, 0.78 g, 1.28 mmol) in methanol (10.0 mL) was added sodium thiomethoxide (0.107 g, 1.54 mmol) at 0° C. The resulting mixture was stirred for 1 h. To the reaction, 1-iodo-2-[2-(prop-2-yn-1-yloxy)ethoxy]ethane (3a, 0.652 g, 2.57 mmol) was added at 0° C. and the reaction mixture was stirred at room temperature for 1 h. The reaction was monitored by LCMS. After completion, to the same reaction mass was added lithium hydroxide (0.092 g, 3.85 mmol) at room temperature and the reaction was stirred at room temperature for 6 h. The progress of the reaction was monitored by LC-MS and after completion, Dowex-hydrogen form was added up to pH 6 and the reaction mass was filtered through a sintered funnel. The filtrate was concentrated on a rotary evaporator to obtain a thick residue that was then purified via preparative HPLC (20-45% acetonitrile in water with 0.1% TFA). Fractions containing the desired product were combined and lyophilized to dryness to afford (2S,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid (Cpd. No. 26729) as a yellow semi-solid. Yield: 0.140 g, 23.33%; ELSD-MS (ESI) m/z 468.2 [M+1]⁺. ¹H NMR (400 MHz, methanol-d₄) δ 7.96 (d, J=7.6 Hz, 1H), 4.18 (d, J=2.4 H z, 1H), 4.02 (s, 2H), 3.90-3.81 (m, 4H), 3.72-3.57 (m, 8H), 3.52-3.50 (m, 1H), 3.34 (s, 1H), 2.97-2.77 (m, 4H), 1.82-1.76 (t, J=10.8 Hz, 1H).

### Example 5: Synthesis of Additional Conjugatable Siglec Ligands

### (2R,4R,5S,6S)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)-6-((1S,2S)-1,2,3-trihydroxypropyl)tetrahydro-2H-pyran-2-carboxylic acid (Example 1)

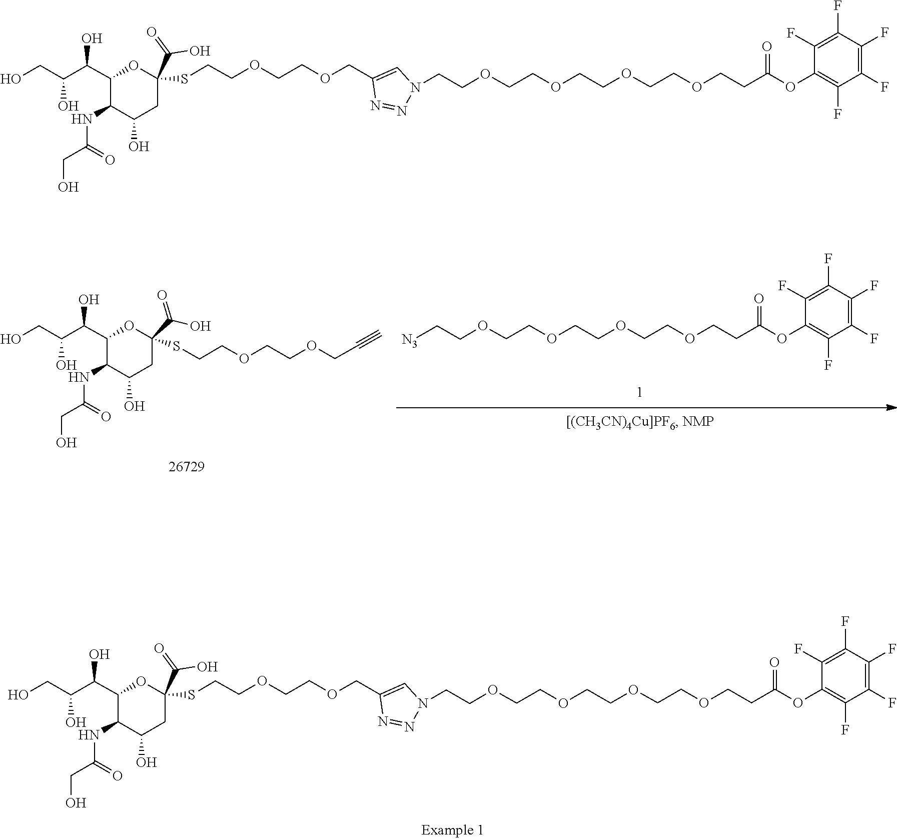

### Synthesis of (2R,4R,5S,6S)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)-6-((1S,2S)-1,2,3-trihydroxypropyl)tetrahydro-2H-pyran-2-carboxylic acid (Example 1)

To a stirred solution of (2R,4R,5S,6S)-4-hydroxy-5-(2-hydroxyacetamido)-2-((2-(2-(prop-2-yn-1-yloxy)ethoxy)ethyl)thio)-6-((1S,2S)-1,2,3-trihydroxypropyl)tetrahydro-2H-pyran-2-carboxylic acid (26729, 1.00 eq) in NMP (0.3 mL) in a 1 dram vial is added a solution of perfluorophenyl 1-azido-3,6,9,12-tetraoxapentadecan-15-oate (1, 1.10 eq) in NMP (0.3 mL) followed by tetrakis(acetonitrile)copper(I) hexafluorophosphate (2.50 eq). The resulting solution is capped and stirred at room temperature for 10 min. The reaction is diluted with acetic acid, filtered, and purified via preparatory HPLC (15-65% acetonitrile in water with 0.1% TFA). Fractions containing the desired product are combined and lyophilized to dryness to afford (2R,4R,5S,6S)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)-6-((1S,2S)-1,2,3-trihydroxypropyl)tetrahydro-2H-pyran-2-carboxylic acid (Example 1).

### (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(4-hydroxy-5-(2-hydroxyacetamido)-6-((1S,2S)-1,2,3-trihydroxypropyl)tetrahydro-2H-pyran-2-carboxylic acid) (Example 2)

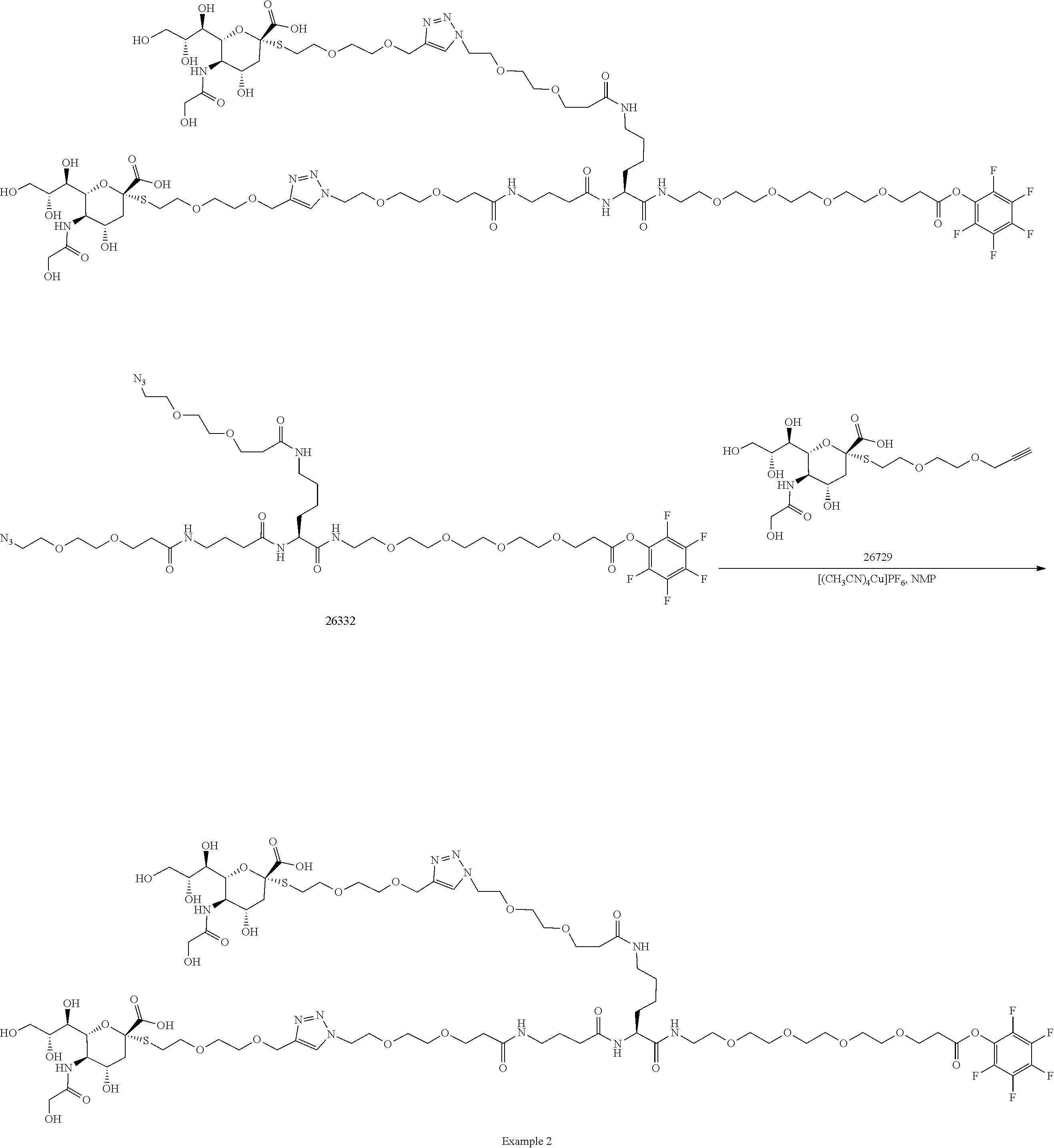

### Synthesis of (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(m eth yl ene))bis(oxy))bis(ethane-2,1-diyl))bis(oxy))bis(ethane-2,1-diyl))bis(sulfanediyl))bis(4-hydroxy-5-(2-hydroxyacetamido)-6-((1S,2S)-1,2,3-trihydroxypropyl)tetrahydro-2H-pyran-2-carboxylic acid) (Example 2)

To a stirred solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq) and (2R,4R,5S,6S)-4-hydroxy-5-(2-hydroxyacetamido)-2-((2-(2-(prop-2-yn-1-yloxy)ethoxy)ethyl)thio)-6-((1S,2S)-1,2,3-trihydroxypropyl)tetrahydro-2H-pyran-2-carboxylic acid (26729, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(4-hydroxy-5-(2-hydroxyacetamido)-6-((1S,2S)-1,2,3-trihydroxypropyl)tetrahydro-2H-pyran-2-carboxylic acid) (Example 2).

### Example 3

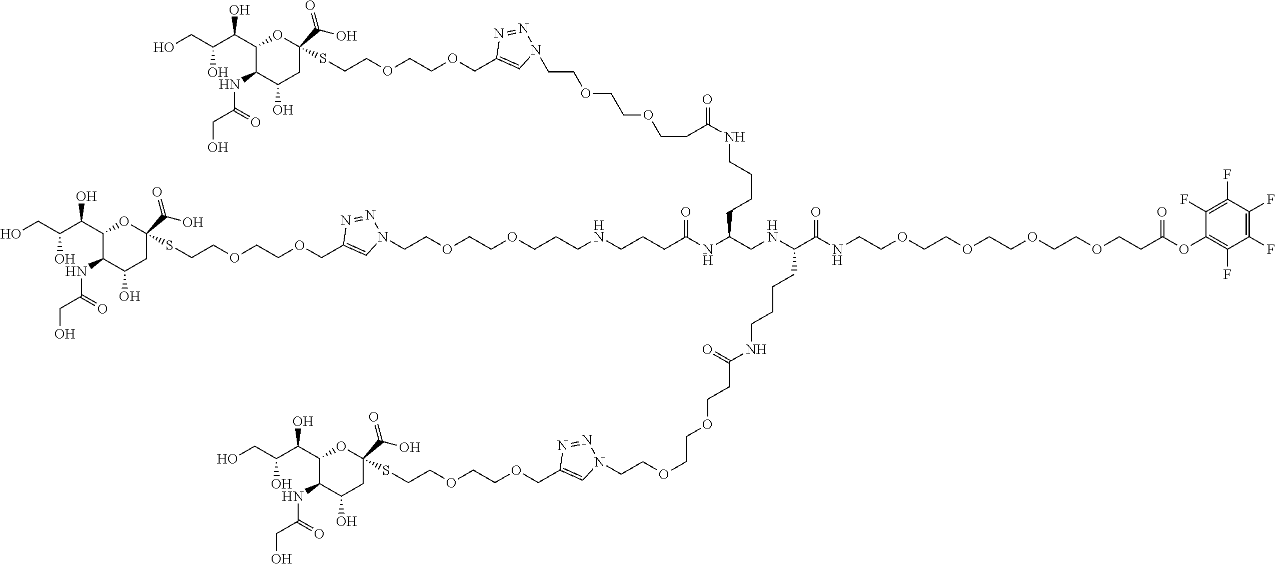 [SMILES: O=C(CCOCCOCCn1cc(COCCOCCS[C@]2(C(=O)O)C[C@H](O)[C@@H](NC(=O)CO)[C@H]([C@H](O)[C@H](O)CO)O2)nn1)NCCCC[C@@H](CN[C@@H](CCCCNC(=O)CCOCCOCCn1cc(COCCOCCS[C@]2(C(=O)O)C[C@H](O)[C@@H](NC(=O)CO)[C@H]([C@H](O)[C@H](O)CO)O2)nn1)C(=O)NCCOCCOCCOCCOCCC(=O)Oc1c(F)c(F)c(F)c(F)c1F)NC(=O)CCCNCCCOCCOCCn1cc(COCCOCCS[C@]2(C(=O)O)C[C@H](O)[C@@H](NC(=O)CO)[C@H]([C@H](O)[C@H](O)CO)O2)nn1]

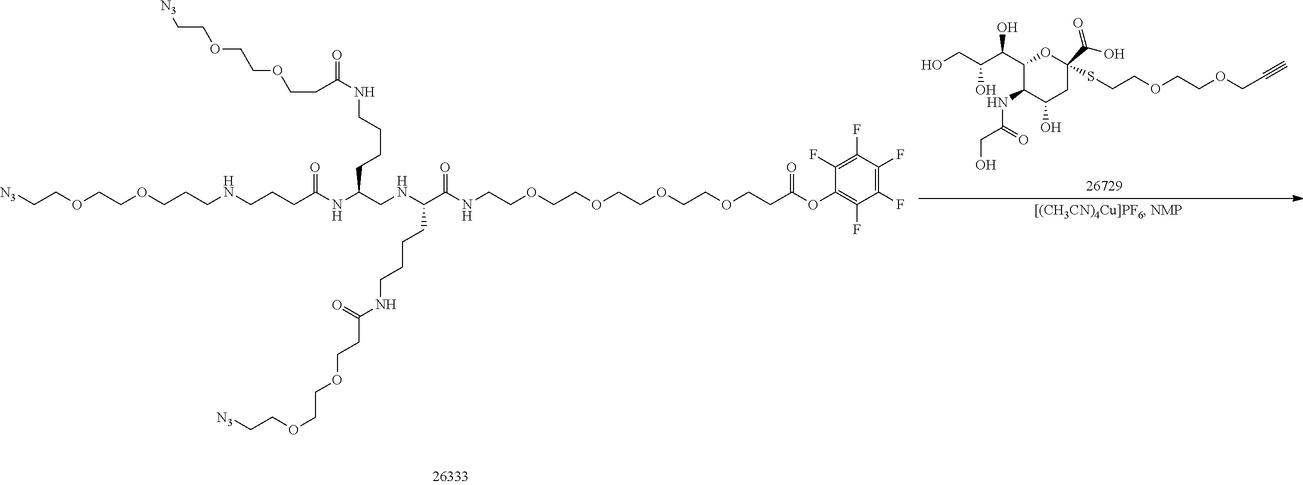 [SMILES: [N-]=[N+]=NCCOCCOCCCNCCCC(=O)N[C@@H](CCCCNC(=O)CCOCCOCCN=[N+]=[N-])CN[C@@H](CCCCNC(=O)CCOCCOCCN=[N+]=[N-])C(=O)NCCOCCOCCOCCOCCC(=O)Oc1c(F)c(F)c(F)c(F)c1F ; C#CCOCCOCCS[C@]1(C(=O)O)C[C@H](O)[C@@H](NC(=O)CO)[C@H]([C@H](O)[C@H](O)CO)O1]

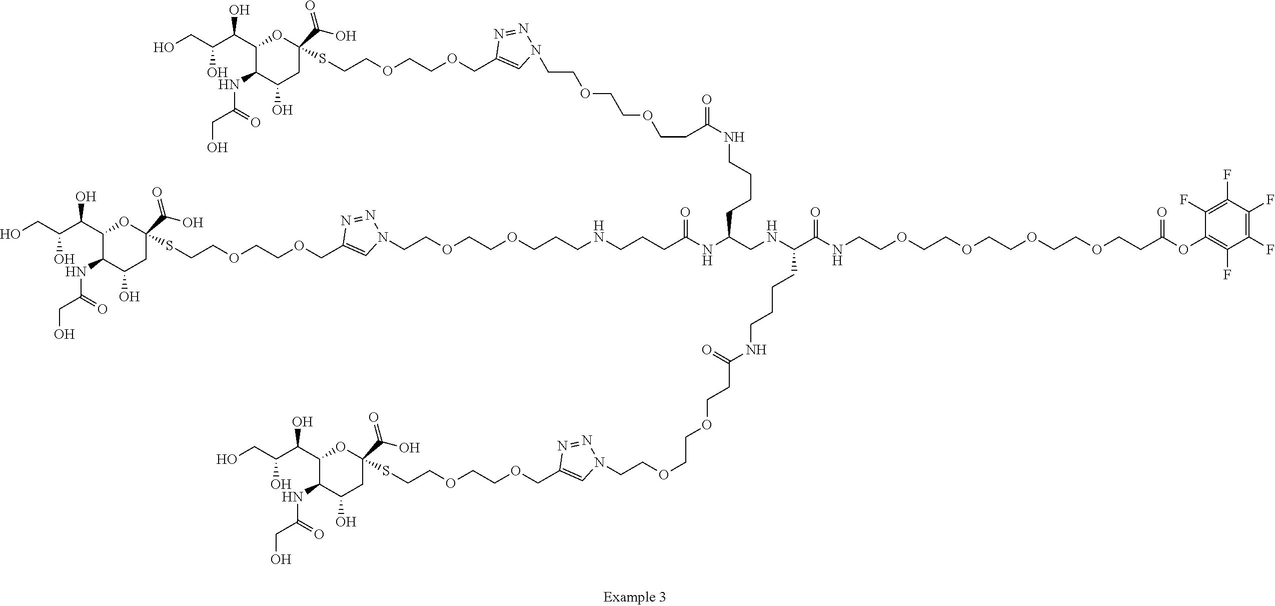 [SMILES: O=C(CCOCCOCCn1cc(COCCOCCS[C@]2(C(=O)O)C[C@H](O)[C@@H](NC(=O)CO)[C@H]([C@H](O)[C@H](O)CO)O2)nn1)NCCCC[C@@H](CN[C@@H](CCCCNC(=O)CCOCCOCCn1cc(COCCOCCS[C@]2(C(=O)O)C[C@H](O)[C@@H](NC(=O)CO)[C@H]([C@H](O)[C@H](O)CO)O2)nn1)C(=O)NCCOCCOCCOCCOCCC(=O)Oc1c(F)c(F)c(F)c(F)c1F)NC(=O)CCCNCCCOCCOCCn1cc(COCCOCCS[C@]2(C(=O)O)C[C@H](O)[C@@H](NC(=O)CO)[C@H]([C@H](O)[C@H](O)CO)O2)nn1]

### Synthesis of Example 3

To a stirred solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq) and (2R,4R,5S,6S)-4-hydroxy-5-(2-hydroxyacetamido)-2-((2-(2-(prop-2-yn-1-yloxy)ethoxy)ethyl)thio)-6-((1S,2S)-1,2,3-trihydroxypropyl)tetrahydro-2H-pyran-2-carboxylic acid (26729, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford Example 3.

### (2R,4S,5R,6R)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)-6-((1R,2R)-1,2,3-trihydroxypropyl)tetrahydro-2H-pyran-2-carboxylic acid (Example 4)

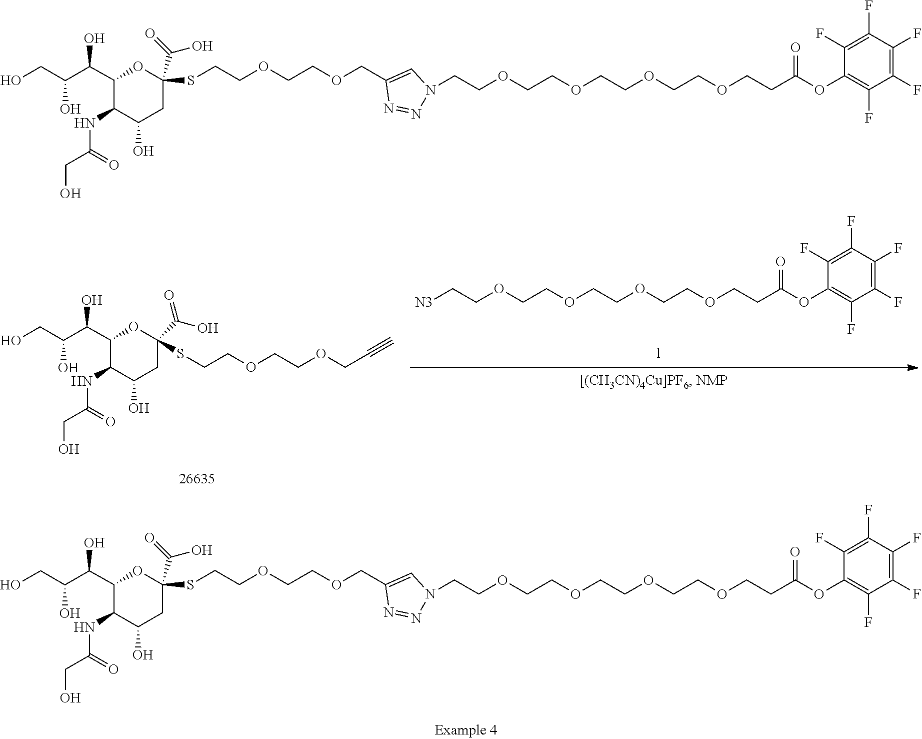 [SMILES: O=C(CO)N[C@H]1[C@H]([C@H](O)[C@H](O)CO)O[C@](SCCOCCOCc2cn(CCOCCOCCOCCOCCC(=O)Oc3c(F)c(F)c(F)c(F)c3F)nn2)(C(=O)O)C[C@@H]1O ; C#CCOCCOCCS[C@@]1(C(=O)O)C[C@H](O)[C@@H](NC(=O)CO)[C@H]([C@H](O)[C@H](O)CO)O1 ; [N-]=[N+]=NCCOCCOCCOCCOCCC(=O)Oc1c(F)c(F)c(F)c(F)c1F ; O=C(CO)N[C@H]1[C@H]([C@H](O)[C@H](O)CO)O[C@](SCCOCCOCc2cn(CCOCCOCCOCCOCCC(=O)Oc3c(F)c(F)c(F)c(F)c3F)nn2)(C(=O)O)C[C@@H]1O]

### Synthesis of (2R,4S,5R,6R)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)-6-((1R,2R)-1,2,3-trihydroxypropyl)tetrahydro-2H-pyran-2-carboxylic acid (Example 4)

To a stirred solution of (2R,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid (26635, 1.00 eq) in NMP (0.3 mL) in a 1 dram vial is added a solution of perfluorophenyl 1-azido-3,6,9,12-tetraoxapentadecan-15-oate (1, 1.10 eq) in NMP (0.3 mL) followed by tetrakis(acetonitrile)copper(I) hexafluorophosphate (2.50 eq). The resulting solution is capped and stirred at room temperature for 10 min. The reaction is diluted with acetic acid, filtered, and purified via preparatory HPLC (15-65% acetonitrile in water with 0.1% TFA). Fractions containing the desired product are combined and lyophilized to dryness to afford (2R,4S,5R,6R)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)-6-((1R,2R)-1,2,3-trihydroxypropyl)tetrahydro-2H-pyran-2-carboxylic acid (Example 4).

### (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(4-hydroxy-5-(2-hydroxyacetamido)-6-((1R,2R)-1,2,3-trihydroxypropyl)tetrahydro-2H-pyran-2-carboxylic acid) (Example 5)

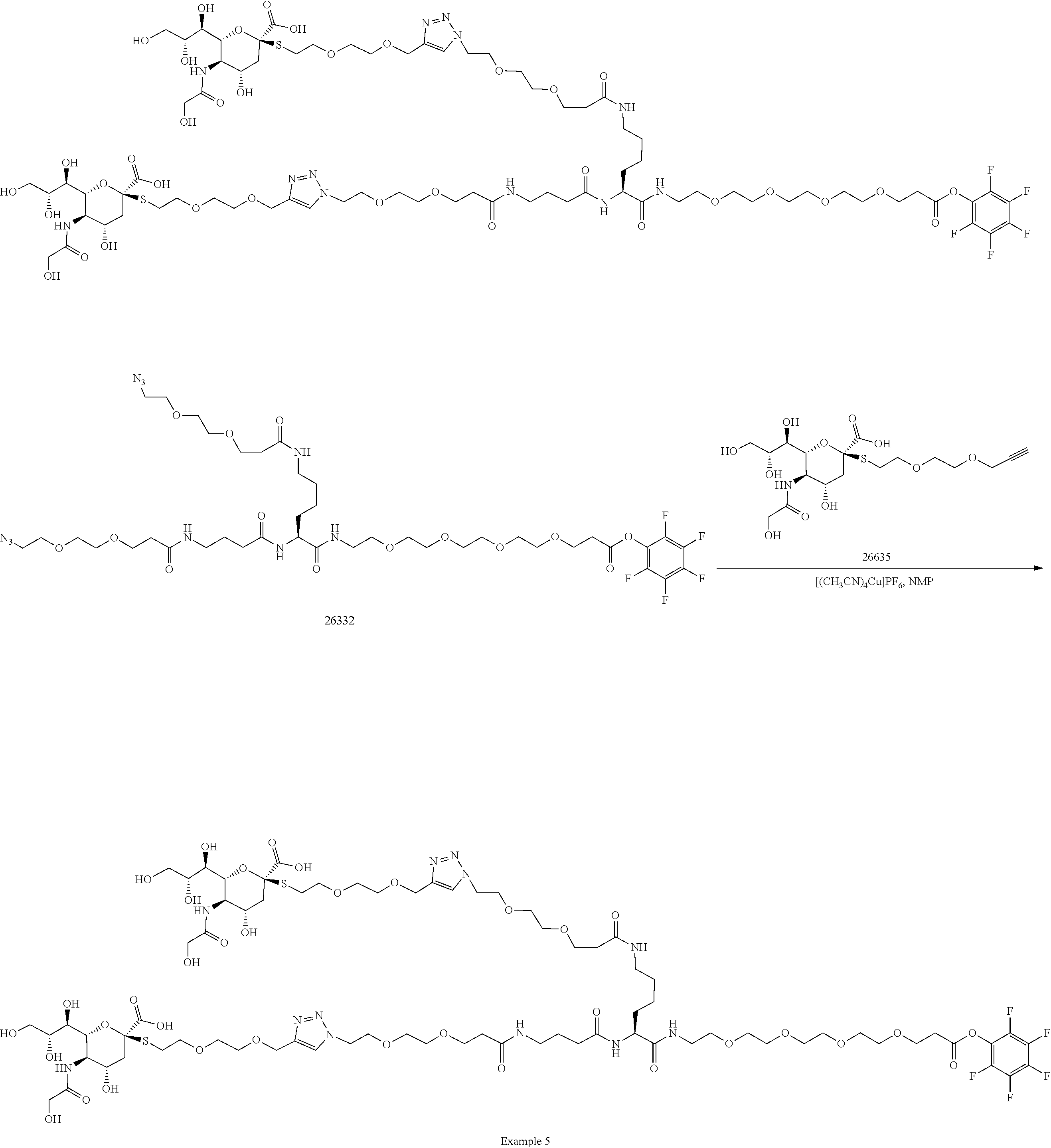

### Synthesis of (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(4-hydroxy-5-(2-hydroxyacetamido)-6-((1R,2R)-1,2,3-trihydroxypropyl)tetrahydro-2H-pyran-2-carboxylic acid) (Example 5)

To a stirred solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq) and (2R,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid (26635, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(4-hydroxy-5-(2-hydroxyacetamido)-6-((1R,2R)-1,2,3-trihydroxypropyl)tetrahydro-2H-pyran-2-carboxylic acid) (Example 5).

### Example 6

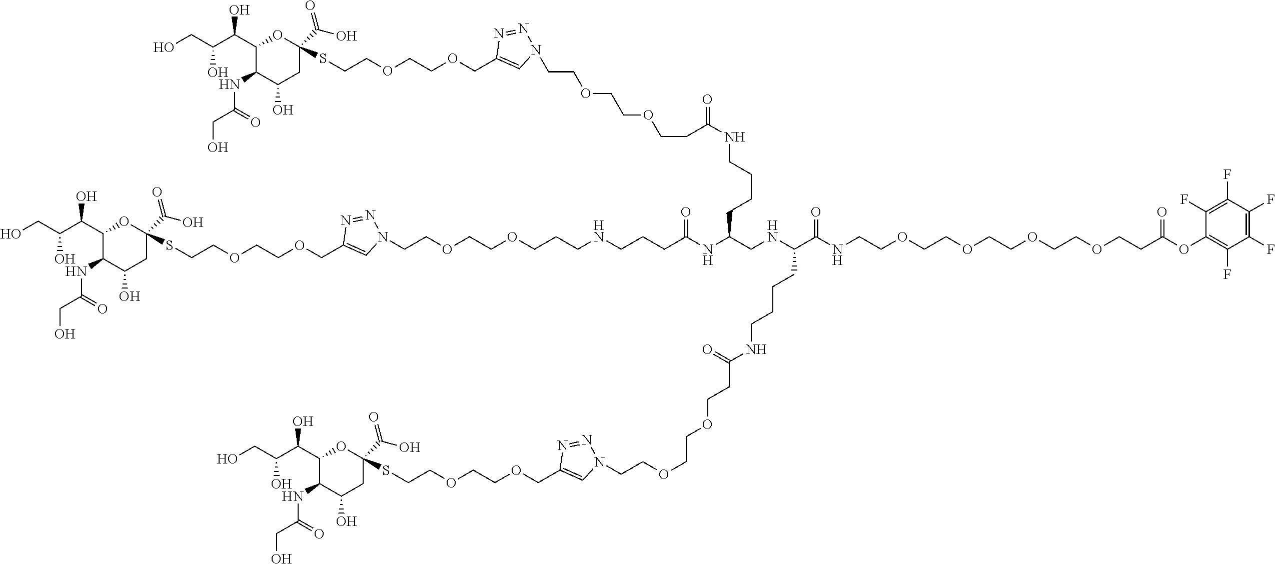 [SMILES: O=C(CCOCCOCCn1cc(COCCOCCS[C@@]2(C(=O)O)C[C@H](O)[C@@H](NC(=O)CO)[C@H]([C@H](O)[C@H](O)CO)O2)nn1)NCCCC[C@@H](CN[C@@H](CCCCNC(=O)CCOCCOCCn1cc(COCCOCCS[C@@]2(C(=O)O)C[C@H](O)[C@@H](NC(=O)CO)[C@H]([C@H](O)[C@H](O)CO)O2)nn1)C(=O)NCCOCCOCCOCCOCCC(=O)Oc1c(F)c(F)c(F)c(F)c1F)NC(=O)CCCNCCCOCCOCCn1cc(COCCOCCS[C@@]2(C(=O)O)C[C@H](O)[C@@H](NC(=O)CO)[C@H]([C@H](O)[C@H](O)CO)O2)nn1]

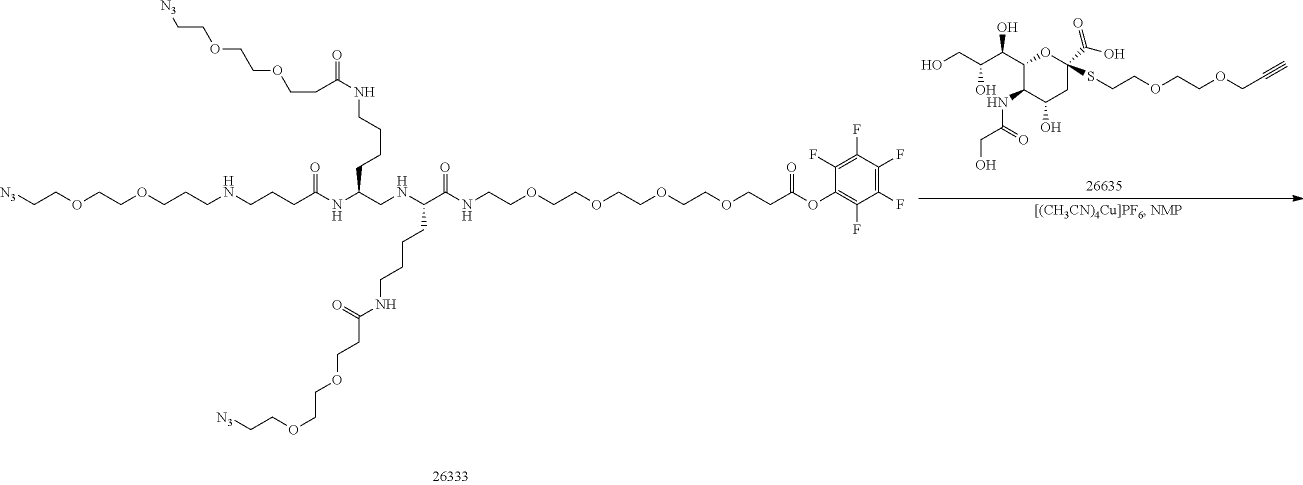 [SMILES: [N-]=[N+]=NCCOCCOCCCNCCCC(=O)N[C@@H](CCCCNC(=O)CCOCCOCCN=[N+]=[N-])CN[C@@H](CCCCNC(=O)CCOCCOCCN=[N+]=[N-])C(=O)NCCOCCOCCOCCOCCC(=O)Oc1c(F)c(F)c(F)c(F)c1F ; C#CCOCCOCCS[C@@]1(C(=O)O)C[C@H](O)[C@@H](NC(=O)CO)[C@H]([C@H](O)[C@H](O)CO)O1]

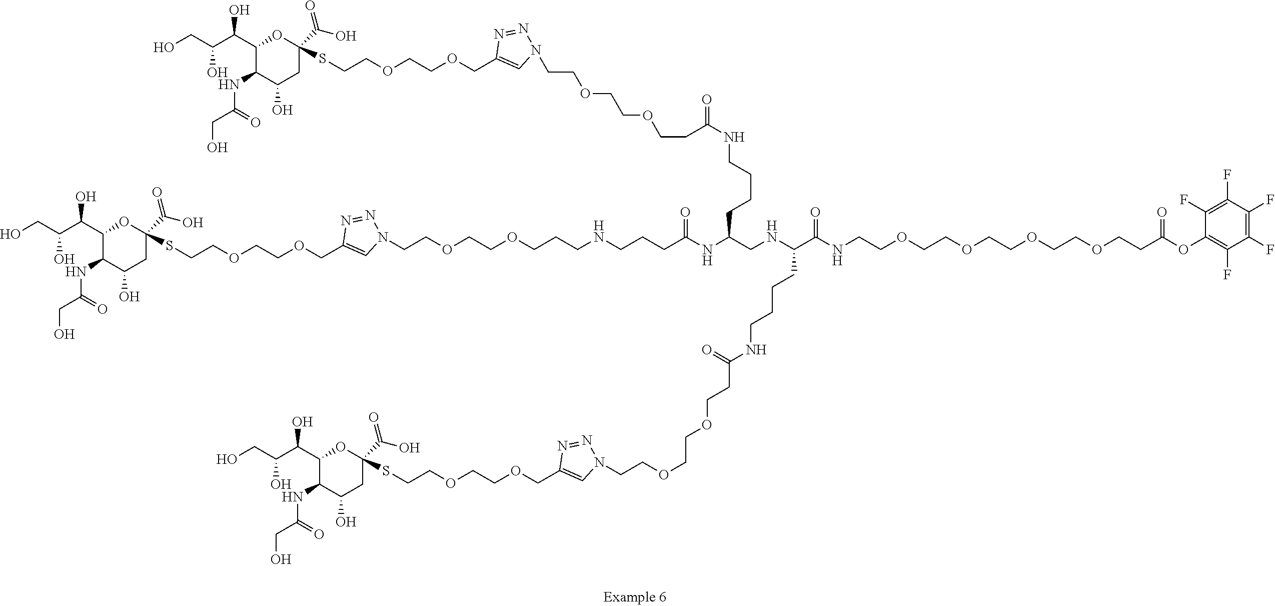 [SMILES: O=C(CCOCCOCCn1cc(COCCOCCS[C@@]2(C(=O)O)C[C@H](O)[C@@H](NC(=O)CO)[C@H]([C@H](O)[C@H](O)CO)O2)nn1)NCCCC[C@@H](CN[C@@H](CCCCNC(=O)CCOCCOCCn1cc(COCCOCCS[C@@]2(C(=O)O)C[C@H](O)[C@@H](NC(=O)CO)[C@H]([C@H](O)[C@H](O)CO)O2)nn1)C(=O)NCCOCCOCCOCCOCCC(=O)Oc1c(F)c(F)c(F)c(F)c1F)NC(=O)CCCNCCCOCCOCCn1cc(COCCOCCS[C@@]2(C(=O)O)C[C@H](O)[C@@H](NC(=O)CO)[C@H]([C@H](O)[C@H](O)CO)O2)nn1]

### Synthesis of Example 6

To a stirred solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq) and (2R,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid (26635, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford Example 6.

### (2RS,4RS,5SR,6SR)-4-hydroxy-5-(2-hydroxyacetamido)-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)-6-((1S R,2SR)-1,2,3-trihydroxypropyl)tetrahydro-2H-pyran-2-carboxylic acid (Example 7)

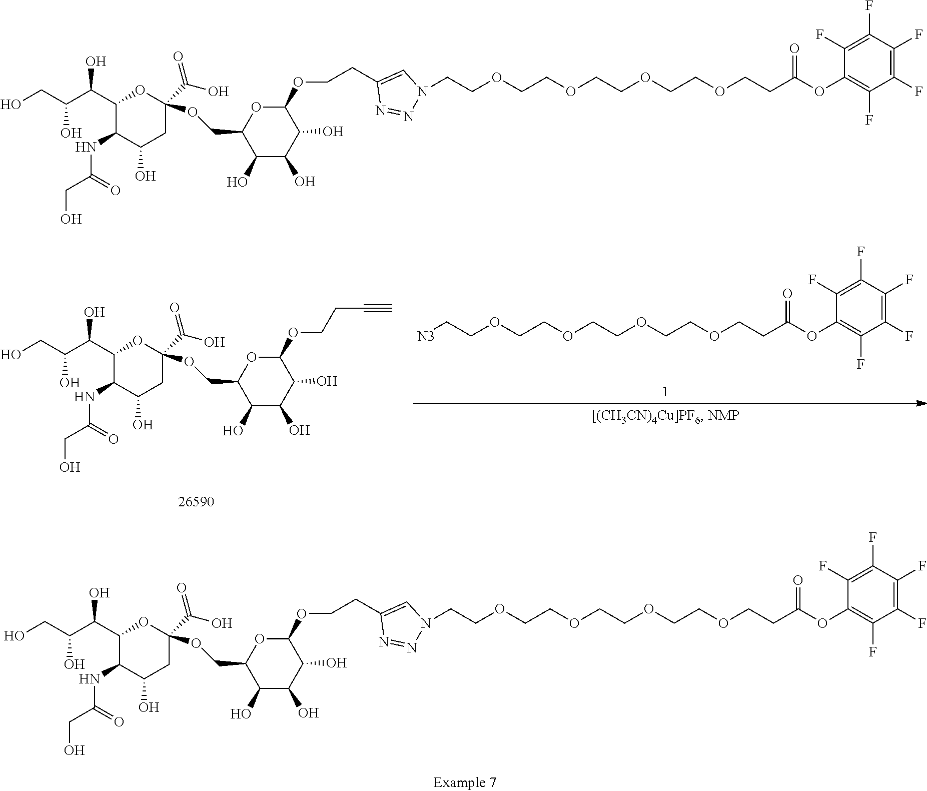 [SMILES: O=C(CO)N[C@H]1[C@H]([C@H](O)[C@H](O)CO)O[C@](OC[C@H]2O[C@@H](OCCc3cn(CCOCCOCCOCCOCCC(=O)Oc4c(F)c(F)c(F)c(F)c4F)nn3)[C@H](O)[C@@H](O)[C@H]2O)(C(=O)O)C[C@@H]1O ; C#CCCO[C@@H]1O[C@H](CO[C@@]2(C(=O)O)C[C@H](O)[C@@H](NC(=O)CO)[C@H]([C@H](O)[C@H](O)CO)O2)[C@H](O)[C@H](O)[C@H]1O ; [N-]=[N+]=NCCOCCOCCOCCOCCC(=O)Oc1c(F)c(F)c(F)c(F)c1F ; O=C(CO)N[C@H]1[C@H]([C@H](O)[C@H](O)CO)O[C@](OC[C@H]2O[C@@H](OCCc3cn(CCOCCOCCOCCOCCC(=O)Oc4c(F)c(F)c(F)c(F)c4F)nn3)[C@H](O)[C@@H](O)[C@H]2O)(C(=O)O)C[C@@H]1O]

### Synthesis of (2RS,4RS,5SR,6SR)-4-hydroxy-5-(2-hydroxyacetamido)-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)-6-((1SR,2SR)-1,2,3-trihydroxypropyl)tetrahydro-2H-pyran-2-carboxylic acid (Example 7)

To a stirred solution of (2RS,4RS,5SR,6SR)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)-6-((1SR,2SR)-1,2,3-trihydroxypropyl)tetrahydro-2H-pyran-2-carboxylic acid (26590, 1.00 eq) in NMP (0.3 mL) in a 1 dram vial is added a solution of perfluorophenyl 1-azido-3,6,9,12-tetraoxapentadecan-15-oate (1, 1.10 eq) in NMP (0.3 mL) followed by tetrakis(acetonitrile)copper(I) hexafluorophosphate (2.50 eq). The resulting solution is capped and stirred at room temperature for 10 min. The reaction is diluted with acetic acid, filtered, and purified via preparatory HPLC (15-65% acetonitrile in water with 0.1% TFA). Fractions containing the desired product are combined and lyophilized to dryness to afford (2RS,4RS,5SR,6SR)-4-hydroxy-5-(2-hydroxyacetamido)-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)-6-((1SR,2SR)-1,2,3-trihydroxypropyl)tetrahydro-2H-pyran-2-carboxylic acid (Example 7).

### (2RS,2′RS,4RS,4′RS,5SR,5′SR,6SR,6′SR)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(4-hydroxy-5-(2-hydroxyacetamido)-6-((1SR,2SR)-1,2,3-trihydroxypropyl)tetrahydro-2H-pyran-2-carboxylic acid) (Example 8)

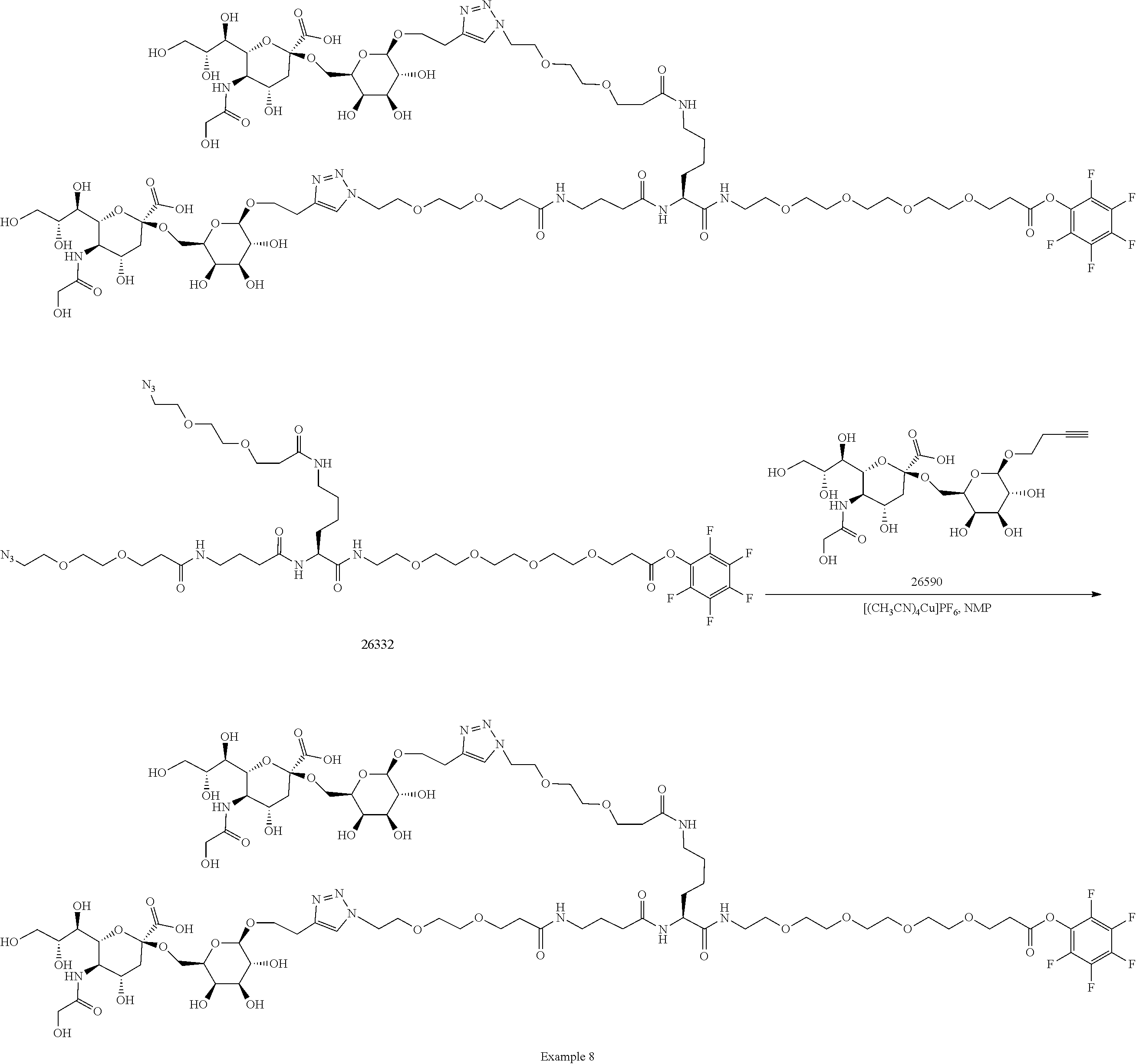

### Synthesis of (2RS,2′RS,4RS,4′RS,5SR,5′SR,6SR,6′SR)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(meth yl ene))bis(oxy))bis(4-hydroxy-5-(2-hydroxyacetamido)-6-((1SR,2SR)-1, 2, 3-trihydroxypropyl)tetrahydro-2H-pyran-2-carboxylic acid) (Example 8)

To a stirred solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq) and (2RS,4RS,5SR,6SR)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)-6-((1SR,2SR)-1,2,3-trihydroxypropyl)tetrahydro-2H-pyran-2-carboxylic acid (26590, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2RS,2′RS,4RS,4′RS,5SR,5′SR,6SR,6′SR)-2,2′-((((2R,2′R,3R,3′R,4S,4′S,5R,5′R,6R,6′R)—(((((RS)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(ethane-2,1-diyl))bis(oxy))bis(3,4,5-trihydroxytetrahydro-2H-pyran-6,2-diyl))bis(methylene))bis(oxy))bis(4-hydroxy-5-(2-hydroxyacetamido)-6-((1SR,2SR)-1,2,3-trihydroxypropyl)tetrahydro-2H-pyran-2-carboxylic acid) (Example 8).

### Example 9

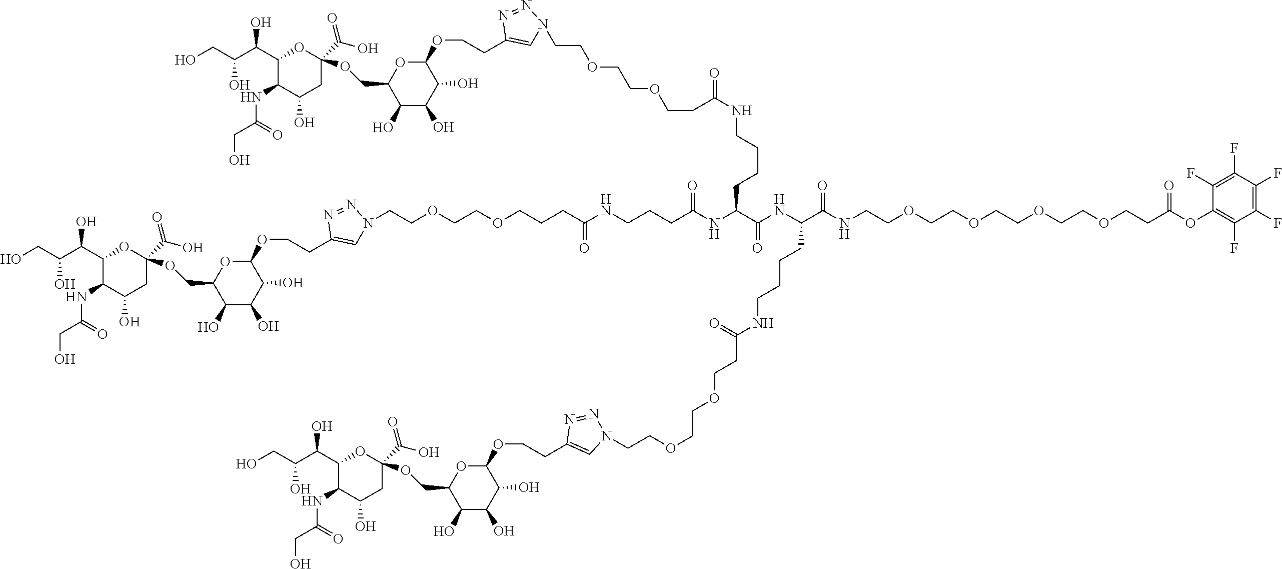 [SMILES: O=C(CCCOCCOCCn1cc(CCO[C@@H]2O[C@H](CO[C@@]3(C(=O)O)C[C@H](O)[C@@H](NC(=O)CO)[C@H]([C@H](O)[C@H](O)CO)O3)[C@H](O)[C@H](O)[C@H]2O)nn1)NCCCC(=O)N[C@@H](CCCCNC(=O)CCOCCOCCn1cc(CCO[C@@H]2O[C@H](CO[C@@]3(C(=O)O)C[C@H](O)[C@@H](NC(=O)CO)[C@H]([C@H](O)[C@H](O)CO)O3)[C@H](O)[C@H](O)[C@H]2O)nn1)C(=O)N[C@@H](CCCCNC(=O)CCOCCOCCn1cc(CCO[C@@H]2O[C@H](CO[C@@]3(C(=O)O)C[C@H](O)[C@@H](NC(=O)CO)[C@H]([C@H](O)[C@H](O)CO)O3)[C@H](O)[C@H](O)[C@H]2O)nn1)C(=O)NCCOCCOCCOCCOCCC(=O)Oc1c(F)c(F)c(F)c(F)c1F]

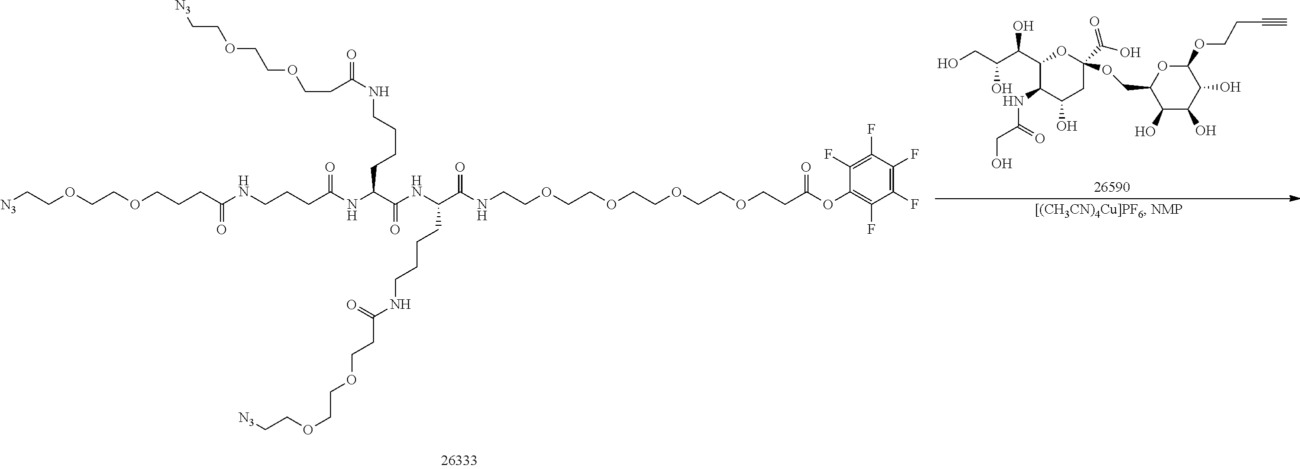 [SMILES: [N-]=[N+]=NCCOCCOCCCC(=O)NCCCC(=O)N[C@@H](CCCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F ; C#CCCO[C@@H]1O[C@H](CO[C@@]2(C(=O)O)C[C@H](O)[C@@H](NC(=O)CO)[C@H]([C@H](O)[C@H](O)CO)O2)[C@H](O)[C@H](O)[C@H]1O]

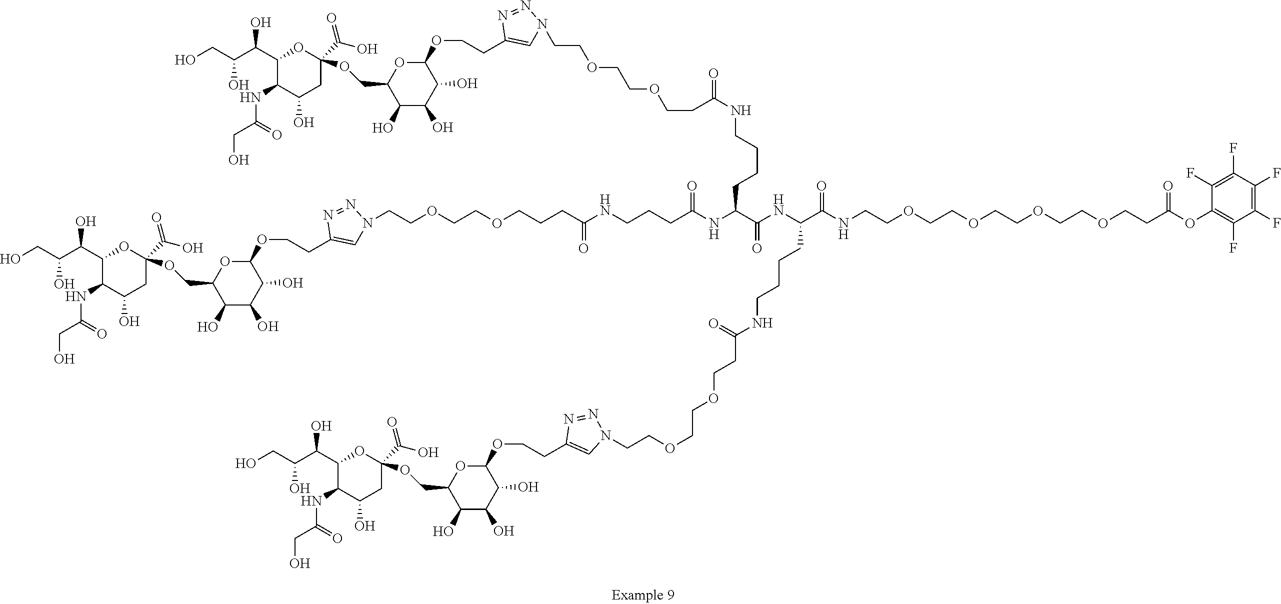 [SMILES: O=C(CCCOCCOCCn1cc(CCO[C@@H]2O[C@H](CO[C@@]3(C(=O)O)C[C@H](O)[C@@H](NC(=O)CO)[C@H]([C@H](O)[C@H](O)CO)O3)[C@H](O)[C@H](O)[C@H]2O)nn1)NCCCC(=O)N[C@@H](CCCCNC(=O)CCOCCOCCn1cc(CCO[C@@H]2O[C@H](CO[C@@]3(C(=O)O)C[C@H](O)[C@@H](NC(=O)CO)[C@H]([C@H](O)[C@H](O)CO)O3)[C@H](O)[C@H](O)[C@H]2O)nn1)C(=O)N[C@@H](CCCCNC(=O)CCOCCOCCn1cc(CCO[C@@H]2O[C@H](CO[C@@]3(C(=O)O)C[C@H](O)[C@@H](NC(=O)CO)[C@H]([C@H](O)[C@H](O)CO)O3)[C@H](O)[C@H](O)[C@H]2O)nn1)C(=O)NCCOCCOCCOCCOCCC(=O)Oc1c(F)c(F)c(F)c(F)c1F]

### Synthesis of Example 9

To a stirred solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq) and (2RS,4RS,5SR,6SR)-2-(((2R,3R,4S,5R,6R)-6-(but-3-yn-1-yloxy)-3,4,5-trihydroxytetrahydro-2H-pyran-2-yl)methoxy)-4-hydroxy-5-(2-hydroxyacetamido)-6-((1SR,2SR)-1,2,3-trihydroxypropyl)tetrahydro-2H-pyran-2-carboxylic acid (26590, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TEA). Fractions containing the desired product are combined and lyophilized to dryness to afford Example 9.

### (2R,4R,5S,6S)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)-6-((1S,2S)-1,2,3-trihydroxypropyl)tetrahydro-2H-pyran-2-carboxylic acid (Example 10)

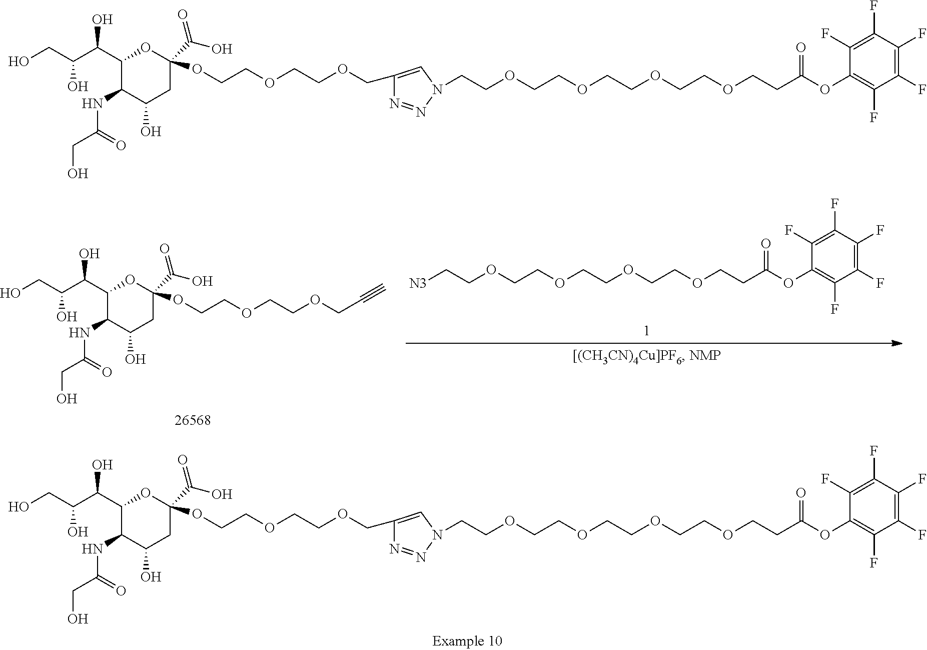

### Synthesis of (2R,4R,5S,6S)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)-6-((1S,2S)-1,2,3-trihydroxypropyl)tetrahydro-2H-pyran-2-carboxylic acid (Example 10)

To a stirred solution of (2R,4R,5S,6S)-4-hydroxy-5-(2-hydroxyacetamido)-2-(2-(2-(prop-2-yn-1-yloxy)ethoxy)ethoxy)-6-((1S,2S)-1,2,3-trihydroxypropyl)tetrahydro-2H-pyran-2-carboxylic acid (26568, 1.00 eq) in NMP (0.3 mL) in a 1 dram vial is added a solution of perfluorophenyl 1-azido-3,6,9,12-tetraoxapentadecan-15-oate (1, 1.10 eq) in NMP (0.3 mL) followed by tetrakis(acetonitrile)copper(I) hexafluorophosphate (2.50 eq). The resulting solution is capped and stirred at room temperature for 10 min. The reaction is diluted with acetic acid, filtered, and purified via preparatory HPLC (15-65% acetonitrile in water with 0.1% TFA). Fractions containing the desired product are combined and lyophilized to dryness to afford (2R,4R,5S,6S)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)-6-((1S,2S)-1,2,3-trihydroxypropyl)tetrahydro-2H-pyran-2-carboxylic acid (Example 10).

### (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(4-hydroxy-5-(2-hydroxyacetamido)-6-((1S,2S)-1,2,3-trihydroxypropyl)tetrahydro-2H-pyran-2-carboxylic acid) (Example 11)

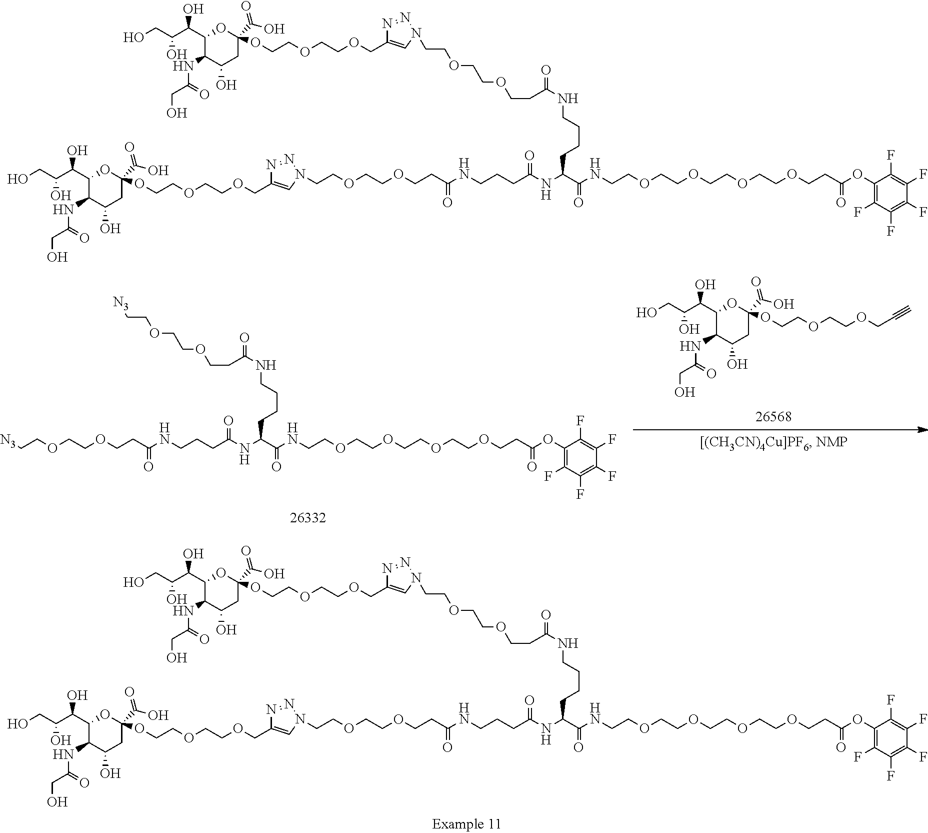

### (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(4-hydroxy-5-(2-hydroxyacetamido)-6-((1S,2S)-1,2,3-trihydroxypropyl)tetrahydro-2H-pyran-2-carboxylic acid) (Example 11)

To a stirred solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq) and (2R,4R,5S,6S)-4-hydroxy-5-(2-hydroxyacetamido)-2-(2-(2-(prop-2-yn-1-yloxy)ethoxy)ethoxy)-6-((1S,2S)-1,2,3-trihydroxypropyl)tetrahydro-2H-pyran-2-carboxylic acid (26568, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(4-hydroxy-5-(2-hydroxyacetamido)-6-((1S,2S)-1,2,3-trihydroxypropyl)tetrahydro-2H-pyran-2-carboxylic acid) (Example 11).

### Example 12

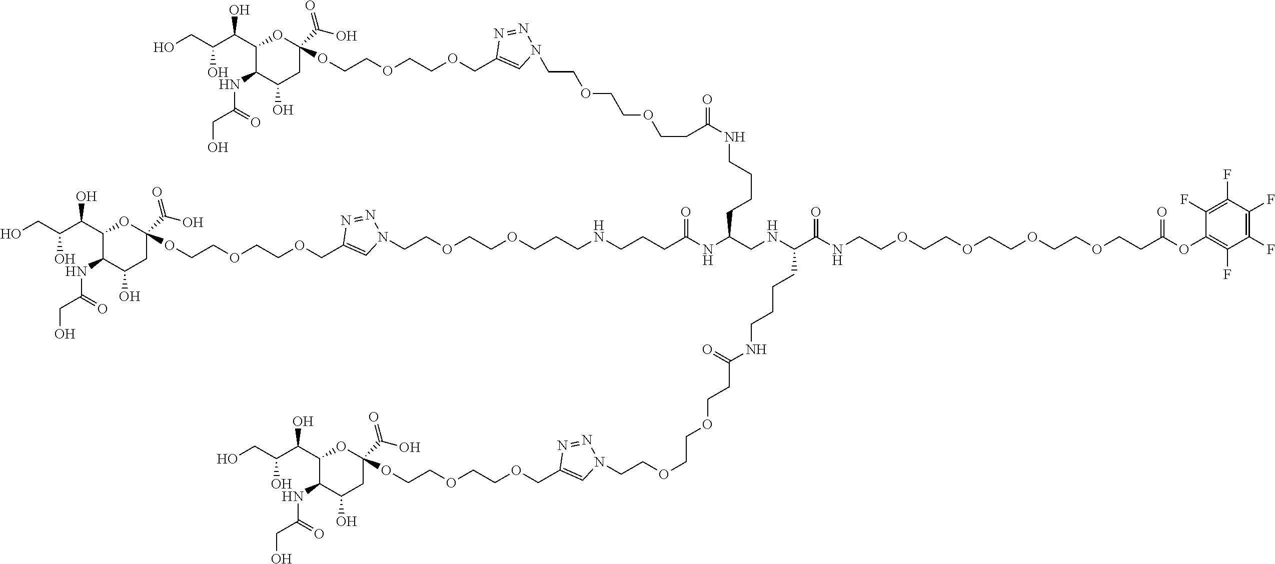 [SMILES: O=C(CCOCCOCCn1cc(COCCOCCO[C@@]2(C(=O)O)C[C@H](O)[C@@H](NC(=O)CO)[C@H]([C@H](O)[C@H](O)CO)O2)nn1)NCCCC[C@@H](CN[C@@H](CCCCNC(=O)CCOCCOCCn1cc(COCCOCCO[C@@]2(C(=O)O)C[C@H](O)[C@@H](NC(=O)CO)[C@H]([C@H](O)[C@H](O)CO)O2)nn1)C(=O)NCCOCCOCCOCCOCCC(=O)Oc1c(F)c(F)c(F)c(F)c1F)NC(=O)CCCNCCCOCCOCCn1cc(COCCOCCO[C@@]2(C(=O)O)C[C@H](O)[C@@H](NC(=O)CO)[C@H]([C@H](O)[C@H](O)CO)O2)nn1]

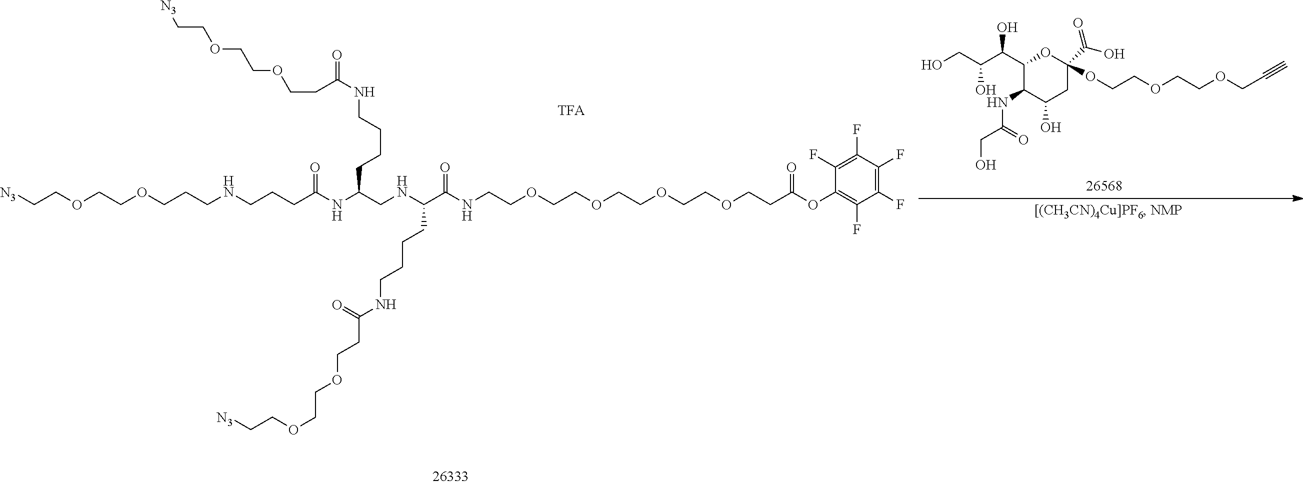 [SMILES: [N-]=[N+]=NCCOCCOCCCNCCCC(=O)N[C@@H](CCCCNC(=O)CCOCCOCCN=[N+]=[N-])CN[C@@H](CCCCNC(=O)CCOCCOCCN=[N+]=[N-])C(=O)NCCOCCOCCOCCOCCC(=O)Oc1c(F)c(F)c(F)c(F)c1F ; C#CCOCCOCCO[C@@]1(C(=O)O)C[C@H](O)[C@@H](NC(=O)CO)[C@H]([C@H](O)[C@H](O)CO)O1]

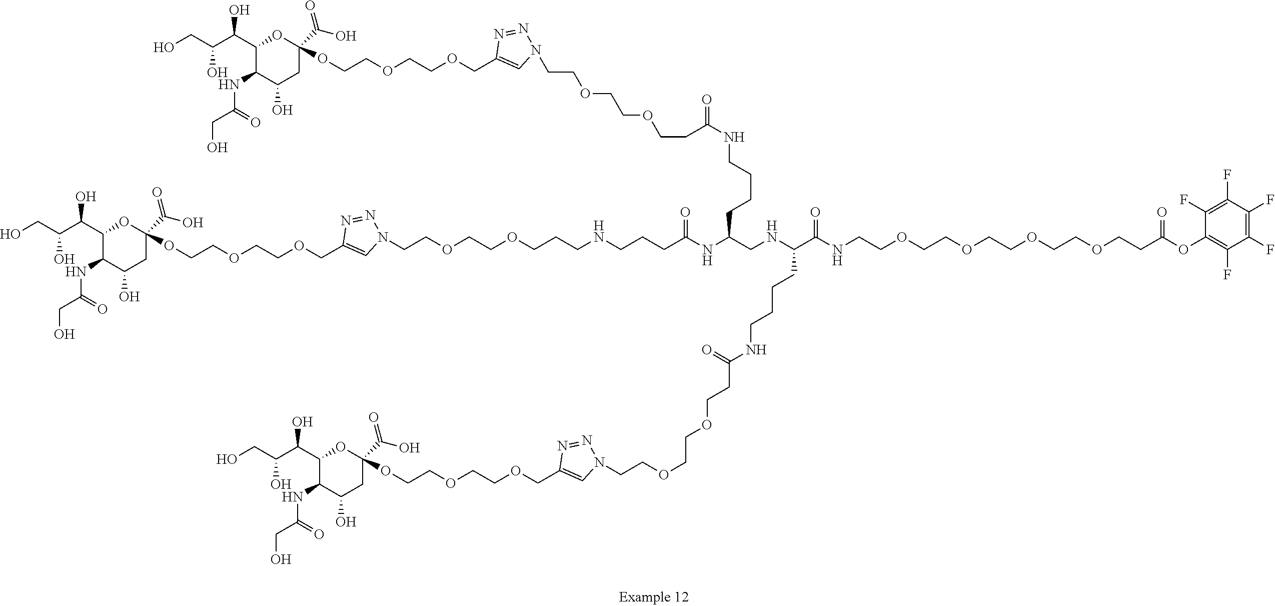 [SMILES: O=C(CCOCCOCCn1cc(COCCOCCO[C@@]2(C(=O)O)C[C@H](O)[C@@H](NC(=O)CO)[C@H]([C@H](O)[C@H](O)CO)O2)nn1)NCCCC[C@@H](CN[C@@H](CCCCNC(=O)CCOCCOCCn1cc(COCCOCCO[C@@]2(C(=O)O)C[C@H](O)[C@@H](NC(=O)CO)[C@H]([C@H](O)[C@H](O)CO)O2)nn1)C(=O)NCCOCCOCCOCCOCCC(=O)Oc1c(F)c(F)c(F)c(F)c1F)NC(=O)CCCNCCCOCCOCCn1cc(COCCOCCO[C@@]2(C(=O)O)C[C@H](O)[C@@H](NC(=O)CO)[C@H]([C@H](O)[C@H](O)CO)O2)nn1]

### Synthesis of Example 12

To a stirred solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq) and (2R,4R,5S,6S)-4-hydroxy-5-(2-hydroxyacetamido)-2-(2-(2-(prop-2-yn-1-yloxy)ethoxy)ethoxy)-6-((1S,2S)-1,2,3-trihydroxypropyl)tetrahydro-2H-pyran-2-carboxylic acid (26568, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford Example 12.

### (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)tetrahydro-2H-pyran-2-carboxylic acid (Example 13)

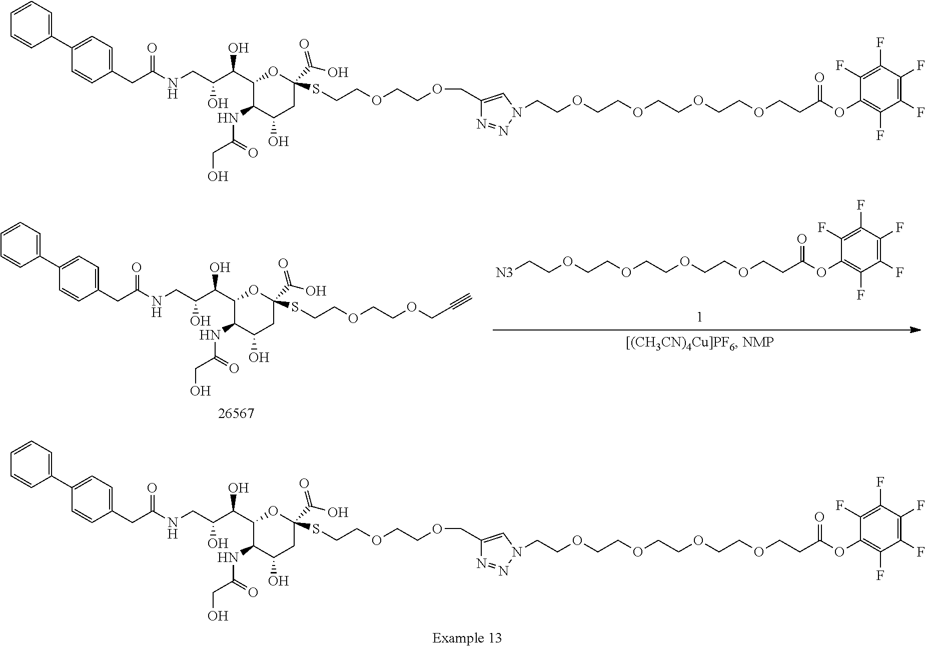 [SMILES: O=C(Cc1ccc(-c2ccccc2)cc1)NC[C@@H](O)[C@@H](O)[C@@H]1O[C@](SCCOCCOCc2cn(CCOCCOCCOCCOCCC(=O)Oc3c(F)c(F)c(F)c(F)c3F)nn2)(C(=O)O)C[C@H](O)[C@H]1NC(=O)CO ; C#CCOCCOCCS[C@@]1(C(=O)O)C[C@H](O)[C@@H](NC(=O)CO)[C@H]([C@H](O)[C@H](O)CNC(=O)Cc2ccc(-c3ccccc3)cc2)O1 ; [N-]=[N+]=NCCOCCOCCOCCOCCC(=O)Oc1c(F)c(F)c(F)c(F)c1F ; O=C(Cc1ccc(-c2ccccc2)cc1)NC[C@@H](O)[C@@H](O)[C@@H]1O[C@](SCCOCCOCc2cn(CCOCCOCCOCCOCCC(=O)Oc3c(F)c(F)c(F)c(F)c3F)nn2)(C(=O)O)C[C@H](O)[C@H]1NC(=O)CO]

### Synthesis of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)eth yl)thio)tetrahydro-2H-pyran-2-carboxylic acid (Example 13)

To a stirred solution of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (26567, 1.00 eq) in NMP (0.3 mL) in a 1 dram vial is added a solution of perfluorophenyl 1-azido-3,6,9,12-tetraoxapentadecan-15-oate (1, 1.10 eq) in NMP (0.3 mL) followed by tetrakis(acetonitrile)copper(I) hexafluorophosphate (2.50 eq). The resulting solution is capped and stirred at room temperature for 10 min. The reaction is diluted with acetic acid, filtered, and purified via preparatory HPLC (15-65% acetonitrile in water with 0.1% TFA). Fractions containing the desired product are combined and lyophilized to dryness to afford (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)tetrahydro-2H-pyran-2-carboxylic acid (Example 13).

### (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Example 14)

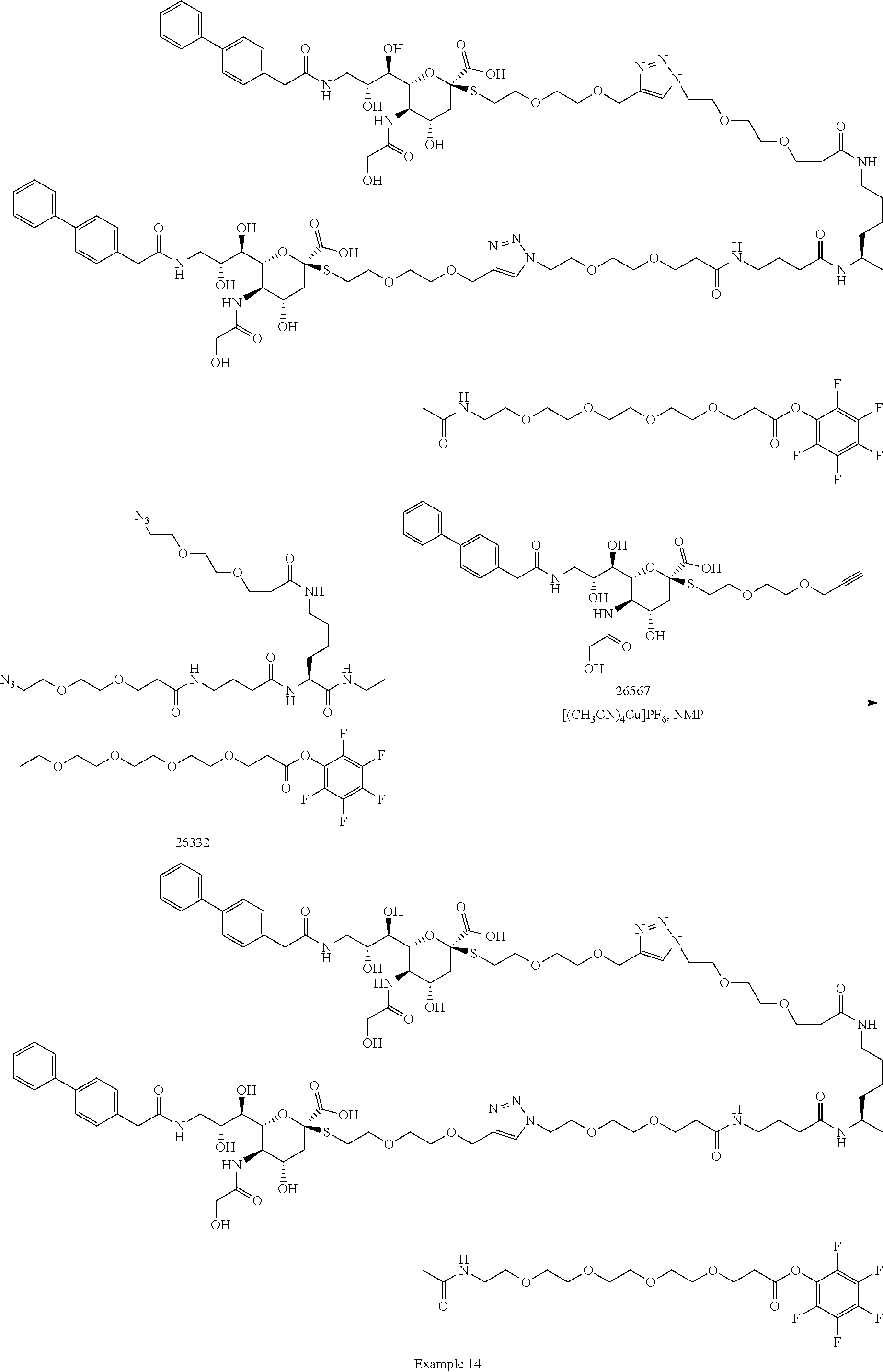

### Synthesis of (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Example 14)

To a stirred solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (26567, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,2′R,4S,4′S,5R,5′R,6R,6′R)-2,2′-(((((((((S)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Example 14).

### Example 15

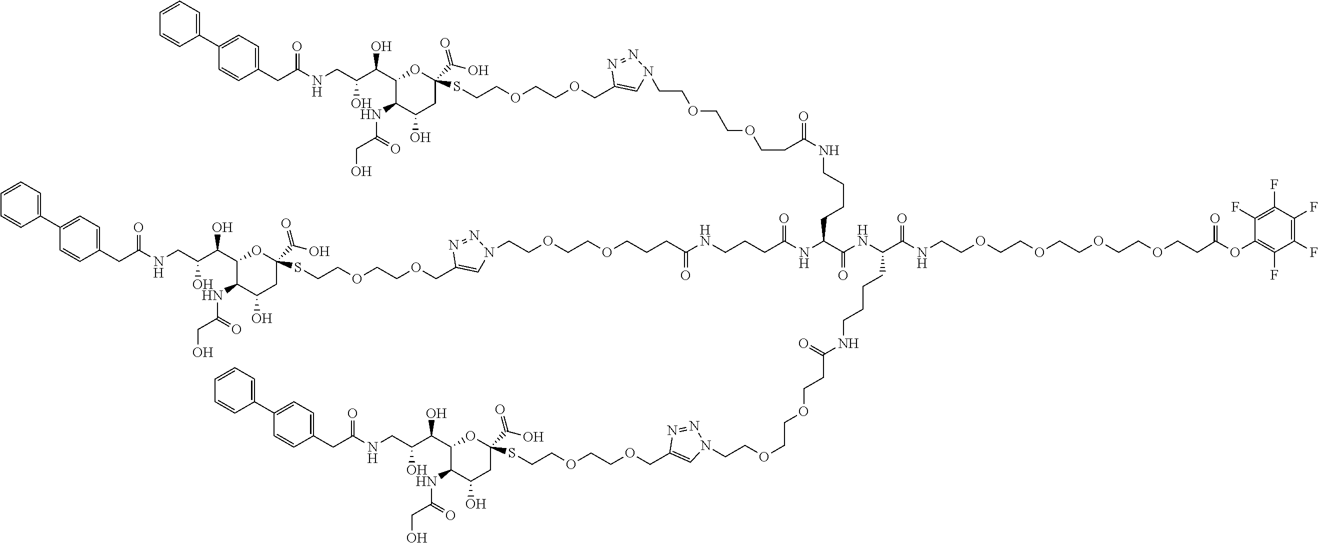 [SMILES: O=C(CCCOCCOCCn1cc(COCCOCCS[C@@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCCC(=O)N[C@@H](CCCCNC(=O)CCOCCOCCn1cc(COCCOCCS[C@@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)N[C@@H](CCCCNC(=O)CCOCCOCCn1cc(COCCOCCS[C@@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)NCCOCCOCCOCCOCCC(=O)Oc1c(F)c(F)c(F)c(F)c1F]

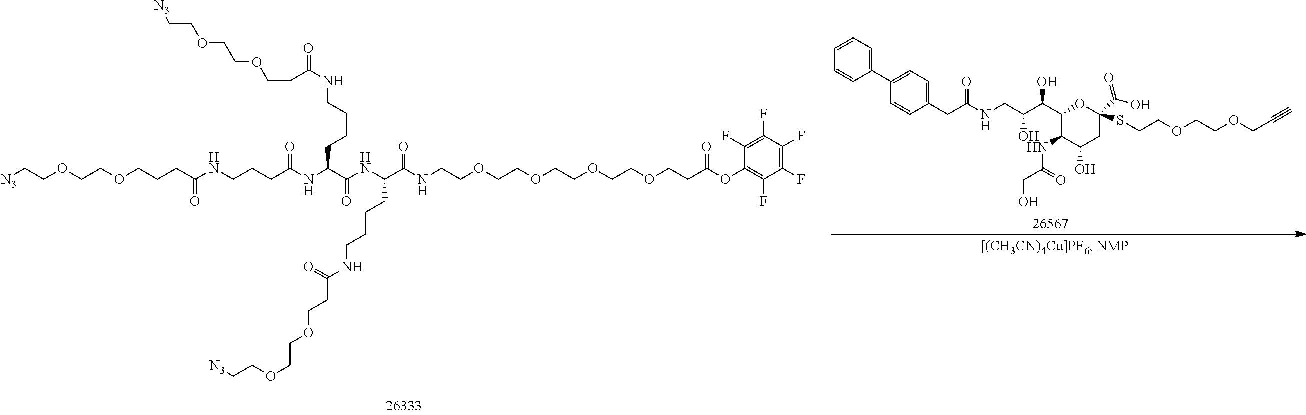 [SMILES: [N-]=[N+]=NCCOCCOCCCC(=O)NCCCC(=O)N[C@@H](CCCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F ; C#CCOCCOCCS[C@@]1(C(=O)O)C[C@H](O)[C@@H](NC(=O)CO)[C@H]([C@H](O)[C@H](O)CNC(=O)Cc2ccc(-c3ccccc3)cc2)O1]

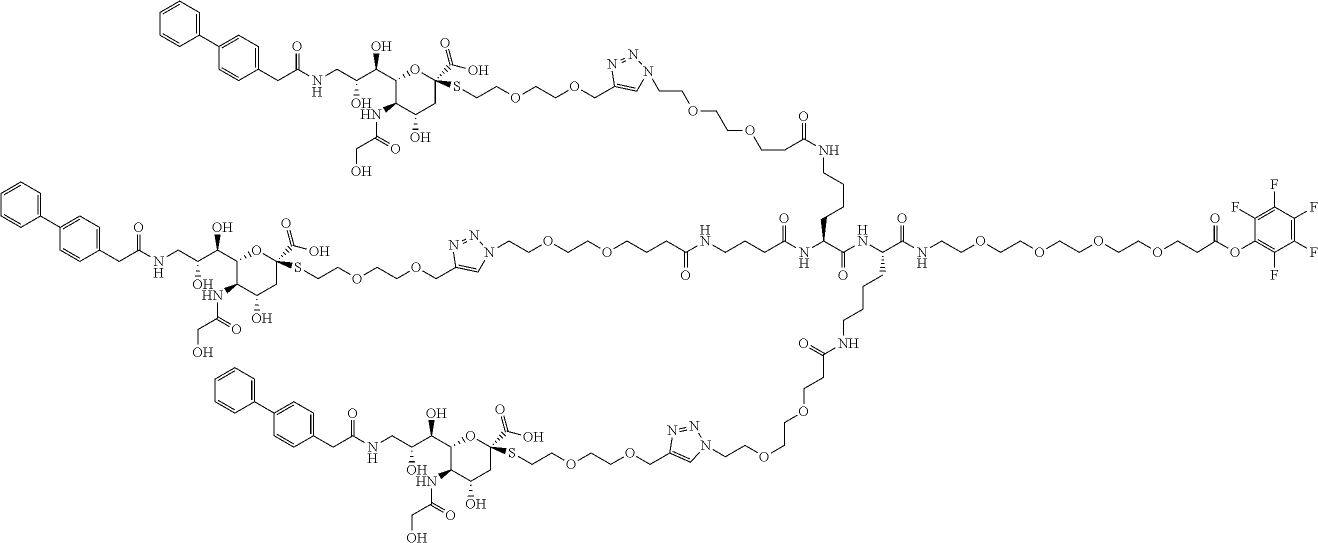 [SMILES: O=C(CCCOCCOCCn1cc(COCCOCCS[C@@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCCC(=O)N[C@@H](CCCCNC(=O)CCOCCOCCn1cc(COCCOCCS[C@@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)N[C@@H](CCCCNC(=O)CCOCCOCCn1cc(COCCOCCS[C@@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)NCCOCCOCCOCCOCCC(=O)Oc1c(F)c(F)c(F)c(F)c1F]

### Synthesis of Example 15

To a stirred solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (26567, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TEA). Fractions containing the desired product are combined and lyophilized to dryness to afford Example 15.

### (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)tetrahydro-2H-pyran-2-carboxylic acid (Example 16)

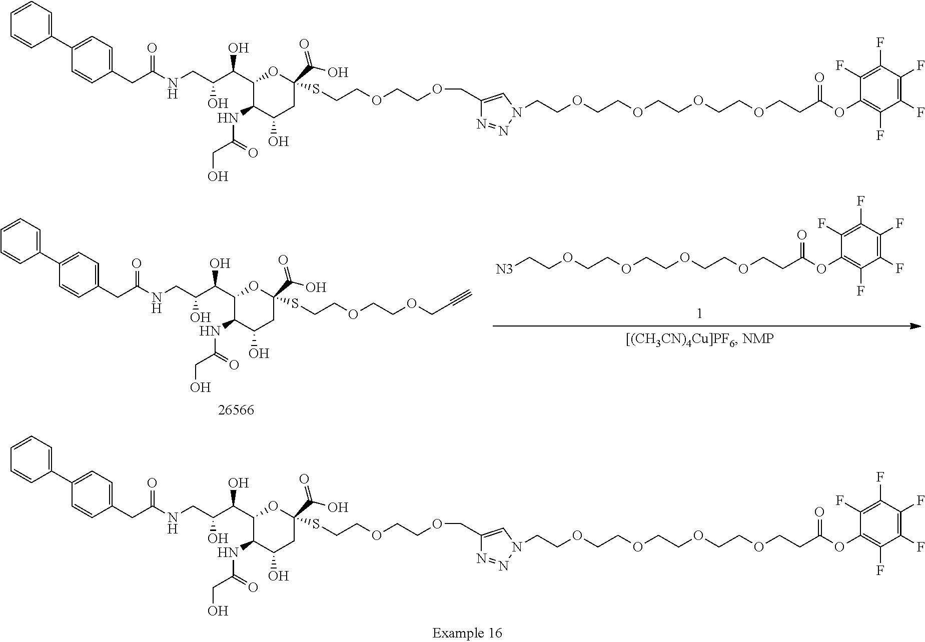 [SMILES: O=C(Cc1ccc(-c2ccccc2)cc1)NC[C@@H](O)[C@@H](O)[C@@H]1O[C@@](SCCOCCOCc2cn(CCOCCOCCOCCOCCC(=O)Oc3c(F)c(F)c(F)c(F)c3F)nn2)(C(=O)O)C[C@H](O)[C@H]1NC(=O)CO ; C#CCOCCOCCS[C@]1(C(=O)O)C[C@H](O)[C@@H](NC(=O)CO)[C@H]([C@H](O)[C@H](O)CNC(=O)Cc2ccc(-c3ccccc3)cc2)O1 ; [N-]=[N+]=NCCOCCOCCOCCOCCC(=O)Oc1c(F)c(F)c(F)c(F)c1F ; O=C(Cc1ccc(-c2ccccc2)cc1)NC[C@@H](O)[C@@H](O)[C@@H]1O[C@@](SCCOCCOCc2cn(CCOCCOCCOCCOCCC(=O)Oc3c(F)c(F)c(F)c(F)c3F)nn2)(C(=O)O)C[C@H](O)[C@H]1NC(=O)CO]

### Synthesis of (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)tetrahydro-2H-pyran-2-carboxylic acid (Example 16)

To a stirred solution of (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (26566, 1.00 eq) in NMP (0.3 mL) in a 1 dram vial is added a solution of perfluorophenyl 1-azido-3,6,9,12-tetraoxapentadecan-15-oate (1, 1.10 eq) in NMP (0.3 mL) followed by tetrakis(acetonitrile)copper(I) hexafluorophosphate (2.50 eq). The resulting solution is capped and stirred at room temperature for 10 min. The reaction is diluted with acetic acid, filtered, and purified via preparatory HPLC (15-65% acetonitrile in water with 0.1% TFA). Fractions containing the desired product are combined and lyophilized to dryness to afford (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-((2-(2-((1-(15-oxo-15-(perfluorophenoxy)-3,6,9,12-tetraoxapentadecyl)-1H-1,2,3-triazol-4-yl)methoxy)ethoxy)ethyl)thio)tetrahydro-2H-pyran-2-carboxylic acid (Example 16).

### (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(6-((1S,2S)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Example 17)

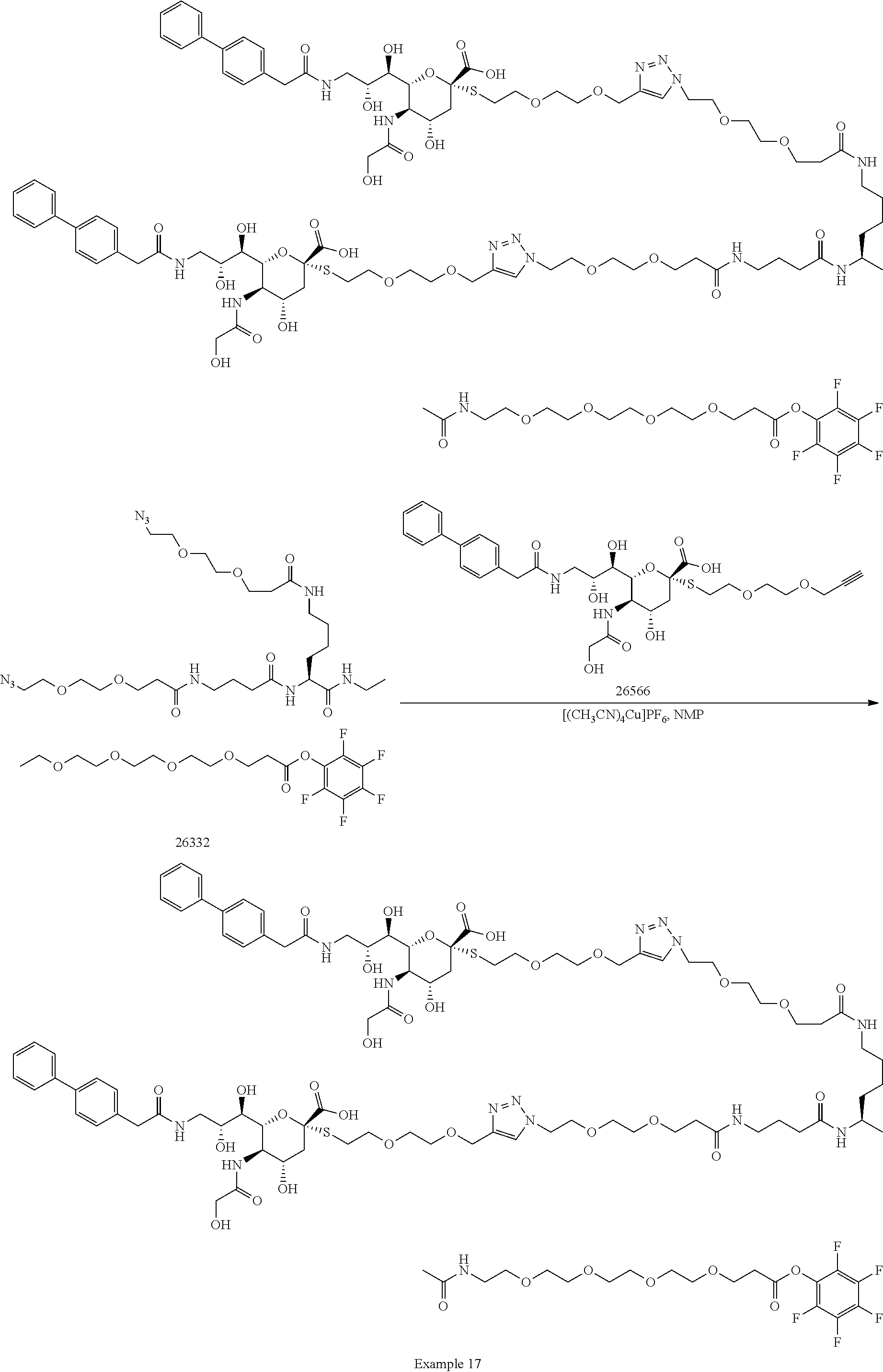

### Synthesis of (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(6-((1S,2S)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Example 17)

To a stirred solution of perfluorophenyl (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oate (26332, 1.00 eq) and (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (26566, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,2′R,4R,4′R,5S,5′S,6S,6′S)-2,2′-(((((((((R)-9,14,22-trioxo-16-((15-oxo-15-(perfluorophenoxy)-3,6,9,12-tetraoxapentadecyl)carbamoyl)-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(sulfanediyl))bis(6-((1S,2S)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Example 17).

### Example 18

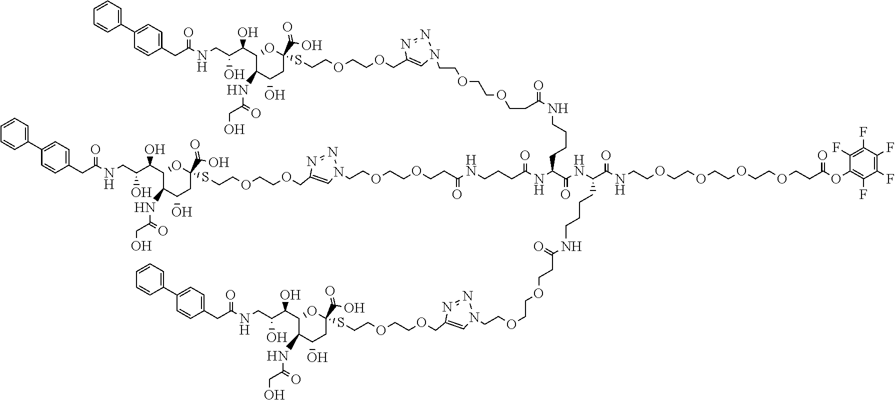 [SMILES: O=C(CCOCCOCCn1cc(COCCOCCS[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCCC[C@H](NC(=O)CCCNC(=O)CCOCCOCCn1cc(COCCOCCS[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)N[C@@H](CCCCNC(=O)CCOCCOCCn1cc(COCCOCCS[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)NCCOCCOCCOCCOCCC(=O)Oc1c(F)c(F)c(F)c(F)c1F]

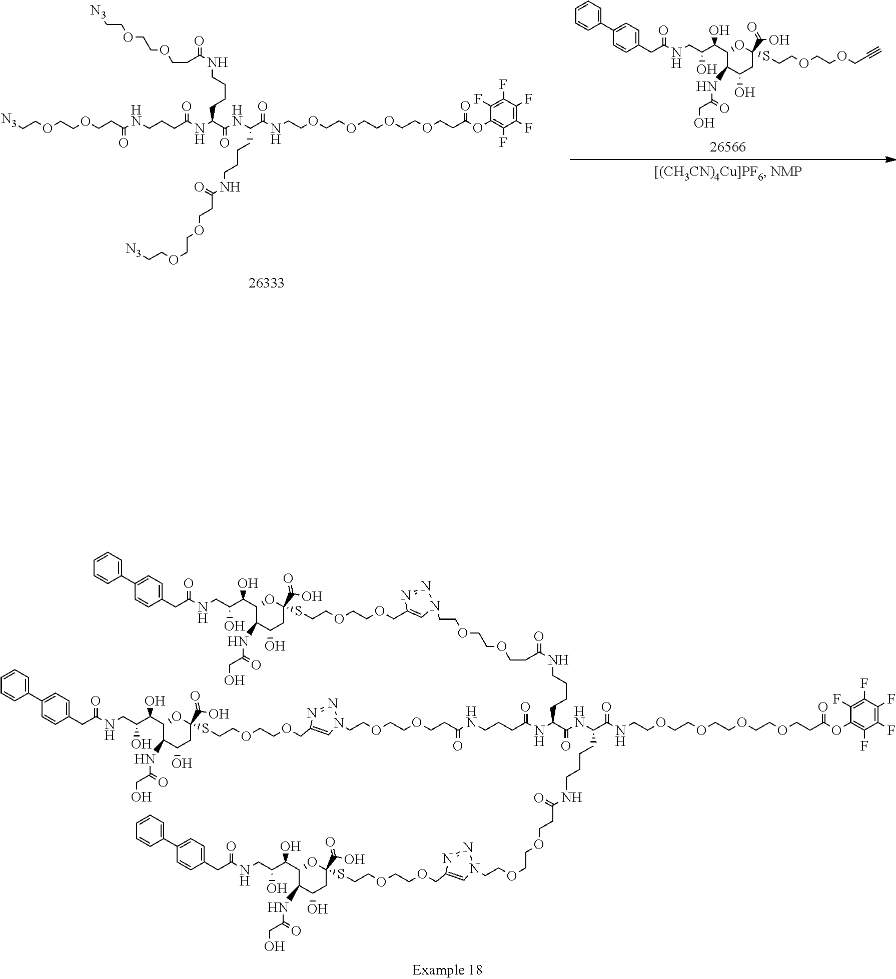

### Synthesis of Example 18

To a stirred solution of perfluorophenyl (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oate (26333, 1.00 eq) and (2R,4R,5S,6S)-6-((1S,2S)-3-(2-([1,1′-biphenyl]-4-yl)acetamido)-1,2-dihydroxypropyl)-4-hydroxy-5-(2-hydroxyacetamido)-2-((2-(2-(prop-2-yn-1-yloxy)ethoxy)ethyl)thio)tetrahydro-2H-pyran-2-carboxylic acid (26566, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford Example 18.

### (2R,4S,5R,6R)-6-((1R,2R)-3-(2-([1,1′-biphenyl]-4-yl)acetamido)-1,2-dihydroxypropyl)-2-(2-(2-((1-(4-(((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-(((S)-5-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-carboxypentyl)amino)-1-oxohexan-2-yl)amino)-4-oxobutyl)-1H-1,2,3-triazol-4-yl)methoxy)ethoxy)ethoxy)-4-hydroxy-5-(2-hydroxyacetamido)tetrahydro-2H-pyran-2-carboxylic acid (Example 19)

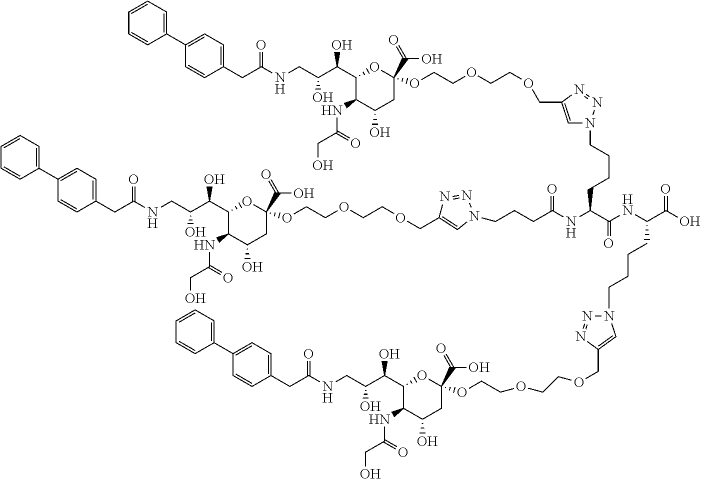 [SMILES: O=C(Cc1ccc(-c2ccccc2)cc1)NC[C@@H](O)[C@@H](O)[C@@H]1O[C@@](OCCOCCOCc2cn(CCCC[C@H](NC(=O)[C@H](CCCCn3cc(COCCOCCO[C@]4(C(=O)O)C[C@H](O)[C@@H](NC(=O)CO)[C@H]([C@H](O)[C@H](O)CNC(=O)Cc5ccc(-c6ccccc6)cc5)O4)nn3)NC(=O)CCCn3cc(COCCOCCO[C@]4(C(=O)O)C[C@H](O)[C@@H](NC(=O)CO)[C@H]([C@H](O)[C@H](O)CNC(=O)Cc5ccc(-c6ccccc6)cc5)O4)nn3)C(=O)O)nn2)(C(=O)O)C[C@H](O)[C@H]1NC(=O)CO]

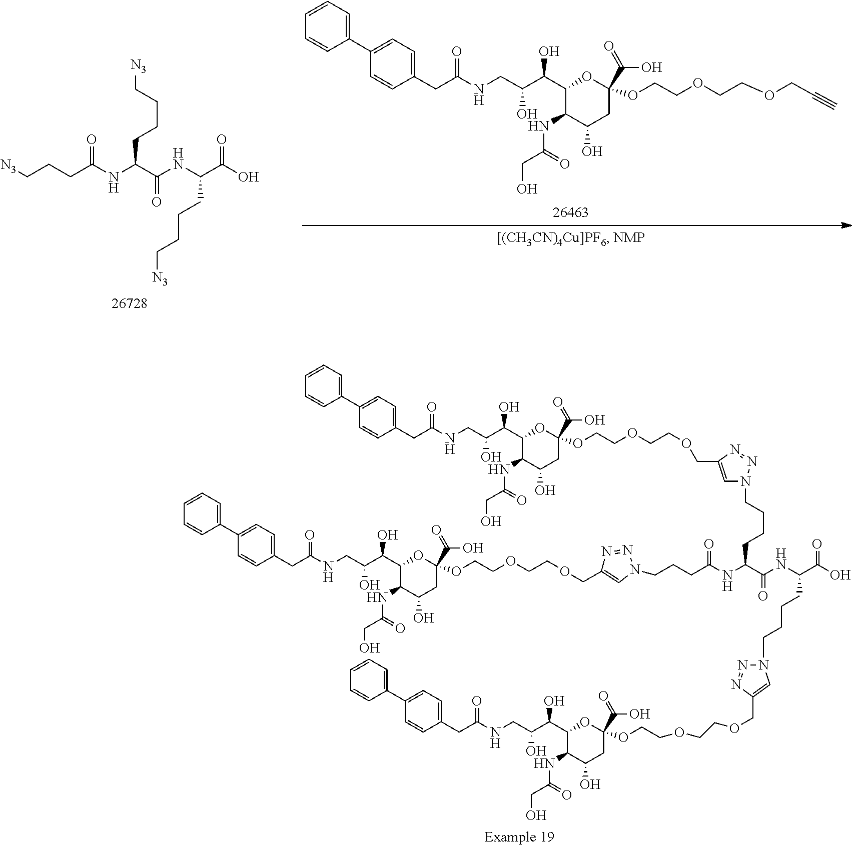

### Synthesis of (2R,4S,5R,6R)-6-((1R,2R)-3-(2-([1,1′-biphenyl]-4-yl)acetamido)-1,2-dihydroxypropyl)-2-(2-(2-((1-(4-(((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-(((S)-5-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-carboxypentyl)amino)-1-oxohexan-2-yl)amino)-4-oxobutyl)-1H-1,2,3-triazol-4-yl)methoxy)ethoxy)ethoxy)-4-hydroxy-5-(2-hydroxyacetamido)tetrahydro-2H-pyran-2-carboxylic acid (Example 19)

To a stirred solution of N2-(N2-(4-azidobutanoyl)-N6-diazo-D-lysyl)-N6-diazo-D-lysine (26728, 1.00 eq) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-6-((1R,2R)-3-(2-([1,1′-biphenyl]-4-yl)acetamido)-1,2-dihydroxypropyl)-2-(2-(2-((1-(4-(((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-(((S)-5-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-carboxypentyl)amino)-1-oxohexan-2-yl)amino)-4-oxobutyl)-1H-1,2,3-triazol-4-yl)methoxy)ethoxy)ethoxy)-4-hydroxy-5-(2-hydroxyacetamido)tetrahydro-2H-pyran-2-carboxylic acid (Example 19).

### (2R,4S,5R,6R)-5-acetamido-2-(2-(2-((1-(4-(((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-(((S)-5-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-carboxypentyl)amino)-1-oxohexan-2-yl)amino)-4-oxobutyl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 20)

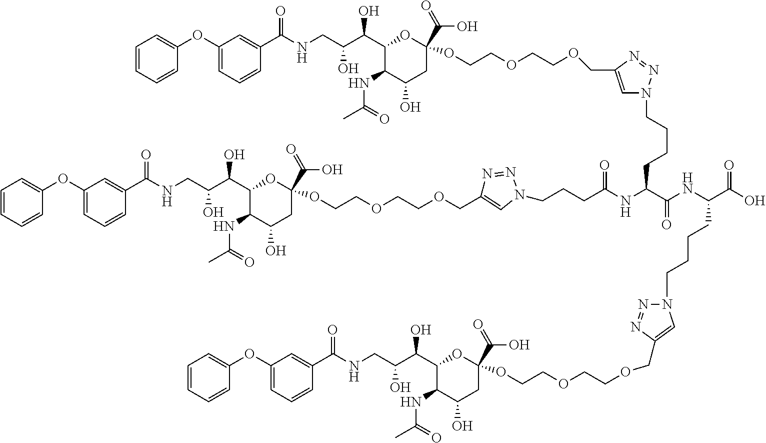 [SMILES: CC(=O)N[C@H]1[C@H]([C@H](O)[C@H](O)CNC(=O)c2cccc(Oc3ccccc3)c2)O[C@@](OCCOCCOCc2cn(CCCC[C@H](NC(=O)[C@H](CCCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)NC(=O)CCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)O)nn2)(C(=O)O)C[C@@H]1O]

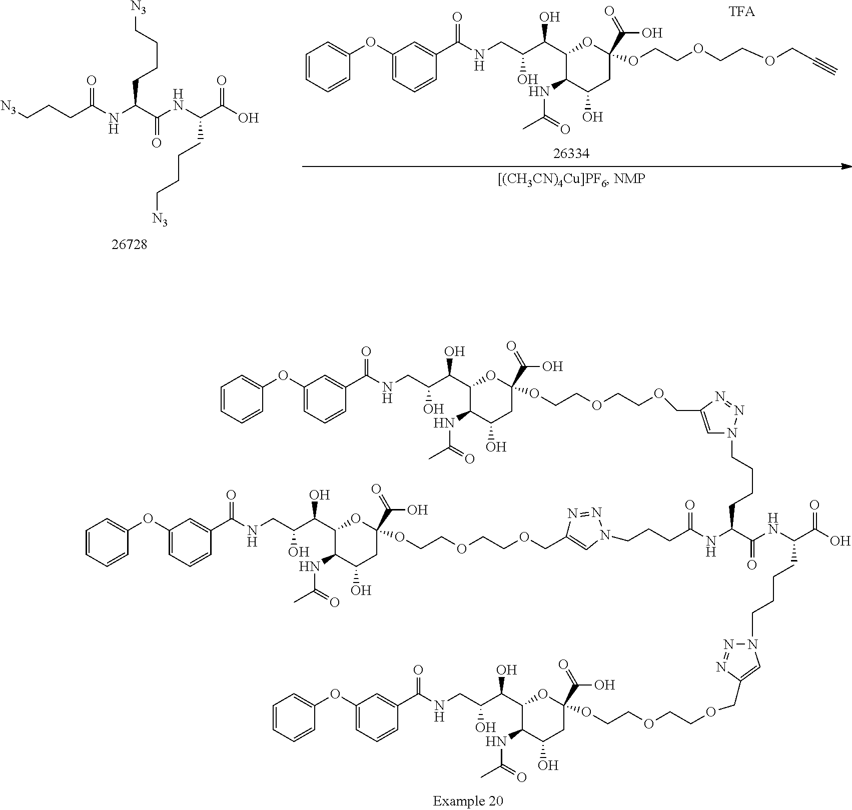

### Synthesis of (2R,4S,5R,6R)-5-acetamido-2-(2-(2-((1-(4-(((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-(((S)-5-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-carboxypentyl)amino)-1-oxohexan-2-yl)amino)-4-oxobutyl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 20)

To a stirred solution of N2-(N2-(4-azidobutanoyl)-N6-diazo-D-lysyl)-N6-diazo-D-lysine (26728, 1.00 eq) and (2R,4R,5S,6S)-4-hydroxy-5-(2-hydroxyacetamido)-2-((2-(2-(prop-2-yn-1-yloxy)ethoxy)ethyl)thio)-6-((1S,2S)-1,2,3-trihydroxypropyl)tetrahydro-2H-pyran-2-carboxylic acid (26334, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-5-acetamido-2-(2-(2-((1-(4-(((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-(((S)-5-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-carboxypentyl)amino)-1-oxohexan-2-yl)amino)-4-oxobutyl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 20).

### (2R,4S,5R,6R)-6-((1R,2R)-3-(2-([1,1′-biphenyl]-4-yl)acetamido)-1,2-dihydroxypropyl)-2-(2-(2-((1-(4-(((S)-5-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-carboxypentyl)amino)-4-oxobutyl)-1H-1,2,3-triazol-4-yl)methoxy)ethoxy)ethoxy)-4-hydroxy-5-(2-hydroxyacetamido)tetrahydro-2H-pyran-2-carboxylic acid (Example 21)

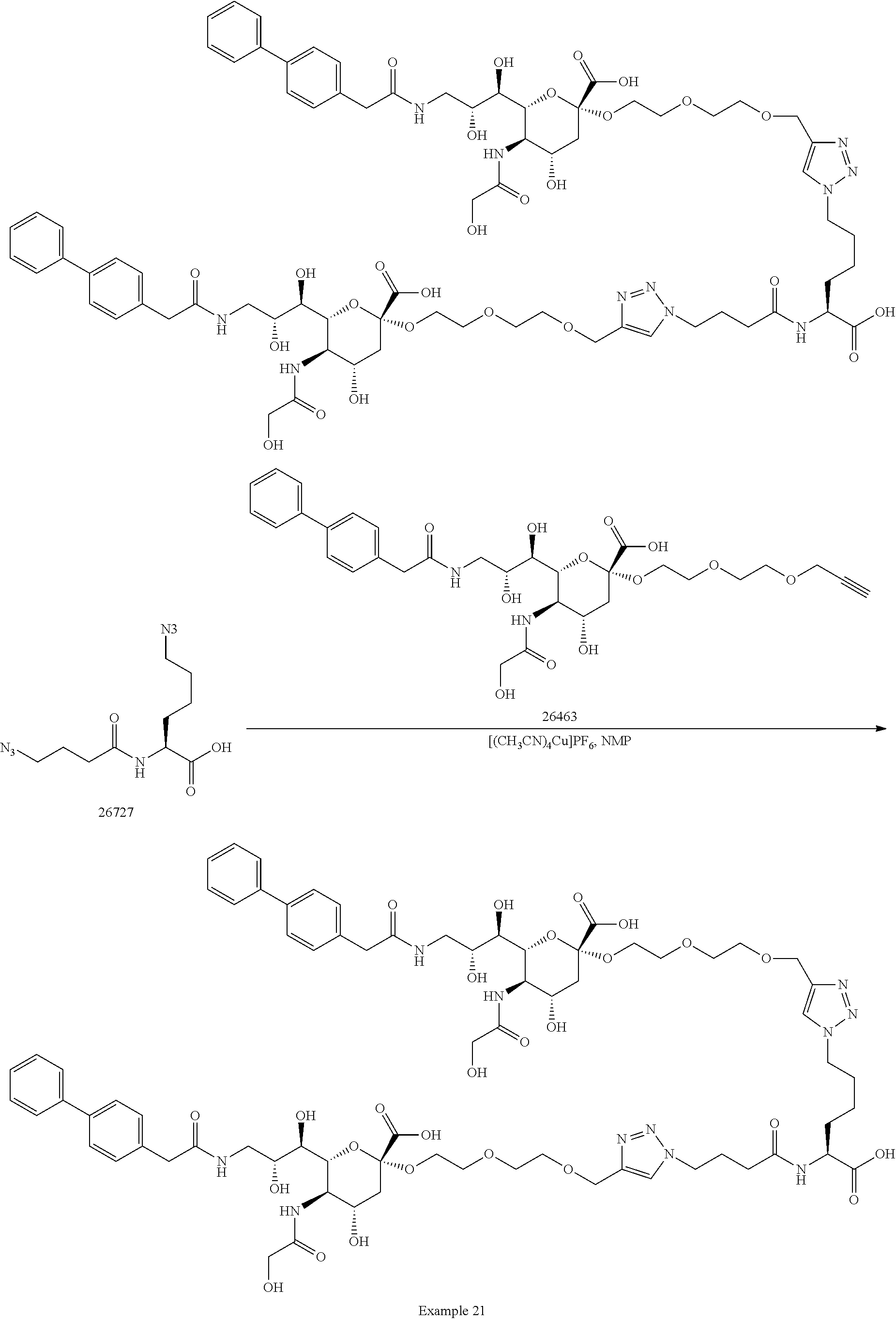

### Synthesis of (2R,4S,5R,6R)-6-((1R,2R)-3-(2-([1,1′-biphenyl]-4-yl)acetamido)-1,2-dihydroxypropyl)-2-(2-(2-((1-(4-(((S)-5-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-carboxypentyl)amino)-4-oxobutyl)-1H-1,2,3-triazol-4-yl)methoxy)ethoxy)ethoxy)-4-hydroxy-5-(2-hydroxyacetamido)tetrahydro-2H-pyran-2-carboxylic acid (Example 21)

To a stirred solution of N2-(4-azidobutanoyl)-N6-diazo-D-lysine (26727, 1.00 eq) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-6-((1R,2R)-3-(2-([1,1′-biphenyl]-4-yl)acetamido)-1,2-dihydroxypropyl)-2-(2-(2-((1-(4-(((S)-5-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-carboxypentyl)amino)-4-oxobutyl)-1H-1,2,3-triazol-4-yl)methoxy)ethoxy)ethoxy)-4-hydroxy-5-(2-hydroxyacetamido)tetrahydro-2H-pyran-2-carboxylic acid (Example 21).

### (2R,4S,5R,6R)-5-acetamido-2-(2-(2-((1-(4-(((S)-5-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-carboxypentyl)amino)-4-oxobutyl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 22)

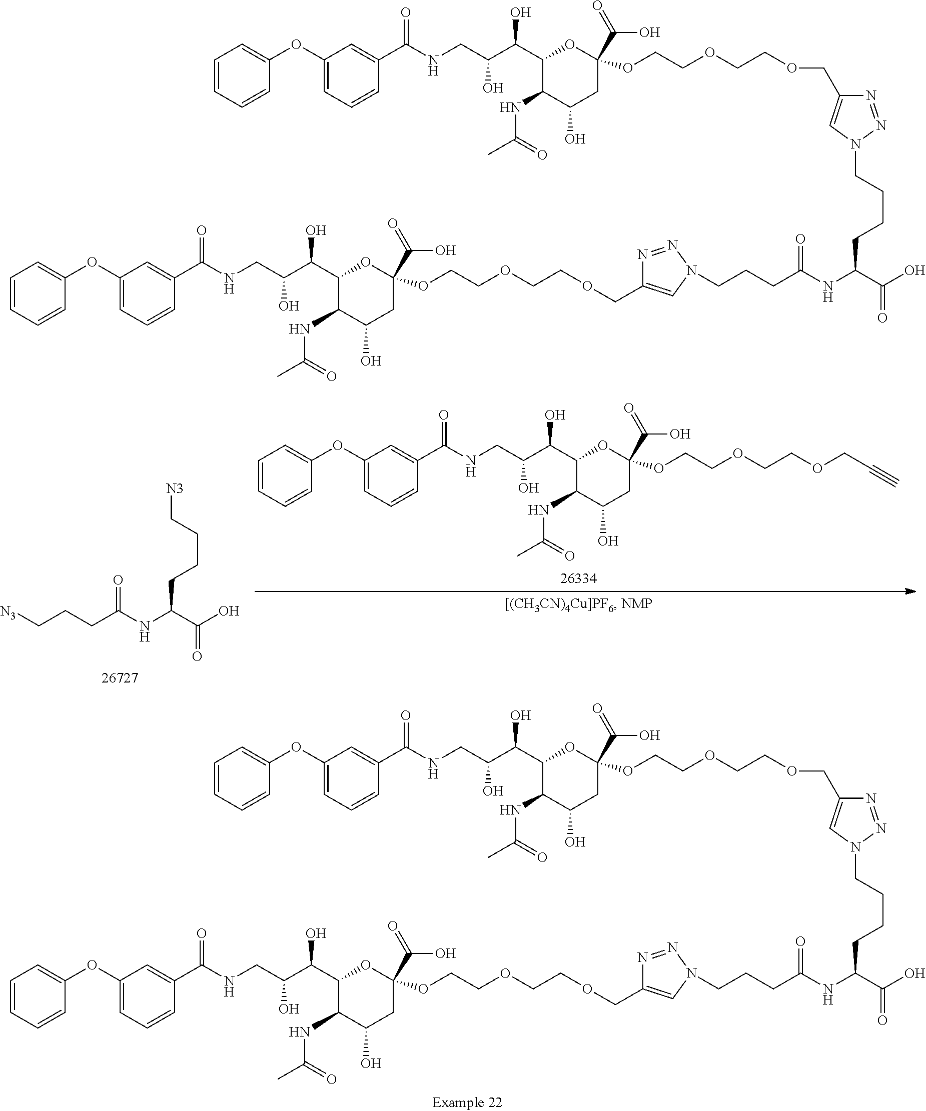

### Synthesis of (2R,4S,5R,6R)-5-acetamido-2-(2-(2-((1-(4-(((S)-5-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-carboxypentyl)amino)-4-oxobutyl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 22)

To a stirred solution of N2-(4-azidobutanoyl)-N6-diazo-D-lysine (26727, 1.00 eq) and (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-5-acetamido-2-(2-(2-((1-(4-(((S)-5-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-carboxypentyl)amino)-4-oxobutyl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 22).

### (2R,4S,5R,6R)-6-((1R,2R)-3-(2-([1,1′-biphenyl]-4-yl)acetamido)-1,2-dihydroxypropyl)-2-(2-(2-((1-(4-(((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-(((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-oxo-1-(perfluorophenoxy)hexan-2-yl)amino)-1-oxohexan-2-yl)amino)-4-oxobutyl)-1H-1,2,3-triazol-4-yl)methoxy)ethoxy)ethoxy)-4-hydroxy-5-(2-hydroxyacetamido)tetrahydro-2H-pyran-2-carboxylic acid (Example 23)

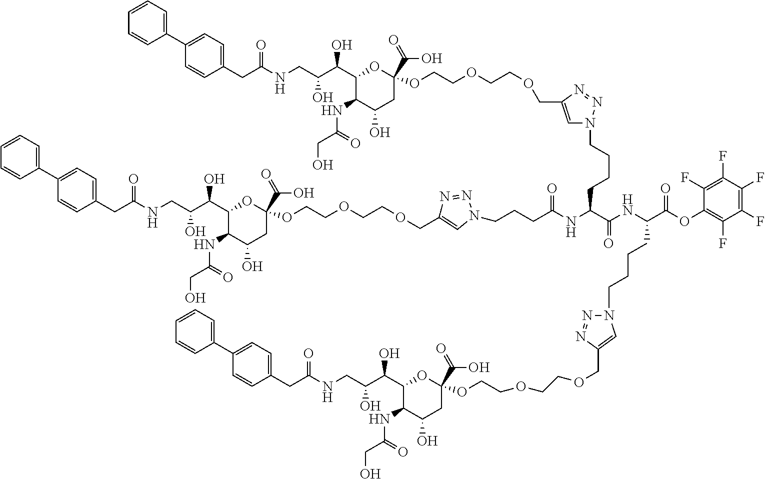 [SMILES: O=C(Cc1ccc(-c2ccccc2)cc1)NC[C@@H](O)[C@@H](O)[C@@H]1O[C@@](OCCOCCOCc2cn(CCCC[C@H](NC(=O)CCCn3cc(COCCOCCO[C@]4(C(=O)O)C[C@H](O)[C@@H](NC(=O)CO)[C@H]([C@H](O)[C@H](O)CNC(=O)Cc5ccc(-c6ccccc6)cc5)O4)nn3)C(=O)N[C@@H](CCCCn3cc(COCCOCCO[C@]4(C(=O)O)C[C@H](O)[C@@H](NC(=O)CO)[C@H]([C@H](O)[C@H](O)CNC(=O)Cc5ccc(-c6ccccc6)cc5)O4)nn3)C(=O)Oc3c(F)c(F)c(F)c(F)c3F)nn2)(C(=O)O)C[C@H](O)[C@H]1NC(=O)CO]

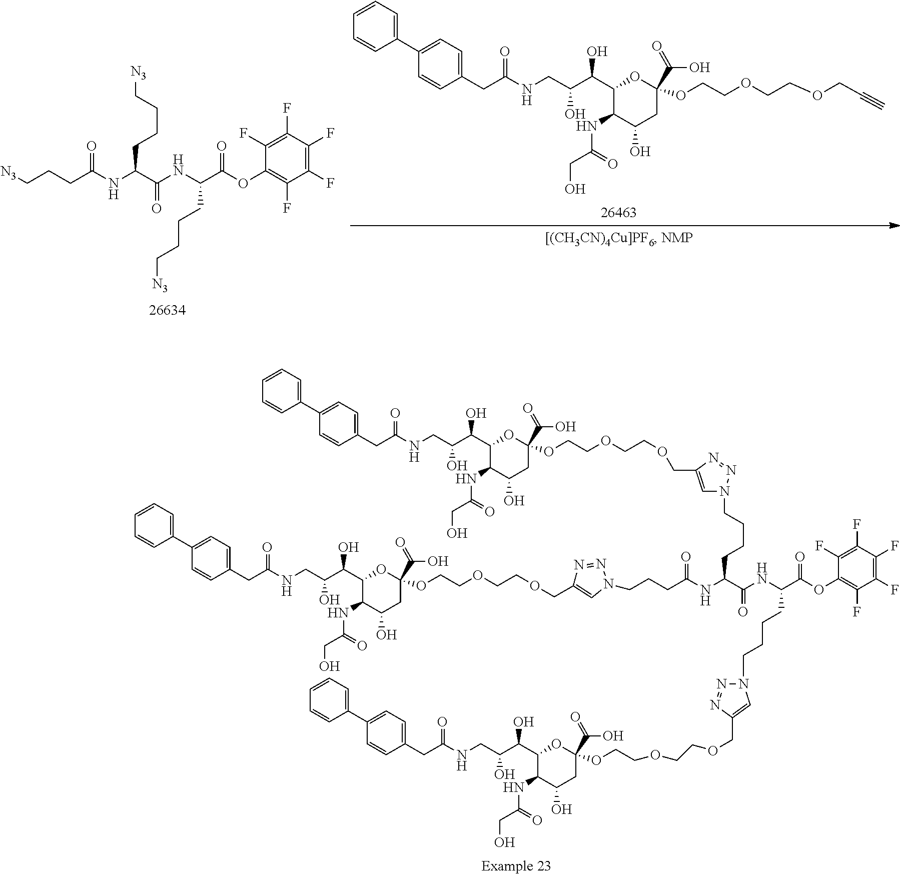

### Synthesis of (2R,4S,5R,6R)-6-((1R,2R)-3-(2-([1,1′-biphenyl]-4-yl)acetamido)-1,2-dihydroxypropyl)-2-(2-(2-((1-(4-(((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)eth oxy)ethoxy)methyl)-1H-1,2,3-triazol-1-yl)-1-(((S)-6-(4-((2-(2-(((2R,4S,5R,6R)-6-((1R,2R)-3-(2-([1,1′-biphenyl]-4-yl) acetamido)-1,2-dihydroxypropyl)-2-carboxy-4-hydroxy-5-(2-hydroxyacetamido)tetrahydro-2H-pyran-2-yl)oxy)eth oxy) ethoxy)meth yl)-1H-1,2,3-triazol-1-yl)-1-oxo-1-(perfluorophenoxy)hexan-2-yl)amino)-1-oxohexan-2-yl)amino)-4-oxobutyl)-1H-1, 2, 3-triazol-4-yl)methoxy)ethoxy)ethoxy)-4-hydroxy-5-(2-hydroxyacetamido)tetrahydro-2H-pyran-2-carboxylic acid (Example 23)

To a stirred solution of perfluorophenyl N2-(N2-(4-azidobutanoyl)-N6-diazo-D-lysyl)-N6-diazo-D-lysinate (26634, 1.00 eq) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-6-((1R,2R)-3-(2-([1,1′-biphenyl]-4-yl)acetamido)-1,2-dihydroxypropyl)-2-(2-(2-((1-(4-(((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-(((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-1-oxo-1-(perfluorophenoxy)hexan-2-yl)amino)-1-oxohexan-2-yl)amino)-4-oxobutyl)-1H-1,2,3-triazol-4-yl)methoxy)ethoxy)ethoxy)-4-hydroxy-5-(2-hydroxyacetamido)tetrahydro-2H-pyran-2-carboxylic acid (Example 23).

### (2R,4S,5R,6R)-5-acetamido-2-(2-(2-((1-(4-(((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-(((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-oxo-1-(perfluorophenoxy)hexan-2-yl)amino)-1-oxohexan-2-yl)amino)-4-oxobutyl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 24)

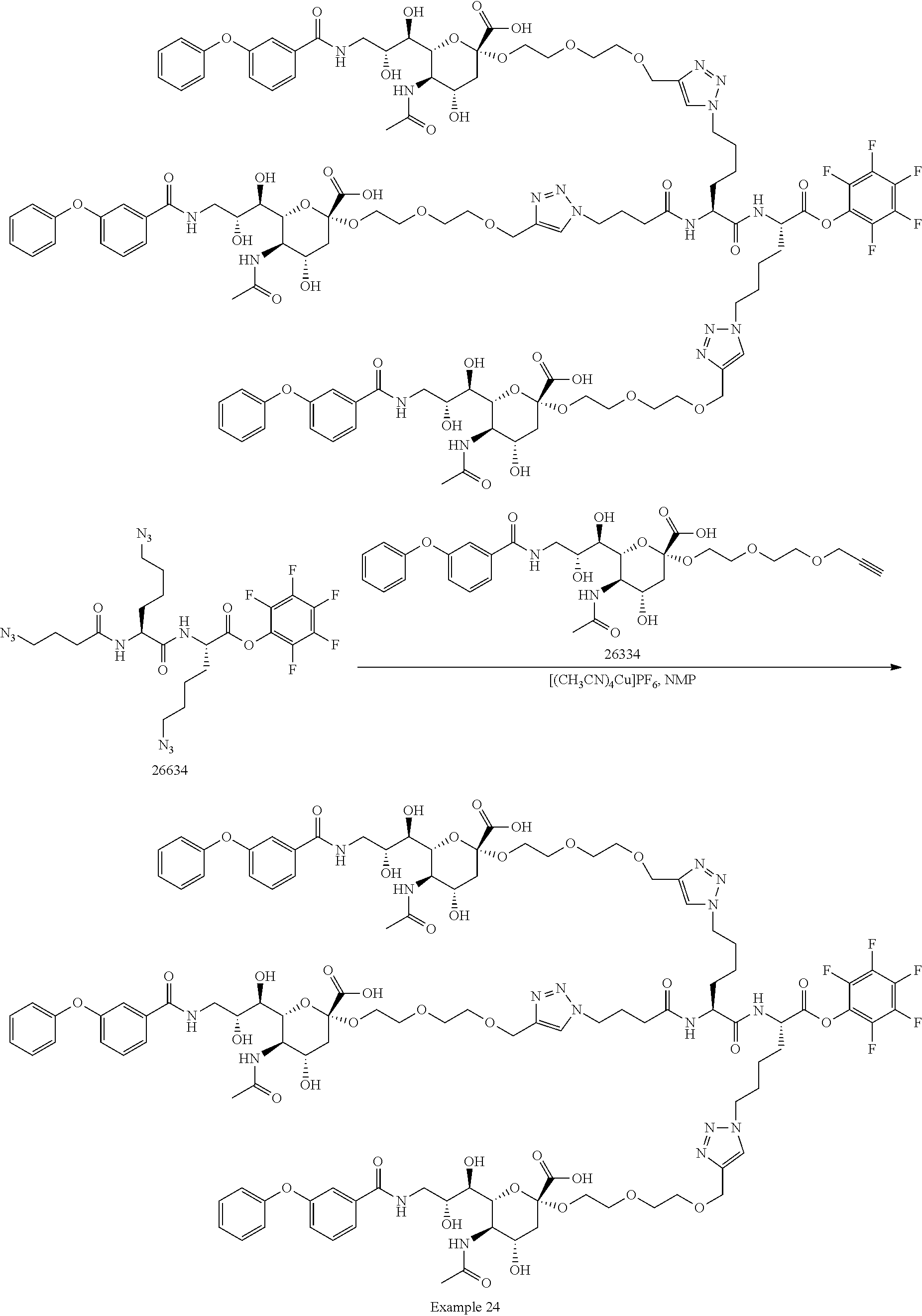

### Synthesis of (2R,4S,5R,6R)-5-acetamido-2-(2-(2-((1-(4-(((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)eth oxy)ethoxy)methyl)-1H-1,2,3-triazol-1-yl)-1-(((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-oxo-1-(perfluorophenoxy)hexan-2-yl)amino)-1-oxohexan-2-yl)amino)-4-oxobutyl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 24)

To a stirred solution of perfluorophenyl N2-(N2-(4-azidobutanoyl)-N6-diazo-D-lysyl)-N6-diazo-D-lysinate (26634, 1.00 eq) and (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-5-acetamido-2-(2-(2-((1-(4-(((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-(((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-1-oxo-1-(perfluorophenoxy)hexan-2-yl)amino)-1-oxohexan-2-yl)amino)-4-oxobutyl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 24).

### (2R,4S,5R,6R)-6-((1R,2R)-3-(2-([1,1′-biphenyl]-4-yl)acetamido)-1,2-dihydroxypropyl)-2-(2-(2-((1-((S)-16-((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-2-(4-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)butanamido)hexanamido)-1-oxo-1-(perfluorophenoxy)-4,7,10,13-tetraoxaicosan-20-yl)-1H-1,2,3-triazol-4-yl)methoxy)ethoxy)ethoxy)-4-hydroxy-5-(2-hydroxyacetamido)tetrahydro-2H-pyran-2-carboxylic acid (Example 25)

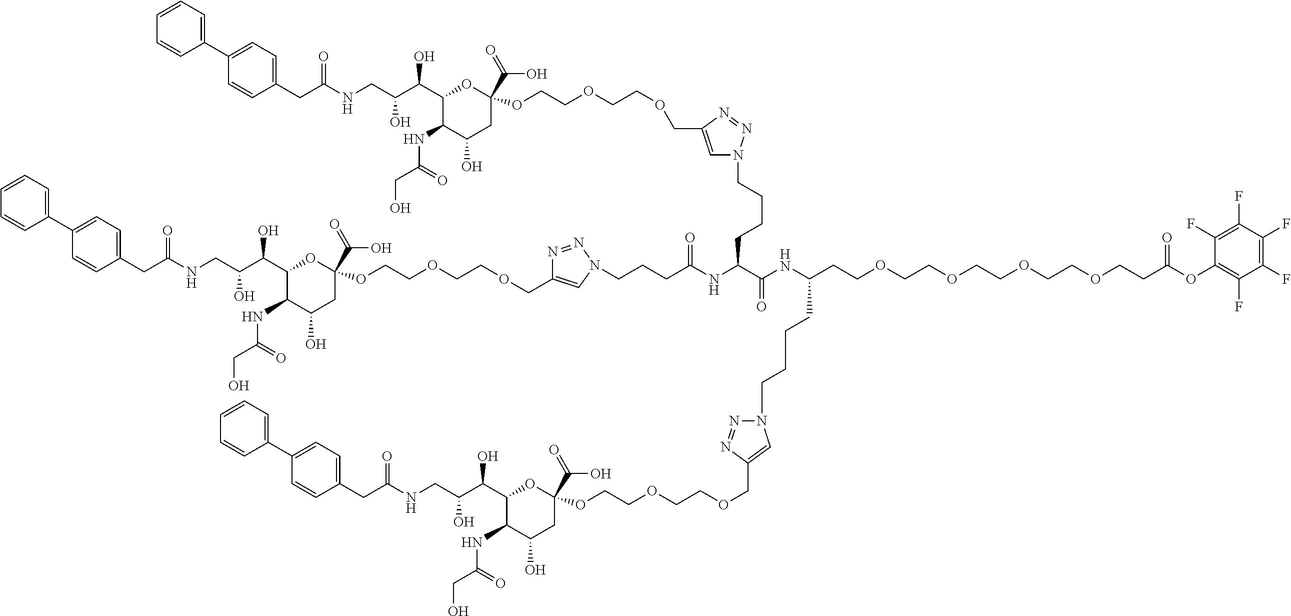 [SMILES: O=C(Cc1ccc(-c2ccccc2)cc1)NC[C@@H](O)[C@@H](O)[C@@H]1O[C@@](OCCOCCOCc2cn(CCCC[C@@H](CCOCCOCCOCCOCCC(=O)Oc3c(F)c(F)c(F)c(F)c3F)NC(=O)[C@H](CCCCn3cc(COCCOCCO[C@]4(C(=O)O)C[C@H](O)[C@@H](NC(=O)CO)[C@H]([C@H](O)[C@H](O)CNC(=O)Cc5ccc(-c6ccccc6)cc5)O4)nn3)NC(=O)CCCn3cc(COCCOCCO[C@]4(C(=O)O)C[C@H](O)[C@@H](NC(=O)CO)[C@H]([C@H](O)[C@H](O)CNC(=O)Cc5ccc(-c6ccccc6)cc5)O4)nn3)nn2)(C(=O)O)C[C@H](O)[C@H]1NC(=O)CO]

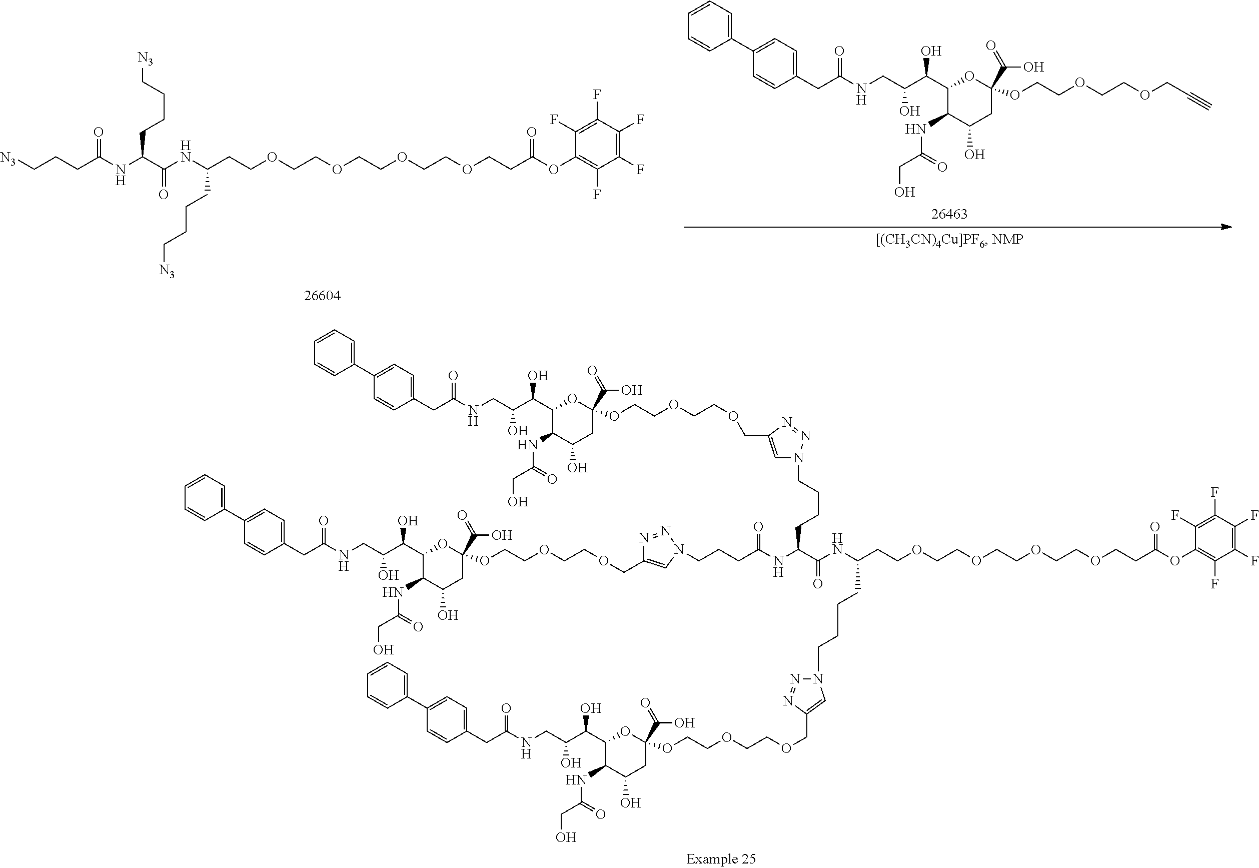

### Synthesis of (2R,4S,5R,6R)-6-((1R,2R)-3-(2-([1,1′-biphenyl]-4-yl)acetamido)-1,2-dihydroxypropyl)-2-(2-(2-((1-((S)-16-((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-2-(4-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)butanamido)hexanamido)-1-oxo-1-(perfluorophenoxy)-4,7,10,13-tetraoxaicosan-20-yl)-1H-1,2,3-triazol-4-yl)methoxy)ethoxy)ethoxy)-4-hydroxy-5-(2-hydroxyacetamido)tetrahydro-2H-pyran-2-carboxylic acid (Example 25)

To a stirred solution of perfluorophenyl (16R,19R)-24-azido-16,19-bis(4-azidobutyl)-18,21-dioxo-4,7,10,13-tetraoxa-17,20-diazatetracosanoate (26604, 1.00 eq) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-6-((1R,2R)-3-(2-([1,1′-biphenyl]-4-yl)acetamido)-1,2-dihydroxypropyl)-2-(2-(2-((1-((S)-16-((S)-6-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)-2-(4-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)butanamido)hexanamido)-1-oxo-1-(perfluorophenoxy)-4,7,10,13-tetraoxaicosan-20-yl)-1H-1,2,3-triazol-4-yl)methoxy)ethoxy)ethoxy)-4-hydroxy-5-(2-hydroxyacetamido)tetrahydro-2H-pyran-2-carboxylic acid (Example 25).

### (2R,4S,5R,6R)-5-acetamido-2-(2-(2-((1-((5)-16-((5)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-2-(4-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)butanamido)hexanamido)-1-oxo-1-(perfluorophenoxy)-4,7,10,13-tetraoxaicosan-20-yl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 26)

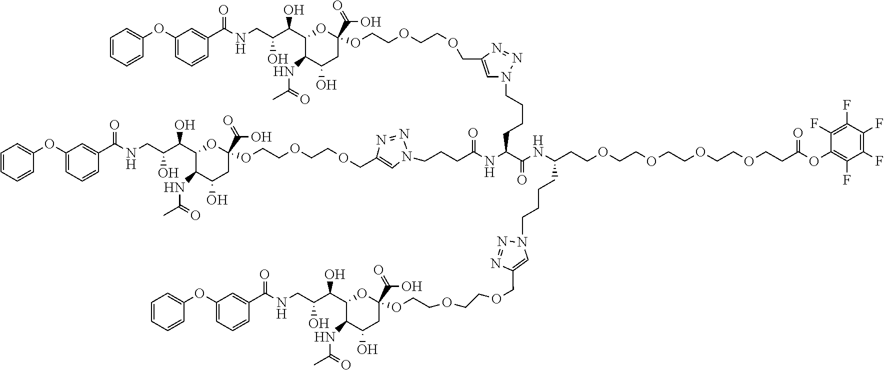 [SMILES: CC(=O)N[C@H]1[C@H]([C@H](O)[C@H](O)CNC(=O)c2cccc(Oc3ccccc3)c2)O[C@@](OCCOCCOCc2cn(CCCC[C@@H](CCOCCOCCOCCOCCC(=O)Oc3c(F)c(F)c(F)c(F)c3F)NC(=O)[C@H](CCCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)NC(=O)CCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)nn2)(C(=O)O)C[C@@H]1O]

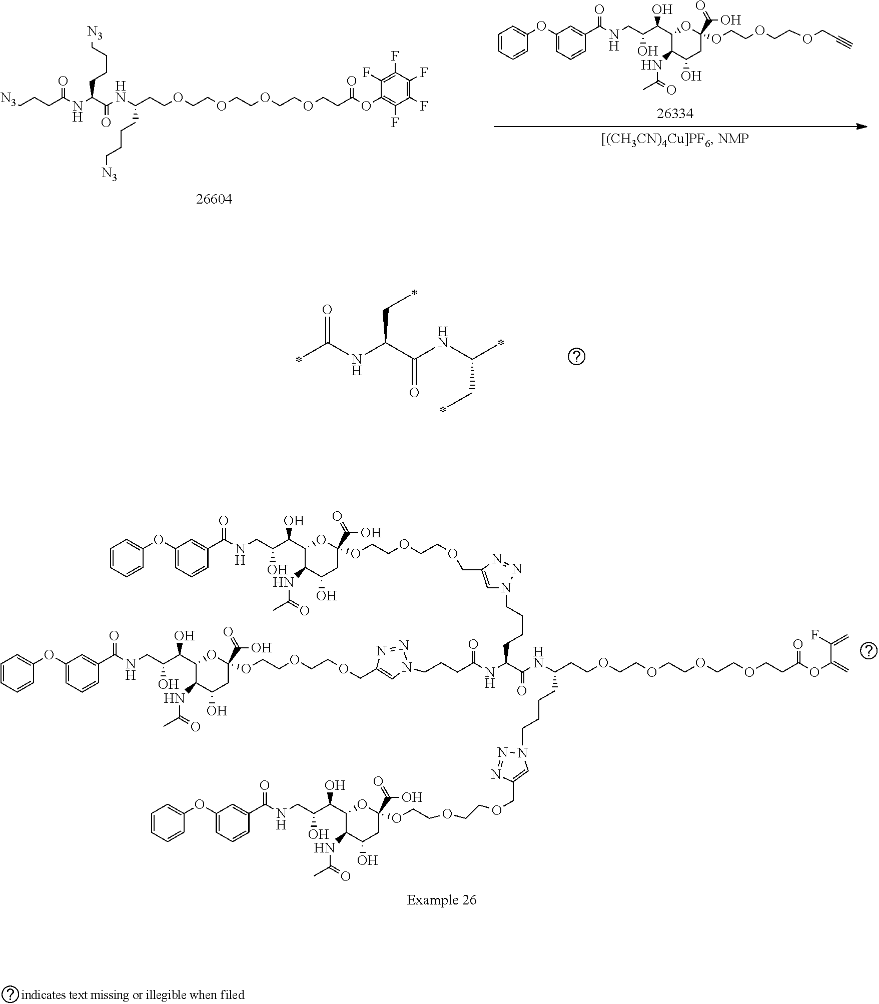

### Synthesis of (2R,4S,5R,6R)-5-acetamido-2-(2-(2-((1-((S)-16-((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-2-(4-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)butanamido)hexanamido)-1-oxo-1-(perfluorophenoxy)-4,7,10,13-tetraoxaicosan-20-yl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 26)

To a stirred solution of perfluorophenyl (16R,19R)-24-azido-16,19-bis(4-azidobutyl)-18,21-dioxo-4,7,10,13-tetraoxa-17,20-diazatetracosanoate (26604, 1.00 eq) and (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-5-acetamido-2-(2-(2-((1-((S)-16-((S)-6-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)-2-(4-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)butanamido)hexanamido)-1-oxo-1-(perfluorophenoxy)-4,7,10,13-tetraoxaicosan-20-yl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 26).

### (2R,4S,5R,6R)-6-((1R,2R)-3-(2-([1,1′-biphenyl]-4-yl)acetamido)-1,2-dihydroxypropyl)-2-(2-(2-((1-((S)-18-(4-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)butanamido)-1,17-dioxo-1-(perfluorophenoxy)-4,7,10,13-tetraoxa-16-azadocosan-22-yl)-1H-1,2,3-triazol-4-yl)methoxy)ethoxy)ethoxy)-4-hydroxy-5-(2-hydroxyacetamido)tetrahydro-2H-pyran-2-carboxylic acid (Example 27)

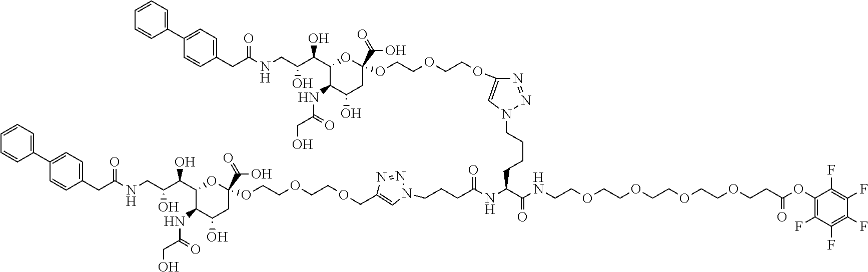 [SMILES: O=C(Cc1ccc(-c2ccccc2)cc1)NC[C@@H](O)[C@@H](O)[C@@H]1O[C@@](OCCOCCOCc2cn(CCCC(=O)N[C@@H](CCCCn3cc(OCCOCCO[C@]4(C(=O)O)C[C@H](O)[C@@H](NC(=O)CO)[C@H]([C@H](O)[C@H](O)CNC(=O)Cc5ccc(-c6ccccc6)cc5)O4)nn3)C(=O)NCCOCCOCCOCCOCCC(=O)Oc3c(F)c(F)c(F)c(F)c3F)nn2)(C(=O)O)C[C@H](O)[C@H]1NC(=O)CO]

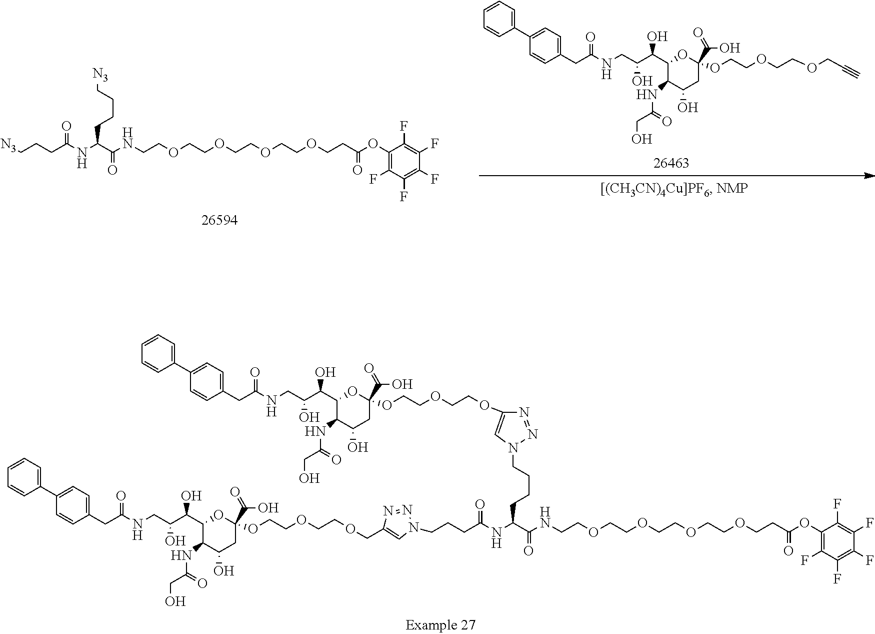 [SMILES: [N-]=[N+]=NCCCC[C@H](NC(=O)CCCN=[N+]=[N-])C(=O)NCCOCCOCCOCCOCCC(=O)Oc1c(F)c(F)c(F)c(F)c1F ; C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1 ; O=C(Cc1ccc(-c2ccccc2)cc1)NC[C@@H](O)[C@@H](O)[C@@H]1O[C@@](OCCOCCOCc2cn(CCCC(=O)N[C@@H](CCCCn3cc(OCCOCCO[C@]4(C(=O)O)C[C@H](O)[C@@H](NC(=O)CO)[C@H]([C@H](O)[C@H](O)CNC(=O)Cc5ccc(-c6ccccc6)cc5)O4)nn3)C(=O)NCCOCCOCCOCCOCCC(=O)Oc3c(F)c(F)c(F)c(F)c3F)nn2)(C(=O)O)C[C@H](O)[C@H]1NC(=O)CO]

### Synthesis of (2R,4S,5R,6R)-6-((1R,2R)-3-(2-([1,1′-biphenyl]-4-yl)acetamido)-1,2-dihydroxypropyl)-2-(2-(2-((1-((S)-18-(4-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)butanamido)-1,17-dioxo-1-(perfluorophenoxy)-4,7,10,13-tetraoxa-16-azadocosan-22-yl)-1H-1,2,3-triazol-4-yl)methoxy)ethoxy)ethoxy)-4-hydroxy-5-(2-hydroxyacetamido)tetrahydro-2H-pyran-2-carboxylic acid (Example 27)

To a stirred solution of perfluorophenyl (R)-23-azido-18-(4-azidobutyl)-17,20-dioxo-4,7,10,13-tetraoxa-16,19-diazatricosanoate (26594, 1.00 eq) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 3.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-6-((1R,2R)-3-(2-([1,1′-biphenyl]-4-yl)acetamido)-1,2-dihydroxypropyl)-2-(2-(2-((1-((S)-18-(4-(4-((2-(2-(((2R,4S,5R,6R)-6-((1R,2R)-3-(2-([1,1′-biphenyl]-4-yl)acetamido)-1,2-dihydroxypropyl)-2-carboxy-4-hydroxy-5-(2-hydroxyacetamido)tetrahydro-2H-pyran-2-yl)oxy)ethoxy)ethoxy)methyl)-1H-1,2,3-triazol-1-yl)butanamido)-1,17-dioxo-1-(perfluorophenoxy)-4,7,10,13-tetraoxa-16-azadocosan-22-yl)-1H-1,2,3-triazol-4-yl)methoxy)ethoxy)ethoxy)-4-hydroxy-5-(2-hydroxyacetamido)tetrahydro-2H-pyran-2-carboxylic acid (Example 27).

### (2R,4S,5R,6R)-5-acetamido-2-(2-(2-((1-((S)-18-(4-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)butanamido)-1,17-dioxo-1-(perfluorophenoxy)-4,7,10,13-tetraoxa-16-azadocosan-22-yl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 28)

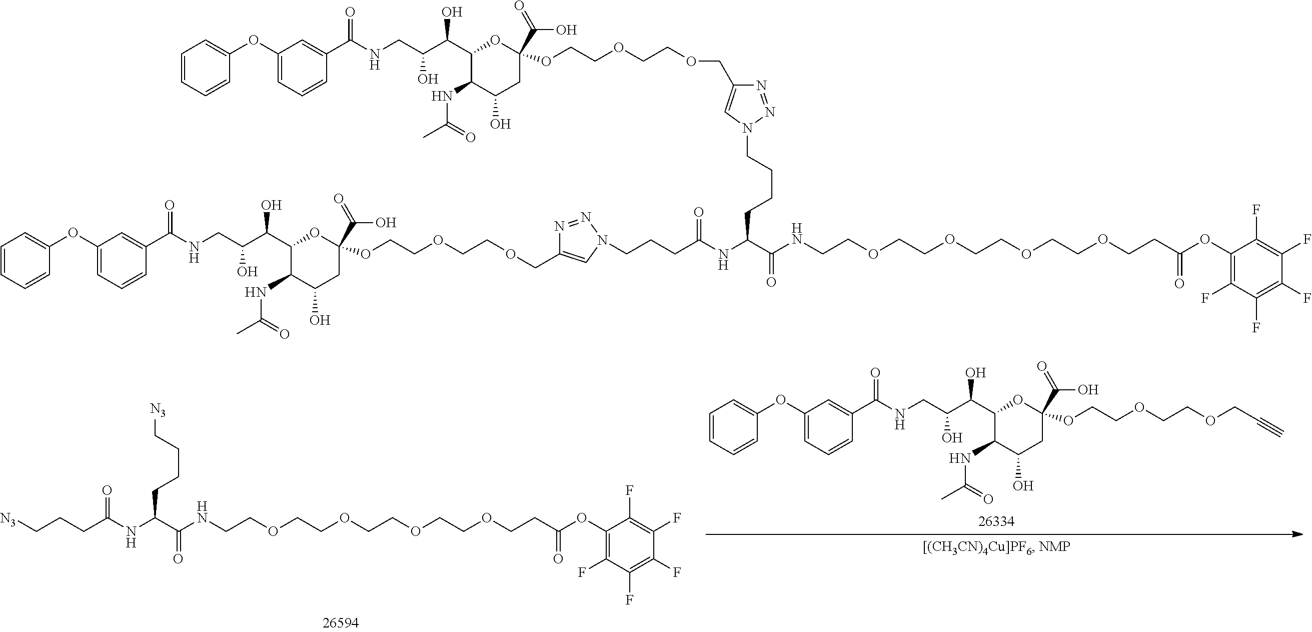 [SMILES: CC(=O)N[C@H]1[C@H]([C@H](O)[C@H](O)CNC(=O)c2cccc(Oc3ccccc3)c2)O[C@@](OCCOCCOCc2cn(CCCC[C@H](NC(=O)CCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)NCCOCCOCCOCCOCCC(=O)Oc3c(F)c(F)c(F)c(F)c3F)nn2)(C(=O)O)C[C@@H]1O ; [N-]=[N+]=NCCCC[C@H](NC(=O)CCCN=[N+]=[N-])C(=O)NCCOCCOCCOCCOCCC(=O)Oc1c(F)c(F)c(F)c(F)c1F ; C#CCOCCOCCO[C@]1(C(=O)O)C[C@H](O)[C@@H](NC(C)=O)[C@H]([C@H](O)[C@H](O)CNC(=O)c2cccc(Oc3ccccc3)c2)O1]

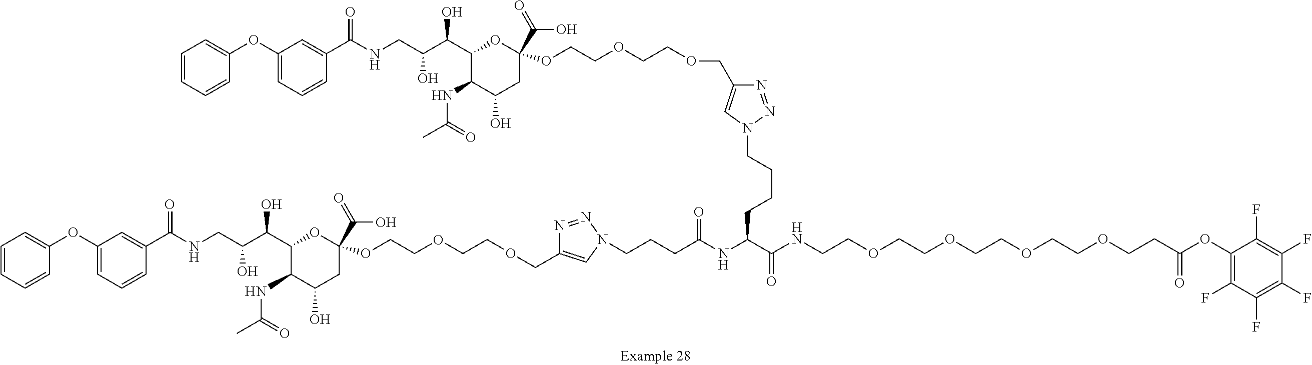 [SMILES: CC(=O)N[C@H]1[C@H]([C@H](O)[C@H](O)CNC(=O)c2cccc(Oc3ccccc3)c2)O[C@@](OCCOCCOCc2cn(CCCC[C@H](NC(=O)CCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)NCCOCCOCCOCCOCCC(=O)Oc3c(F)c(F)c(F)c(F)c3F)nn2)(C(=O)O)C[C@@H]1O]

### Synthesis of (2R,4S,5R,6R)-5-acetamido-2-(2-(2-((1-((S)-18-(4-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)butanamido)-1,17-dioxo-1-(perfluorophenoxy)-4,7,10,13-tetraoxa-16-azadocosan-22-yl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 28)

To a stirred solution of perfluorophenyl (R)-23-azido-18-(4-azidobutyl)-17,20-dioxo-4,7,10,13-tetraoxa-16,19-diazatricosanoate (26594, 1.00 eq) and (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 3.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,4S,5R,6R)-5-acetamido-2-(2-(2-((1-((S)-18-(4-(4-((2-(2-(((2R,4S,5R,6R)-5-acetamido-2-carboxy-6-((1R,2R)-1,2-dihydroxy-3-(3-phenoxybenzamido)propyl)-4-hydroxytetrahydro-2H-pyran-2-yl)oxy)ethoxy)ethoxy)methyl)-1H-1,2,3-triazol-1-yl)butanamido)-1,17-dioxo-1-(perfluorophenoxy)-4,7,10,13-tetraoxa-16-azadocosan-22-yl)-1H-1,2,3-triazol-4-yl)methoxy)ethoxy)ethoxy)-6-((1R,2R)-1,2-dihydroxy-3-(3-phenoxybenzamido)propyl)-4-hydroxytetrahydro-2H-pyran-2-carboxylic acid (Example 28).

### Example 29

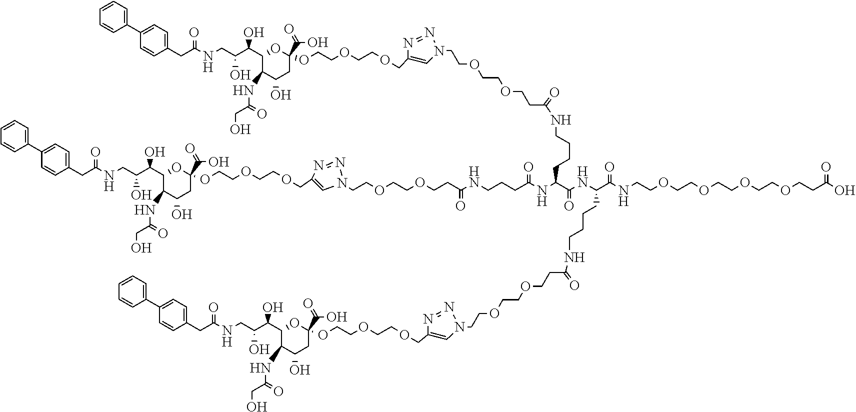 [SMILES: O=C(O)CCOCCOCCOCCOCCNC(=O)[C@H](CCCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NC(=O)[C@H](CCCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NC(=O)CCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1]

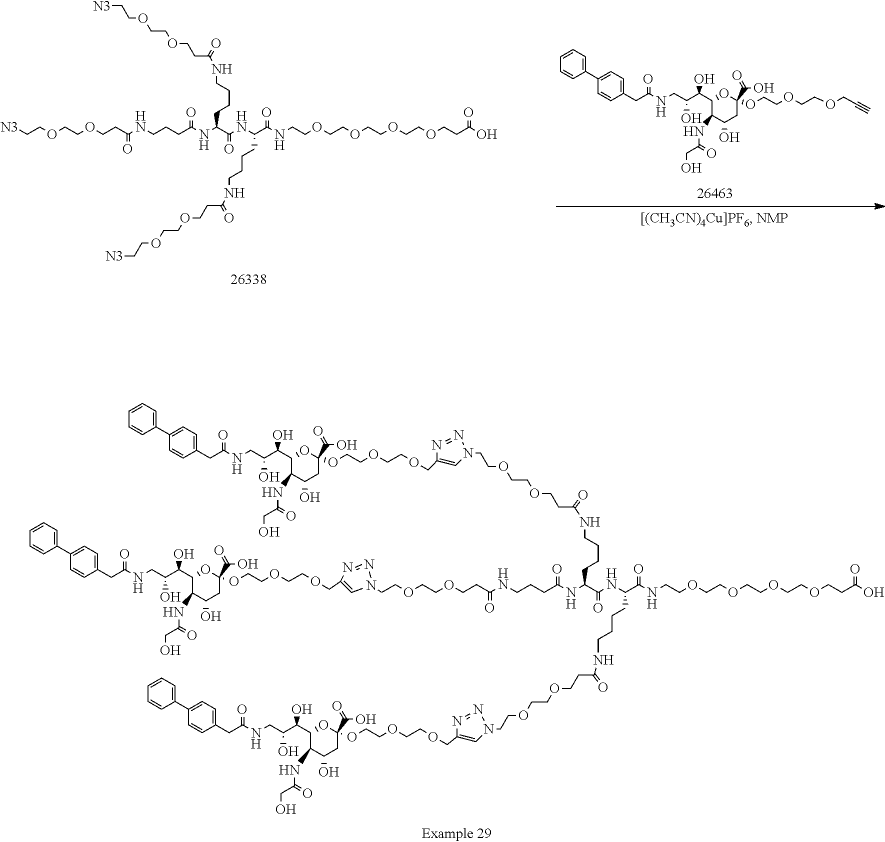

### Synthesis of Example 29

To a stirred solution of (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oic acid (26338, 1.00 eq) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford Example 29.

### Example 30

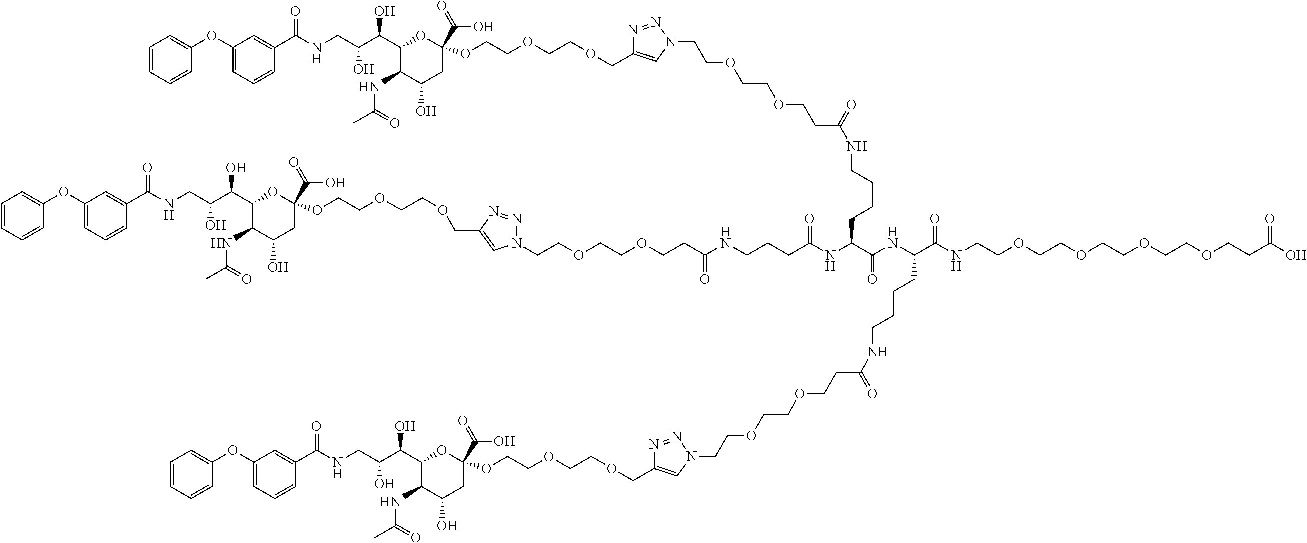 [SMILES: CC(=O)N[C@H]1[C@H]([C@H](O)[C@H](O)CNC(=O)c2cccc(Oc3ccccc3)c2)O[C@@](OCCOCCOCc2cn(CCOCCOCCC(=O)NCCCC[C@H](NC(=O)CCCNC(=O)CCOCCOCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)N[C@@H](CCCCNC(=O)CCOCCOCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)NCCOCCOCCOCCOCCC(=O)O)nn2)(C(=O)O)C[C@@H]1O]

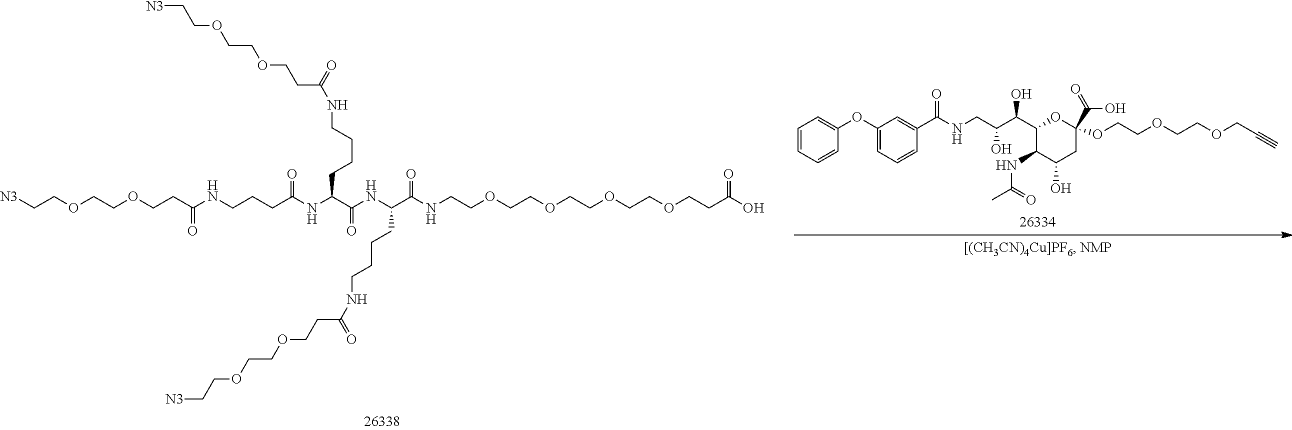 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)O ; C#CCOCCOCCO[C@]1(C(=O)O)C[C@H](O)[C@@H](NC(C)=O)[C@H]([C@H](O)[C@H](O)CNC(=O)c2cccc(Oc3ccccc3)c2)O1]

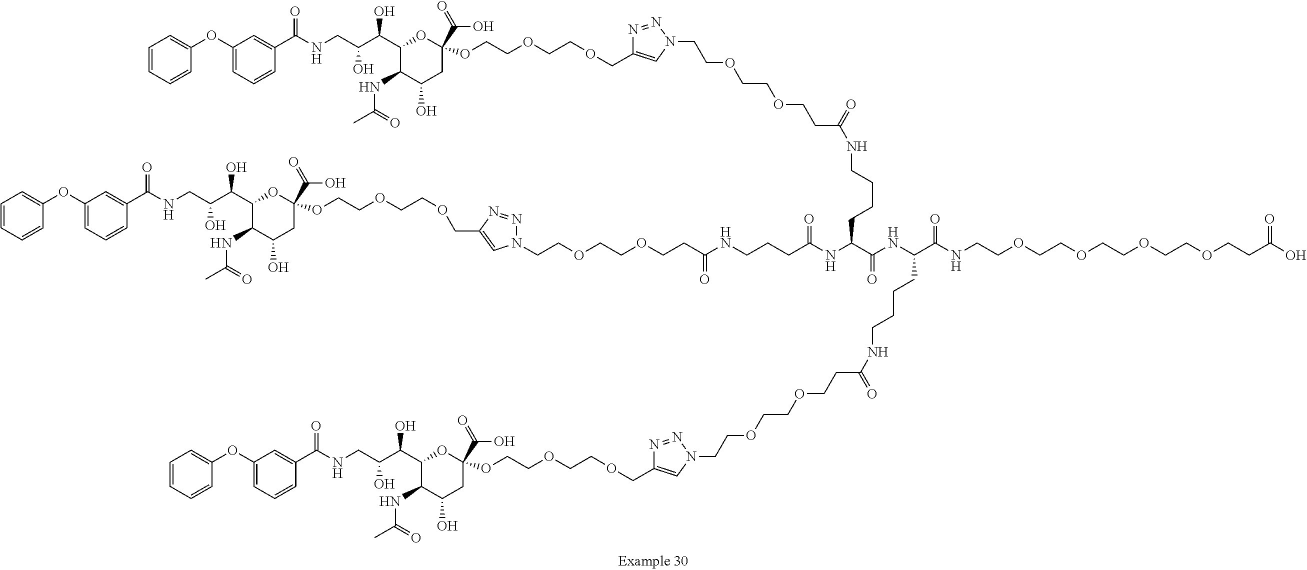 [SMILES: CC(=O)N[C@H]1[C@H]([C@H](O)[C@H](O)CNC(=O)c2cccc(Oc3ccccc3)c2)O[C@@](OCCOCCOCc2cn(CCOCCOCCC(=O)NCCCC[C@H](NC(=O)CCCNC(=O)CCOCCOCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)N[C@@H](CCCCNC(=O)CCOCCOCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)NCCOCCOCCOCCOCCC(=O)O)nn2)(C(=O)O)C[C@@H]1O]

### Synthesis of Example 30

To a stirred solution of (16R,19R)-1-azido-16,19-bis(4-(3-(2-(2-azidoethoxy)ethoxy)propanamido)butyl)-9,14,17,20-tetraoxo-3,6,24,27,30,33-hexaoxa-10,15,18,21-tetraazahexatriacontan-36-oic acid (26338, 1.00 eq) and (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 3.10 eq) in NMP (0.5 mL) in a 1 dram vial is added tetrakis(acetonitrile)copper(I) hexafluorophosphate (7.50 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford Example 30.

### (2R,2′R,4S,4′S,5R,5′R,6R,6′R)-2,2′-(((((((((S)-16-((14-carboxy-3,6,9,12-tetraoxatetradecyl)carbamoyl)-9,14,22-trioxo-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Example 31)

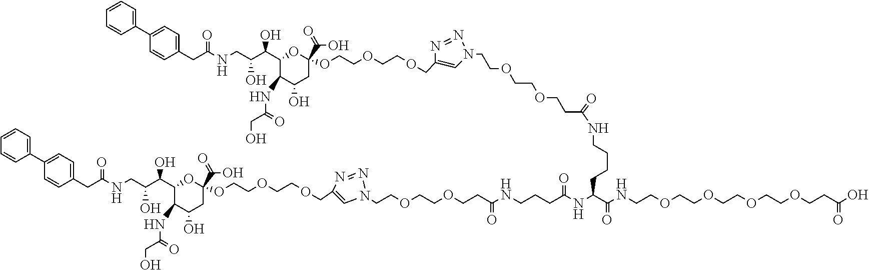 [SMILES: O=C(O)CCOCCOCCOCCOCCNC(=O)[C@H](CCCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NC(=O)CCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1]

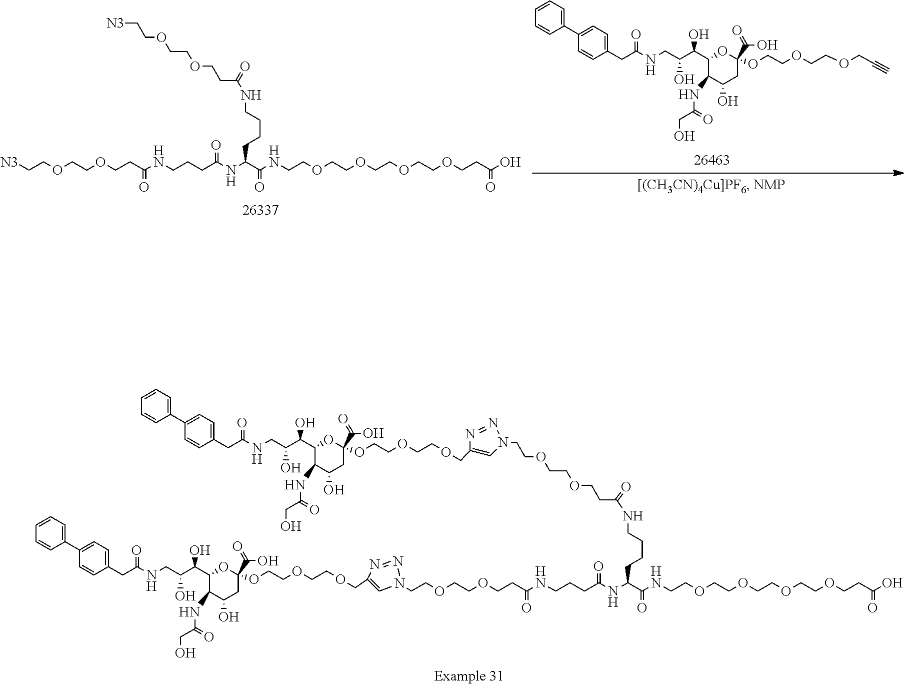 [SMILES: [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)O ; C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1 ; O=C(O)CCOCCOCCOCCOCCNC(=O)[C@H](CCCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NC(=O)CCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1]

### Synthesis of (2R,2′R,4S,4′S,5R,5′R,6R,6′R)-2,2′-(((((((((S)-16-((14-carboxy-3,6,9,12-tetraoxatetradecyl)carbamoyl)-9,14,22-trioxo-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Example 31)

To a stirred solution of (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oic acid (26337, 1.00 eq) and (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (26463, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,2′R,4S,4′S,5R,5′R,6R,6′R)-2,2′-(((((((((S)-16-((14-carboxy-3,6,9,12-tetraoxatetradecyl)carbamoyl)-9,14,22-trioxo-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(6-((1R,2R)-3-(2-([1,1′-biphenyl]-4-yl)acetamido)-1,2-dihydroxypropyl)-4-hydroxy-5-(2-hydroxyacetamido)tetrahydro-2H-pyran-2-carboxylic acid) (Example 31).

### (2R,2′R,4S,4′S,5R,5′R,6R,6′R)-2,2′-(((((((((S)-16-((14-carboxy-3,6,9,12-tetraoxatetradecyl)carbamoyl)-9,14,22-trioxo-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(5-acetamido-6-((1R,2R)-1,2-dihydroxy-3-(3-phenoxybenzamido)propyl)-4-hydroxytetrahydro-2H-pyran-2-carboxylic acid) (Example 32)

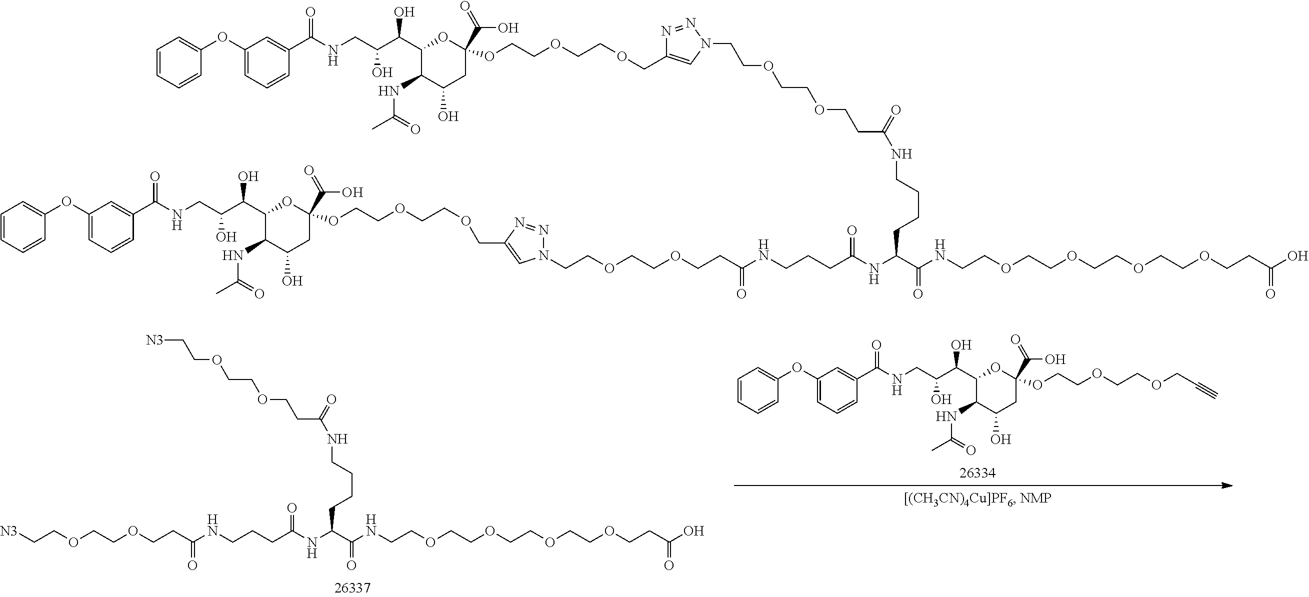 [SMILES: CC(=O)N[C@H]1[C@H]([C@H](O)[C@H](O)CNC(=O)c2cccc(Oc3ccccc3)c2)O[C@@](OCCOCCOCc2cn(CCOCCOCCC(=O)NCCCC[C@H](NC(=O)CCCNC(=O)CCOCCOCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)NCCOCCOCCOCCOCCC(=O)O)nn2)(C(=O)O)C[C@@H]1O ; [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)O ; C#CCOCCOCCO[C@]1(C(=O)O)C[C@H](O)[C@@H](NC(C)=O)[C@H]([C@H](O)[C@H](O)CNC(=O)c2cccc(Oc3ccccc3)c2)O1]

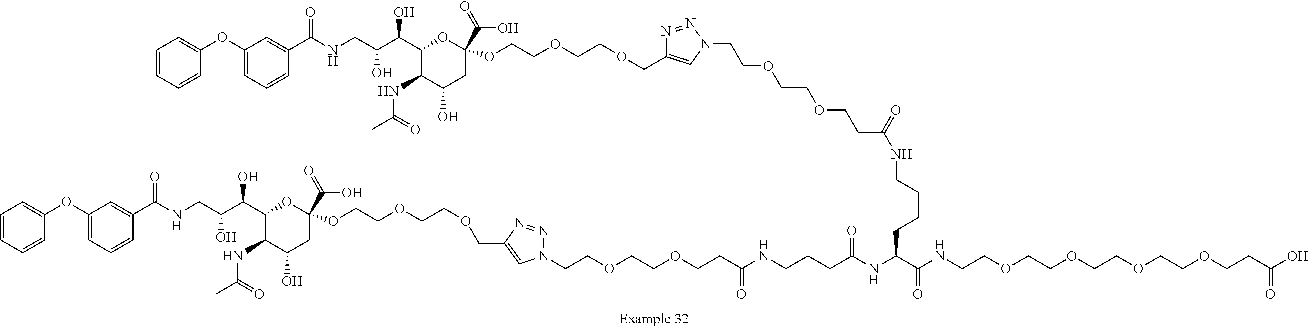 [SMILES: CC(=O)N[C@H]1[C@H]([C@H](O)[C@H](O)CNC(=O)c2cccc(Oc3ccccc3)c2)O[C@@](OCCOCCOCc2cn(CCOCCOCCC(=O)NCCCC[C@H](NC(=O)CCCNC(=O)CCOCCOCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)C(=O)NCCOCCOCCOCCOCCC(=O)O)nn2)(C(=O)O)C[C@@H]1O]

### Synthesis of (2R,2′R,4S,4′S,5R,5′R,6R,6′R)-2,2′-(((((((((S)-16-((14-carboxy-3,6,9,12-tetraoxatetradecyl)carbamoyl)-9,14,22-trioxo-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(5-acetamido-6-((1R,2R)-1,2-dihydroxy-3-(3-phenoxybenzamido)propyl)-4-hydroxytetrahydro-2H-pyran-2-carboxylic acid) (Example 32)

To a stirred solution of (R)-1-azido-16-(4-(3-(2-(2-azidoethoxy)ethoxy)propanamido)butyl)-9,14,17-trioxo-3,6,21,24,27,30-hexaoxa-10,15,18-triazatritriacontan-33-oic acid (26337, 1.00 eq) and (2R,4S,5R,6R)-5-acetamido-6-((1R,2R)-1,2-dihydroxy-3-(3-phenoxybenzamido)propyl)-4-hydroxy-2-(2-(2-(prop-2-yn-1-yloxy)ethoxy)ethoxy)tetrahydro-2H-pyran-2-carboxylic acid (26334, 2.10 eq) in NMP (0.5 mL) in a 1 dram vial is added tetrakis(acetonitrile)copper(I) hexafluorophosphate (5.00 eq). The resulting solution is capped and stirred at room temperature for 10 min. The reaction is diluted with a mixture of 70% acetic acid in NMP, filtered, and purified via preparatory HPLC (15-65% acetonitrile in water with 0.1% TFA). Fractions containing the desired product are combined and lyophilized to dryness to afford (2R,2′R,4S,4′S,5R,5′R,6R,6′R)-2,2′-(((((((((S)-16-((14-carboxy-3,6,9,12-tetraoxatetradecyl)carbamoyl)-9,14,22-trioxo-3,6,25,28-tetraoxa-10,15,21-triazatriacontane-1,30-diyl)bis(1H-1,2,3-triazole-1,4-diyl))bis(methylene))bis(oxy))bis(ethane-2,1-diyl))bis(oxy))bis(ethane-2,1-diyl))bis(oxy))bis(5-acetamido-6-((1R,2R)-1,2-dihydroxy-3-(3-phenoxybenzamido)propyl)-4-hydroxytetrahydro-2H-pyran-2-carboxylic acid) (Example 32).

### perfluorophenyl N2-(4-(3-(2-(2-azidoethoxy)ethoxy)propanamido)butanoyl)-N6-(3-(2-(2-azidoethoxy)ethoxy)propanoyl)-L-lysinate (Cpd. No. ISP6-062)

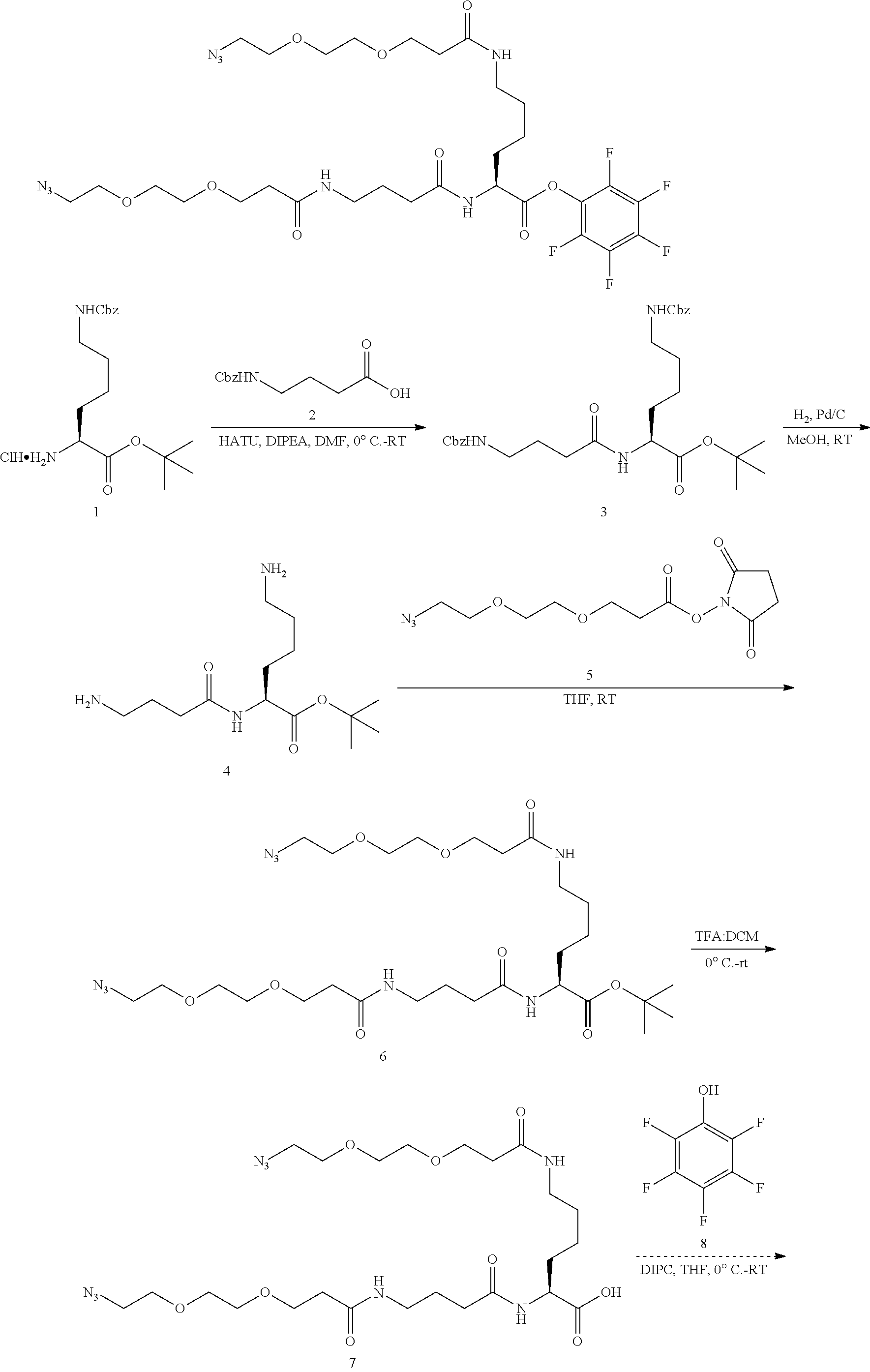

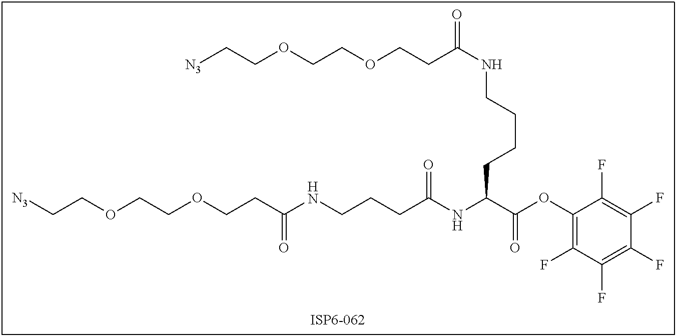 [SMILES: [N-]=[N+]=NCCOCCOCCC(=O)NCCCC[C@H](NC(=O)CCCNC(=O)CCOCCOCCN=[N+]=[N-])C(=O)Oc1c(F)c(F)c(F)c(F)c1F]

### Synthesis of tert-butyl N6-((benzyloxy)carbonyl)-N2-(4-(((benzyloxy)carbonyl)amino)butanoyl)-L-lysinate (3)

To a stirred solution of methyl N6-((benzyloxy)carbonyl)-L-lysinate hydrochloride (1, 10.0 g, 29.7 mmol) and 4-(((benzyloxy)carbonyl)amino)butanoic acid (2, 5.88 g, 24.8 mmol) in N,N-dimethylformamide (50.0 mL) is added [(dimethylamino)({3H-[1,2,3]triazolo[4,5-b]pyridin-3-yloxy})methylidene]dimethylazanium; hexafluoro-λ⁵-phosphanuide (11.3 g, 29.7 mmol) and diisopropylethylamine (8.63 ml, 49.5 mmol). The reaction mixture is stirred at room temperature for 16 h. After completion, the reaction mixture is diluted with saturated sodium bicarbonate solution and extracted with dichloromethane. The organic layer is dried over sodium sulfate, filtered, and concentrated under high vacuum to obtain a crude residue which is then purified via flash column chromatography (50-70% ethyl acetate in hexanes). Desired fractions are concentrated under reduced pressure to afford tert-butyl N6-((benzyloxy)carbonyl)-N2-(4-(((benzyloxy)carbonyl)amino)butanoyl)-L-lysinate (3) as an off white solid. Yield: 11.0 g, 79.7%; LC-MS m/z 556.3 [M+1]⁺.

### Synthesis of tert-butyl (4-aminobutanoyl)-L-lysinate (4)

To a stirred solution of tert-butyl N6-((benzyloxy)carbonyl)-N2-(4-(((benzyloxy)carbonyl)amino)butanoyl)-L-lysinate (3, 8.0 g, 14.4 mmol) in Methanol (250.0 mL) is added 10% palladium on carbon (3.50 g) at room temperature under nitrogen. The resulting mixture is stirred at room temperature under hydrogen gas pressure for 12 h. The reaction mixture is filtered through celite and washed with methanol. The filtrate is concentrated under vacuum to afford tert-butyl (4-aminobutanoyl)-L-lysinate (4) as a pale yellow viscous liquid. Yield: 4.0 g, 95%; ELSD m/z 288.4 [M+1]⁺.

### Synthesis of tert-butyl N2-(4-(3-(2-(2-azidoethoxy)ethoxy)propanamido)butanoyl)-N6-(3-(2-(2-azidoethoxy)ethoxy)propanoyl)-L-lysinate (6)

To a stirred solution of tert-butyl (4-aminobutanoyl)-L-lysinate (4, 1.0 g, 3.48 mmol) in tetrahydrofuran (20.0 mL) at 0° C. is added 2,5-dioxopyrrolidin-1-yl 3-(2-(2-azidoethoxy)ethoxy)propanoate (5, 2.09 g, 6.96 mmol). The resulting reaction mixture is stirred at room temperature for 4 h. After completion, the solvent is concentrated under high vacuum to obtain a crude residue which is then purified via flash column chromatography (0-7.5% methanol in dichloromethane). Desired fractions are concentrated under reduced pressure to afford tert-butyl N2-(4-(3-(2-(2-azidoethoxy)ethoxy)propanamido)butanoyl)-N6-(3-(2-(2-azidoethoxy)ethoxy)propanoyl)-L-lysinate (6) as a pale yellow viscous liquid Yield: 1.20 g, 52%; ELSD m/z 658.2 [M+1]⁺.

### Synthesis of N2-(4-(3-(2-(2-azidoethoxy)ethoxy)propanamido)butanoyl)-N6-(3-(2-(2-azidoethoxy)ethoxy)propanoyl)-L-lysine (7)

To a stirred solution of tert-butyl N2-(4-(3-(2-(2-azidoethoxy)ethoxy)propanamido)butanoyl)-N6-(3-(2-(2-azidoethoxy)ethoxy)propanoyl)-L-lysinate (6, 0.40 g, 0.608 mmol) in dichloromethane (5 mL) is added trifluoroacetic acid (1.0 mL) at 0° C. The resulting mixture is stirred at room temperature under nitrogen for 4 h. After completion, the reaction mixture is concentrated, washed with diethyl ether, and dried to afford N2-(4-(3-(2-(2-azidoethoxy)ethoxy)propanamido)butanoyl)-N6-(3-(2-(2-azidoethoxy)ethoxy)propanoyl)-L-lysine (7) as a pale yellow viscous liquid. Yield: 0.350 g, 96%; ELSD m/z 602.4 [M+1]⁺.

### Synthesis of perfluorophenyl N2-(4-(3-(2-(2-azidoethoxy)ethoxy)propanamido)butanoyl)-N6-(3-(2-(2-azidoethoxy)ethoxy)propanoyl)-L-lysinate (Cpd. No. ISP6-062)

To a stirred solution of N2-(4-(3-(2-(2-azidoethoxy)ethoxy)propanamido)butanoyl)-N6-(3-(2-(2-azidoethoxy)ethoxy)propanoyl)-L-lysine (7, 1.0 eq) in tetrahydrofuran at 0° C. is added 2,3,4,5,6-pentafluorophenol (8, 2.0 eq) and N, N′-diisopropylcarbodiimide (2.5 eq). The resulting reaction solution is stirred at room temperature. After completion, the solvent is concentrated to afford a crude residue which is then purified via preparatory HPLC. Fractions containing the desired product are combined and lyophilize to dryness to afford perfluorophenyl N2-(4-(3-(2-(2-azidoethoxy)ethoxy)propanamido)butanoyl)-N6-(3-(2-(2-azidoethoxy)ethoxy)propanoyl)-L-lysinate (Cpd. No. ISP6-062).

### Perfluorophenyl N2-(N2-(4-(3-(2-(2-azidoethoxy)ethoxy)propanamido)butanoyl)-N6-(3-(2-(2-azidoethoxy)ethoxy)propanoyl)-L-lysyl)-N6-(3-(2-(2-azidoethoxy)ethoxy)propanoyl)-L-lysinate (Cpd. No. ISP6-065)

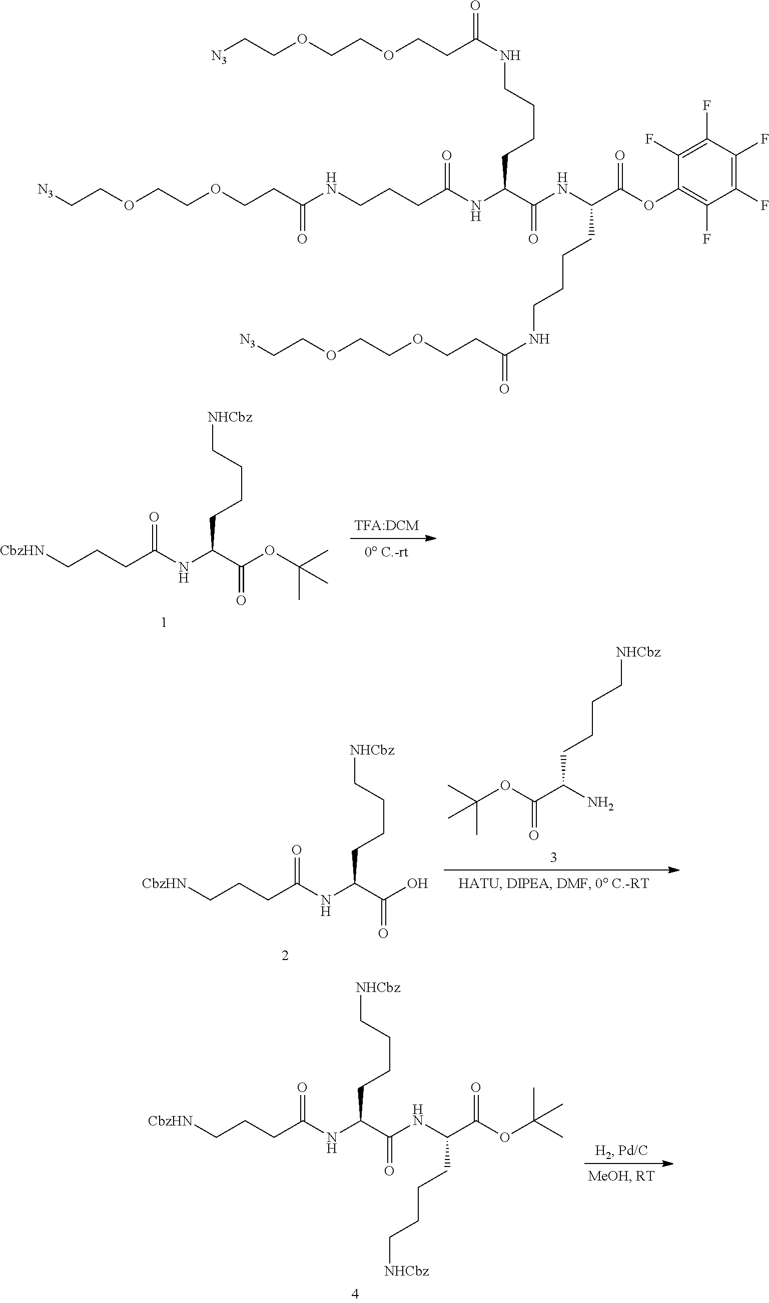 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)Oc1c(F)c(F)c(F)c(F)c1F ; CC(C)(C)OC(=O)[C@H](CCCCNC(=O)OCc1ccccc1)NC(=O)CCCNC(=O)OCc1ccccc1 ; O=C(CCCNC(=O)OCc1ccccc1)N[C@@H](CCCCNC(=O)OCc1ccccc1)C(=O)O ; CC(C)(C)OC(=O)[C@@H](N)CCCCNC(=O)OCc1ccccc1 ; CC(C)(C)OC(=O)[C@H](CCCCNC(=O)OCc1ccccc1)NC(=O)[C@H](CCCCNC(=O)OCc1ccccc1)NC(=O)CCCNC(=O)OCc1ccccc1]

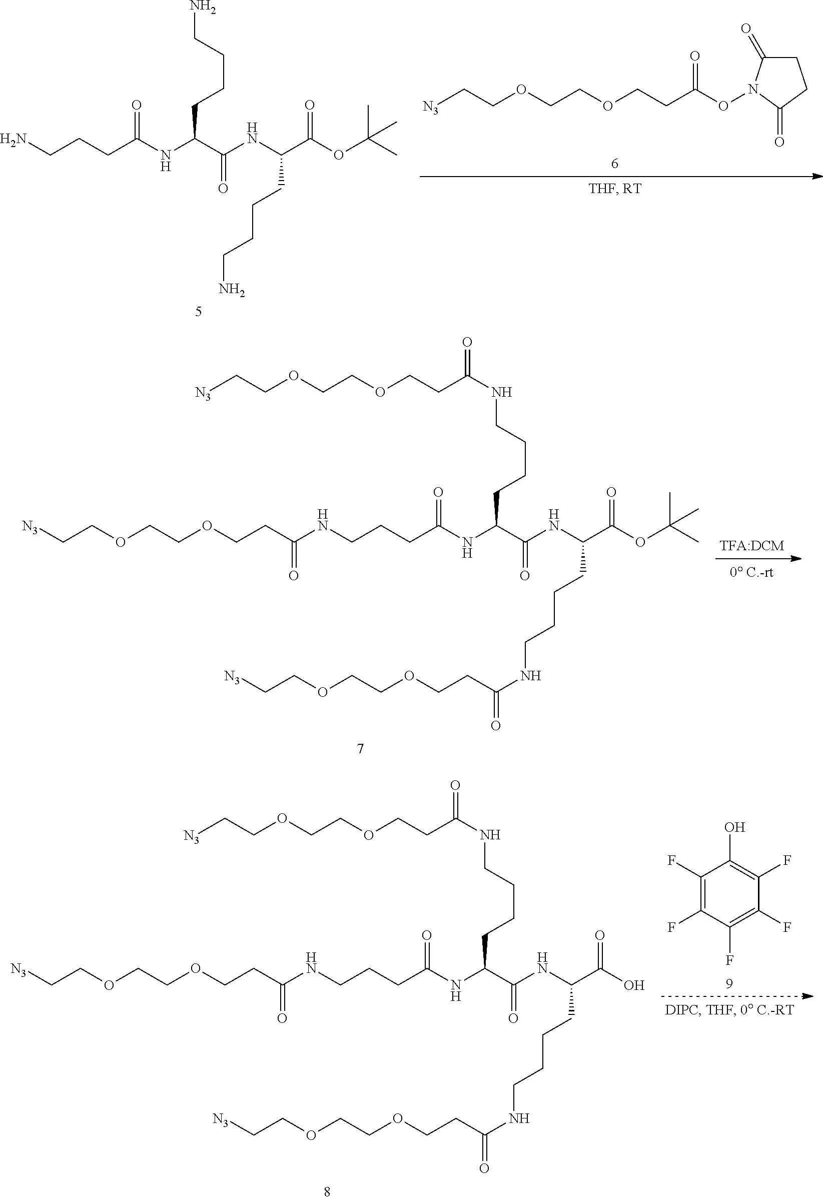

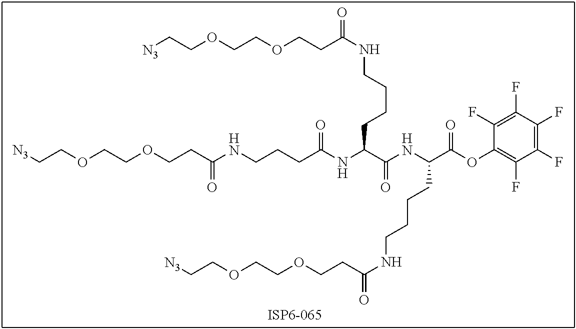 [SMILES: [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)Oc1c(F)c(F)c(F)c(F)c1F]

### Synthesis of N6-((benzyloxy)carbonyl)-N2-(4-(((benzyloxy)carbonyl)amino)butanoyl)-L-lysine (2)

To a stirred solution of tert-butyl N6-((benzyloxy)carbonyl)-N2-(4-(((benzyloxy)carbonyl)amino)butanoyl)-L-lysinate (1, 5.0 g, 9.0 mmol) in dichloromethane (50.0 mL) is added trifluoroacetic acid (10.0 mL) at 0° C. The resulting mixture is stirred at room temperature under nitrogen for 6 h. After completion, the reaction mixture is concentrated, washed with diethyl ether, and dried to afford N6-((benzyloxy)carbonyl)-N2-(4-(((benzyloxy)carbonyl)amino)butanoyl)-L-lysine (2) as an off white solid. Yield: 3.8 g, 85%; LC-MS m/z 500.1 [M+1]⁺.

### Synthesis of tert-butyl N6-((benzyloxy)carbonyl)-N2-(N6-((benzyloxy)carbonyl)-N2-(4-(((benzyloxy)carbonyl)amino)butanoyl)-L-lysyl)-L-lysinate (4)

To a stirred solution of N6-((benzyloxy)carbonyl)-N2-(4-(((benzyloxy)carbonyl)amino)butanoyl)-L-lysine (2, 3.80 g, 7.61 mmol) and tert-butyl N6-((benzyloxy)carbonyl)-L-lysinate hydrochloride (3, 3.38 g, 9.13 mmol) in N,N-dimethylformamide (50.0 mL) is added [(dimethylamino)({3H-[1,2,3]triazolo[4,5-b]pyridin-3-yloxy})methylidene]dimethylazanium; hexafluoro-λ⁵-phosphanuide (3.47 g, 9.13 mmol) and diisopropylethylamine (3.32 ml, 19 mmol). The reaction mixture is stirred at room temperature for 16 h. After completion, the reaction mixture is diluted with saturated sodium bicarbonate solution and extracted with dichloromethane. The organic layer is dried over sodium sulfate, filtered, and concentrated under high vacuum to obtain a crude residue which is then purified via flash column chromatography (70-100% ethyl acetate in hexanes). Desired fractions are concentrated under reduced pressure to afford tert-butyl N6-((benzyloxy)carbonyl)-N2-(N6-((benzyloxy)carbonyl)-N2-(4-(((benzyloxy)carbonyl)amino) butanoyl)-L-lysyl)-L-lysinate (4) as an off white solid. Yield: 5.70 g, 90%; LC-MS m/z 818.4 [M+1]⁺.

### Synthesis of tert-butyl (4-aminobutanoyl)-L-lysyl-L-lysinate (5)

To a stirred solution of tert-butyl N6-((benzyloxy)carbonyl)-N2-(N6-((benzyloxy)carbonyl)-N2-(4-(((benzyloxy)carbonyl) amino) butanoyl)-L-lysyl)-L-lysinate (4, 4.0 g, 4.89 mmol) in Methanol (100 mL) is added 10% palladium on carbon (1.5 g) at room temperature under nitrogen. The resulting mixture is stirred at room temperature under hydrogen gas pressure for 12 h. The reaction mixture is filtered through celite and washed with methanol. The filtrate is concentrated under vacuum to afford tert-butyl (4-aminobutanoyl)-L-lysyl-L-lysinate (5) as a pale yellow viscous liquid. Yield: 2.0 g, 98%; ELSD m/z 416.2 [M+1]⁺.

### Synthesis of tert-butyl N2-(N2-(4-(3-(2-(2-azidoethoxy)ethoxy)propanamido)butanoyl)-N6-(3-(2-(2-azidoethoxy)ethoxy)propanoyl)-L-lysyl)-N6-(3-(2-(2-azidoethoxy)ethoxy)propanoyl)-L-lysinate (7)

To a stirred solution of tert-butyl (4-aminobutanoyl)-L-lysyl-L-lysinate (5, 1.0 g, 2.41 mmol) in tetrahydrofuran (20.0 mL) at 0° C. is added 2,5-dioxopyrrolidin-1-yl 3-(2-(2-azidoethoxy)ethoxy)propanoate (6, 2.17 g, 7.22 mmol). The resulting reaction mixture is stirred at room temperature for 4 h. After completion, the solvent is concentrated under high vacuum to obtain a crude residue which is then purified via flash column chromatography (0-7.5% methanol in dichloromethane). Desired fractions are concentrated under reduced pressure to afford tert-butyl N2-(N2-(4-(3-(2-(2-azidoethoxy)ethoxy)propanamido)butanoyl)-N6-(3-(2-(2-azidoethoxy)ethoxy)propanoyl)-L-lysyl)-N6-(3-(2-(2-azidoethoxy)ethoxy)propanoyl)-L-lysinate (7) as a colorless viscous liquid. Yield: 1.30 g, 55%; ELSD m/z 971.8 [M+1]⁺.

### Synthesis of N2-(N2-(4-(3-(2-(2-azidoethoxy)ethoxy)propanamido)butanoyl)-N6-(3-(2-(2-azidoethoxy)ethoxy)propanoyl)-L-lysyl)-N6-(3-(2-(2-azidoethoxy)ethoxy)propanoyl)-L-lysine (8)

To a stirred solution of tert-butyl N2-(N2-(4-(3-(2-(2-azidoethoxy)ethoxy)propanamido)butanoyl)-N6-(3-(2-(2-azidoethoxy)ethoxy)propanoyl)-L-lysyl)-N6-(3-(2-(2-azidoethoxy)ethoxy)propanoyl)-L-lysinate (7, 0.180 g, 0.185 mmol) in dichloromethane (2 mL) is added trifluoroacetic acid (0.3 mL) at 0° C. The resulting mixture is stirred at room temperature under nitrogen for 6 h. After completion, the reaction mixture is concentrated, washed with diethyl ether, and dried to afford N2-(N2-(4-(3-(2-(2-azidoethoxy)ethoxy)propanamido)butanoyl)-N6-(3-(2-(2-azidoethoxy)ethoxy)propanoyl)-L-lysyl)-N6-(3-(2-(2-azidoethoxy)ethoxy)propanoyl)-L-lysine (8) as a pale yellow viscous liquid. Yield: 0.170 g, 98%; ELSD m/z 915.5 [M+1]⁺.

### Synthesis of Perfluorophenyl N2-(N2-(4-(3-(2-(2-azidoethoxy)ethoxy)propanamido)butanoyl)-N6-(3-(2-(2-azidoethoxy)ethoxy)propanoyl)-L-lysyl)-N6-(3-(2-(2-azidoethoxy)ethoxy)propanoyl)-L-lysinate (Cpd. No. ISP6-065)

To a stirred solution of N2-(N2-(4-(3-(2-(2-azidoethoxy)ethoxy)propanamido)butanoyl)-N6-(3-(2-(2-azidoethoxy)ethoxy)propanoyl)-L-lysyl)-N6-(3-(2-(2-azidoethoxy)ethoxy)propanoyl)-L-lysine (8, 1.0 eq) in tetrahydrofuran at 0° C. is added 2,3,4,5,6-pentafluorophenol (9, 2.0 eq) and N, N′-diisopropylcarbodiimide (2.5 eq). The resulting reaction solution is stirred at room temperature. After completion, the solvent is concentrated and purified via preparatory HPLC. Fractions containing the desired product are combined and lyophilize to dryness to afford Perfluorophenyl N2-(N2-(4-(3-(2-(2-azidoethoxy)ethoxy)propanamido)butanoyl)-N6-(3-(2-(2-azidoethoxy)ethoxy)propanoyl)-L-lysyl)-N6-(3-(2-(2-azidoethoxy)ethoxy)propanoyl)-L-lysinate (Cpd. No. ISP6-065).

Linkers structures used in conjugates described in the examples are listed in Table 1.

TABLE 1
SIGLEC LIGAND-LINKERS

| | | Human Siglec-2 | Mouse Siglec-2 | | Linker |
|---|---|---|---|---|---|
| Compound | Alias | Binding Affinity | Binding Affinity | Valency | Structure |
| 26288 | BPC-Neu5Gc Monovalent PEG | Strong | Strong | Mono | PEG |
| 26415 | BPC-Neu5Gc Monovalent GAL | Strong | Strong | Mono | GAL |
| 26475 | BPC-Neu5Gc Bivalent PEG | Strong | Strong | Bi | PEG |
| 26416 | BPC-Neu5Gc Bivalent GAL | Strong | Strong | Bi | GAL |
| 26477 | BPC-Neu5Gc Trivalent PEG | Strong | Strong | Tri | PEG |
| 26417 | BPC-Neu5Gc Trivalent GAL | Strong | Strong | Tri | GAL |
| 24836 | MPB-Neu5Ac Monovalent PEG | Strong | V. weak | Mono | PEG |
| 24906 | MPB-Neu5Ac Monovalent GAL | Strong | V. weak | Mono | GAL |
| 26289 | MPB-Neu5Ac Bivalent PEG | Strong | V. weak | Bi | PEG |
| 26468 | MPB-Neu5Ac Bivalent GAL | Strong | V. weak | Bi | GAL |
| 26467 | MPB-Neu5Ac Trivalent PEG | Strong | V. weak | Tri | PEG |
| 26471 | MPB-Neu5Ac Trivalent GAL | Strong | V. weak | Tri | GAL |
| 26614 | Neu5Gc Monovalent PEG | Weak | Weak | Mono | PEG |
| 26615 | Neu5Gc Bivalent PEG | Weak | Weak | Bi | PEG |
| 26616 | Neu5Gc Trivalent PEG | Weak | Weak | Tri | PEG |
| 26530 | Asialo Monovalent PEG | No Binding | No Binding | Mono | PEG |
| 26532 | Asialo Bivalent PEG | No Binding | No Binding | Bi | PEG |
| 26534 | Asialo Trivalent PEG | No Binding | No Binding | Tri | PEG |

### Example 6: Binding Analysis of Synthetic Siglec-2/CD22 Ligand Interactions with Siglec-2/CD22 by Surface Plasmon Resonance

### Purpose

To determine the in vitro binding properties of synthetic Siglec ligands for recombinant human and mouse CD22 ectodomains.

### Materials and Methods

The following ligands were evaluated for mouse and human CD22 binding: 1) BPC-Neu5Gc PEG (Cpd. No. 26463), 2) BPC-Neu5Gc GAL (Cpd. No. 26339), 3) MPB-Neu5Ac PEG (Cpd. No. 26334), 4) MPB-Neu5Ac GAL (Cpd. No. 26409), 5) Neu5Gc PEG (Cpd. No. 26591). All tested compounds are alkyne-terminating precursors of PFP-terminating, conjugatable Siglec Linker structures used in conjugates in the following examples.

Binding assays for synthetic CD22 ligand binding to CD22 receptor ectodomain were run on a Biacore 8K+ instrument (Cytiva). Surface preparation for CD22 conjugates binding to human or mouse CD22 protein consisted of two steps, covalent immobilization of streptavidin followed by capture of Avi-tagged and biotinylated human CD22-Fc fusion (R&D Systems, Cat #AV11968-050) and a mono-Fc fusion of mouse CD22. Using standard amine coupling protocols, Streptavidin (Invitrogen, Cat #: 434301) was immobilized to a Cytiva CM5 chip (Cytiva, Cat #BR100530) by injecting at 100 ug/mL in Sodium Acetate, pH 4.5 (Cytiva, Cat #BR100350) on both flow cells, yielding a final response of 2500 RU. Capture of biotinylated mono Fc human CD22 was performed on channels 1-4 and mouse CD22 on channels 5-8 on the active flow cell (2) and the reference flow cell (1) was kept as unmodified streptavidin to account for any non-specific binding. Human CD22 or mouse CD22 (mono-Fc fusion, 5 μg/mL) was injected on the active flow cell (2) for 90 seconds at 5 μL/min, yielding about 210 RU of captured constructs.

Binding experiments of Siglec-ligand conjugated proteins were performed on the surfaces prepared above in HBS-EP+ (10 mM HEPES, 150 mM NaCl, 3 mM EDTA, 0.05% Tween-20, pH 7.5) as the running buffer. Conjugates were serially diluted 1:1 in running buffer from 1 μM to 31.25 nM and injected over both the reference and active flow cells for 90 seconds at 30 μL/min. The conjugates were then allowed to dissociate from the surface for 300 seconds. No regeneration was required as the conjugates completely dissociated from the surface after 30 seconds.

### Results and Conclusions

Affinity determinations are summarized in TABLE 2. As expected, so-called BPC-Neu5Gc- and MPB-Neu5Ac-based Siglec ligand structures bind to recombinant human CD22 more tightly than Sialic acid-based Neu5Gc-based Siglec ligand structures. Ligand structures based on a PEG linker vs a GAL linker do not differ in binding affinity. Also as expected, MPB-Neu5Ac-based structures bind tightly to human CD22 and bind only very weakly to mouse CD22. BPC-Neu5Gc and MPB-Neu5Ac can thus form the basis for Siglec-2/CD22 Ligand conjugates potentiated for Siglec-2 binding.

TABLE 2
SIGLEC LIGAND AFFINITIES FOR HUMAN
& MOUSE CD22/SIGLEC-2 ECTODOMAINS

| | | Human Siglec-2 | Mouse Siglec-2 | |
|---|---|---|---|---|
| | | Binding Affinity | Binding Affinity | Linker |
| Compound | Alias | (μM) | (μM) | Structure |
| 26463 | BPC-Neu5Gc PEG | 1.3 | 9.6 | PEG |
| 26339 | BPC-Neu5Gc GAL | 2.5 | 17.2 | GAL |
| 26334 | MPB-Neu5Ac PEG | 0.93 | >100 | PEG |
| 26409 | MPB-Neu5Ac GAL | 1.0 | >100 | GAL |
| 26591 | Neu5Gc PEG | >450 | >1000 | PEG |

### Example 7: Production and Characterization of Protein-Siglec Ligand Conjugates

### Purpose

To produce Siglec ligand-linker conjugates of proteins for evaluation in in vitro B cell signaling assays and in vivo immunogenicity experiments. Proteins were either produced in-house or procured from commercial sources, as described below.

### Materials and Methods

Antibody (Anti-IgD Human IgG1 Chimeric Antibody, Adalimumab Anti-hTNFα hIgG1) Expression, Purification, and Analytics

For antibody expression, the ExpiFectamine 293 Transfection kit (Life Technologies, A14524) was used to transfect suspension Expi293F cells (Life Technologies, A14527) with Heavy Chain and Light Chain plasmids (pTT5-based) at a 1:1 ratio. Media was harvested 3-6 days post-transfection by centrifugation and filtered using 0.2 μm PES vacuum sterile single-use filter unit (ThermoScientific, 5670020).

Purification was performed with 1.5 mL MabSelect Sure resin (Cytiva/GE Cat #: 17-5438-03) for each 250 mL culture supernatant. Briefly, each column was equilibrated with PBS pH 7.2 and loaded with culture supernatant. After the loading step, the column was washed with PBS pH 7.2 and eluted with 10 mL IgG Elution buffer (Thermo Scientific Ref 21004). The pH of the elution pool was adjusted with 1 mL 1 M Sodium Phosphate pH 6.5 for each 10 mL elution pool. Finally, buffer exchange was performed with PBS pH 7.2 using a 30 kDa Amicon Ultra-15 Centrifugal Filter Unit.

Analysis of endotoxin content was performed using the Charles River Endosafe PTS 0.01-1 EU/ml detection. Size exclusion chromatography was performed on an Agilent Chemstation HPLC-SEC with a Sepax-Zenix SEC-300, 200 mm×7.8 mm ID, 3 uM column. Capillary gel electrophoresis (cGE) was performed on a Caliper LabChip GXII Protein 200 with the Perkin Elmer Chip (Cat #760499). LC-MS analysis was performed on SciEX LC 5600+, ExionLC AD, Analyst TF 1.8.1 with an Agilent AdvanceBio Desalting-RP, Column 1000A, 10 um.

Expression and Purification of E. coli L-Asparaginase

The DNA sequence corresponding to L-asparaginase 2 from E. coli (aa L23-Y348, uniprot P00805) was cloned into a pET plasmid containing an N-terminal His tag. The construct was transformed into ClearColi BL21(DE3) electrocompetent cells (Lucigen, 60810-1) and grown overnight in Miller's Luria Broth (LB) containing 100 μg/mL ampicillin. Next, overnight cultures were diluted 1:50 in fresh LB containing 100 μg/mL ampicillin and grown to OD₆₀₀ 1-1.5 at 37° C. Cultures were then induced with 0.1 mM Isopropyl β-d-1-thiogalactopyranoside (IPTG) for 24 hours at 37° C. Cells were pelleted by centrifugation at 4,000 rpm for 20 mL and resuspended in 40 mL of lysis buffer (50 mM sodium phosphate pH 7.86, 200 mM NaCl, 20 mM imidazole, and EDTA-free protease inhibitor cocktail tablet). Resuspended cells were sonicated using a Qsonica sonicator (parameters: pulse ON 1 sec, pulse OFF 2 sec, 2 min, Amplitude 40%). The lysate was clarified by centrifugation at 16,000 rpm for 20 minutes and incubated with Ni-NTA resin (Invitrogen, 60-0442) for 1 hour at 4° C. with end-over-end mixing. The mixture was transferred to a 25 mL column for gravity flow chromatography and washed with wash buffer (50 mM sodium phosphate pH 7.86, 200 mM NaCl, 20 mM imidazole). The protein was eluted with buffer containing 50 mM sodium phosphate pH 7.86, 200 mM NaCl, and 300 mM imidazole and buffer exchange was performed with PBS pH 7.2 using a 10 kDa Amicon Ultra-15 Centrifugal Filter Unit. Endotoxin removal was performed using a Triton X-114 extraction method. Briefly, Triton X-114 was added to the protein solution to a final concentration of 2% v/v. The solution was incubated at 4° C. for 2 hours with end-over-end mixing. Samples were transferred to a 37° C. water bath for 10 minutes, followed by centrifugation at 20,000 g for 20 minutes at room temperature. The top protein layer was separated from the Triton X-114 layer by pipetting. The detergent was removed using HiPPR detergent removal resin (ThermoFisher, 88305) following the manufacturers protocol.

Analysis of endotoxin content was performed using the Charles River Endosafe PTS 0.01-1 EU/ml detection. Size exclusion chromatography was performed on an Agilent Chemstation HPLC-SEC with a Sepax-Zenix SEC-300, 200 mm×7.8 mm ID, 3 uM column. Capillary gel electrophoresis (cGE) was performed on a Caliper LabChip GXII Protein 200 with the Perkin Elmer Chip (Cat #760499). LC-MS analysis was performed on SciEX LC 5600+, ExionLC AD, Analyst TF 1.8.1 with an Agilent AdvanceBio Desalting-RP, Column 1000A, 10 um.

### Protein-Siglec Ligand Conjugation

Pentafluorophenyl (PFP) conjugatable Siglec Ligand linker was added to reaction mixtures at a molar ratio of 4-30 times above protein based on desired degree of labeling in the presence of 10% v/v of 50 mM Sodium Tetraborate pH 8.5 and 10% v/v DMSO. Reactions were incubated for 3 hours at 25° C. After the 3 h incubation period, 10% v/v of 1 M Tris-HCl pH 8.0 was added to quench the unreacted linker-payload. Neutralized reactions were then allowed to incubate at 25° C. for 15 min.

### Protein Siglec-Ligand Conjugate Purification

Quenched conjugation reactions are purified by preparative size exclusion chromatography at 4° C. using either Superdex 200 Increase 10/300 GL or HiLoad 16/600 Superdex 200 pg at a flow rate of 0.75 mL/min, with PBS pH 7.2.

### Analysis of Protein-Siglec Ligand Conjugates

### LC-MS

Mass spectrometer: SciEX LC 5600+, ExionLC AD, Analyst TF 1.8.1
HPLC: Agilent AdvanceBio Desalting-RP, Column 1000A, 10 um
2.1 mm×12.5 mm, flow rate 400 ul/min, Sample load: 5 ug
Buffer A: Water+0.1% Formic Acid
Buffer B: Acetonitrile+0.1% Formic Acid

### Analytical SEC

Superose 6 Increase 10/300 GL
Injection Volume: 25 uL at 1 mg/mL of sample.
Buffer: 50 mM Sodium Phosphate+400 mM Sodium Perchlorate, pH 6.2
Flow rate: 0.7 mL/min
Run time 40 min at 25° C.

### Capillary Gel Electrophoresis

Caliper LabChip GXII Protein 200

### Endotoxin Measurement

Charles River Endosafe PTS Cartridge 0.01-1 EU/ml
Lonza Pyrogene C Endpoint Endotoxin Assay

The “LDR”, or Ligand-to-Drug ratio, was measured for each conjugate preparation by LC/MS, by evaluating the relative abundances of species varying in the degree of conjugation, as described here. Random conjugation methods (to lysine/amines in this case) result in a mixture of species varying in the degree of conjugation per adalimumab species. Such a series of molecular species can be represented as:

$${\sum }_{i = 0}^{m} {[ {X}_{n} L ]}_{i} Y$$

In this statement, each biotherapeutic, Y (adalimumab, in this case), is covalently bound to a Siglec Ligand as defined by XₙL, where X is a sialic acid species of valency, n, with a Siglec Ligand-to-biotherapeutic ratio that varies between 0 and m. All species have the same Sialic Acid valency, n (monovalent, bivalent, or trivalent).

As a total measure of the degree of conjugation in such an ensemble of species with varying degrees of conjugation, the LDR can be defined as follows: LDR is a weighted average of the individual Siglec ligand-to-biotherapeutic ratios (integer value i) in a mixture of species varying in said ratio, and Pᵢ (0≤Pᵢ≤1, with Σᵢ₌₀^{m}Pᵢ=1) representing the fractional abundance of each species in the mixture:

$$L D R = {\sum }_{i = 0}^{m} i {P}_{i}$$

### Results

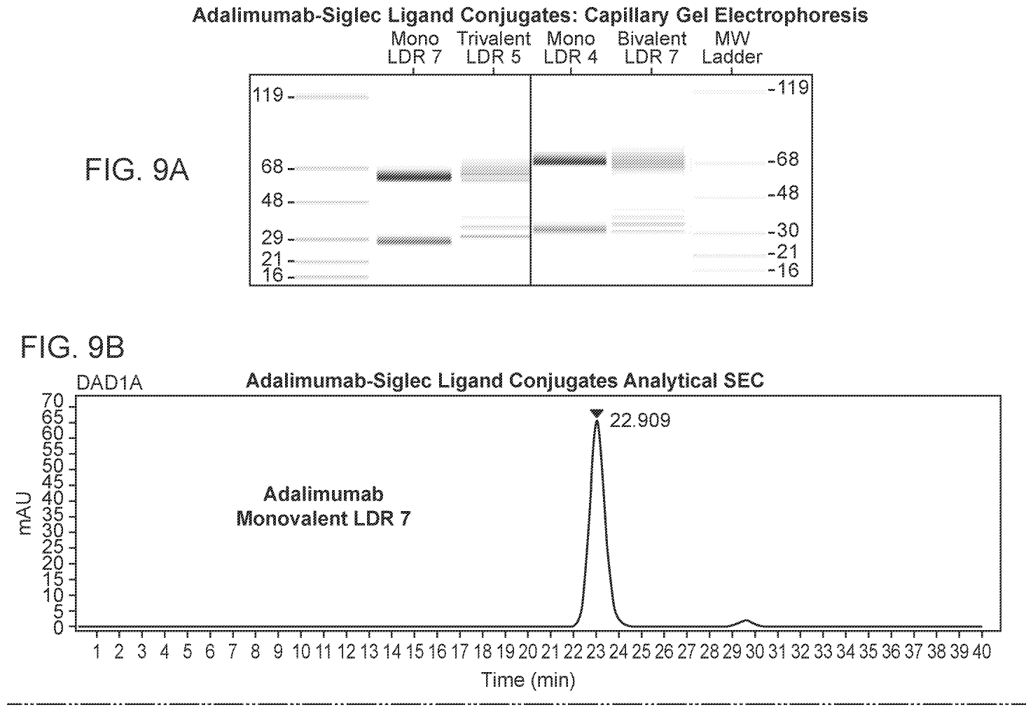

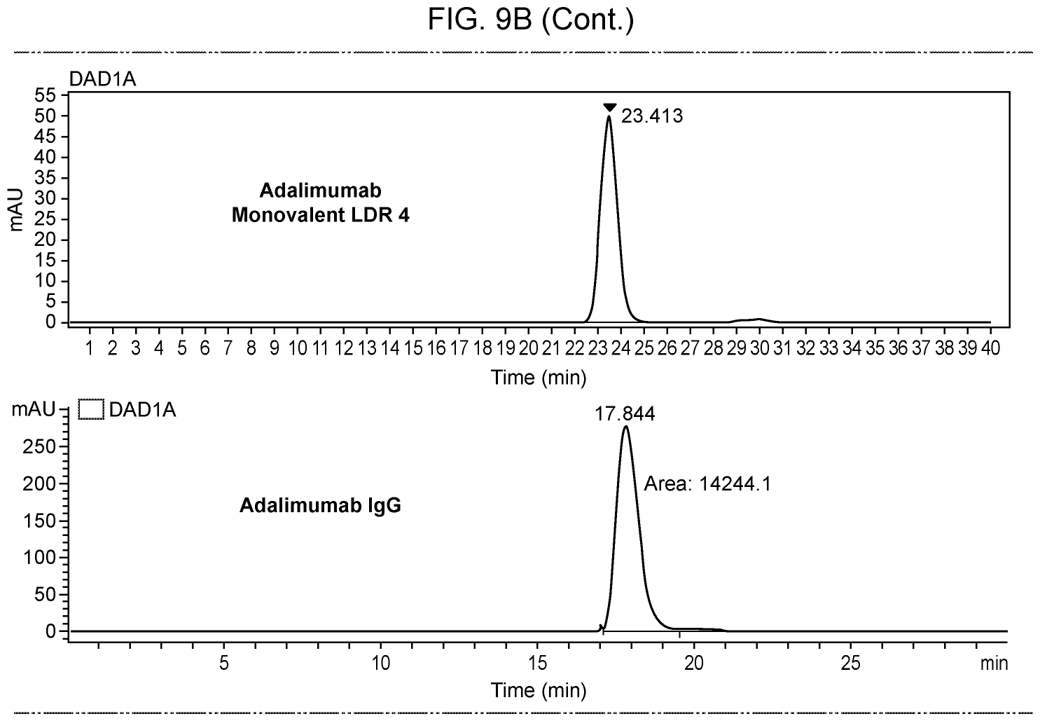

As examples of Siglec-Ligand conjugates used in the sample studies, purity data for adalimumab and adalimumab-Siglec Ligand conjugates are shown in FIG. 9. FIG. 9 depicts example purity and physicochemical characterization data for Adalimumab hIgG1-Siglec Ligand conjugates. Adalimumab conjugates vary in the structure of the Siglec Linker used and the Ligand/Linker-to-Drug Ratio (“LDR”) after conjugation.

FIG. 9A shows capillary gel electrophoresis data for adalimumab conjugates. Owing to the use of random lysine conjugation with the PFP Siglec-Linker compounds, both LC/MS analysis and cGE (capillary gel electrophoresis) analysis reveal heterogeneity in the LDR; as expected each conjugate is an ensemble of species with differing defined LDRs. cGE shows slower mobility and banding consistent with this heterogeneity. LDRs are determined by LC/MS analysis and defined as the weighted average of individual LDR species.

All conjugates were purified to homogeneity for oligomeric species, with the intended oligomeric structure (e.g., monomer, dimer, trimer) being purified by preparative size exclusion chromatography. FIG. 9B shows example purity data for parental adalimumab IgG and adalimumab-Siglec Ligand conjugates, as measured by analytical size exclusion. All parental protein preps and conjugate preparations were >99% pure by analytical SEC.

### Example 8: Suppression of B Cell Receptor-Mediated Activation of Mouse Primary B Cells by Siglec Ligand-Anti-IgD Antibody Conjugates

### Purpose and Introduction

The purpose of this experiment was to test for suppressive effects on mouse B cell activation of B cell receptor (BCR) agonist IgG-Siglec Ligand conjugates.

The platform technology described rests on the premise that activation of B cells through their clonotypic B cell receptor can be suppressed through physical recruitment of the CD22/Siglec-2 inhibitory coreceptor to co-engaged B cell receptor. Without wishing to be bound by theory, CD22 recruited to the B cell receptor is phosphorylated on its ITIM cytoplasmic motif tyrosines by virtue of its proximity to the high local protein kinase activity at the B cell receptor. Phosphorylated CD22 then recruits phosphatases, such as SHP-1 and SHP-2, to the cell surface, in proximity of the B cell activation complex. Such elevated local phosphatase activity dephosphorylates components of the B cell activation complex necessary for B cell activation, thus shutting down responses to B cell receptor engagement. Under normal circumstances, the Siglec-2 immunoinhibitory mechanism acts as a check on aberrant B cell activation, safeguarding against autoreactive antibody production, hyperinflammation, and autoimmunity. The described platform technology exploits this natural phenomenon to cloak foreign proteins as self, dampening B cell activation only on naïve B cell clones that are specific for the given foreign protein and thus blocking immunoglobulin production against the foreign protein, while leaving B cell responses to other antigens intact.

The high diversity of primary B cell populations, and high diversity of B cell receptor sequences and clones (as high as 10¹² per human), presents a challenge for studying BCR agonism in vitro with a single, well-defined BCR antigen. For this reason, pan-BCR activators, such as anti-IgD or anti-IgM antibodies, that can bind, crosslink, and activate the BCR—regardless of B cell/BCR clonality—are used to evaluate BCR activation in vitro. In the experiments described in this example, an anti-mouse IgD monoclonal antibody is used either in parental IgG form or as IgG-Siglec-Ligand conjugates to study the effects of Siglec-2-B cell receptor co-engagement on B cell activation.

To control for impacts of anti-IgD conjugation on BCR binding potency, competition binding assays were used to assess binding activity of Siglec Ligand conjugates and ensure that apparent suppressive effects were due to Siglec-2×BCR co-engagement and not a general damaging of anti-IgD for receptor binding.

### Materials & Methods—In Vitro Murine B-Cell Activation and Surface IgD Competition Assays

Anti-IgD and Anti-IgD-Siglec Ligand test articles were prepared as described in Example 7. Splenocytes from C57BL/6 mice were harvested into single cell suspension, subjected to red cell lysis using ACK buffer, and plated at a concentration of 200,000 cells per well in round bottom 96 well plates in complete RPMI media. Cells were stimulated for 3 hours by the addition of increasing concentration of anti-mouse IgD or Siglec ligand-conjugated anti-mouse IgD. B cell activation was assessed by flow cytometry.

To measure B cell activation following the described stimulation, cells were washed twice by spinning cells at 1200 rpm for 5 minutes and rinsing with PBS. Cells were then resuspended in staining buffer (1% bovine serum albumin/0.1% sodium azide/1×phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were then incubated in the dark for an additional 30 minutes at room temperature. Cells were then washed three times with staining buffer and then analyzed on ZE5 (BioRad). Data analysis was performed using FlowJo (v10.8.0) software.

Competition binding analysis of Siglec ligand-conjugated anti-mouse IgD and anti-mouse IgD binding was performed on splenocytes that were prepared as described above. For this assay, cells were seeded at a concentration of 200,000 cells per well in round bottom 96 well plates in complete RPMI media. Cells were then incubated with mouse Fc-block (BD Biosciences) for five minutes. Following this incubation period, anti-mouse-IgD-AlexaFluor647, at a fixed concentration of 0.14 nM, was added to the cells along with RPMI alone or an increasing titration of non-fluorescently labeled Siglec ligand-conjugated anti-mouse IgD, as well as anti-CD19 and anti-CD45. Cells were incubated at 4° C. for 30 minutes in the dark. Afterwards, cells were washed twice by centrifugation with staining buffer (1% bovine serum albumin/0.1% sodium azide/1×phosphate buffered saline) and antibody binding was analyzed by flow cytometry (ZE5, BioRad) through determination of the mean fluorescence intensity (MFI) of at least 10,000 cells.

### Results and Conclusions

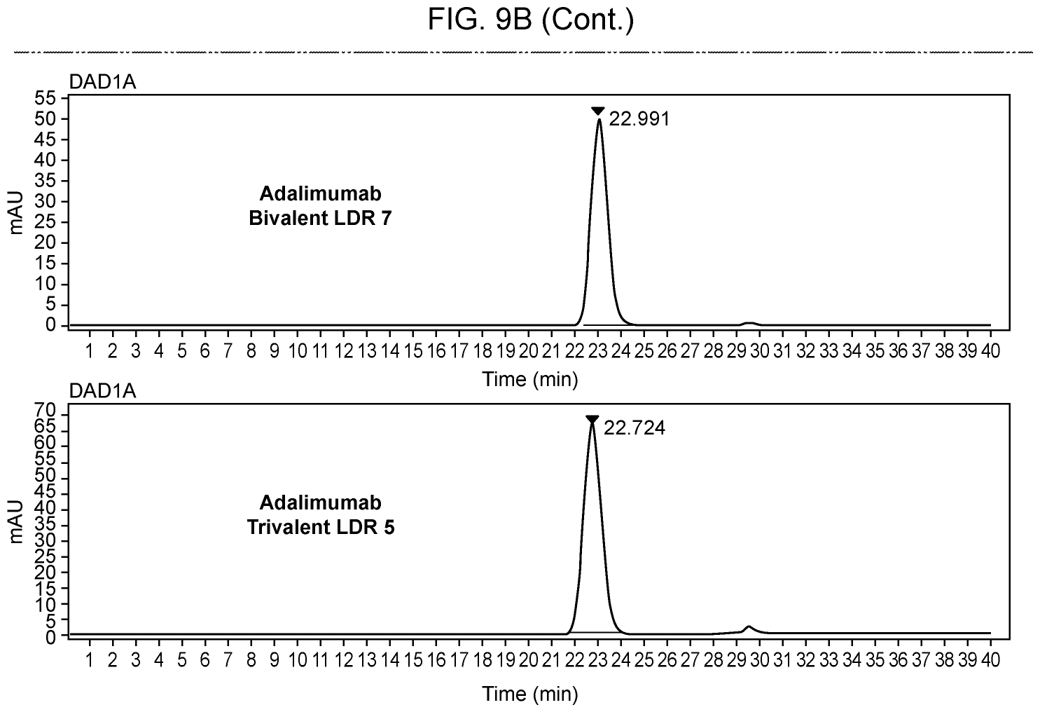

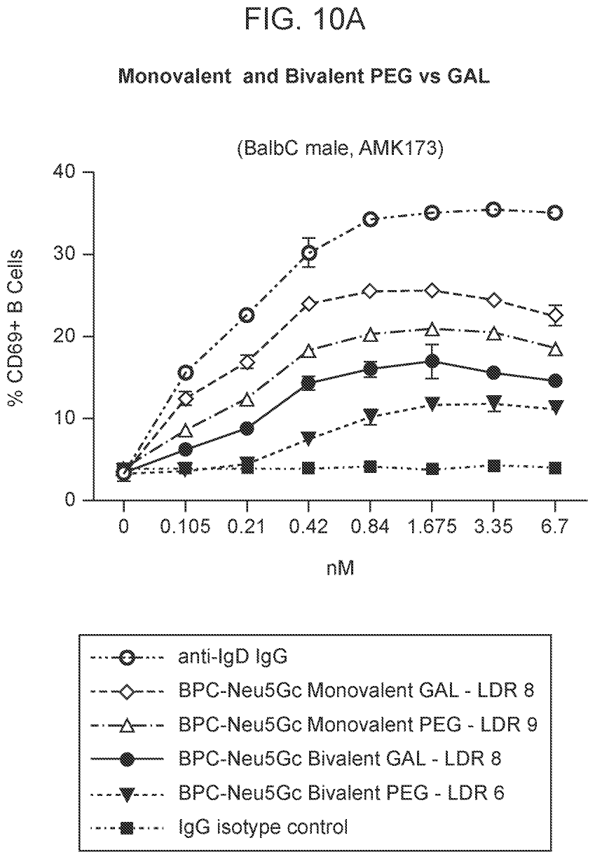

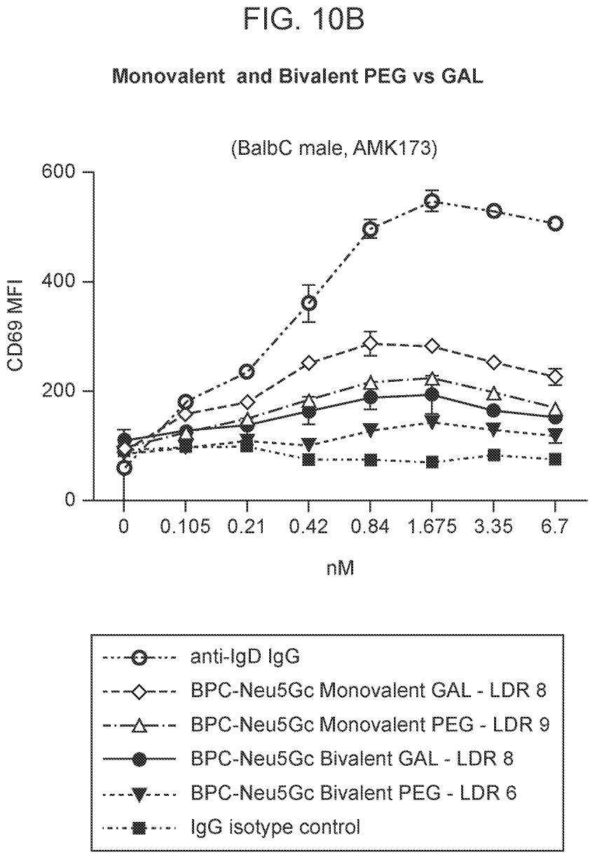

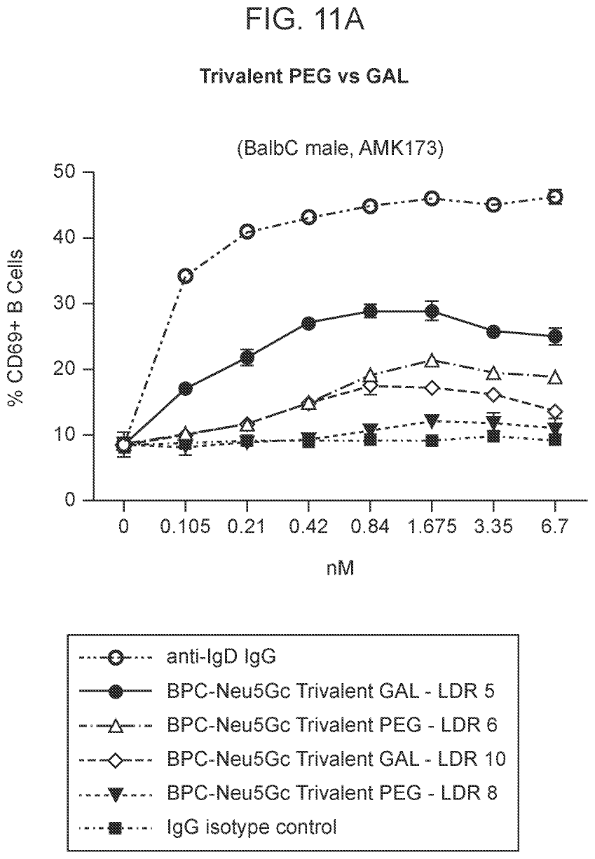

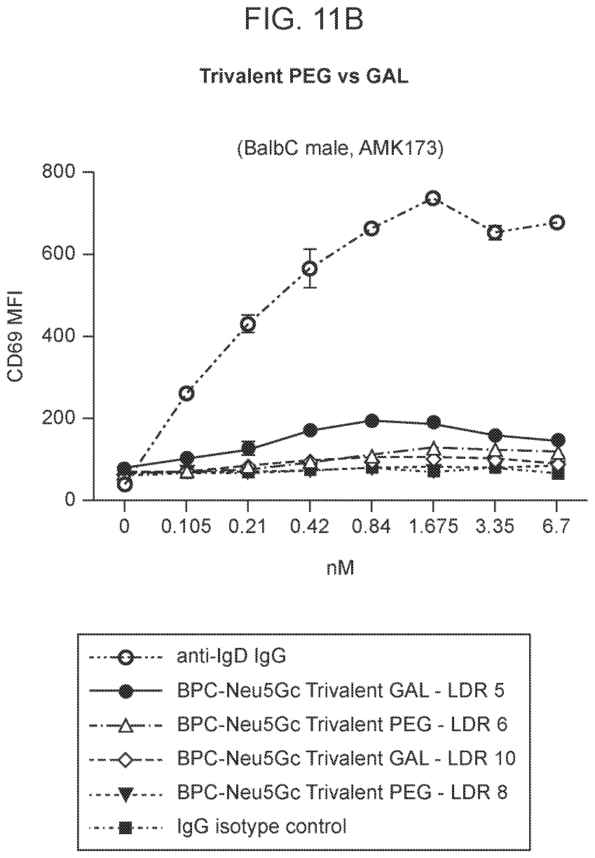

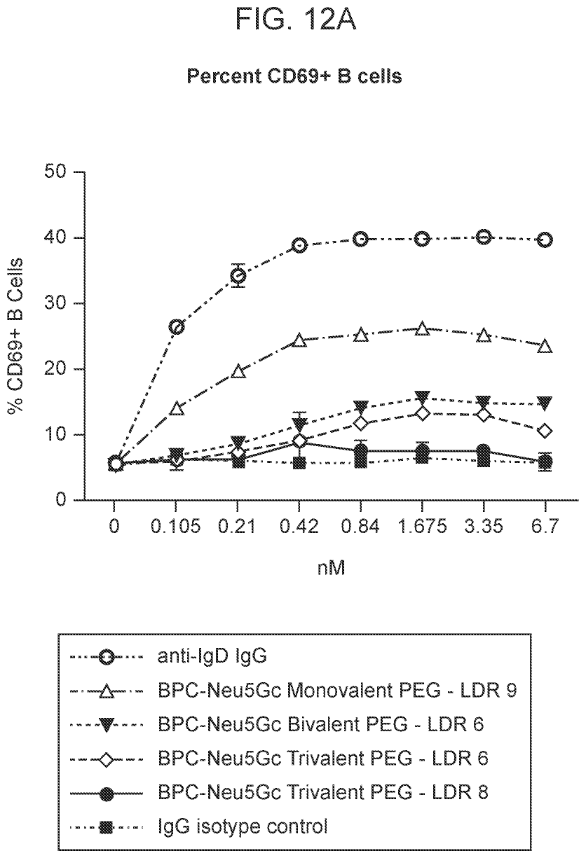

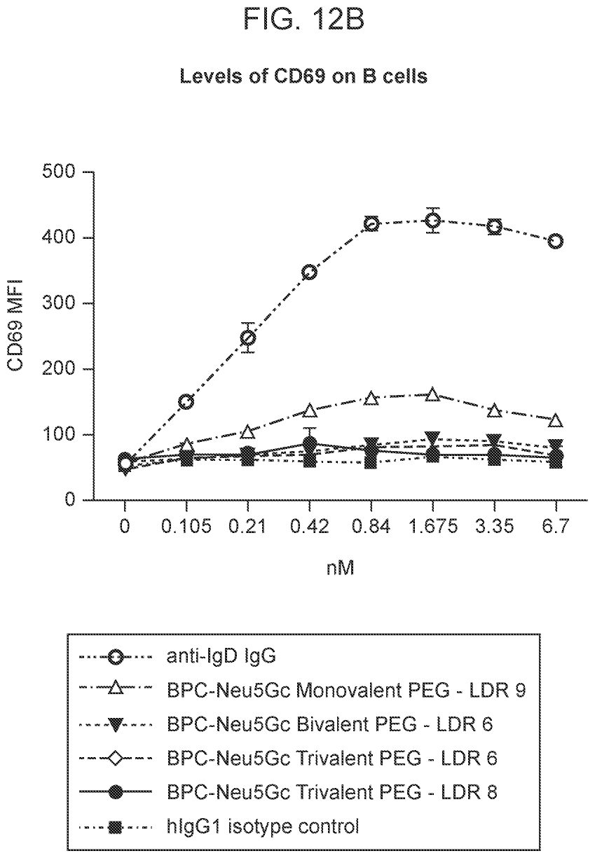

FIG. 10, FIG. 11, and FIG. 12 all depict in vitro B cell activation assays where mouse primary B cells are treated with either a B cell receptor-agonizing anti-IgD antibody or B cell receptor-agonizing anti-IgD-Siglec Ligand conjugates bearing monovalent or bivalent ligand structures. The B cell stimulatory activities of anti-IgD and anti-IgD-Siglec Ligand conjugates are compared in dose titration experiments with an activation readout of CD69 upregulation. CD69 levels on the different treatment groups are evaluated through the percentage of cells that are CD69-positive (FIG. 10A, 11A, 12A) or the CD69 mean fluorescence intensity (MFI) (FIG. 10B, 11B, 12B). Siglec ligands in the tested conjugates vary in linker structure (“PEG” or “Gal”) and valency (“Monovalent” or “Bivalent”), and conjugates vary also in the Ligand/Linker-to-Drug Ratio (“LDR”), or the average number of Siglec Ligand structures per drug molecule. Where parental anti-IgD IgG induces a strong, concentration-dependent increase in % CD69-positive cells and in CD69 MFI, Siglec Ligand-anti-IgD conjugates show suppressed activation. The degree of suppression increases with valency, and is also stronger for PEG-based linkers than for GAL-based linkers. In general, higher valency leads to greater suppression of B cell activation, with Trivalent>Bivalent>Monovalent Siglec Ligand structures for extent of suppression. In certain cases, there is complete suppression of B cell activation (e.g., BPC-Neu5Gc Trivalent PEG-LDR 8).

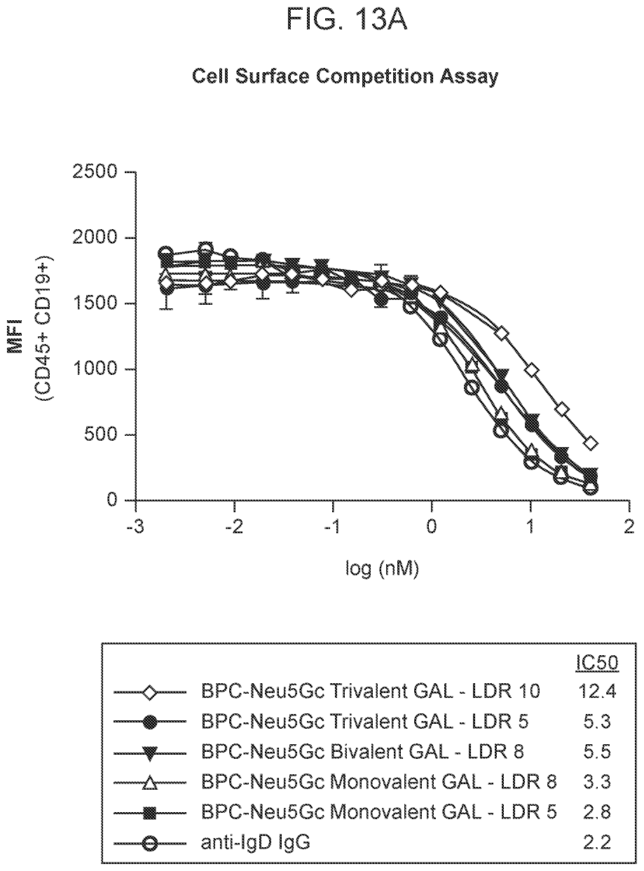

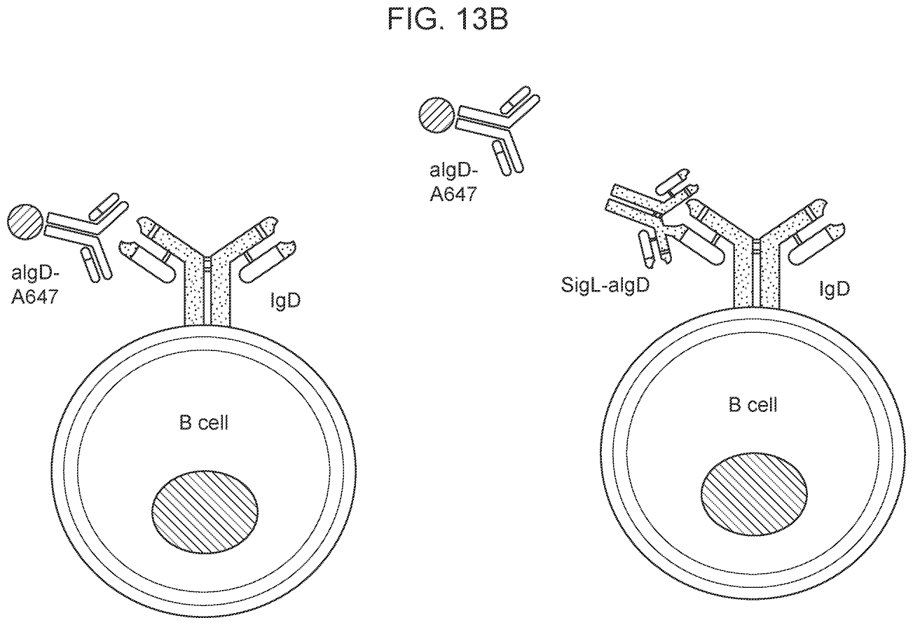

FIG. 13 depicts the evaluation of Siglec Ligand-anti-IgD antibody conjugate binding to mouse primary B cells, in comparison with the parental, unconjugated anti-IgD antibody. Binding is evaluated by fluorescence cytometry, in a competition assay format with Alexa-647-labeled anti-IgD antibody. FIG. 13A depicts dose-response results for concentration-dependent inhibition of fluorescently-labeled anti-IgD binding to IgD⁺ B cells by unlabeled anti-IgD-Siglec Ligand conjugates and unconjugated, unlabeled anti-IgD antibody. The binding IC50, in nanomolar, for each unlabeled test article is indicated. FIG. 13B is a schematic for the binding assay system. The data show no effect or modest effect on binding IC50s for Siglec Ligand conjugates, indicating that the B cell activation observed in FIG. 10, FIG. 11, and FIG. 12 cannot be explained through damage to inherent B cell receptor properties of the conjugates.

### Example 9: Suppression of B Cell Receptor-Mediated Activation of Mouse Primary B Cells by Siglec Ligand-Anti-IgD Antibody Conjugates is Strongly CD22-Dependent

### Purpose

The purpose of these experiments was to evaluate the CD22-dependence of the suppression of B cell receptor-mediated B cell activation by anti-IgD-Siglec Ligand conjugates. This example follows on from the experiments described in Example 8, using pan-B cell receptor agonism in a pool of B cell clones to study the effects of CD22-B cell receptor co-engagement on B cell activation. In this example, primary B cells from wild-type mice were used to evaluate the dependence of BCR suppression on CD22. As in Example 8, competitive BCR binding analysis was used to control for damaging effects of protein lysine conjugation to the BCR binding activity of anti-IgD-Siglec Ligand conjugates.

### Materials and Methods

Anti-IgD and Anti-IgD-Siglec Ligand test articles were prepared as described in Example 7. Splenocytes from C57BL/6 mice were harvested into single cell suspension, subjected to red cell lysis using ACK buffer, and plated at a concentration of 200,000 cells per well in round bottom 96 well plates in complete RPMI media. Cells were stimulated for 3 hours by the addition of increasing concentration of anti-mouse IgD or Siglec ligand-conjugated anti-mouse IgD. B cell activation was then assessed by flow cytometry.

To measure B cell activation following the described stimulation, cells were washed twice by spinning cells at 1200 rpm for 5 minutes and rinsing with PBS. Cells were then resuspended in staining buffer (1% bovine serum albumin/0.1% sodium azide/1×phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were then incubated in the dark for an additional 30 minutes at room temperature. Cells were then washed three times with staining buffer and then analyzed on ZE5 (BioRad). Data analysis was performed using FlowJo (v10.8.0) software.

Competition of Siglec ligand-conjugated anti-mouse IgD and anti-mouse IgD binding was performed on splenocytes that were prepared as described above. For this assay cells were seeded at a concentration of 200,000 cells per well in round bottom 96 well plates in complete RPMI media. Cells were then incubated with mouse Fc-block (BD Biosciences) for five minutes. Following this incubation period, anti-mouse-IgD-AlexaFluor647 at a fixed concentration of 0.14 nM was added to the cells along with RPMI alone or an increasing titration of non-fluorescently labeled Siglec ligand-conjugated anti-mouse IgD, as well as anti-CD19 and anti-CD45 Cells were incubated at 4 C for 30 minutes in the dark. Afterwards cells were washed twice by centrifugation with staining buffer (1% bovine serum albumin/0.1% sodium azide/1×phosphate buffered saline) and antibody binding was analyzed by flow cytometry (ZE5, BioRad) by determination of the mean fluorescence intensity (MFI) of at least 10,000 cells.

### Results and Conclusions

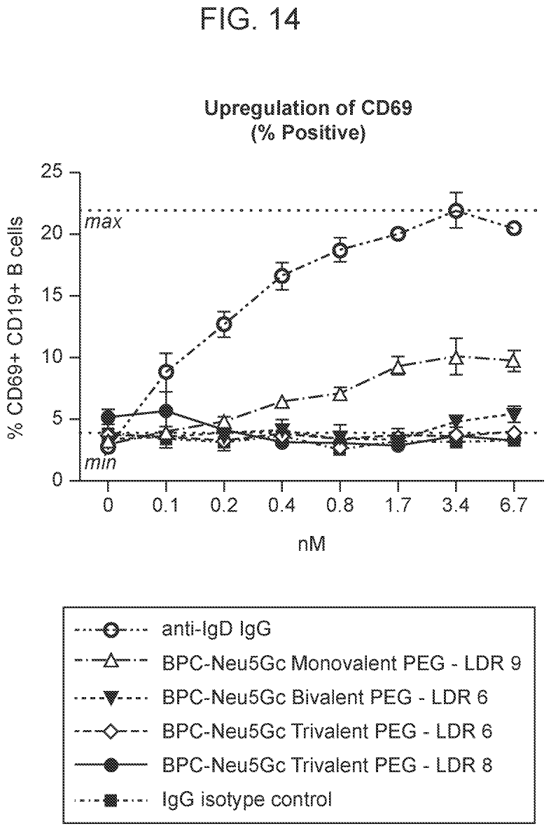

FIG. 14 depicts an in vitro B cell activation assay where mouse primary B cells are treated with either a B cell receptor agonizing anti-IgD antibody or anti-IgD-Siglec Ligand conjugates, where the same test articles are used to treat B cells from wild-type mice. The conjugate Siglec Ligands (“BPC-Neu5Gc Monovalent PEG-LDR 9”, “BPC-Neu5Gc Bivalent PEG-LDR 6”, “BPC-Neu5Gc Trivalent PEG-LDR 6”, and “BPC-Neu5Gc Trivalent PEG-LDR 8”) are potentiated for Siglec-2 binding. The B cell stimulatory activities of anti-IgD and anti-IgD-Siglec Ligand conjugates are compared in dose titration experiments with an activation readout of CD69 upregulation. CD69 levels on the different treatment groups are evaluated through the percentage of cells that are CD69-positive. Siglec ligands in the tested conjugates contain PEG-based linker structures (“PEG”) and vary in valency (“Monovalent”, “Bivalent”, and “Trivalent”). Conjugates vary also in the Ligand/Linker-to-Drug Ratio (“LDR”), or the average number of Siglec Ligand structures per drug molecule. As shown previously in Example 8 and in FIG. 10, FIG. 11, and FIG. 12, wild-type B cell activation is strongly suppressed with Siglec Ligand-bearing anti-IgD conjugates. In contrast, B cells from CD22 knockout (“CD22 KO”) mice can show full B cell activation activity for Siglec Ligand-conjugates, with no effect or only modest effects on activation potency. Importantly, these results indicate that the suppressive effects of Siglec-2 Ligand conjugates is mediated by Siglec-2/CD22, as expected.

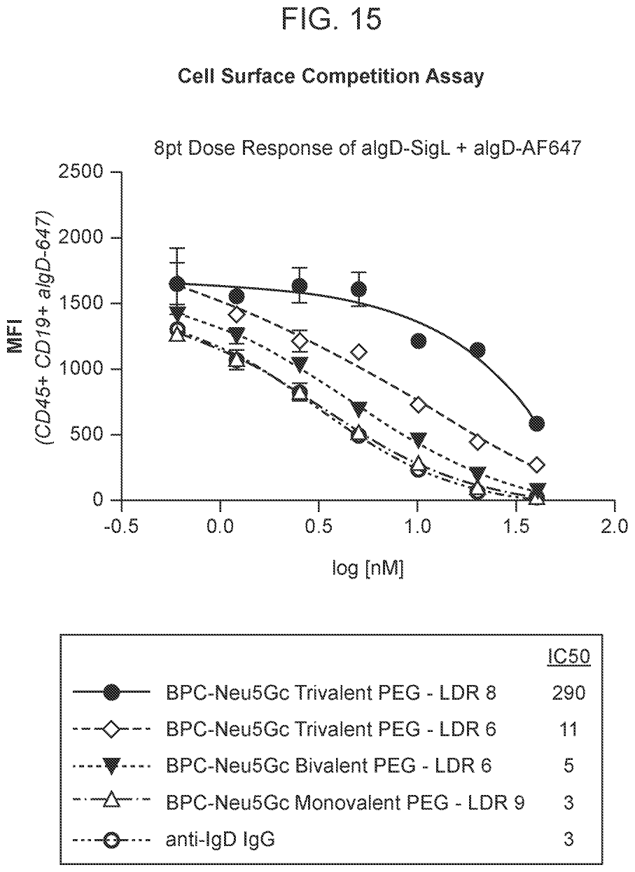

As tested previously in Example 8 (FIG. 13), the BCR binding activities of Siglec Ligand-anti-IgD conjugates and parental IgD were assessed in a cytometry assay (FIG. 15), with identical preparations to those tested above in FIG. 14 for BCR activation. Competition binding analysis reveals that all conjugate preps except the highest LDR Trivalent conjugate (BPC-Neu5Gc Trivalent PEG-LDR 8) are fully active for BCR binding. These results corroborate CD22 KO activation experiment result, where suppression of B cell activation in anti-IgD conjugates could not be explained due to inactivation of BCR.

### Example 10: Suppression of B Cell Receptor-Mediated Activation of Mouse Primary B Cells by Siglec Ligand-Anti-IgD Antibody Conjugates Requires Potentiation of Siglec-2/CD22 Binding

### Purpose

The purpose of the experiments described here was to evaluate the importance of potentiated Siglec-2 binding for suppression of B cell receptor-mediated B cell activation. This example follows on from the experiments described in Examples 8 and 9, using pan-B cell receptor agonism in a pool of B cell clones to study the effects of CD22-B cell receptor co-engagement on mouse B cell activation. In this example, the Siglec-2 ligands presented on the prepared conjugates were varied in their potency for Siglec-2 binding, using either potentiated Siglec-2 ligand conjugates (BPC-Neu5Gc-based) or Siglec-2 ligands based on native Neuraminic acid structures (Neu5Gc). The Siglec Ligands used here were previously evaluated in Example 4 and verified for CD22 binding potency. Comparison of conjugates bearing potentiated and non-potentiated Siglec-2 enabled evaluation of the importance of Siglec-2 affinity for the B cell receptor-suppressive effects of the described platform technology.

### Materials and Methods

Anti-IgD and Anti-IgD-Siglec Ligand test articles were prepared as described in Example 7. Splenocytes from C57BL/6 mice were harvested into single cell suspension, subjected to red cell lysis using ACK buffer, and plated at a concentration of 200,000 cells per well in round bottom 96 well plates in complete RPMI media. Cells were stimulated for 3 hours by the addition of increasing concentration of anti-mouse IgD or Siglec ligand-conjugated anti-mouse IgD. B cell activation was assessed by flow cytometry.

To measure B cell activation following the described stimulation, cells were washed twice by spinning cells at 1200 rpm for 5 minutes and rinsing with PBS. Cells were then resuspended in staining buffer (1% bovine serum albumin/0.1% sodium azide/1×phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were then incubated in the dark for an additional 30 minutes at room temperature. Cells were then washed three times with staining buffer and then analyzed on ZE5 (BioRad). Data analysis was performed using FlowJo (v10.8.0) software.

Competition of Siglec ligand-conjugated anti-mouse IgD and anti-mouse IgD binding was performed on splenocytes that were prepared as described above. For this assay cells were seeded at a concentration of 200,000 cells per well in round bottom 96 well plates in complete RPMI media. Cells were then incubated with mouse Fc-block (BD Biosciences) for five minutes. Following this incubation period, anti-mouse-IgD-AlexaFluor647 at a fixed concentration of 0.14 nM was added to the cells along with RPMI alone or an increasing titration of non-fluorescently labeled Siglec ligand-conjugated anti-mouse IgD, as well as anti-CD19 and anti-CD45 Cells were incubated at 4 C for 30 minutes in the dark. Afterwards cells were washed twice by centrifugation with staining buffer (1% bovine serum albumin/0.1% sodium azide/1×phosphate buffered saline) and antibody binding was analyzed by flow cytometry (ZE5, BioRad) by determination of the mean fluorescence intensity (MFI) of at least 10,000 cells.

### Results and Conclusions

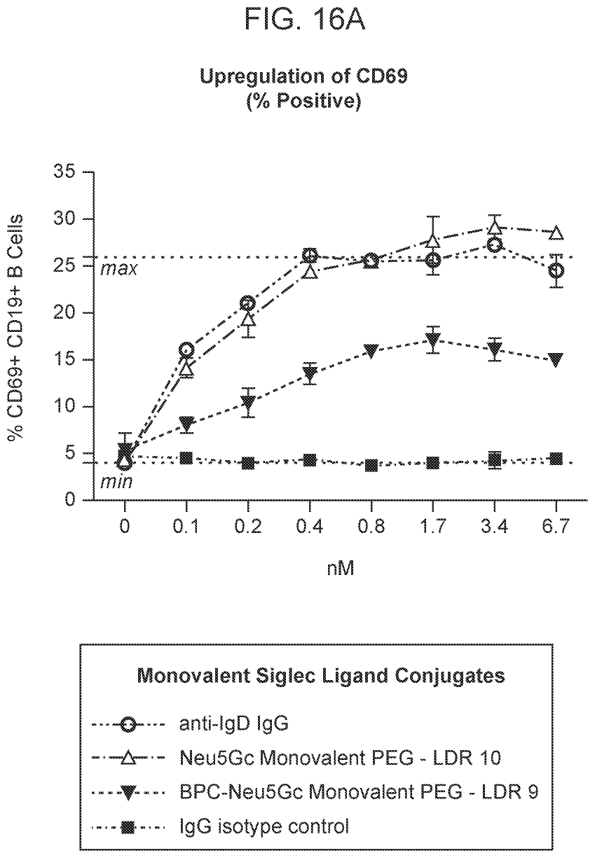

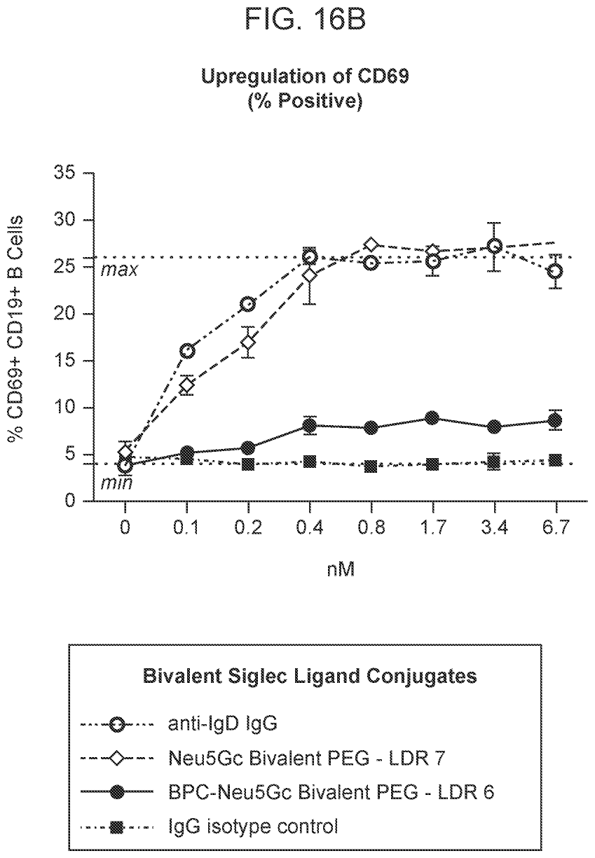

FIG. 16 depicts an in vitro B cell activation assay where mouse primary B cells are treated with either a B cell receptor agonizing anti-IgD antibody or anti-IgD-Siglec Ligand conjugates, where the Siglec Ligands are potentiated (“BPC-Neu5Gc Monovalent PEG-LDR 9” and “BPC-Neu5Gc Bivalent PEG-LDR 6”) or unpotentiated (“Neu5Gc Monovalent PEG-LDR 10” and “Neu5Gc Bivalent PEG-LDR 7”) for Siglec-2 binding. The B cell stimulatory activities of anti-IgD and anti-IgD-Siglec Ligand conjugates are compared in dose titration experiments with an activation readout of CD69 upregulation. CD69 levels on the different treatment groups are evaluated through the percentage of cells that are CD69-positive. Dose response curves are shown for monovalent (FIG. 16A) and bivalent (FIG. 16B) Siglec Ligand conjugates. Conjugates vary also in the Ligand/Linker-to-Drug Ratio (“LDR”), or the average number of Siglec Ligand structures per drug molecule. Where potentiated Siglec Ligand conjugates show strong suppression of B cell activation, conjugates bearing unpotentiated Neu5Gc Siglec ligands show a complete absence of suppression of B cell activation. Importantly, this example reveals that native neuraminic affinities for CD22 are insufficient to mediate the BCR suppressive effect of Siglec Ligand conjugate. These results also confirm that BCR activation suppression effects are not a simple and trivial consequence of damage to anti-IgD antibody through conjugation; once again, conjugated anti-IgD molecules are fully active for BCR activation either where Siglec-2 binding is too weak or where B cells lack CD22.

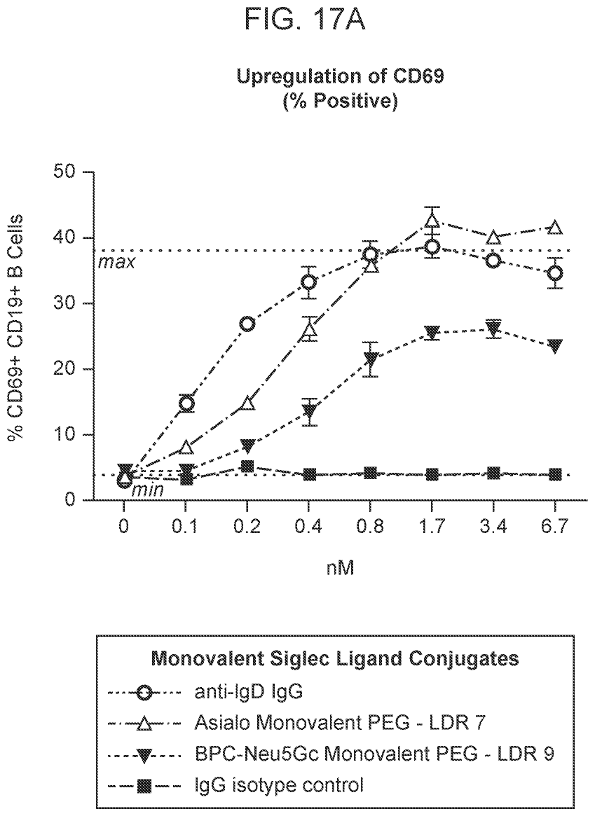

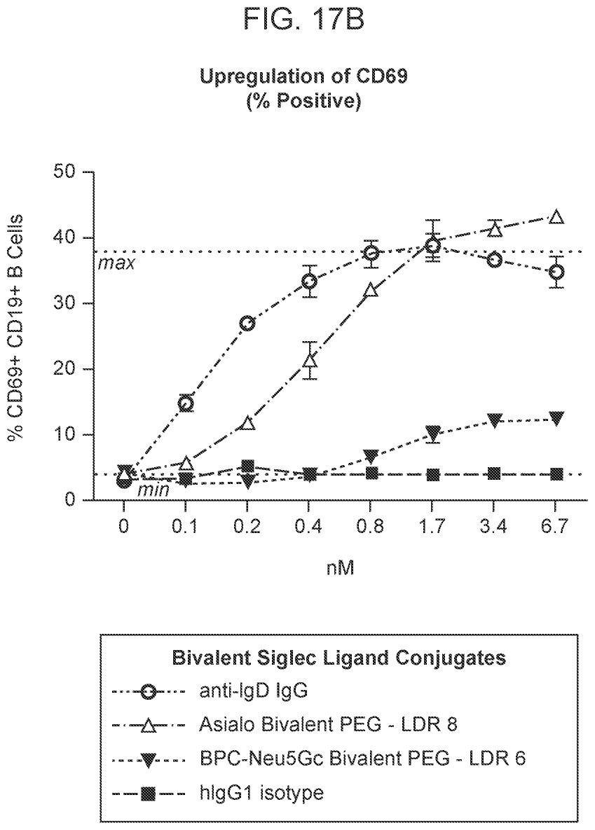

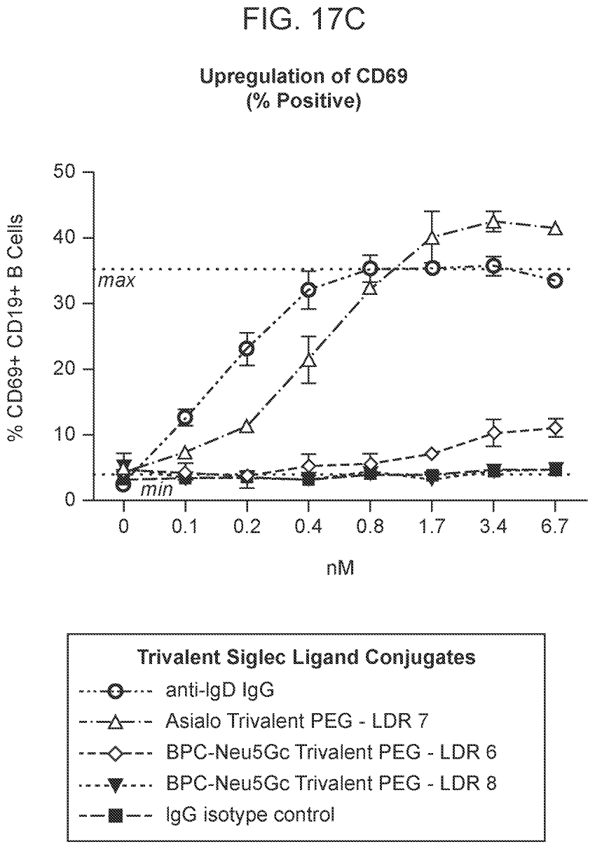

FIG. 17 depicts a separate in vitro primary mouse B cell activation assay testing for the importance of CD22 engagement for Siglec Ligand-conjugate-mediated suppression of B cell receptor activation. As in FIG. 16 and Examples 8 and 9, mouse primary B cells were treated with either a B cell receptor agonizing anti-IgD antibody or various anti-IgD-Siglec Ligand conjugates. In this case, conjugates carried either potentiated Siglec Linkers (“BPC-Neu5Gc Monovalent PEG-LDR 9”, “BPC-Neu5Gc Bivalent PEG-LDR 6”, “BPC-Neu5Gc Trivalent PEG-LDR 6”, and “BPC-Neu5Gc Trivalent PEG-LDR 8”), or negative control, asialo linkers lacking Siglec binding determinants (“Asialo Monovalent PEG-LDR 7”, “Asialo Bivalent PEG-LDR 8”, and “Asialo Trivalent PEG-LDR 7”). As with conjugates bearing weak affinity Siglec-2 ligands, conjugates bearing linkers that are absent for Siglec-2 binding activity are completely active for BCR and B cell activation.

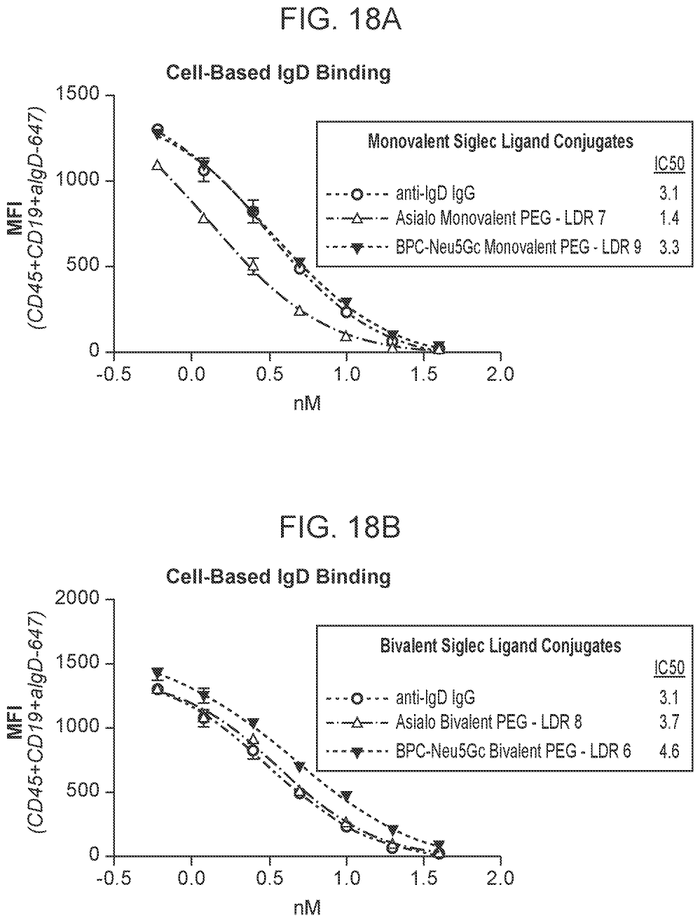

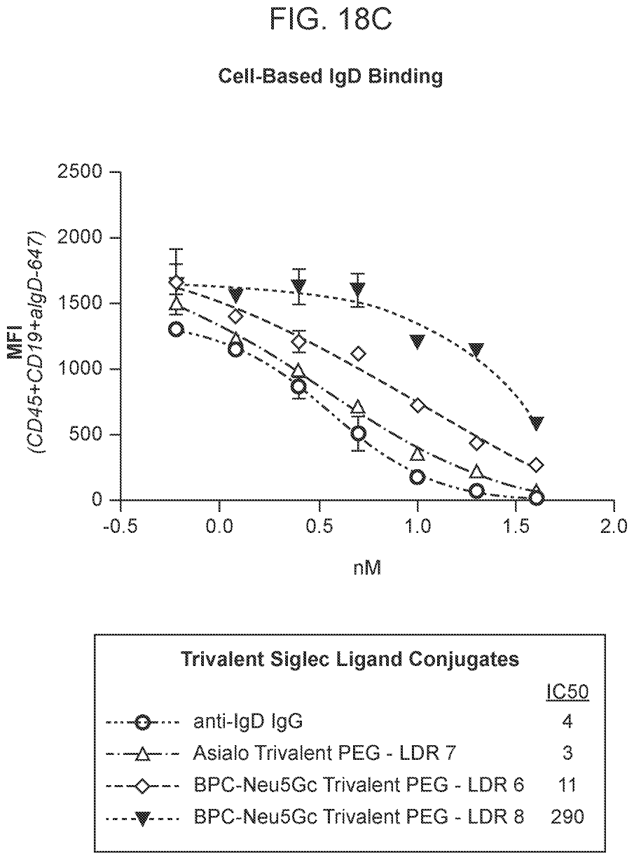

As in Examples 8 and 9, conjugates (potentiated and asialo forms) and parental anti-IgD were evaluated for binding activity in a competition cytometry assay (FIG. 18). The test articles are identical to those used for FIG. 17. Monovalent and bivalent conjugates show identical binding IC50s to parental anti-IgD. For trivalent conjugates, low LDR preparations are unperturbed for binding, while, as in FIG. 15, high LDR Trivalent conjugate shows a weaker binding IC50 in the competition binding assay. Overall, these results corroborate the above example results, where Siglec Ligand anti-IgD conjugates can retain full functional activity for BCR binding while suppressed for BCR activation through Siglec-2/CD22 co-engagement activity.

### Example 11: Suppression of B Cell Receptor-Mediated Activation of Mouse Primary B Cells by Siglec Ligand-Anti-IgD Antibody Conjugates Requires Engagement of BCR and CD22/Siglec-2 in Cis

### Purpose and Introduction

The purpose of the experiments described here is to evaluate the importance of cis co-engagement by the same molecules on CD22/Siglec-2 and the B cell receptor for suppression of B cell activation through the B cell receptor. This example follows on from the experiments described in Examples 8, 9 and 10, using pan-B cell receptor agonism in a pool of B cell clones to study the effects of CD22-B cell receptor co-engagement on B cell activation. In this example, the Siglec-2 ligands (potentiated, BPC-Neu5Gc-based) were either present in cis on the anti-IgD (as in the above experiments) or in trans on a separate, negative control antibody (adalimumab anti-TNFα) that does not engage the B cell receptor. In the latter case, adalimumab-Siglec Ligand was co-adminstered to splenocytes with a fixed concentration of unmodified anti-IgD BCR agonist antibody.

A second experiment evaluated the outcome for B cell activation in cases where there were mixtures of BCR agonist antibody and Siglec Ligand-BCR agonist antibody conjugate. Mixtures of anti-IgD IgG and Siglec Ligand-anti-IgD conjugate were evaluated for suppression of B cell activation, where BPC-Neu5Gc Bivalent PEG LDR6-anti-IgD was added at varying concentrations in the presence or absence of 2 nM anti-IgD agonist antibody.

### Materials and Methods

Anti-IgD and Anti-IgD-Siglec Ligand test articles were prepared as described in Example 7.

Assays to determine if the SigL-mediated suppression of B cell activation occurs in cis or trans were performed on splenocytes from C57BL/6 mice that were prepared as described above. For this assay cells were seeded at a concentration of 200,000 cells per well in round bottom 96 well plates in complete RPMI media. Cells were incubated with mouse Fc-block (BD Biosciences) for five minutes. Following this incubation period, an increasing concentration of either human IgG1 isotype, anti-mouse IgD, Siglec Ligand-conjugated anti-mouse IgD, or Siglec Ligand-conjugated adalimumab was added to the cells. In a separate set of conditions, a fixed concentration of 2 nM anti-mouse IgD was added to cells along with an increasing concentration of SigL-conjugated adalimumab. Cells were stimulated for 3 hours, and then B-cell activation was assessed by flow cytometry.

To measure B cell activation following the described stimulation, cells were washed twice by spinning cells at 1200 rpm for 5 minutes and rinsing with PBS. Cells were then resuspended in staining buffer (1% bovine serum albumin/0.1% sodium azide/1×phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were then incubated in the dark for an additional 30 minutes at room temperature. Cells were then washed three times with staining buffer and then analyzed on ZE5 (BioRad). Data analysis was performed using FlowJo (v10.8.0) software.

### Results and Conclusions

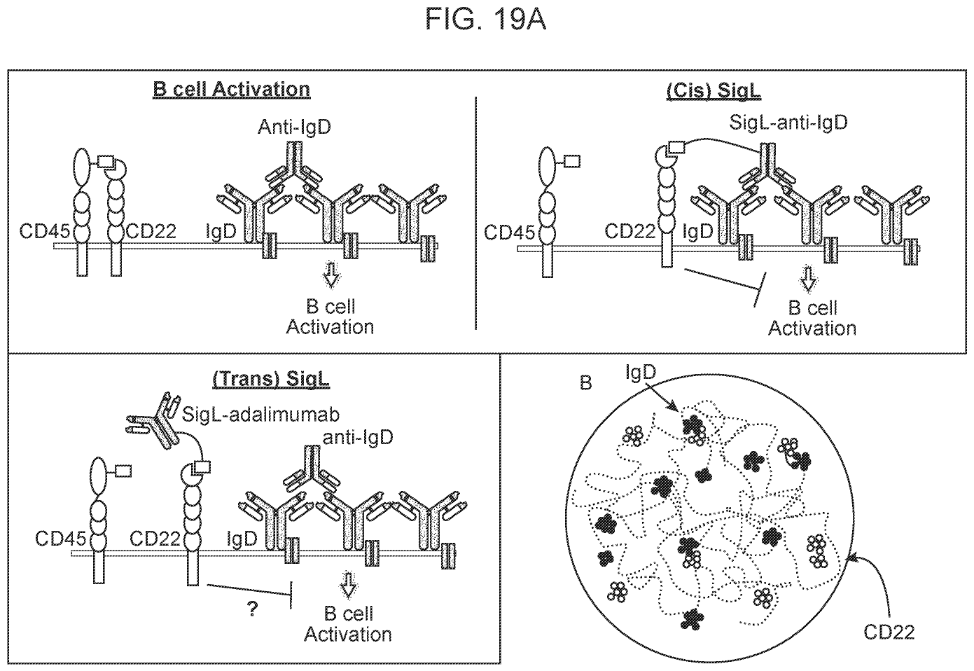

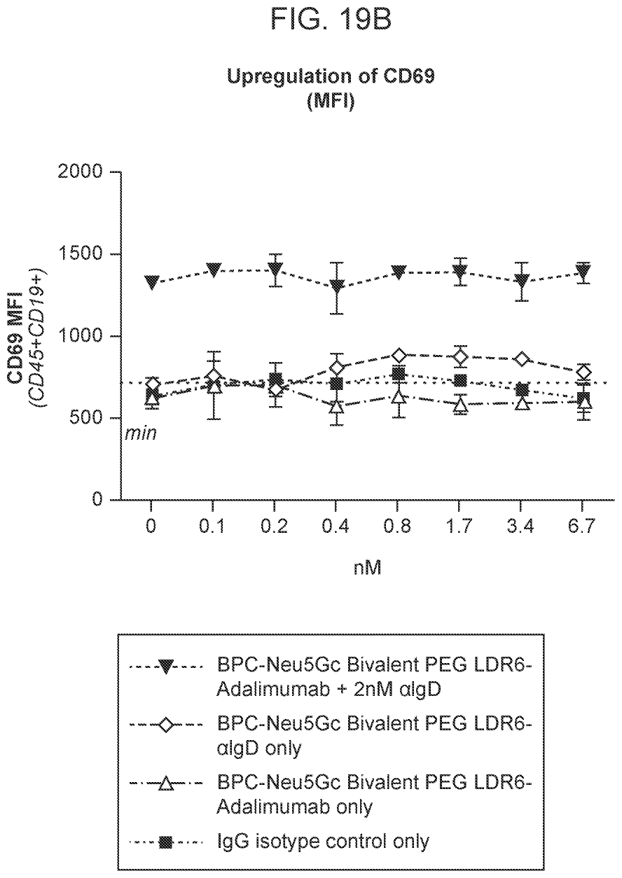

FIG. 19 depicts an in vitro primary mouse B cell activation assay testing for the importance of cis B cell receptor and CD22 co-engagement for suppression of B cell receptor activation. FIG. 19A depicts a model for B cell receptor activation where the anti-IgD BCR agonist and Siglec-2-engaging moieties are presented on the same or separate molecules. The B cell stimulatory activities of anti-IgD, anti-IgD-Siglec Ligand conjugate, or a mixture of 2 nM anti-IgD and varying concentrations of control antibody-Siglec Ligand conjugate were compared in dose titration experiments with an activation readout of CD69 upregulation. CD69 levels on the different treatment groups were evaluated through the CD69 mean fluorescence intensity (MFI) (FIG. 19B). Siglec ligands in the tested conjugates contain PEG-based linker structures (“PEG”) and bear bivalent Siglec Ligand structures. The data show a clear requirement that BCR agonist activity and Siglec-2 binding activity be in cis in the same molecule.

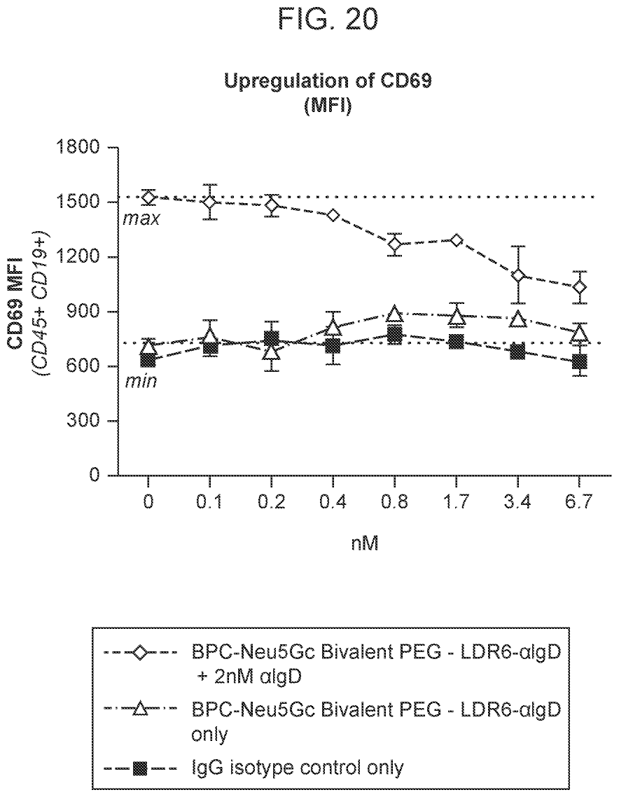

FIG. 20 depicts an in vitro primary mouse B cell activation assay testing for BCR agonism suppression in mixtures of agonistic anti-IgD antibody and non-agonistic Siglec Ligand-anti-IgD conjugate. Siglec Ligand-anti-IgD conjugate is titrated in the presence or absence of 2 nM anti-IgD BCR agonist. The B cell stimulatory activities of anti-IgD, anti-IgD-Siglec Ligand conjugate, or a mixture of 2 nM anti-IgD with varying concentrations of anti-IgD-Siglec Ligand conjugate are compared in dose titration experiments with an activation readout of CD69 upregulation. CD69 levels on the different treatment groups are evaluated through the CD69 mean fluorescence intensity (MFI). The anti-IgD conjugate tested bears a bivalent, Siglec-2-potentiated ligand (MPB-Neu5Ac), and a PEG-based linker, with a Ligand/Linker-to-Drug Ratio (“LDR”) of 6.

The results show that Siglec Ligand conjugate must be titrated roughly iso-stoichiometrically with anti-IgD antibody to inhibit 50% of B cell activation. Thus, neither anti-IgD no Siglec Ligand-anti-IgD conjugate is dominant for BCR outcomes. Both compete equally for effects on B cell activation.

### Example 12: Suppression of B Cell Receptor-Mediated Activation of Human Primary B Cells by Siglec Ligand-Anti-IgM Antibody Conjugates

### Purpose

The purpose of this experiment was to test for suppressive effects on human B cell activation with B cell receptor agonist IgG-Siglec Ligand conjugates. This experiment is analogous to the one described in Example 8, with the focus here on primary human, PBMC-derived B cells, rather than the primary mouse splenocytes used in Examples 8 to 11.

The platform technology described rests on the premise that activation of B cells through their clonotypic B cell receptor can be suppressed through recruitment of the CD22/Siglec-2 inhibitory coreceptor in close proximity to co-engaged B cell receptor. CD22 recruited to the B cell receptor is phosphorylated on its ITIM cytoplasmic motif tyrosines by virtue of its proximity to the high local protein kinase activity at the B cell receptor. Phosphorylated CD22 then recruits phosphatases, such as SHP-1 and SHP-2, to the cell surface, in proximity of the B cell activation complex. Such elevated local phosphatase activity dephosphorylates components of the B cell activation complex necessary for B cell activation, thus shutting down responses to B cell receptor engagement. Under normal circumstances, the Siglec-2 immunoinhibitory mechanism acts as a check on aberrant B cell activation, safeguarding against autoreactive antibody production, hyperinflammation, and autoimmunity. The described platform technology exploits this natural phenomenon to cloak foreign proteins as self, dampening B cell activation only on naïve B cell clones that are specific for the given foreign protein and thus blocking immunoglobulin production for the foreign protein, while leaving B cell responses to other antigens intact.

### Materials and Methods

Anti-IgM and Anti-IgM-Siglec Ligand test articles were prepared as described in Example 7.

Human PBMCs (StemExpress) were plated at a concentration of 200,000 cells per well in round bottom 96 well plates in complete RPMI media. Cells were stimulated for 18 hours by the addition of increasing concentration of anti-human IgM or Siglec ligand-conjugated anti-human IgM. B cell activation was assessed by flow cytometry.

To measure B cell activation following the described stimulation, cells were washed twice by spinning cells at 1200 rpm for 5 minutes and rinsing with PBS. Cells were resuspended in staining buffer (1% bovine serum albumin/0.1% sodium azide/1×phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were incubated in the dark for an additional 30 minutes at room temperature, then washed three times with staining buffer and then analyzed on ZE5 (BioRad). Data analysis was performed using FlowJo (v10.8.0) software.

### Results

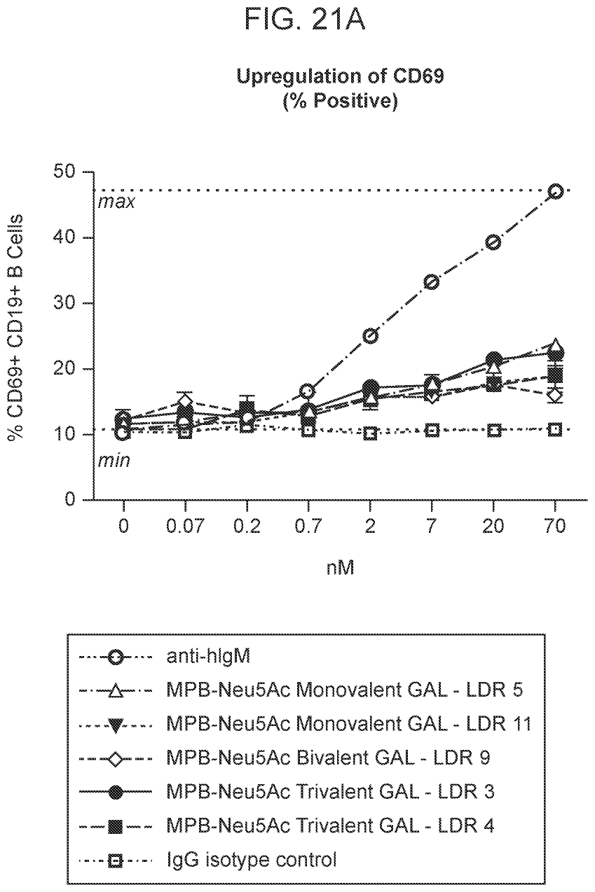

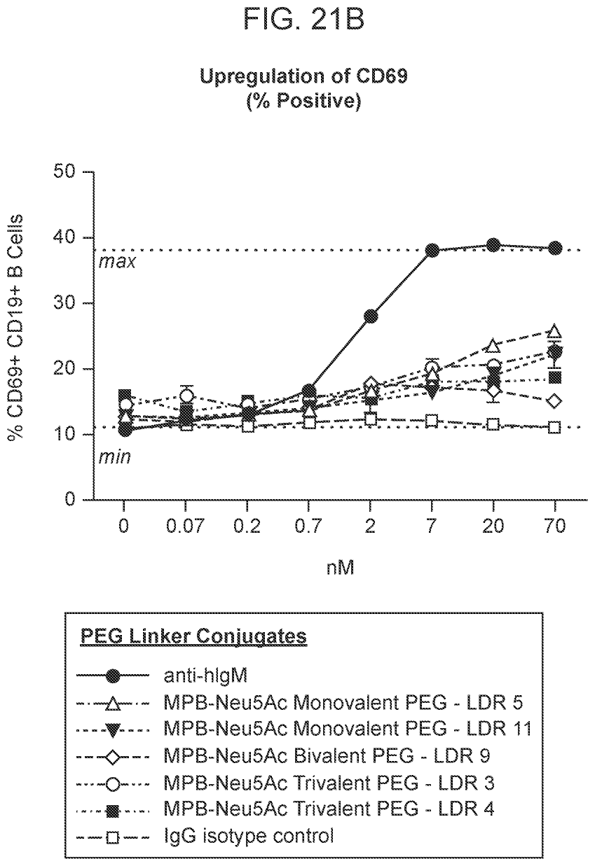

FIG. 21 depicts an in vitro B cell activation assay, using human primary B cells and either a B cell receptor agonizing anti-IgM antibody or Siglec Ligand conjugates with the same anti-IgM antibody. The B cell stimulatory activities of anti-IgM and anti-IgM-Siglec Ligand conjugates are compared in dose titration experiments with an activation readout of CD69 upregulation. CD69 levels on the different treatment groups are evaluated through the percentage of cells that are CD69-positive. Results with conjugates using galactose-based linkers (FIG. 21A) and PEG-based linkers (FIG. 21B) are shown. All conjugates bear Siglec-2-potentiated MPB-Neu5Ac structures, with varying valency and Ligand/Linker-to-Drug Ratio (“LDR”), or the average number of Siglec Ligand structures per drug molecule.

### Conclusions

Where parental anti-IgM IgG induces a strong, concentration-dependent increase in % CD69-positive cells and in CD69 MFI, Siglec Ligand-anti-IgD conjugates show very strongly suppressed activation. The degree of suppression increases with valency, but is generally equivalent between PEG-based and GAL-based linkers. Importantly, these data show translation of the suppressive effect of Siglec-2 Ligand conjugates between a primary human B cell system and those shown in a primary mouse B cell system (Examples 8 to 11).

### Example 13: Suppression of B Cell Receptor-Mediated Activation of Human Primary B Cells by Siglec Ligand-Anti-IgM Antibody Conjugates Requires Potentiation of Siglec-2/CD22 Binding

### Purpose

The purpose of the experiments described here is to evaluate the importance of potentiated Siglec-2 binding for suppression of human B cell receptor-mediated B cell activation. This example follows on from the experiment described in Example 12, using pan-B cell receptor agonism in a pool of human B cell clones to study the effects of CD22-B cell receptor co-engagement on B cell activation. In this example, the Siglec-2 ligands presented on the prepared conjugates were varied in their potency for Siglec-2 binding, using either potentiated Siglec-2 ligand conjugates (BPC-Neu5Gc-based) or Siglec-2 ligands based on native Neuraminic acid structures (Neu5Gc). Comparison of conjugates bearing potentiated and non-potentiated Siglec-2 enables evaluation of the importance of Siglec-2 affinity for the B cell receptor-suppressive effects of the described platform technology.

These experiments with human B cells are analogous to the experiments described in Example 10 for suppression of mouse B cell activation with potentiated and non-potentiated Siglec-2 ligands.

### Materials and Methods

Anti-IgM and Anti-IgM-Siglec Ligand test articles were prepared as described in Example 7.

Human PBMCs (StemExpress) were plated at a concentration of 200,000 cells per well in round bottom 96 well plates in complete RPMI media. Cells were stimulated for 18 hours by the addition of increasing concentration of anti-human IgM or Siglec ligand-conjugated anti-human IgM. B cell activation was assessed by flow cytometry.

To measure B cell activation following the described stimulation, cells were washed twice by spinning cells at 1200 rpm for 5 minutes and rinsing with PBS. Cells were resuspended in staining buffer (1% bovine serum albumin/0.1% sodium azide/1×phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were incubated in the dark for an additional 30 minutes at room temperature, then washed three times with staining buffer and then analyzed on ZE5 (BioRad). Data analysis was performed using FlowJo (v10.8.0) software.

Competition of Siglec ligand-conjugated anti-human IgD and anti-human IgM binding was carried out on human PBMCs from the same donors used in assays described above. For this assay, cells were seeded at a concentration of 200,000 cells per well in round bottom 96 well plates in complete RPMI media. Cells were subsequently incubated with human Fc-block (BD Biosciences) for five minutes. Following this incubation period, 2.4 nM anti-human-IgM-AlexaFluor647 was added to the cells along with RPMI alone or an increasing titration of non-fluorescently labeled Siglec ligand-conjugated anti-human IgM and anti-CD19. Cells were incubated at 4° C. for 30 minutes in the dark. After incubation, cells were washed twice by centrifugation with staining buffer (1% bovine serum albumin/0.1% sodium azide/1×phosphate buffered saline) and antibody binding was analyzed by flow cytometry (ZE5, BioRad) by determination of the mean fluorescence intensity (MFI) of at least 5,000 cells.

### Results and Conclusions

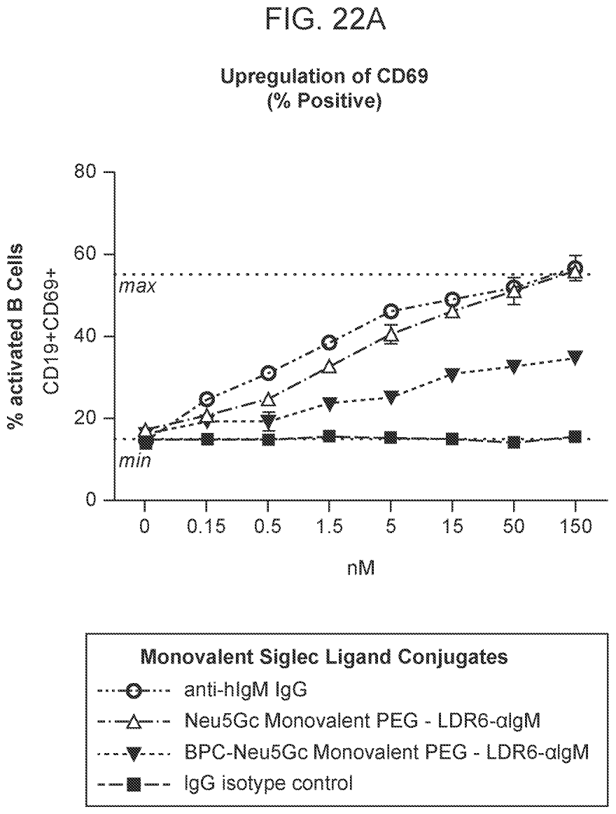

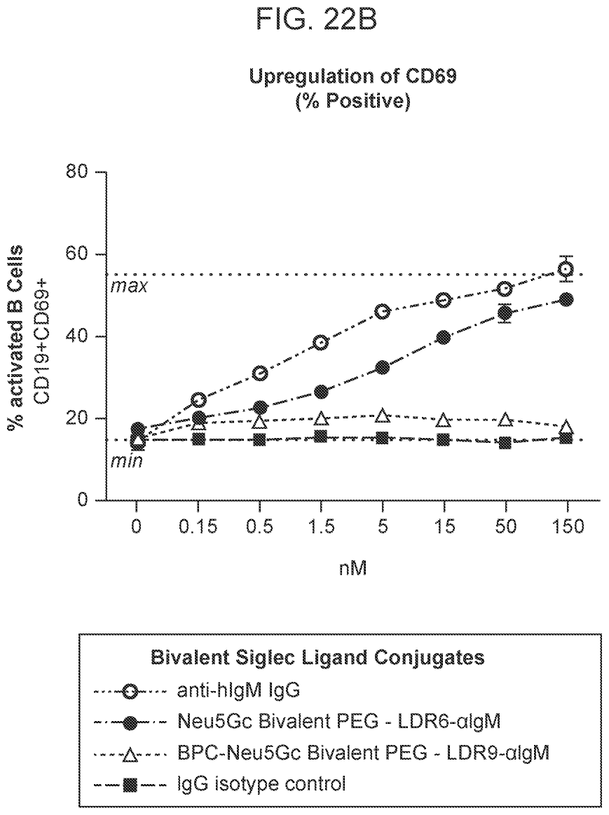

FIG. 22 depicts an in vitro B cell activation assay, using human primary B cells and either a B cell receptor agonizing anti-IgM antibody or Siglec Ligand conjugates with the same anti-IgM antibody. Conjugates bear Siglec Ligand structures that are either potentiated (“BPC-Neu5Gc Monovalent PEG-LDR5-αIgM” and “BPC-Neu5Gc Bivalent PEG-LDR9-αIgM”) or unpotentiated (“Neu5Gc Monovalent PEG-LDR6-αIgM” and “Neu5Gc Bivalent PEG-LDR6-αIgM”) for CD22 binding. As in FIG. 17 (Example 10) for the mouse B cell system, conjugates bearing Siglec Ligands that are unpotentiated for CD22 binding are fully active for BCR activation, while as before, potentiated binder conjugates show strong BCR suppression.

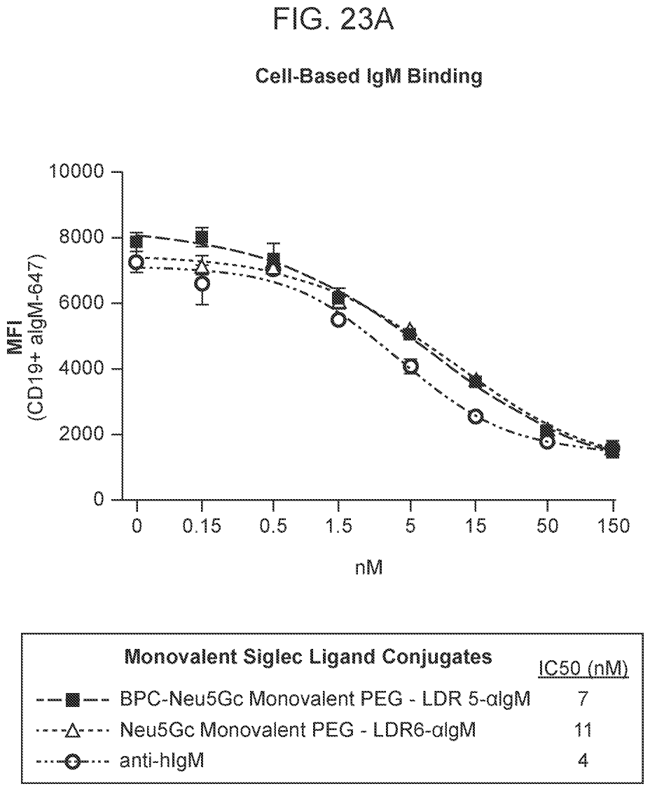

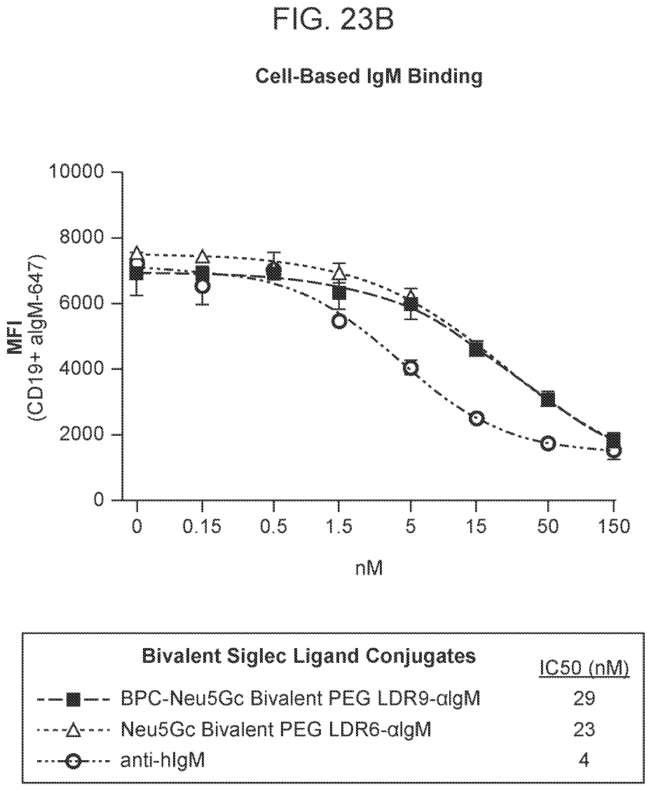

A BCR competition assay was used as in the above examples to control for perturbations in conjugate binding to the B cell receptor (FIG. 23). The test articles are identical to those used in the B cell activation experiment in FIG. 22. Potentiated and unpotentiated conjugates show equivalent binding IC50s to parental anti-IgM antibody, thus demonstrating a lack of perturbation in BCR binding for Siglec Ligand conjugates.

### Example 14: In Vivo Suppression of Anti-Drug Antibody in Mice Treated with Siglec Ligand-Adalimumab Conjugates

### Purpose

The purpose of this experiment was to test for suppression of immunogenicity in mice dosed with adalimumab-Siglec-2 Ligand conjugates. Parental adalimumab hIgG1 is highly immunogenic in mice, with a strong immunoglobulin response after a single 4 mg/kg dose. This and subsequent examples set out to corroborate the in vitro B cell suppressive effects shown in Examples 8 to 13 with in vivo assessment of effects on immunogenicity for Siglec Ligand conjugates with different proteins.

### Materials and Methods

Adalimumab hIgG1 and Adalimumab-Siglec Ligand conjugates were prepared as described in Example 7.

To evaluate the production of antibodies specific to adalimumab and/or adalimumab-Siglec Ligand conjugates, C57BL/6 mice were immunized through intravenous injection with adalimumab or Siglec Ligand-conjugated adalimumab. On study day −1, animals were randomized into treatment groups based on body weight. On study day 0, animals were bled for baseline serum and then injected IV with adalimumab or the adalimumab-Siglec Ligand conjugates. The individual antigens were prepared by making a 0.8 mg/ml antigen solution in sterile buffered saline. Animals were then injected with 0.1 ml (^{˜}4 ml/kg) of the 0.8 mg/ml antigen via the tail vein. The total dose based on a 20 g mouse would be 4 mg/kg. Animals were bled via the retro-orbital sinus weekly throughout the study under inhaled isoflurane anesthesia. On study day, 28 animals were anesthetized with inhaled isoflurane anesthesia and then bled via cardiac puncture and then sacrificed by cervical dislocation. Whole blood was collected into Microvette EDTA capillary collection tubes (Sarstedt Inc) and then further processed following the manufacturer's instructions for serum collection. Samples were stored at −80 C until analysis was performed.

ADA assays were performed on 96-well assay plates (Nunc Plates, Black 96-Well Immuno Plates, Thermo Scientific, 437111) coated with antigen, as follows. A mixture of adalimumab and adalimumab conjugates was coated at 5 μg/ml of each, with 100 μL/well. All coated antigens were diluted in PBS pH 7.2 and incubated overnight at 4° C. The following day, plate coating solution was removed, and plates were blocked with 200 μL/well of 3% BSA, 20 μM EDTA, 0.1% Tween-20 in PBS for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature, then washed with PBS buffer with 0.05% Tween-20. After washing, 100 μL of 1:2500 diluted Donkey Anti-Mouse IgG(H+L)-HRP (SouthernBiotech, 6411-05) was added and incubated for 1 hour at room temperature. After washing the assay plates, 100 μL of QuantaBlu Substrate Solution (Thermo Scientific, 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. Titers were determined by the dilution of serum that gives a 2× OD above background.

### Results and Conclusions

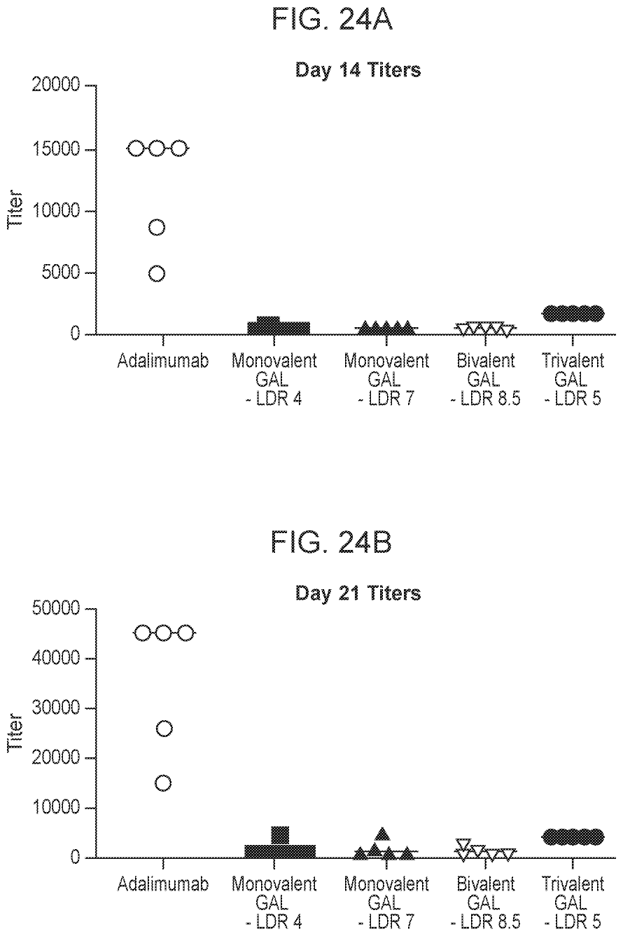

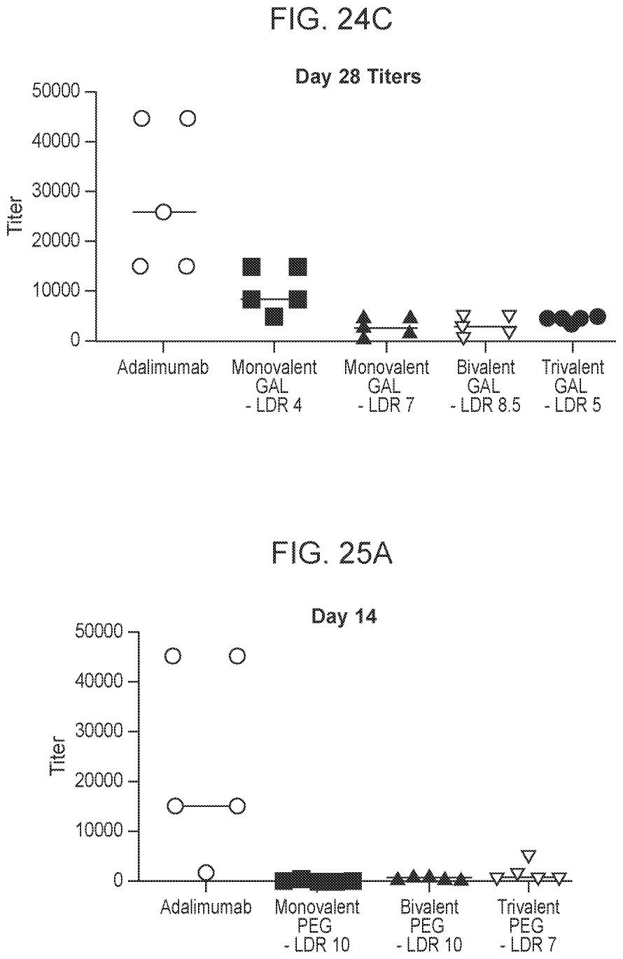

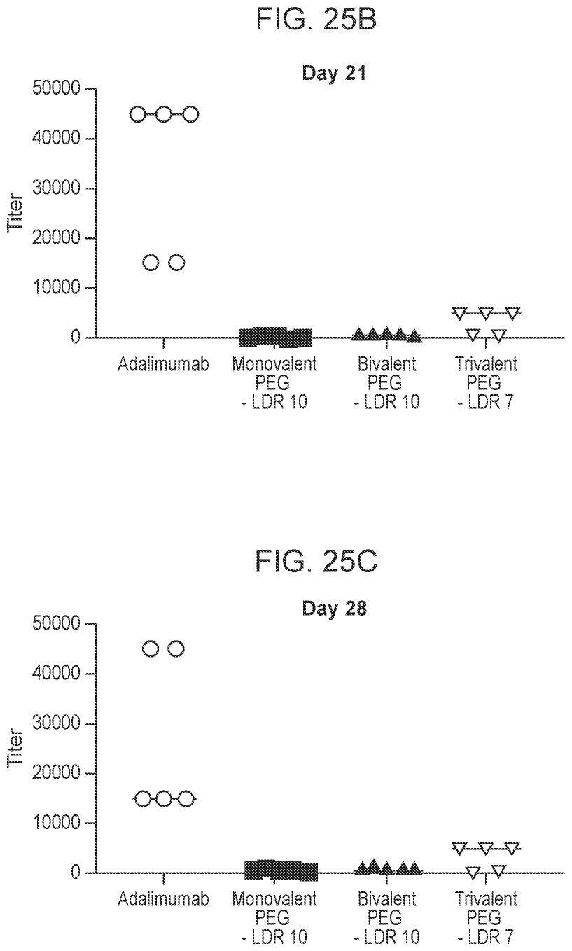

FIG. 24 and FIG. 25 depict evaluations of anti-drug antibody responses in mice for adalimumab and Siglec Ligand-adalimumab conjugates. Conjugates bear BPC-Neu5Gc-based Siglec Ligand-Linker structures that are either monovalent, bivalent, or trivalent for Siglec Ligands, with either galactose- (FIG. 24) or PEG-containing (FIG. 25) linker structures. Mice received a single 4 mg/kg i.v. dose of adalimumab or adalimumab-Siglec Ligand conjugate. Serum IgG levels against adalimumab/adalimumab conjugates were measured at days 14, 21, and 28. For both GAL- and PEG-linker containing conjugates, where parental adalimumab hIgG1 shows high mouse IgG serum titers at days 14, 21, and 28, Siglec Ligand conjugates are all strongly suppressed for anti-drug titer, with some modest differences between monovalent, bivalent, and trivalent conjugates for the degree of suppression. In contract with the previously described in vitro pan-BCR activation assays, where trivalent conjugates were markedly stronger in BCR suppression, in vivo immunogenicity results show monovalent, bivalent, and trivalent conjugates to all be potent in suppression of anti-drug titer.

These results for suppression of immunogenicity in mice correlate with the in vitro B cell activation results in Examples 8 to 13; decoration of an immunogenic antibody with potentiated Siglec Ligands is sufficient to strongly suppress immunogenicity.

### Example 15: Pharmacokinetic Analysis of Adalimumab and Siglec Ligand-Conjugated Adalimumab in C57BL/6 Mice

### Purpose

The purpose of this experiment was to determine if there are perturbations in pharmacokinetics of Siglec Ligand-conjugate adalimumab preparations relative to parental adalimumab IgG.

### Materials and Methods

Pharmacokinetic monitoring was carried out in C57BL/6 mice following a single intravenous administration of adalimumab or the individual SigL-conjugated adalimumab antibodies. On study day −1, the mice were randomized by body weight into treatment groups. On study day 0, the animals were administered the test articles IV via the tail vein with 0.1 mL of a 0.8 mg/mL antigen solution in sterile buffered saline for a total dose of approximately 4 mg/kg. Animals were bled via the retro-orbital sinus weekly throughout the study under inhaled isoflurane anesthesia at 0.3-, 1-, 7-, 5-, 7- and 10-day post test-article administration. Whole blood was collected into Microvette K2EDTA capillary collection tube (Sarstedt Inc) and then further processed following manufactures instructions to collect plasma. Levels of adalimumab and SigL-conjugated adalimumab in plasma samples were measured using an AlphaLISA human IgG assay (Perkin Elmer) following manufacturer's protocols.

Using an orthogonal analytical method, PK analysis of Adalimumab in mouse serum was performed on a Biacore 8K+ at 25° C. Briefly, anti-human Fc Antibody from the Human Antibody Capture Kit from Cytiva (Cat #: 29234600) was amine coupled on flow cell 2 across all 8 channels for a final response of about 7000 RU. A calibration series for each test article was established by spiking stock concentrations into a 1:300 dilution of naïve mouse serum in running buffer (HBS-EP+) and serially diluting 1:1 from 1000 μg/mL down to 0.976 μg/mL. Test serum samples were diluted 1:300 in running buffer as well. Serum and calibration samples were injected across both surfaces for 400 seconds at 30 μL/min and allowed to dissociate for 120 seconds. Regeneration of the surfaces was achieved with 2 injections of 3 M MgCl₂ for 15 seconds at 30 μL/min. A report point was chosen 5 seconds after injection of the sample for analysis. Using this report point, a calibration curve was constructed in GraphPad Prism (Version 9.0.0) using a Sigmoidal, 4PL least squares fit, and test serum sample concentrations were interpolated from this curve.

### Results and Conclusions

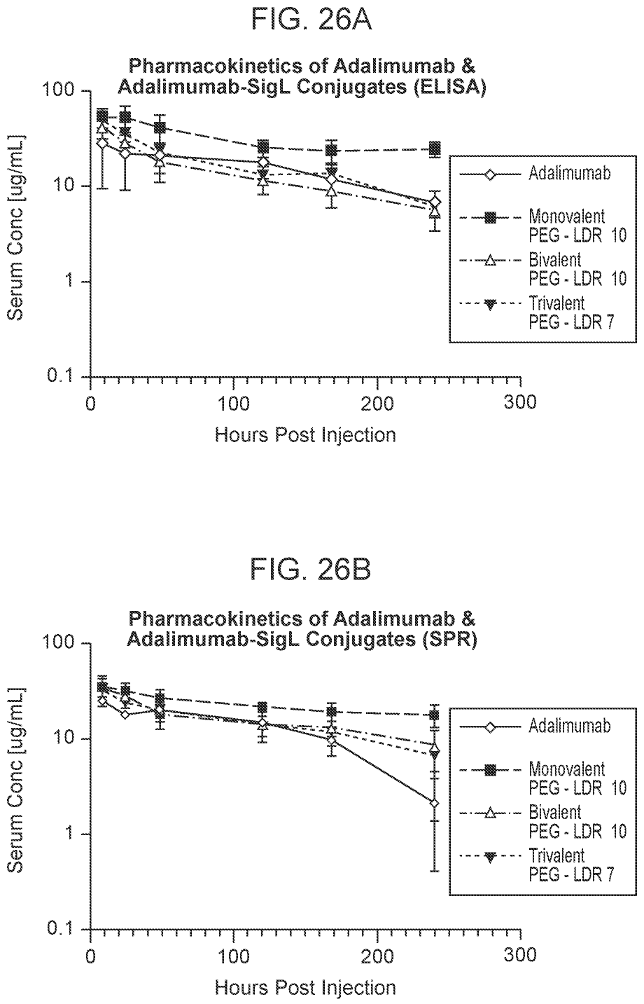

FIG. 26 depicts analysis of serum pharmacokinetics for adalimumab hIgG and Siglec Ligand-adalimumab hIgG1 conjugates in mice. Conjugates bear BPC-Neu5Gc-based Siglec Ligand-Linker structures that are either monovalent, bivalent, or trivalent for Siglec Ligands, with a PEG-containing linker structure. Test articles are identical to those used in Example 13 (FIG. 25). After intravenous dosing, serum samples were analyzed for test article concentrations by anti-human IgG Fc ELISA (FIG. 26A) and SPR (surface plasmon resonance, FIG. 26B).

Adalimumab IgG and adalimumab conjugates show equivalent serum PK profiles as measured by the two independent assay systems (ELISA and SPR). It should be noted that PK study was run at the same dose (4 mg/kg) as the immunogenicity study in Example 14. These results therefore rule out a lack of drug exposure as the basis for suppressed immunogenicity in Example 14, and also derisks Siglec Ligand conjugates for perturbations in serum PK.

### Example 16: Binding Analysis of Adalimumab and Adalimumab Conjugates to TNF (SPR)

### Purpose

The purpose of this experiment was to evaluate adalimumab Siglec Ligand conjugates for perturbations in functional activity, as measured through affinity analysis of TNFα binding.

### Materials and Methods

Binding experiments of Adalimumab Conjugates were performed on a Biacore 8K+ using a Cytiva Protein A Chip Series S (Cat #29127555). Briefly, conjugates were diluted in running buffer (HBS-EP+) and scouted for a concentration yielding about 30 RU of response after 20 seconds of injection at 30 μL/min on the active surface (flow cell 2). Human TNFα was serially diluted in running buffer 1:1 from 100 nM to 0.1953 nM and then injected across both the reference (flow cell 1) and active surface (flow cell 2) for 120 seconds at 30 μL/min and allowed to dissociate for 600 s. Regeneration of the surfaces was achieved with a 30 second pulse of 10 mM Glycine, pH 1.5 at 30 μL/min. Sensorgrams were fitted to a 1:1 binding model in Biacore Insight Evaluation Software Version 3.0.11.15423.

### Results

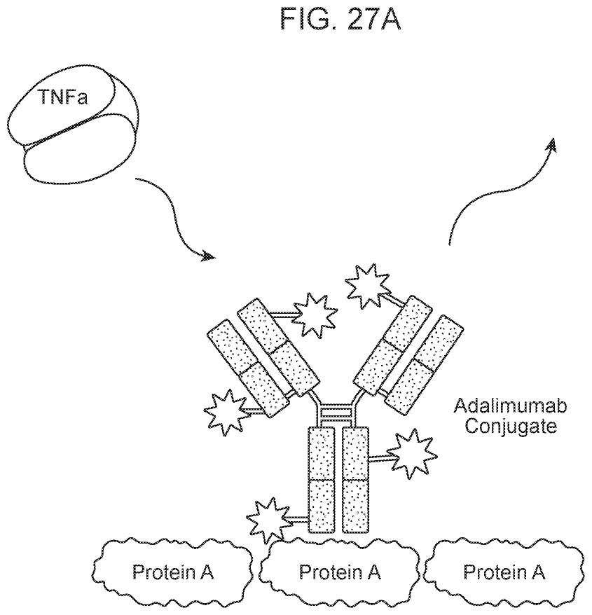

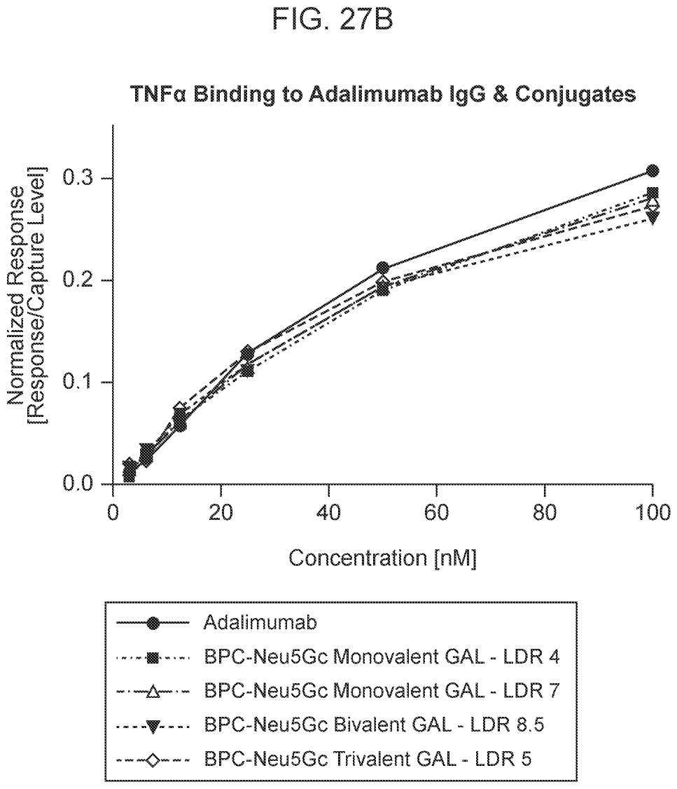

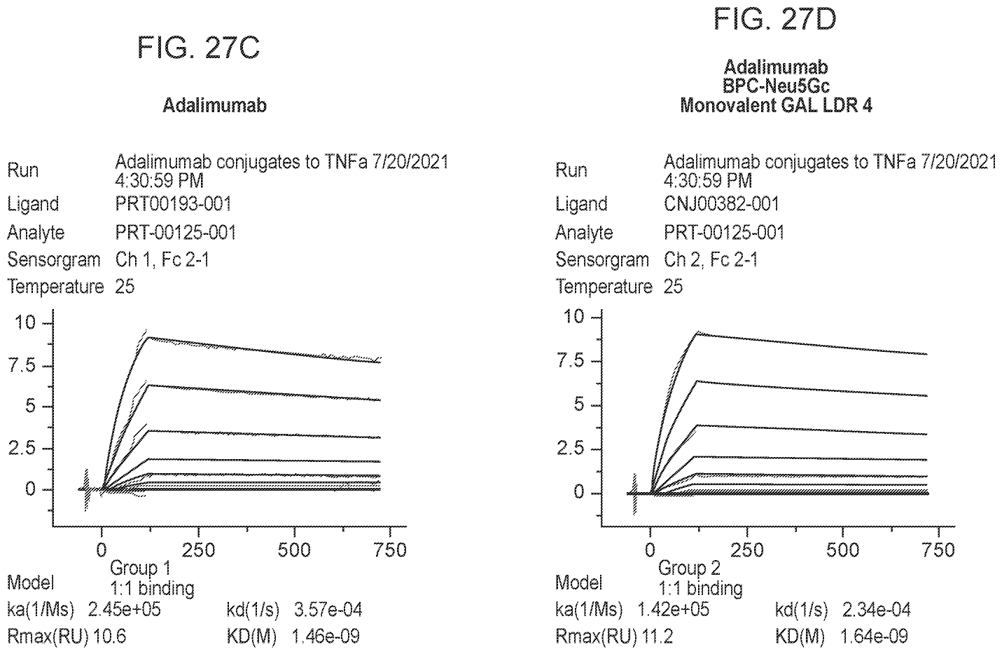

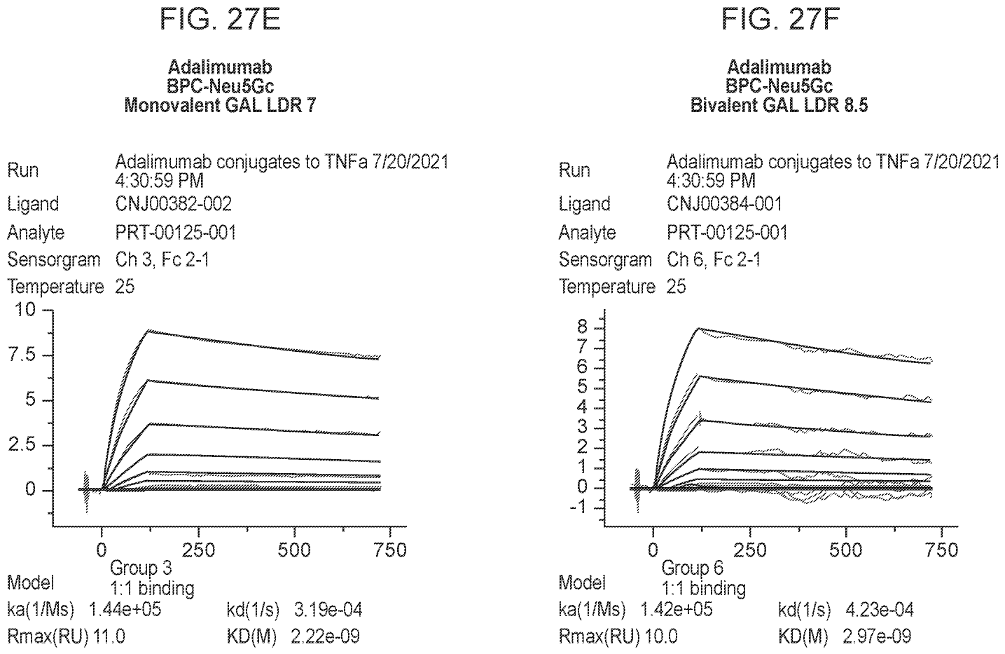

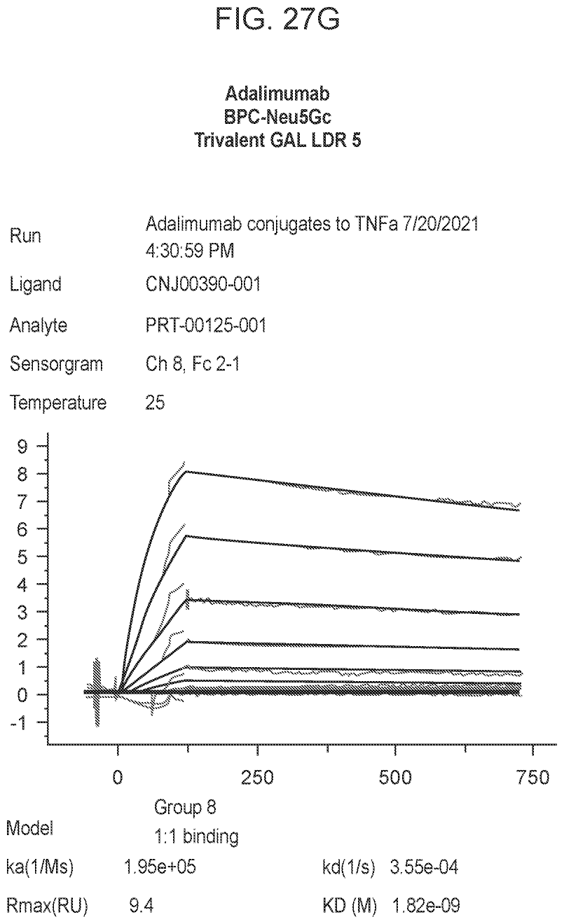

FIG. 27 depicts in vitro surface plasmon resonance (SPR) analysis of TNFα binding activity for adalimumab hIgG and Siglec Ligand-adalimumab hIgG1 conjugates. FIG. 27A is a schematic for the SPR assay protocol, with TNFα analyte binding to Protein A chip-immobilized adalimumab or adalimumab-Siglec Ligand conjugate. TNFα concentration was varied to evaluate concentration dependence, binding kinetics, and affinity. FIG. 27B shows the concentration dependencies of binding at the end of the sensorgram association phase (RUₘₐₓ). FIG. 27C-G are individual sensorgrams for Adalimumab (FIG. 27C), Adalimumab BPC-Neu5Gc Monovalent GAL LDR 4 (FIG. 27D), Adalimumab BPC-Neu5Gc Monovalent GAL LDR 7 (FIG. 27E), Adalimumab BPC-Neu5Gc Bivalent GAL LDR 8.5 (FIG. 27F), and Adalimumab BPC-Neu5Gc Trivalent GAL LDR 5 (FIG. 27G). Fit kinetic and affinity parameters are summarized in TABLE 3.

TABLE 3
TNFα - Adalimumab/Adalimumab-Siglec Ligand Conjugate SPR Binding Assays

| Test Article | ka (1/Ms) | kd (1/s) | KD (M) | Rmax (RU) |
|---|---|---|---|---|
| Adalimumab | 2.45E+05 | 3.57E−04 | 1.46E−09 | 10.6 |
| Adalimumab BPC-Neu5Gc Monovalent GAL - LDR 4 | 1.42E+05 | 2.34E−04 | 1.64E−09 | 11.2 |
| Adalimumab BPC-Neu5Gc Monovalent GAL - LDR 7 | 1.44E+05 | 3.19E−04 | 2.22E−09 | 11 |
| Adalimumab BPC-Neu5Gc Bivalent GAL - LDR 8.5 | 1.42E+05 | 4.23E−04 | 2.97E−09 | 10 |
| Adalimumab BPC-Neu5Gc Trivalent GAL - LDR 5 | 1.95E+05 | 3.55E−04 | 1.82E−09 | 9.4 |

### Conclusions

Adalimumab and Siglec Ligand-Adalimumab conjugates all bind TNFα with similar apparent affinities and percent activities. Adalimumab-Siglec Ligand conjugates are therefore unperturbed in their TNFα binding properties. Importantly, these results show that functional activity in biotherapeutic can be maintained while ablating immunogenicity through Siglec Ligand conjugation.

### Example 17: In Vivo Suppression of Anti-Drug Antibody in Mice Treated with Siglec Ligand-Adalimumab Conjugates Requires CD22/Siglec-2 Ligands

### Purpose

The purpose of this experiment was to test for the importance of Siglec-2 binding activity in adalimumab conjugates for the suppressed immunogenicity seen in mice dosed with adalimumab-Siglec-2 Ligand conjugates. This study uses the same asialo, PEG-based linker structures from Example 10 (FIG. 17 and FIG. 18) that do not bind Siglec-2 for negative control adalimumab-linker conjugates.

### Materials and Methods

Adalimumab hIgG1 and Adalimumab-Siglec Ligand conjugates were prepared as described in Example 7.

To evaluate the production of antibodies specific to adalimumab and/or adalimumab-Siglec Ligand conjugates, C57BL/6 mice were immunized through intravenous injection with adalimumab or Siglec Ligand-conjugated adalimumab. On study day −1, animals were randomized into treatment groups based on body weight. On study day 0, animals were bled for baseline serum and then injected IV with adalimumab or the adalimumab-Siglec Ligand conjugates. The individual antigens were prepared by making a 0.8 mg/ml antigen solution in sterile buffered saline. Animals were then injected with 0.1 ml (^{˜}4 ml/kg) of the 0.8 mg/ml antigen via the tail vein. The total dose based on a 20 g mouse would be 4 mg/kg. Animals were bled via the retro-orbital sinus weekly throughout the study under inhaled isoflurane anesthesia. On study day, 28 animals were anesthetized with inhaled isoflurane anesthesia and then bled via cardiac puncture and then sacrificed by cervical dislocation. Whole blood was collected into Microvette EDTA capillary collection tubes (Sarstedt Inc) and then further processed following the manufacturer's instructions for serum collection. Samples were stored at −80 C until analysis was performed.

ADA assays were performed on 96-well assay plates (Nunc Plates, Black 96-Well Immuno Plates, Thermo Scientific, 437111) coated with antigen, as follows. A mixture of adalimumab and adalimumab conjugates was coated at 5 μg/ml of each, with 100 μL/well. All coated antigens were diluted in PBS pH 7.2 and incubated overnight at 4° C. The following day, plate coating solution was removed, and plates were blocked with 200 μL/well of 3% BSA, 20 μM EDTA, 0.1% Tween-20 in PBS for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature, then washed with PBS buffer with 0.05% Tween-20. After washing, 100 μL of 1:2500 diluted Donkey Anti-Mouse IgG(H+L)-HRP (SouthernBiotech, 6411-05) was added and incubated for 1 hour at room temperature. After washing the assay plates, 100 μL of QuantaBlu Substrate Solution (Thermo Scientific, 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. Titers were determined by the dilution of serum that gives a 2× OD above background.

### Results and Conclusions

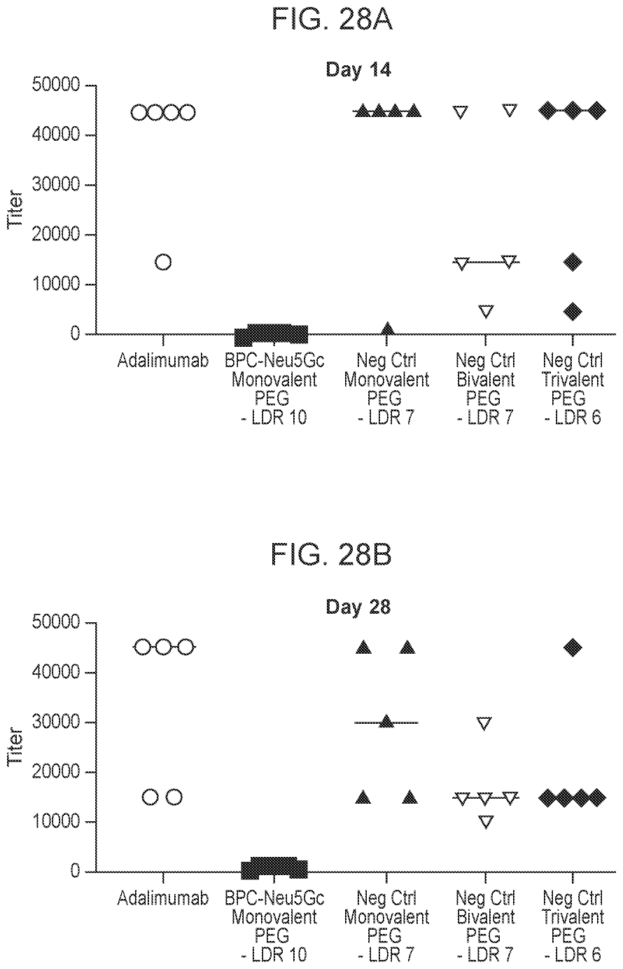

FIG. 28 depicts evaluation of anti-drug antibody responses in mice for 1) adalimumab, 2) a potentiated Siglec Ligand-adalimumab conjugate (“BPC-Neu5Gc Monovalent PEG LDR 10”), and 3) negative control, non-Siglec-2-binding linker conjugates (“Neg Ctrl Monovalent PEG LDR 7”, “Neg Ctrl Bivalent PEG LDR 7”, and “Neg Ctrl Trivalent PEG LDR 6”). Mice received a single 4 mg/kg i.v. dose of adalimumab, adalimumab-Siglec Ligand conjugate, or adalimumab-negative control linker conjugate. Serum IgG levels against adalimumab/adalimumab conjugates were measured at days 14 and 28. FIG. 28A and FIG. 28B show individual animal serum IgG titer values (n=5 mice) at days 14 and 28, respectively. While the potentiated Siglec Ligand-adalimumab conjugate shows complete suppression of immunogenicity at days 14 and 28, negative control adalimumab conjugates are completely unperturbed in immunogenicity. The data are consistent with the in vitro mouse B cell activation assay observations in Example 10 (FIG. 17 and FIG. 18), where asialo conjugates are inactive for suppression of BCR activation. Importantly, these results show that the suppressed immunogenicity of adalimumab with the platform Siglec Ligand conjugate technology depends critically on the Siglec Ligand-binding chemical matter.

### Example 18: In Vivo Suppression of Anti-Drug Antibody in Mice Treated with Siglec Ligand-Adalimumab Conjugates Requires Potentiated Binding of CD22/Siglec-2 Ligands

### Purpose

Where Example 10 (FIG. 16) showed the importance of potentiated (vs unpotentiated) Siglec-2 binding for the suppression of BCR activation, the purpose of this experiment was to test the importance of potentiated Siglec-2 binding for suppression of adalimumab immunogenicity.

### Materials and Methods

Adalimumab hIgG1 and Adalimumab-Siglec Ligand conjugates were prepared as described in Example 7.

To evaluate the production of antibodies specific to adalimumab and/or adalimumab-Siglec Ligand conjugates, C57BL/6 mice were immunized through intravenous injection with adalimumab or Siglec Ligand-conjugated adalimumab. On study day −1, animals were randomized into treatment groups based on body weight. On study day 0, animals were bled for baseline serum and then injected IV with adalimumab or the adalimumab-Siglec Ligand conjugates. The individual antigens were prepared by making a 0.8 mg/ml antigen solution in sterile buffered saline. Animals were then injected with 0.1 ml (^{˜}4 ml/kg) of the 0.8 mg/ml antigen via the tail vein. The total dose based on a 20 g mouse would be 4 mg/kg. Animals were bled via the retro-orbital sinus weekly throughout the study under inhaled isoflurane anesthesia. On study day, 28 animals were anesthetized with inhaled isoflurane anesthesia and then bled via cardiac puncture and then sacrificed by cervical dislocation. Whole blood was collected into Microvette EDTA capillary collection tubes (Sarstedt Inc) and then further processed following the manufacturer's instructions for serum collection. Samples were stored at −80 C until analysis was performed.

ADA assays were performed on 96-well assay plates (Nunc Plates, Black 96-Well Immuno Plates, Thermo Scientific, 437111) coated with antigen, as follows. A mixture of adalimumab and adalimumab conjugates was coated at 5 μg/ml of each, with 100 μL/well. All coated antigens were diluted in PBS pH 7.2 and incubated overnight at 4° C. The following day, plate coating solution was removed, and plates were blocked with 200 μL/well of 3% BSA, 20 μM EDTA, 0.1% Tween-20 in PBS for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature, then washed with PBS buffer with 0.05% Tween-20. After washing, 100 μL of 1:2500 diluted Donkey Anti-Mouse IgG(H+L)-HRP (SouthernBiotech, 6411-05) was added and incubated for 1 hour at room temperature. After washing the assay plates, 100 μL of QuantaBlu Substrate Solution (Thermo Scientific, 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. Titers were determined by the dilution of serum that gives a 2× OD above background.

### Results and Conclusions

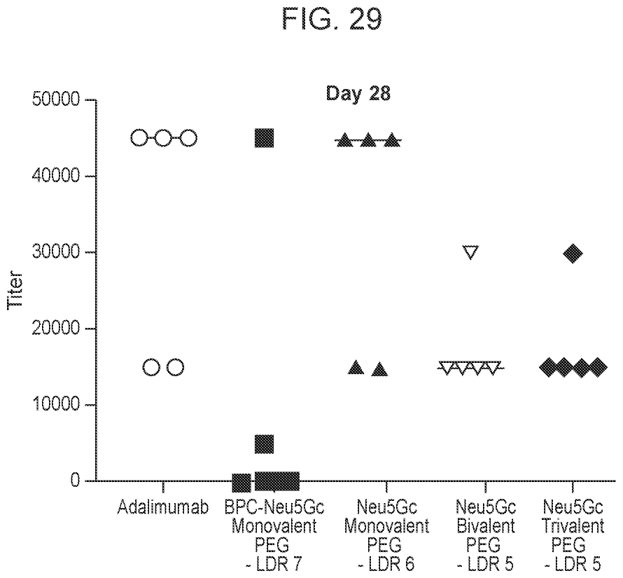

FIG. 29 depicts evaluation of anti-drug antibody responses in mice for 1) adalimumab, 2) a potentiated Siglec Ligand-adalimumab conjugate (“BPC-Neu5Gc Monovalent PEG LDR 7”), and 3) Non-potentiated Siglec Ligand-adalimumab conjugates (“Neu5Gc Monovalent PEG LDR 6”, “Neu5Gc Bivalent PEG LDR 5”, and “Neu5Gc Trivalent PEG LDR 5”). Mice received a single 4 mg/kg i.v. dose of adalimumab IgG or conjugate. Serum IgG levels against adalimumab/adalimumab conjugates were measured at day 28. Individual animal serum IgG titer values (n=5 mice) are shown for day 28.

In this experiment, the potentiated Siglec Ligand conjugate was strongly suppressed for immunogenicity, while the three different (monovalent, bivalent, and trivalent) conjugates bearing unpotentiated, Neu5Gc linker structures were highly immunogenic. These data are consistent with the in vitro B cell assay result in Example 10 (FIG. 17 and FIG. 18), where potentiated Siglec-2 binding was critical for suppression of BCR activation. Potentiation is thus critical not only for suppression of BCR activation in a pan-B cell activation assay, but also for the in vivo suppression of immunogenicity to adalimumab-Siglec Ligand conjugates. From this result, it is expected that hypersialylated forms of adalimumab (bearing native, unpotentiated Neu5Gc or Neu5Ac structures) will be unperturbed for immunogenicity in vivo.

### Example 19: In Vivo Suppression of Anti-Drug Antibody in Mice Treated with Siglec Ligand-Adalimumab Conjugates is Specific for Siglec Ligand-Conjugates and not a Co-Administered Non-Conjugate Immunogen

### Purpose

The purpose of this experiment was to test for effects of adalimumab-Siglec Ligand conjugate administration on mouse humoral responses to a separate, standard immunogen, Hen Egg White Lysozyme. Where other methods to decrease immunogenicity of an administered drug could conceivably lead to a general suppression of immunogenicity responses, the mechanism of action of Siglec Ligand-conjugates is expected to suppress immunogenicity only for the conjugate and not other co-dosed immunogens.

### Materials and Methods

Adalimumab hIgG1 and Adalimumab-Siglec Ligand conjugates were prepared as described in Example 7.

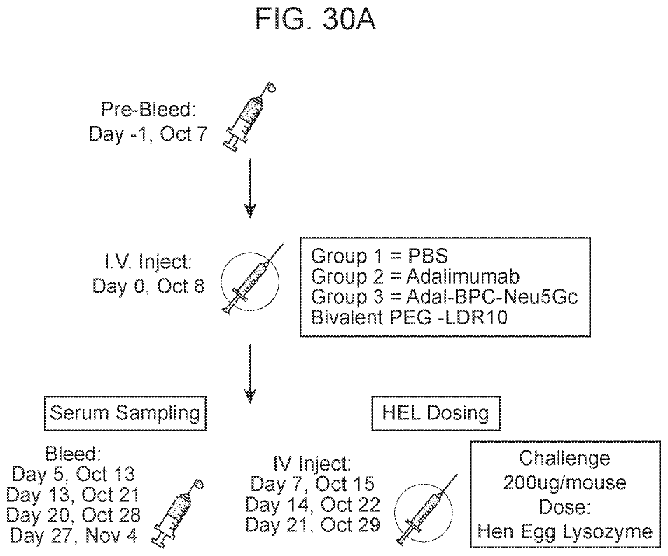

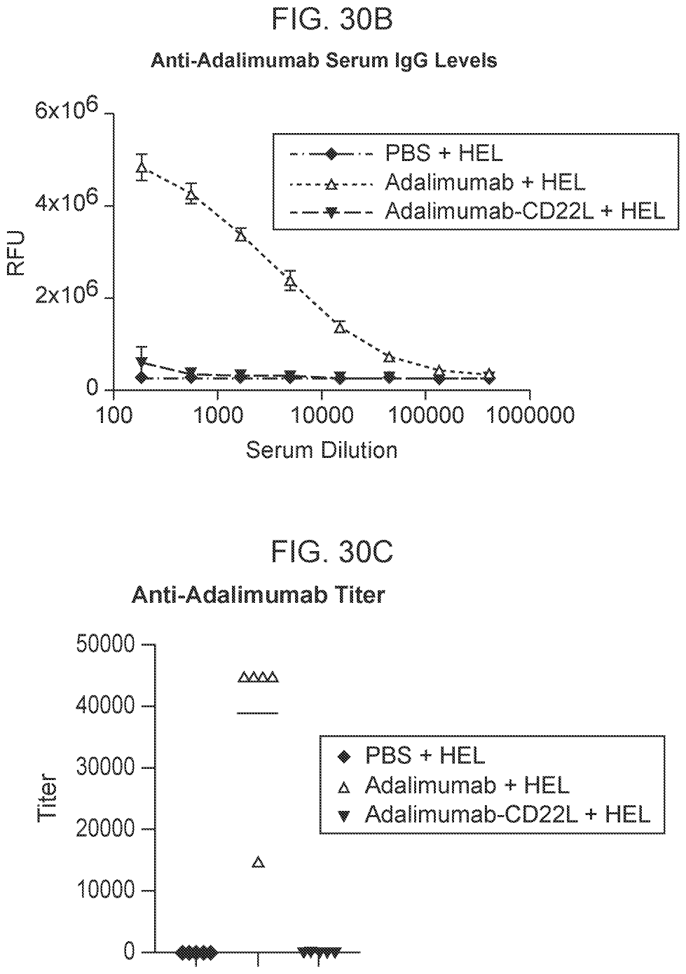

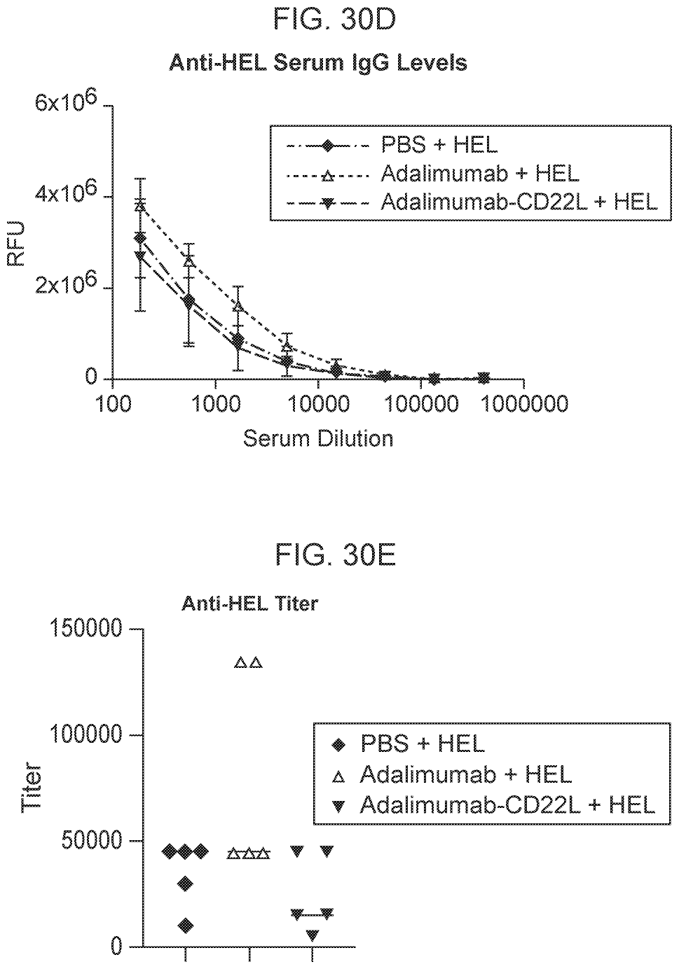

FIG. 30A shows the study scheme for administration of adalimumab, adalimumab-Siglec Ligand conjugate, or PBS vehicle control, followed by administration of Hen Egg White Lysozyme (HEL), to C57BL/6 mice. On study day −1, animals were randomized into treatment groups based on body weight. On study day 0, animals were bled for baseline serum and then injected IV with adalimumab, adalimumab-Siglec Ligand conjugate, or PBS vehicle. The individual adalimumab-based antigens were prepared by making a 0.8 mg/ml antigen solution in sterile buffered saline. Animals were then injected with 0.1 ml (^{˜}4 ml/kg) of the 0.8 mg/ml antigen via the tail vein. The total dose based on a 20 g mouse would be 4 mg/kg. Mice were subsequently administered 200 μg HEL on days 7, 14, and 21. Whole blood was collected into Microvette EDTA capillary collection tubes (Sarstedt Inc) and then further processed following the manufacturer's instructions for serum collection. Samples were stored at −80 C until analysis was performed.

ADA assays were performed on 96-well assay plates (Nunc Plates, Black 96-Well Immuno Plates, Thermo Scientific, 437111) coated with antigen as follows. For evaluation of adalimumab serum titers, a first set of plates was coated with a mixture of adalimumab and conjugates (100 μL/well, 5 μg/mL each). For evaluation of HEL serum titers, a second set of plates was coated with 100 μL/well of 5 μg/mL purified Lysozyme (HEL, Sigma, L4919-1G). The following day, plate coating solution was removed, and plates were blocked with 200 μL/well of 3% BSA, 20 μM EDTA, 0.1% Tween-20 in PBS for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature. Plates were washed with PBS buffer with 0.05% Tween-20. After washing, 100 μL of 1:2500 diluted Donkey Anti-Mouse IgG(H+L)-HRP (SouthernBiotech, 6411-05) was added and incubated for 1 hour at room temperature. After washing the assay plates, 100 μL of QuantaBlu Substrate Solution (Thermo Scientific, 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. Titers were determined by the dilution of serum that gives a 2× OD above background.

### Results and Conclusions

FIG. 30 depicts evaluation of IgG immune responses in mice dosed with different combinations of 4 mg/kg adalimumab, adalimumab-Siglec Ligand conjugate (Adalimumab-BPC-Neu5Gc Bivalent PEG LDR 10), or PBS vehicle (day 0), and subsequent weekly dosing with 200 μg hen egg white lysozyme (HEL) (days 7, 14, 21, and 28). Mice serum IgG titers were measured separately against adalimumab/adalimumab-Siglec Ligand conjugate and HEL. Day 28 serum IgG levels are shown for anti-adalimumab/adalimumab conjugate (FIG. 30B) and HEL (FIG. 30D). Titers are shown for Anti-adalimumab/adalimumab conjugates (FIG. 30C) and HEL (FIG. 30E).

As in previous experiments (Examples 14, 17 and 18), where adalimumab immunization induced a strong IgG response, adalimumab-Siglec Ligand conjugate was very strongly suppressed for an antibody response. In contrast, the same animals, when dosed 1, 2, and 3 weeks later with HEL (and where previous PK analysis shows Adalimumab conjugate would still be present in dosed animals) showed completely unperturbed IgG responses to HEL. Therefore, the Siglec Ligand-conjugate technology platform described here affects only directly-conjugated molecules and does not affect B cell antibody responses to unrelated antigens, i.e., Siglec Ligand-conjugates are unique as specific, not general, immunosuppressants.

### Example 20: In Vivo Suppression of Specific Antibody Response in Mice Treated with Siglec Ligand-Hen Egg White Lysozyme Conjugates

### Purpose

The purpose of this experiment was to test for suppression of immunogenicity in mice dosed with a second immunogen, HEL. As shown in Example 19, HEL is highly immunogenic in mice. This example thus sets out to expand the demonstration of in vivo immunogenicity suppression beyond adalimumab hIgG1 to other immunogens.

### Materials and Methods

HEL (Sigma, L4919-1G) and HEL-Siglec Ligand conjugates (prepared from Sigma, L4919-1G) were prepped using the methods described in Example 7. HEL-BPC-Neu5Gc was prepared to high purity (cGE, anSEC, LC/MS) with an LDR of 1.6.

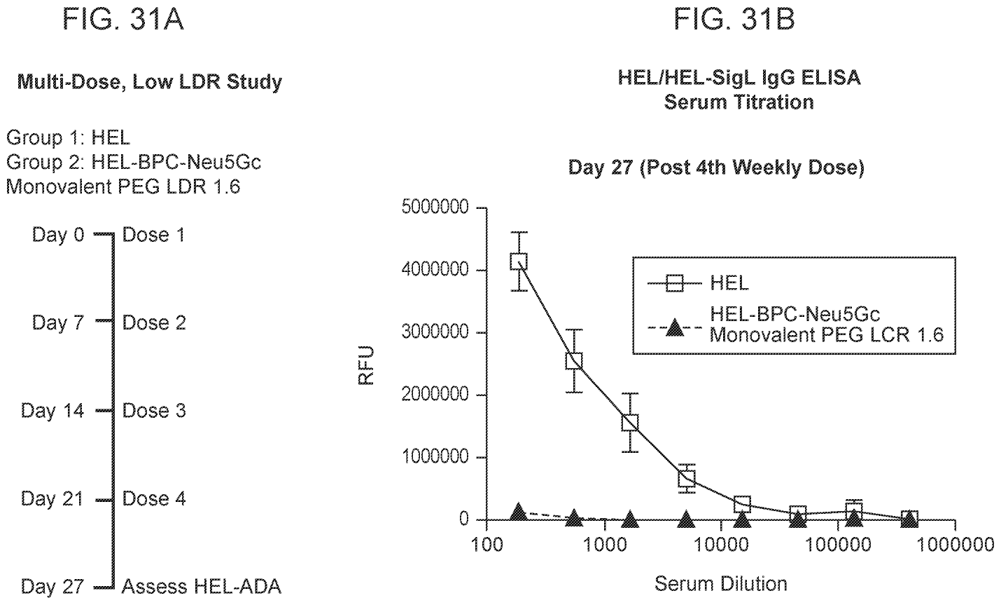

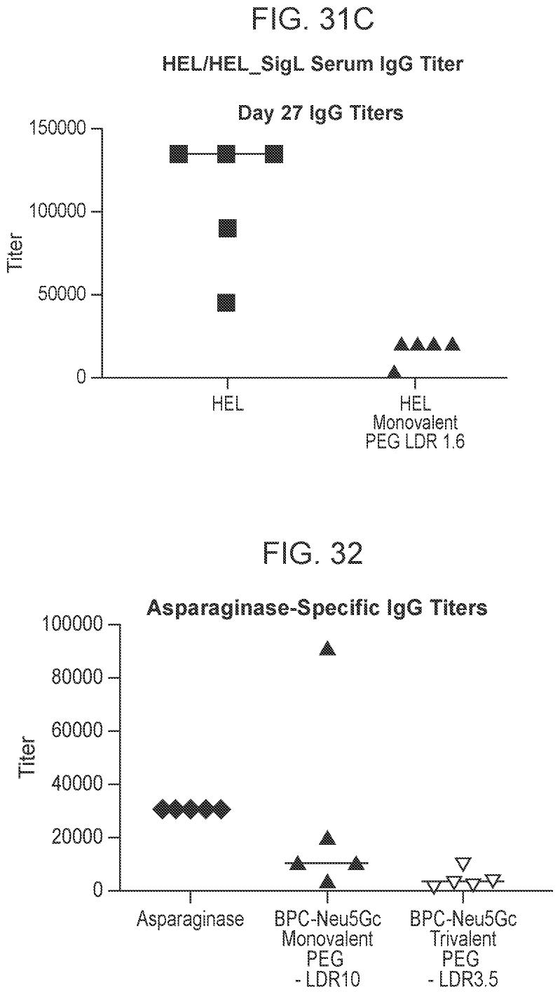

FIG. 31A shows the study scheme for administration of Hen Egg White Lysozyme (HEL) and HEL-Siglec Ligand conjugates to C57BL/6 mice. On study day −1, animals were randomized into treatment groups based on body weight. On study day 0, animals were bled for baseline serum and then injected IV with 200 μg/mouse HEL or HEL-Siglec Ligand conjugate (HEL-BPC-Neu5Gc Monovalent PEG-LDR 1.6). Mice were subsequently administered 200 μg/mouse HEL on days 7, 14, and 21. Serum IgG levels against HEL/HEL conjugate were measured at day 27. Whole blood was collected into Microvette EDTA capillary collection tubes (Sarstedt Inc) and then further processed following the manufacturer's instructions for serum collection. Samples were stored at −80 C until analysis was performed.

ADA assays were performed on 96-well assay plates (Nunc Plates, Black 96-Well Immuno Plates, Thermo Scientific, 437111) coated with antigen as follows. Plates were coated with 100 μL/well of a mixture of 5 μg/mL of purified Lysozyme (HEL, Sigma, L4919-1G) with 5 μg/ml HEL-Siglec Ligand conjugate. Coated antigens were diluted in PBS pH 7.2 and incubated overnight at 4° C. The following day, the coating solution was removed, and plates were blocked with 200 μL/well of 3% BSA, 20 μM EDTA, 0.1% Tween-20 in PBS for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature, then washed with PBS buffer+0.05% Tween-20. After washing, 100 μL of 1:2500 diluted Donkey Anti-Mouse IgG(H+L)-HRP (SouthernBiotech, 6411-05) was added and incubated for 1 hour at room temperature. After washing the assay plates, 100 μL of QuantaBlu Substrate Solution (Thermo Scientific, 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. Titers were determined by the dilution of serum that gives a 2× OD above background.

### Results and Conclusions

FIG. 31 depicts the evaluation of anti-drug antibody responses in mice for HEL and a monovalent Siglec Ligand-HEL conjugate (“HEL-BPC-Neu5Gc Monovalent PEG LDR 1.6). Mice had received 4 weekly 200 μg i.v. doses of HEL or HEL conjugate (days 0, 7, 14, and 21). FIG. 31B shows the IgG level dilution series from serum samples. FIG. 31C shows the individual animal day 27 serum IgG titer values (n=5 mice).

While mice administered repeat (4) doses of HEL showed vigorous IgG responses to HEL, those administered Siglec Ligand-HEL conjugates were completely suppressed for immunogenicity. Therefore, the suppression of immunogenicity seen with Siglec Ligand conjugate technology, seen previously with adalimumab, is seen also with an unrelated antigen, HEL. Suppression of immunogenicity with the described CD22-coengagin technology therefore applies to other, non-antibody immunogens, and could apply to many unrelated drug modalities.

### Example 21: In Vivo Anti-Asparaginase Response is Suppressed for Asparaginase-Siglec Ligand Conjugates

### Purpose

The purpose of this experiment was to test for suppression of immunogenicity in mice dosed with a third immunogen, the E. coli-derived enzyme, Asparaginase. This example thus sets out to expand the demonstration of in vivo immunogenicity suppression beyond adalimumab hIgG1 and HEL to other immunogens. As in previous examples, potentiated Siglec Ligand conjugates were produced and purified, this time using purified Asparaginase enzyme.

### Materials and Methods

To evaluate the production of antibodies specific to asparaginase, BALB/c mice were immunized with recombinant asparaginase or the individual Siglec Ligand-asparaginase conjugate preparations. On study day −1, animals were randomized into treatment groups based on body weight. On study day 0, animals were bled for baseline serum. Mice were injected i.v. with 15 μg recombinant asparaginase or Siglec Ligand-conjugated asparaginase preparation per mouse on days 0, 7, 14, and 21. Animals were bled via the retro-orbital sinus weekly throughout the study under inhaled isoflurane anesthesia. Whole blood was collected into Microvette EDTA capillary collection tube (Sarstedt Inc) and then further processed following manufactures instructions to collect serum. Samples where then stored at −80 C until analysis was performed.

ADA assays were performed on 96-well assay plates (Nunc Plates, Black 96-Well Immuno Plates, Thermo Scientific, 437111) coated with 100 μL/well of 5 μg/mL asparaginase in PBS. Plates were incubated overnight at 4° C. The following day, coating solution was removed and plates were blocked with 200 μL/well of 3% BSA, 20 μM EDTA, 0.1% Tween-20 in PBS for 1 hour at room temperature. The serum samples were diluted 1:185 in 3% BSA, 20 μM EDTA, 0.1% Tween-20 in PBS and added in three-fold serial dilutions. Plates were incubated 1 hour at room temperature. Plates were washed with PBS buffer with 0.05% Tween-20. After washing 100 μL of 1:2500 diluted Donkey Anti-Mouse IgG(H+L)-HRP (SouthernBiotech, 6411-05) was added and incubated for 1 hour at room temperature. After washing the assay plates, 100 μL of QuantaBlu Substrate Solution (Thermo Scientific, 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. Titers were determined by the dilution of serum that gives a 2× OD above background.

### Results

FIG. 32 depicts the evaluation of anti-drug antibody responses in mice for recombinant asparaginase enzyme and asparaginase-Siglec Ligand conjugates (“Asn'ase BPC-Neu5Gc Monovalent PEG-LDR 10” and “Asn'ase BPC-Neu5Gc Trivalent PEG-LDR 3.5”). BALB/c (n=5 per group) mice were dosed weekly (4 times) with 15 μg Asparaginase or Asparaginase conjugate. Anti-Asparaginase IgG titers were measured at day 28 by ELISA assay. Titers are shown for each test article. Mice treated with Siglec Ligand-Asparaginase, especially those treated with trivalent Siglec Ligand-asparaginase conjugate, showed suppressed anti-asparaginase responses.

### EMBODIMENTS OF THE INVENTION

Embodiments of the present invention include, but are not limited to the following clauses.

1. An engineered hypoimmunogenic biotherapeutic, comprising a biotherapeutic which has been engineered to comprise a modified Sialic acid-binding immunoglobulin-type lectin (Siglec) ligand profile relative to a corresponding unengineered biotherapeutic while retaining therapeutic activity.

2. The engineered hypoimmunogenic biotherapeutic according to clause 1, wherein the Siglec ligand profile comprises an elevated amount of one or more Siglec ligands covalently bound to the engineered hypoimmunogenic biotherapeutic relative to the corresponding unengineered biotherapeutic.

3. The engineered hypoimmunogenic biotherapeutic according to clause 1 or 2, wherein one or more of the Siglec ligands is a ligand for a B cell-associated Siglec.

4. The engineered hypoimmunogenic biotherapeutic according to clause 3, wherein the B-cell associated Siglec is selected from the group consisting of Siglec-2 (CD22), Siglec-5 (CD170), Siglec-6, Siglec-9 (CD329) and Siglec-10 (Siglec G).

5. The engineered hypoimmunogenic biotherapeutic according to any one of clauses 1-4, wherein the Siglec ligand comprises a sialic acid.

6. The engineered hypoimmunogenic biotherapeutic according to clause 5, wherein the sialic acid is a naturally occurring sialic acid.

7. The engineered hypoimmunogenic biotherapeutic according to clause 6, wherein the Siglec ligand is a naturally occurring Siglec ligand.

8. The engineered hypoimmunogenic biotherapeutic according to clause 6, wherein the Siglec ligand is a non-naturally occurring Siglec ligand.

9. The engineered hypoimmunogenic biotherapeutic according to clause 8, wherein the naturally occurring sialic acid is covalently bound to the biotherapeutic.

10. The engineered hypoimmunogenic biotherapeutic according to clause 8, wherein the non-naturally occurring Siglec ligand further comprises a non-naturally occurring linker.

11. The engineered hypoimmunogenic biotherapeutic according to clause 10, wherein the non-naturally occurring Siglec ligand consists essentially of the naturally occurring sialic acid bound to the non-naturally occurring linker.

12. The engineered hypoimmunogenic biotherapeutic according to clause 10 or 11, wherein the linker does not comprise a saccharide.

13. The engineered hypoimmunogenic biotherapeutic according to clause 5, wherein the Siglec ligand is a non-naturally occurring Siglec ligand that comprises a non-naturally occurring sialic acid.

14. The engineered hypoimmunogenic biotherapeutic according to clause 13, wherein the non-naturally occurring sialic acid is covalently bound to the biotherapeutic.

15. The engineered hypoimmunogenic biotherapeutic according to clause 13, wherein the non-naturally occurring Siglec ligand further comprises a non-naturally occurring linker.

16. The engineered hypoimmunogenic biotherapeutic according to clause 15, wherein the non-naturally occurring Siglec ligand consists essentially of the non-naturally occurring sialic acid covalently bound to the non-naturally occurring linker.

17. The engineered hypoimmunogenic biotherapeutic according to clause 15 or 16, wherein the linker does not comprise a saccharide.

18. The engineered hypoimmunogenic biotherapeutic according to clause 5, wherein the Siglec ligand comprises two sialic acids and a linker, wherein the linker is a branched linker and the two sialic acids are attached to the linker.

19. The engineered hypoimmunogenic biotherapeutic according to clause 5, wherein the Siglec ligand comprises three sialic acids and a linker, wherein the linker is a branched linker and the three sialic acids are attached to the linker.

20. The engineered hypoimmunogenic biotherapeutic according to clause 18 or 19, wherein the linker does not comprise a natural saccharide.

21. The engineered hypoimmunogenic biotherapeutic according to any one of clauses 1-4, wherein the Siglec ligand comprises a Siglec binding peptide.

22. The engineered hypoimmunogenic biotherapeutic according to clause 21, wherein the Siglec binding peptide comprises RNDYTE.

23. The engineered hypoimmunogenic biotherapeutic according to any one of clauses 1-22, wherein the hypoimmunogenic biotherapeutic comprises Siglec ligands for both Siglec-2 and Siglec-10.

24. The engineered hypoimmunogenic biotherapeutic according to any one of clauses 1-23, wherein the engineered hypoimmunogenic biotherapeutic comprises one or more Siglec ligands and the corresponding unengineered immunogenic biotherapeutic comprises less Siglec ligands than the engineered hypoimmunogenic biotherapeutic.

25. The engineered hypoimmunogenic biotherapeutic according to clause 24, wherein the engineered hypoimmunogenic biotherapeutic comprises one or more Siglec ligands and the corresponding unengineered immunogenic biotherapeutic comprises no Siglec ligands.

26. The engineered hypoimmunogenic biotherapeutic according to any one of clauses 1-23, wherein the hypoimmunogenic biotherapeutic comprises 2-fold more Siglec ligand than a corresponding unengineered immunogenic biotherapeutic that induces an antibody response in an individual administered the biotherapeutic.

27. The engineered hypoimmunogenic biotherapeutic according to any one of clauses 1-23, wherein the hypoimmunogenic biotherapeutic comprises 3-fold more Siglec ligand than a corresponding unengineered biotherapeutic that induces an antibody response in an individual administered the biotherapeutic.

28. The engineered hypoimmunogenic biotherapeutic according to any one of clauses 1-23, wherein the hypoimmunogenic biotherapeutic comprises 5-fold more Siglec ligand than a corresponding unengineered biotherapeutic that induces an antibody response in an individual administered the biotherapeutic.

29. The engineered hypoimmunogenic biotherapeutic according to any one of clauses 1-23, wherein the hypoimmunogenic biotherapeutic comprises 10-fold more Siglec ligand than a corresponding unengineered biotherapeutic that induces an antibody response in an individual administered the biotherapeutic.

30. The engineered hypoimmunogenic biotherapeutic according to any one of clauses 1-23, wherein the engineered hypoimmunogenic biotherapeutic further comprises an elevated amount of an ASGPR ligand covalently bound to the engineered hypoimmunogenic biotherapeutic relative to the corresponding unengineered biotherapeutic.

31. The engineered hypoimmunogenic biotherapeutic according to clause 30, wherein the ASGPR ligand is a naturally occurring GalNAc.

32. The engineered hypoimmunogenic biotherapeutic according to clause 30, wherein the ASGPR ligand is a GalNAc glycomimetic.

33. The engineered hypoimmunogenic biotherapeutic according to any one of clauses 1-32, wherein the hypoimmunogenic biotherapeutic elicits a biotherapeutic-specific antibody titer that is 50% of the biotherapeutic-specific antibody titer that would be elicited by a corresponding unengineered biotherapeutic or less in an individual administered the biotherapeutic.

34. The engineered hypoimmunogenic biotherapeutic according to clause 33, wherein the hypoimmunogenic biotherapeutic is administered to an individual for 1 month or more.

35. The engineered hypoimmunogenic biotherapeutic according to clause 33, wherein the hypoimmunogenic biotherapeutic is administered to an individual for 3 months or more.

36. The engineered hypoimmunogenic biotherapeutic according to clause 33, wherein the hypoimmunogenic biotherapeutic is administered to an individual for 6 months or more.

37. The engineered hypoimmunogenic biotherapeutic according to clause 33, wherein the hypoimmunogenic biotherapeutic is administered to an individual for 1 year or more.

38. The engineered hypoimmunogenic biotherapeutic according to any one of clauses 33-37, wherein the administration to the individual is weekly.

39. The engineered hypoimmunogenic biotherapeutic according to any one of clauses 33-37, wherein the administration to the individual is biweekly.

40. The engineered hypoimmunogenic biotherapeutic according to any one of clauses 33-37, wherein the administration to the individual is monthly.

41. The engineered hypoimmunogenic biotherapeutic according to any one of clauses 33-37, wherein the administration to the individual is quarterly or semi-annually.

42. The engineered hypoimmunogenic biotherapeutic according to any one of clauses 33-37, wherein the administration to the individual is annually or bi-annually.

43. The engineered hypoimmunogenic biotherapeutic according to any one of clauses 33-37, wherein the ADA titer is measured 8 weeks after the last administration of the biotherapeutic.

44. The engineered hypoimmunogenic biotherapeutic according to any one of clauses 1-43, wherein the biotherapeutic is a protein.

45. The engineered hypoimmunogenic biotherapeutic according to clause 44, wherein the protein is selected from the group consisting of an antibody, an enzyme, a chimeric protein, and a viral particle.

46. The engineered hypoimmunogenic biotherapeutic according to clause 45, wherein the antibody is selected from the group consisting of a monoclonal antibody, a bispecific antibody, an scFv, a Fab, a camelid, or a nanobody.

47. The engineered hypoimmunogenic biotherapeutic according to clause 46, wherein the antibody is selected from the group consisting of adalimumab, infliximab, cetuximab, natalizumab, moxetumomab pasudotox, atezolizumab, nivolumab, abciximab, Brentuximab, Certolizumab pegol, elotuzumab, benralizumab, vedolizumab, galcanezumab, rituximab, alemtuzumab, dupilumab, golimumab, obinutuzumab, tildrakizumab, erenumab, mepolizumab, tamucirumab, ranibizumab, ustekinumab, reslizumab, ipilimumab, alirocumab, belimumab, panitumumab, avelumab, necitumumab, mogamulizumab, olaratumab, brodalumab, eculizumab, pertuzumab, pembrolizumab, and tocilizumab.

48. The engineered hypoimmunogenic biotherapeutic according to clause 44, wherein the protein is selected from the group consisting of erythropoietin, thrombopoietin, human growth hormone, tissue factor, IFNβ-1b, IFNβ-1a, IL-2 or the IL-2 mimetic aldesleukin, exenatide, albiglutide, alefacept, palifermin, and belatacept.

49. The engineered hypoimmunogenic biotherapeutic according to clause 45, wherein the enzyme is selected from the group consisting of asparaginase Erwinia chrysanthemi, phenylalanine ammonia-lyase, alpha-galactosidase A, acid α-glucosidase (GAA), glucocerebrosidase (GCase), aspartylglucosaminidase (AGA), alpha-L-iduronidase, iduronate sulfatase, sulfaminase, α-N-acetylglucosaminidase (NAGLU), heparin acetyle CoA: α-glucosaminide N-acetyltransferase (HGSNAT), N-acetylglucosamine 6-sulfatase (GNS), N-glucosamine 3-O-sulfatase (arylsulfatase G or ARSG), N-acetylgalactosamine 6-sulfatase, beta-galactosidase, N-acetylgalactosamine 4-sulfatase, beta-glucuronidase, Factor VIII, Factor IX, palmitoyl protein thioesterase (PPT1), Tripeptidyl peptidase (TPP1), Pseudomonas elastase (PaE), Pseudomonas alkaline protease (PaAP), and Streptococcal pyrogenic exotoxin B (SpeB).

50. The engineered hypoimmunogenic biotherapeutic according to clause 45, wherein the viral particle is selected from a recombinant adeno-associated virus (rAAV) particle, a recombinant human adenovirus (rHAdV) particle, a recombinant Herpes Simplex Virus (rHSV) particle, a recombinant papillomavirus (PV) particle, a recombinant polyomavirus particle, a recombinant vaccinia virus particle, a recombinant cytomegalovirus (CMV) particle, a recombinant baculovirus particle, a recombinant human papillomavirus (HPV) particle, and a recombinant retrovirus particle.

51. The engineered hypoimmunogenic biotherapeutic according to clause 50, wherein the rAAV particle comprises a capsid VP1 protein selected from the group consisting of an AAV1, AAV2, AAV3b, AAV4, AAV5, AAV6, AAV7, AAV8, AAV9, AAVrh10, AAV11, AAV12, and AAV13 VP1 protein, or a variant thereof.

52. The engineered hypoimmunogenic biotherapeutic according to clause 50, wherein the recombinant comprises a capsid protein from a human adenovirus particle selected from the group consisting of a recombinant HAdV-A, HAdV-B, HAdV-C, HAdV-D, HAdV-E, HAdV-F, and HAdV-G or a variant thereof.

53. The engineered hypoimmunogenic biotherapeutic according to clause 50, wherein the recombinant HSV particle is selected from a recombinant HSV1 or HSV2 particle or a variant thereof.

54. The engineered hypoimmunogenic biotherapeutic according to clause 50, wherein the recombinant retrovirus particle is selected from the group consisting of a lentivirus particle, human immunodeficiency virus (HIV) particle, Simian immunodeficiency virus (SIV) particle, Feline immunodeficiency virus (FIV) particle, Puma lentivirus (PLV) particle, Equine infectious anemia virus (EIAV) particle, Bovine immunodeficiency virus (BIV) particle, Caprine arthritis encephalitis virus particle, gammaretrovirus particle, and murine leukemia virus (MLV) particle, or variant or pseudotyped virus thereof.

55. A method of making a hypoimmunogenic biotherapeutic, the method comprising covalently attaching a sialic acid to a biotherapeutic to create an engineered hypoimmunogenic biotherapeutic.

56. The method according to clause 55, wherein the covalently attaching comprises sialylation by engineered biosynthesis.

57. The method according to clause 55, wherein the covalently attaching comprises sialylation by chemical conjugation.

58. The method according to any one of clauses 55-57, wherein the chemical conjugation of the sialic acid is to a glycan of the biotherapeutic.

59. The method according to clause 58, wherein the chemical conjugation of the sialic acid to the glycan of the biotherapeutic results in a covalent bond between the sialic acid and the glycan.

60. The method according to clause 58, wherein the chemical conjugation of the sialic acid to the glycan of the biotherapeutic incorporates a linker between the sialic acid and the glycan.

61. The method according to any one of clauses 55-57, wherein the chemical conjugation of the sialic acid is to an amino acid of the biotherapeutic.

62. The method according to clause 61, wherein the chemical conjugation of the sialic acid to the amino acid of the biotherapeutic results in a covalent bond between the sialic acid and the amino acid.

63. The method according to clause 61, wherein the chemical conjugation of the sialic acid to the amino acid of the biotherapeutic incorporates a linker between the sialic acid and the amino acid.

64. The method according to any one of clauses 55-63, wherein the sialic acid is a naturally occurring sialic acid.

65. The method according to any one of clauses 55-63, wherein the sialic acid is a non-naturally occurring sialic acid.

66. The method according to clause 55, wherein the covalently attaching comprises the insertion of a Siglec binding peptide or polypeptide into the amino acid sequence of the biotherapeutic by genetic engineering.

67. The method according to clause 66, wherein the Siglec binding peptide is RNDYTE.

68. The method according to any one of clauses 55-67, wherein the covalently attaching results in the generation of a Siglec ligand.

69. The method according to clause 68, wherein the Siglec ligands is a ligand for a B cell-associated Siglec.

70. The method according to clause 69, wherein the B-cell associated Siglec is selected from the group consisting of Siglec-2 (CD22), Siglec-5 (CD170), Siglec-6, Siglec-9 (CD329) and Siglec-10 (Siglec G).

71. The method according to any one of clauses 55-70, wherein the amount of sialic acid associated with the biotherapeutic is increased 2-fold or more following the covalent attaching.

72. The method according to any one of clauses 55-70, wherein the amount of Siglec ligand associated with the biotherapeutic is increased 5-fold or more following the covalent attaching.

73. The method according to any one of clauses 55-70, wherein the amount of Siglec ligand associated with the biotherapeutic is increased 10-fold or more following the covalent attaching.

74. The method according to any one of clauses 55-73, wherein the engineered hypoimmunogenic biotherapeutic further comprises an elevated amount of an ASGPR ligand covalently bound to the engineered hypoimmunogenic biotherapeutic relative to the corresponding unengineered biotherapeutic.

75. The method according to clause 74, wherein the ASGPR ligand is a naturally occurring GalNAc.

76. The method according to clause 74, wherein the ASGPR ligand is a GalNAc glycomimetic.

77. The method according to any one of clauses 55-76, wherein the biotherapeutic is a protein.

78. The method according to clause 77, wherein the protein is selected from the group consisting of an antibody, an enzyme, a chimeric protein, and a viral particle.

79. The method according to clause 78, wherein the antibody is selected from the group consisting of a monoclonal antibody, a bispecific antibody, an scFv, a Fab, a camelid, or a nanobody.

80. The method according to clause 78 or 79, wherein the antibody is selected from the group consisting of adalimumab, infliximab, cetuximab, natalizumab, moxetumomab pasudotox, atezolizumab, nivolumab, abciximab, Brentuximab, Certolizumab pegol, elotuzumab, benralizumab, vedolizumab, galcanezumab, rituximab, alemtuzumab, dupilumab, golimumab, obinutuzumab, tildrakizumab, erenumab, mepolizumab, tamucirumab, ranibizumab, ustekinumab, reslizumab, ipilimumab, alirocumab, belimumab, panitumumab, avelumab, necitumumab, mogamulizumab, olaratumab, brodalumab, eculizumab, pertuzumab, pembrolizumab, and tocilizumab.

81. The method according to clause 77, wherein the protein is selected from the group consisting of erythropoietin, thrombopoietin, human growth hormone, tissue factor, IFNβ-1b, IFNβ-1a, IL-2 or the IL-2 mimetic aldesleukin, exenatide, albiglutide, alefacept, palifermin, and belatacept.

82. The method according to clause 78, wherein the enzyme is selected from the group consisting of asparaginase Erwinia chrysanthemi, phenylalanine ammonia-lyase, alpha-galactosidase A, acid α-glucosidase (GAA), glucocerebrosidase (GCase), aspartylglucosaminidase (AGA), alpha-L-iduronidase, iduronate sulfatase, sulfaminase, α-N-acetylglucosaminidase (NAGLU), heparin acetyle CoA: α-glucosaminide N-acetyltransferase (HGSNAT), N-acetylglucosamine 6-sulfatase (GNS), N-glucosamine 3-O-sulfatase (arylsulfatase G or ARSG), N-acetylgalactosamine 6-sulfatase, beta-galactosidase, N-acetylgalactosamine 4-sulfatase, beta-glucuronidase, Factor VIII, Factor IX, palmitoyl protein thioesterase (PPT1), and Tripeptidyl peptidase (TPP1).

83. The method according to clause 78, wherein the viral particle is selected from a recombinant adeno-associated virus (rAAV) particle, a recombinant human adenovirus (rHAdV) particle, a recombinant Herpes Simplex Virus (rHSV) particle, a recombinant papillomavirus (PV) particle, a recombinant polyomavirus particle, a recombinant vaccinia virus particle, a recombinant cytomegalovirus (CMV) particle, a recombinant baculovirus particle, a recombinant human papillomavirus (HPV) particle, and a recombinant retrovirus particle.

84. A pharmaceutical composition, comprising: an engineered hypoimmunogenic biotherapeutic according to any one of clauses 1-54 or a composition manufactured according to any one of clauses 55-83; and a pharmaceutical excipient.

85. A method of treating an individual suffering from a disorder or disease that could be treated by the administration of a biotherapeutic agent, the method comprising administering to the individual the pharmaceutical composition according to clause 84 in an amount effective to treat the disorder or disease, wherein the pharmaceutical composition elicits a reduced anti-drug antibody titer relative to the unengineered biotherapeutic.

86. The method according to clause 85, wherein the disease is a chronic immune disease selected from the group consisting of rheumatoid arthritis, psoriatic arthritis, ankylosing spondylitis, Crohn's disease, ulcerative colitis, psoriasis, hidradenitis suppurativa, uveitis, and juvenile idiopathic arthritis, wherein the administering comprises administering to the individual an engineered hypoimmunogenic TNFα-specific antibody selected from adalimumab and infliximab in an amount effective to treat the chronic immune disease.

87. The method according to clause 85, wherein the disease is a leukemia, wherein the administering comprises administering to the individual an engineered hypoimmunogenic asparaginase from Erwinia chrysanthemi in an amount effective to treat the cancer.

88. The method according to clause 85, wherein the disease is multiple sclerosis, wherein the administering comprises administering to the individual an engineered hypoimmunogenic natalizumab, an engineered hypoimmunogenic IFNβ-1b, or an engineered hypoimmunogenic IFNβ-1a in an amount effective to treat the multiple sclerosis.

89. The method according to clause 85, wherein the disorder is an antibody response to a transplanted tissue, wherein the administering comprises administering to the individual an engineered hypoimmunogenic IdeS in an amount effective to suppress the antibody response to the transplanted tissue.

90. The method according to clause 89, wherein the transplanted tissue is an allogeneic graft.

91. The method according to clause 89, wherein the transplanted tissue is a xenograft.

92. The method according to any one of clauses 86-91, wherein the tissue is selected from kidney, heart, lung, liver, pancreas, trachea, vascular tissue, skin, bone, cartilage, adrenal tissue, fetal thymus, and cornea.

93. The method according to clause 85, wherein the disorder is Type 2 Diabetes, wherein the administering comprises administering to the individual an engineered hypoimmunogenic exenatide or engineered hypoimmunogenic albiglutide in an amount effective to treat the disorder.

94. The method according to clause 85, wherein the disorder is an enzyme deficiency, wherein the administering comprises administering to the individual an engineered hypoimmunogenic enzyme in an amount effective to treat the deficiency.

95. The method according to clause 94, wherein the enzyme deficiency is a deficiency for an enzyme selected from the group consisting of phenylalanine ammonia-lyase (PKU), alpha-galactosidase A (for Fabry), acid α-glucosidase (GAA, for Pompe), glucocerebrosidase (GCase, for Gaucher), aspartylglucosaminidase (AGA, for Aspartylglucosaminuria), alpha-L-iduronidase (for MPS I), iduronate sulfatase (for MPS II), sulfaminase (MPS Ilila), α-N-acetylglucosaminidase (NAGLU, for MPS IIIB), heparin acetyle CoA: α-glucosaminide N-acetyltransferase (HGSNAT, for MPS IIIC), N-acetylglucosamine 6-sulfatase (GNS, for MPS IID), N-glucosamine 3-O-sulfatase (arylsulfatase G or ARSG, MPS IIIE), N-acetylgalactosamine 6-sulfatase (for MPS IVA), beta-galactosidase (for MPS IVB), N-acetylgalactosamine 4-sulfatase (for MPS VI), beta-glucuronidase (for MPS VI), Factor VIII (for hemophilia A), Factor IX (for hemophilia B), palmitoyl protein thioesterase (PPT1, for CLN1), Tripeptidyl peptidase (TPP1, for CLN2), and cystathionine beta synthase (CBS) deficiency.

96. The method according to clause 85, wherein the disorder is a monogenic disease, wherein the administering comprises administering to the individual an engineered hypoimmunogenic viral particle comprising a transgene encoding a therapeutic product in an amount effective to treat the disease.

97. The method according to any one of clauses 85-96, wherein the method further comprises:
drawing serum from the individual 8 weeks after administering the engineered hypoimmunogenic biotherapeutic and assessing the serum for biotherapeutic-specific antibodies, wherein the titer of biotherapeutic-specific antibodies is 50% of the titer that would be elicited by a corresponding unengineered biotherapeutic.

98. The method according to clause 97, wherein the titer of biotherapeutic-specific antibodies is 20% of the titer that would be elicited by a corresponding unengineered biotherapeutic.

99 The method according to clause 97, wherein the titer of biotherapeutic-specific antibodies is 5% of the titer that would be elicited by a corresponding unengineered biotherapeutic.

100. The method according to clause 97, wherein biotherapeutic-specific antibodies cannot be detected.

101. An engineered hypoimmunogenic biotherapeutic, comprising a biotherapeutic covalently bound to a nonnaturally occurring Siglec ligand, wherein the Siglec ligand comprises
a non-naturally occurring sialic acid selected from the group consisting of

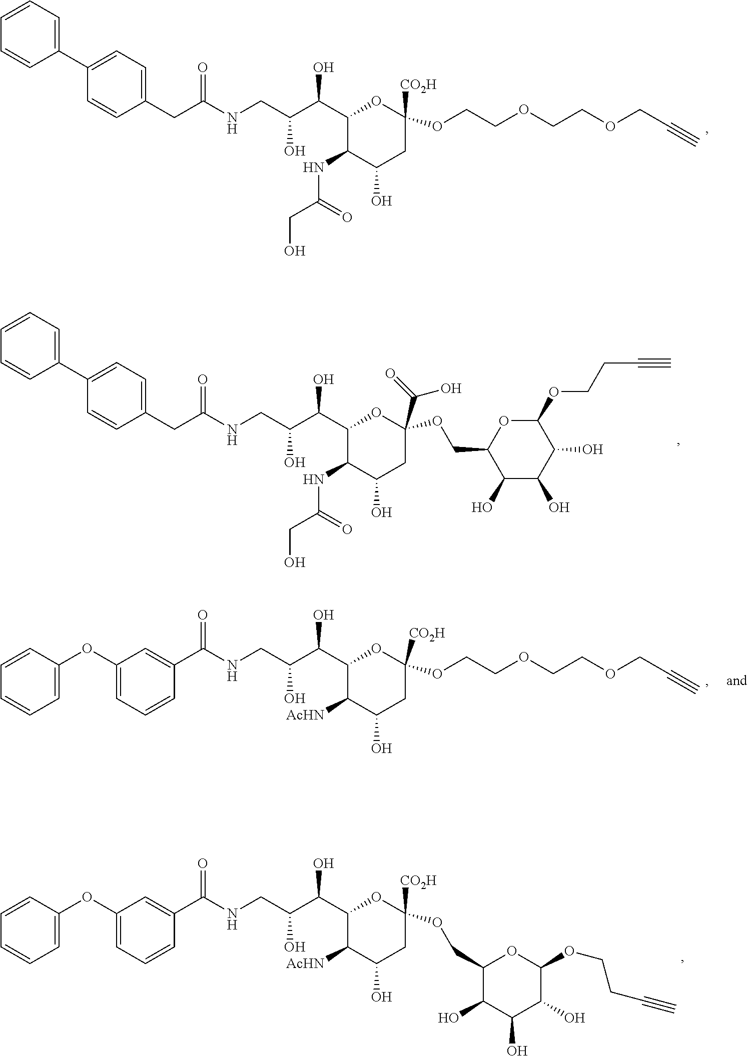 [SMILES: C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1 ; C#CCCO[C@@H]1O[C@H](CO[C@]2(C(=O)O)C[C@H](O)[C@@H](NC(=O)CO)[C@H]([C@H](O)[C@H](O)CNC(=O)Cc3ccc(-c4ccccc4)cc3)O2)[C@H](O)[C@H](O)[C@H]1O ; C#CCOCCOCCO[C@]1(C(=O)O)C[C@H](O)[C@@H](NC(C)=O)[C@H]([C@H](O)[C@H](O)CNC(=O)c2cccc(Oc3ccccc3)c2)O1 ; C#CCCO[C@@H]1O[C@H](CO[C@]2(C(=O)O)C[C@H](O)[C@@H](NC(C)=O)[C@H]([C@H](O)[C@H](O)CNC(=O)c3cccc(Oc4ccccc4)c3)O2)[C@H](O)[C@H](O)[C@H]1O]

and a linker selected from the group consisting of

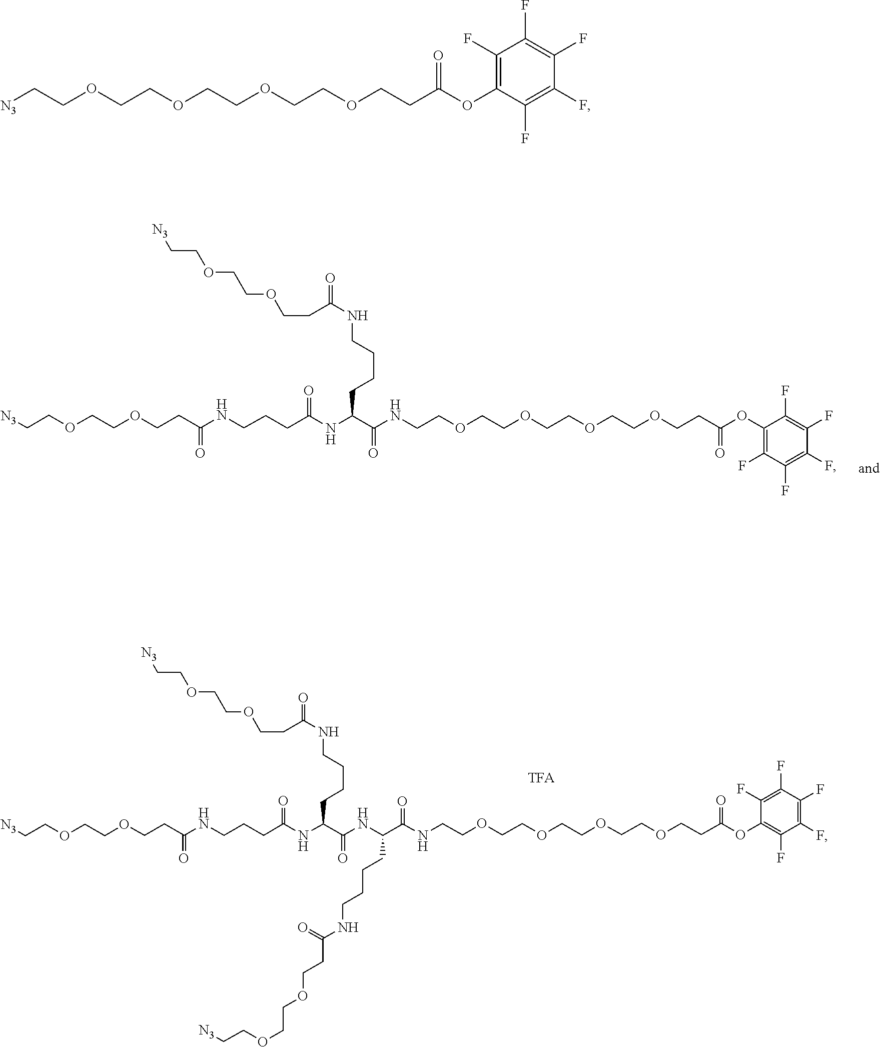 [SMILES: [N-]=[N+]=NCCOCCOCCOCCOCCC(=O)Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F]

wherein the linker attaches the sialic acid to the biotherapeutic.

102. The engineered hypoimmunogenic biotherapeutic according to clause 101, wherein the nonnaturally occurring Siglec ligand is selected from the group consisting of

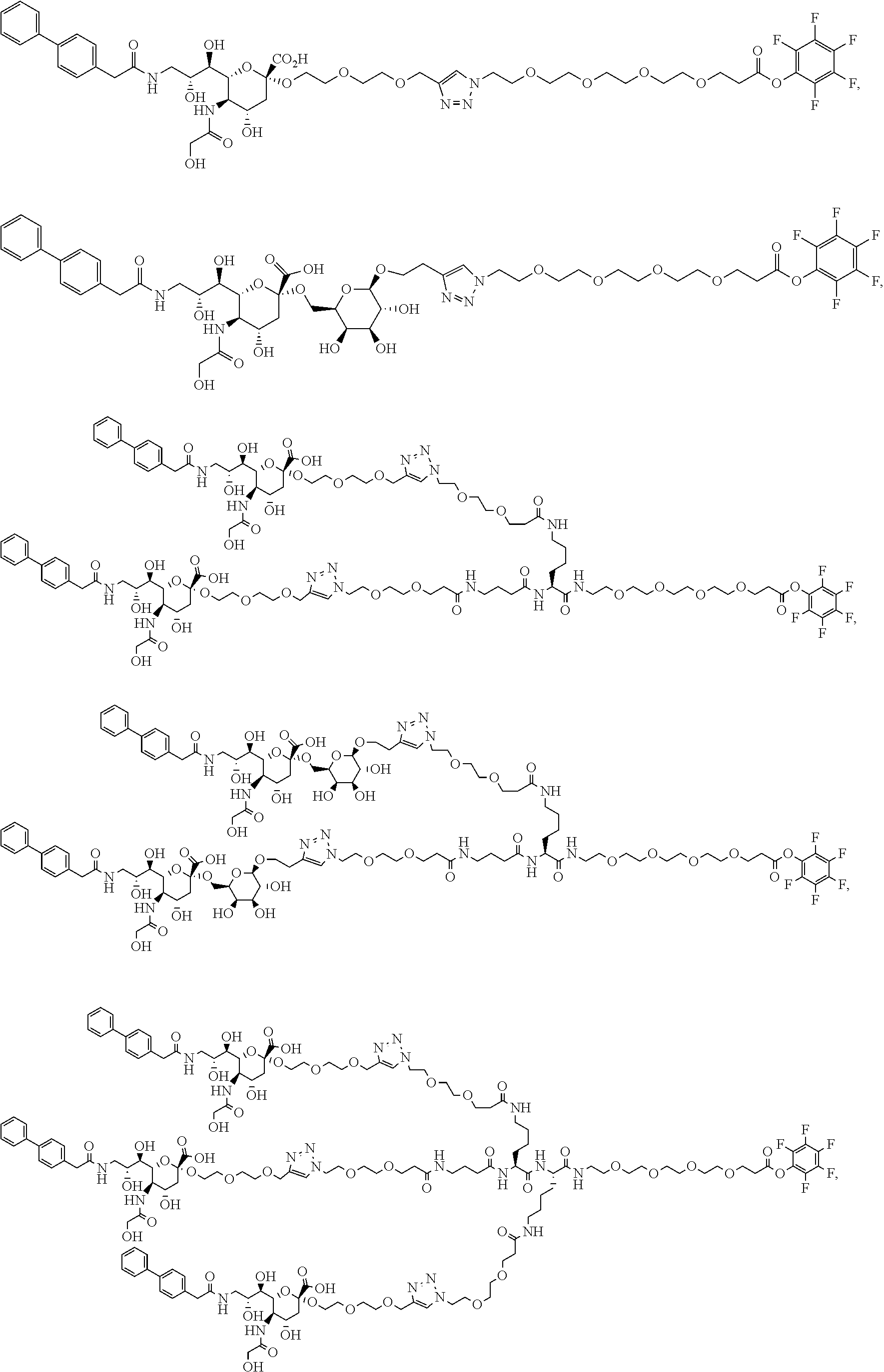

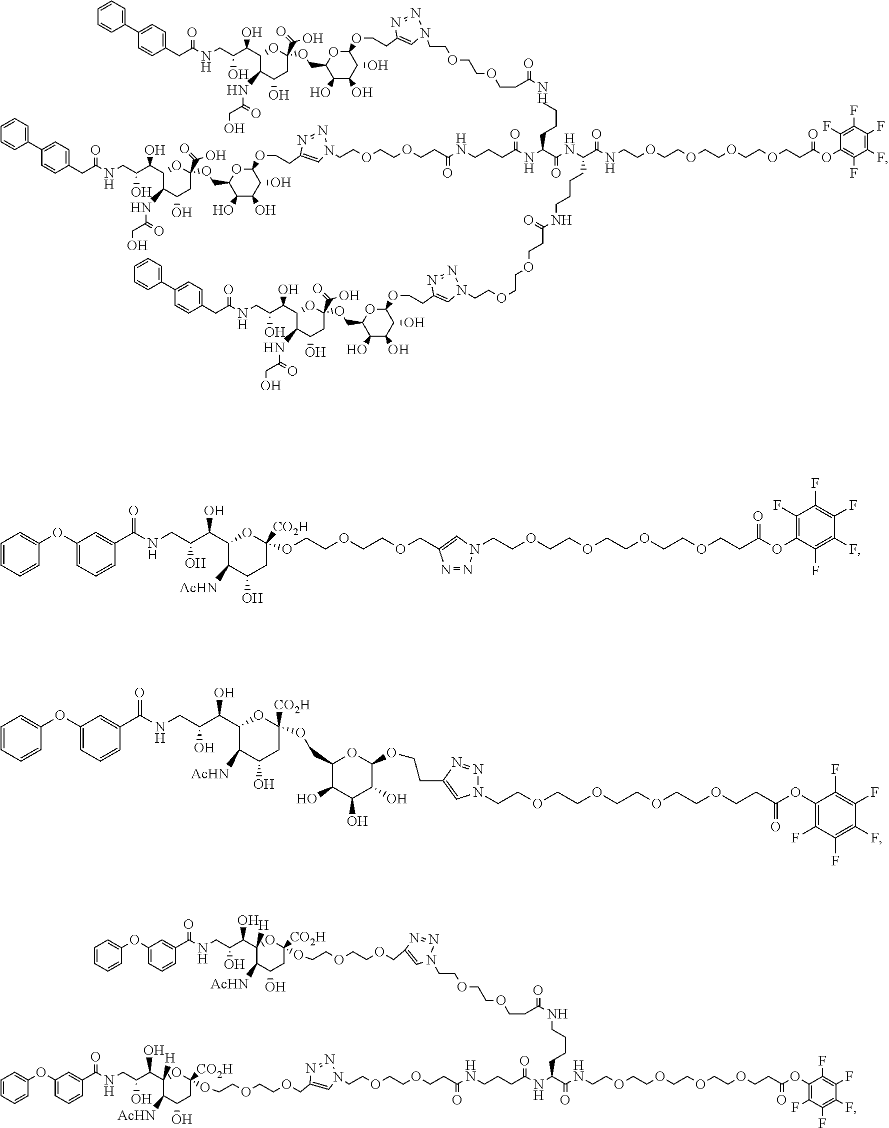

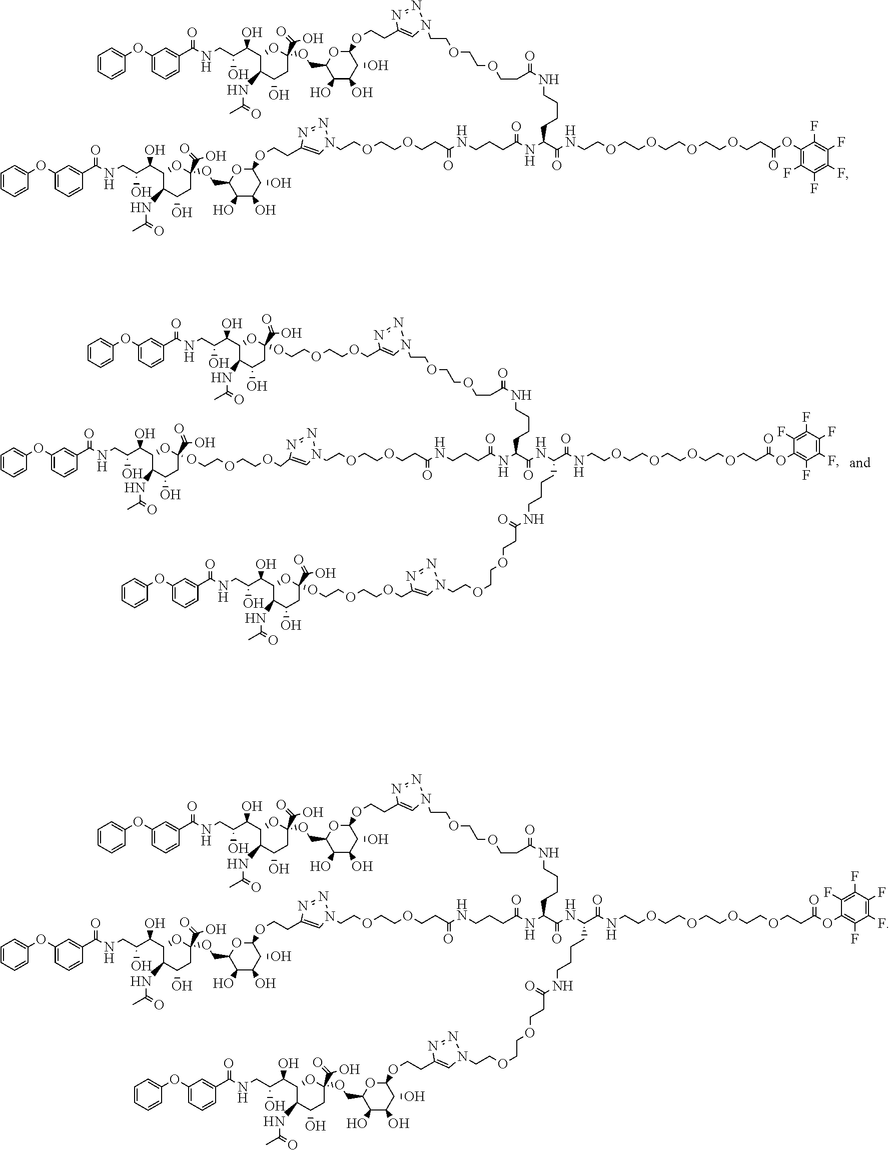

103. The engineered hypoimmunogenic biotherapeutic according to clause 101 or 102, wherein the nonnaturally occurring Siglec ligand does not comprise a saccharide between the linker and the sialic acid.

104. An engineered hypoimmunogenic biotherapeutic of formula (I):

[Xₙ-L]ₘ-Y (1)
wherein X is a sialic acid group, L is an optional linker, Y is the biotherapeutic, and n is an integer of 1 or more.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1.-104. (canceled)

105. An engineered hypoimmunogenic biotherapeutic, comprising a biotherapeutic which has been engineered to comprise an elevated amount of Sialic acid-binding immunoglobulin-type lectin (Siglec) ligand relative to a corresponding unengineered biotherapeutic while retaining therapeutic activity.

106. The engineered hypoimmunogenic biotherapeutic of claim 105, wherein the engineered hypoimmunogenic biotherapeutic is of formula (I):
[Xₙ-L]ₘ-Y (I)
wherein X is a sialic acid group, L is a linker, Y is a biotherapeutic, n is an integer of 1 or more, and m is an integer of 2 or more.

107. The engineered hypoimmunogenic biotherapeutic of claim 106, wherein in is an integer of 3 or more.

108. The engineered hypoimmunogenic biotherapeutic of claim 105, wherein the Siglec ligand comprises a linker that does not include a saccharide.

109. The engineered hypoimmunogenic biotherapeutic of claim 105, wherein the Siglec ligand comprises a sialic acid and a linker and wherein the Siglec ligand does not comprise a saccharide between the linker and the sialic acid.

110. The engineered hypoimmunogenic biotherapeutic of claim 106, wherein L comprises a polyethylene glycol group.

111. The engineered hypoimmunogenic biotherapeutic of claim 106, wherein L is a branched linker comprising one or more branching groups.

112. The engineered hypoimmunogenic biotherapeutic of claim 105, comprising one or more Siglec ligands covalently bound to the biotherapeutic, wherein the one or more Siglec ligands comprise:

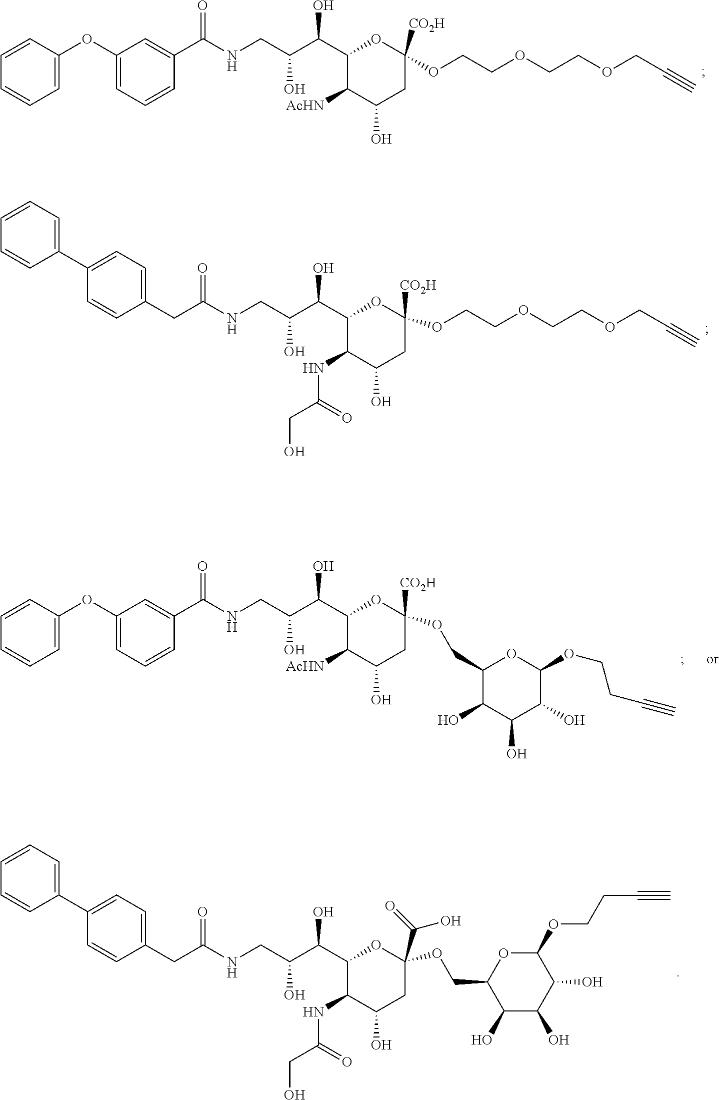 [SMILES: C#CCOCCOCCO[C@]1(C(=O)O)C[C@H](O)[C@@H](NC(C)=O)[C@H]([C@H](O)[C@H](O)CNC(=O)c2cccc(Oc3ccccc3)c2)O1 ; C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1 ; C#CCCO[C@@H]1O[C@H](CO[C@]2(C(=O)O)C[C@H](O)[C@@H](NC(C)=O)[C@H]([C@H](O)[C@H](O)CNC(=O)c3cccc(Oc4ccccc4)c3)O2)[C@H](O)[C@H](O)[C@H]1O ; C#CCCO[C@@H]1O[C@H](CO[C@]2(C(=O)O)C[C@H](O)[C@@H](NC(=O)CO)[C@H]([C@H](O)[C@H](O)CNC(=O)Cc3ccc(-c4ccccc4)cc3)O2)[C@H](O)[C@H](O)[C@H]1O]

113. The engineered hypoimmunogenic biotherapeutic of claim 112, wherein the Siglec ligand comprises a linker comprising: 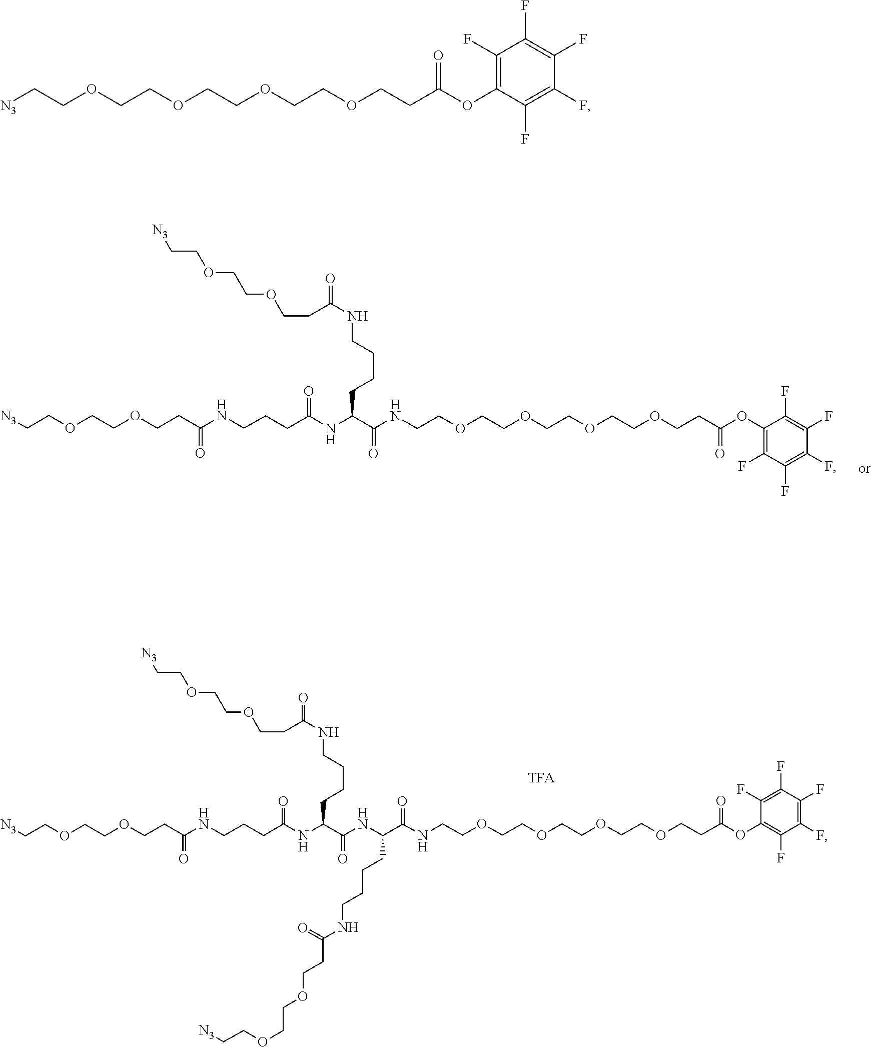 [SMILES: [N-]=[N+]=NCCOCCOCCOCCOCCC(=O)Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F]
wherein the linker attaches the sialic acid to the biotherapeutic.

114. The engineered hypoimmunogenic biotherapeutic of claim 105, wherein the Siglec ligand comprises:

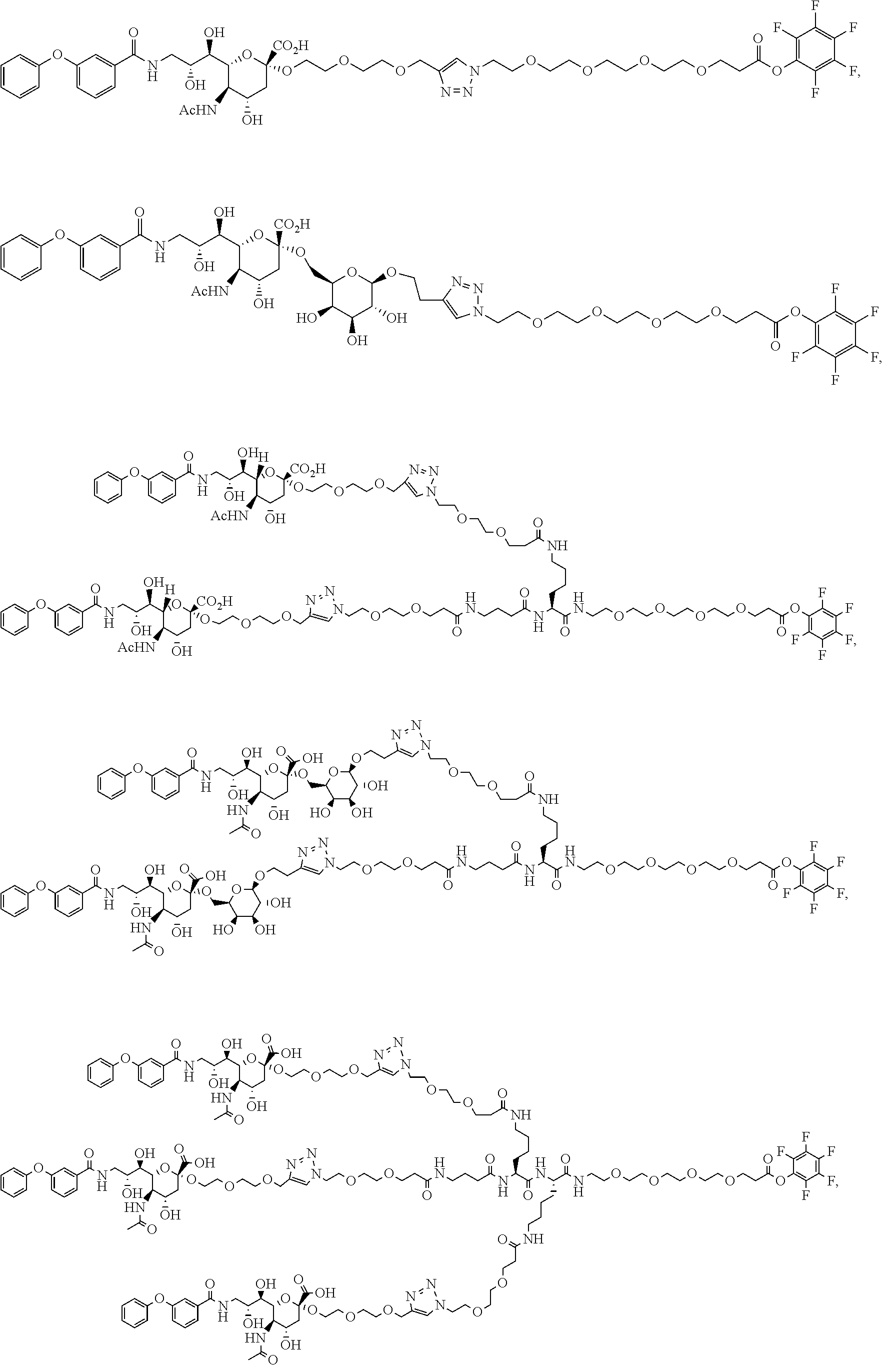

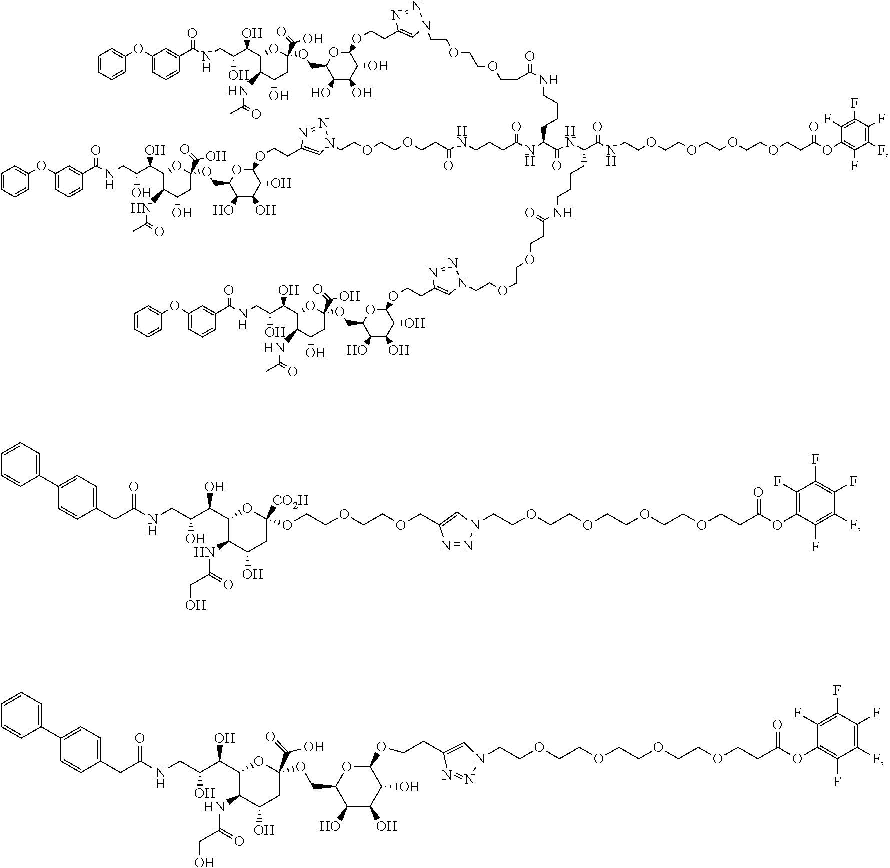

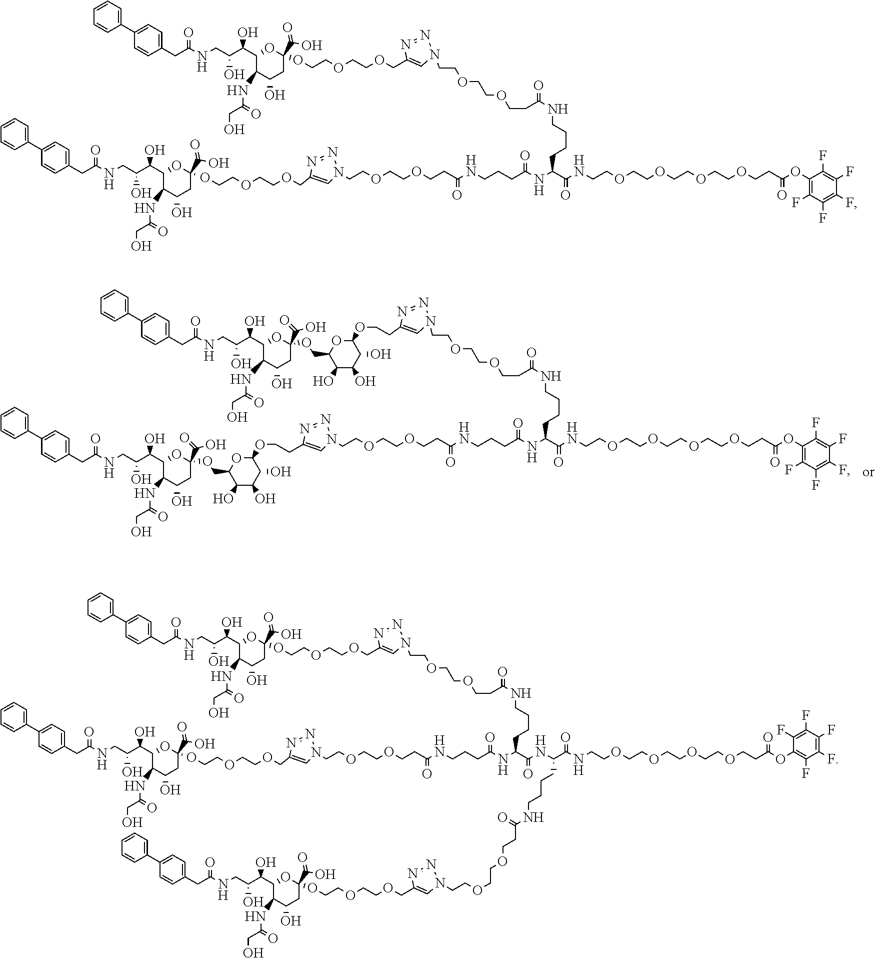

115. The engineered hypoimmunogenic, biotherapeutic f claim 105, wherein the biotherapeutic is a protein.

116. The engineered hypoimmunogenic biotherapeutic of claim 115, wherein the protein is an antibody, an enzyme, a chimeric protein, or a viral particle.

117. The engineered hypoimmunogenic biotherapeutic of claim 116, wherein the protein is an antibody and wherein the antibody adalimumab, infliximab, cetuximab, natalizumab, moxetumomab pasudotox, atezolizumab, nivolumab, abciximab, Brentuximab, Certolizumab pegol, elotuzumab, benralizumab, vedolizumab, galcanezumab, rituximab, alemtuzumab, dupilumab, golimumab, obinutuzumab, tildrakizumab, erenumab, mepolizumab, tamucirumab, ranibizumab, ustekinumab, reslizumab, ipilimumab, alirocumab, belimumab, panitumumab, avelumab, necitumumab, mogamulizumab, olaratumab, brodalumab, eculizumab, pertuzumab, pembrolizumab, or tocilizumab.

118. The engineered hypoimmunogenic biotherapeutic of claim 116, wherein the protein is an enzyme and wherein the enzyme is IdeS, asparaginase Erwinia chrysanthemi, phenylalanine ammonia-lyase, alpha-galactosidase A, acid α-glucosidase (GAA), glucocerebrosidase (GCase), aspartylglucosaminidase (AGA), alpha-L-iduronidase, iduronate sulfatase, sulfaminase, α-N-acetylglucosaminidase (NAGLU), heparin acetyle CoA: α-glucosaminide N-acetyltransferase (HGSNAT), N-acetylglucosamine 6-sulfatase (GNS), N-glucosamine 3-O-sulfatase (arylsulfatase G or ARSG), N-acetylgalactosamine 6-sulfatase, beta-galactosidase, N-acetylgalactosamine 4-sulfatase, beta-glucuronidase, Factor VIII, Factor IX, palmitoyl protein thioesterase (PPT1), Tripeptidyl peptidase (TPP1), Pseudomonas elastase (PaE), Pseudomonas alkaline protease (PaAP), or Streptococcal pyrogenic exotoxin B (SpeB).

119. A pharmaceutical composition, comprising:
the engineered hypoimmunogenic biotherapeutic of claim 105; and
a pharmaceutical excipient.

120. A method of treating an individual suffering from a disorder or disease treatable by administration of a biotherapeutic agent, the method comprising administering to the individual the pharmaceutical composition according to claim 119 in an amount effective to treat the disorder or disease, wherein the pharmaceutical composition elicits a reduced anti-drug antibody titer relative to the unengineered biotherapeutic.

121. The method of claim 120, wherein the disease is a chronic immune disease selected from the group consisting of rheumatoid arthritis, psoriatic arthritis, ankylosing spondylitis, Crohn's disease, ulcerative colitis, psoriasis, hidradenitis suppurativa, uveitis, and juvenile idiopathic arthritis, wherein the administering comprises administering to the individual an engineered hypoimmunogenic TNFα-specific antibody selected from adalimumab and infliximab in an amount effective to treat the chronic immune disease.

122. The method of claim 120, wherein the disease is leukemia, wherein the administering comprises administering to the individual an engineered hypoimmunogenic asparaginase from Erwinia chrysanthemi in an amount effective to treat the cancer.

123. The method of claim 120, wherein the disease is multiple sclerosis, wherein the administering comprises administering to the individual an engineered hypoimmunogenic natalizumab, an engineered hypoimmunogenic IFNβ-1b, or an engineered hypoimmunogenic IFNβ-1a in an amount effective to treat the multiple sclerosis.

124. The method of claim 120, wherein the disorder is an antibody response to a transplanted tissue, wherein the administering comprises administering to the individual an engineered hypoimmunogenic IdeS in an amount effective to suppress the antibody response to the transplanted tissue.

125. The method of claim 120, wherein the disorder is Type 2 Diabetes, wherein the administering comprises administering to the individual an engineered hypoimmunogenic exenatide or engineered hypoimmunogenic albiglutide in an amount effective to treat the disorder.

126. The method of claim 120, wherein the disorder is an enzyme deficiency, wherein the administering comprises administering to the individual an engineered hypoimmunogenic enzyme in an amount effective to treat the deficiency.

127. The method of claim 126, wherein the enzyme deficiency is a deficiency for an enzyme selected from the group consisting of phenylalanine ammonia-lyase (PKU), alpha-galactosidase A (for Fabry), acid α-glucosidase (GAA, for Pompe), glucocerebrosidase (GCase, for Gaucher), aspartylglucosaminidase (AGA, for Aspartylglucosaminuria), alpha-L-iduronidase (for MPS I), iduronate sulfatase (for MPS II), sulfaminase (MPS IIIa), α-N-acetylglucosaminidase (NAGLU, for MPS IIIB), heparin acetyle CoA: α-glucosaminide N-acetyltransferase (HGSNAT, for MPS IIIC), N-acetylglucosamine 6-sulfatase (GNS, for MPS IIID), N-glucosamine 3-O-sulfatase (arylsulfatase G or ARSG, MPS IIIE), N-acetylgalactosamine 6-sulfatase (for MPS IVA), beta-galactosidase (for MPS IVB), N-acetylgalactosamine 4-sulfatase (for MPS VI), beta-glucuronidase (for MPS VI), Factor VIII (for hemophilia A), Factor IX (for hemophilia B), palmitoyl protein thioesterase (PPT1, for CLN1), Tripeptidyl peptidase (TPP1, for CLN2), and cystathionine beta synthase (CBS) deficiency.

128. The method of claim 120, wherein the disorder is a monogenic disease, wherein the administering comprises administering to the individual an engineered hypoimmunogenic viral particle comprising a transgene encoding a therapeutic product in an amount effective to treat the disease.
